SEQUENCE LISTING (3 sequences)

SEQ ID NO: 1 - protein, 658 aa, Rattus norvegicus
MMPRLLFRAWPRCPSLVLGAPSRPLSAVSGPDDYLQHSIVPTMHYQDSLPRLPIPKLEDTMKRYLNAQKPLLDDSQFRRTEALCKNFETGVGKELHAHLLAQDKQNKHTSYISGPWFDMYLTARDSIVLNFNPFMAFNPDPKSEYNDQLTRATNLTVSAVRFLKTLQAGLLEPEVFHLNPSKSDTDAFKRLIRFVPPSLSWYGAYLVNAYPLDMSQYFRLFNSTRIPRPNRDELFTDTKARHLLVLRKGHFYVFDVLDQDGNIVNPLEIQAHLKYILSDSSPVPEFPVAYLTSENRDVWAELRQKLIFDGNEETLKKVDSAVFCLCLDDFPMKDLIHLSHTMLHGDGTNRWFDKSFNLIVAEDGTAAVHFEHSWGDGVAVLRFFNEVFRDSTQTPAITPQSQPAATNSSASVETLSFNLSGALKAGITAAKEKFDTTVKTLSIDSIQFQRGGKEFLKKKQLSPDAVAQLAFQMAFLRQYGQTVATYESCSTAAFKHGRTETIRPASIFTKRCSEAFVRDPSKHSVGELQHMMAECSKYHGQLTKEAAMGQGFDRHLYALRYLATARGLNLPELYLDPAYQQMNHNILSTSTLNSPAVSLGGFAPVVPDGFGIAYAVHDDWIGCNVSSYSGRNAREFLHCVQKCLEDIFDALEGKAIKT

SEQ ID NO: 2 - protein, 632 aa, Rattus norvegicus
AVSGPDDYLQHSIVPTMHYQDSLPRLPIPKLEDTMKRYLNAQKPLLDDSQFRRTEALCKNFETGVGKELHAHLLAQDKQNKHTSYISGPWFDMYLTARDSIVLNFNPFMAFNPDPKSEYNDQLTRATNLTVSAVRFLKTLQAGLLEPEVFHLNPSKSDTDAFKRLIRFVPPSLSWYGAYLVNAYPLDMSQYFRLFNSTRIPRPNRDELFTDTKARHLLVLRKGHFYVFDVLDQDGNIVNPLEIQAHLKYILSDSSPVPEFPVAYLTSENRDVWAELRQKLIFDGNEETLKKVDSAVFCLCLDDFPMKDLIHLSHTMLHGDGTNRWFDKSFNLIVAEDGTAAVHFEHSWGDGVAVLRFFNEVFRDSTQTPAITPQSQPAATNSSASVETLSFNLSGALKAGITAAKEKFDTTVKTLSIDSIQFQRGGKEFLKKKQLSPDAVAQLAFQMAFLRQYGQTVATYESCSTAAFKHGRTETIRPASIFTKRCSEAFVRDPSKHSVGELQHMMAECSKYHGQLTKEAAMGQGFDRHLYALRYLATARGLNLPELYLDPAYQQMNHNILSTSTLNSPAVSLGGFAPVVPDGFGIAYAVHDDWIGCNVSSYSGRNAREFLHCVQKCLEDIFDALEGKAIKT

SEQ ID NO: 3 - protein, 773 aa, Homo sapiens
MAEAHQAVAFQFTVTPDGIDLRLSHEALRQIYLSGLHSWKKKFIRFKNGIITGVYPASPSSWLIVVVGVMTTMYAKIDPSLGIIAKINRTLETANCMSSQTKNVVSGVLFGTGLWVALIVTMRYSLKVLLSYHGWMFTEHGKMSRATKIWMGMVKIFSGRKPMLYSFQTSLPRLPVPAVKDTVNRYLQSVRPLMKEEDFKRMTALAQDFAVGLGPRLQWYLKLKSWWATNYVSDWWEEYIYLRGRGPLMVNSNYYAMDLLYILPTHIQAARAGNAIHAILLYRRKLDREEIKPIRLLGSTIPLCSAQWERMFNTSRIPGEETDTIQHMRDSKHIVVYHRGRYFKVWLYHDGRLLKPREMEQQMQRILDNTSEPQPGEARLAALTAGDRVPWARCRQAYFGRGKNKQSLDAVEKAAFFVTLDETEEGYRSEDPDTSMDSYAKSLLHGRCYDRWFDKSFTFVVFKNGKMGLNAEHSWADAPIVAHLWEYVMSIDSLQLGYAEDGHCKGDINPNIPYPTRLQWDIPGECQEVIETSLNTANLLANDVDFHSFPFVAFGKGIIKKCRTSPDAFVQLALQLAHYKDMGKFCLTYEASMTRLFREGRTETVRSCTTESCDFVRAMVDPAQTVEQRLKLFKLASEKHQHMYRLAMTGSGIDRHLFCLYVVSKYLAVESPFLKEVLSEPWRLSTSQTPQQQVELFDLENNPEYVSSGGGFGPVADDGYGVSYILVGENLINFHISSKFSCPETDSHRFGRHLKEAMTDIITLFGLSSNSKK

(19) United States
(12) Patent Application Publication
(10) Pub. No.: US 2007/0048853 A1
(43) Pub. Date: Mar. 1, 2007

(54) Carnitine palmitoyltransferase II crystals

(76) Inventors: Joerg Benz, Rheinfelden (DE); Bernard Gsell, Lutter (FR); Michael Hennig, Weil am Rhein (DE); Arne Rufer, Heidelberg (DE); Martine Stihle, Michelbach le bas (FR); Ralf Thoma, Grenzach-Wyhlen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/507,280

(22) Filed: Aug. 21, 2006

(30) Foreign Application Priority Data
Aug. 29, 2005 (EP) 05107883.0

(51) Int. Cl. G06F 19/00; C12N 9/10; A61K 31/202; A61K 38/55

(52) U.S. Cl. 435/193; 514/2; 702/19; 514/560

(57) ABSTRACT

The present invention provides crystals of carnitine palmitoyltransferase (CPT) and co-crystals of CPT with an inhibitor compounds that allow screening for novel inhibitors of CPT-2 by determining the structure of co-crystals based on the coordinates of the crystals disclosed herein.

### PRIORITY TO RELATED APPLICATIONS

This application claims the benefit of European Application No. 05107883.0, filed Aug. 29, 2005, which is hereby incorporated by reference in its entirety.

### FIELD OF THE INVENTION

The present invention relates to the structure of full-length rat CPT-2 alone or in complex with the reversible CPT inhibitor ST1326, a substrate analog mimicking palmitoyl carnitine. The structure of CPT-2 in complex with ST1326 provides insight into the role of residues conserved in CPT-1/2 and short chain acyltransferases in protein-ligand interaction.

### BACKGROUND OF THE INVENTION

The carnitine palmitoyltransferase (CPT) system facilitates the import of long-chain fatty acids into mitochondria of higher eukaryotes. CPT-1 is integrated into the outer mitochondrial membrane and catalyzes the transfer of long chain acyl moieties from acyl-CoA to carnitine. The reverse reaction is carried out by CPT-2, a protein located on the matrix side of the inner mitochondrial membrane. Modulation of the catalytic activity of tissue-specific CPT-1 isoforms is in the focus of developing novel drugs against non-insulin dependent (type 2) diabetes mellitus and obesity.

Crystal structures of proteins are useful to identify and optimize compounds that bind to the active site of enzymes. So far, the crystal structure of CPT-2 had not been solved.

### SUMMARY OF THE INVENTION

Thus, the present invention relates to an isolated, and preferably purified, protein having the structure defined by the structural coordinates as shown in FIG. 4 or 5. The present invention also relates to three crystal forms of the protein CPT-2. One crystal of CPT-2 has unit cell dimensions of a=b=66 Å to 70 Å, c=302 Å to 312 Å; α=β=γ=90°, and the crystal has a P4₃2₁2 symmetry. In another embodiment, the crystal has unit cell dimensions of a=93 Å to 97 Å, b=94 Å to 98 Å, c=304 Å to 312 Å; α=β=γ=90°, and the crystal has a C222₁ symmetry. The present invention also relates to a co-crystal of protein CPT-2 and a ligand bound to the active site of CPT-2, wherein the co-crystal has unit cell dimensions of a=83 Å to 87 Å, b=94 Å to 98 Å, c from 121 Å to 127 Å; α=β=γ=90°, and the crystal has a P2₁2₁2₁ symmetry.

The present invention also relates to a process for co-crystallizing CPT-2 with a ligand that binds to the active site, the process comprising providing a buffered, aqueous solution of 3.75 to 50 mg/ml of CPT-2, adding a molar excess of the ligand to the aqueous solution of CPT-2, and growing crystals by vapor diffusion or microbatch using a buffered reservoir solution of 0% to 30% (w/v) PEG, wherein the PEG has an average molecular weight of 200 Da to 20000 Da. The PEG may be added as monomethyl ester. PEG may be used of an average molecular weight of 500 Da to 5,000 Da. The buffered reservoir solution further comprises 0 M to 2 M tri-ammonium citrate pH 7, 0 M to 1 M L-proline, 0 M to 1 M trimethylamine-N-oxide, 0 M to 1 M ammonium sulfate, 0 M to 1 M lithium sulfate, 0 M to 1 M ammonium acetate, 0 M to 1 M sodium or magnesium formate and 0 M to 1 M D/L-malic acid pH 7. Said microbatch may be modified. The present invention further relates to crystals produced by the process hereinbefore described.

The present invention also provides a method of identifying compounds that can bind to protein CPT-2, comprising the steps of: applying a 3-dimensional molecular modeling algorithm to the atomic coordinates of protein CPT-2 shown in FIG. 4 or 5 to determine the spatial coordinates of the binding pocket of protein CPT-2; and electronically screening the stored spatial coordinates of a set of candidate compounds against the spatial coordinates of the protein CPT-2 binding pocket to identify compound that can bind to protein P.

In an embodiment of the crystals, co-crystals, method and process hereinbefore described, said CPT-2 protein is rat CPT-2 protein. Furthermore, said rat CPT-2 protein may be the protein of Seq ID No. 2.

### DETAILED DESCRIPTION OF THE INVENTION

Definitions

As used herein, a “buffered, aqueous solution” refers to a solution based on water which comprises a substance which minimizes changes in the pH of the solution when an acid or base is added to the solution.

As used herein, a “a molar excess” refers to a larger number of ligand molecules than the number of protein molecules.

As used herein, a “vapor diffusion” refers to a protein crystallization method well known in the art wherein the crystallization solution is allowed to equilibrate with the precipitant reservoir solution through the gas phase only.

As used herein, “apo crystal” refers to crystals of CPT-2 formed without addition of site-specific ligands.

Detailed Description

Thus, the present invention relates to an isolated, and preferably purified, protein having the structure defined by the structural coordinates as shown in FIG. 4 or 5. The present invention also relates to three crystal forms of the protein CPT-2. One crystal of CPT-2 has unit cell dimensions of a=b=66 521 to 70 Å, c=302 Å to 312 Å; α=β=γ=90°, and the crystal has a P4₃2₁2 symmetry. In another embodiment, the crystal has unit cell dimensions of a=93 Å to 97 Å, b=94 Å to 98 Å, c=304 Å to 312 Å; α=β=γ=90°, and the crystal has a C222₁ symmetry. The present invention also relates to a co-crystal of protein CPT-2 and a ligand bound to the active site of CPT-2, wherein the co-crystal has unit cell dimensions of a=83 Å to 87 Å, b=94 Å to 98 Å, c from 121 Å to 127 Å; α=β=γ=90°, and the crystal has a P2₁2₁2₁ symmetry.

The present invention also relates to a process for co-crystallizing CPT-2 with a ligand that binds to the active site, the process comprising providing a buffered, aqueous solution of 3.75 to 50 mg/ml of CPT-2, adding a molar excess of the ligand to the aqueous solution of CPT-2, and growing crystals by vapor diffusion or microbatch using a buffered reservoir solution of 0% to 30% (w/v) PEG, wherein the PEG has an average molecular weight of 200 Da to 20000 Da. The PEG may be added as monomethyl ester. PEG may be used of an average molecular weight of 500 Da to 5,000 Da. The buffered reservoir solution further comprises 0 M to 2 M tri-ammonium citrate pH 7, 0 M to 1 M L-proline, 0 M to 1M trimethylamine-N-oxide, 0 M to 1 M ammonium sulfate, 0 M to 1 M lithium sulfate, 0 M to 1 M ammonium acetate, 0 M to 1 M sodium or magnesium formate and 0 M to 1 M D/L-malic acid pH 7. Said microbatch may be modified. The present invention further relates to crystals produced by the process hereinbefore described.

The present invention also provides a method of identifying compounds that can bind to protein CPT-2, comprising the steps of: applying a 3-dimensional molecular modeling algorithm to the atomic coordinates of protein CPT-2 shown in FIG. 4 or 5 to determine the spatial coordinates of the binding pocket of protein CPT-2; and electronically screening the stored spatial coordinates of a set of candidate compounds against the spatial coordinates of the protein CPT-2 binding pocket to identify compound that can bind to protein P.

In an embodiment of the crystals, co-crystals, method and process hereinbefore described, said CPT-2 protein is rat CPT-2 protein. Furthermore, said rat CPT-2 protein may be the protein of Seq ID No. 2.

The structure of CPT-2 was determined in three different crystal forms at a resolution of 2.0 Å and 1.6 Å for the apo enzyme and 2.5 Å resolution for the complex with ST1326 (see Examples for details and Table 1).

The overall fold of CPT-2 can be subdivided in an amino-terminal (residues 111-440) and a carboxy-terminal (residues 441-658, plus 32-110) domain. These domains consist of six central anti-parallel β-strands, two of which (β1, β16) mediate the major domain contact, and surrounding α-helices (FIGS. 1/2). The CPT-2 secondary structure contains 27 α-helices and 18β-strands. The loop connecting helices 22 and 23 adopts a helical conformation in the apo structure and is therefore designated helix 22B. The corresponding region of the ST1326 complex structure is located close to a crystal contact, which may interfere with secondary structure formation. A 30 amino acid insert uniquely found in CPT-2 when compared to other carnitine acyltransferases (29 amino acids in the case of L-CPT-1, FIG. 2) protrudes from the amino-terminal domain.

The electron density for the entire catalytic core of rat CPT-2 is well defined. The first 26 amino-terminal amino acids of the construct used for crystallization comprising the His-tag and five residues of the actual CPT-2 sequence are disordered in the complex and both the apo structures. The last residues with interpretable electron density at the carboxy-termini are Lys 654 (ST1326 complex) and Ile 656 (apo, P4₃2₁2), while the full carboxy-terminus is visible in chain A of the orthorhombic (C222₁) apo structure.

Apo CPT-2 and the ST1326 complex crystallized in different crystal forms but the structures have similar conformations as indicated by a r.m.s. distance of 0.38 A between equivalent Cα atoms. In all three structures the active site residue Arg 498 is in the generously allowed region of the Ramachandran plot and Leu 129 as well as Asn 230 do not comply with favoured geometry of the Ramachandran plot, but unambiguous electron density is visible for these residues. Leu 129 is the second residue in a type II reverse turn (equivalent to Ile 116 in mouse CrAT, PDB code 1t7n), whereas Asn 230 is located in a β-turn whose geometry may be distorted due to interaction with neighboring Asp 297 and Arg 124. Mutation of Arg 124 is associated with inherited CPT-2 deficiency (see below).

Thus, the present invention relates to an isolated, and preferably purified, protein having the structure defined by the structural coordinates as shown in FIG. 4 or 5. The present invention also relates to three crystal forms of the protein CPT-2. One crystal of CPT-2 has unit cell dimensions of a=b=66 Å to 70 Å, c=302 Å to 312 Å; α=β=γ=90°, and the crystal has a P4₃2₁2 symmetry. In another embodiment, the crystal has unit cell dimensions of a=93 Å to 97 Å, b=94 Å to 98 Å, c=304 Å to 312 Å; α=β=γ=90°, and the crystal has a C222₁ symmetry. The present invention also relates to a co-crystal of protein CPT-2 and a ligand bound to the active site of CPT-2, wherein the co-crystal has unit cell dimensions of a=83 Å to 87 Å, b=94 Å to 98 Å, c from 121 Å to 127 Å; α=β=γ=90°, and the crystal has a P2₁2₁2₁ symmetry.

TABLE 1
Data collection and refinement statistics

| | apo, | | |
|---|---|---|---|
| | high resolution | apo2 | CPT-II/ST1316 |
| Data collection | | | |
| Space group | C222₁ | P4₃2₁2 | P2₁2₁2₁ |
| Cell dimensions | | | |
| a, b, c (Å) | 95.2, 97.3, | 67.6, 67.6, 307.3 | 85.8, 96.2, 124.3 |
| | 310.4 | | |
| α, β, γ (°) | 90.0, 90.0, | 90.0, 90.0, 90.0 | 90.0, 90.0, 90.0 |
| | 90.0 | | |
| Resolution (Å)* | 23.0-1.6 | 23.0-2.0 | 50.0-2.24 |
| | (1.69-1.60) | (2.12-2.00) | (2.38-2.24) |
| R_{merge} | 6.2 (21.2) | 3.1 (7.4) | 16.7 (41.1) |
| I/σI | 14.04 (7.24) | 28.04 (14.80) | 9.57 (3.20) |
| Completeness (%) | 99.7 (99.8) | 99.9 (100) | 92.9 (81.8) |
| Redundancy | 14.7 (17.3) | 21.8 (18.5) | 6.9 (6.2) |
| Refinement | | | |
| Resolution (Å) | 23-0-1.6 | 23.0-2.0 | 15.0-2.5 |
| No. reflections | 179022 | 46957 | 32831 |
| R_{work}/R_{free} | 16.5/19.4 | 17.9/23.5 | 24.1/29.6 |
| No. atoms (all) | 11449 | 5502 | 5143 |
| Protein | 10085 | 4977 | 4963 |
| Ligand/ion | n/a* | n/a | 28 |
| Water | 1364 | 525 | 152 |
| B-factors | 20.1 | 21.9 | 41.0 |
| (overall) | | | |
| Protein | 18.7 | 21.0 | 41.4 |
| Ligand/ion | n/a* | n/a | 27.6 |
| Water | 30.1 | 30.7 | 31.6 |
| R.m.s deviations | | | |
| Bond lengths (Å) | 0.014 | 0.01 | 0.013 |
| Bond angles (°) | 1.225 | 1.161 | 1.502 |

*The βOG detergent molecule (mean B = 54.9 Å²), the two C16 alkyl-moieties (mean B = 23.3 Å²) and the two residual, unspecifically bound CoA molecules (mean B = 37.7 Å²) were not included in the statistic.

The CPT inhibitor ST1326 binds to the active site of CPT-2 which is located in a tunnel penetrating CPT-2 at the domain interface (FIG. 3). ST1326 is a non-cleavable analog of palmitoyl-carnitine, the physiological substrate of CPT-2. The acyl- and carnitine tunnel of the tripartite (Nic a' Bhaird, N., et al., Comparison of the active sites of the purified carnitine acyltransferases from peroxisomes and mitochondria by using a reaction-intermediate analogue. Biochem J., 294, 645-651 (1993)) active site of CPT-2 are occupied by ST1326, whereas the CoA-tunnel can be assigned by homology modeling based on the complex structure of CrAT with CoA (FIG. 3). The hydrophilic aminocarnitine head group of ST1326 is tightly bound in a hydrogen-bond network. The catalytic base His 372 forms a hydrogen bond with the amino-nitrogen (N11) of ST1326, which substitutes the ester oxygen of the native ligand palmitoyl-carnitine. Ser 590 of the Ser-Thr-Ser motif conserved among carnitine acyltransferases makes a hydrogen bond to the carbamoyl-oxygen (O13) of the ligand. Tyr 486, Ser 488 and Thr 499 of the carboxy-terminal domain are directly hydrogen-bonded to the carboxyl oxygens (O9 and O10) of ST1326. Hydrogen bonds to the guanidinium group of Arg 554 further stabilize the orientation of Tyr 486 and Thr 499. Residues Trp 116, Tyr 120 and Asp 376 of the amino-terminal domain and a concertedly fixed water molecule are also part of the hydrogen bond network binding to the carboxy group of ST1326. Arg 498 forms a strong hydrogen bridge with the side chain of Asp 376 and its guanidinium group interacts with the main chain carbonyl oxygen of Ser 373 in the catalytic loop, thereby rendering the active site residues in a position ideal for catalysis. The positively charged tertiary amine of ST1326 is stabilized by cation-pi interactions with the conserved Phe 602.

The hydrophobic tunnel that accommodates the aliphatic tetradecanoyl tail of ST1326 is lined by residues of β-strands 1 and 16, which form an anti-parallel β-sheet at the domain interface, and the two carboxy-terminal β-strands 17 and 18. A simulated annealing fofc map contoured at 2.5 σ shows clear electron density for the ligand ST1326 bound to the active site. The β-strands forming the hydrophobic tunnel are moved apart in order to make room for the extended hydrophobic tail of the substrate analog ST1326 compared to the closed arrangement in CrAT and CrOT (PDB codes 1ndb, 1t7n and 1xl8). The glycine residue Gly 600 at a position where bulkier residues are found in CrAT (Met 564) and CrOT (Gln 552) allows binding of LCFA carnitine-derivatives in CPT-2, thereby determining substrate specificity. In contrast to CrAT and CrOT the acyl-tunnel opens to the surface in CPT-2.

Glu 487 and Glu 500 of CPT-2, which are conserved throughout the carnitine acyltransferases, have been implicated in substrate binding and catalysis by means of mutant analysis (Zheng, G., et al., Identification by mutagenesis of a conserved glutamate (Glu487) residue important for catalytic activity in rat liver carnitine palmitoyltransferase II. J. Biol. Chem., 277, 42219-42223 (2002)). The crystal structure of CPT-2 reveals that Glu 487 indeed is located in the part of the active site tunnel that accommodates CoA. Glu 487 together with the highly conserved Asp 464 form a negatively charged patch that is probably required for guiding substrates to the active site. The side chain of Glu 500 interacts with the main chain of conserved Arg 554, which is a crucial component of the hydrogen network required for binding the carnitine moiety of acyltransferase substrates.

In the apo structures the side chains of Tyr 120 and the catalytic His 372 are disordered despite the higher quality of the data set and resolution of the structure analysis. Those residues in the active site that interact with the hydrophilic aminocarnitine head group of ST1326 as well as Ser 590 are moved away from the ligand binding, but the overall shape of the active site tunnel is preformed in the apo enzyme.

The clinically heterogeneous disease CPT-2 deficiency is caused by various mutations in the CPT-2 gene and is inherited in an autosomal recessive manner (Bonnefont, J-P. et al. Carnitine palmitoyltransferases 1 and 2: biochemical, molecular and medical aspects. Mol. Aspects Med., 25, 495-520 (2004); Bonnefont, J. P. et al. Carnitine palmitoyltransferase deficiencies. Mol. Genet. Metab., 68, 424-440 (1999)). Two different manifestations of the disorder can be distinguished based on the time of onset, i.e., the early onset (neonatal or infantile) and the more frequent adult form of CPT-2 deficiency. The early onset form is characterized by a severe symptomatology including cardiomyopathy and hypoketotic hypoglycemia and has been linked to acute liver failure in sudden infant death syndrome (Demaugre, F. et al. Infantile form of carnitine palmitoyltransferase II deficiency with hepatomuscular symptoms and sudden death. Physiopathological approach to carnitine palmitoyltransferase II deficiencies. J. Clin. Invest., 87, 859-864 (1991)). Clinical signs of adult CPT-2 deficiency are recurrent myalgia and myoglobinuria in response to fasting and exercise.

More than 30 point mutations in the coding region for CPT-2 leading to single amino acid exchanges in the enzyme have been identified in addition to deletions/insertions causing frame shifts or truncation of the protein. Whereas truncation of the CPT-2 gene inevitably leads to a severe neonatal presentation of CPT-2 deficiency by loss of CPT-2 function, a graduated correlation of genotype and severity of clinical phenotype becomes obvious for those miss-sense mutations that have been described in a homozygous state (Table 2) (Bonnefont, J. P. et al. Carnitine palmitoyltransferase deficiencies. Mol. Genet. Metab., 68, 424-440 (1999); Thuillier, L. et al. Correlation between genotype, metabolic data, and clinical presentation in carnitine palmitoyltransferase 2 (CPT2) deficiency. Hum. Mutat., 21, 493-501 (2003)). An exception to this correlation is the Arg 631 Cys mutation, which has been identified in homozygous patients both with the adult as well as with the infantile form of CPT-2 deficiency. The crystal structure of CPT-2 allows mapping and interpretation of the effects of the described mutations (summarized in Table 2). Most (60%) cases of adult CPT-2 deficiency are associated with a Ser 113 Leu mutation. Ser 113 is located at the amino-terminus of helix α5 close to the domain interface. The side chain of Ser 113 does not make any direct contacts to surrounding amino acids but mutation of this residue to a larger, hydrophobic Leu alters the interaction with the neighboring Phe 117. This changes the position and environment of the catalytically important residues Trp 116 and Arg 498, rendering the enzyme less active (Taroni, F. et al. Identification of a common mutation in the carnitine palmitoyltransferase II gene in familial recurrent myoglobinuria patients. Nat. Genet., 4, 314-320 (1993)).

Asp 213 and Glu 487 are affected by naturally occurring mutations and have been determined to be important for CPT-2 function by biochemical analyses (Zheng, G., et al., Identification by mutagenesis of a conserved glutamate (Glu487) residue important for catalytic activity in rat liver carnitine palmitoyltransferase II. J. Biol. Chem., 277, 42219-42223 (2002); Liu, H., et al., Cysteine-scanning mutagenesis of muscle carnitine palmitoyltransferase I reveals a single cysteine residue (Cys-305) is important for catalysis. J. Biol. Chem., 280, 4524-4531 (2005)). Asp 213 is located in a loop between β3 and α10 of CPT-2 and aligns with a cysteine conserved in all human CPT-1 isoforms. Mutation of this cysteine to Ala fully abolishes enzyme activity in human M-CPT-1, indicating that this position is crucial for structural integrity in all CPT isoforms. In CPT-2 the side chain of Asp 213 makes a strong interaction with the main chain nitrogen of His 496. This is important for the positioning Arg 498 and Arg 499 which are involved in substrate binding. Mutation of Glu 487 in β13 to aspartic acid leads to an almost complete loss of CPT-2 activity. This mutation could interfere with CoA binding as Glu 487 is part of the CoA-tunnel surface. From the CPT-2 crystal structure it can be predicted that an aspartic acid at position 487 would form a strong hydrogen bond with the side chain of Thr 589 of the conserved STS motif, thereby distorting the geometry of the active site. A Glu 487 Lys exchange is one of 6 mutations identified in CPT-2 deficiency that cause disruption of internal salt bridges or hydrogen-bond interactions which are fully conserved in CPT-1, CrAT and CrOT (Table 2). A guanidinium nitrogen of Arg 296 makes a strong (2.7 Å) contact with a side chain oxygen of Asp 353, which is conserved in carnitine acyltransferases. The equivalent residue in M-CPT-1, Asp 454, has been proposed to be part of a catalytic triad (Liu, H., et al., Cysteine-scanning mutagenesis of muscle carnitine palmitoyltransferase I reveals a single cysteine residue (Cys-305) is important for catalysis. J. Biol. Chem., 280, 4524-4531 (2005)), whereas the CPT-2 crystal structure implicates this residue to form a conserved salt bridge. None of the reported mutants associated with CPT-2 deficiency is located in the insert, although they are distributed uniformly in the tertiary structure.

Table 2 Residues mutated in human CPT-2 deficiency. All the affected residues are conserved between rat and human CPT-2. Residue numbering for the human and rat isoforms of CPT-II are identical. Homozygous mutations labeled A are associated with the late onset, adult form of CPT-2 deficiency, those labeled I with the early onset, infantile presentation.

| Mutation | Effect on | | Conservation | | |
|---|---|---|---|---|---|
| P50H | structure of amino-terminus | A | CPT-1 | CrAT | CrOT |
| L72F | structure of amino-terminus (M194 in | | M & B | | |
| | lCPT-I; V58, A43, i.e. small | | CPT-1 | | |
| | hydrophobic residue, in CrAT and | | | | |
| | CrOT respectively) | | | | |
| S113L | enviroment of catalytically important | A | CPT-1 | CrAT | |
| | Trp 116 and Arg 498, domain interface | | | | |
| | (most frequent mutation found in | | | | |
| | adult CPT-2 deficiency) | | | | |
| Y120C | substrate (carnitine moiety) binding | | CPT-1 | CrAT | CrOT |
| R124Q | folding of amino-terminal domain | | CPT-1 | CrAT | CrOT |
| R124X | conserved interaction with D232 | | | | |
| N146T | folding of amino-terminal domain | | | | |
| R151Q | folding of amino-terminal domain | I | CPT-1 | | CrOT |
| R161W | domain interface and interaction with | A | | | |
| | amino-terminus | | | | |
| K164X | interaction with amino-terminus | | CPT-1 | | |
| | (basic residue in CPT-1) | | R or H | | |
| E174 K | folding of amino-terminal domain | A | | CrAT | |
| | (residue interacts with R382), altered | | | | |
| | surface of CoA binding site | | | | |
| Y210D | close to insertion, i.e. involved in | | | | |
| | membrane association | | | | |
| D213G | domain interface, substrate binding | | | | CrOT |
| | (long range disruption of hydrogen | | | | |
| | network), C305 in human muscle | | | | |
| | CPT-1 ( . . . ) | | | | |
| M214T | domain interface and interaction with | | | CrAT | CrOT |
| | amino terminus | | | | |
| P227L | folding of amino-terminal domain | I | CPT-1 | CrAT | CrOT |
| K274M | folding of amino-terminal domain | | | | |
| Y290X | folding of amino-terminal domain | | | | |
| R296Q | folding of amino-terminal domain | | CPT-1 | CrAT | CrOT |
| R296X | conserved interaction with D353 | | | | |
| D328G | folding of amino-terminal domain | I | CPT-1 | CrAT | CrOT |
| | conserved interaction with R350 | | (E) | | (E279) |
| R382K | folding of amino-terminal domain | I | CPT-1 | | |
| | (residue interacts with E174) | | (H) | | |
| F383Y | folding of amino-terminal domain | I | | | |
| F448L | folding of carboxy-terminal domain, | | CPT-1 | CrAT | CrOT |
| | surface at entry of acyl-tunnel | | | | |
| E454X | substrate (CoA moiety) binding | | | CrAT | |
| | | | | (D420) | |
| Y479F | folding of carboxy-terminal domain | | | CrAT | |
| E487K | surface of CoA binding site ( . . . ) | | CPT-1 | CrAT | CrOT |
| | conserved interaction with H539 | | | | |
| I502T | substrate binding (long range | | CPT-1 | CrAT | CrOT |
| | disruption of hydrogen network) | | (V) | | (V455) |
| R503C | folding of carboxy-terminal-domain, | | CPT-1 | CrAT | CrOT |
| | surface at entry of carnitine-tunnel | | | | |
| | conserved interaction with D553 | | | | |
| G549D | folding of carboxy-terminal domain, | | CPT-1 | CrAT | CrOT |
| | substrate binding (long range | | | | |
| | disruption of hydrogen network), | | | | |
| Q550R | folding of carboxy-terminal domain, | | B | | |
| | possibly binding of CoA | | CPT-1 | | |
| | | | (Q648) | | |
| D553N | substrate binding (long range | | CPT-1 | CrAT | CrOT |
| | disruption of hydrogen network), surface | | | | |
| | at entry of carnitine-tunnel, conserved | | | | |
| | interaction with R503 | | | | |
| P604S | domain interface, substrate binding | | CPT-1 | CrAT | CrOT |
| | (alignment of F602) | | | | |
| D608H | folding of carboxy-terminal domain | | L & M | CrAT | |
| | interaction with H584 (c | | CPT-1 | | |
| Y628S | domain interface | I | | | CrAT |
| R631C | folding of carboxy-terminal domain | I/A | | | |

The apo, derivative and co-crystals of the invention can be obtained by techniques well-known in the art of protein crystallography, including batch, (modified) micro-batch, liquid bridge, dialysis, vapor diffusion and hanging drop methods (see e.g. McPherson, 1982, Preparation and Analysis of Protein Crystals, John Wiley, NY; McPherson, 1990, Eur. J. Biochem. 189:1-23; Webber, 1991, Adv. Protein Chem. 41:1-36; Crystallization of Nucleic Acids and Proteins, Edited by Arnaud Ducruix and Richard Giege, Oxford University Press; Protein Crystallization Techniques, Strategies, and Tips, Edited by Terese Bergfors, International University Line, 1999). Generally, the apo- or co-crystals of the invention are grown by placing a substantially pure CPT-2 polypeptide in an aqueous buffer containing a precipitant at a concentration just below that necessary to precipitate the protein. Water is then removed from the solution by controlled evaporation to produce crystallizing conditions, which are maintained until crystal growth ceases.

In a preferred embodiment of the invention, apo or co-crystals are grown by vapor diffusion. In this method, the polypeptide/precipitant solution is allowed to equilibrate in a closed container with a larger aqueous reservoir having a precipitant concentration optimal for producing crystals. Generally, less than about 10 μL of substantially pure polypeptide solution is mixed with an equal or similar volume of reservoir solution, giving a precipitant concentration about half that required for crystallization. This solution is suspended as a droplet underneath a coverslip, which is sealed onto the top of a reservoir. The sealed container is allowed to stand, from one day to one year, usually for about 2-6 weeks, until crystals grow.

For crystals of the invention, it has been found that hanging drops containing about 2-5 μl of rat CTP-2 [3.75-50 mg/ml rat CPT-2 in 25 mM Tris/HCl pH 8.0, 0.15 M NaCl, 2 mM TCEP, 1% (w/v) n-octy-β-D-glucopyranoside (βOG) or 0.33% (w/v) CHAPS or 1.15 mM n-dodecyl-β-D-maltoside (DBM) and 0.02% (w/v) NaN₃] grown at room temperature (21 C. +/−2° C.) and an equal or similar amount of reservoir solution (Index 21, 37, 61, 62, 69, 75, 76, 79, 88, 90, 91, 92, Hampton Research, as well as solutions derived from these by altering the pH or the concentrations of their ingredients) suspended over 0.33 to 1.0 mL reservoir buffer for about 1-7 days provided crystals suitable for high resolution X-ray structure determination. Particularly preferred conditions were: a (modified) microbatch assay with about 0.1 μl-1 μl of rat CTP-2 (13.5 mg/ml in 25 mM Tris/HCl pH 8.0, 0.15 M NaCl, 2 mM TCEP and 1% (w/v) β-D-OG) pre-incubated with a 3-fold molar excess of ST1326 as well as hanging drop assays with 1 μl to 5 μl rat CPT-2 (12 mg/ml, from conventional expression or as protein labeled with seleno-methionene, in 25 mM Tris/HCl pH 8.0, 0.15 M NaCl, 2 mM TCEP and 1% (w/v) β-D-OG) mixed with 0.5 μl to 2 μl of Index 91 (Hampton Research) precipitant solution over a 0.33 ml to 1.0 ml reservoir solution (Index 91, Hampton Research).

All crystal grown in hanging drops were submerged in the respective mother liquor supplemented with 20% (w/v) to 30% (w/v) polyethylene glycol 200 (PEG 200) and appropriate ligand-concentration in the case of complex structures prior to freezing in liquid nitrogen or mounting on a goniometer equipped with a liquid nitrogen cooling device. The crystals grown using the microbatch method were either frozen as described for the crystals grown in hanging drops or the mother liquor and oil surrounding the crystal was exchanged against 100% (v/v) paraffin oil.

Diffraction data typically extending to 2.0 Å was collected from the frozen crystals at the synchrotron beamline X10SA (PX1) or X10SA (PX2) at the SLS, Villigen, Switzerland. Under optimum conditions, data extending to 1.6 Å was recorded.

Of course, those of ordinary skill in the art will recognize that the above-described crystallization conditions can be varied. Such variations may be used alone or in combination, and include polypeptide solutions containing polypeptide concentrations between 1 mg/mL and 60 mg/mL, any commercially available buffer systems which can maintain pH from about 3.0 to about 11.0, reducing components in various concentrations (dithiothreitol, dithioeritol, β-mercapto ethanol or other art-recognized equivalents), stabilizing components like sugar or glycerols in concentrations between 0% (w/v) and 30% (w/v) or substitution of other ligands known to bind CPT-2. Precipitant and reservoir solutions containing polyethylene glycol (PEG) concentrations between about 1% (w/v) and about 30% (w/v), polyethylene glycol average molecular weights between about 200 and about 20,000 daltons, and temperature ranges between 4° C. and 30° C.

Thus, the present invention also relates to a process for co-crystallizing CPT-2 with a ligand that binds to the active site, the process comprising providing a buffered, aqueous solution of 3.75 to 50 mg/ml of CPT-2, adding a molar excess of the ligand to the aqueous solution of CPT-2, and growing crystals by vapor diffusion or microbatch using a buffered reservoir solution of 0% to 30% (w/v) PEG, wherein the PEG has an average molecular weight of 200 Da to 20000 Da. The PEG may be added as monomethyl ester. PEG may be used of an average molecular weight of 500 Da to 5,000 Da. The buffered reservoir solution further comprises 0 M to 2 M tri-ammonium citrate pH 7, 0 M to 1 M L-proline, 0 M to 1 M trimethylamine-N-oxide, 0 M to 1 M ammonium sulfate, 0 M to 1 M lithium sulfate, 0 M to 1 M ammonium acetate, 0 M to 1 M sodium or magnesium formate and 0 M to 1 M D/L-malic acid pH 7. Said microbatch may be modified. The present invention further relates to crystals produced by the process hereinbefore described.

Derivative crystals of the invention can be obtained by soaking apo or co-crystals in mother liquor containing salts of heavy metal atoms, according to procedures known to those of skill in the art of X-ray crystallography.

Co-crystals of the invention can be obtained by soaking an apo crystal or a crystal of CPT-2 with a low affinity ligand in mother liquor or any other stabilizing solution containing a ligand that binds to the active site, or can be obtained by co-crystallizing the CPT-2 polypeptide in the presence of one or more ligands that bind to the active site.

Methods for obtaining the three-dimensional structure of the crystalline CPT-2 described herein, as well as the atomic structure coordinates, are well-known in the art (see, e.g., D. E. McRee, Practical Protein Crystallography, published by Academic Press, San Diego (1993), and references cited therein).

The crystals of the invention, and particularly the atomic structure coordinates obtained therefrom, have a wide variety of uses. For example, the crystals and structure coordinates described herein are particularly useful for identifying compounds that inhibit or generally modify the activity of CPT-2 as an approach towards developing new therapeutic agents.

The structure coordinates described herein can be used as phasing models in determining the crystal structures of additional native or mutated CPT-2, as well as the structures of co-crystals of such CPT-2 with inhibitors or activators bound. The structure coordinates, as well as models of the three-dimensional structures obtained therefrom, can also be used to aid the elucidation of solution-based structures of native or mutated CPT-2, such as those obtained via NMR. Thus, the crystals and atomic structure coordinates of the invention provide a convenient means for elucidating the structures and functions of CPT-2.

Thus, the present invention also provides a method of identifying compounds that can bind to protein CPT-2, comprising the steps of: applying a 3-dimensional molecular modeling algorithm to the atomic coordinates of protein CPT-2 shown in FIG. 4 or 5 to determine the spatial coordinates of the binding pocket of protein CPT-2; and electronically screening the stored spatial coordinates of a set of candidate compounds against the spatial coordinates of the protein CPT-2 binding pocket to identify compound that can bind to protein P.

In an embodiment of the crystals, co-crystals, method and process hereinbefore described, said CPT-2 protein is rat CPT-2 protein. Furthermore, said rat CPT-2 protein may be the protein of Seq ID No. 2.

For purposes of clarity and discussion, the crystals of the invention will be described by reference to specific CPT-2 exemplary apo crystals and co-crystals. Those skilled in the art will appreciate that the principles described herein are generally applicable to crystals of any mammalian CPT-2, including, but not limited to the CPT-2 of Seq ID No. 2.

### BRIEF DESCRIPTION OF THE FIGURES

FIG. 1. Structure of rat CPT-2 with ST1326 bound to the active site. The ligand ST1326 and its surrounding fofc simulated annealing electron density map (contoured at 2.5 σ) are depicted in pink in A-C. A, ST1326 binds at the interface of the amino-terminal (orange) and carboxy-terminal (cyan) domains of rat CPT-2. B, the central β-strands (blue) are surrounded by α-helices (green), the CPT-2-specific insert (red) protrudes from the amino-terminal domain. C, same as B but rotated 90° to the back.

FIG. 2: Amino acid sequence alignment of rat CPT-2 (rCPT-2) and human CPT-1 (hCPT-1). Secondary structure elements (S.S.) are indicated. The residue numbering corresponds to the rCPT-2 precursor and its mitochondrial import sequence is italicized. The CPT-2 specific insert (amino acids 179-208) is underlined. Key residues of the acylcarnitine binding site of rCPT-2 are in bold letters and are labeled with * when fully conserved in hCPT-1. Residues that have been reported to be mutated in CPT-2 deficiency are printed red.

FIG. 3. A, stereo figure of the active site tunnel viewed perpendicular to the domain interface. Key active site residues are depicted in yellow. The co-crystallized ST1326 is shown in pink, a CoA molecule (blue) was modeled based on the CoA coordinates from the CrAT-CoA complex structure (PDB code 1t7q). B, Fischer projection of ST1326 with atom numbering.

FIG. 4: Coordinates of apol (C222₁) crystal

FIG. 5: Coordinates of apol (P43212) crystal

FIG. 6: Coordinates of CPT-2/ST1316 co-crystal

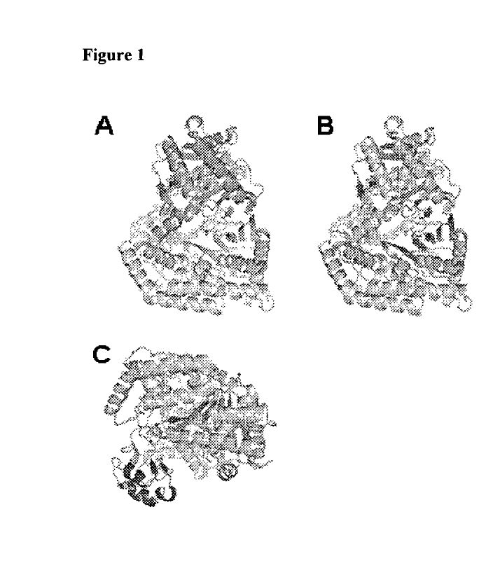

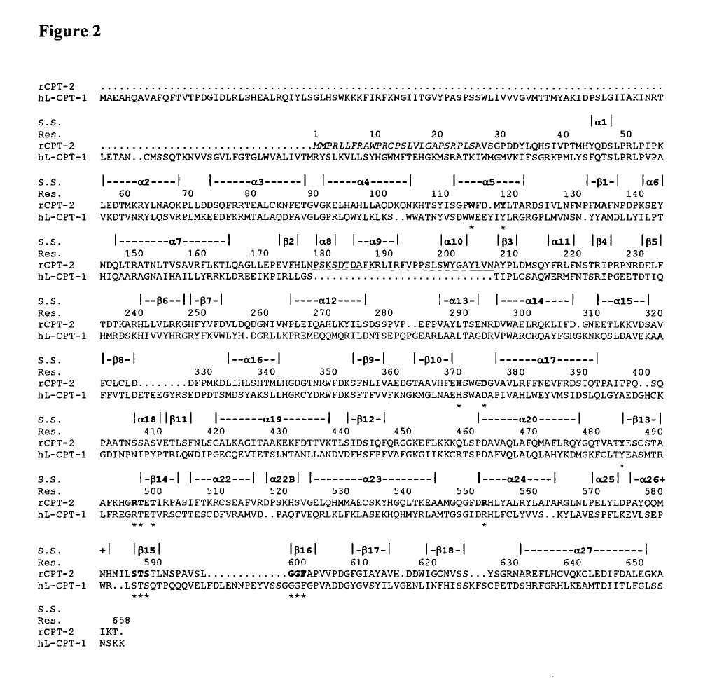

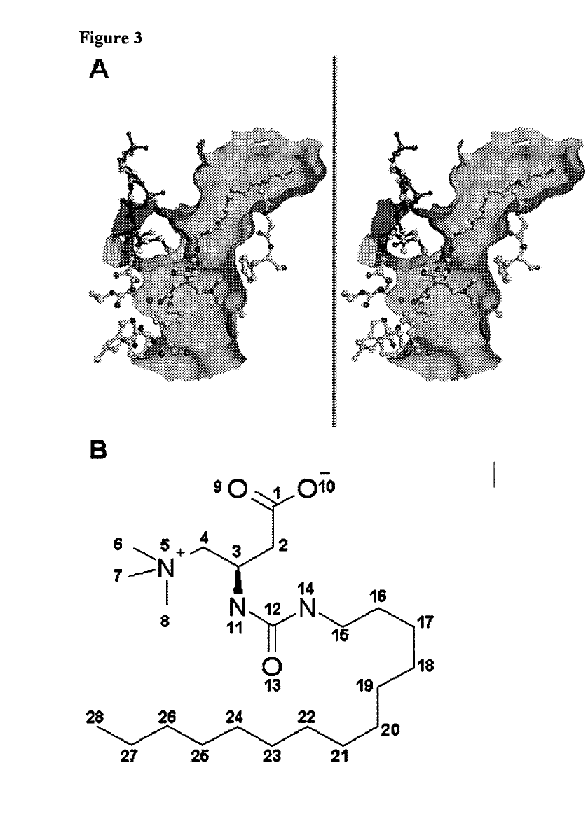

### EXAMPLES

### Example 1

### Protein Expression and Purification

The DNA coding for amino acids 27-658 of rat CPT-2 (a kind gift by Dr. V. A. Zammit, Hannah Research Institute, Ayr, Scotland) was subcloned into a Novagen pET28a vector. This construct was used to express full-length CPT-2 (exempt from the mitochondrial import sequence, amino acids 1-26 of the CPT-2 precursor) in E. coli strain BL21DE3 at 20° C. After cell disruption the lysate (in 50 mM HEPES/NaOH pH 8, 0.15 M NaCl, 5 mM TCEP, 10 mM MgCl₂, 30 mg/l DNase I, 30 Tbs./l Roche Complete protease inhibitor) was adjusted with 0.1% (v/v) Triton-X-100. Solubilization and centrifugation (30,000 g, 45 min) was followed by immobilized metal affinity chromatography on a Ni-NTA resin. The detergent was exchanged to 1% (v/v) n-octyl-β-D-glucopyranoside (βOG) on the column. The eluate from the IMAC step was subjected to a size exclusion chromatography column equilibrated with 25 mM Tris/HCl pH 8, 0.15 M NaCl, 2 mM TCEP, 1% (v/v) βOG, 0.02% (w/v) NaN₃). ESI-MS confirmed the identity of CPT-II and showed that the protein was modified by amino-terminal α-N-gluconoylation (Geoghegan, K. F. et al. Spontaneous alpha-N-6-phosphogluconoylation of a “His tag” in Escherichia coli: the cause of extra mass of 258 or 178 Da in fusion proteins. Anal. Biochem., 267, 169-184 (1999)) (data not shown). The His-tag was not cleavable with thrombin. A 10 l fermentation (45 g of biomass) yielded 30 mg of electrophoretically pure, monomeric and monodisperse protein.

### Example 2

### Protein Crystallization

Crystals of apo CPT-2 and the ST1326 [(R)-N-tetradecylcarbamoyl-aminocarnitine] complex (in 33 mM stock solution in H₂O/NaOH) were obtained at a protein concentration of 15 mg/ml (in 25 mM Tris/HCl pH 8.0, 150 mM NaCl, 2 mM TCEP, 1% (w/v) n-octyl-β-D-glucopyronoside and 0.02% (w/v) NaN₃) and 21° C. using hanging drops with 25% (w/v) PEG 1500 or the modified microbatch (D'Arcy, A., et al., The advantages of using a modified microbatch method for rapid screening of protein crystallization conditions. Acta Cryst. D59, 396-399 (2003)) method with 0.15 M DL-malic acid pH 7.0, 20% (w/v) PEG 3350, respectively (Index 37 and 91, Hampton Research). For complex formation CPT-2 was incubated with a 3-fold molar excess of the inhibitor ST1326 for 3 h on ice prior to crystallization. The crystals were flash frozen in liquid nitrogen after 30 s soaks in mother liquor supplemented with 25% (w/v) PEG 200 (apo) or exchanging excess mother liquor and Al's oil against 100% (v/v) paraffin oil (ST1326).

### Example 3

### Data Collection and Processing

Datasets for apo CPT-2 and the ST1326 complex were collected on beam line X10SA at SLS, Villigen, Switzerland. The measurements were performed at 100 K with λ=0.97853 Å (apo, Se peak wavelength) and λ=1.008 Å (ST1326). The datasets were processed and scaled with XDS (Kabsch, W. Automatic processing of rotation diffraction data from crystals of initially unknown symmetry and cell constants. J. Appl. Cryst., 26, 795-800 (1993)). The space group was determined as P4₃2₁2 (apo tetragonal, a=b=67.6, c=307.3, α=β=γ=90°, Matthew's Coeff.=2.4 (Matthews, B. W. J. Mol. Biol., 33, 491-497 (1968))), C222₁ (apo orthorhombic, a=95.2, b=97.3, c=310.4, Matthew's Coeff.=2.4) and P2₁2₁2₁ (ST1326, a=85.8, b=96.2, c=124.3, α=β=γ=90°, Matthew's Coeff.=3.5). First crystals of apo CPT-2 diffracted to 1.9 Å resolution but the space group could not be unequivocally assigned. In contrast, apo crystals obtained from material that was seleno-methionine labeled by standard procedures (Chene, C. et al. Crystallization of the complex of human IFN-gamma and the extracellular domain of the IFN-gamma receptor. Proteins, 23, 591-594 (1995)) were of superior diffraction quality, which could be attributed to improved protein quality due to the different media composition and bacterial protein expression profile during labeling.

The XDS rejection statistics report 0.26% rejected misfits which fully supports the choice of P2₁2₁2₁ as the correct space group for the complex crystal despite the rather high value for R_{merge} of 16.7%. Data statistics are summarized in Table 1. Both crystal forms contain one molecule of CPT-2 per asymmetric unit.

Structure solution and refinement. Molecular replacement was performed using AMoRe in the CCP4 interface (Navazza, J. AMoRe: a new package for molecular replacement, in Molecular replacement. Proceedings of the CCP4 Study Weekend, 87-90, Daresbury Laboratory, Warrington, England (1992); Collaborative Computational Project, Number 4. The CCP4 suite: Programs for protein crystallography. Acta Cryst., D50, 760-763 (1994)). For the ST1326 complex a rat CPT-2 homology model built with XSAE (C. Broger, F. Hoffmann La-Roche) based on mouse CrAT (Hsiao, Y. S., et al., Structural and biochemical studies of the substrate selectivity of carnitine acetyltransferase. J. Biol. Chem., 279, 31584-31589 (2004)) (PDB code 1t7n) served as search model. The solution was refined by simulated annealing with CNS (Brünger, A. T. X-PLOR Manual Version 3.1. New Haven, Conn., USA, Yale University Press (1992)). The final model was built using iterative cycles of model building in MOLOC (Gerber, P. R. Peptidemechanics: a force field for peptides and proteins working with entire residues as small units. Biopolymers, 32, 1003-1017 (1992)), solvent building in autoBUSTER (Roversi, P., et al., Modelling prior distributions of atoms for macromolecular refinement and completion. Acta Cryst., D 56, 1316-1323 (2000)) and refinement in Refmac (Murshudov, G. N., Vagin, A. A., Lebedev, A., Wilson, K. S. & Dodson, E. J. Efficient anisotropic refinement of macromolecular structures using FFT. Acta Cryst., D 55, 247-255 (1999)). Despite Se-labeling the apo structure was also solved by molecular replacement using the refined complex structure as search model, followed by automated model building with Arp/wArp (Perrakis, A., et al.,. Automated protein model building combined with iterative structure refinement. Nat. Struct. Biol., 6, 458-463 (1999)) and iterative Refmac and MOLOC cycles. For the orthorhombic (tetragonal) apo structure 94.0% (92.7%) of the residues lie in the most favored and 5.4% (6.8%) in the additionally allowed regions of the Ramachandran Plot. For the ST1326 complex structure these values are 88.2% and 11.3%, respectively. Arg 498 (generously allowed region) and Leu 129 as well as Asn 230 (Ramachandran outliers) account for the differences to 100%.

Refinement statistics are summarized in Table 1. Figures with molecule representations were made with PyMol (DeLano, W. L. The PyMOL User's Manual, DeLano Scientific, San Carlos, Calif., USA (2002)) and MOE (Chemical Computing Group Inc., MOE, 1010 Sherbrooke Street West, Suite 910, Montreal, Canada, H3A 2R7).

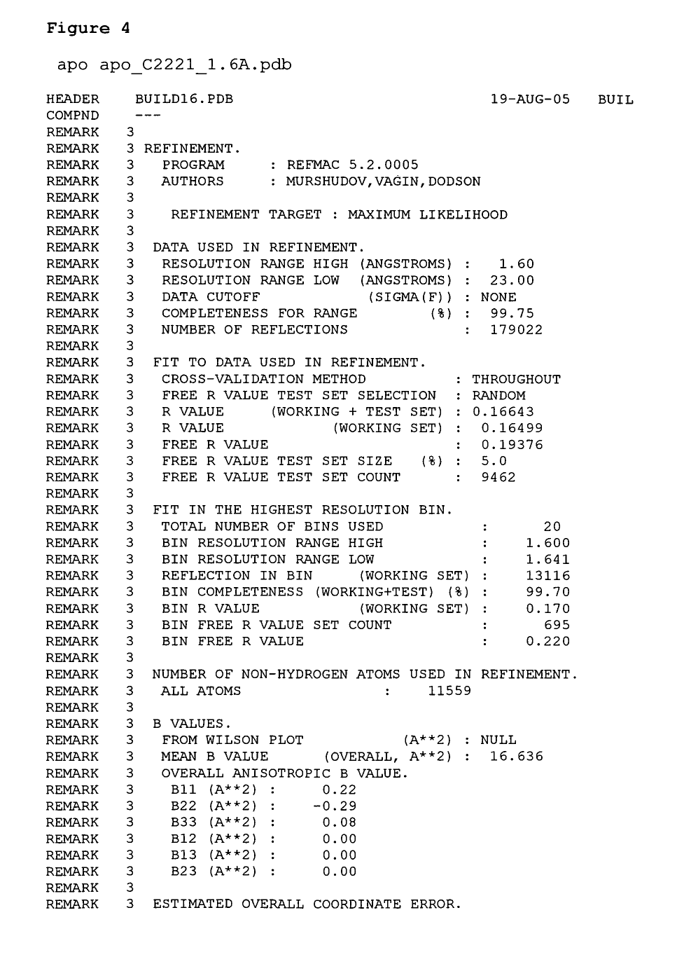

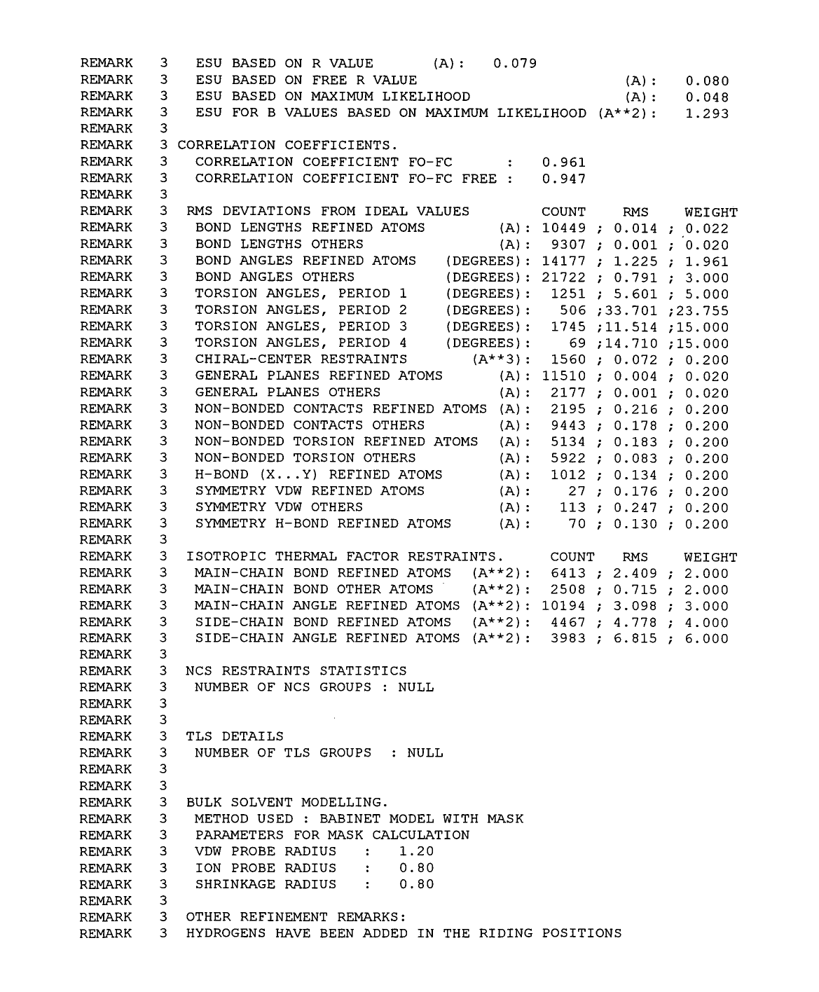

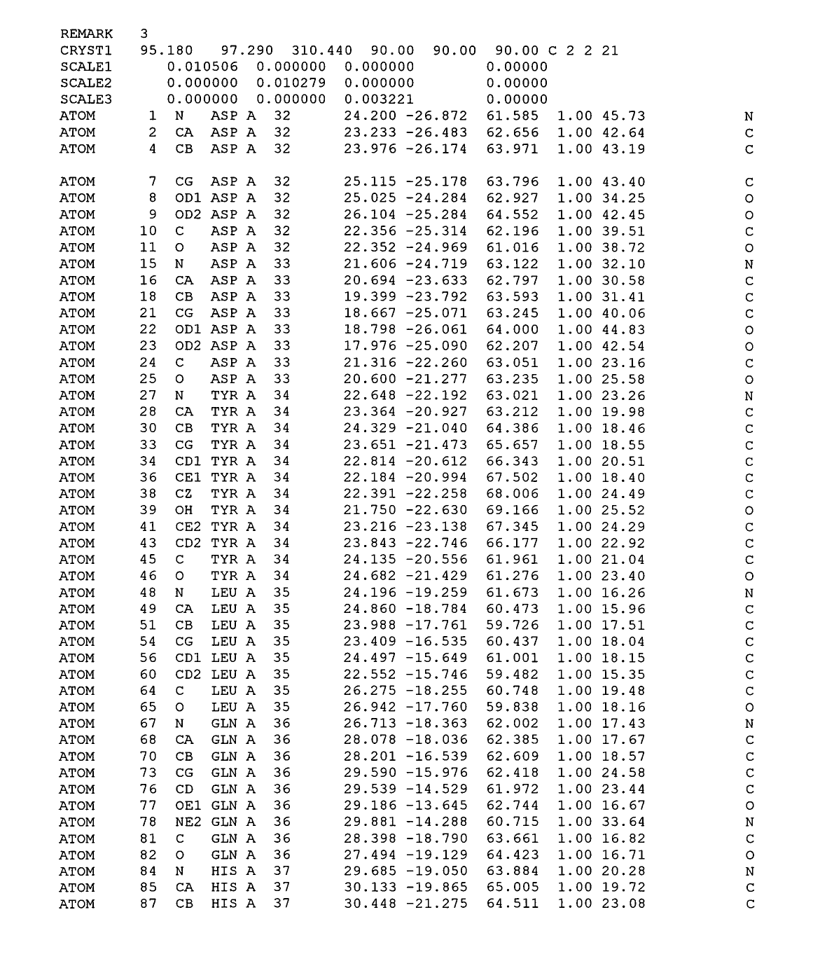

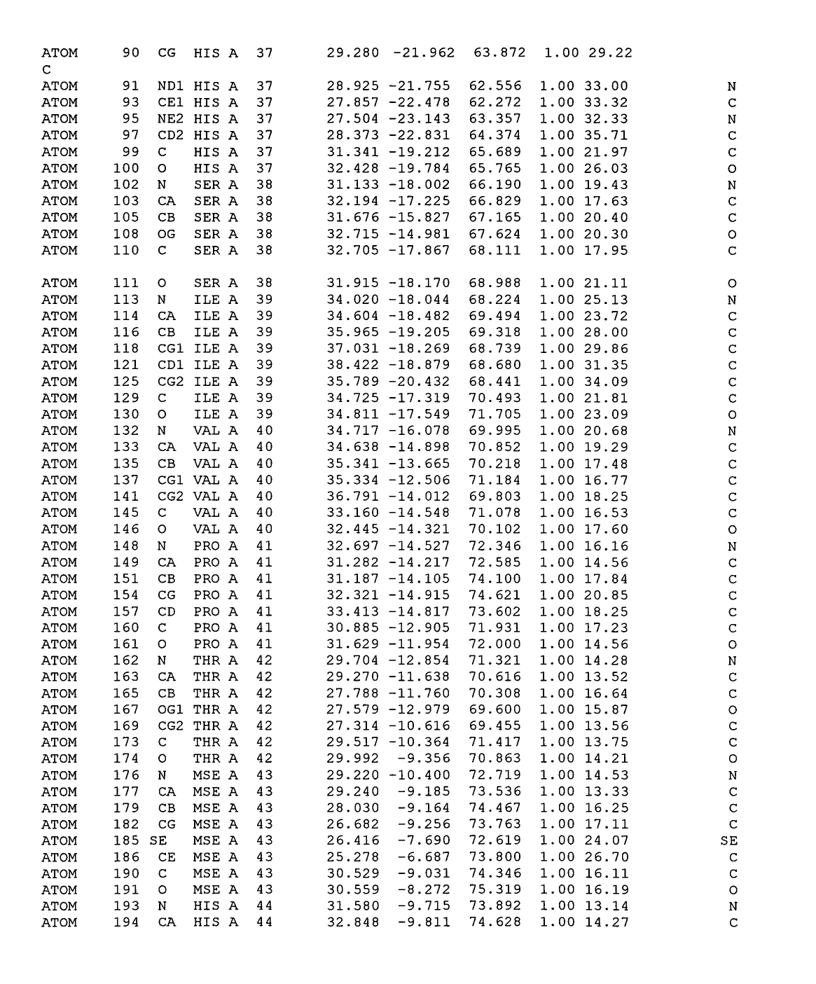

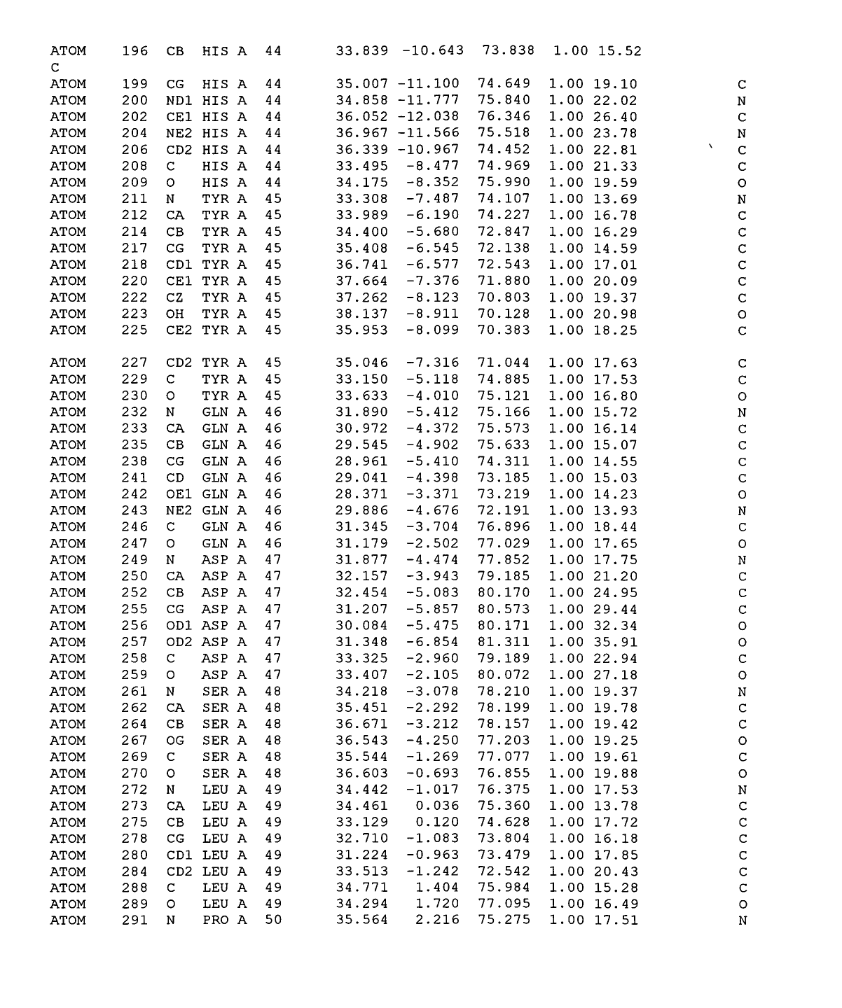

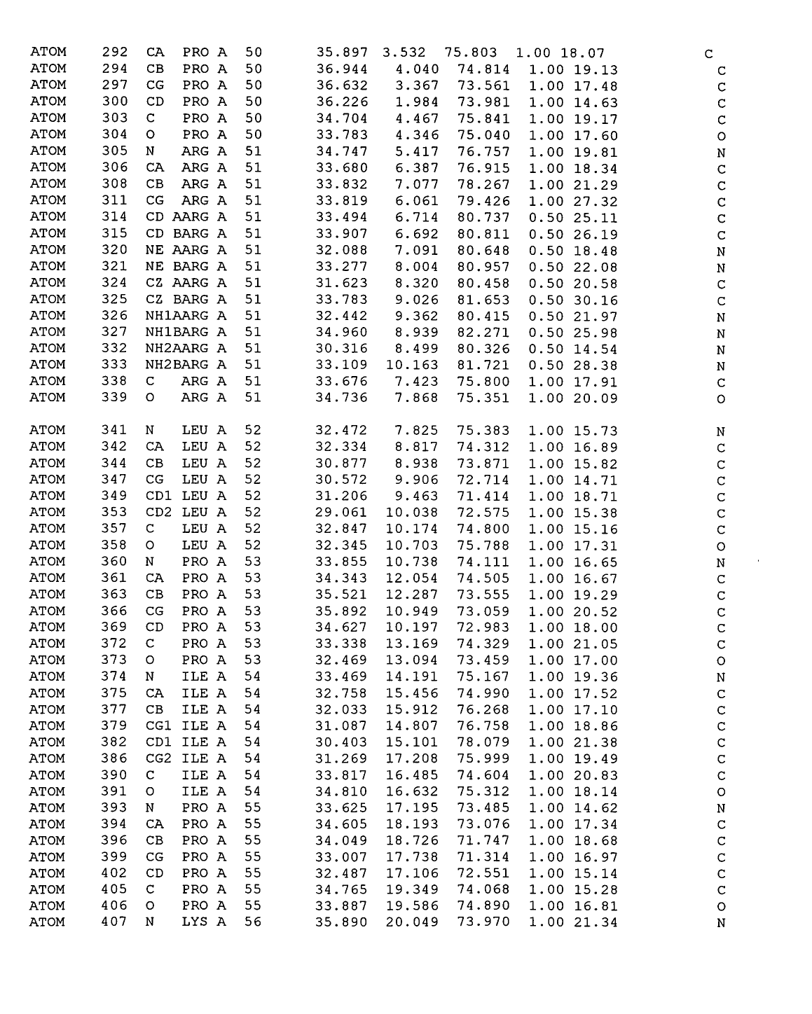

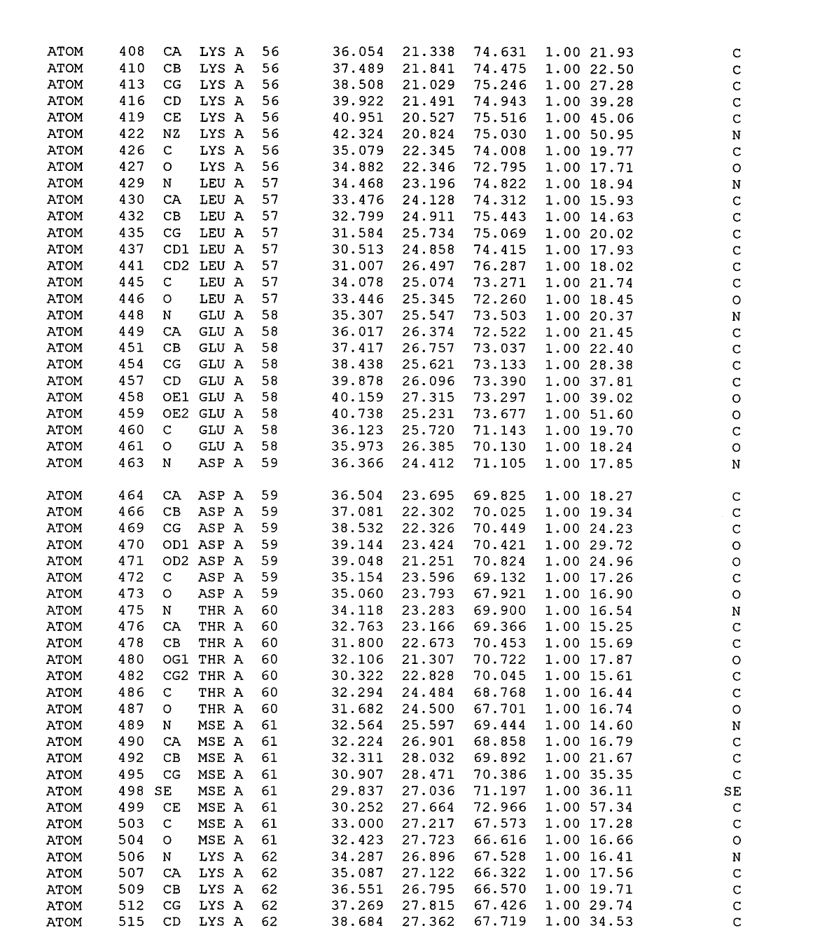

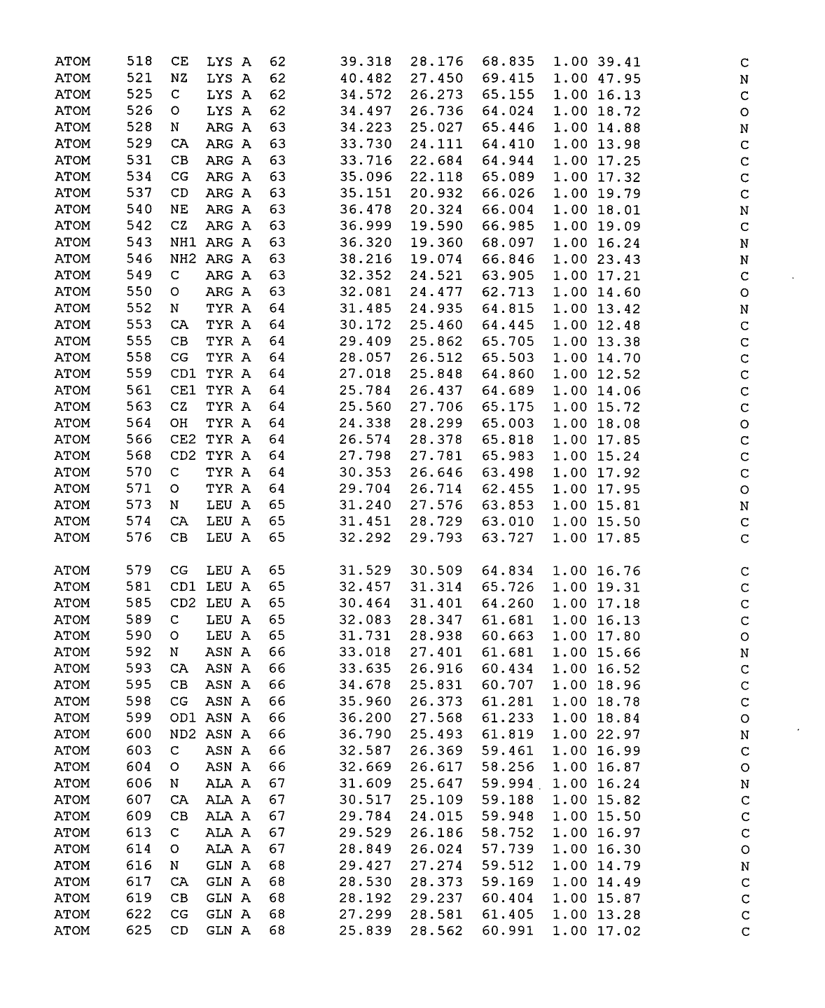

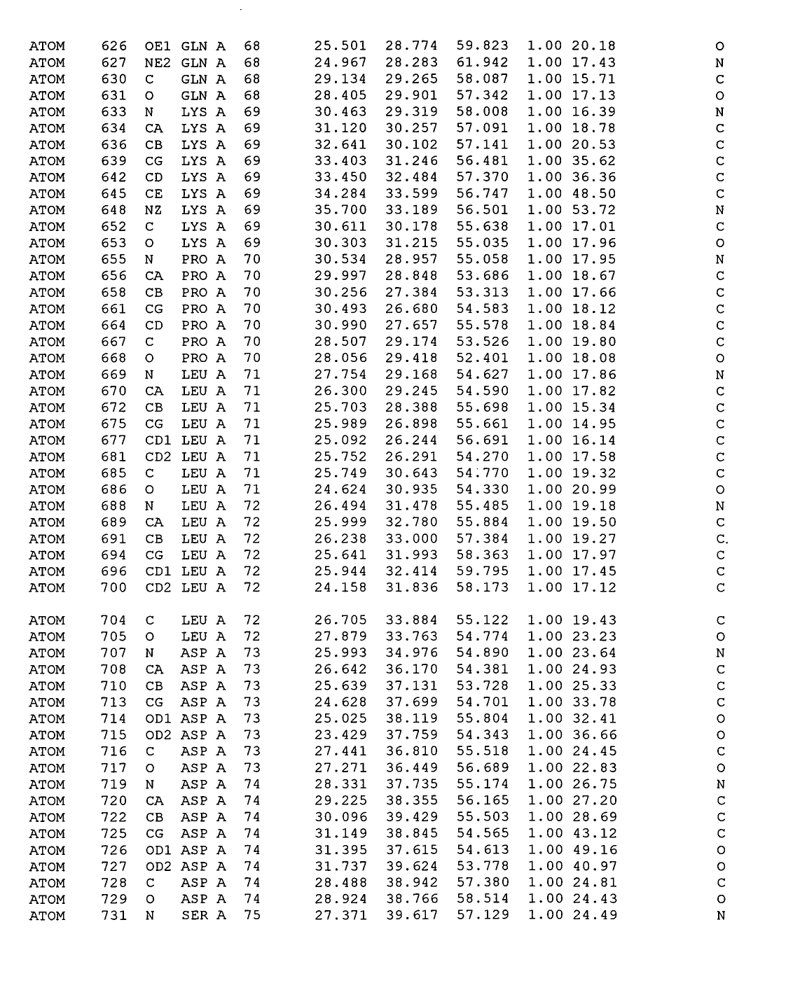

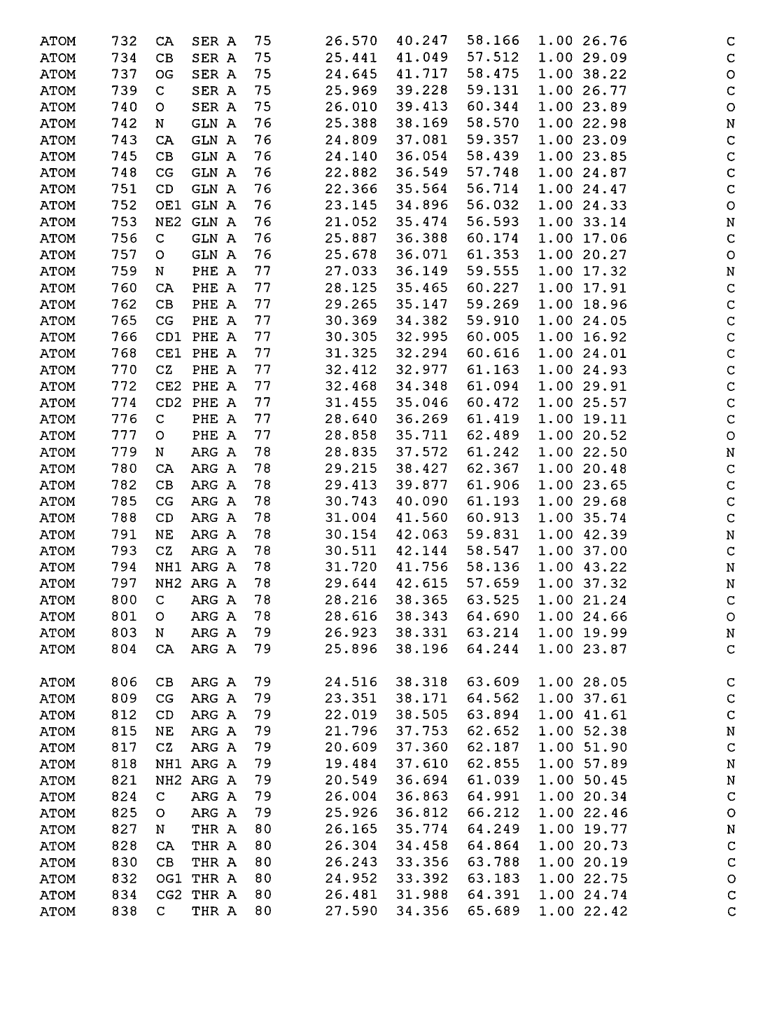

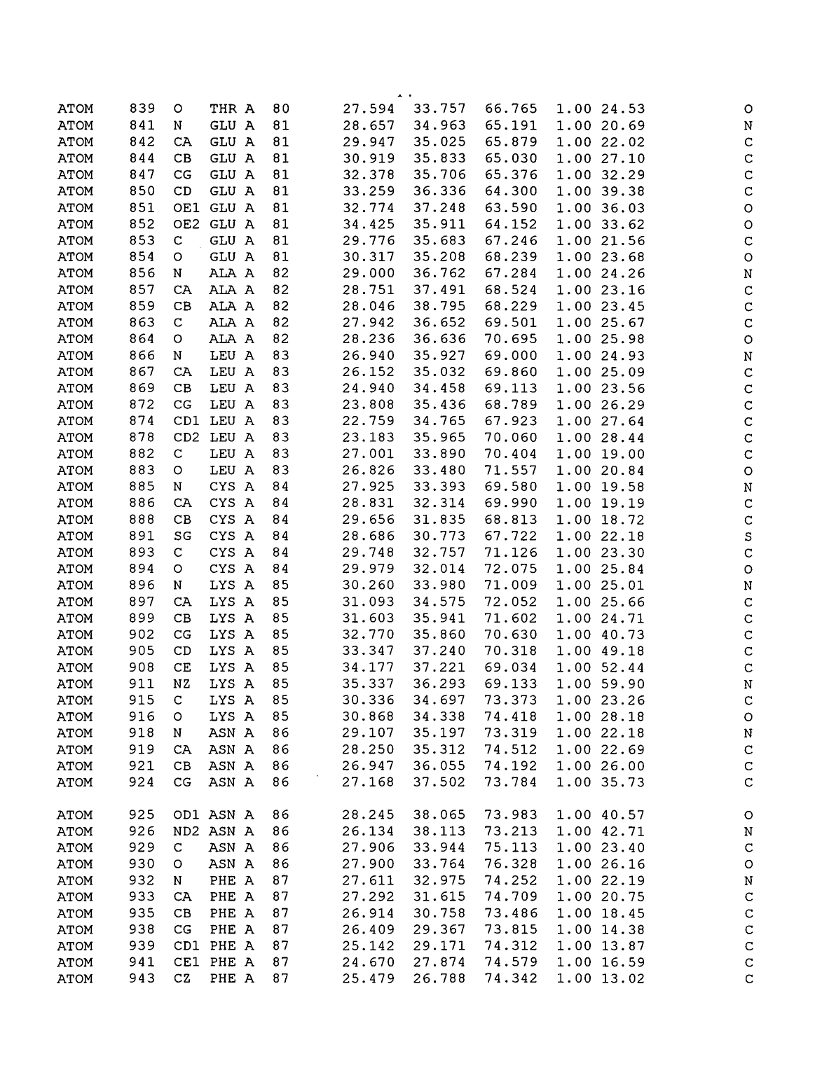

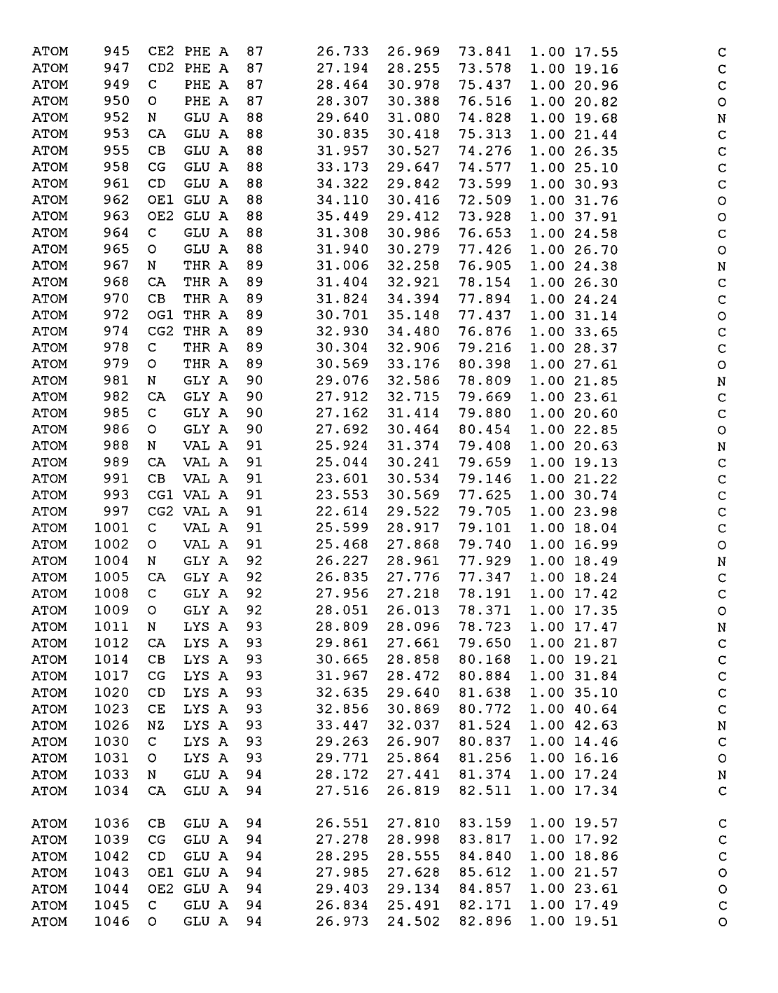

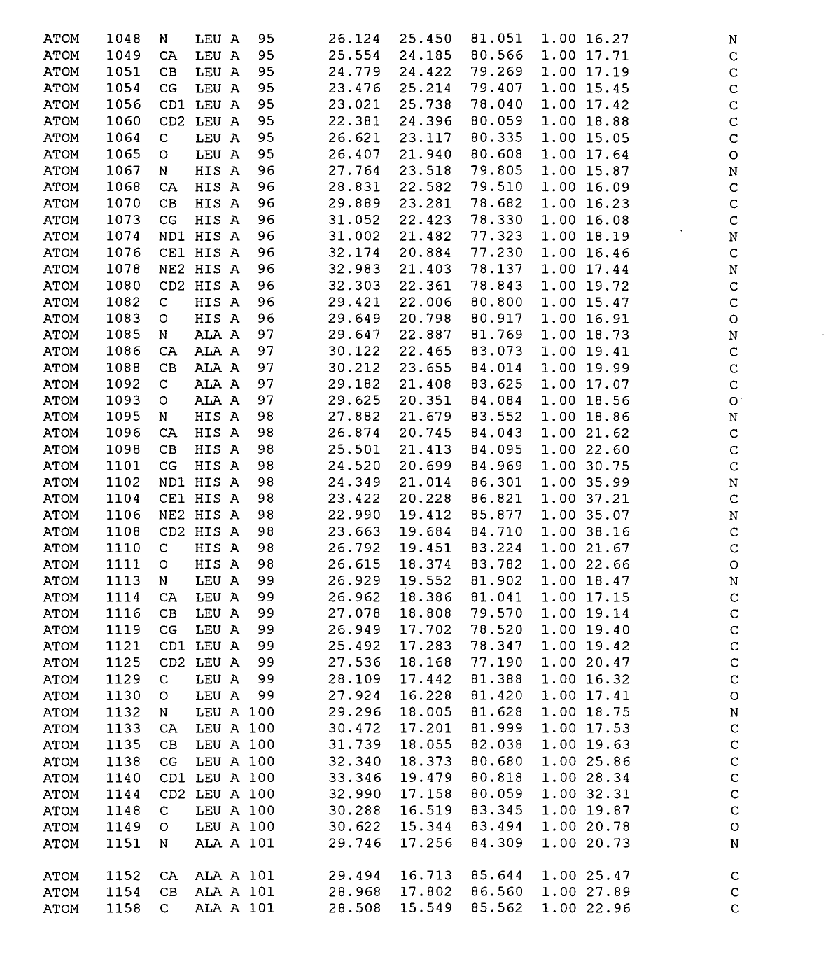

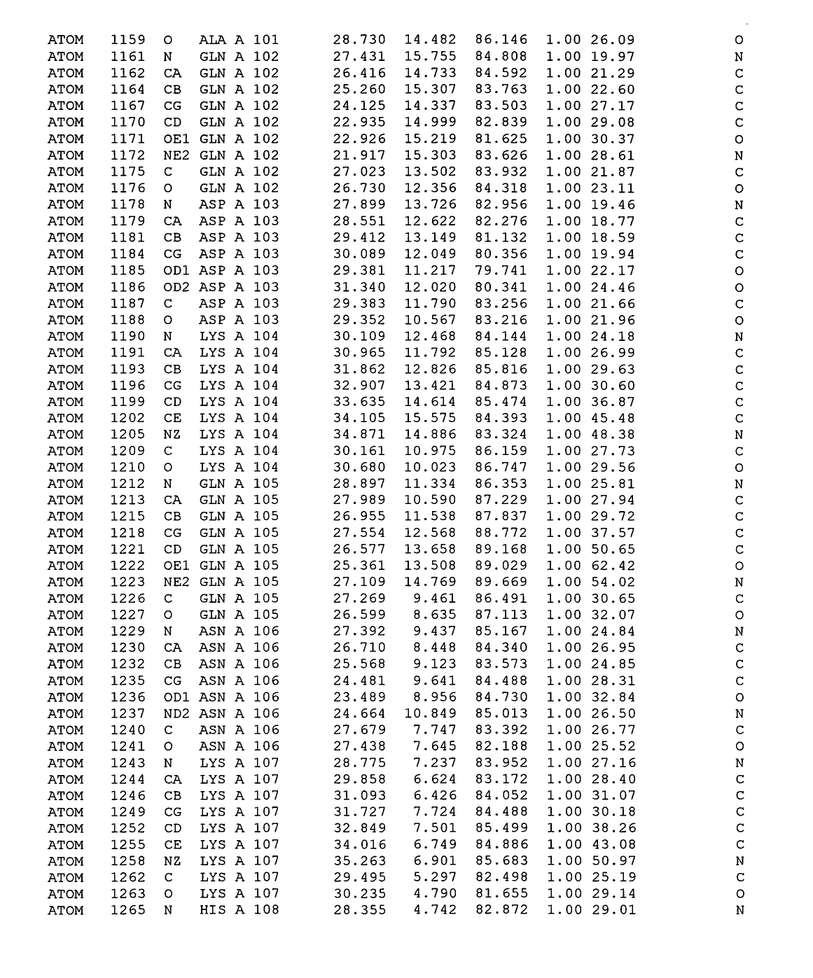

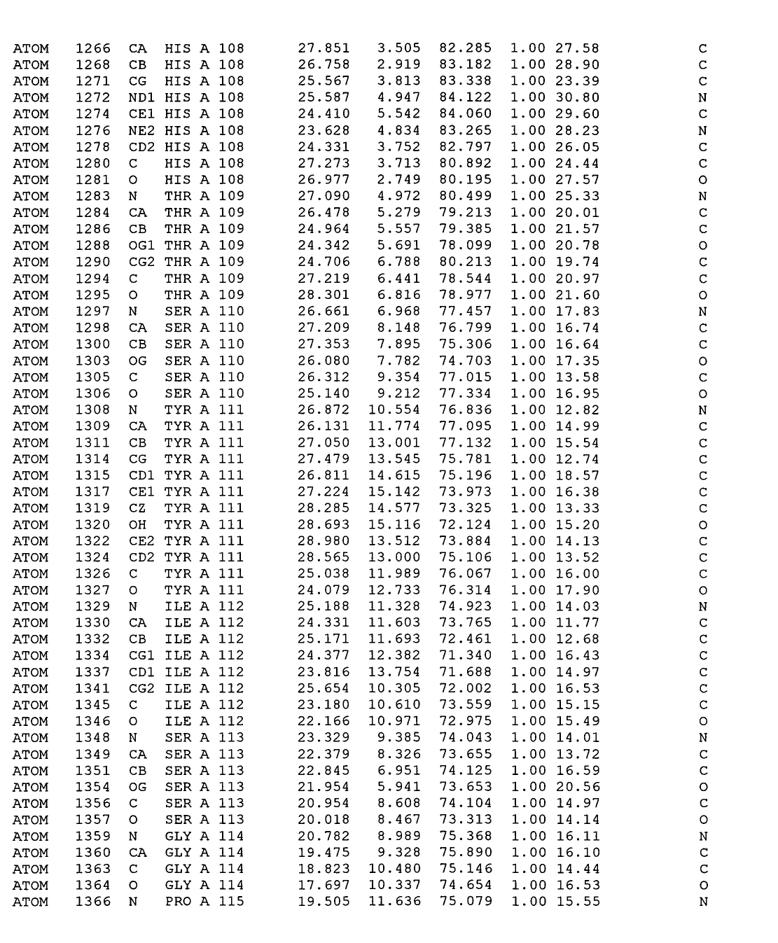

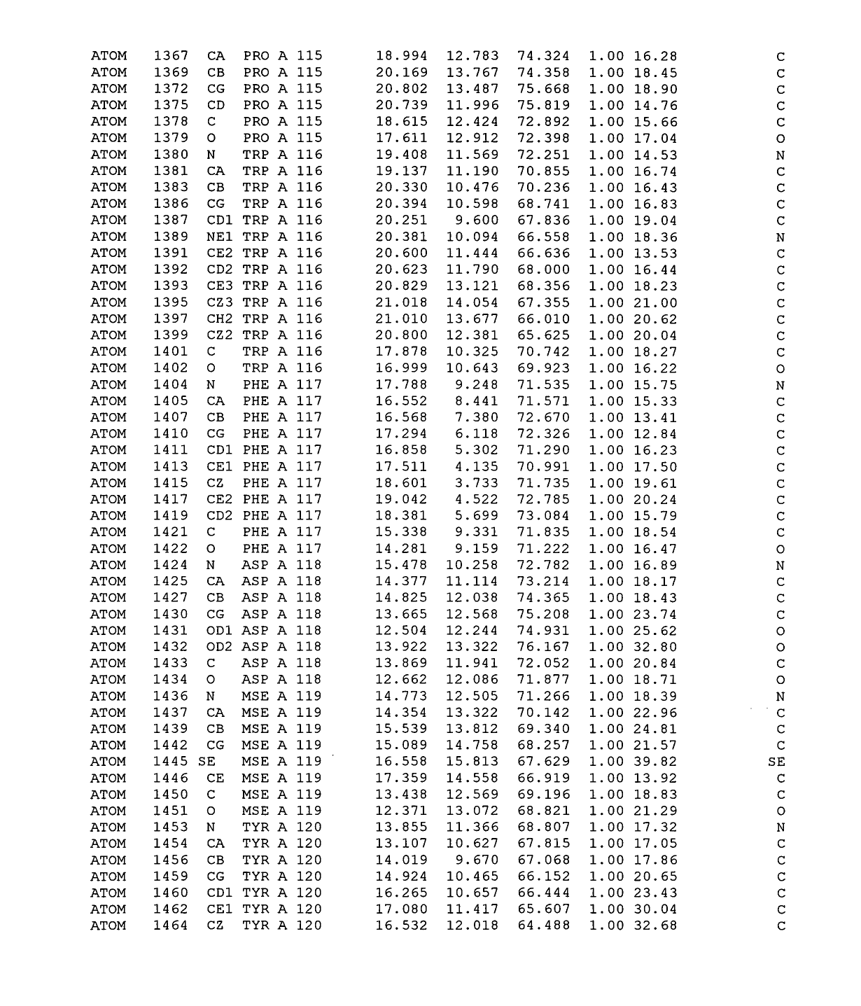

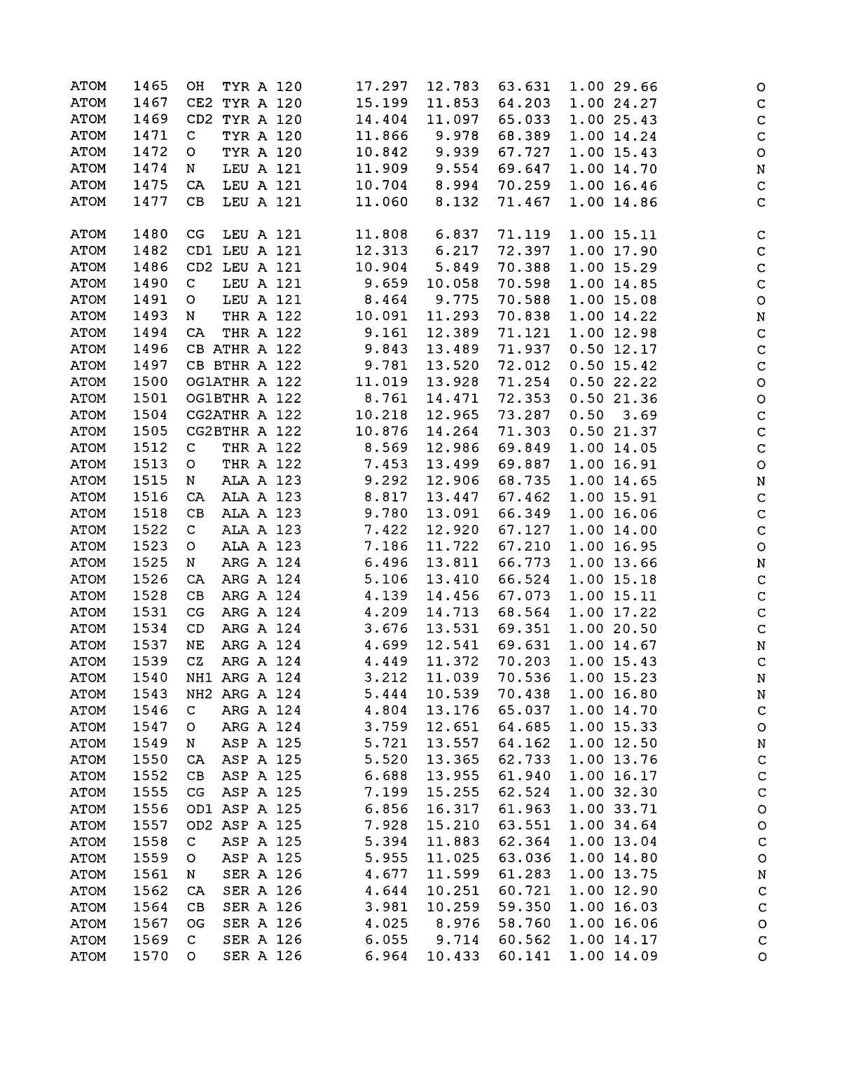

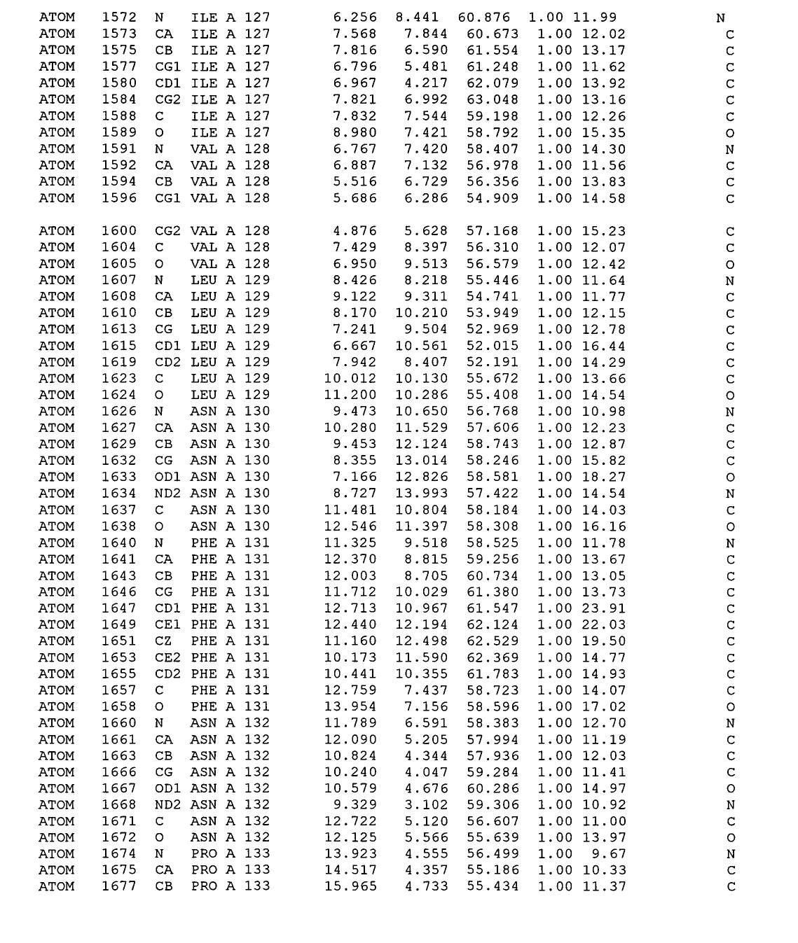

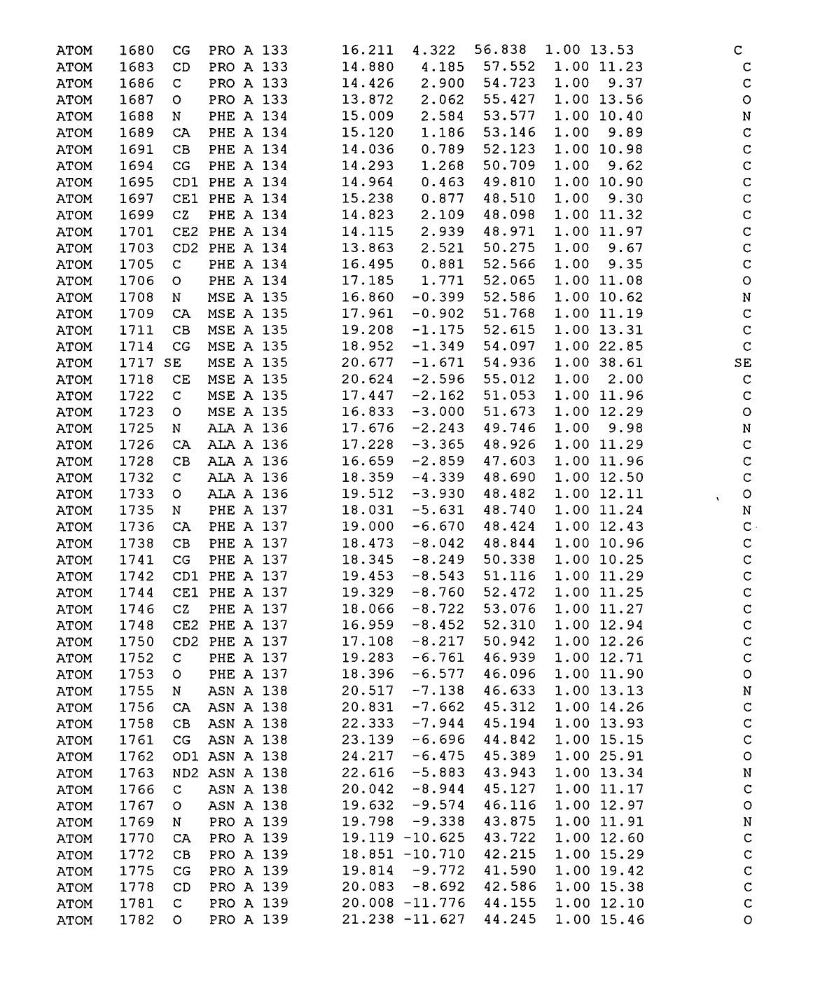

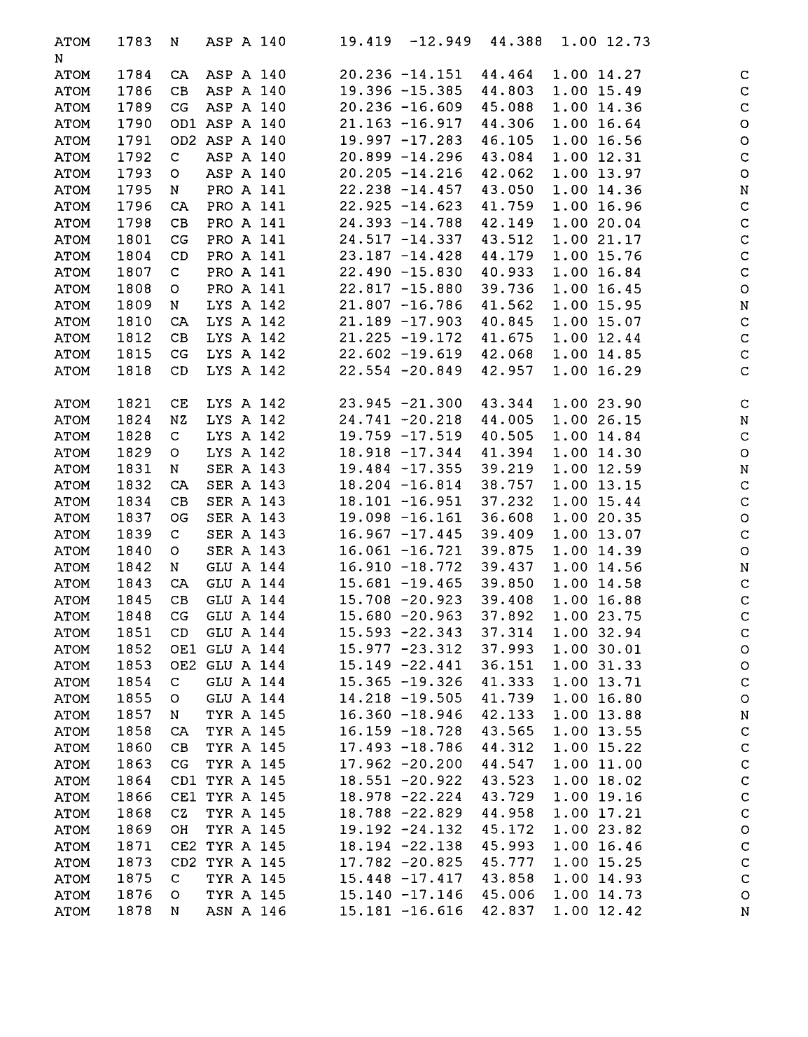

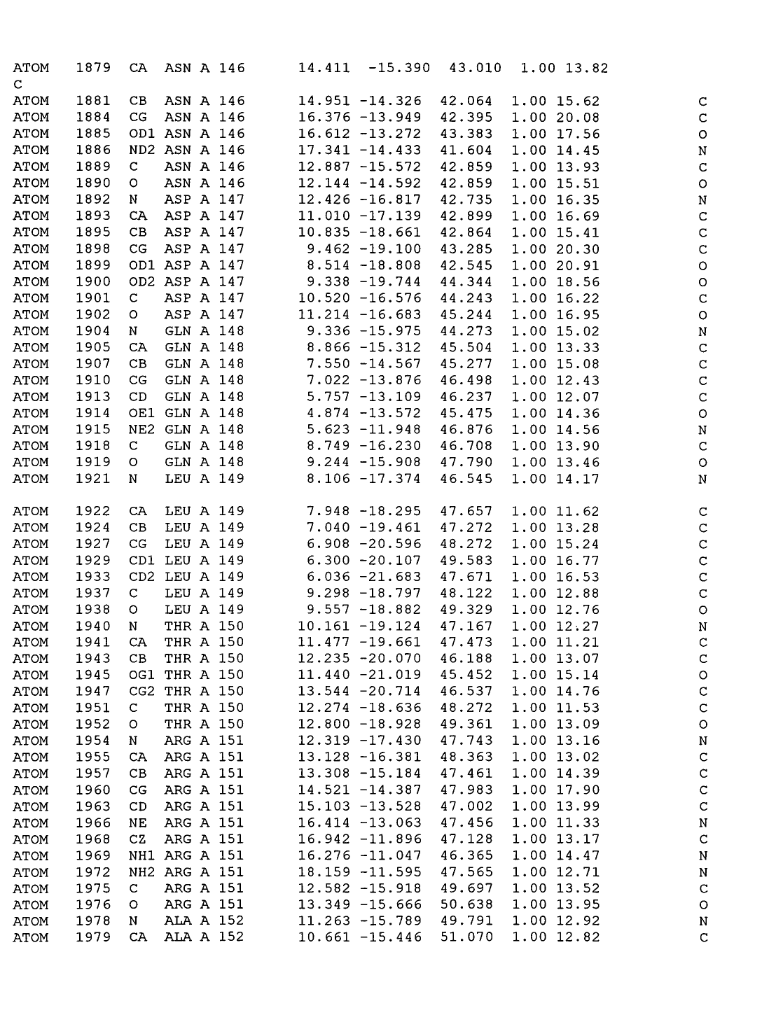

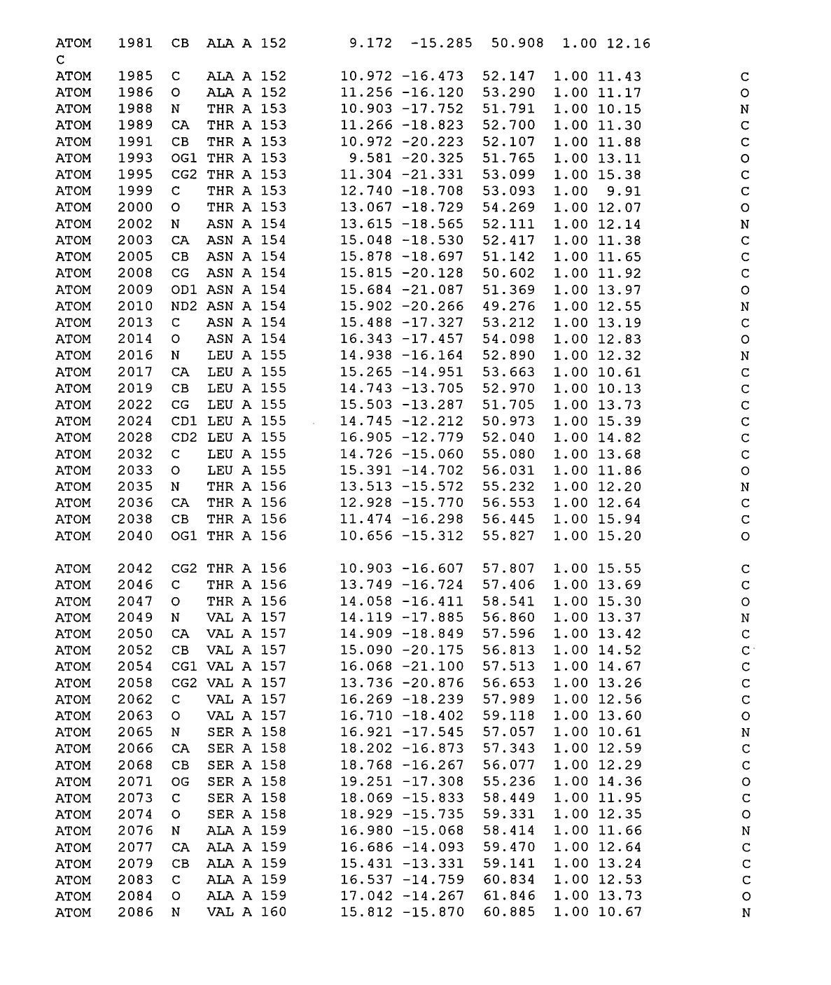

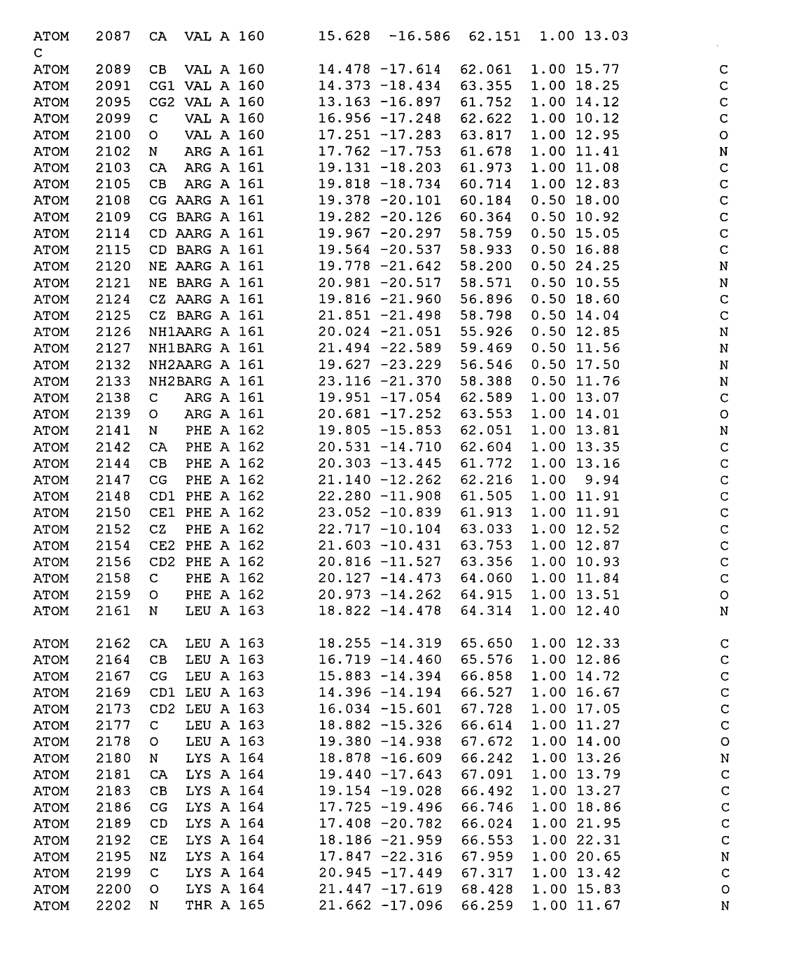

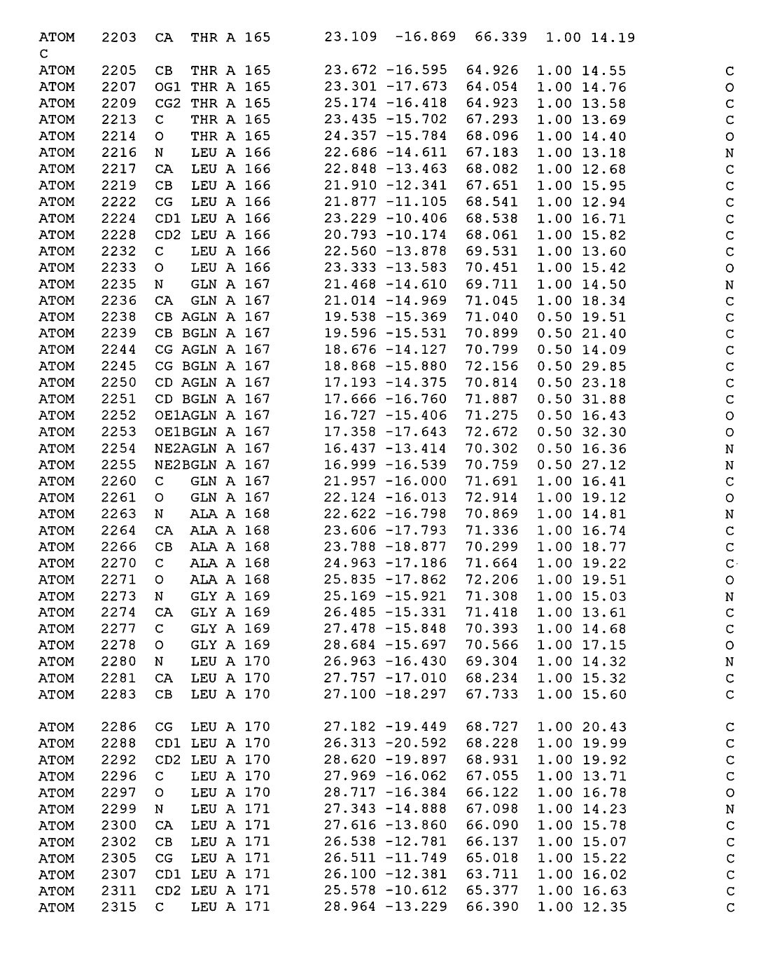

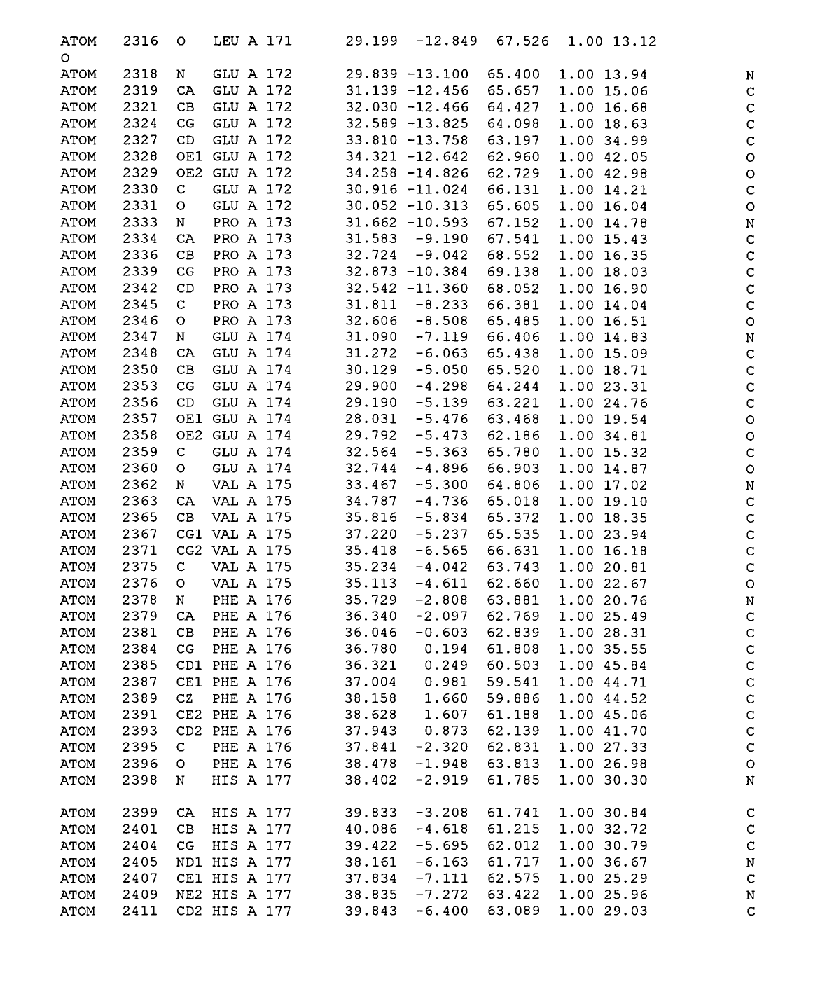

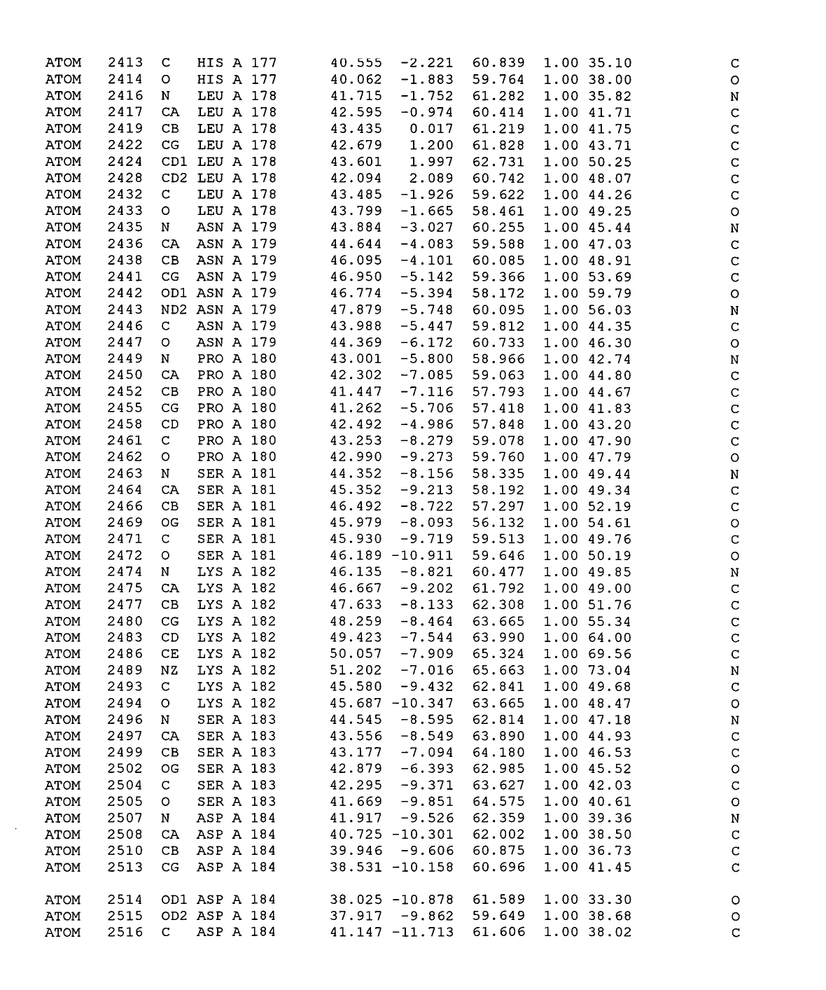

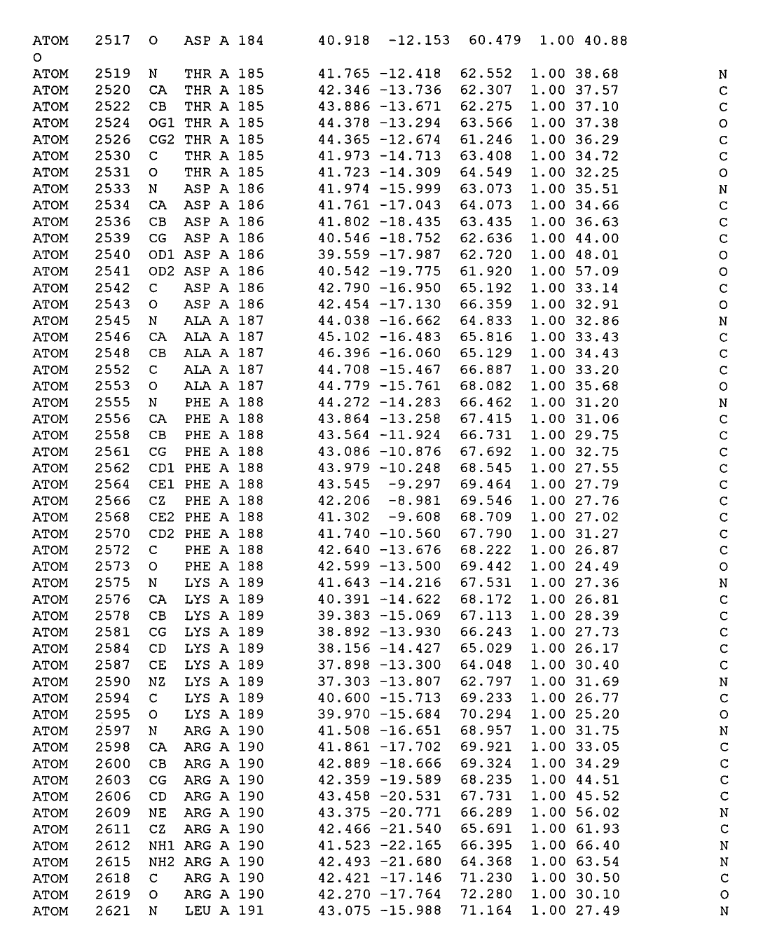

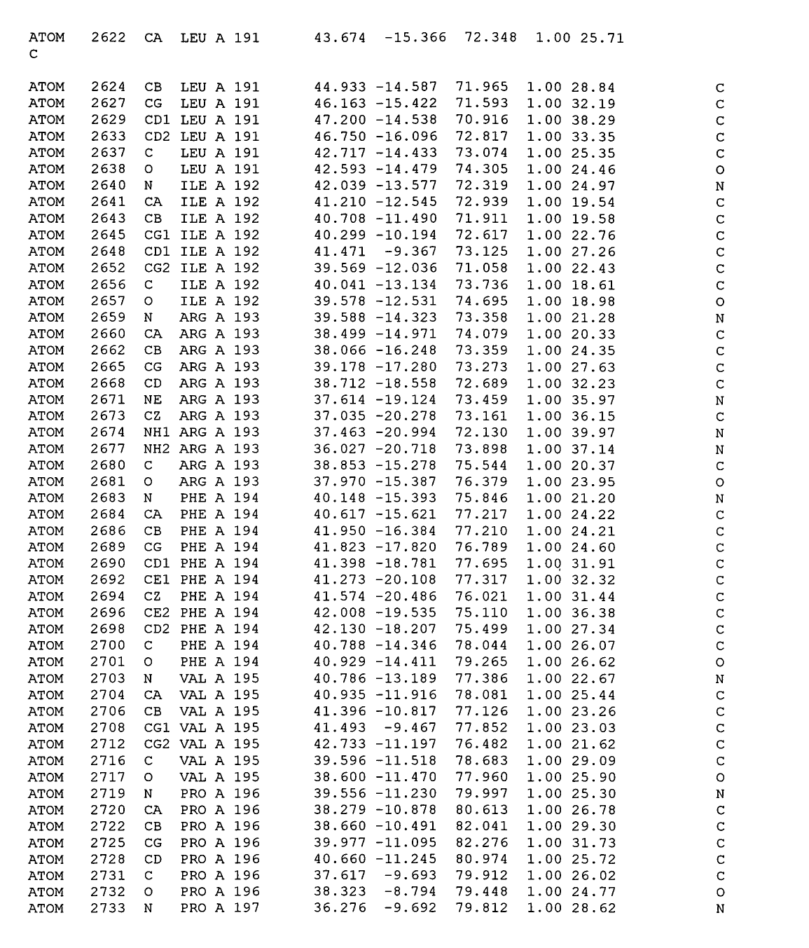

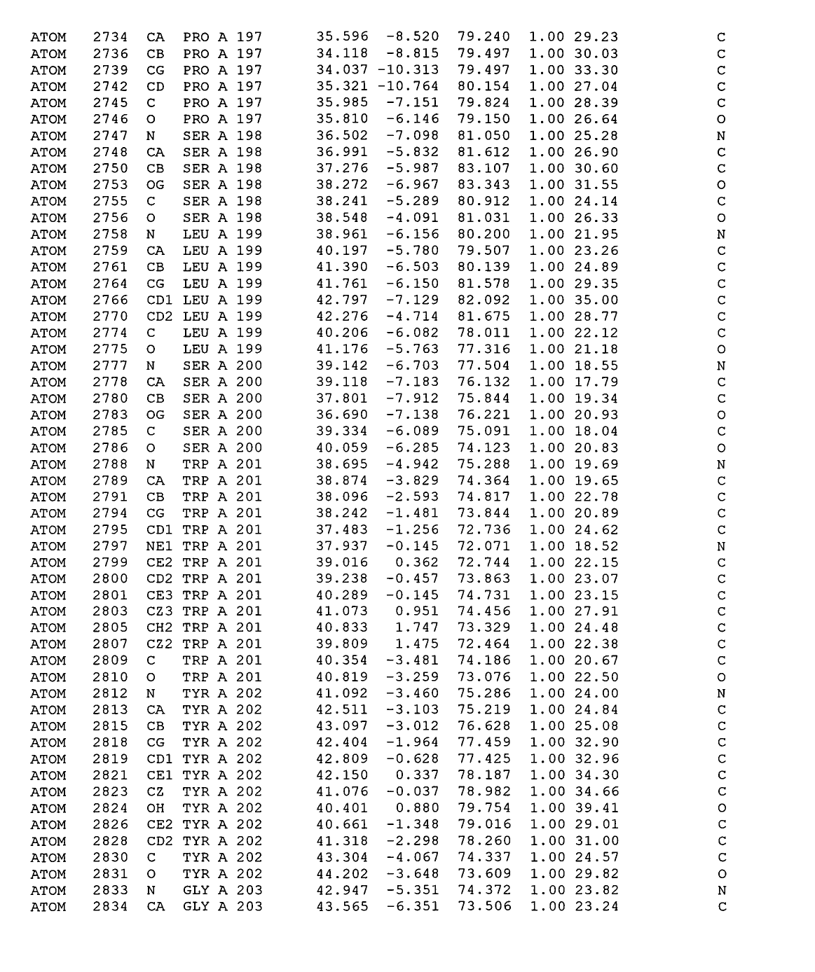

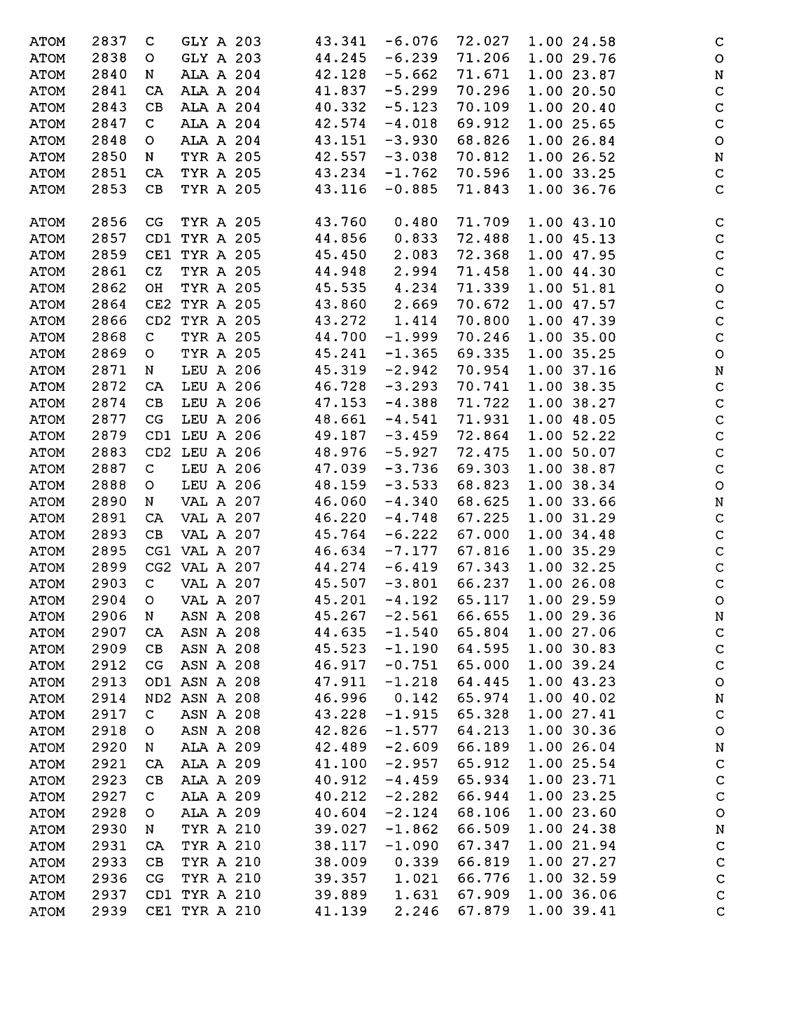

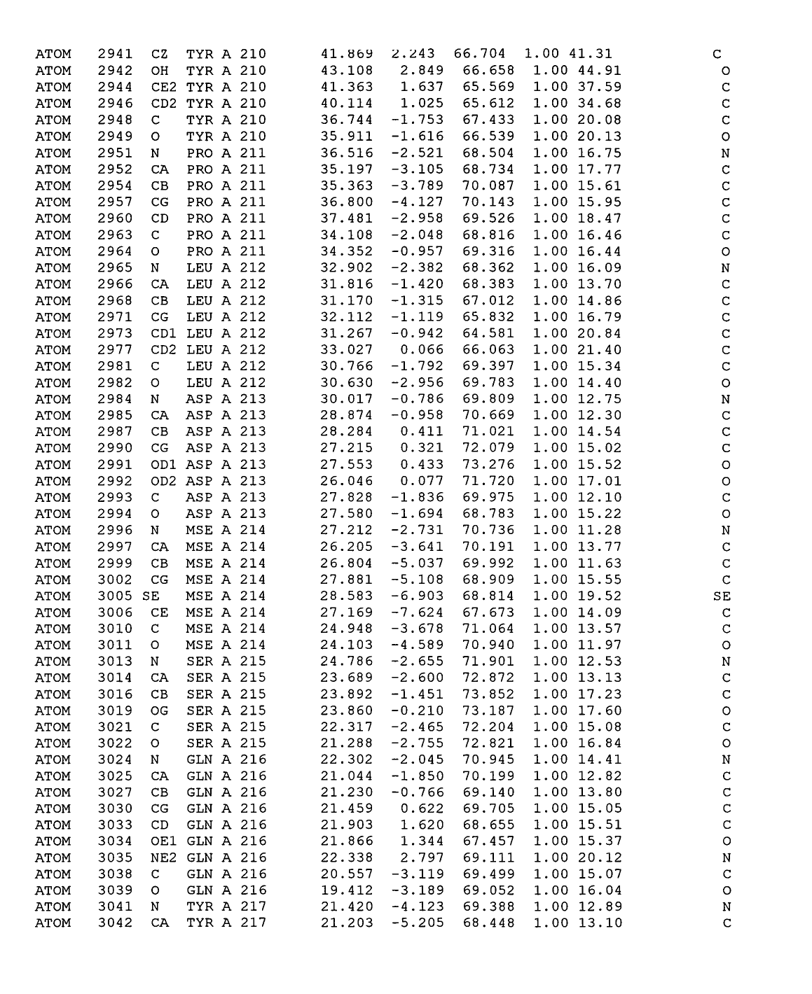

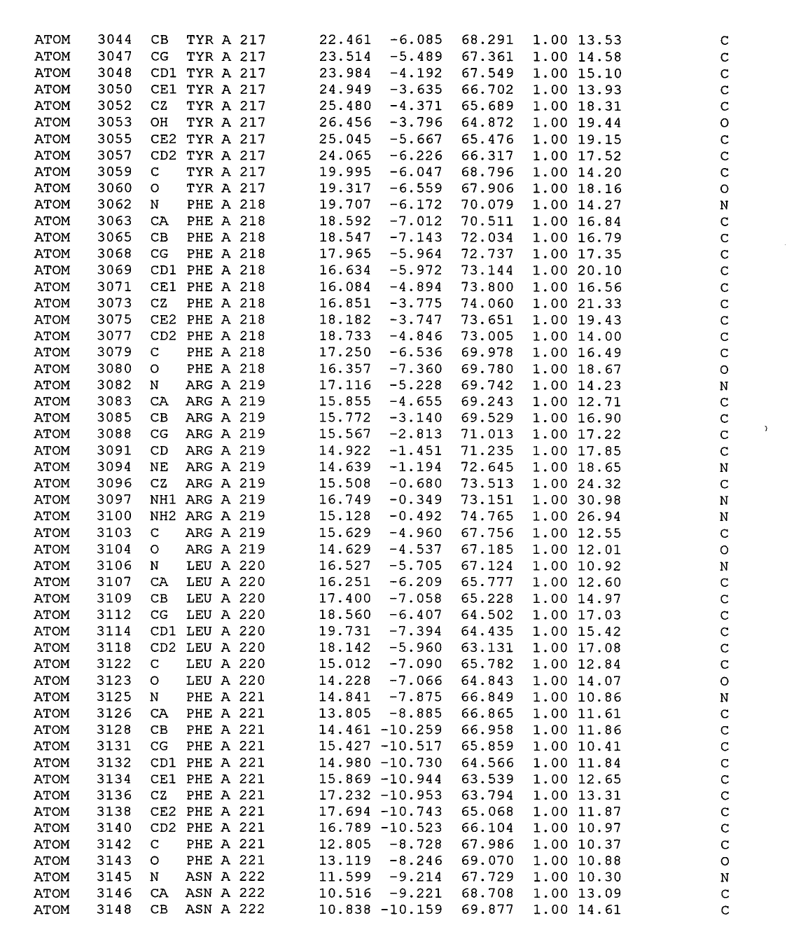

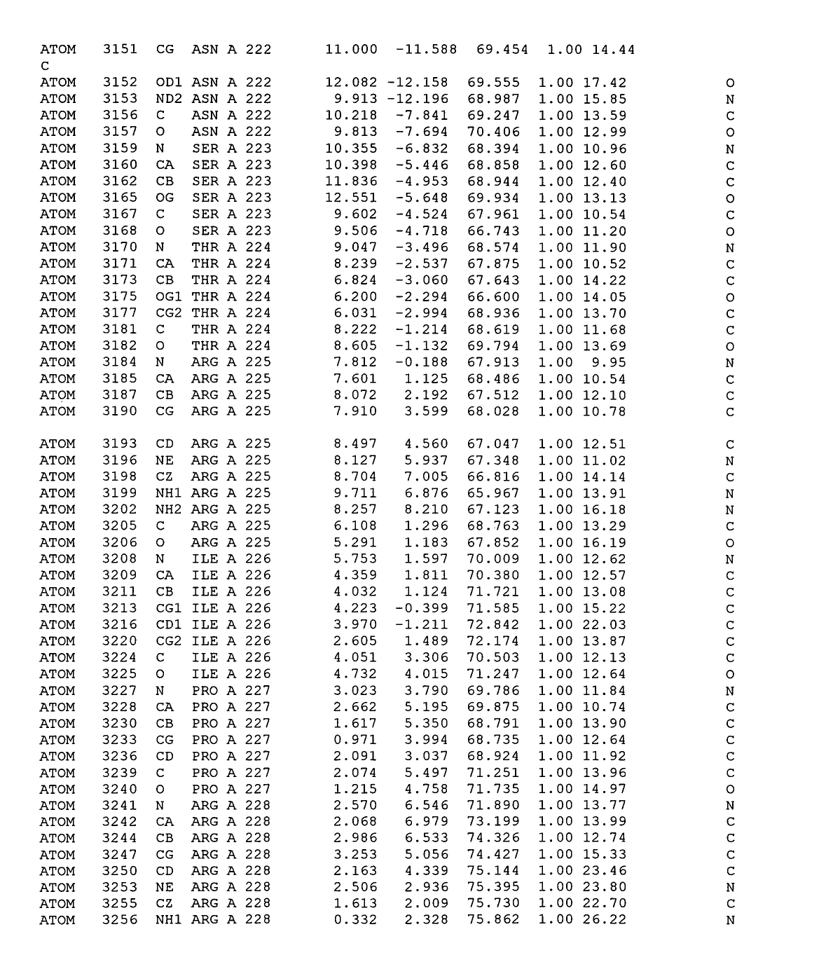

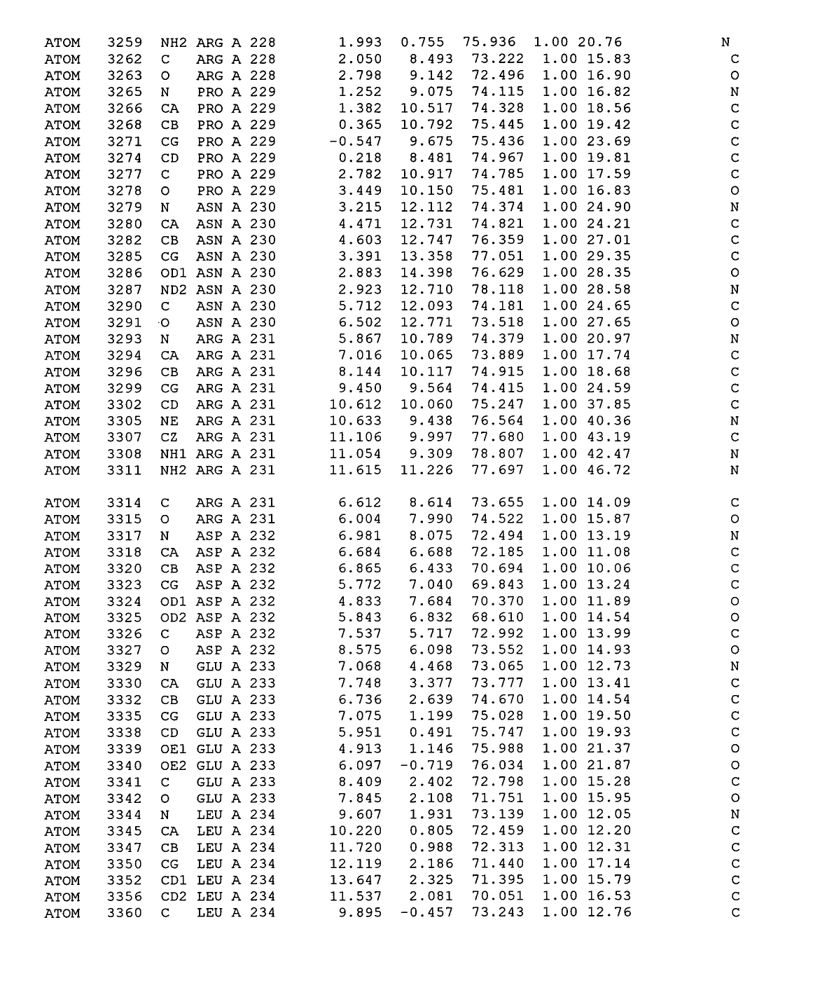

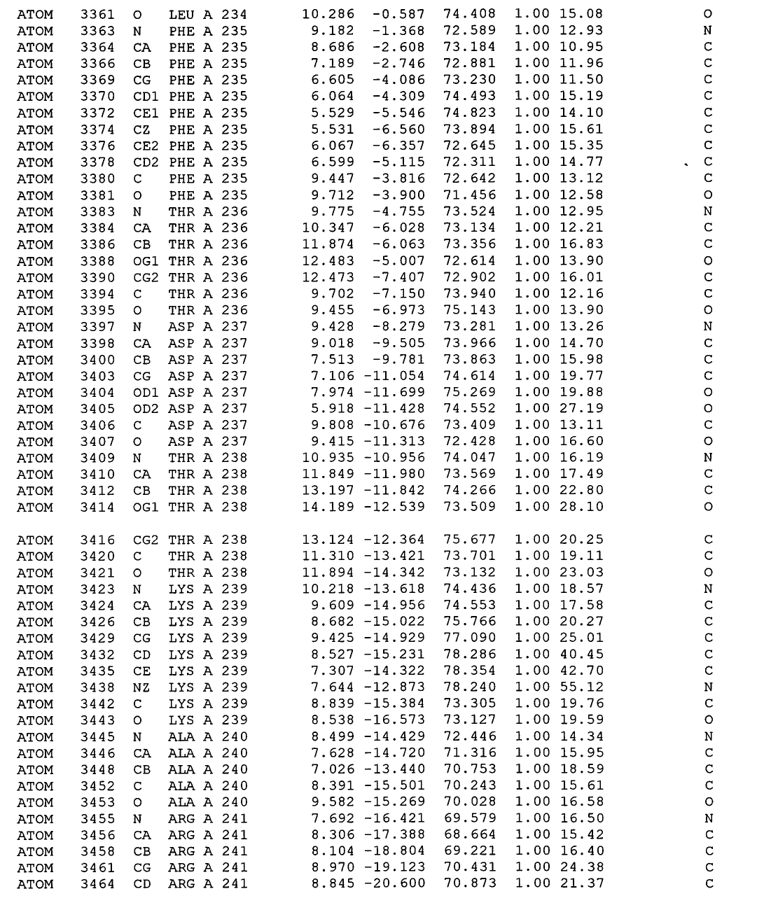

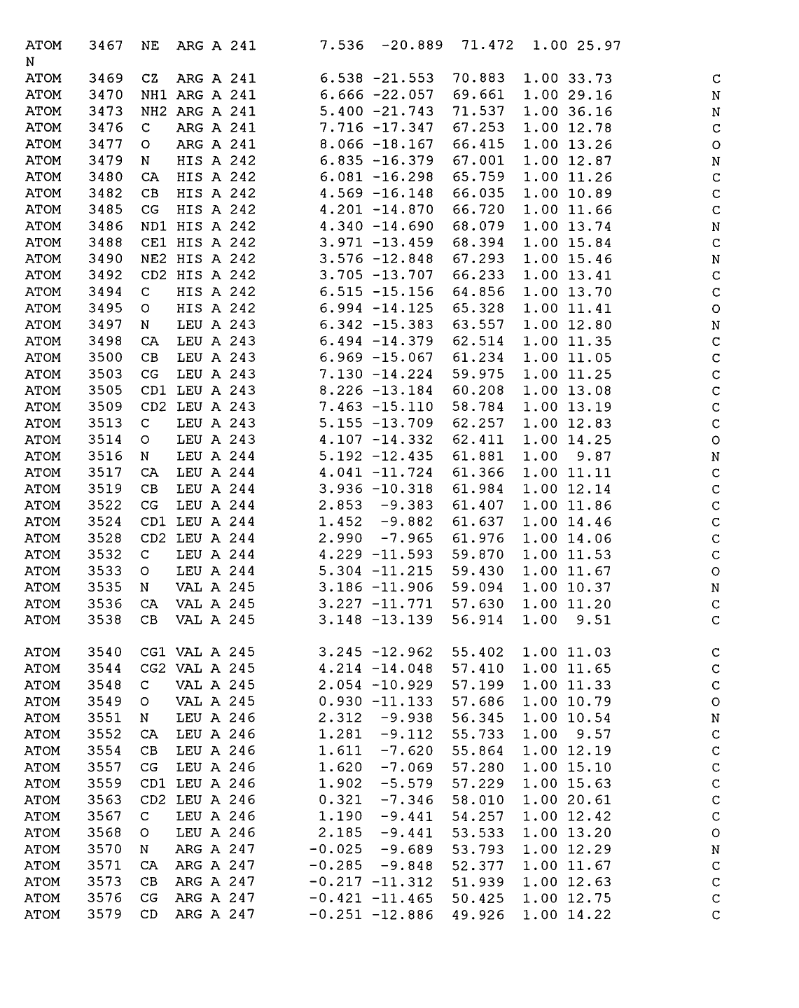

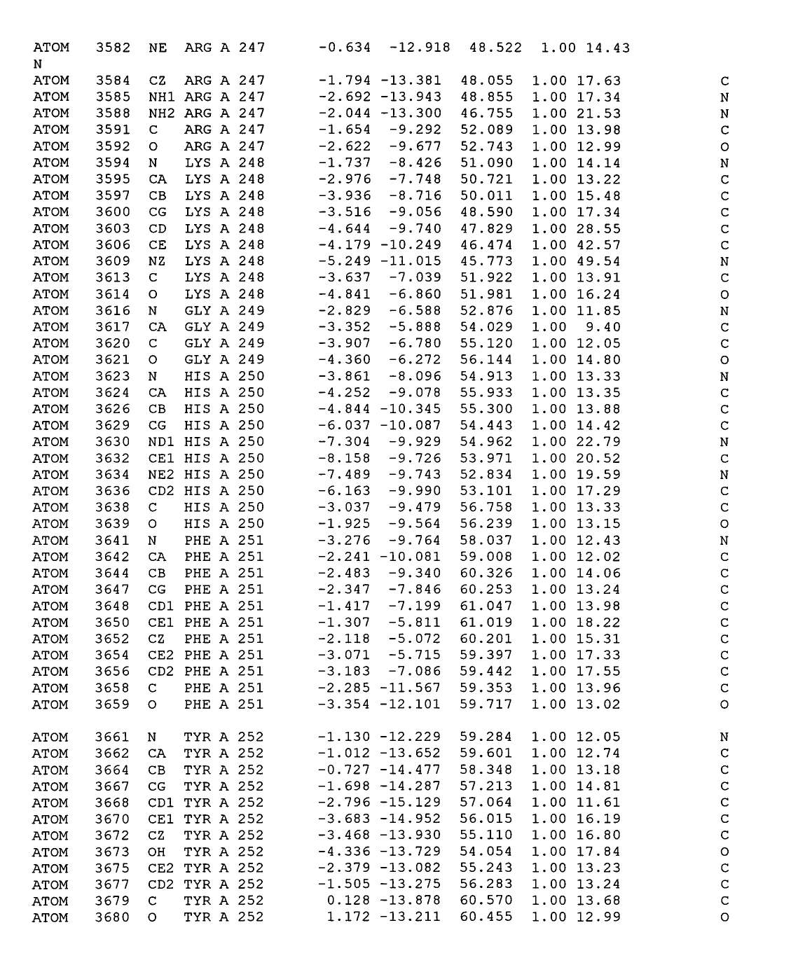

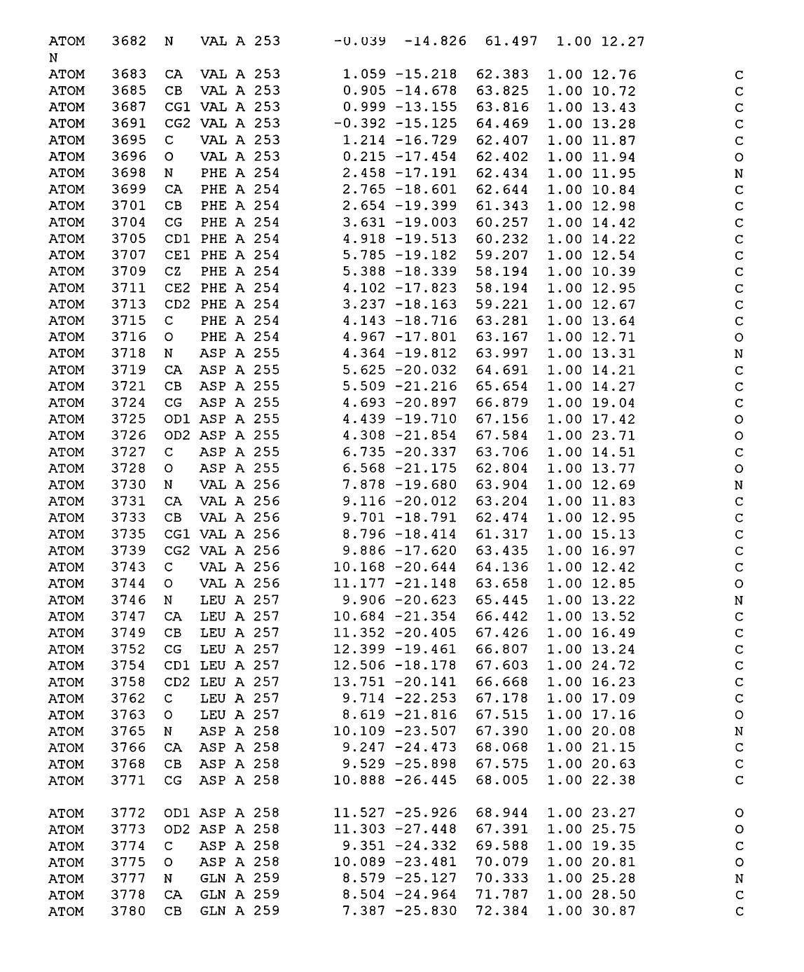

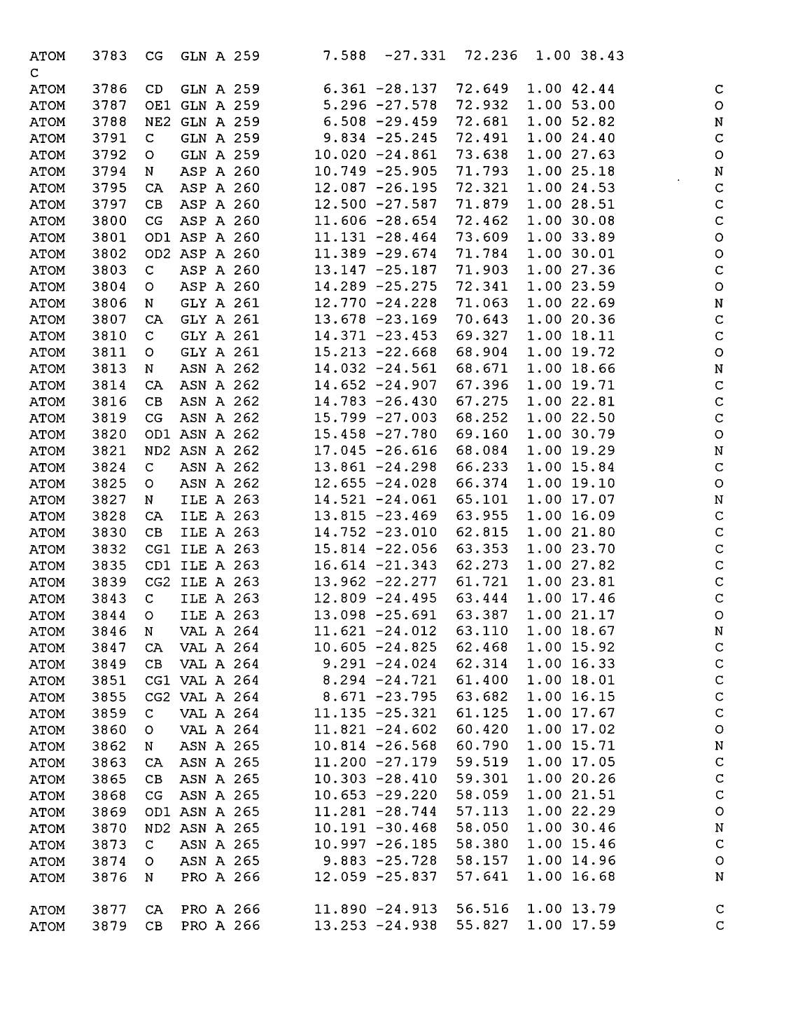

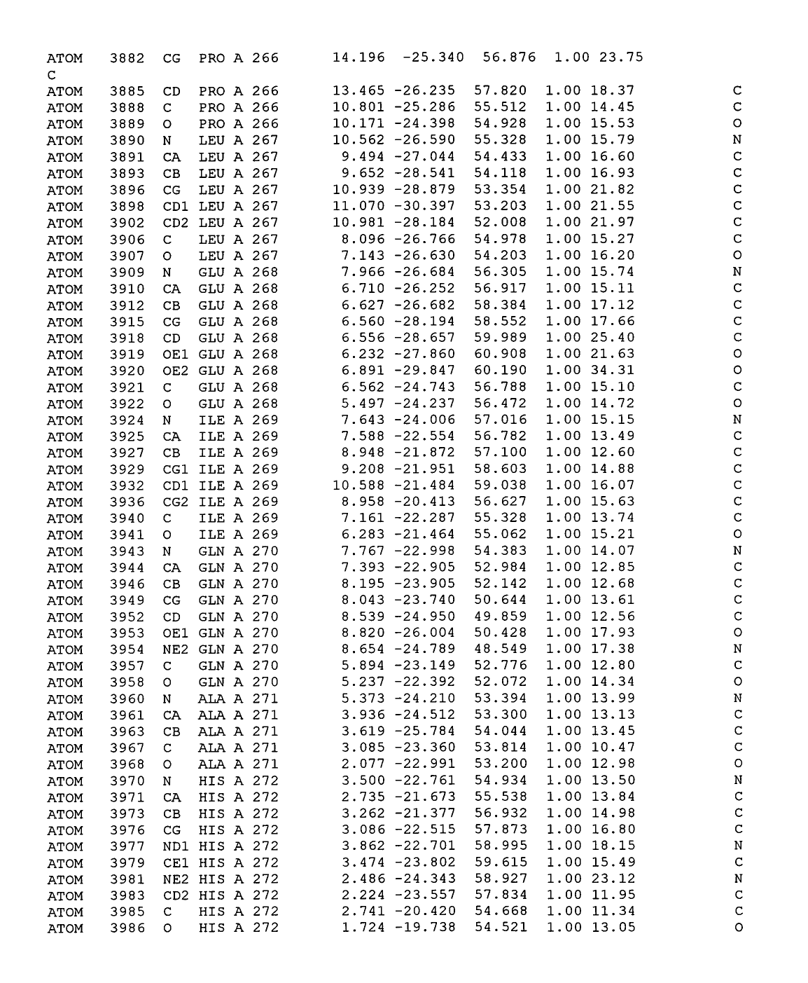

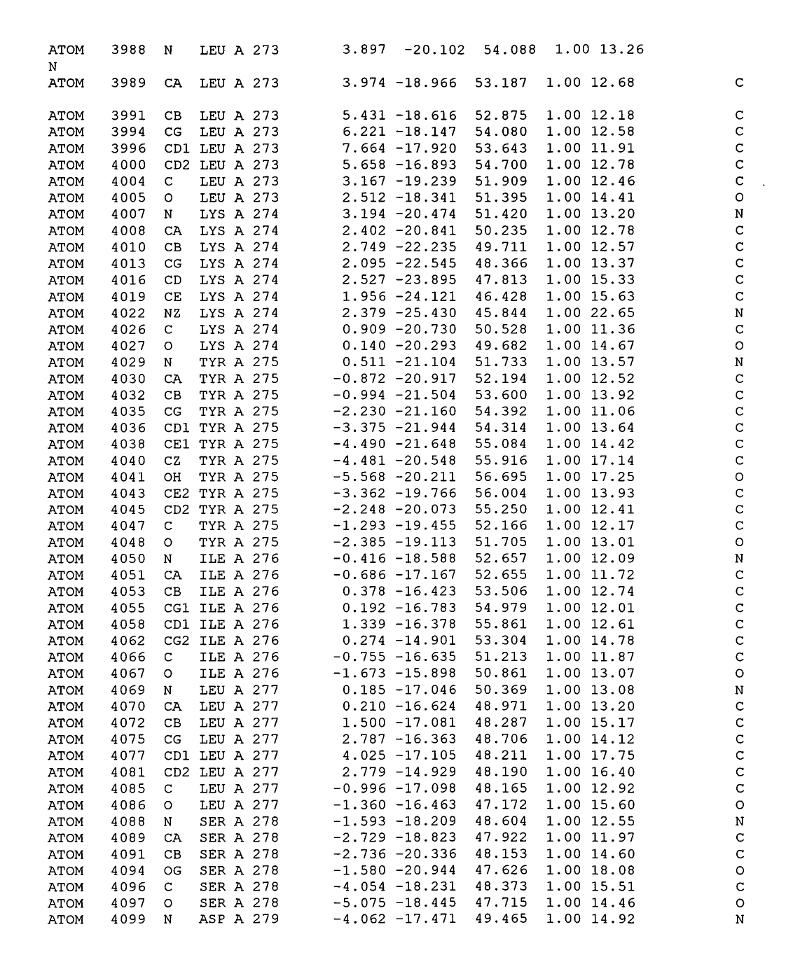

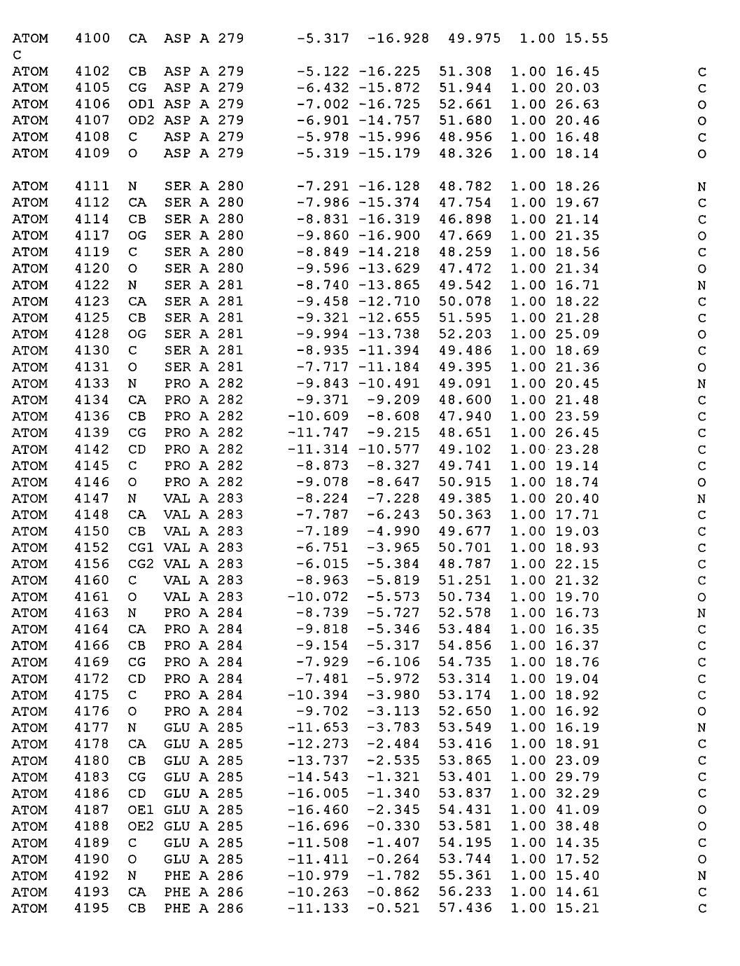

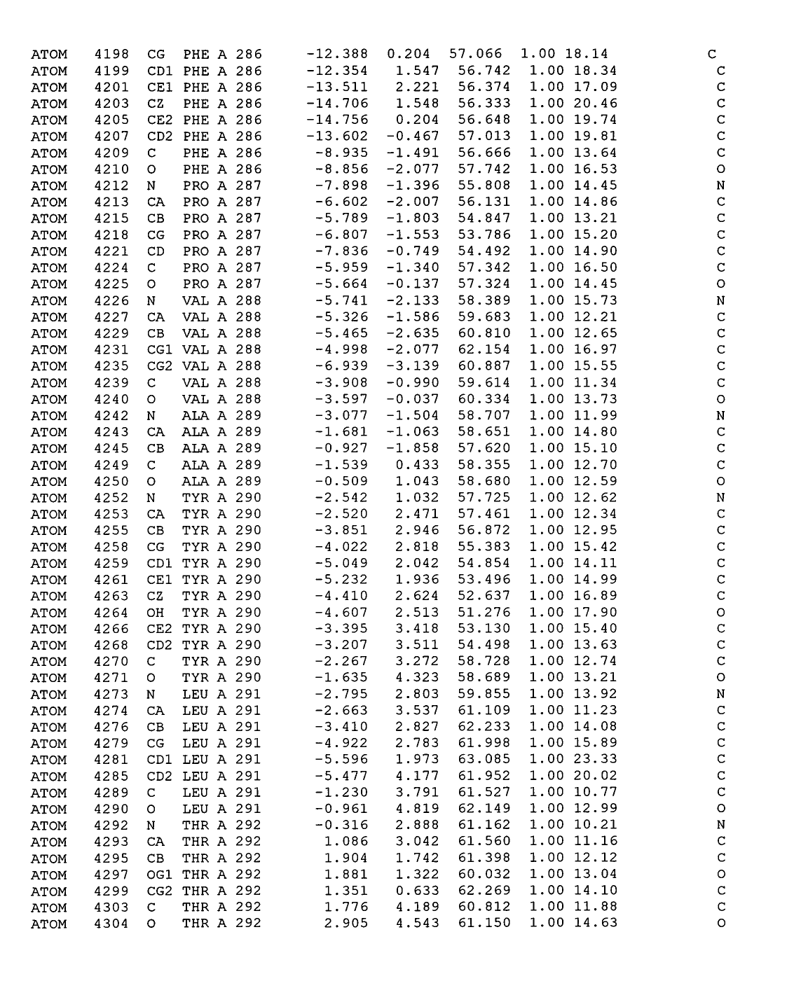

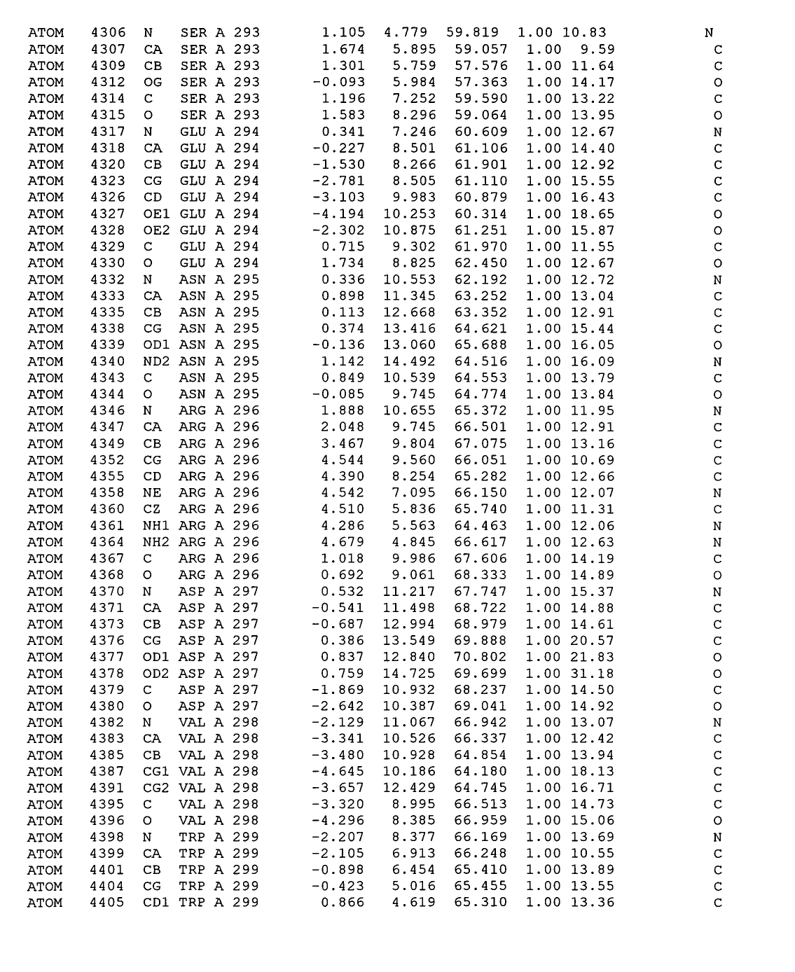

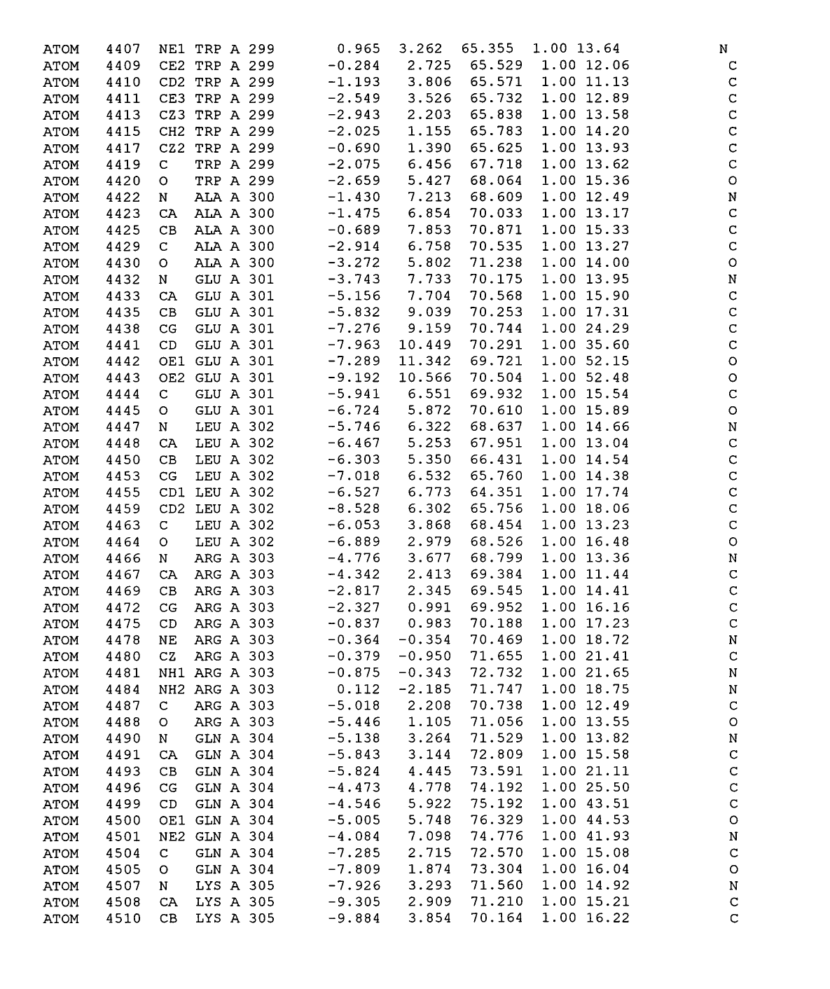

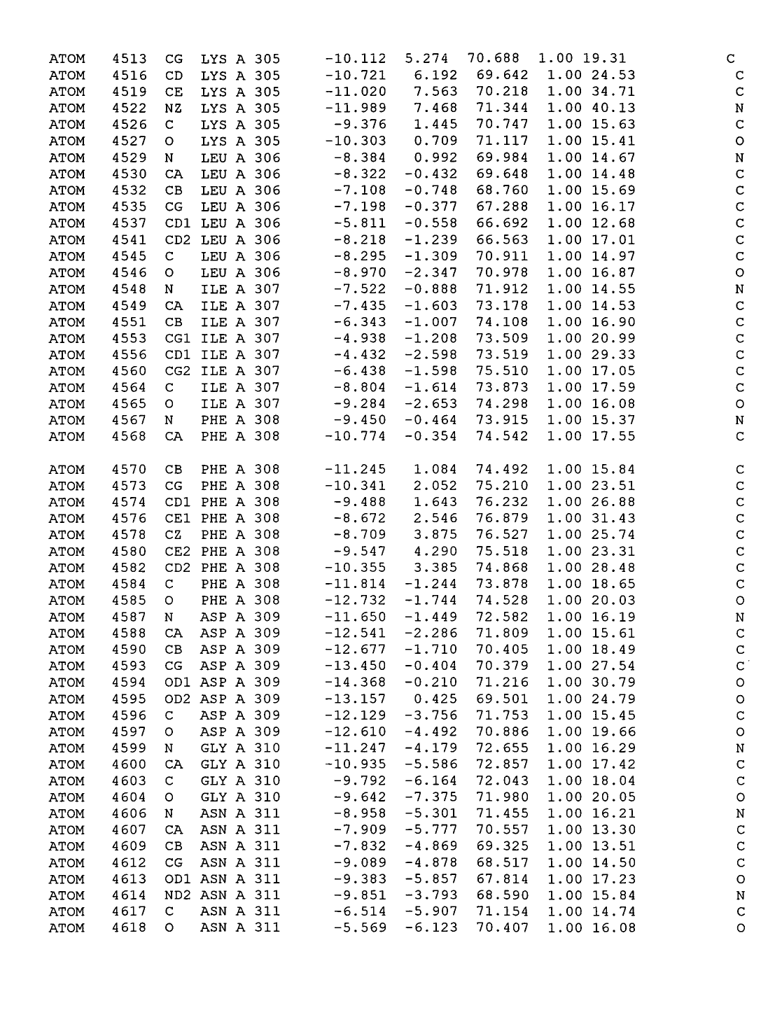

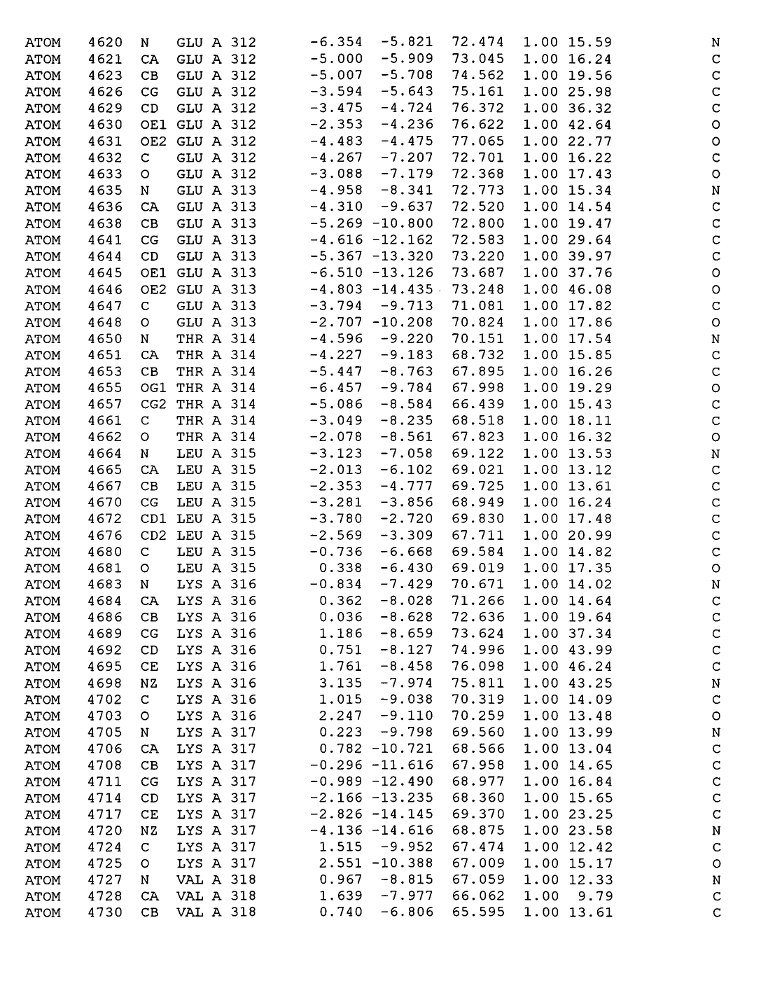

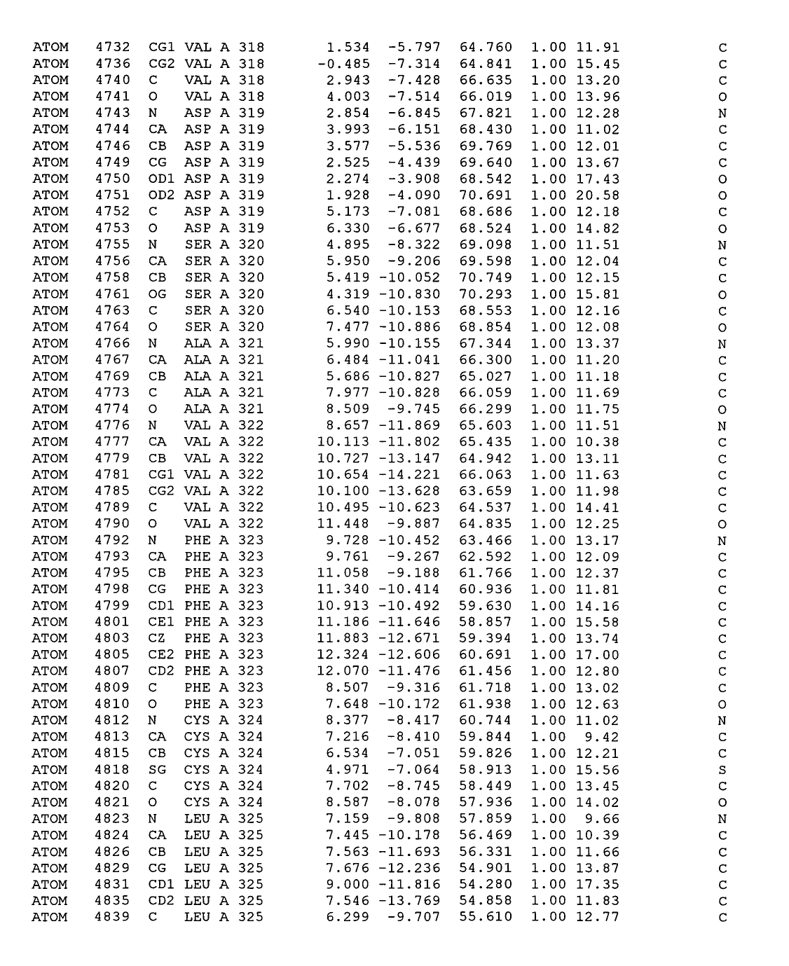

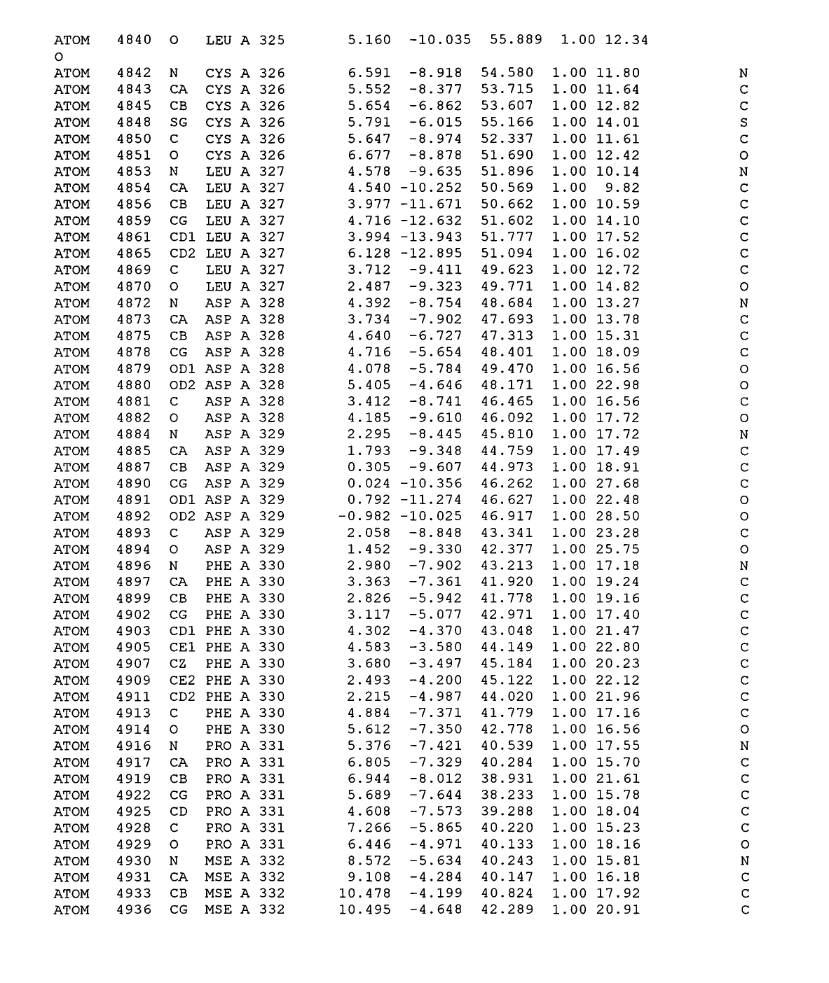

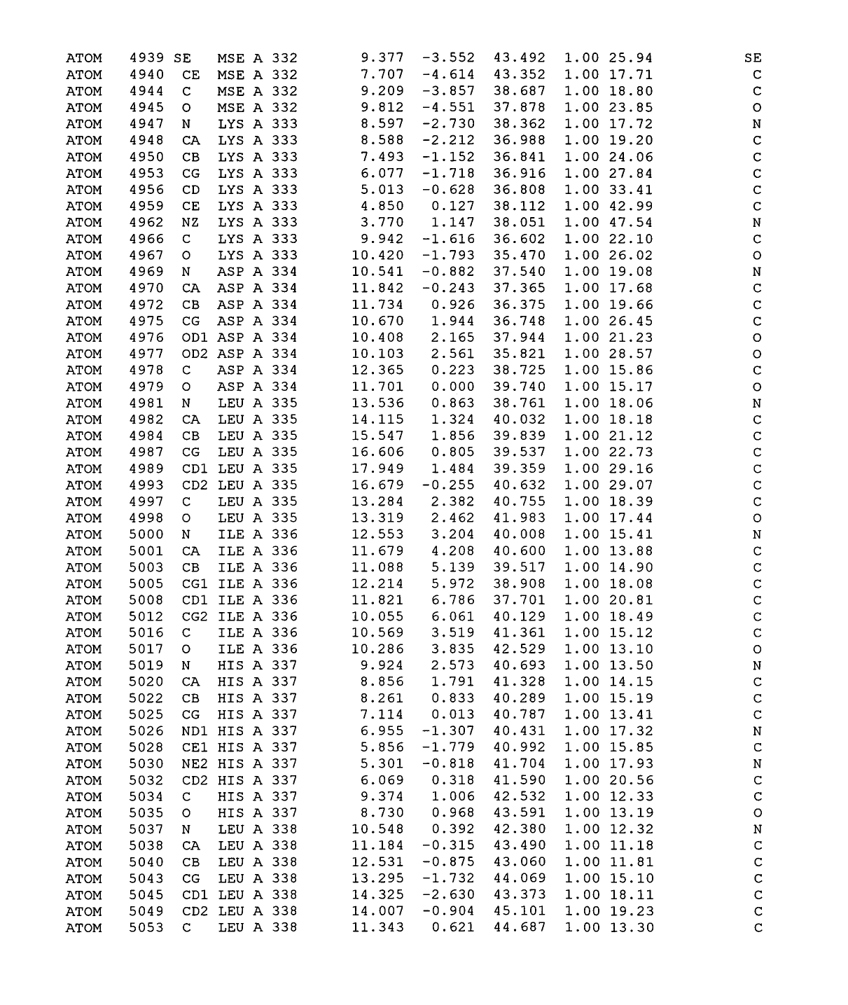

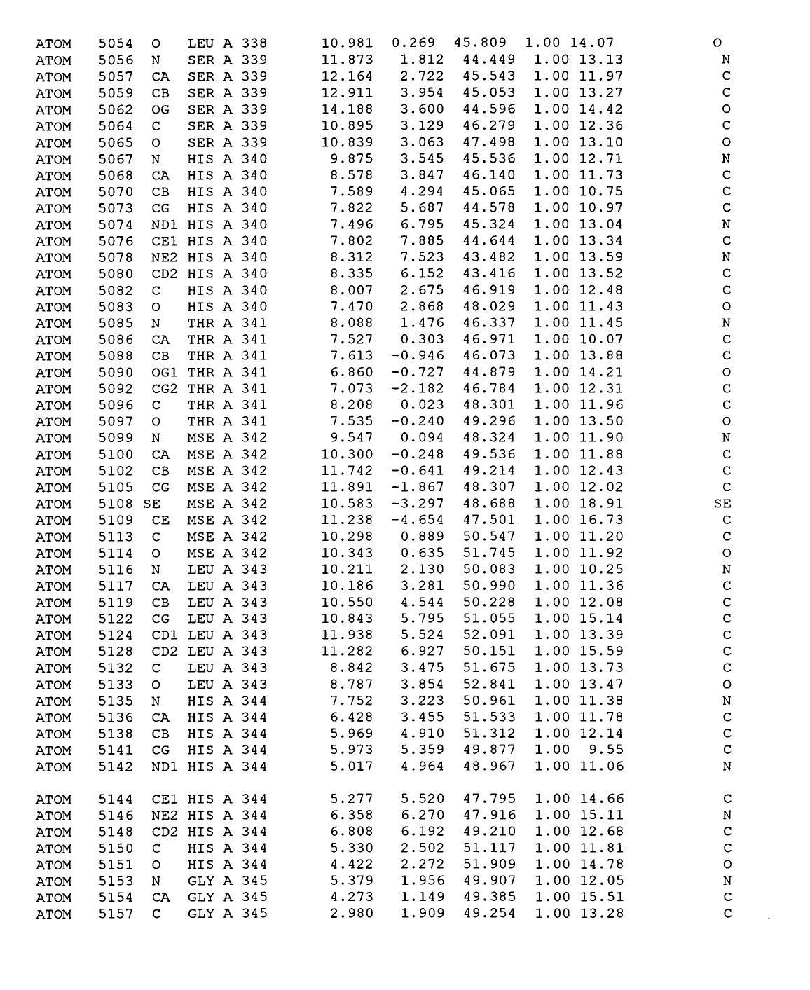

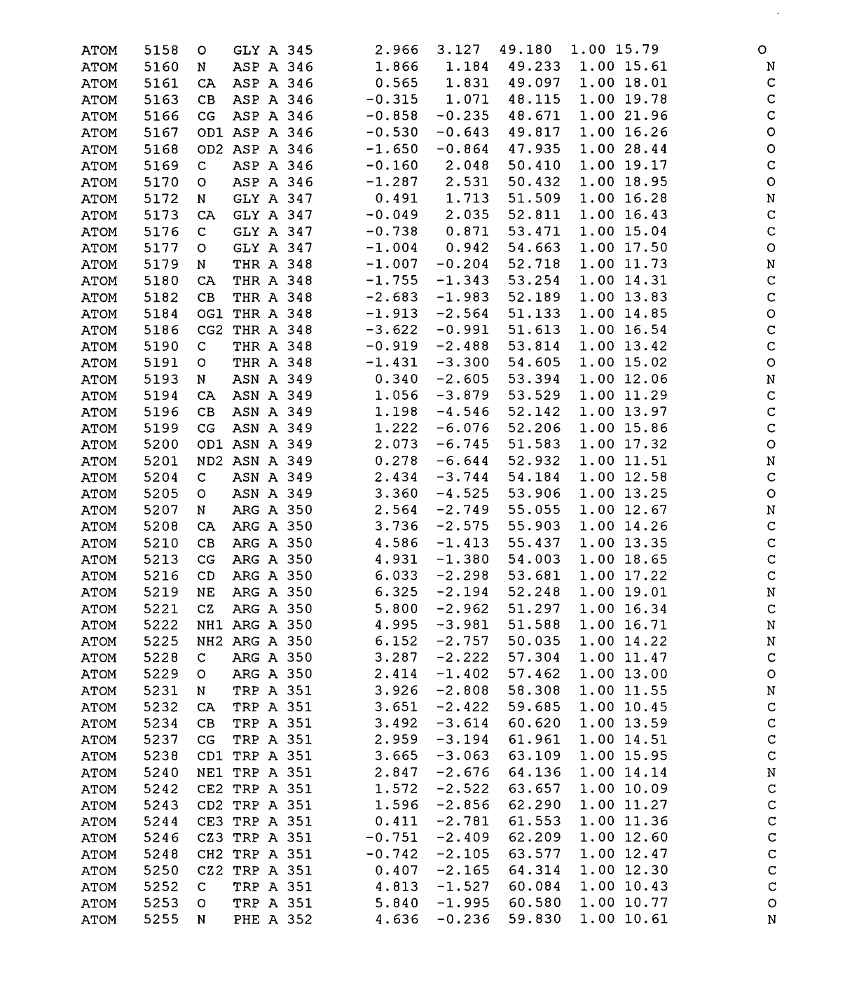

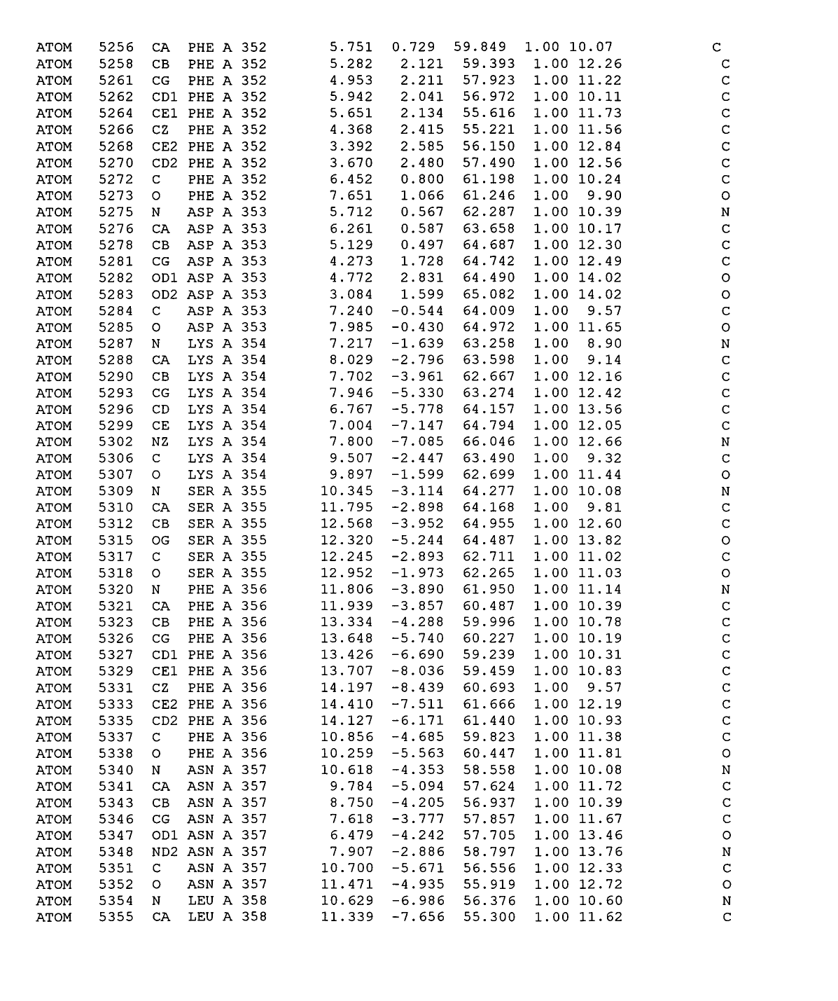

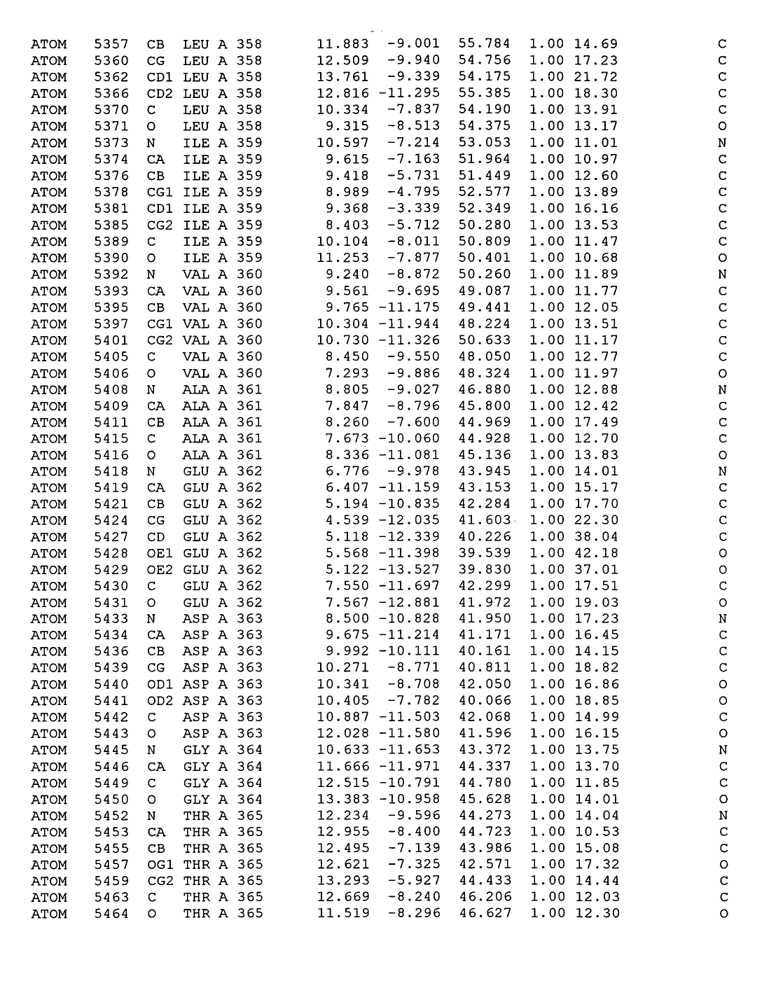

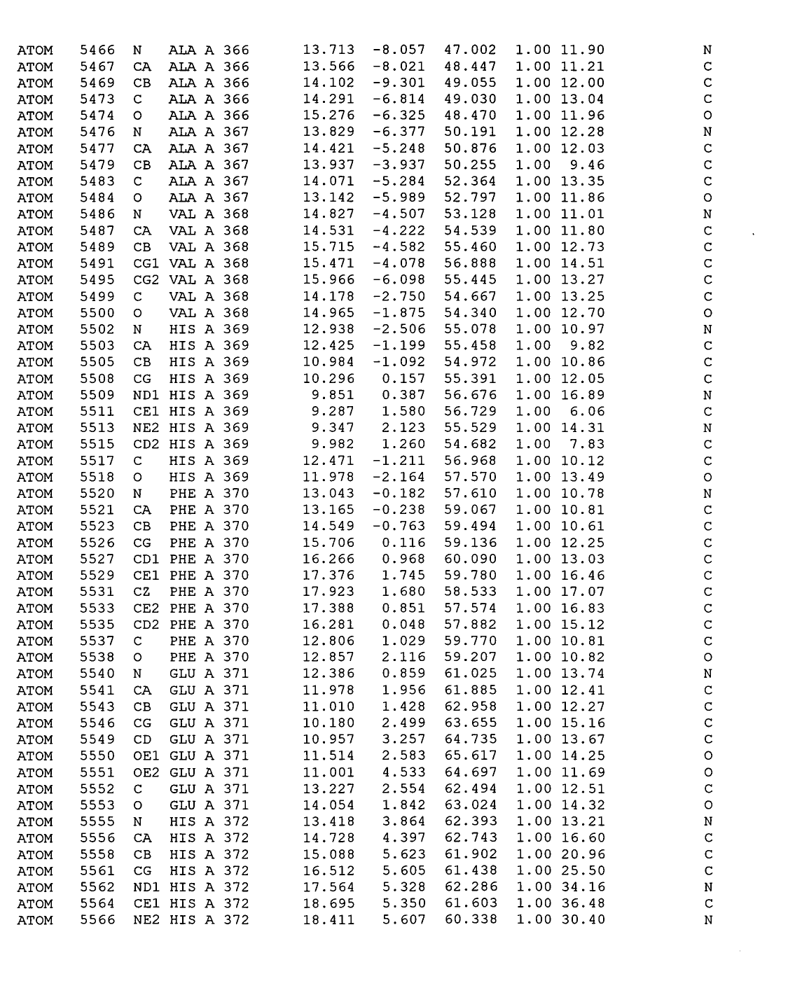

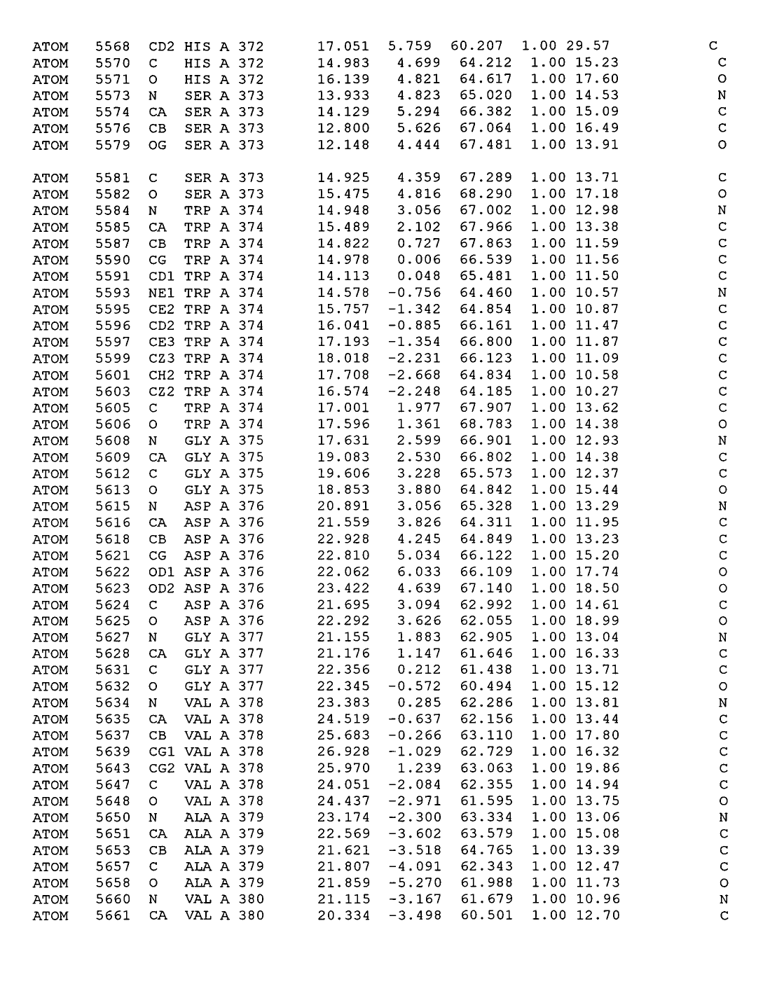

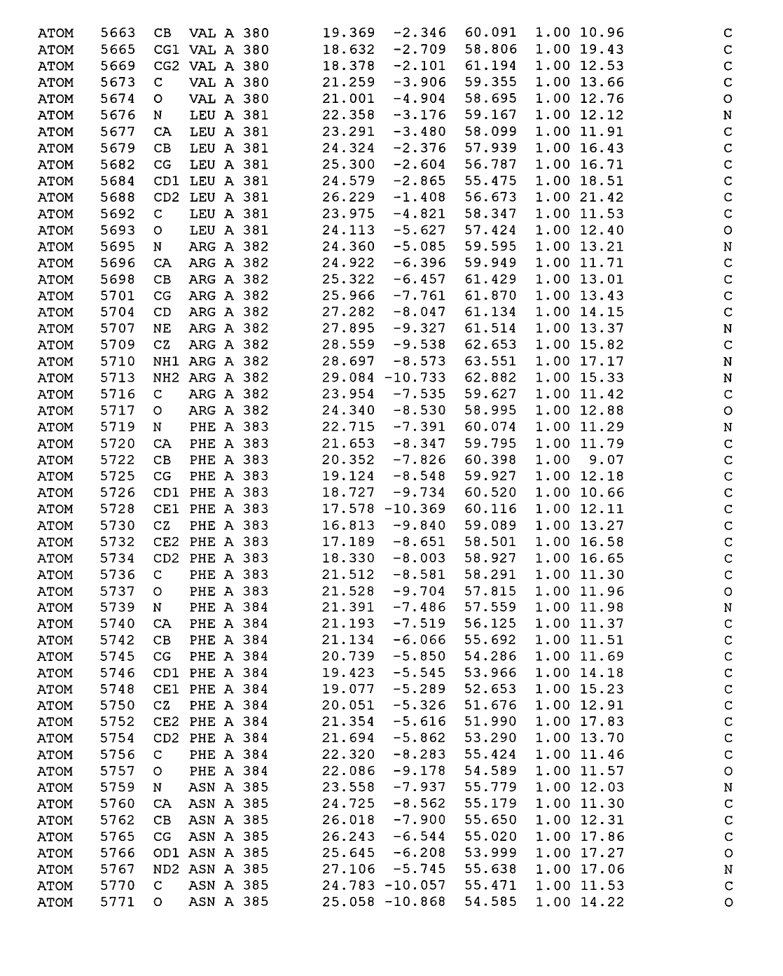

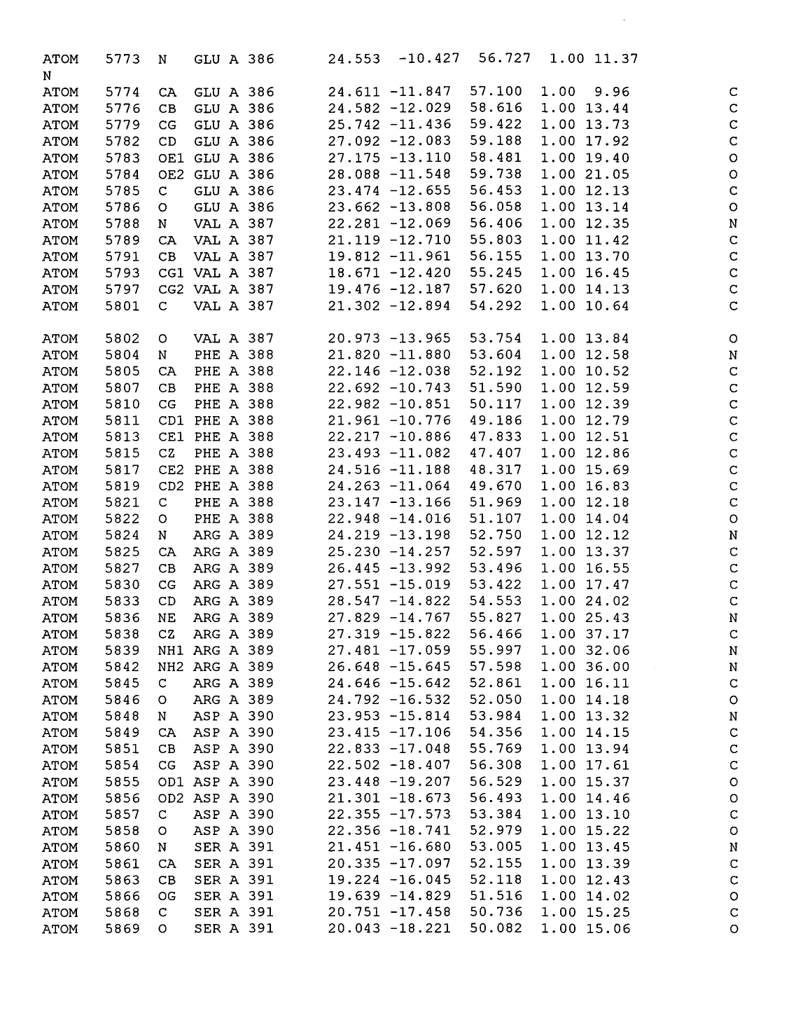

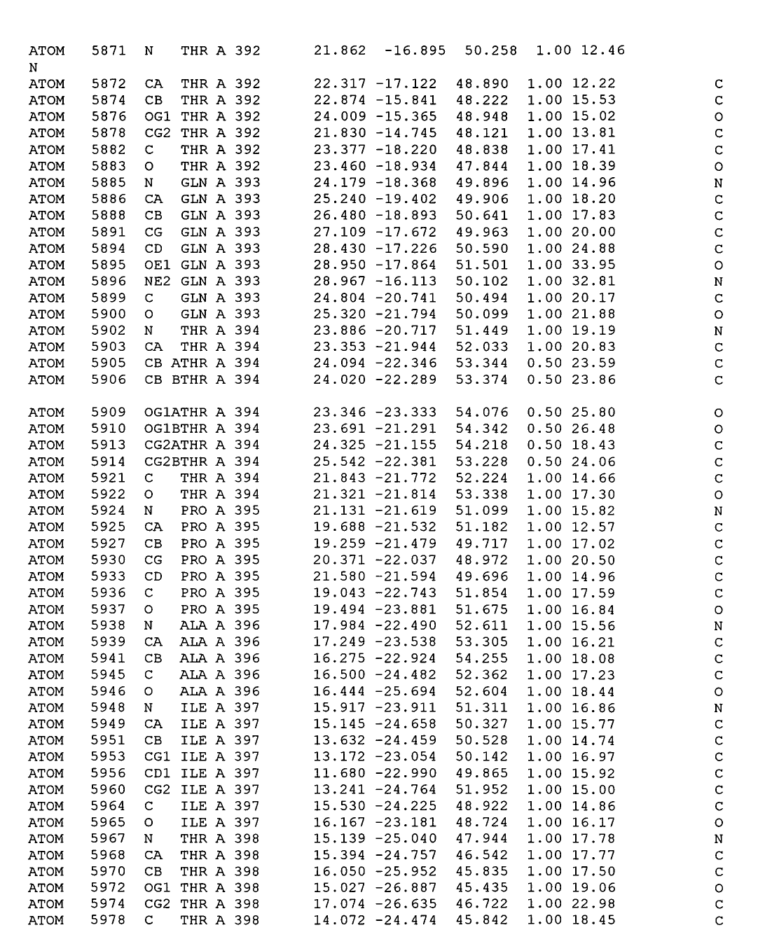

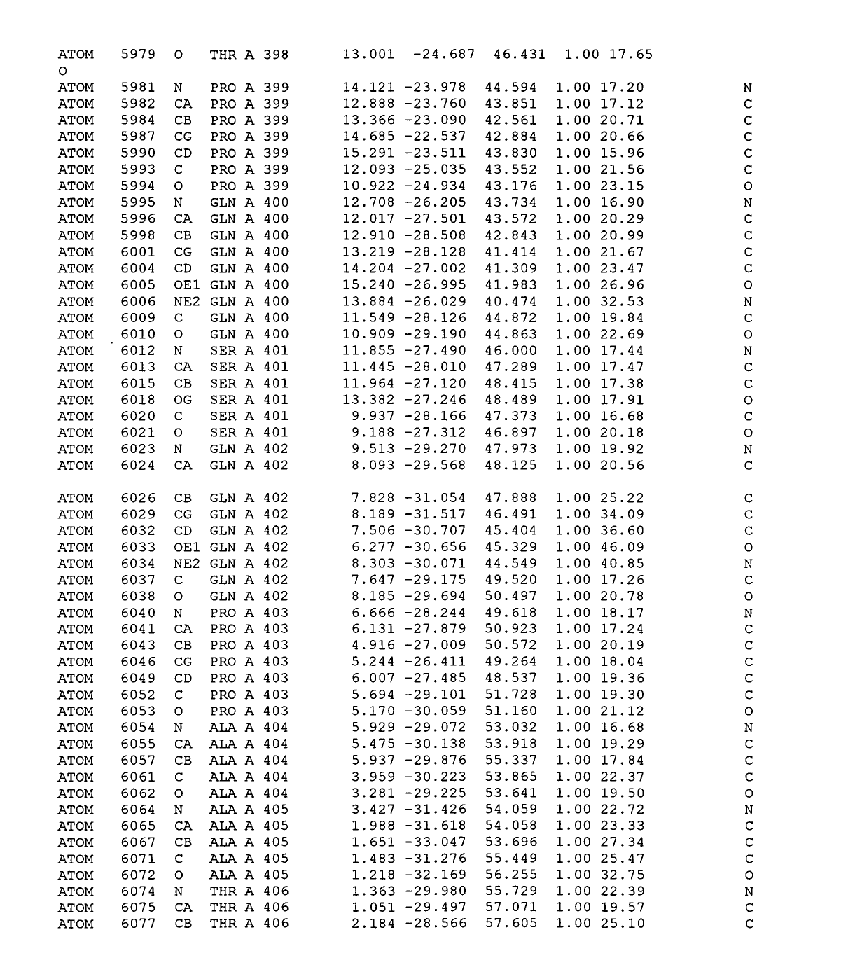

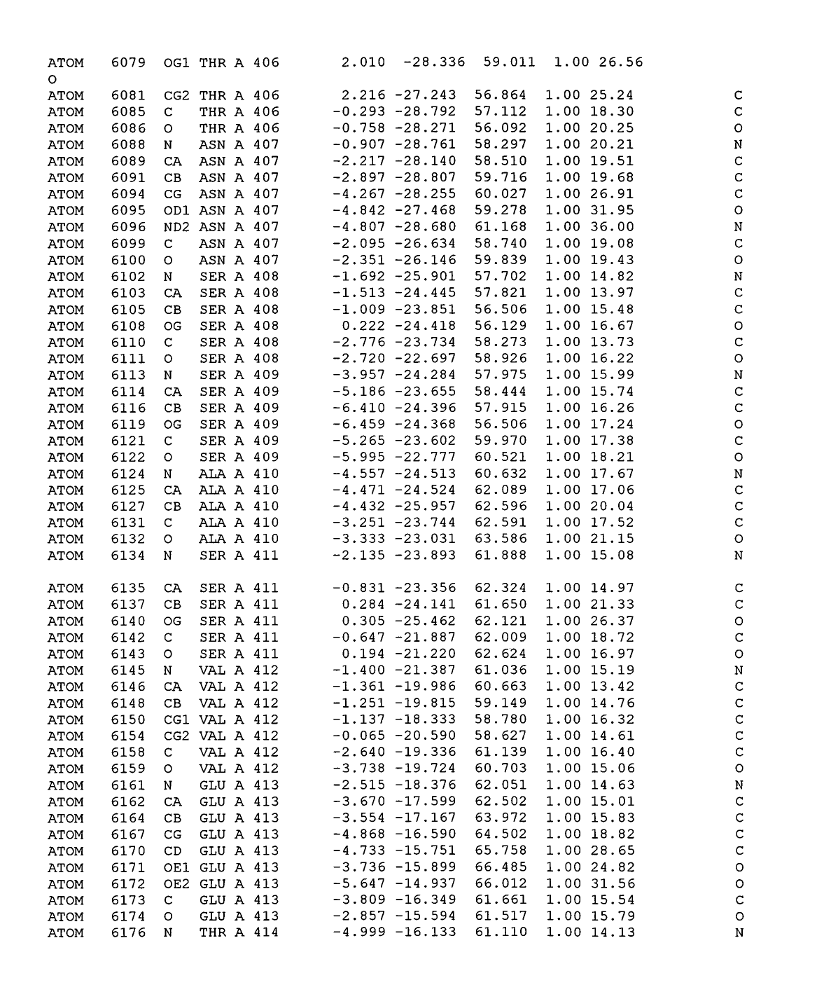

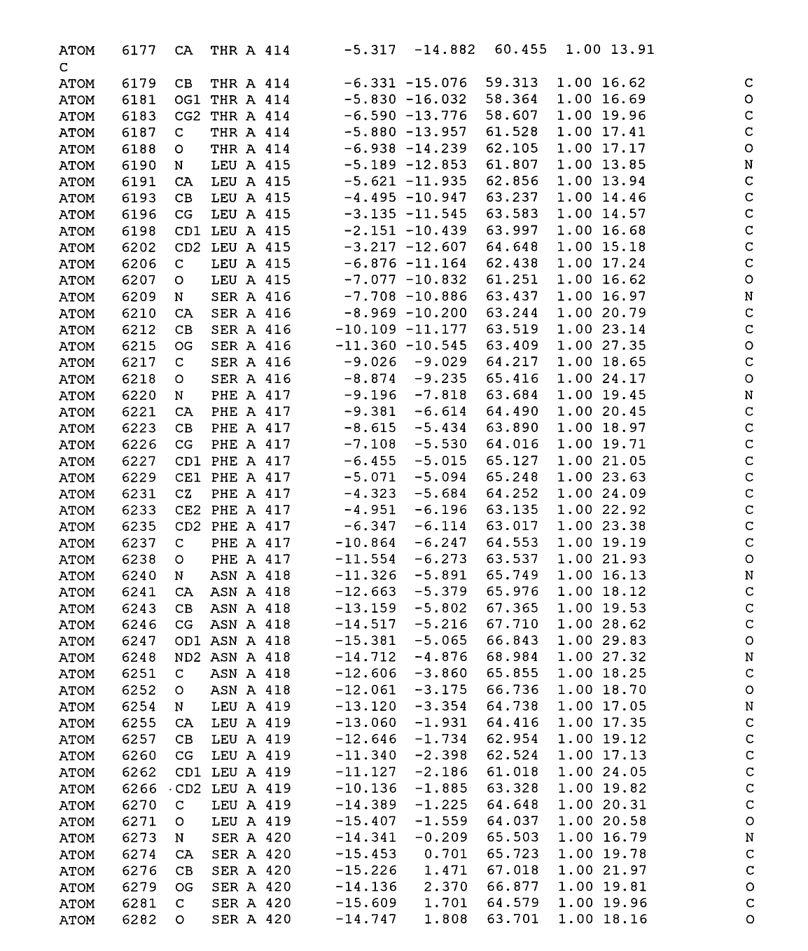

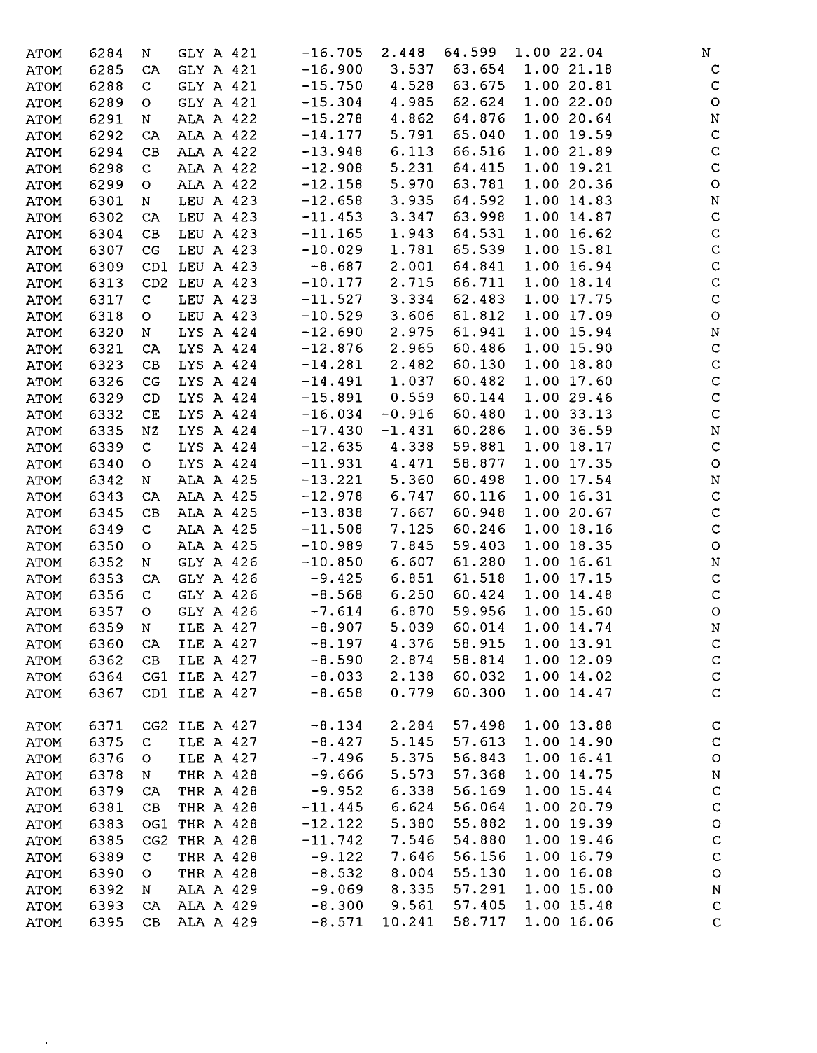

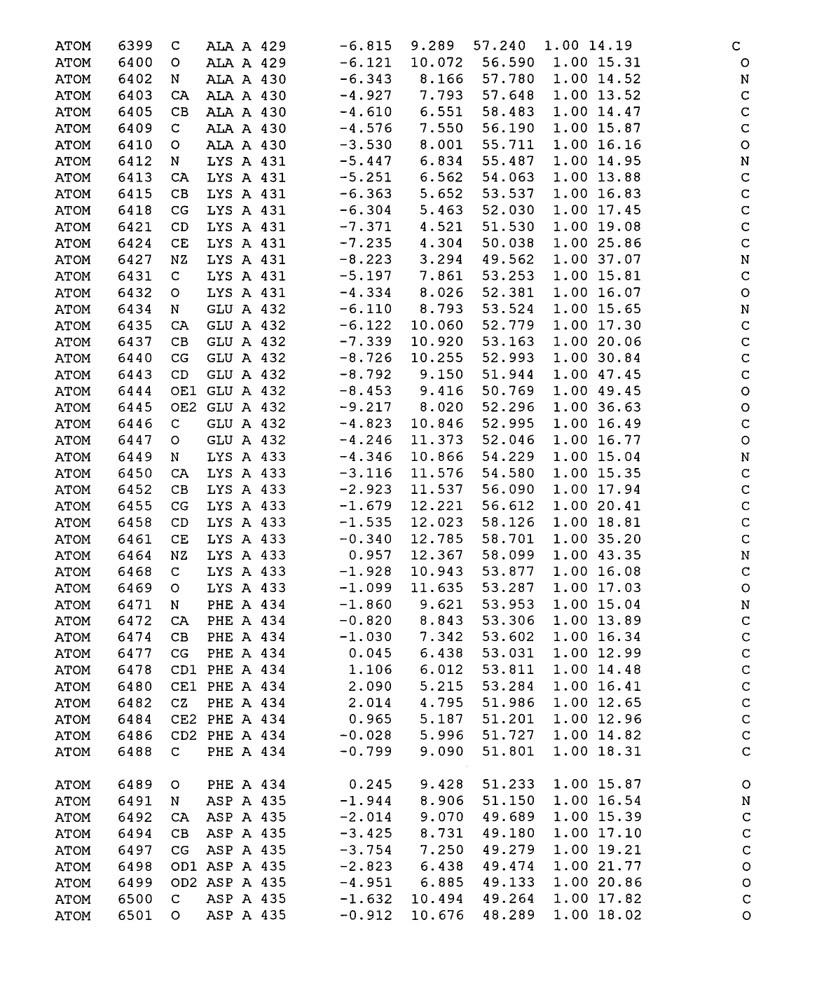

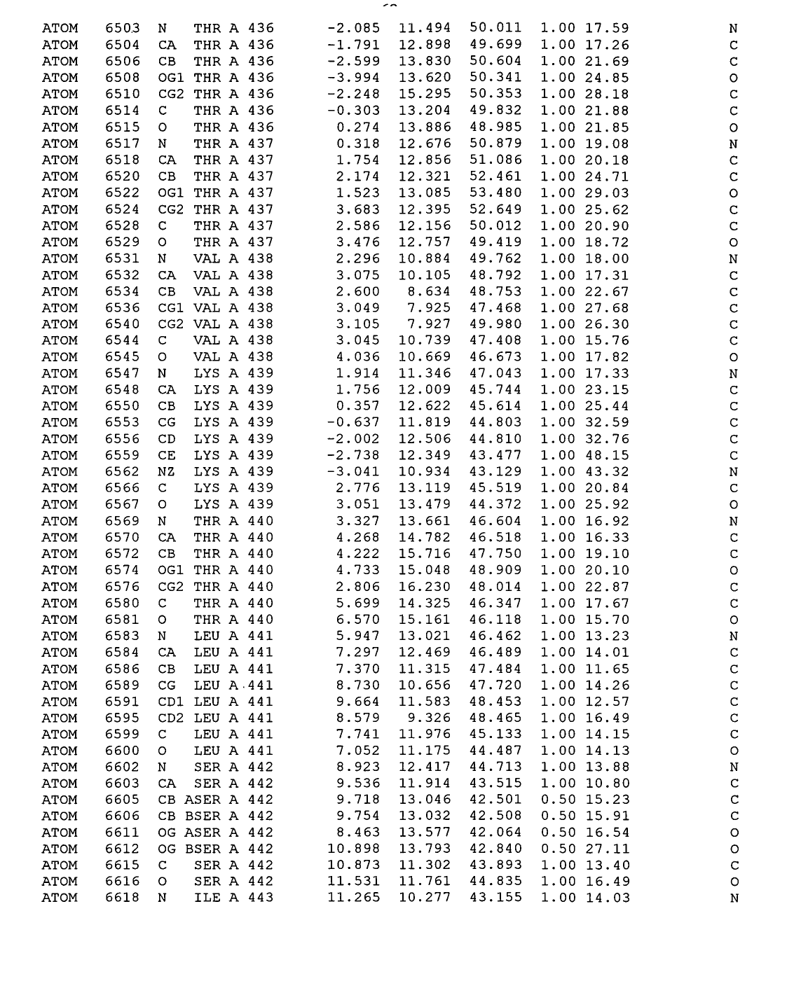

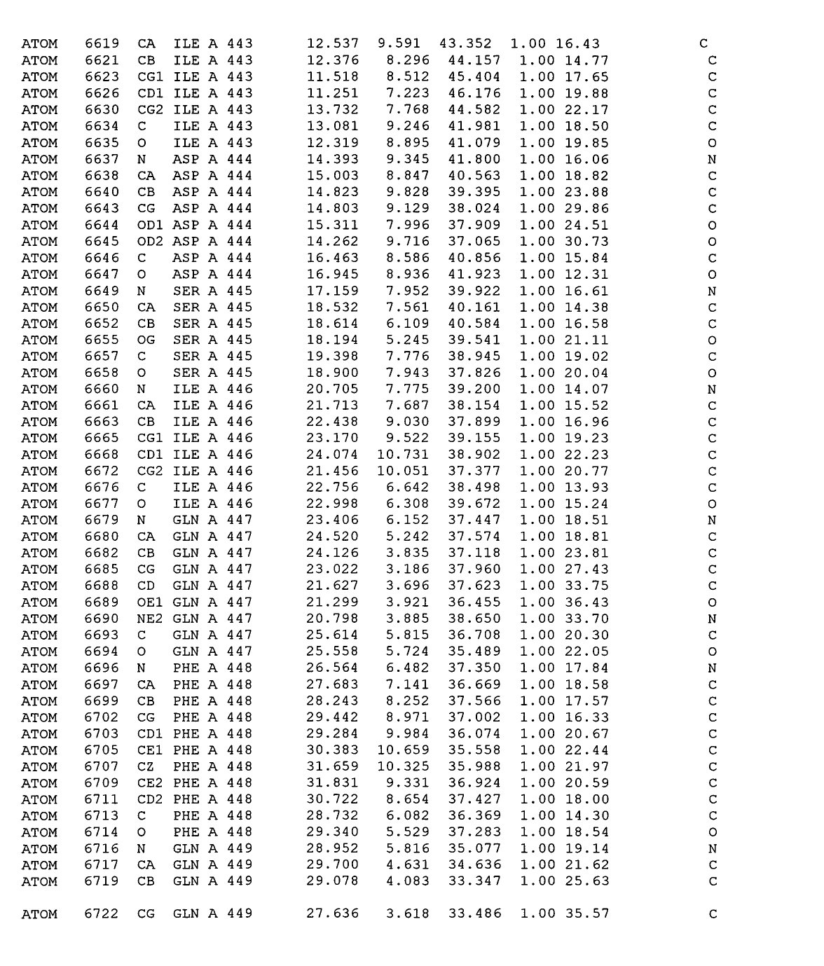

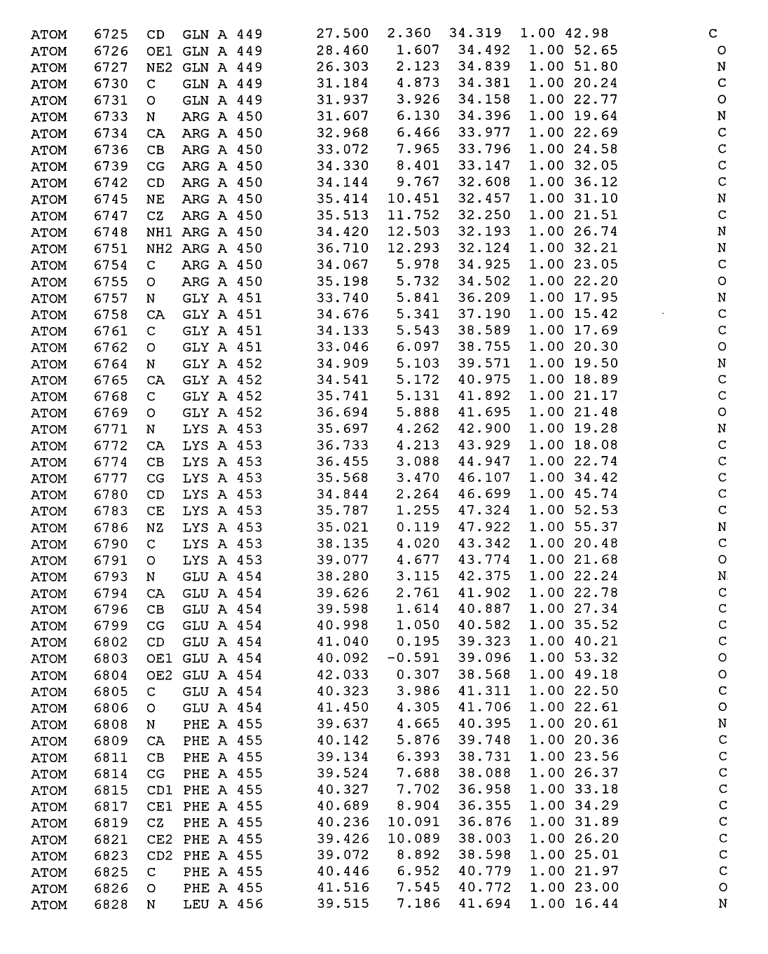

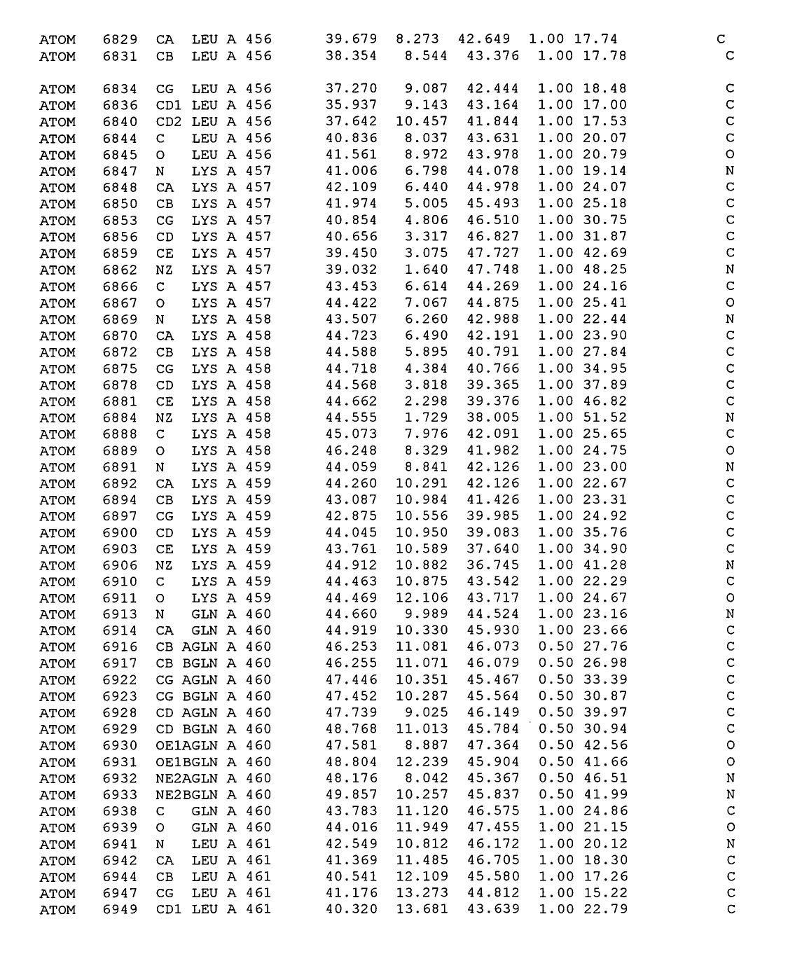

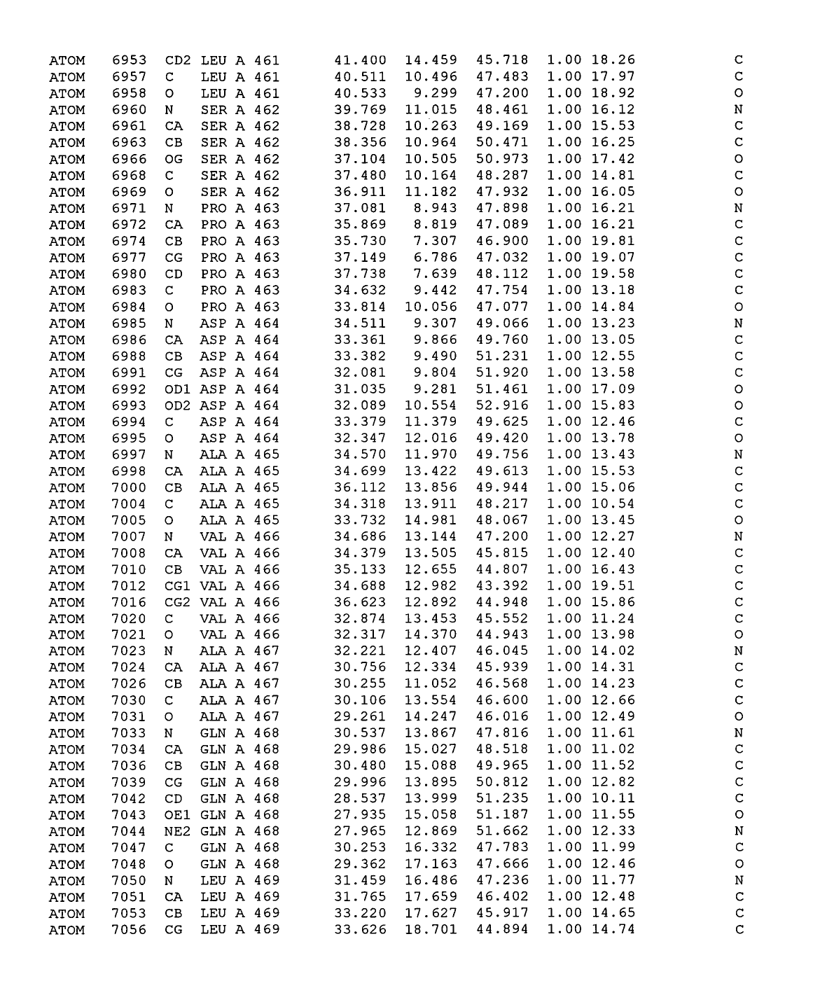

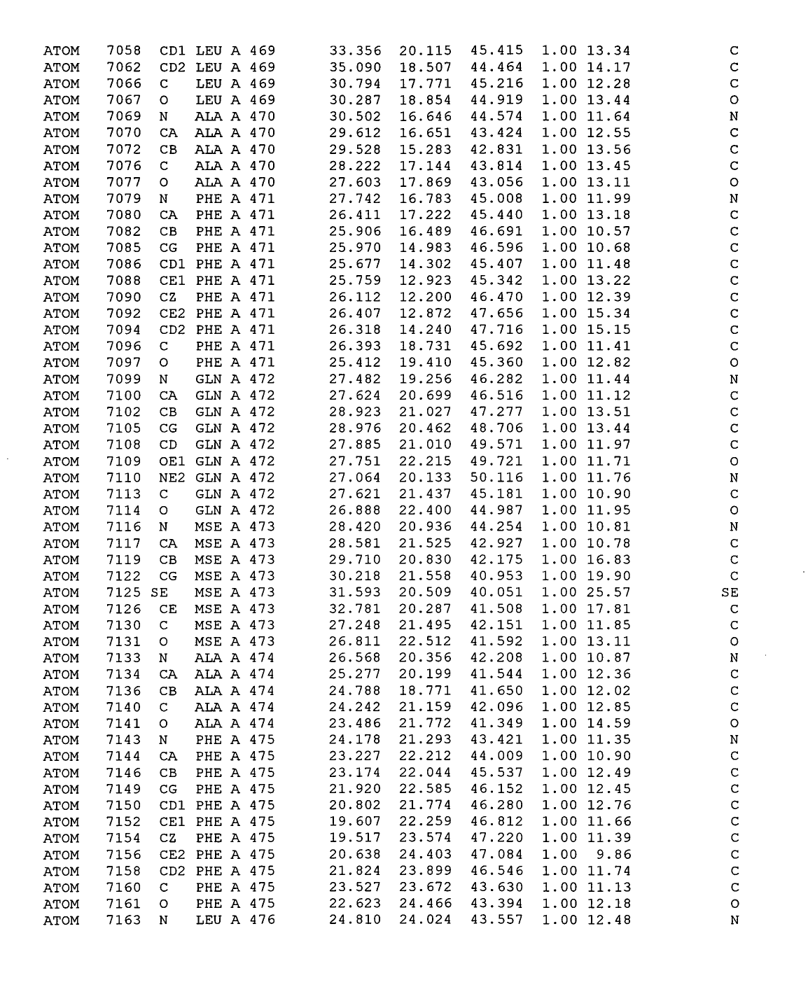

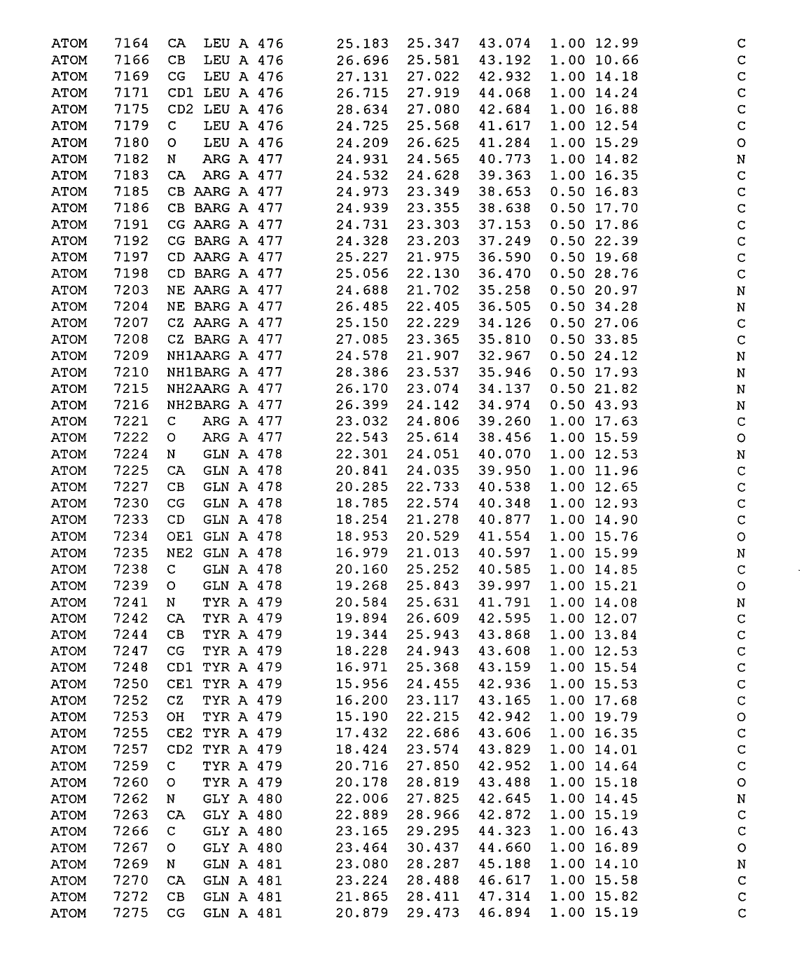

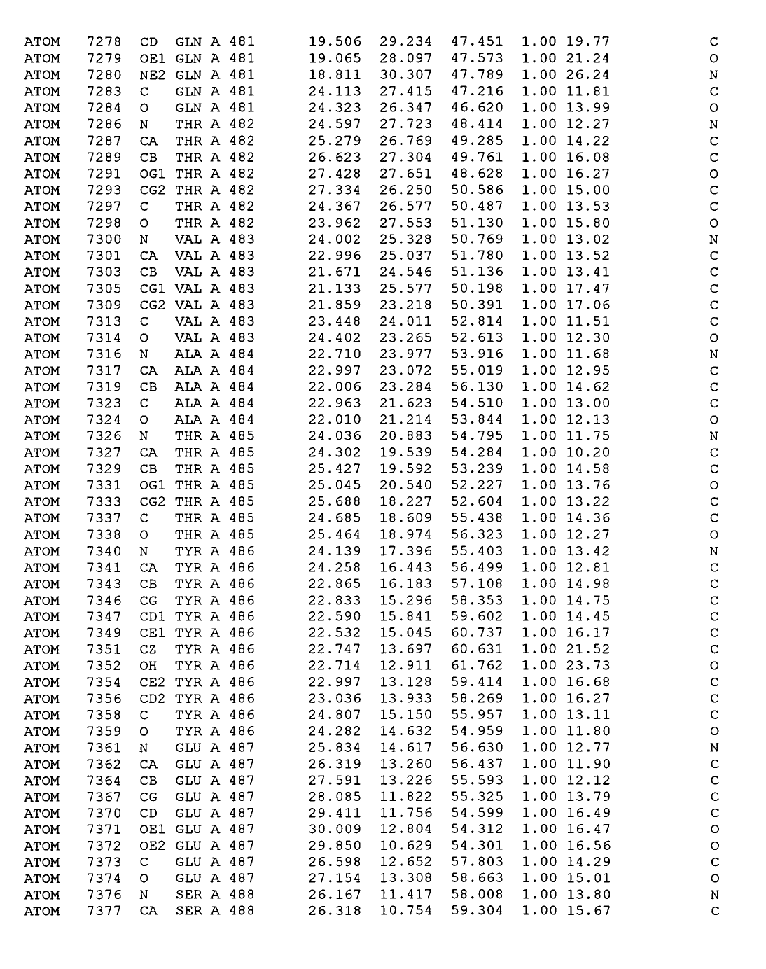

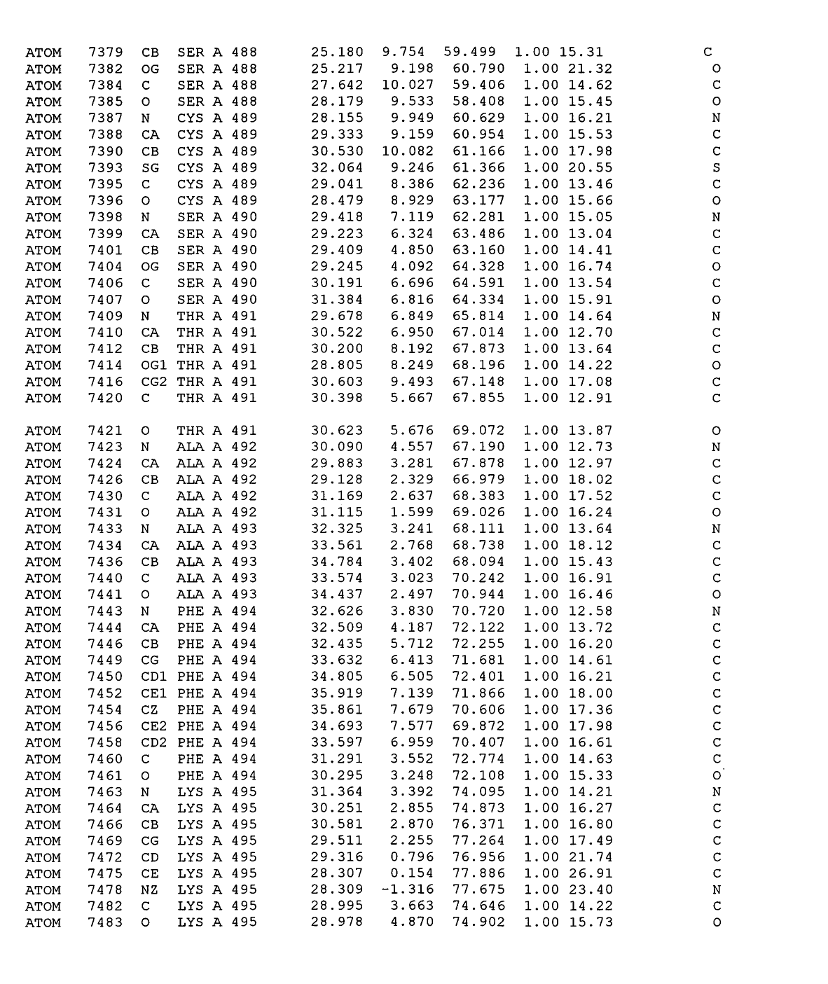

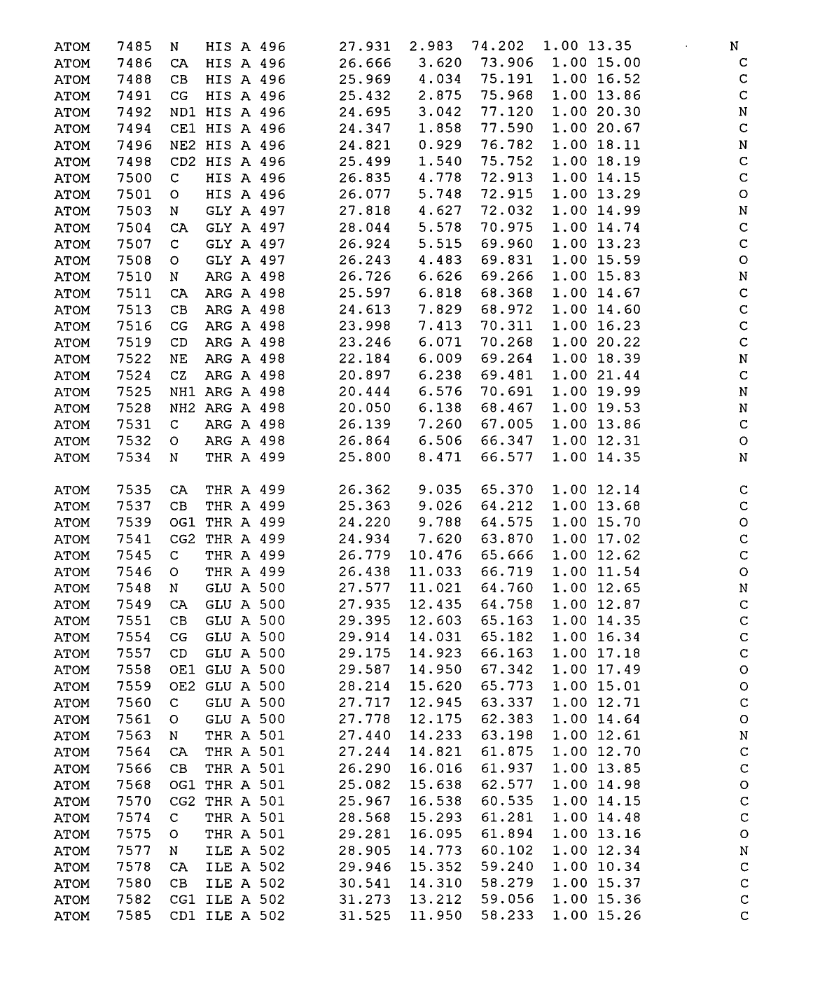

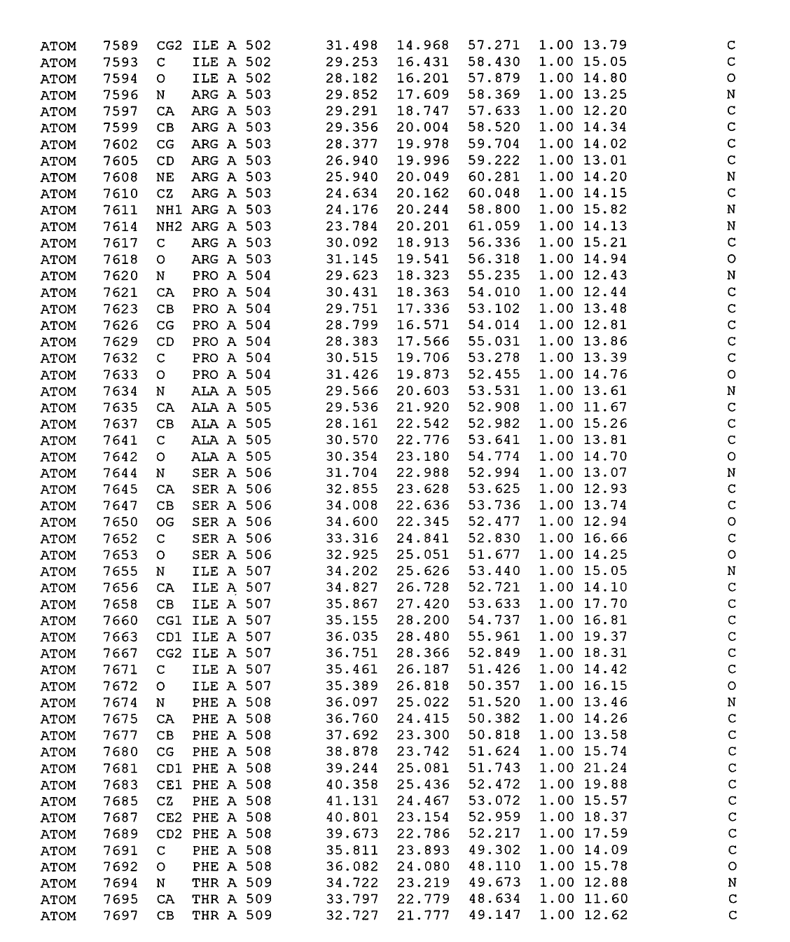

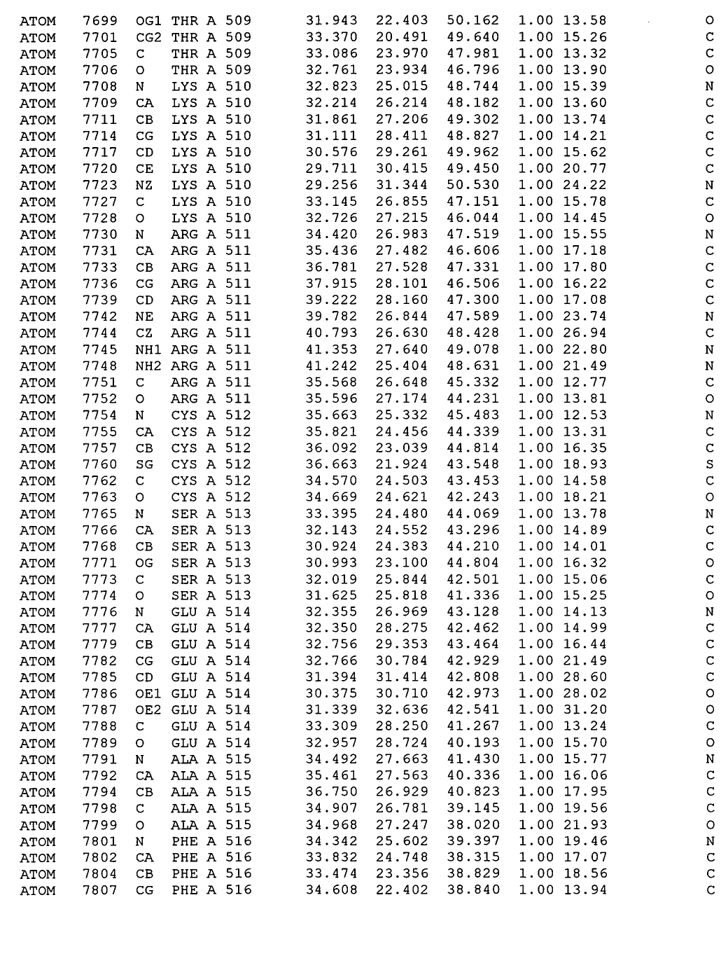

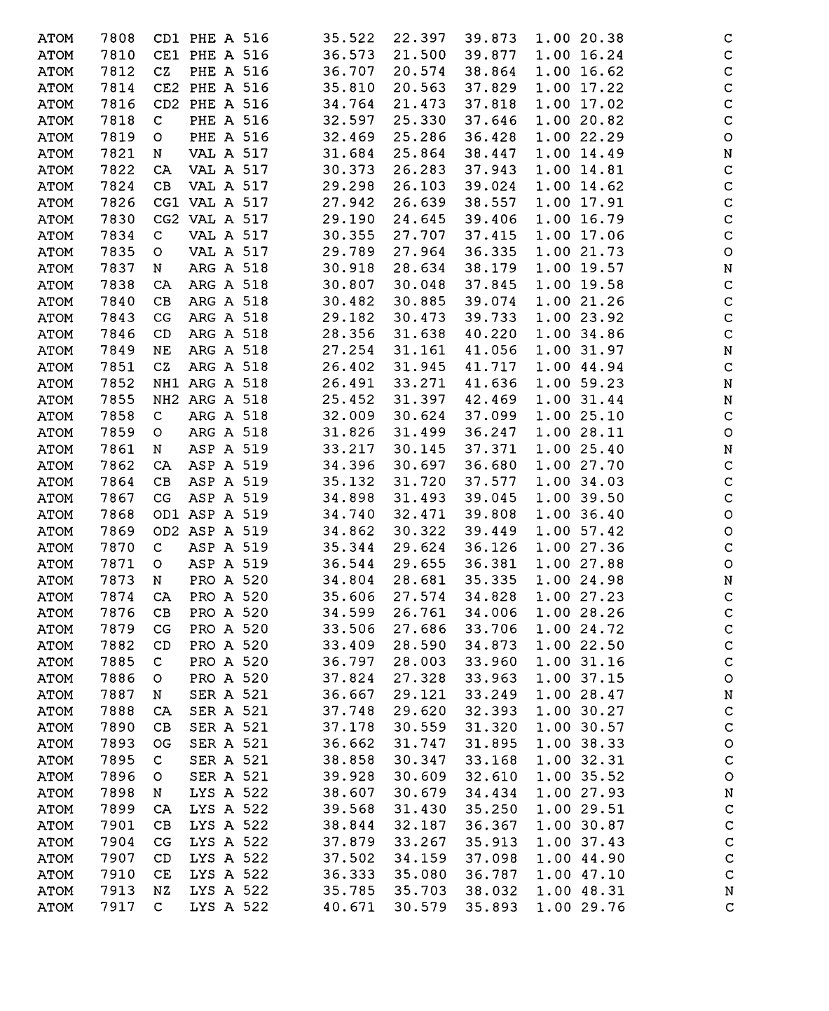

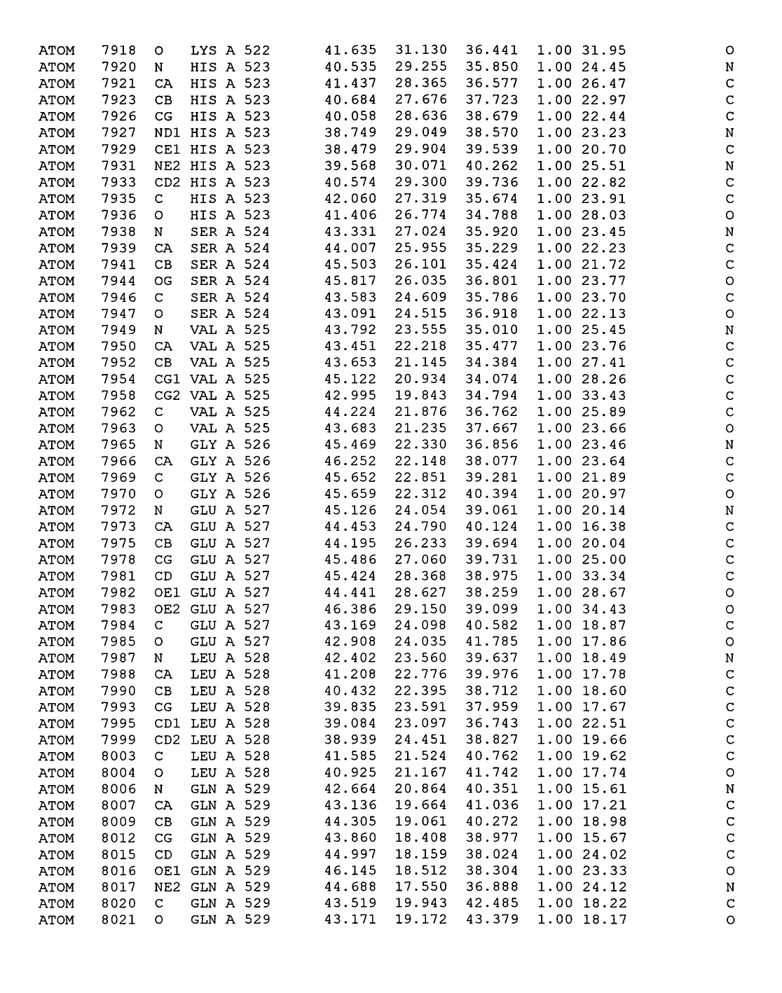

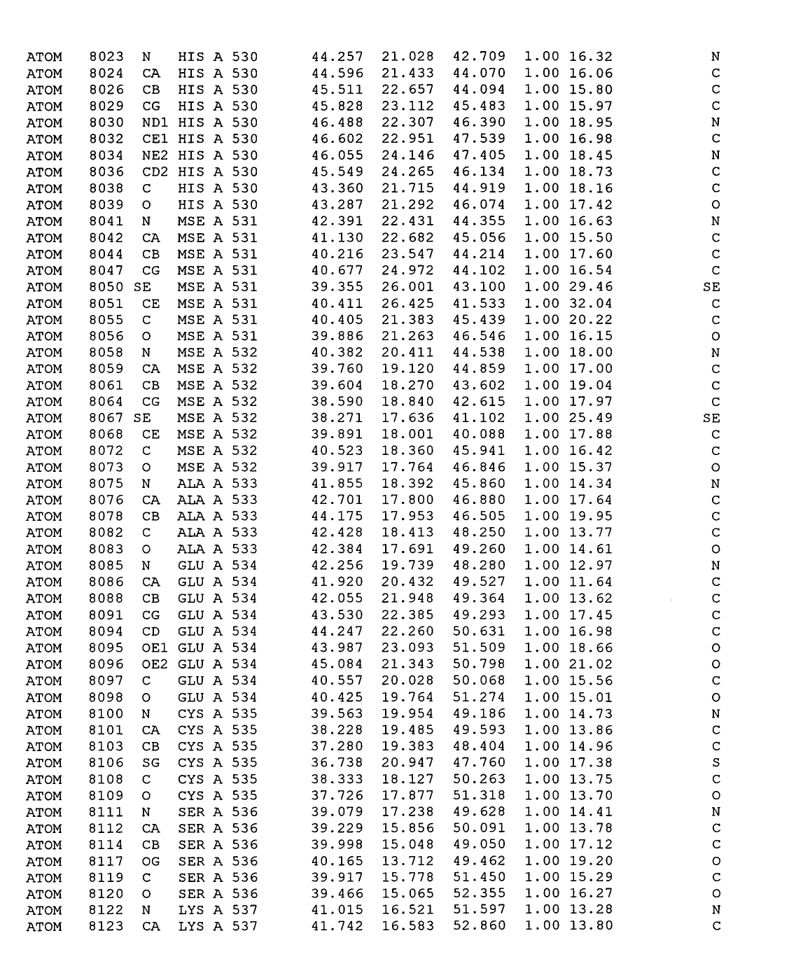

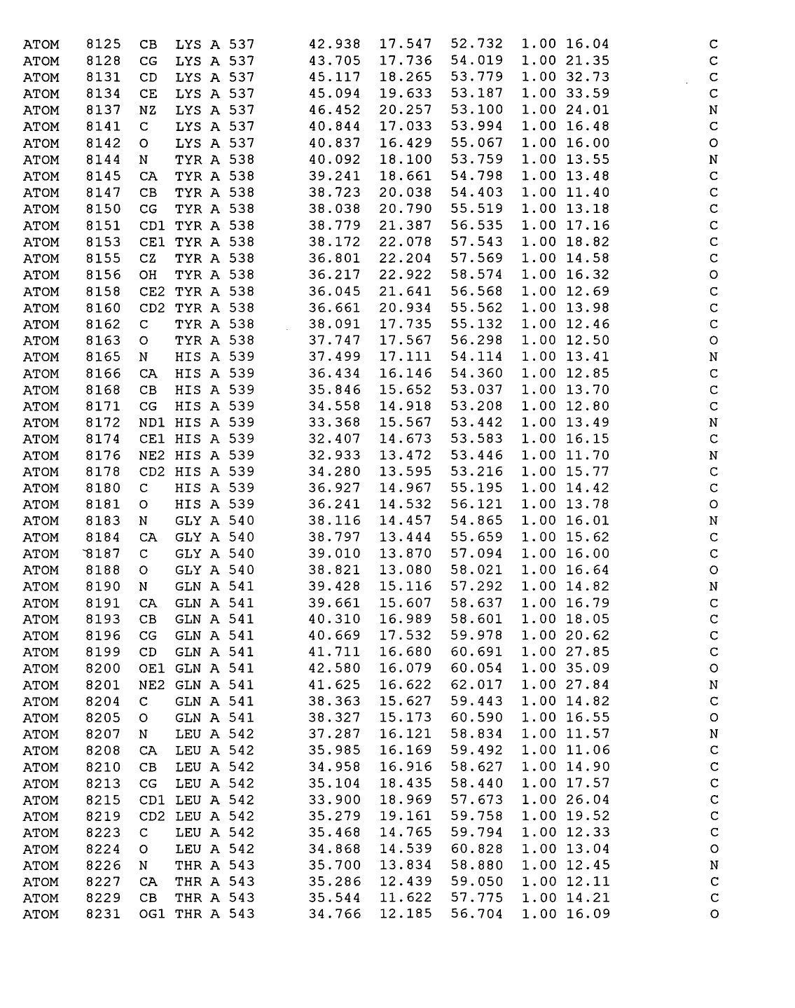

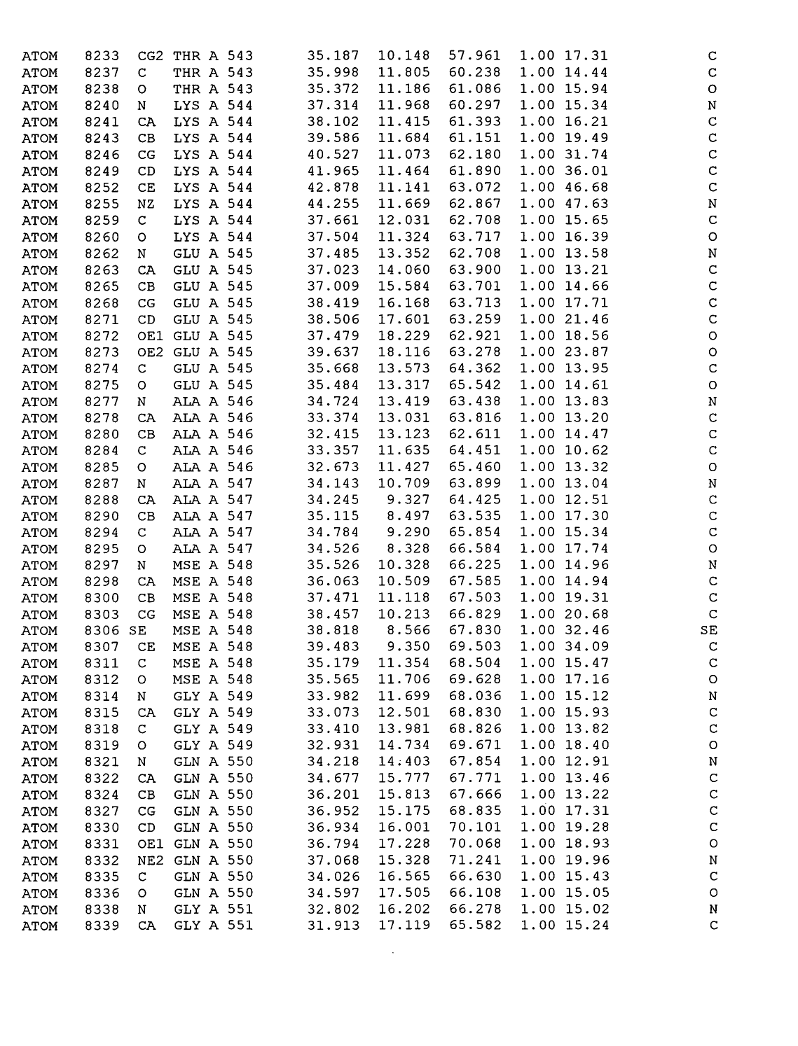

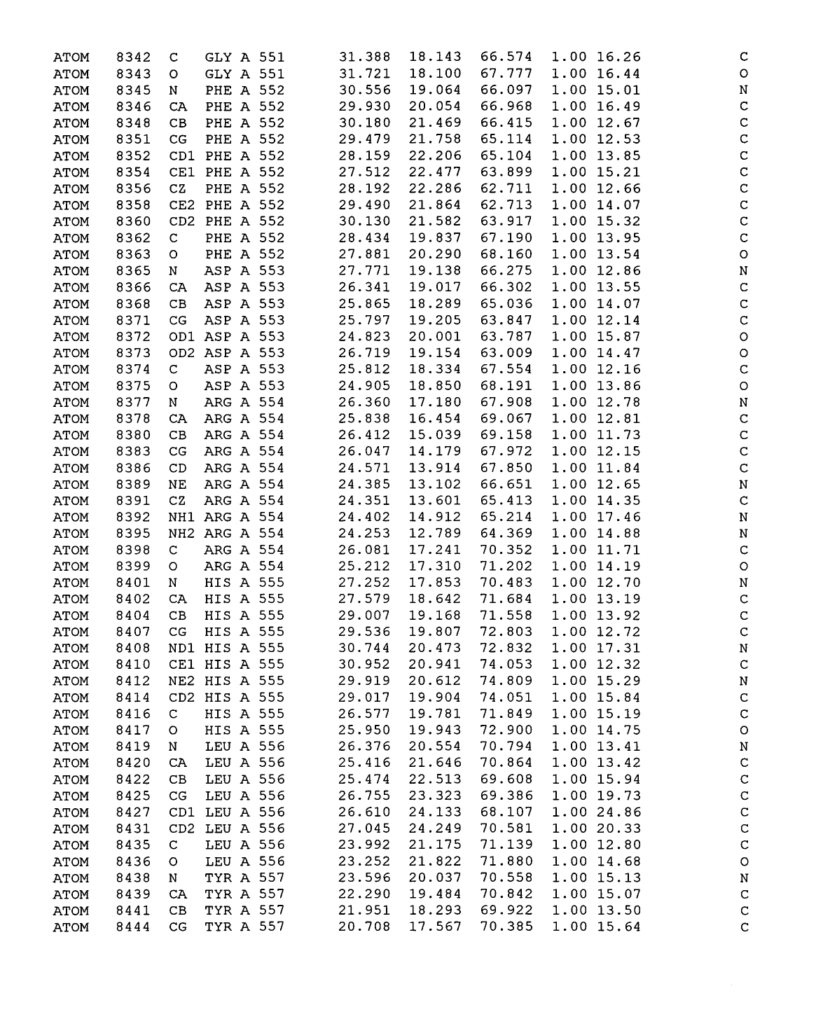

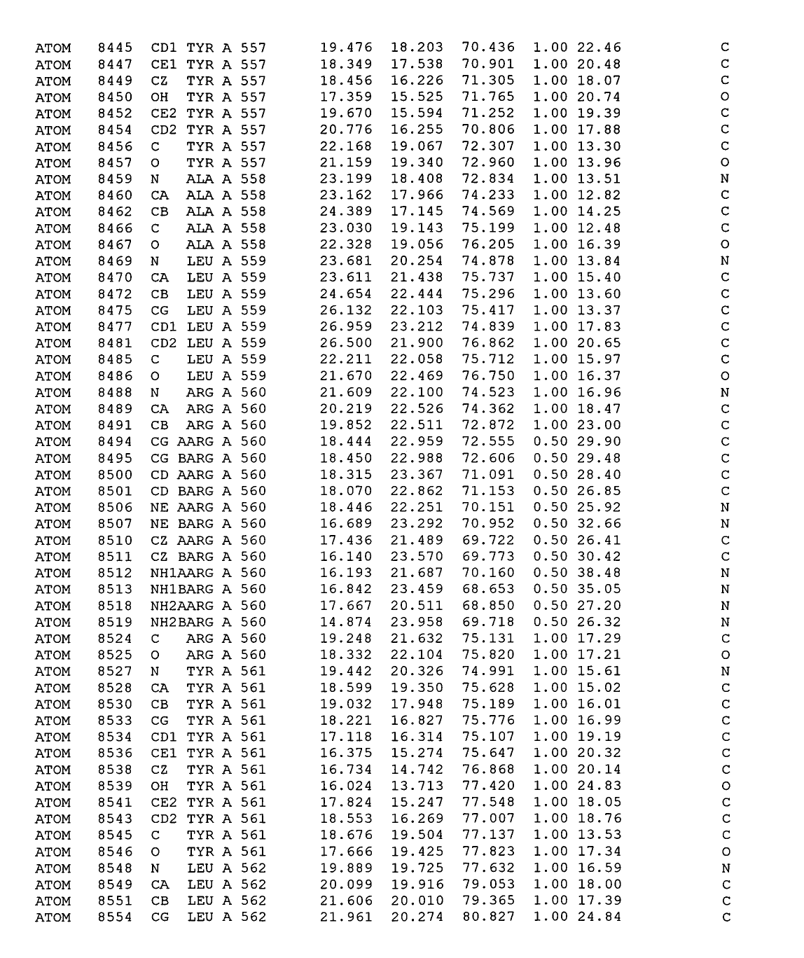

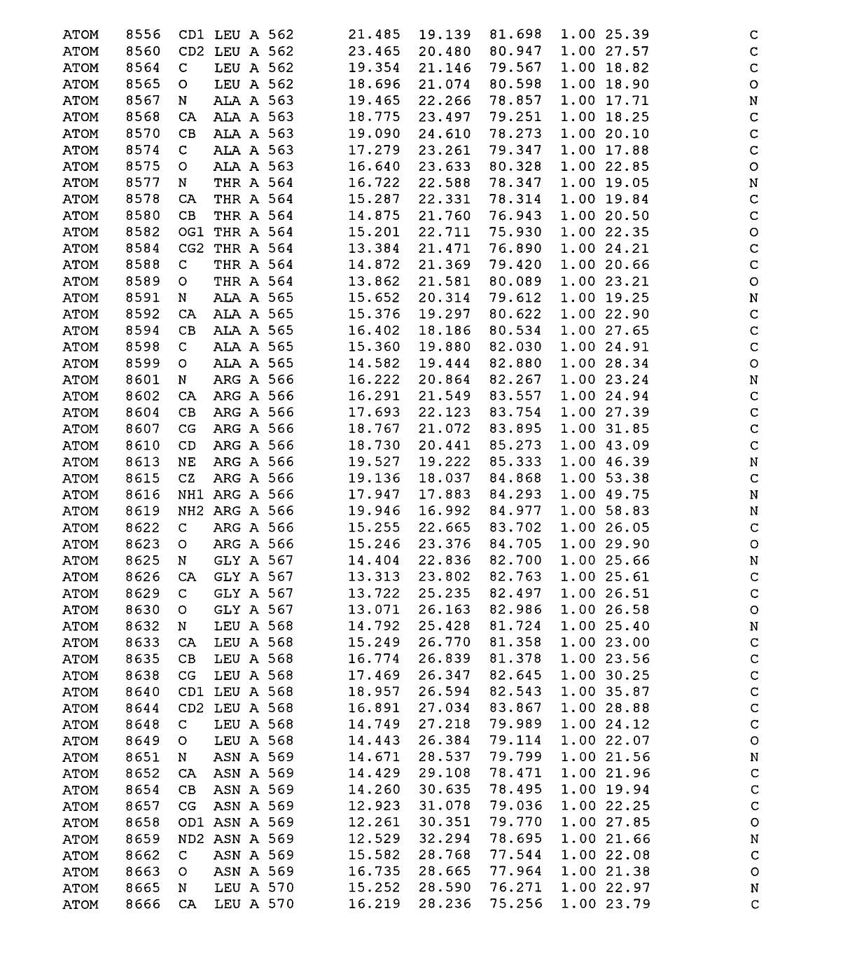

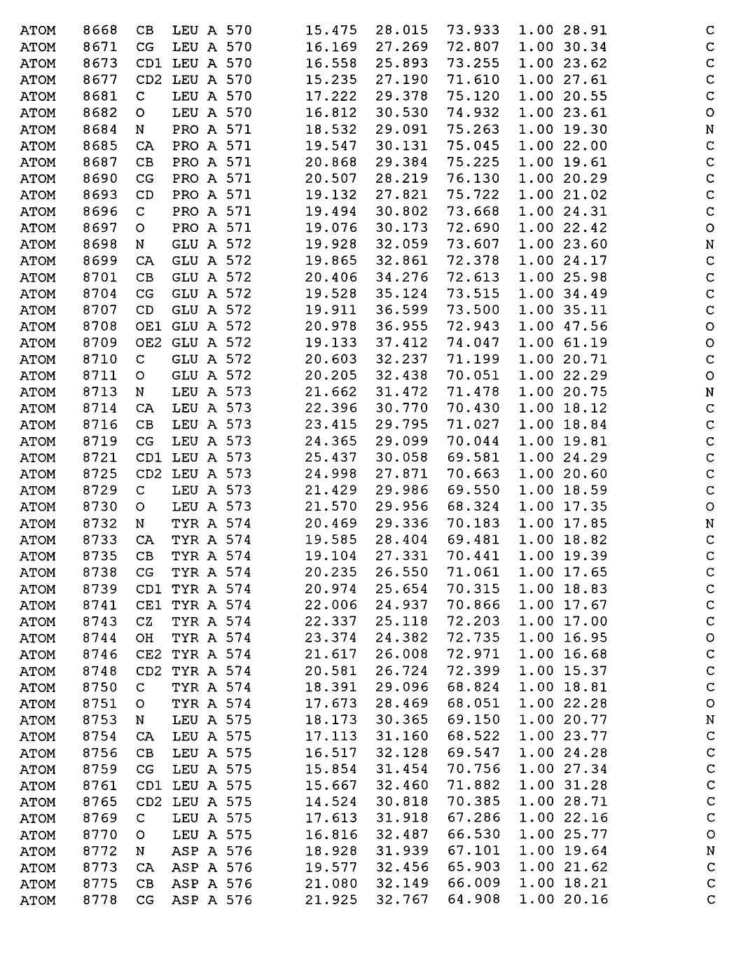

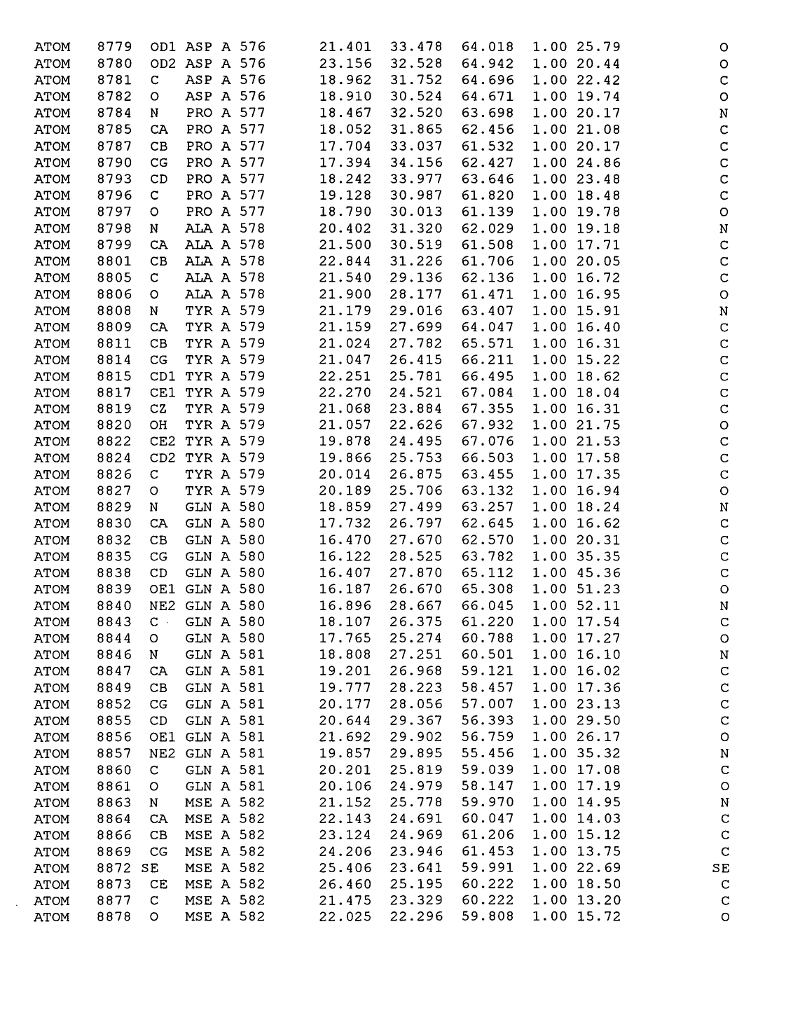

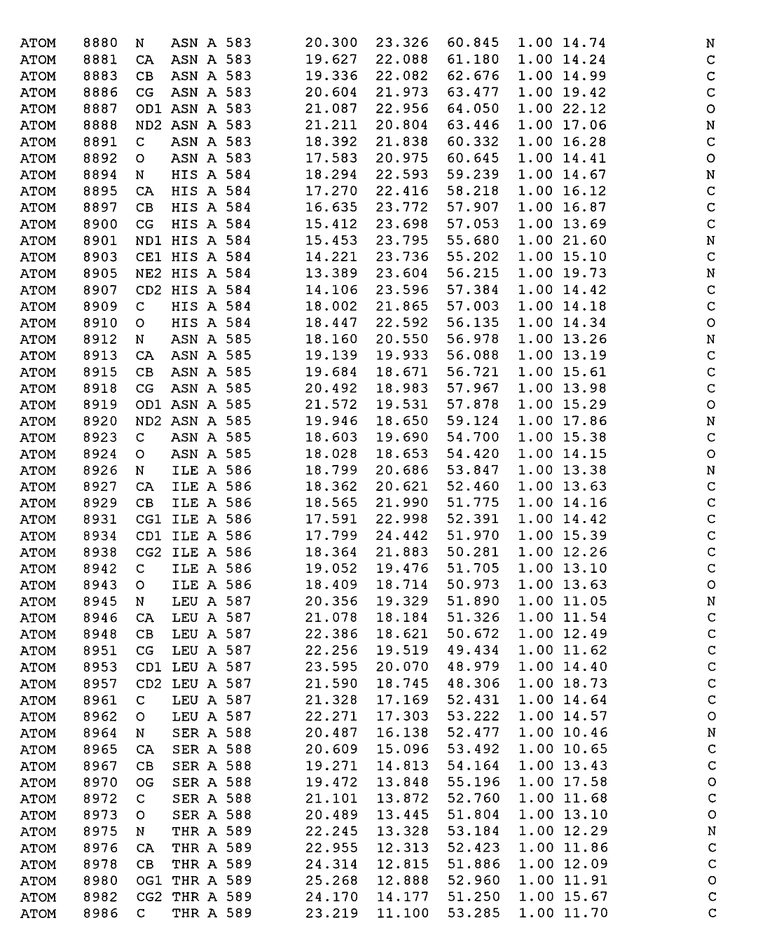

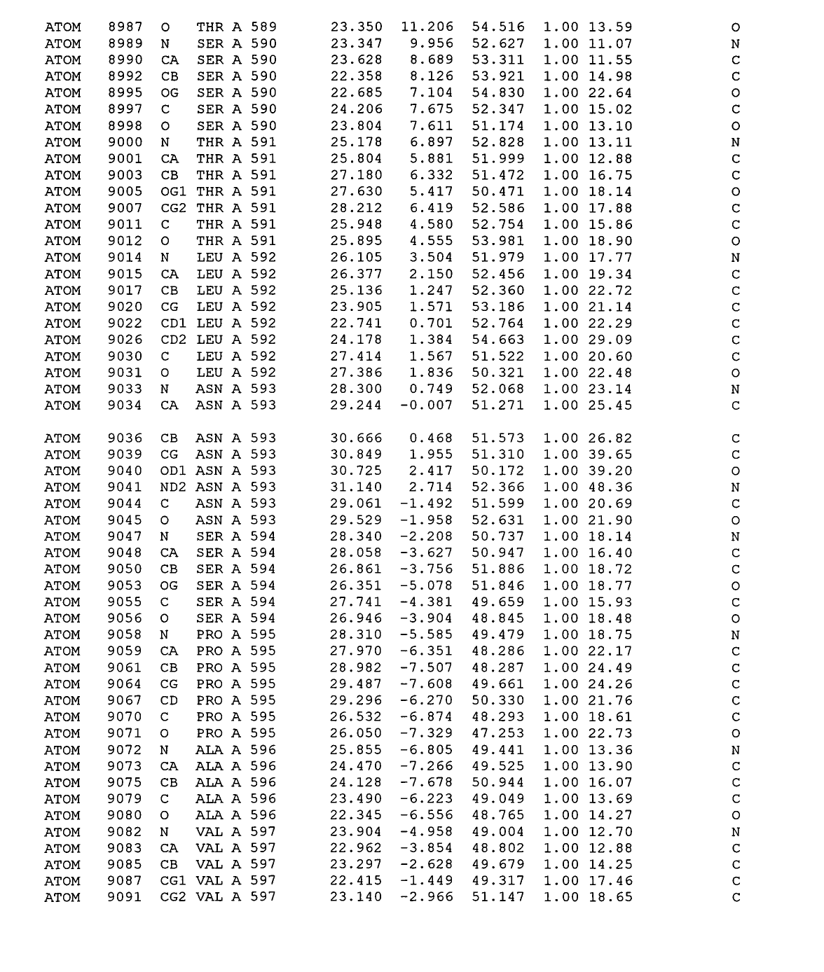

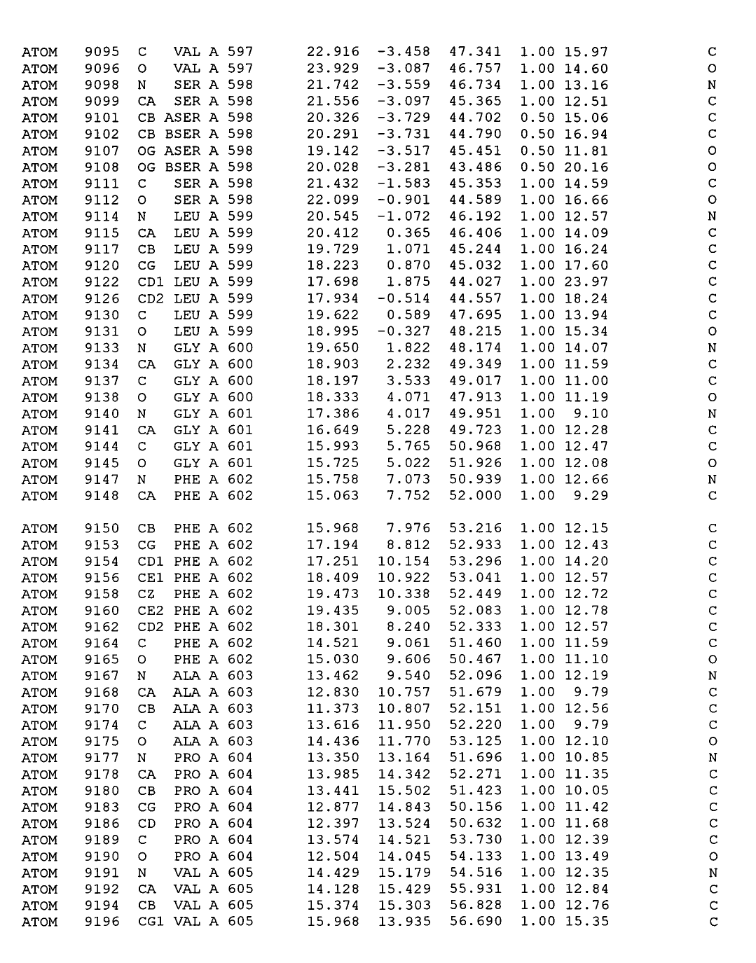

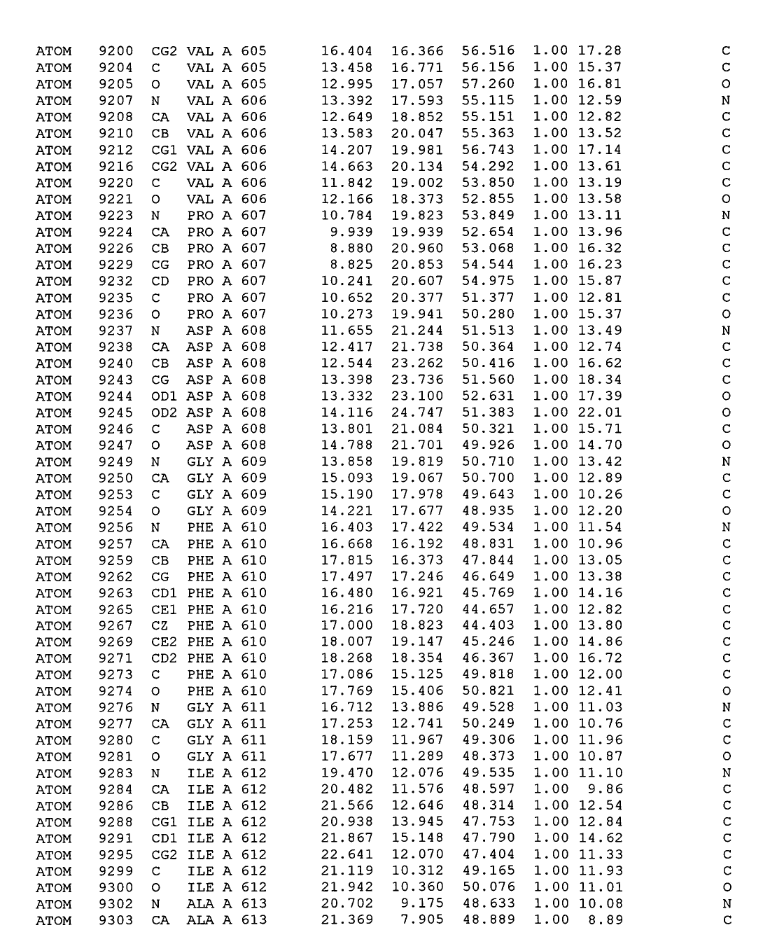

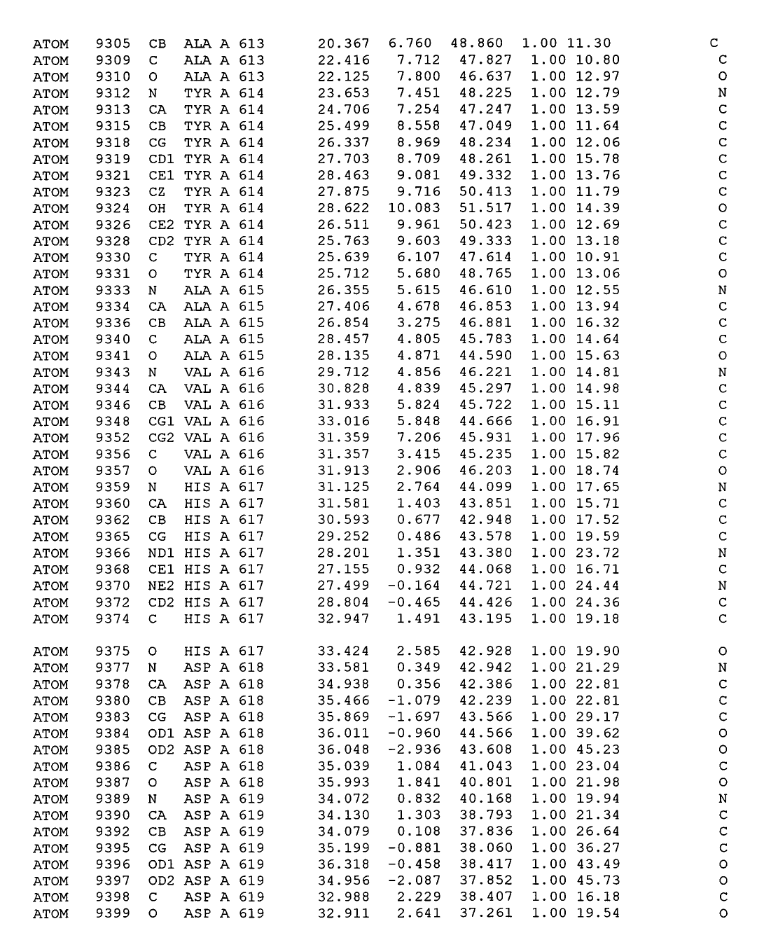

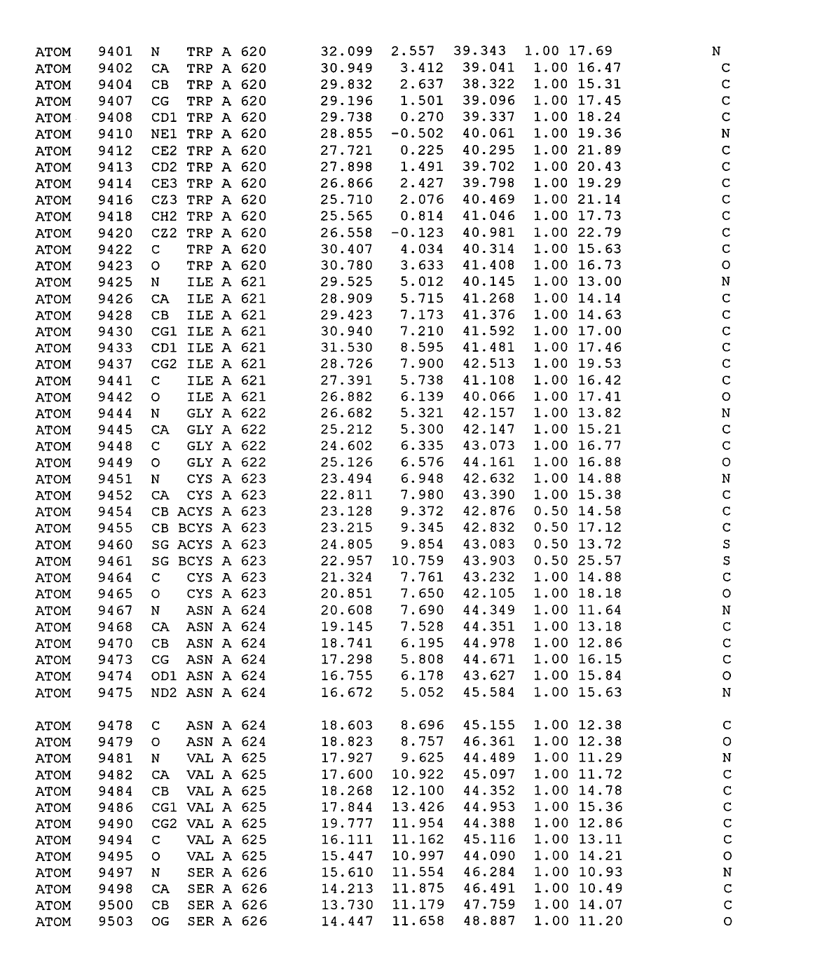

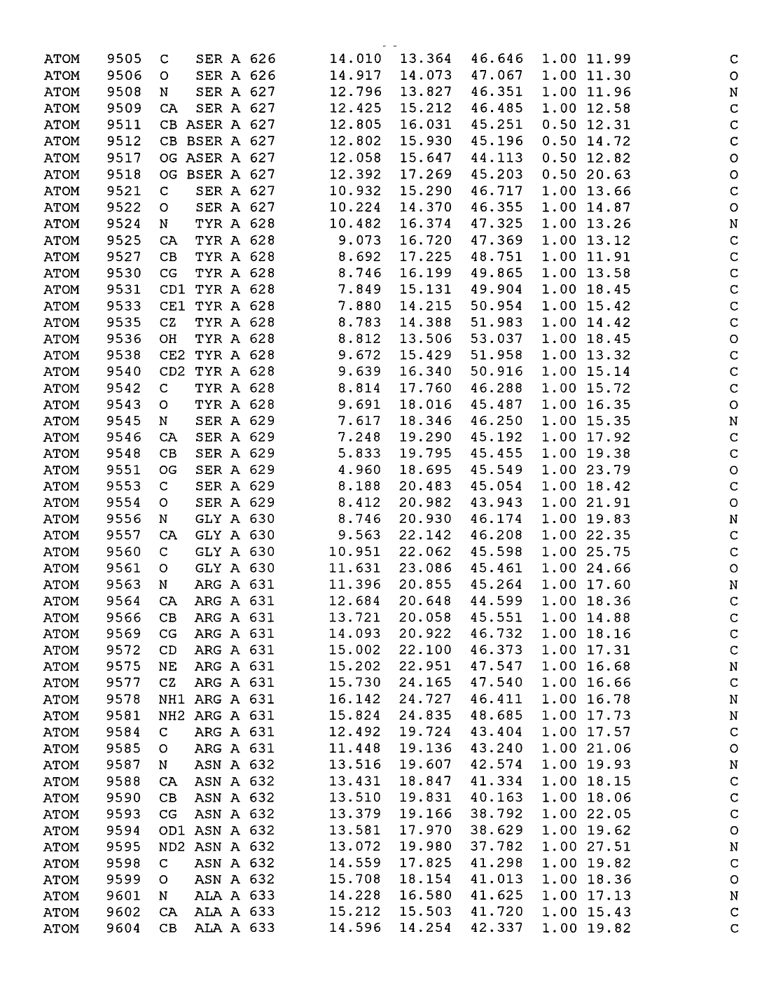

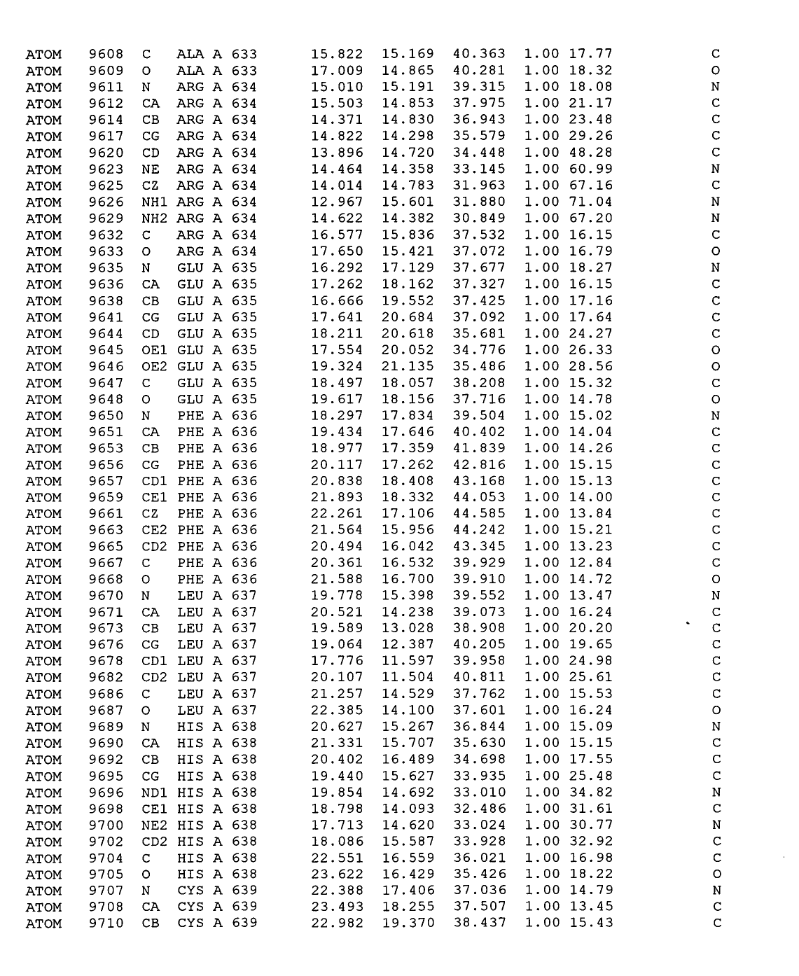

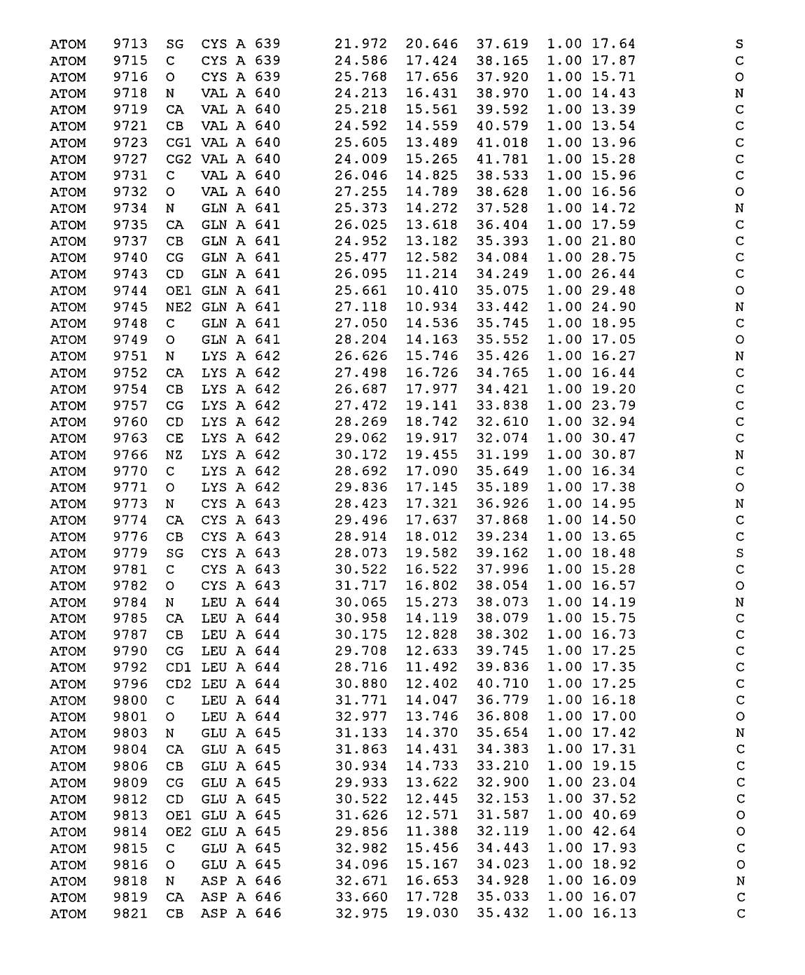

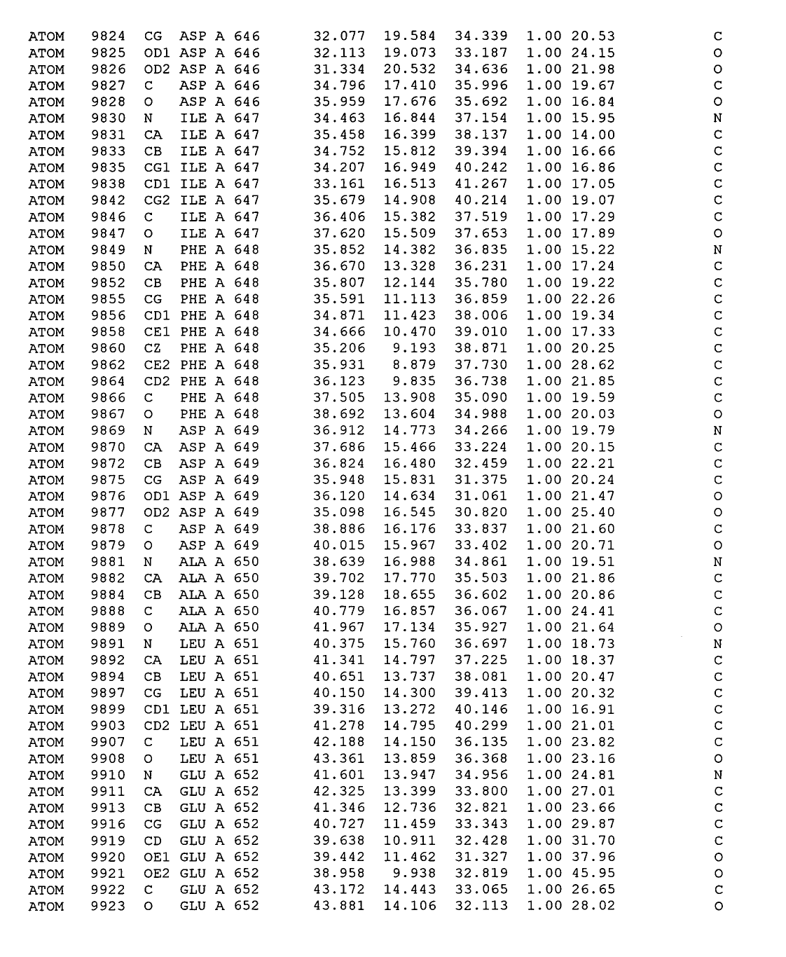

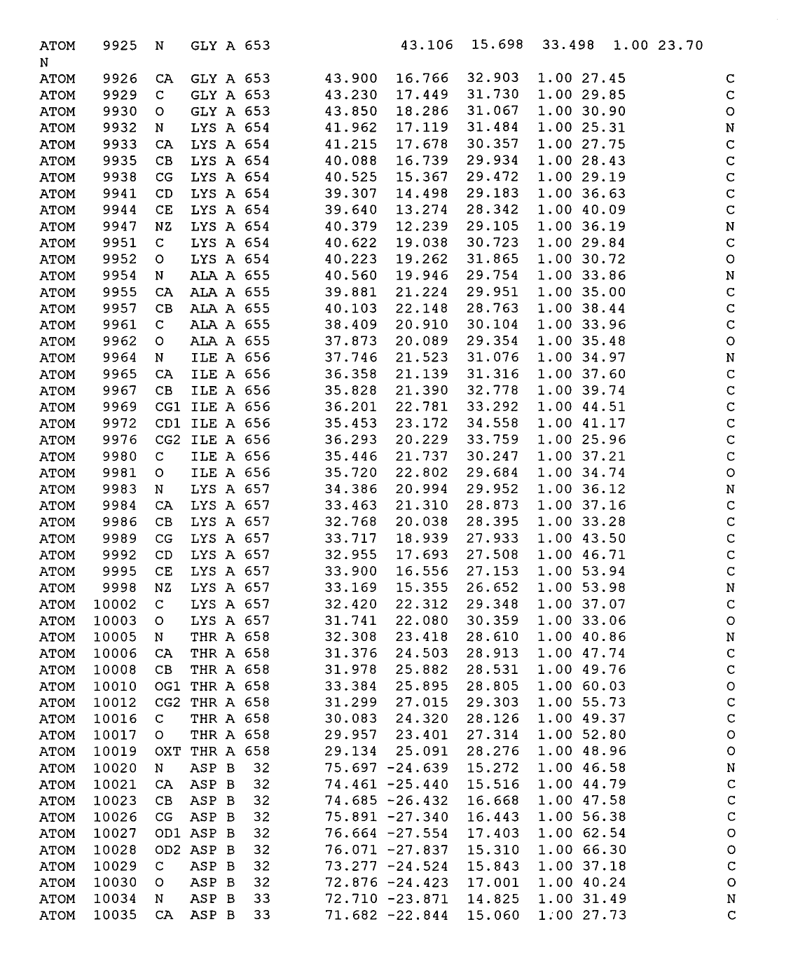

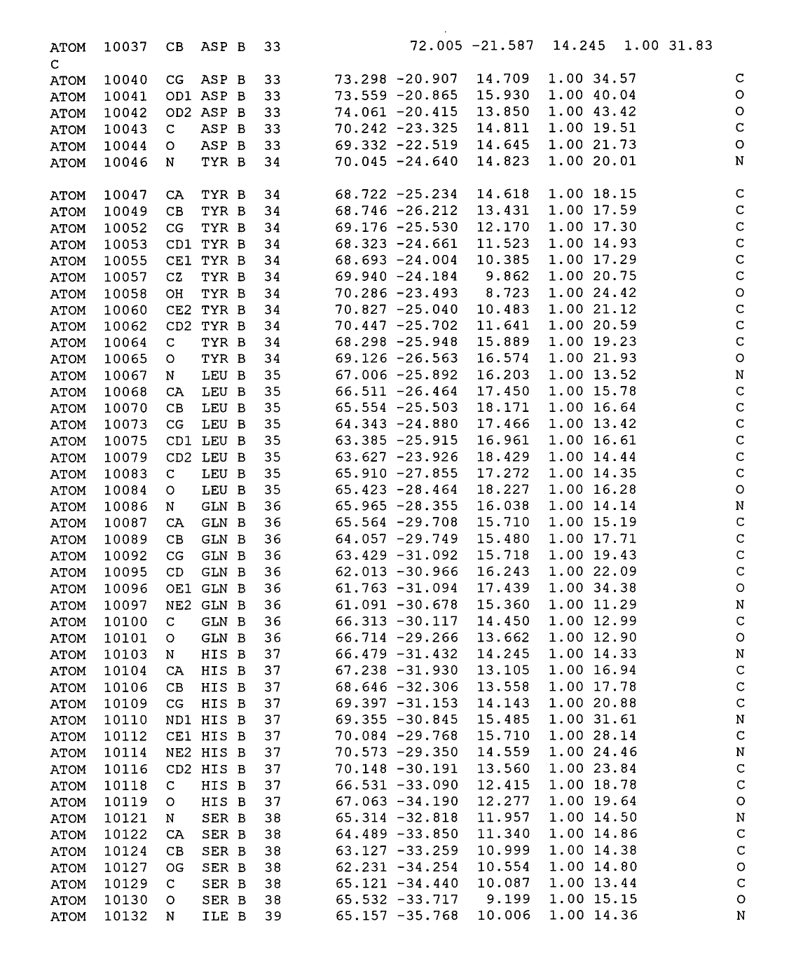

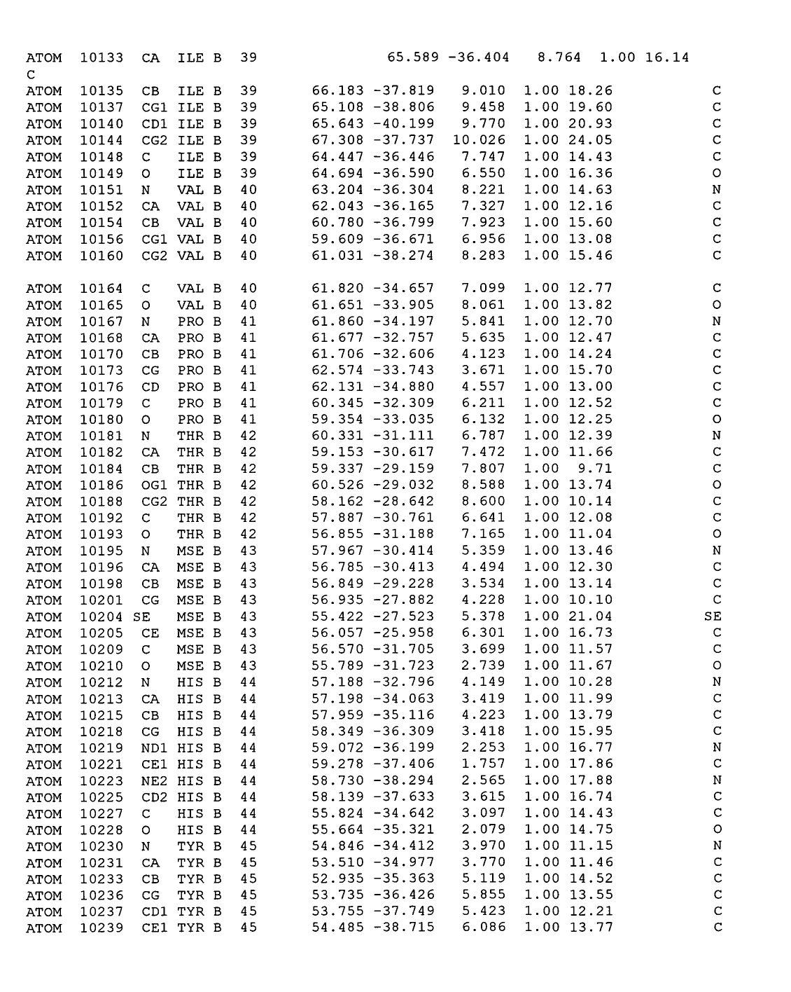

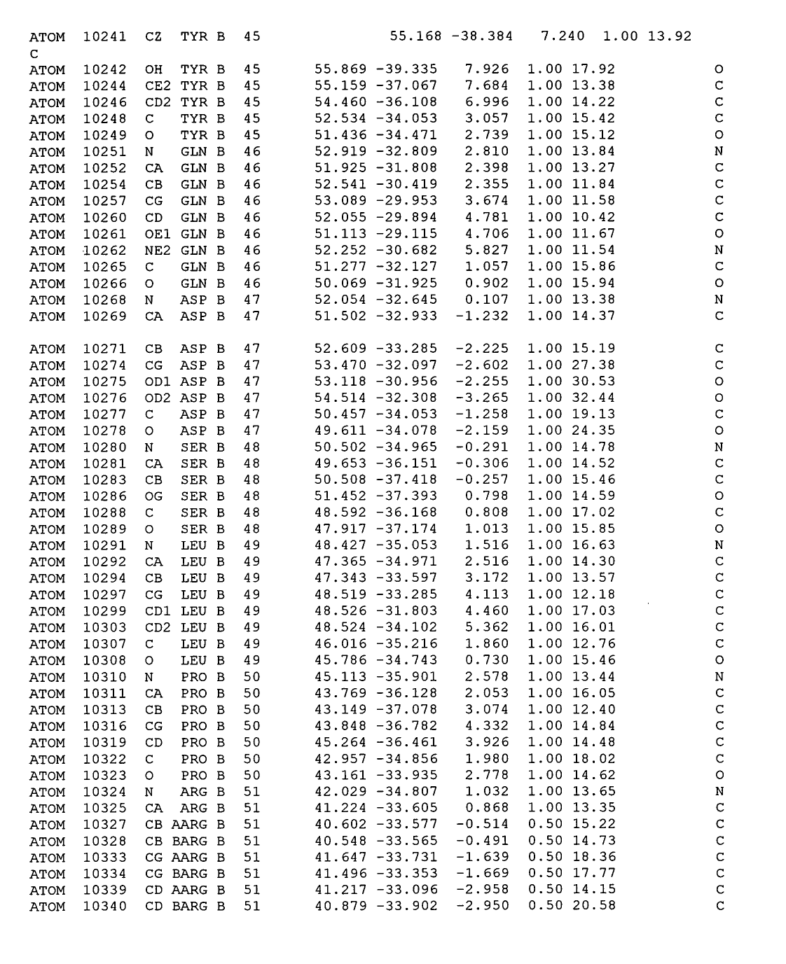

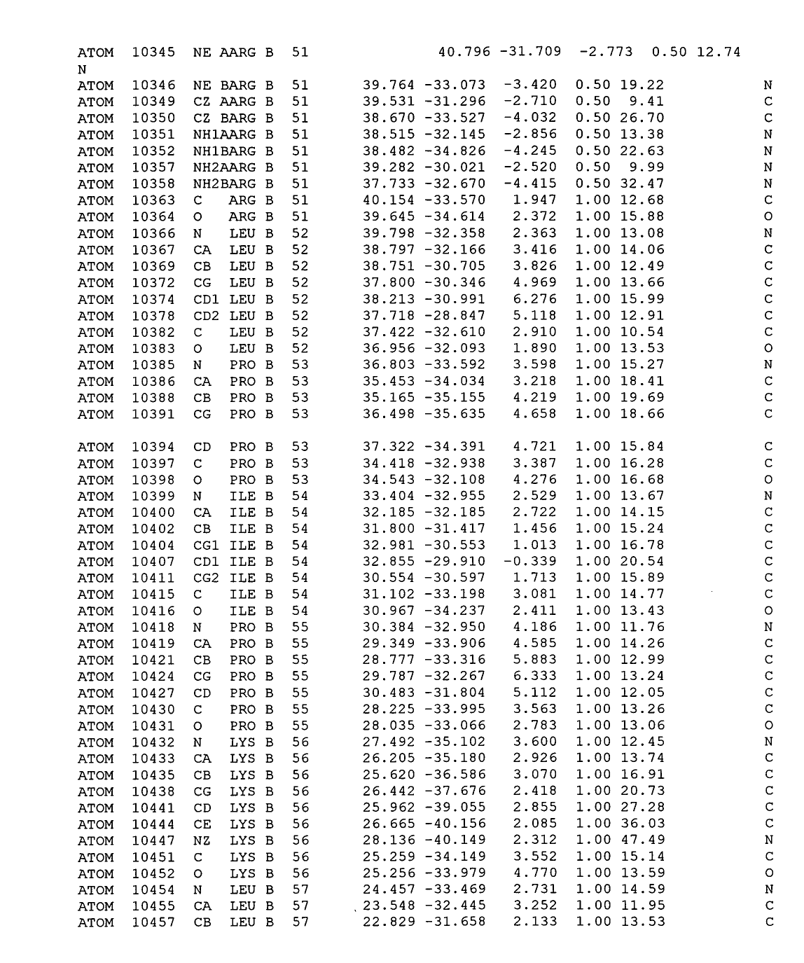

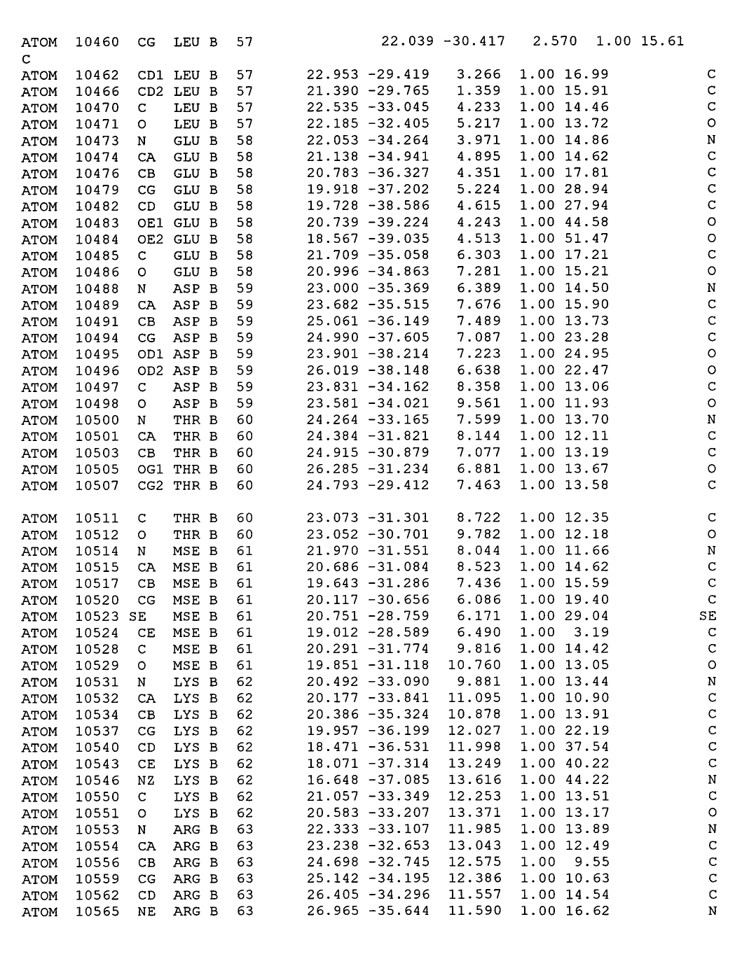

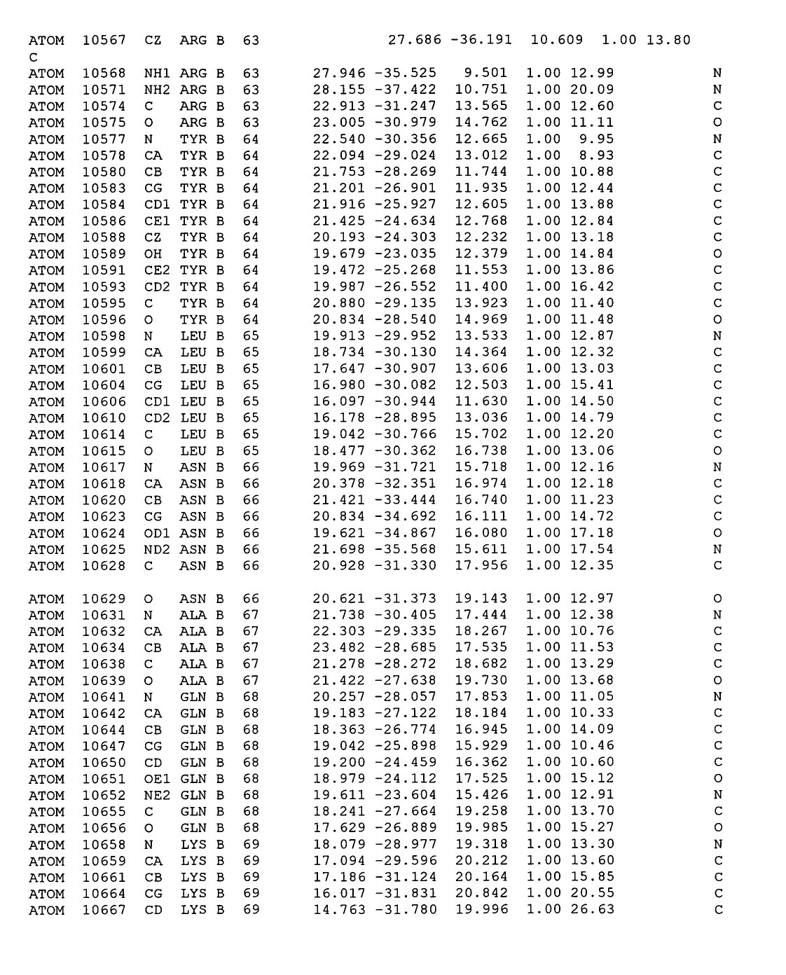

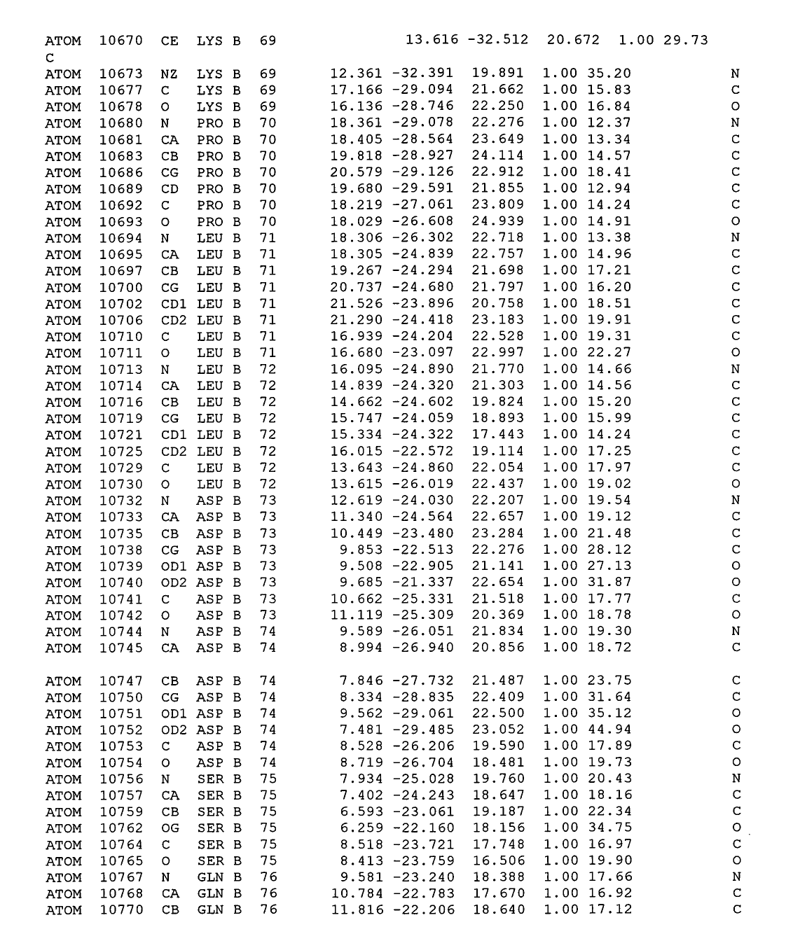

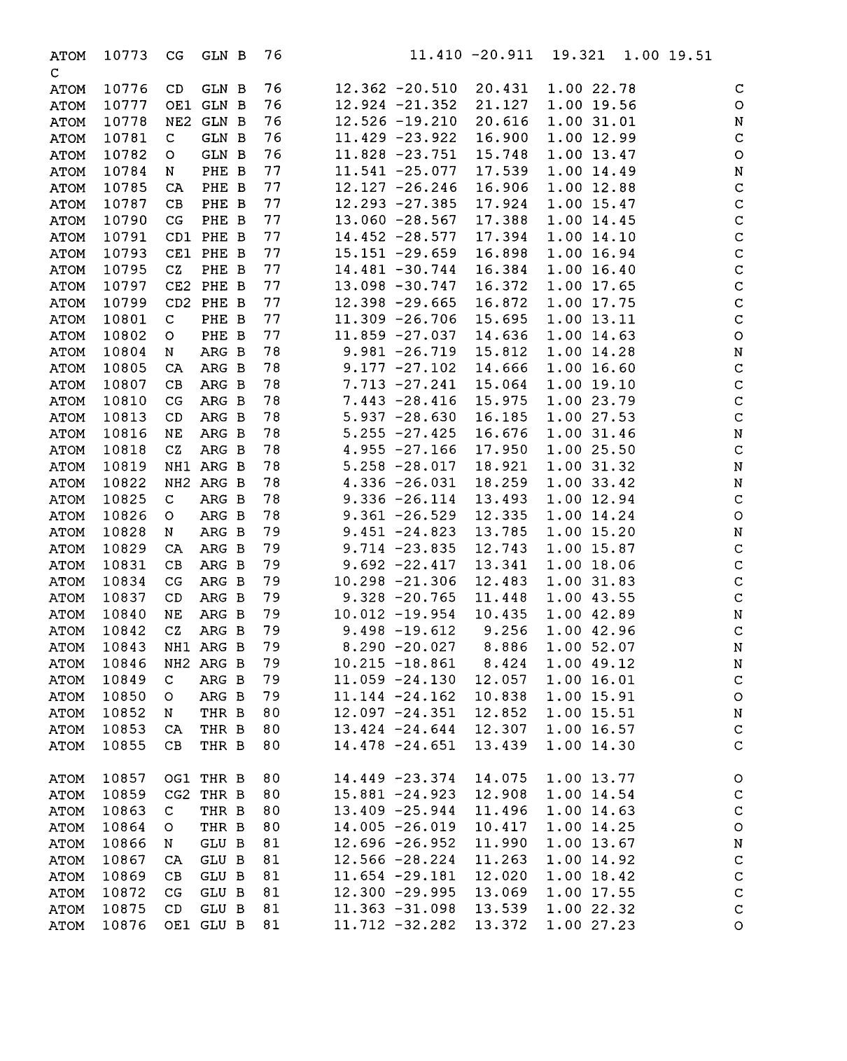

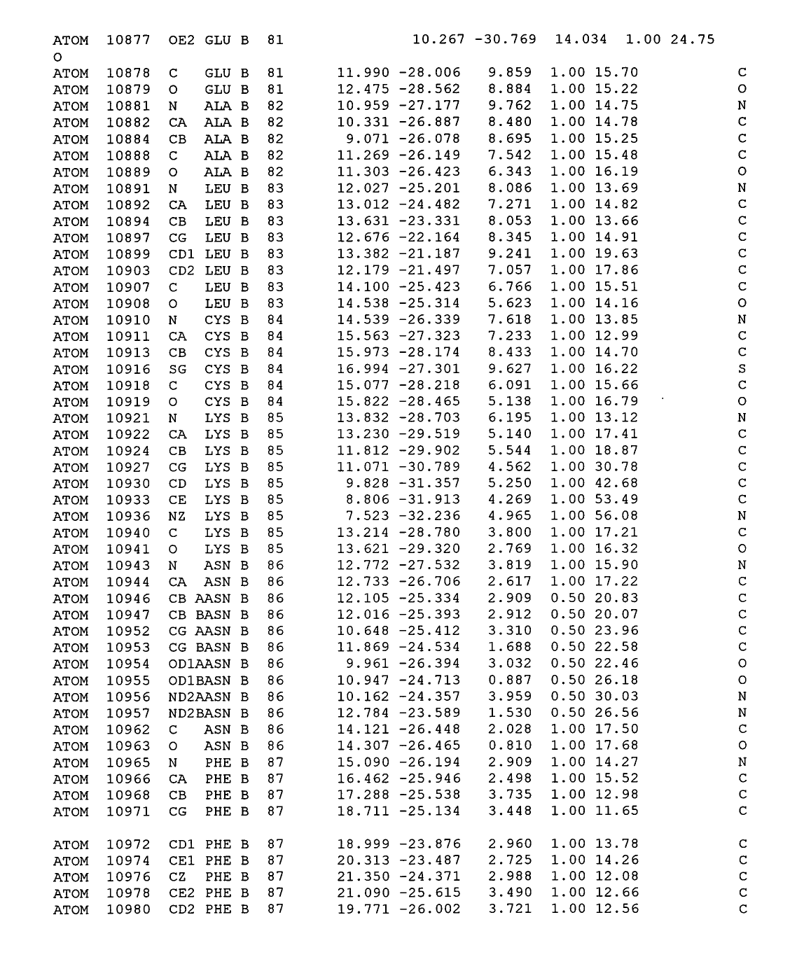

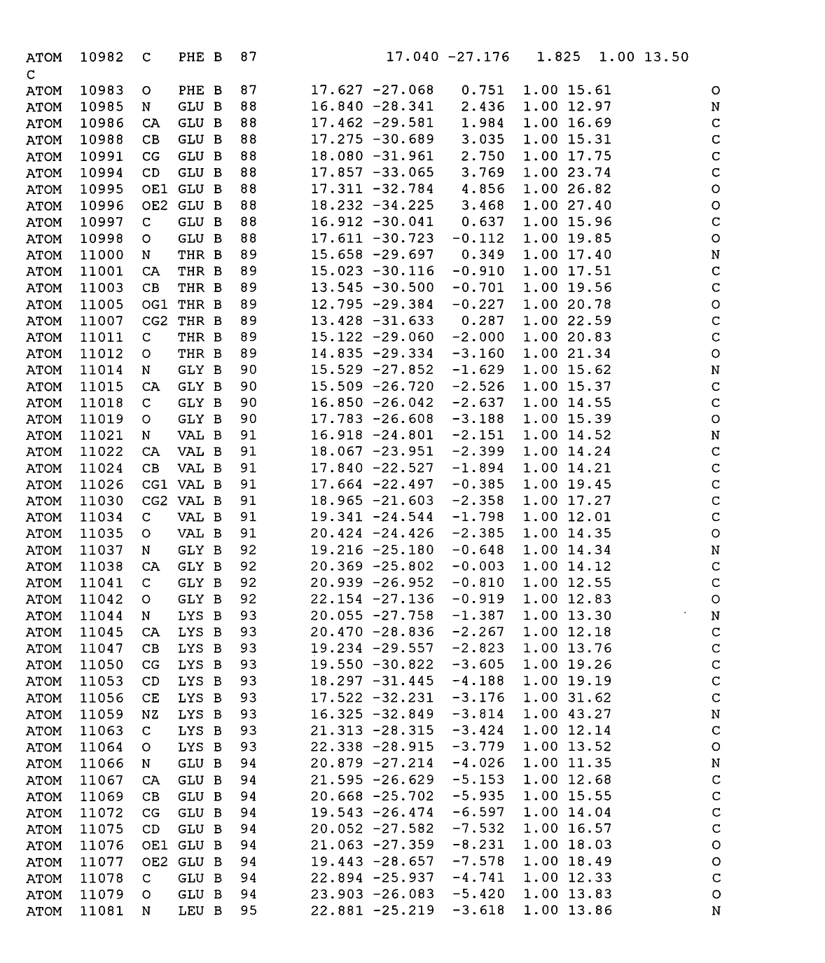

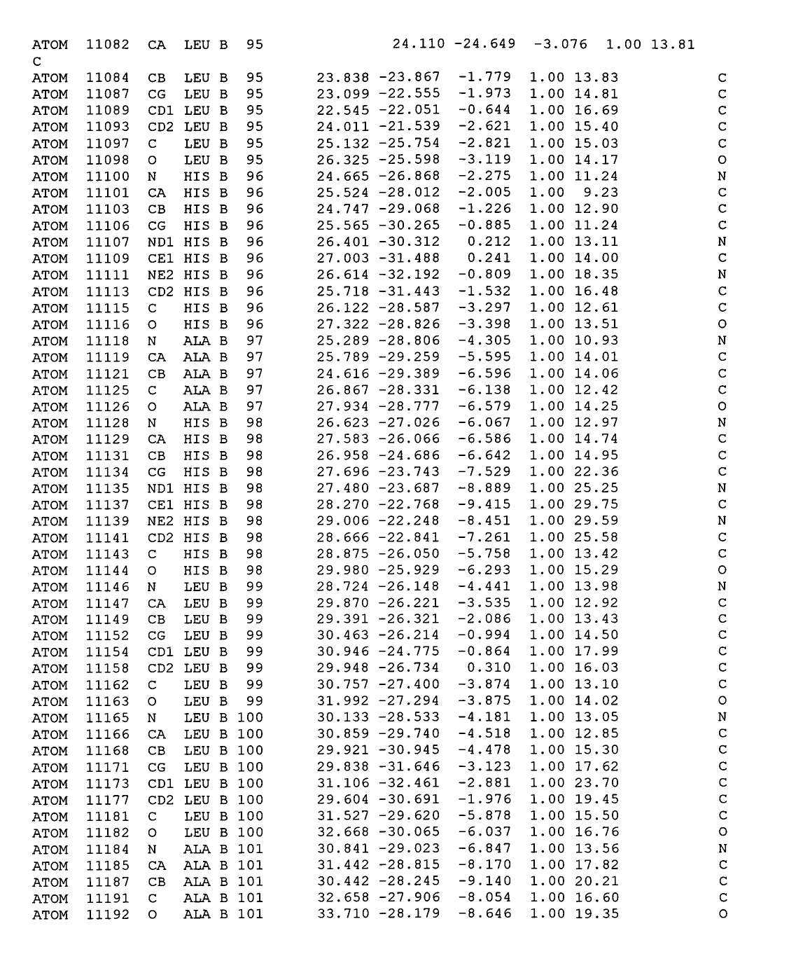

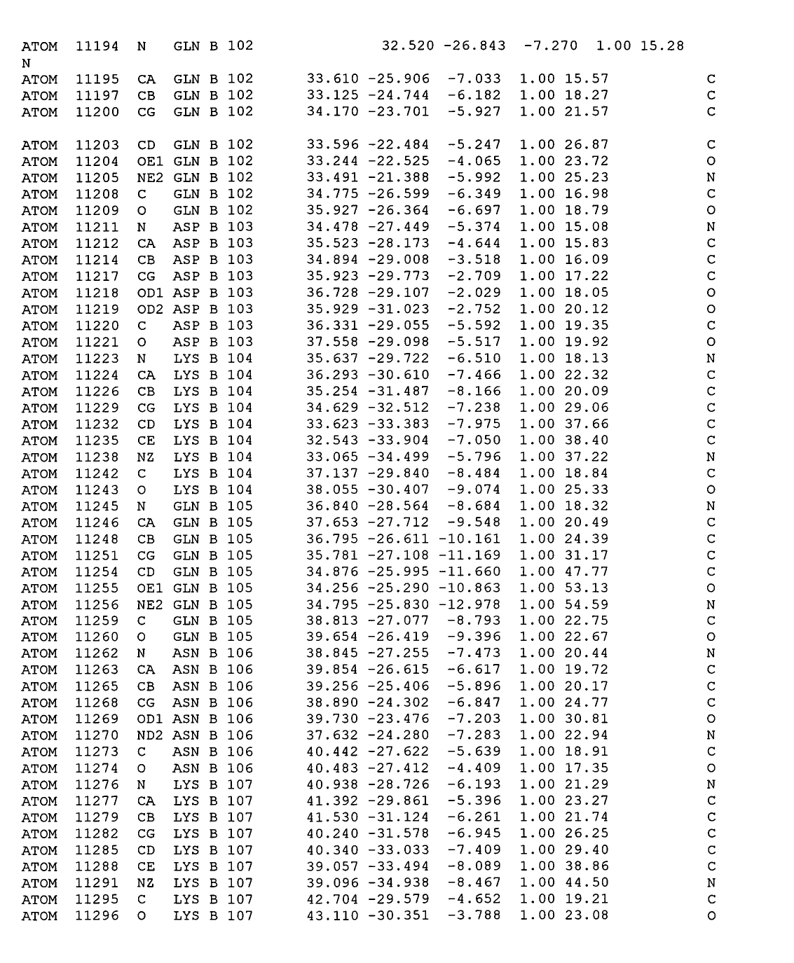

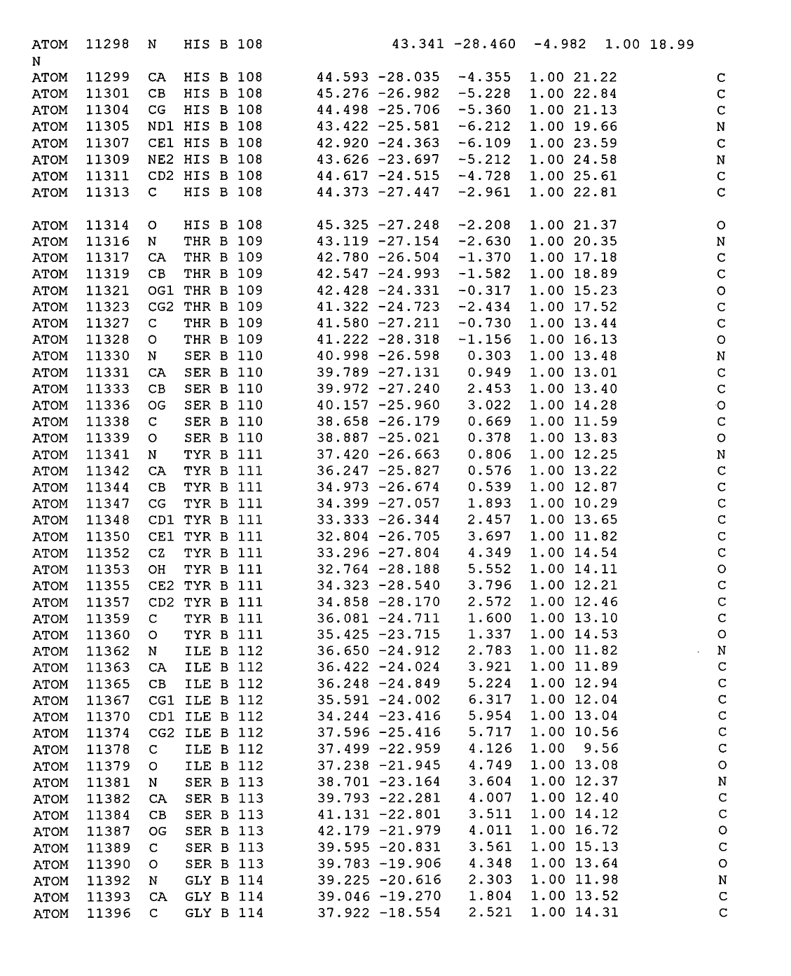

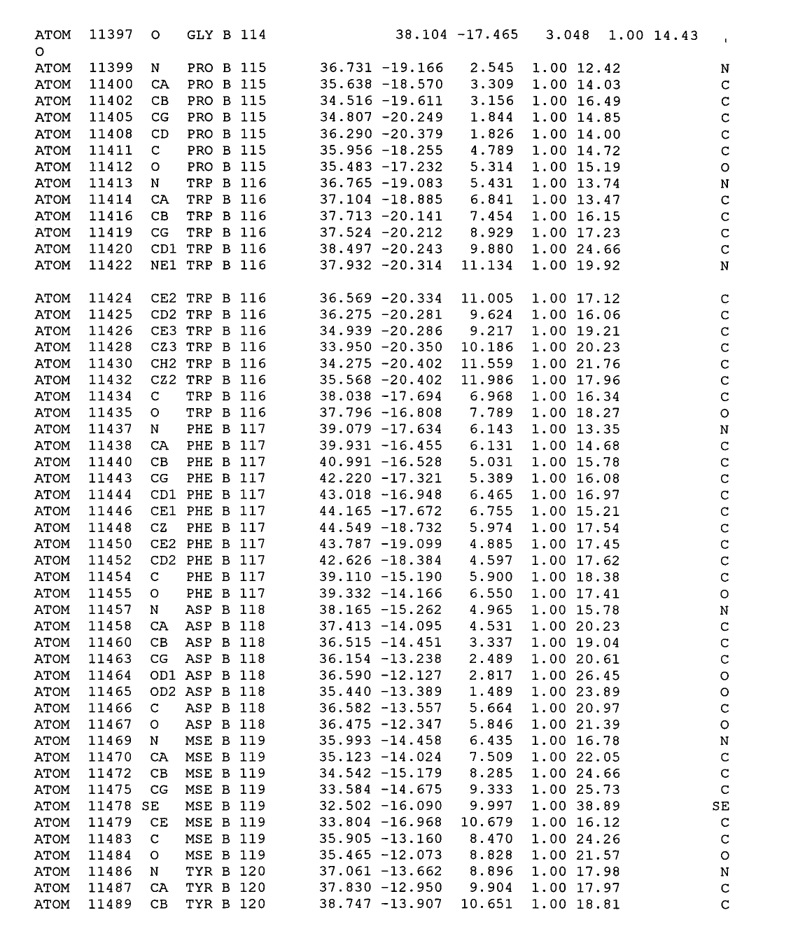

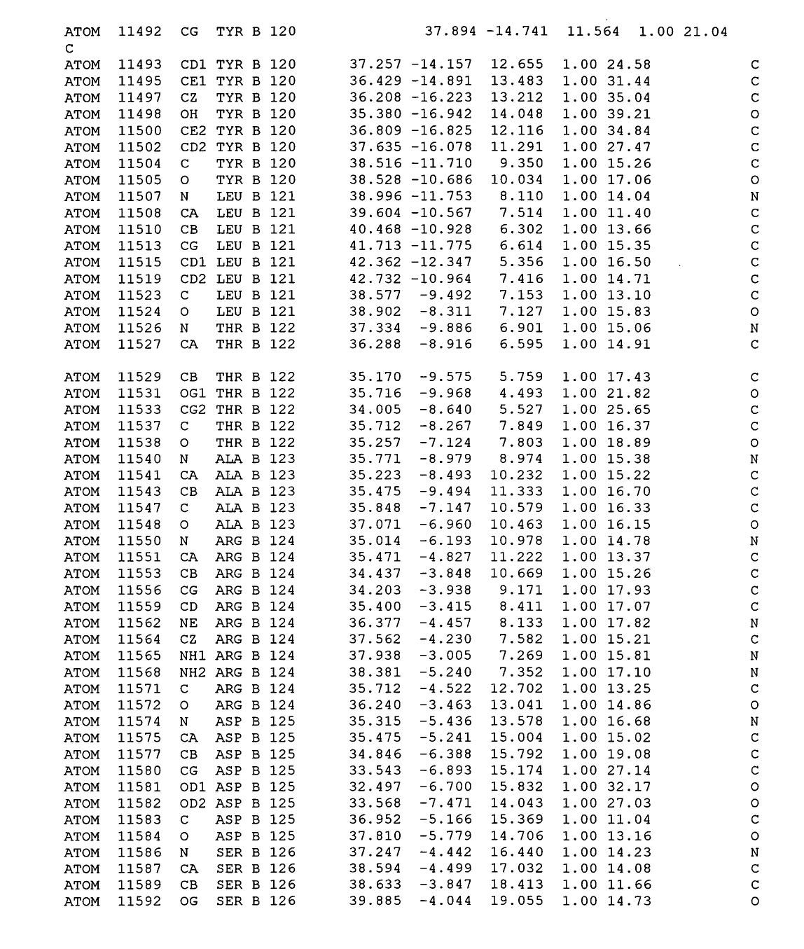

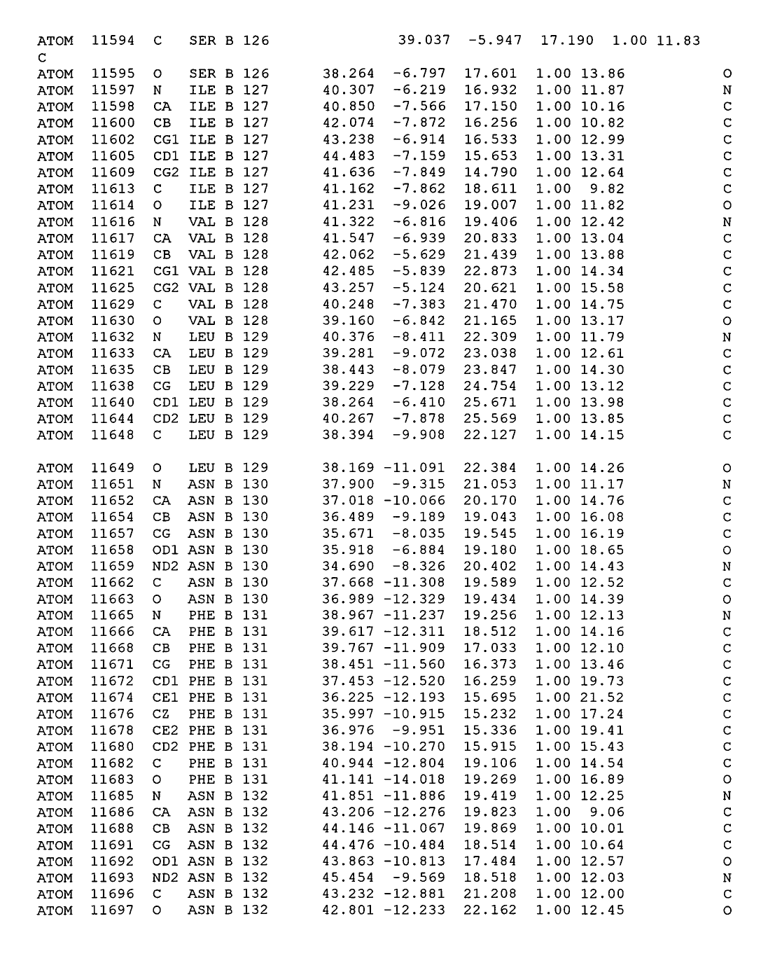

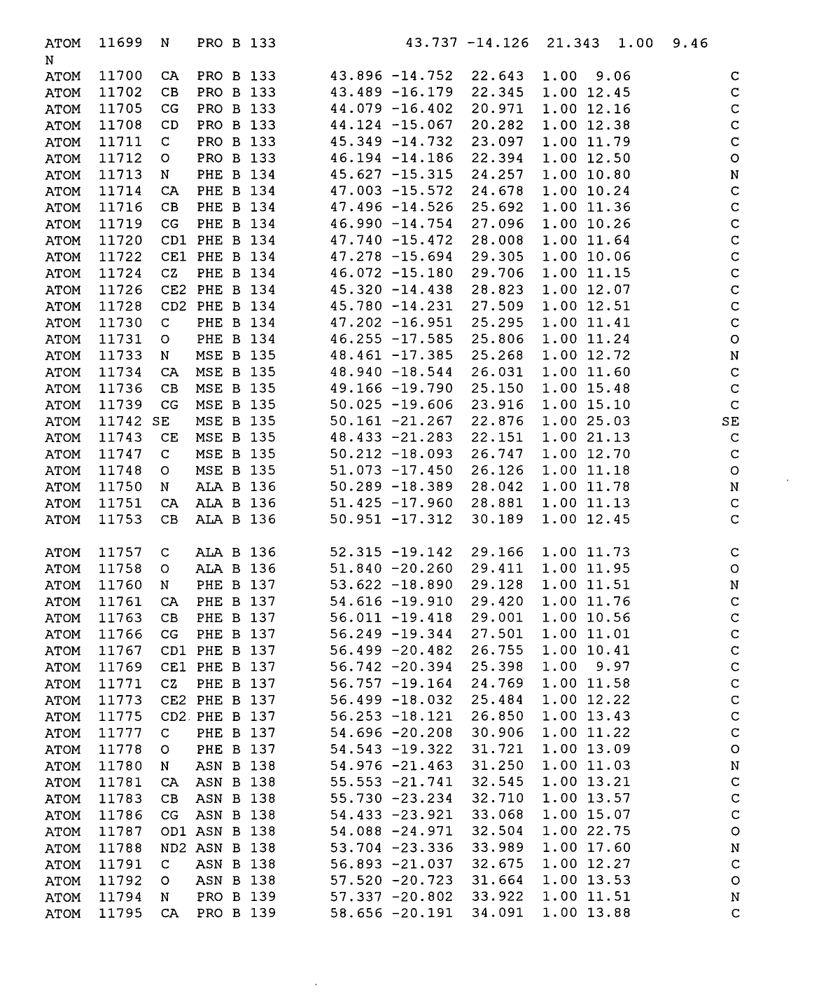

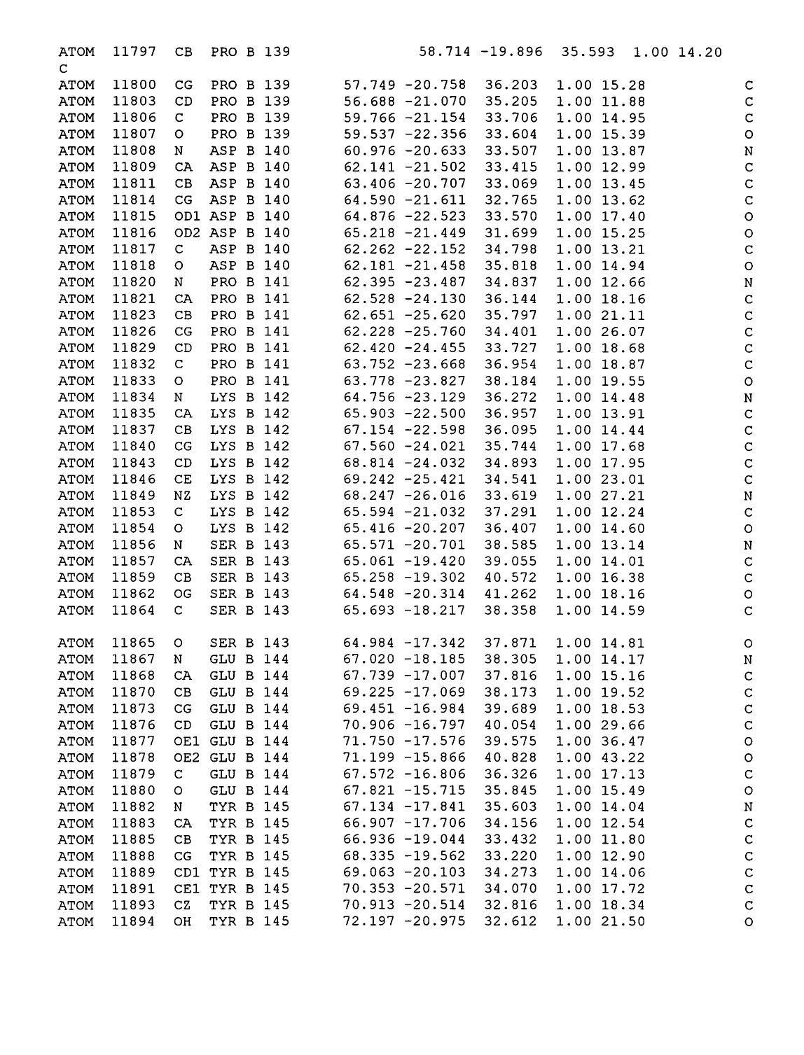

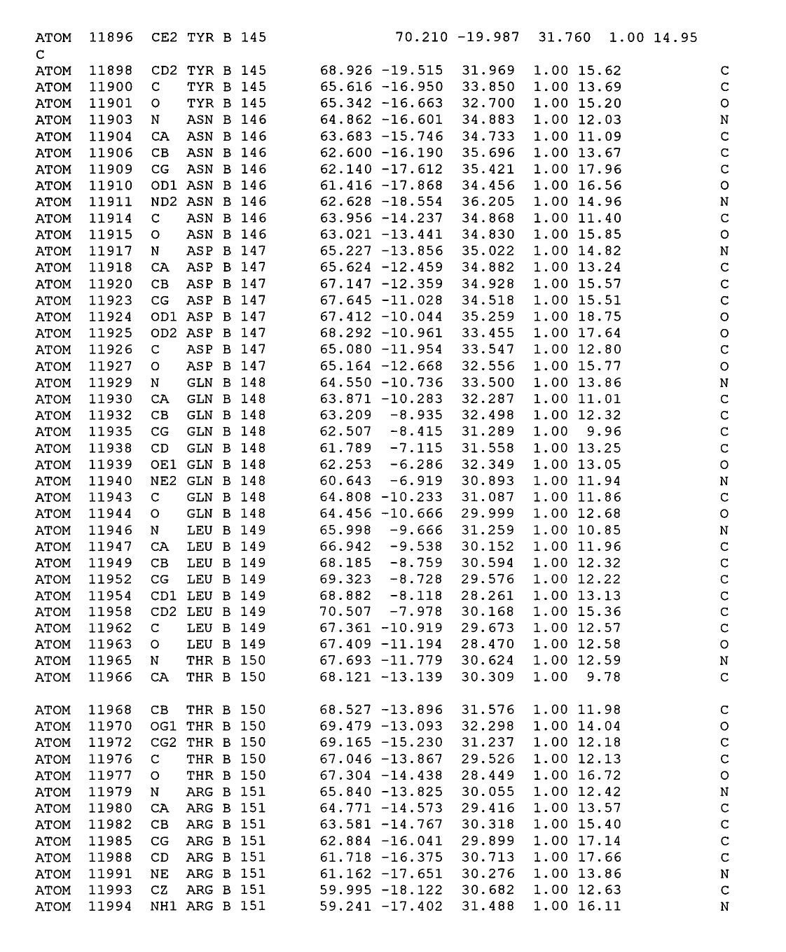

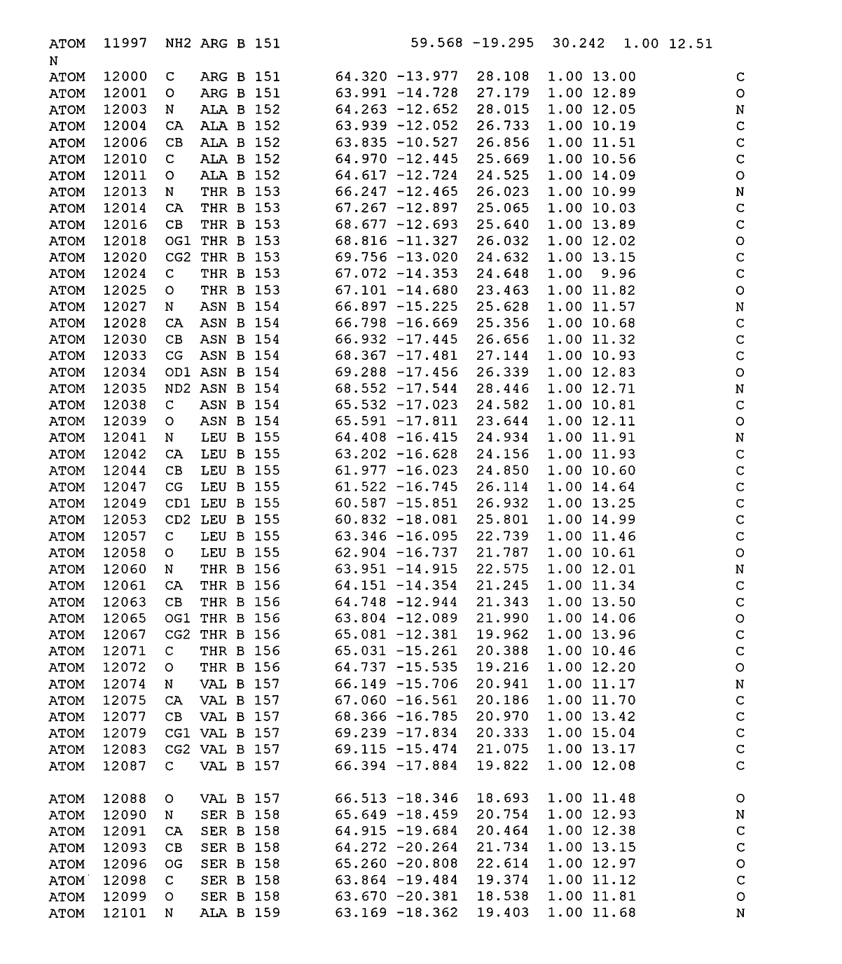

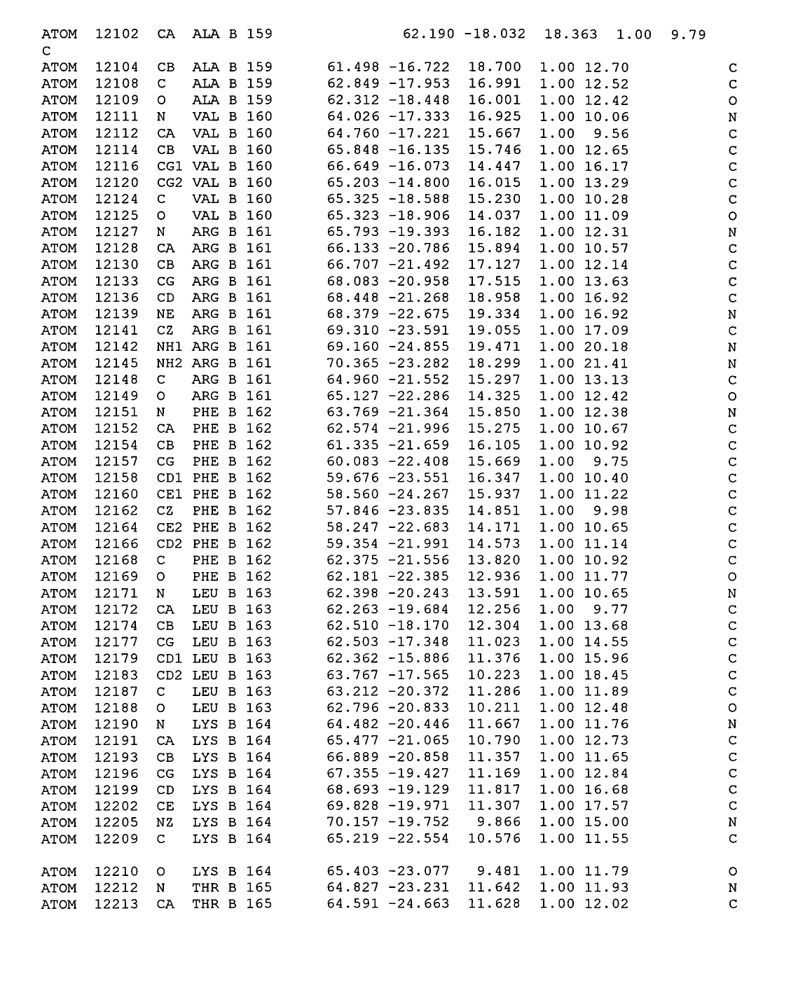

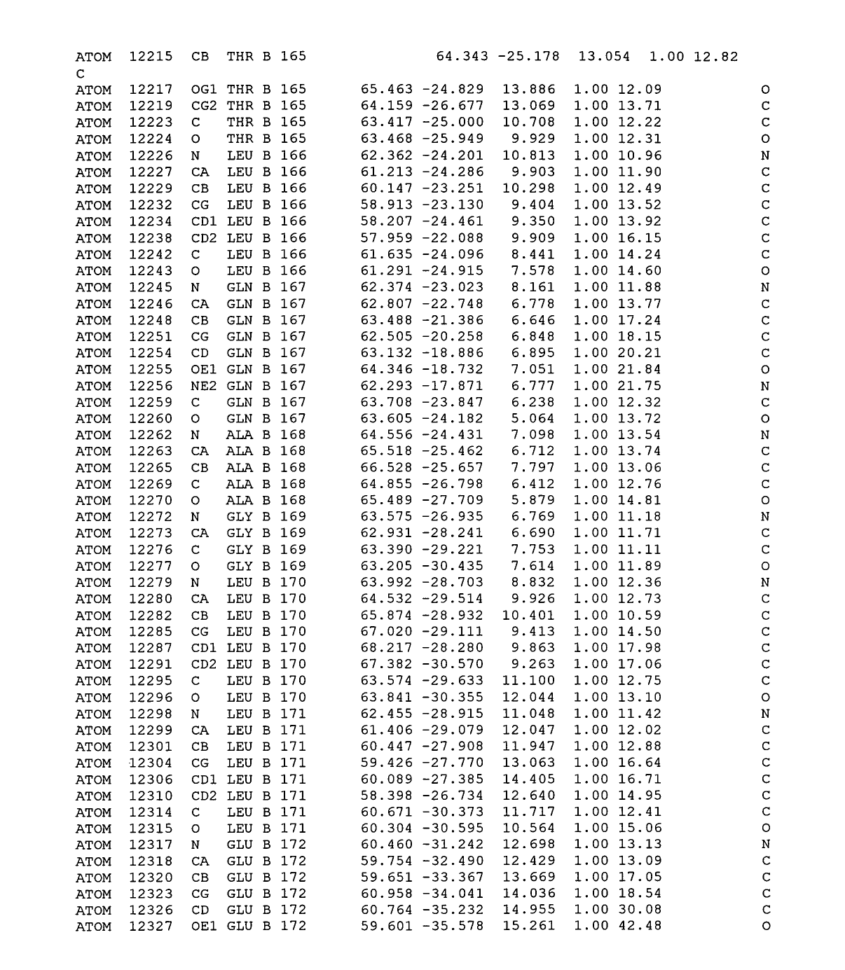

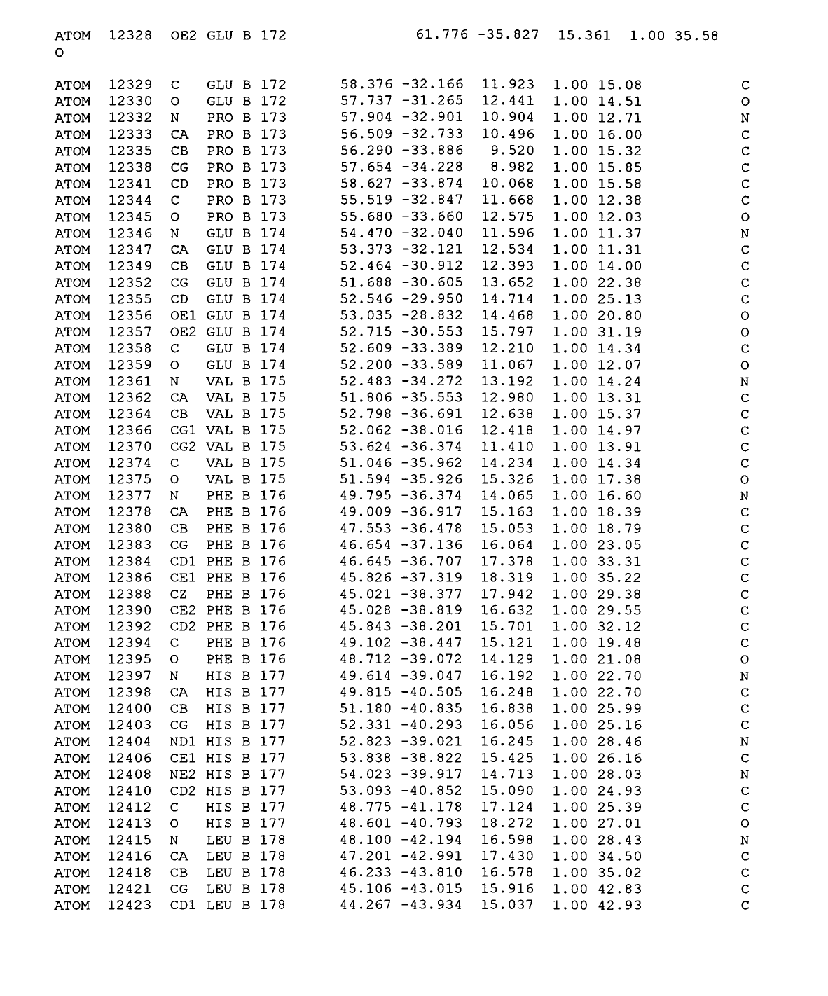

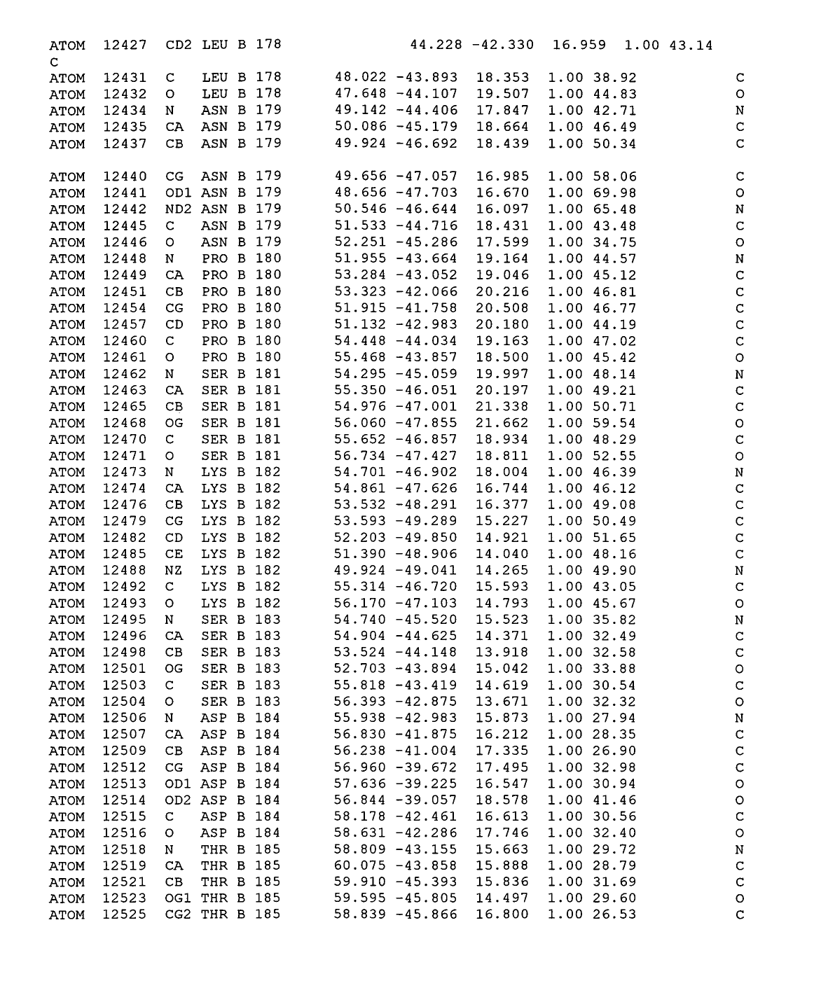

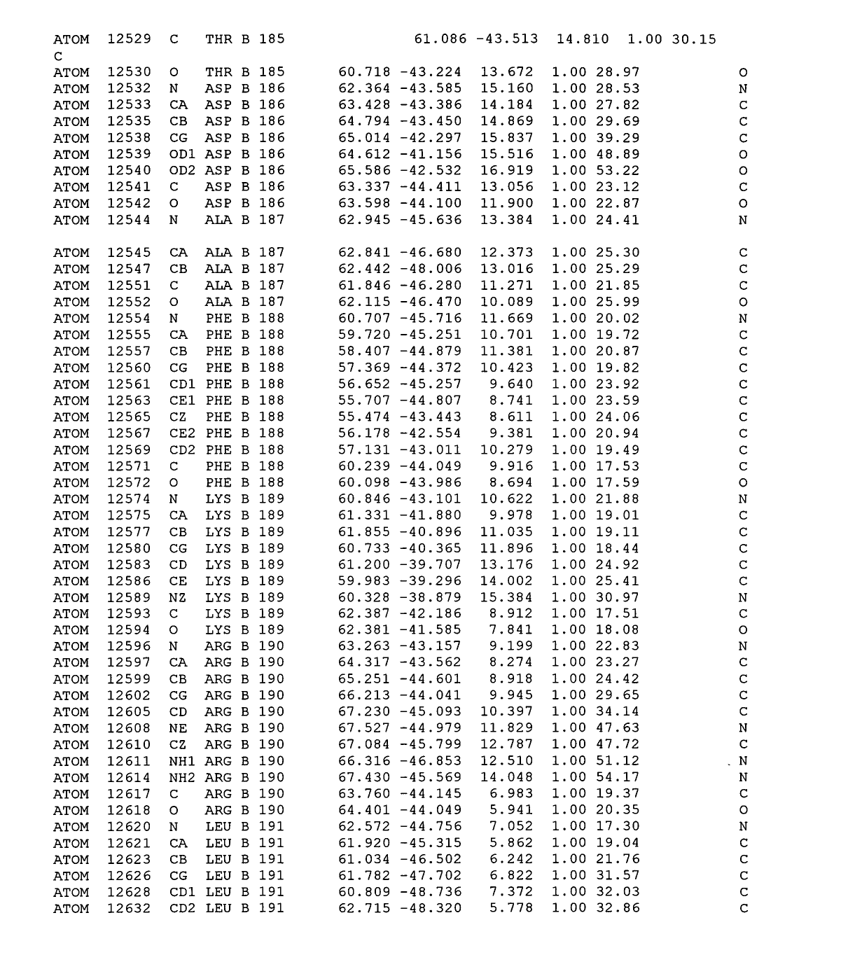

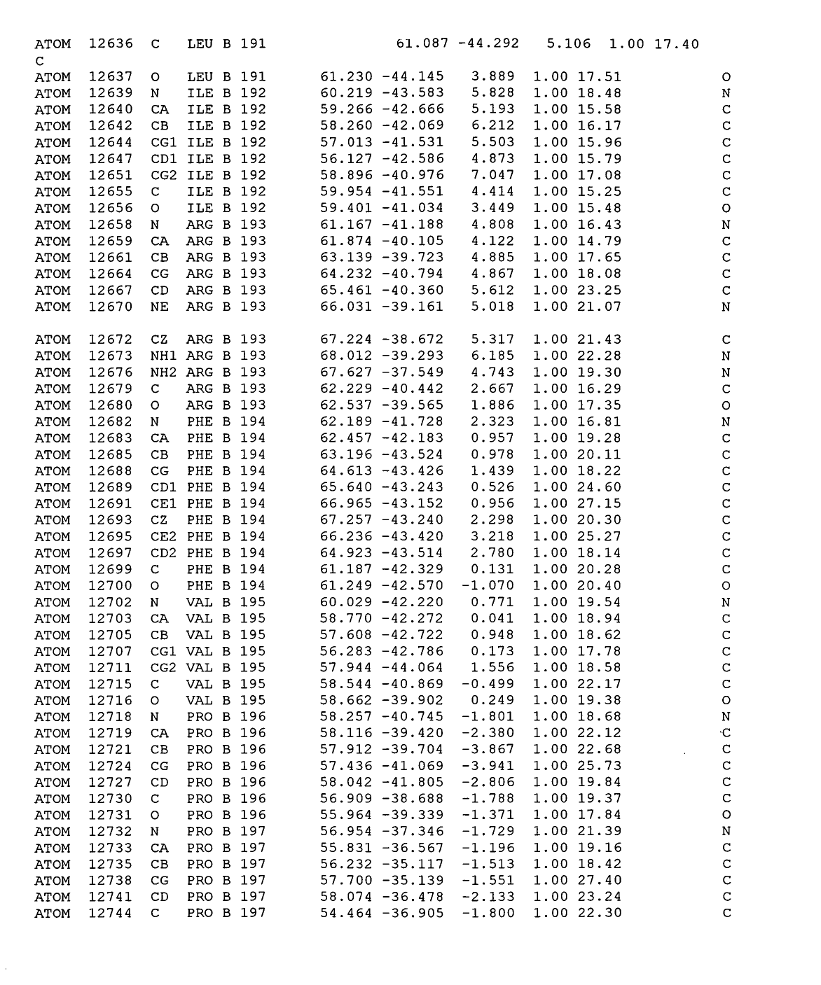

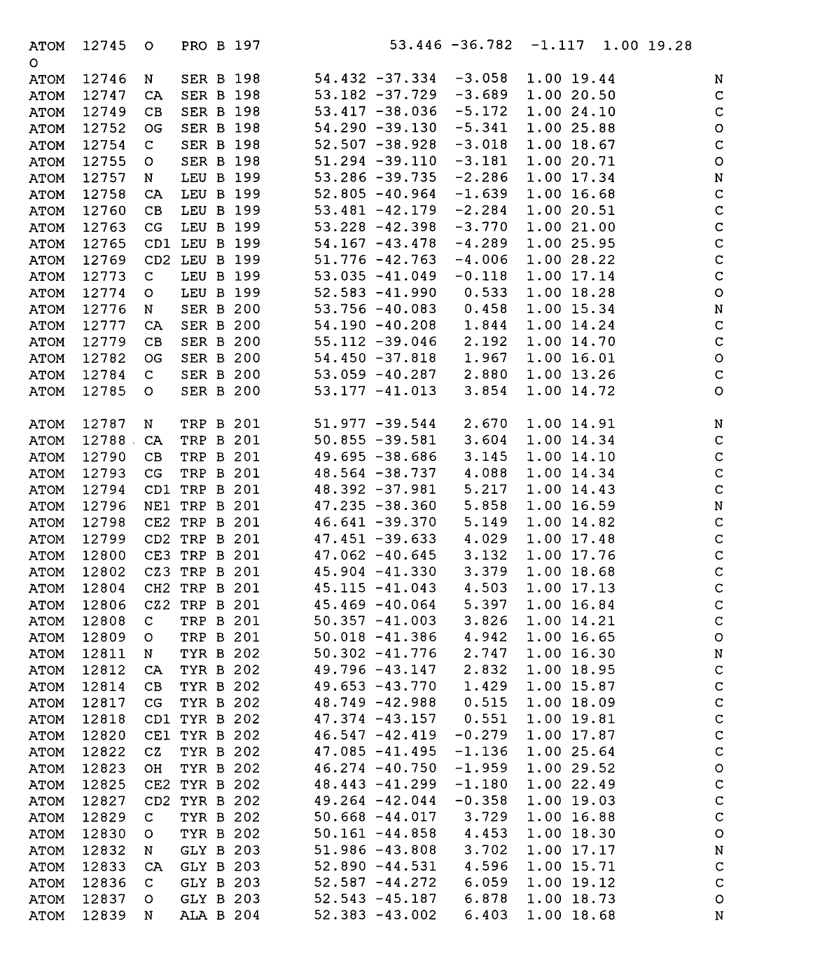

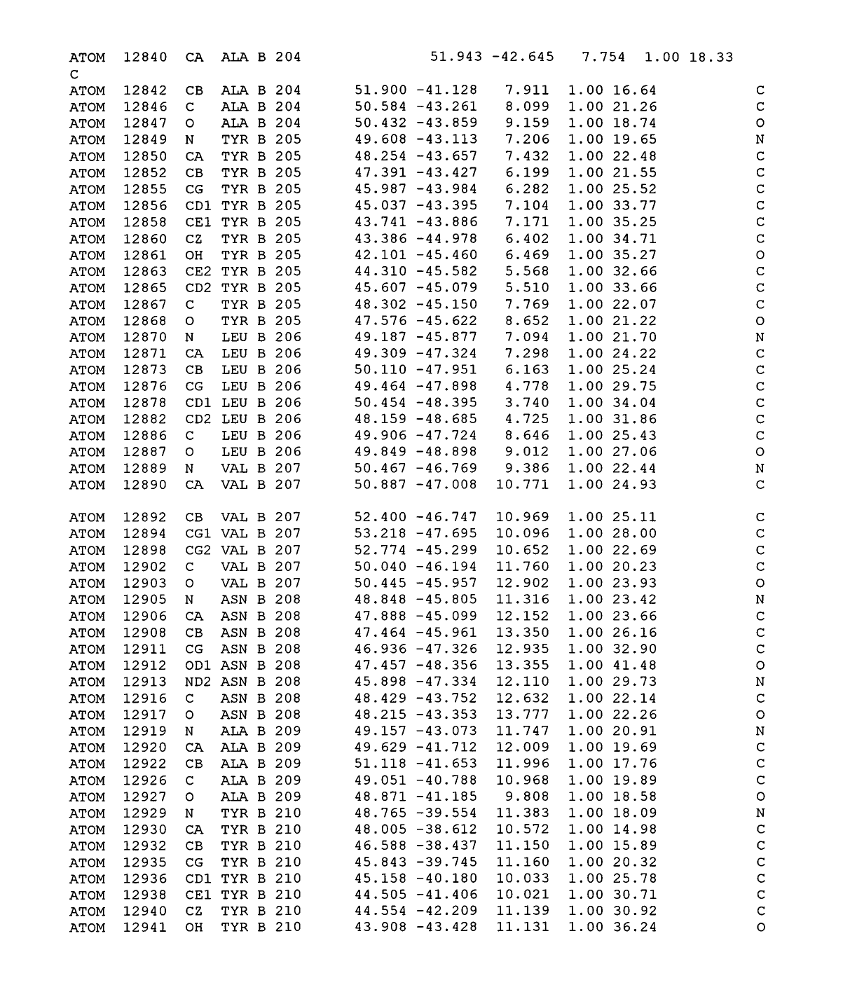

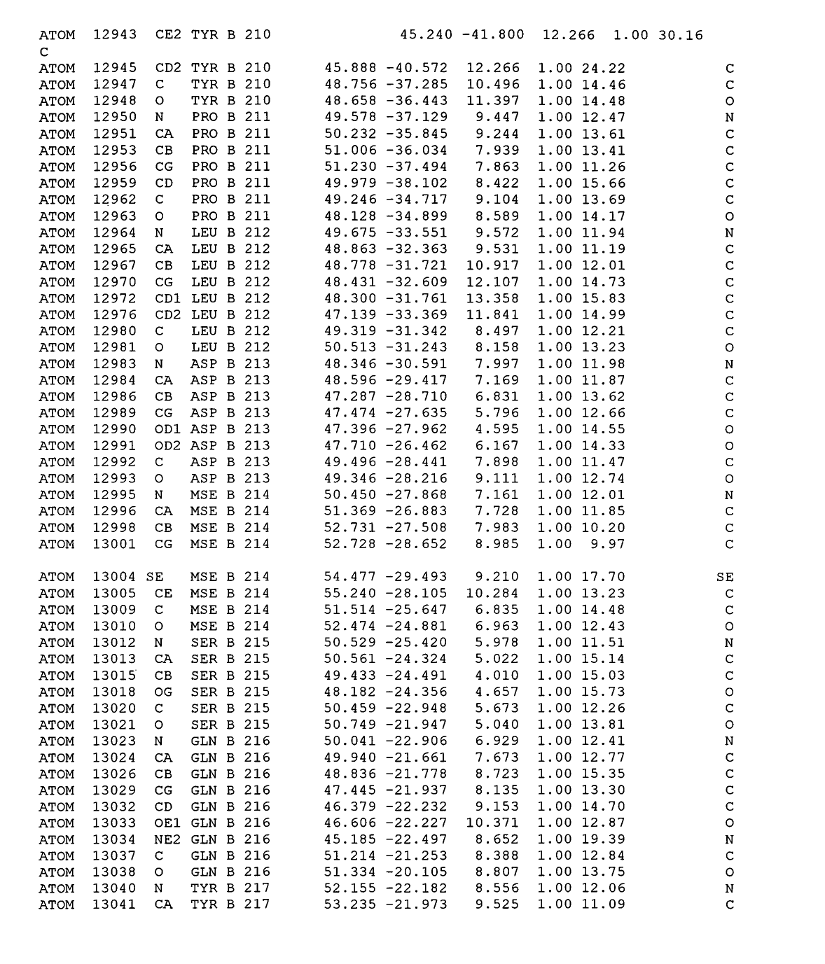

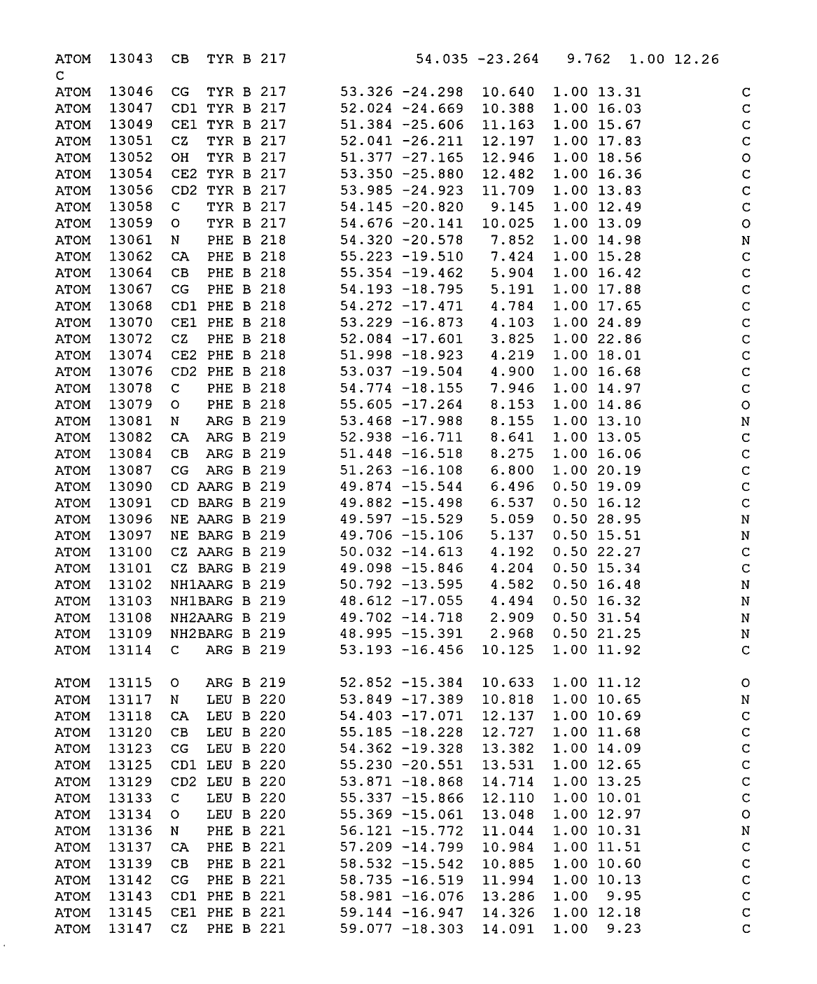

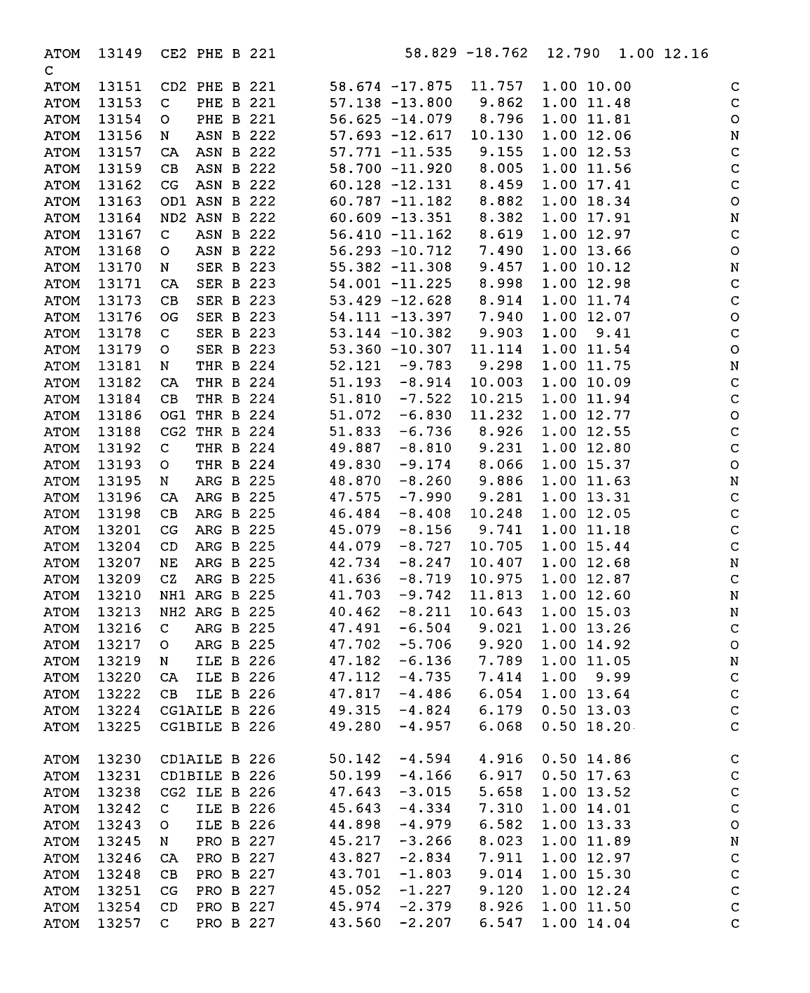

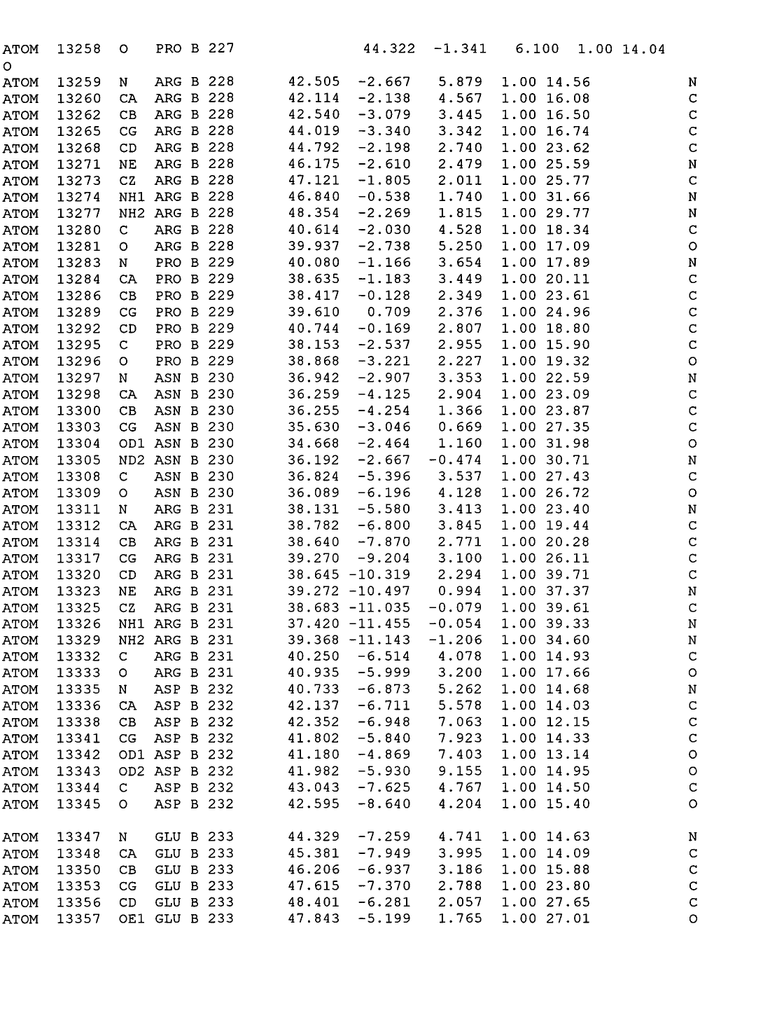

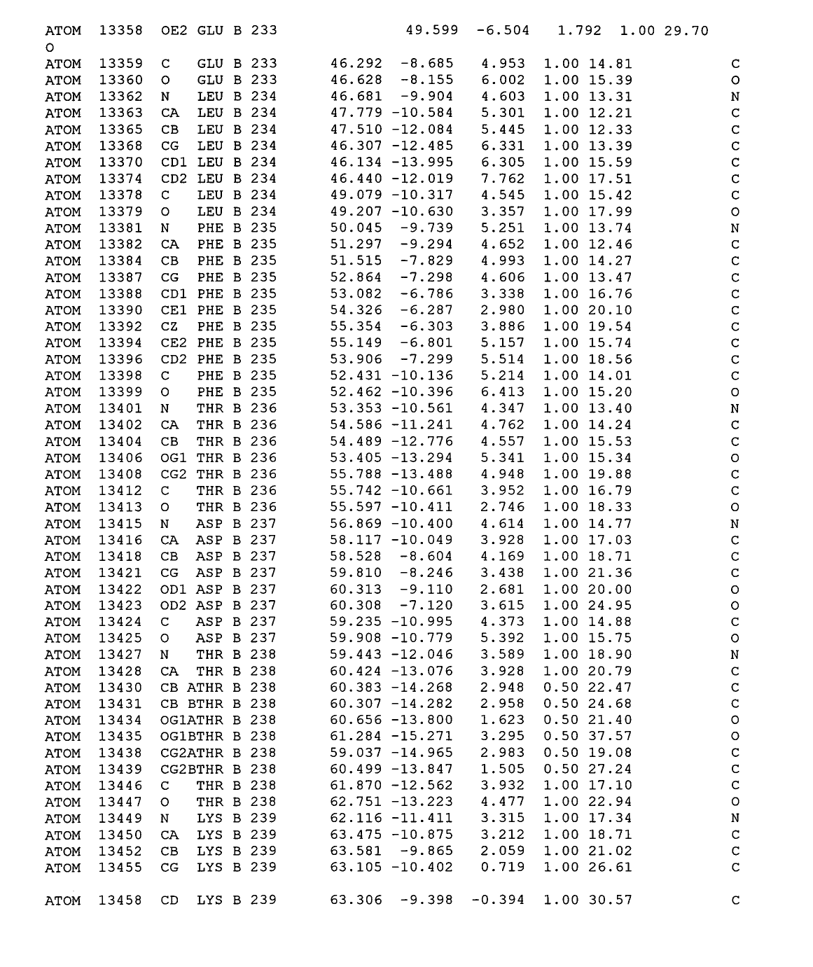

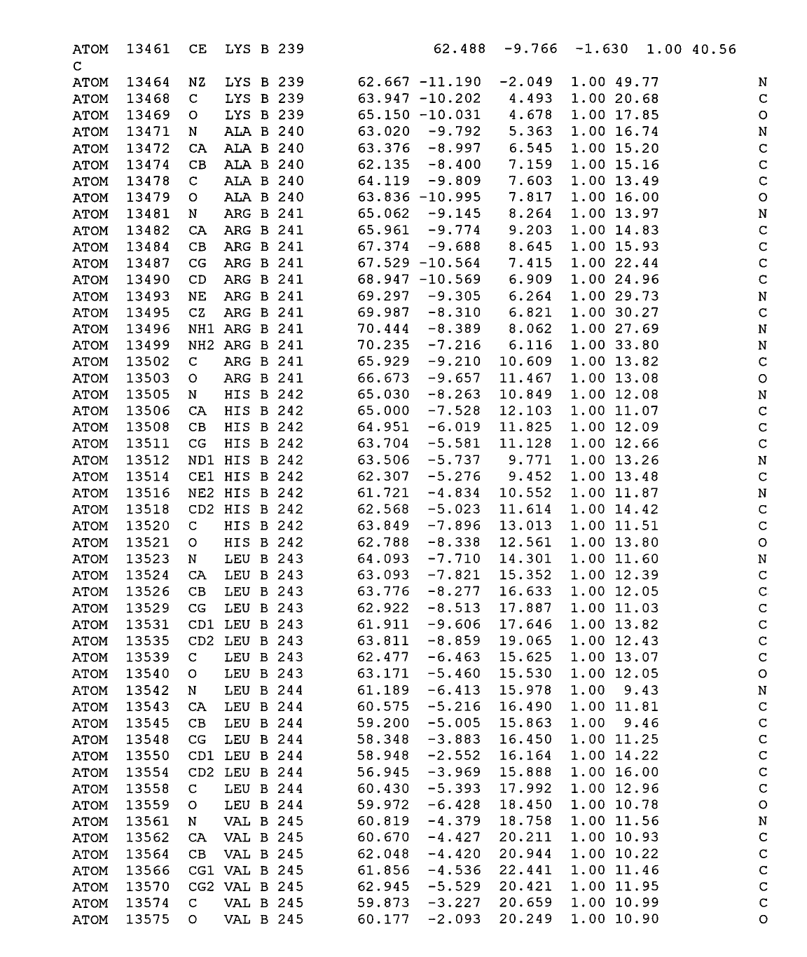

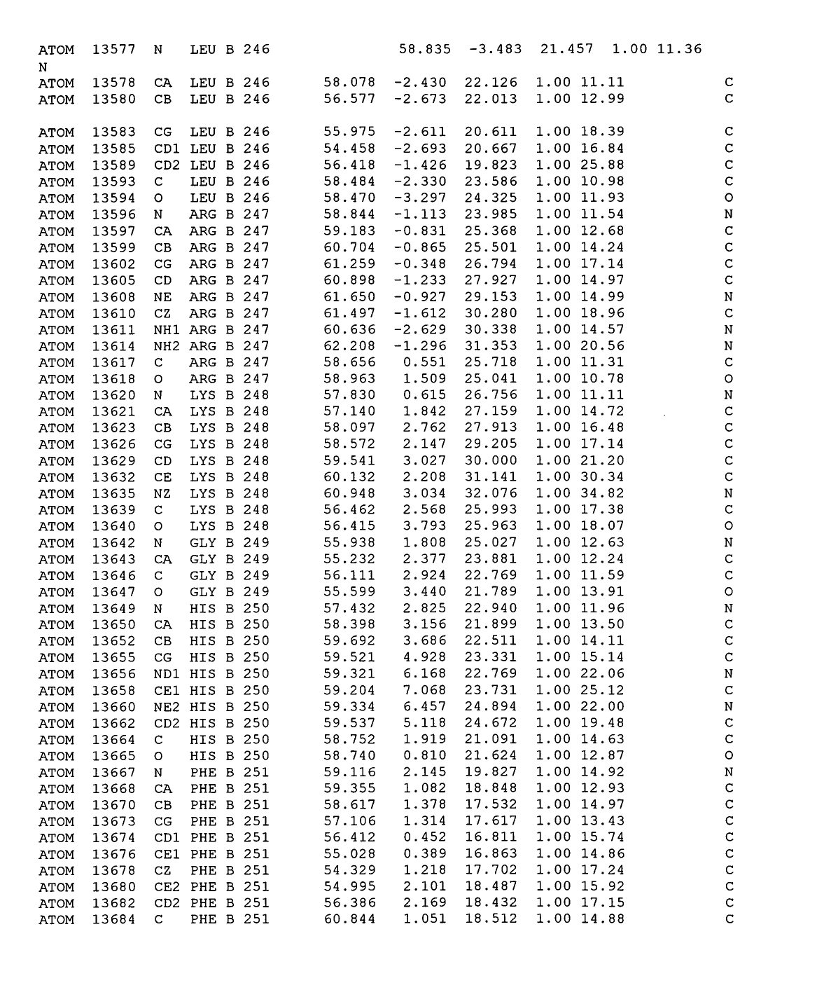

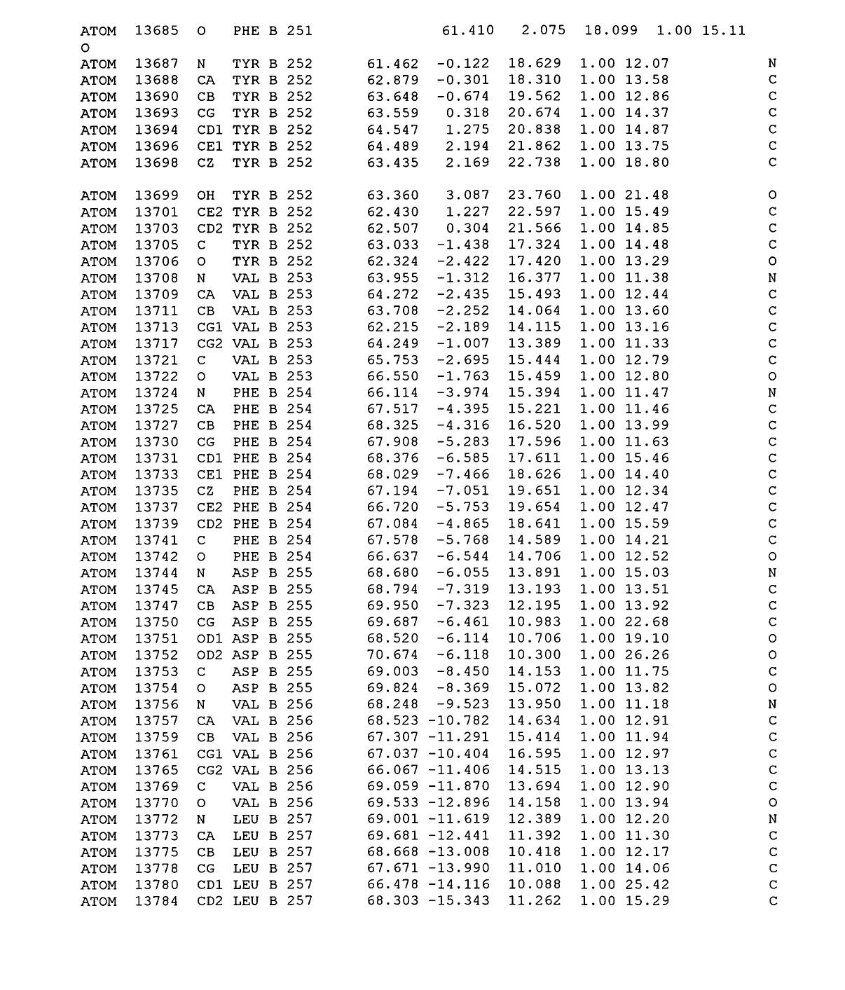

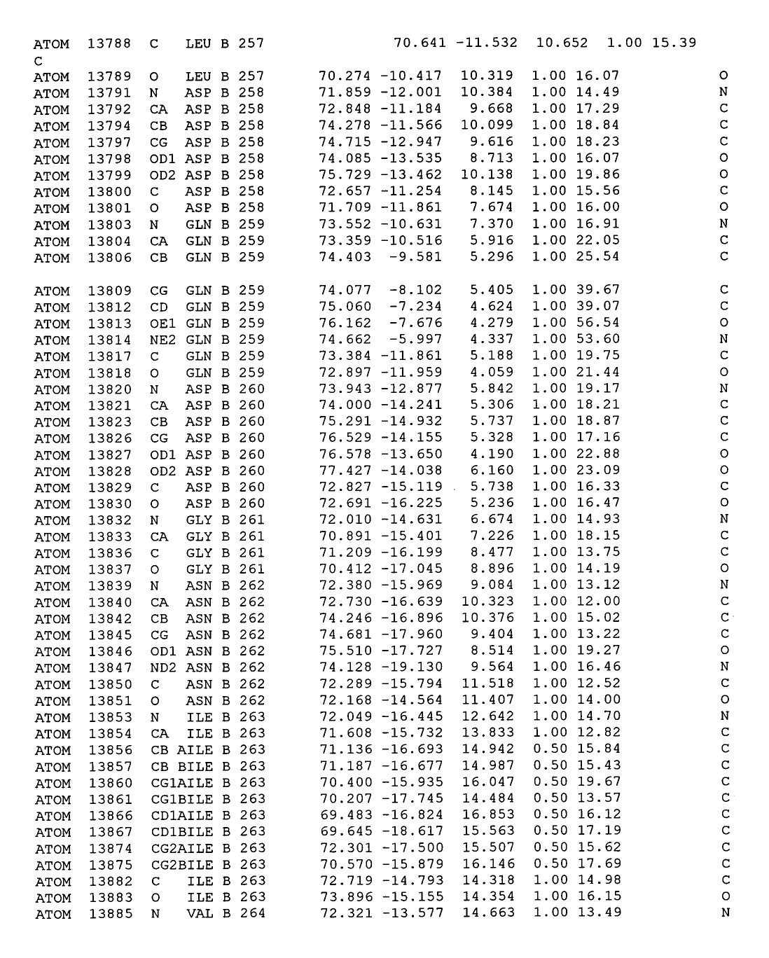

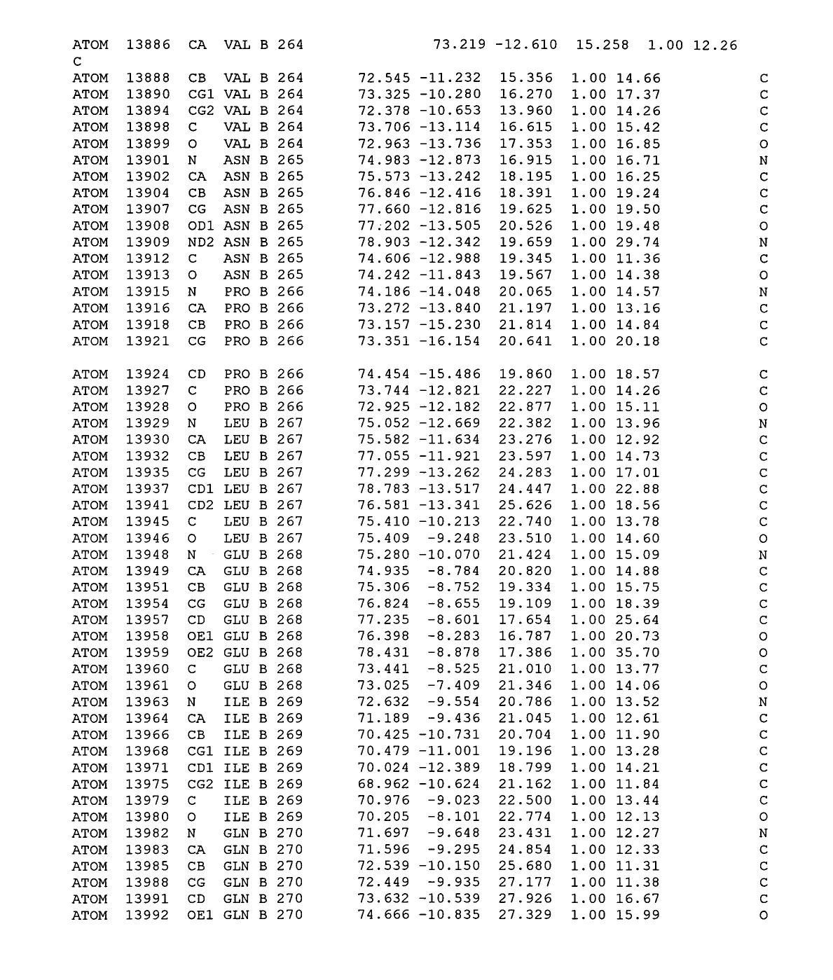

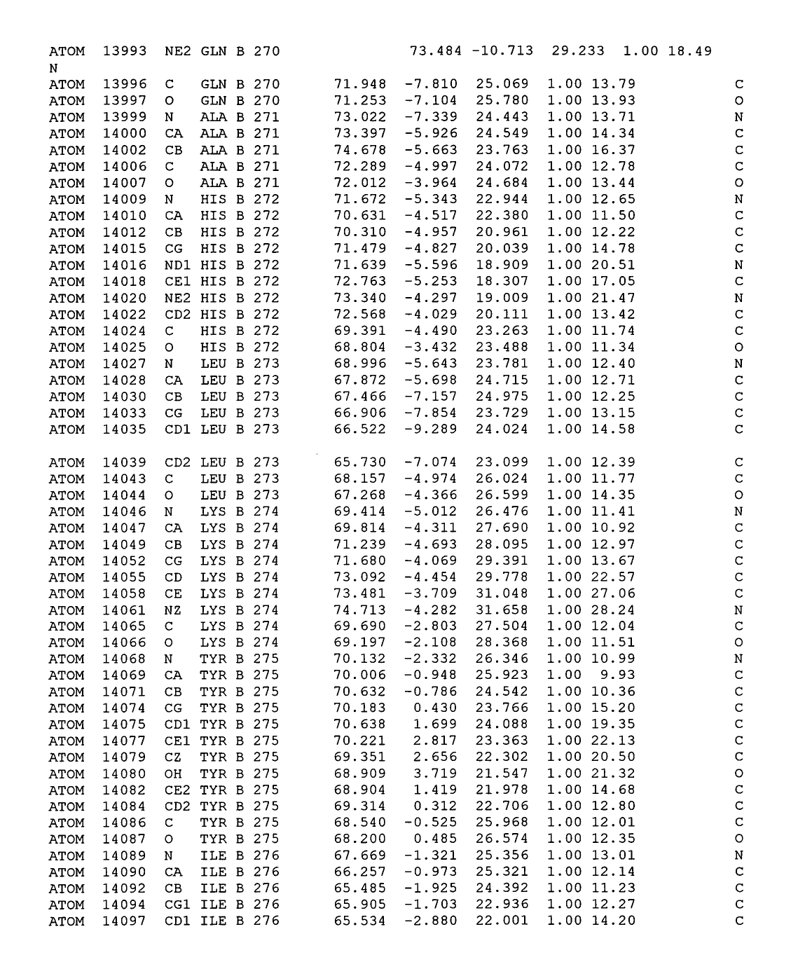

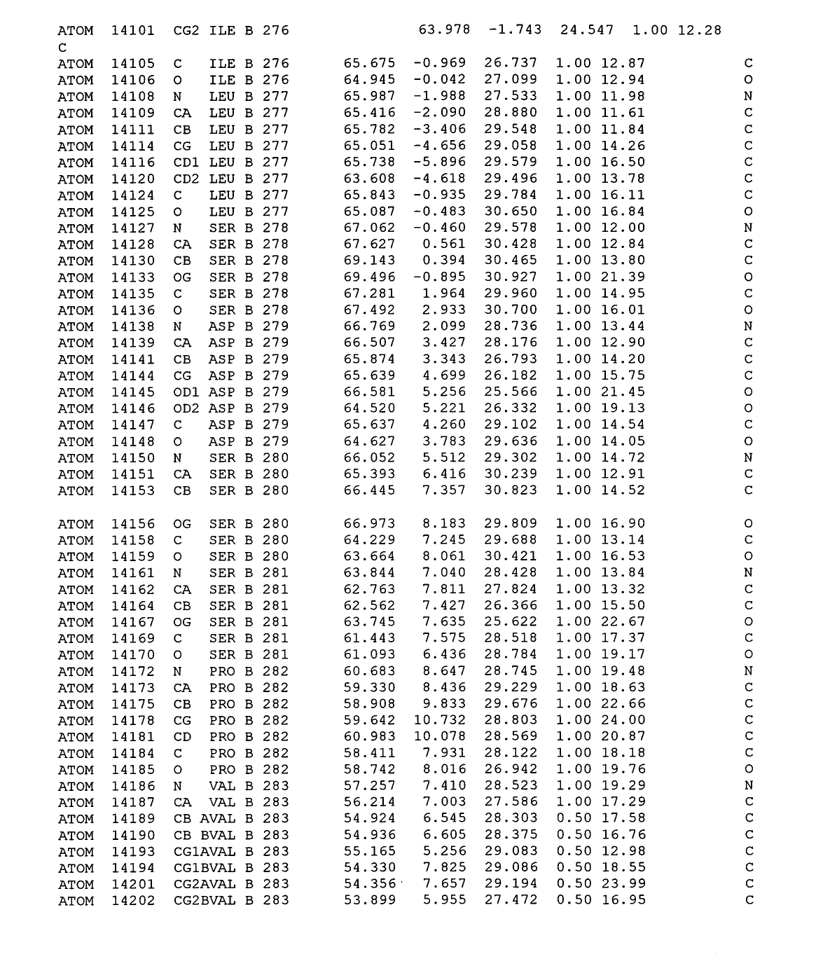

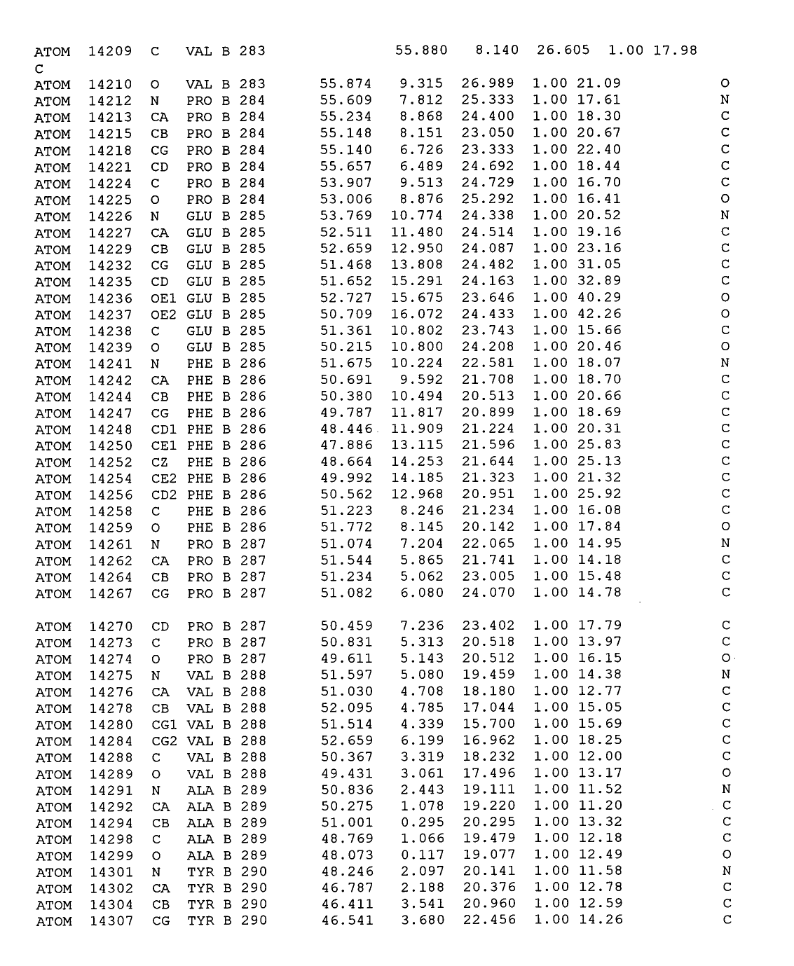

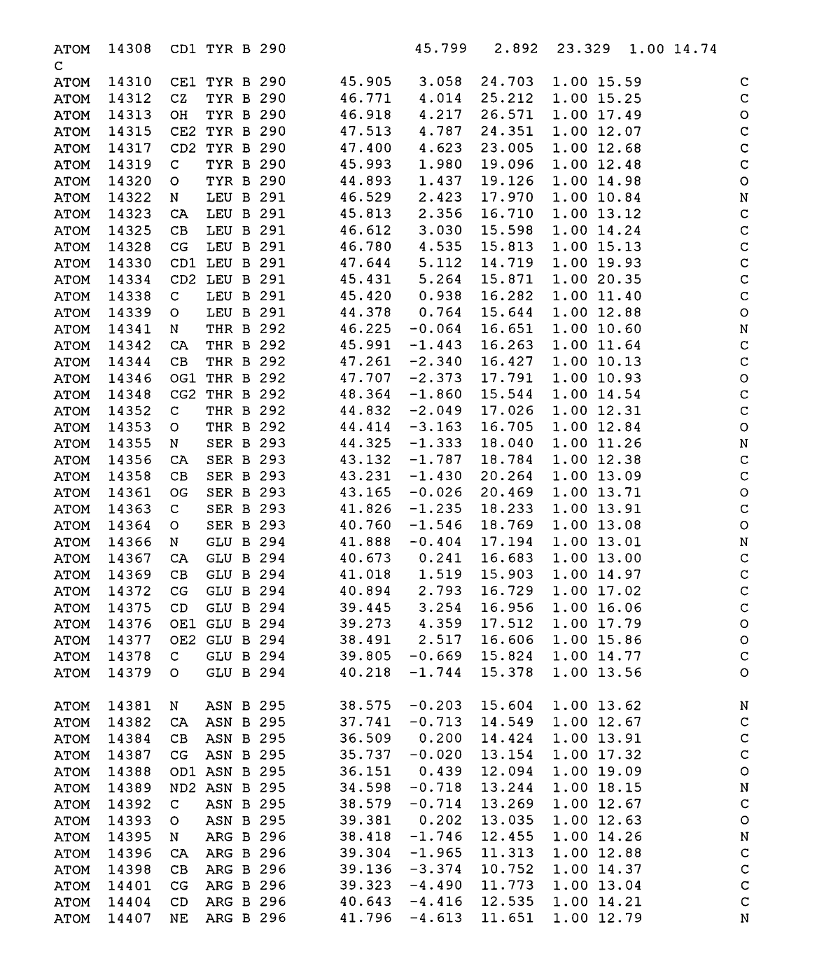

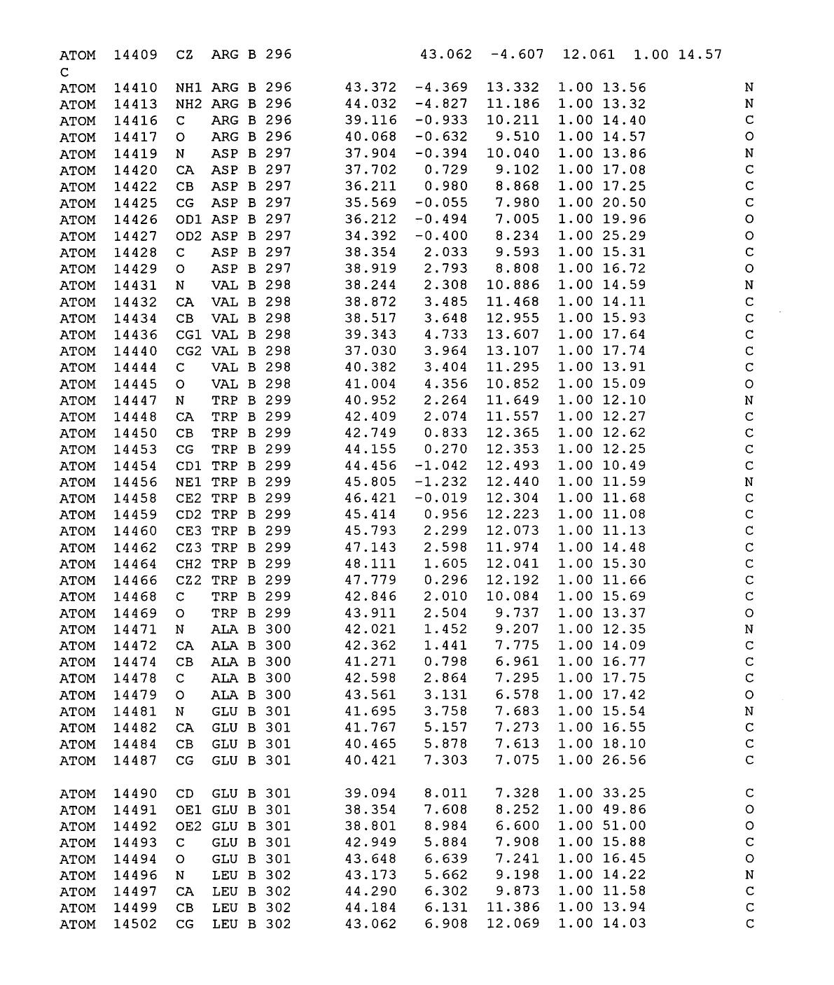

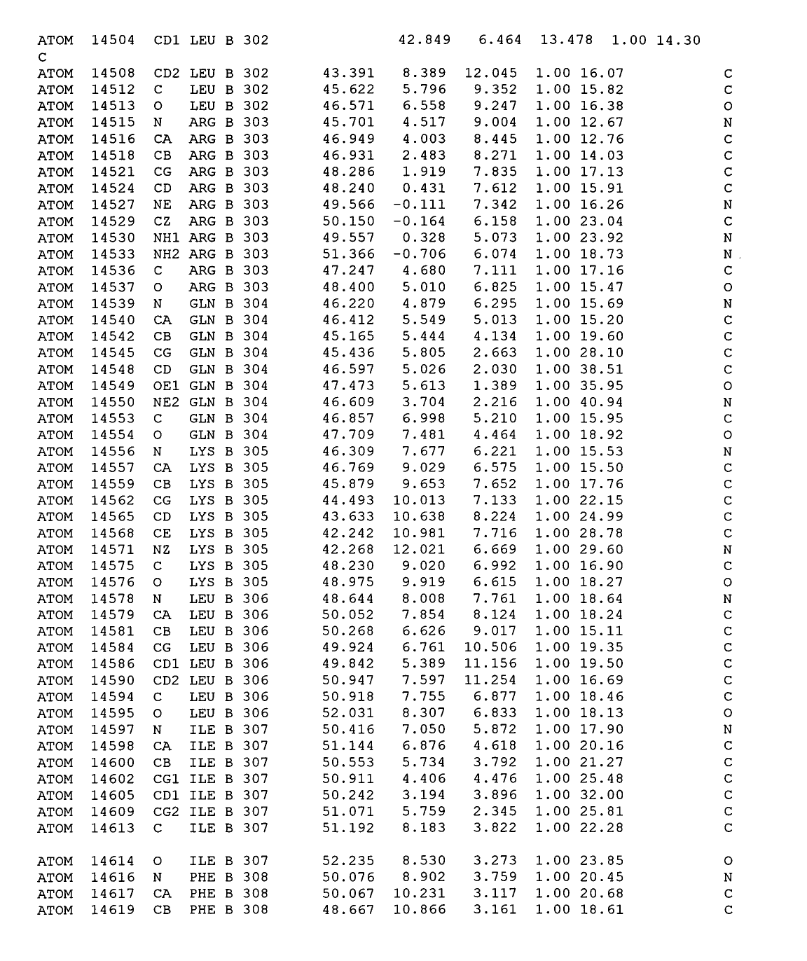

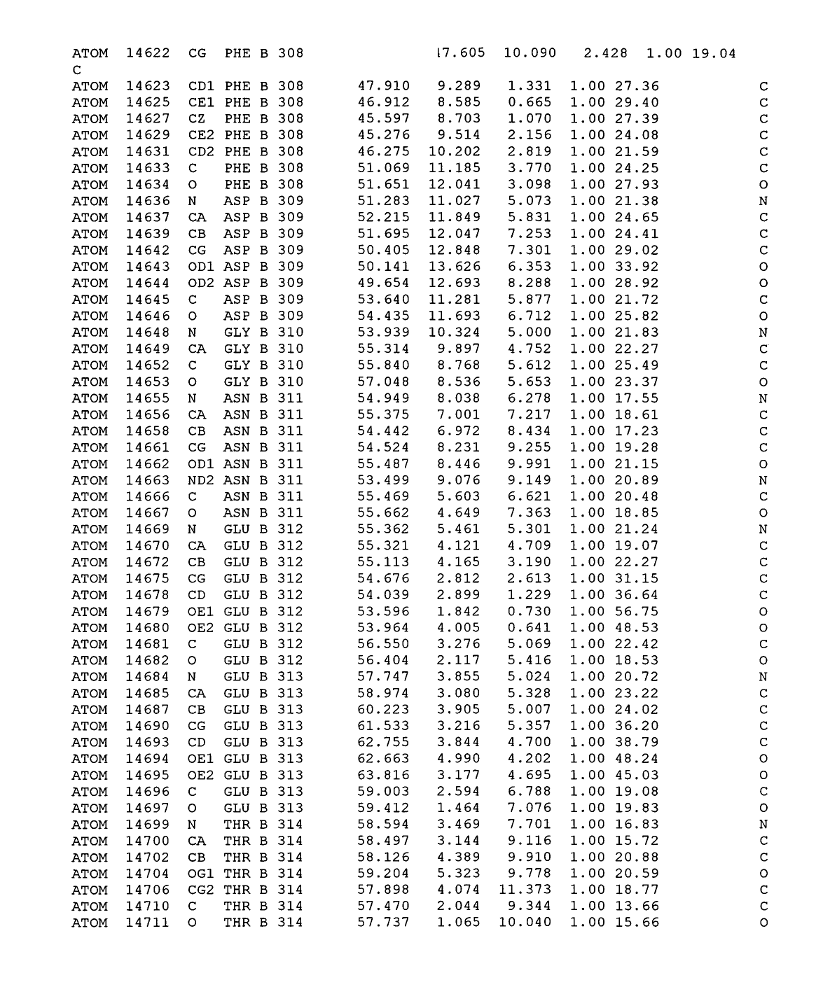

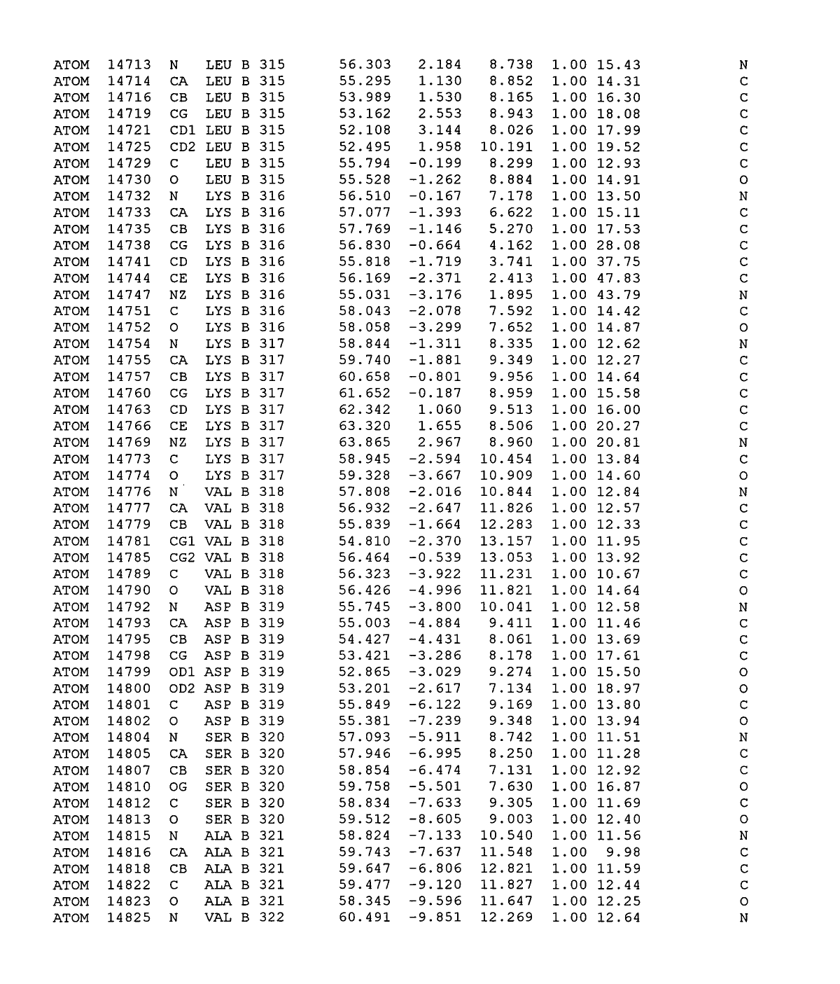

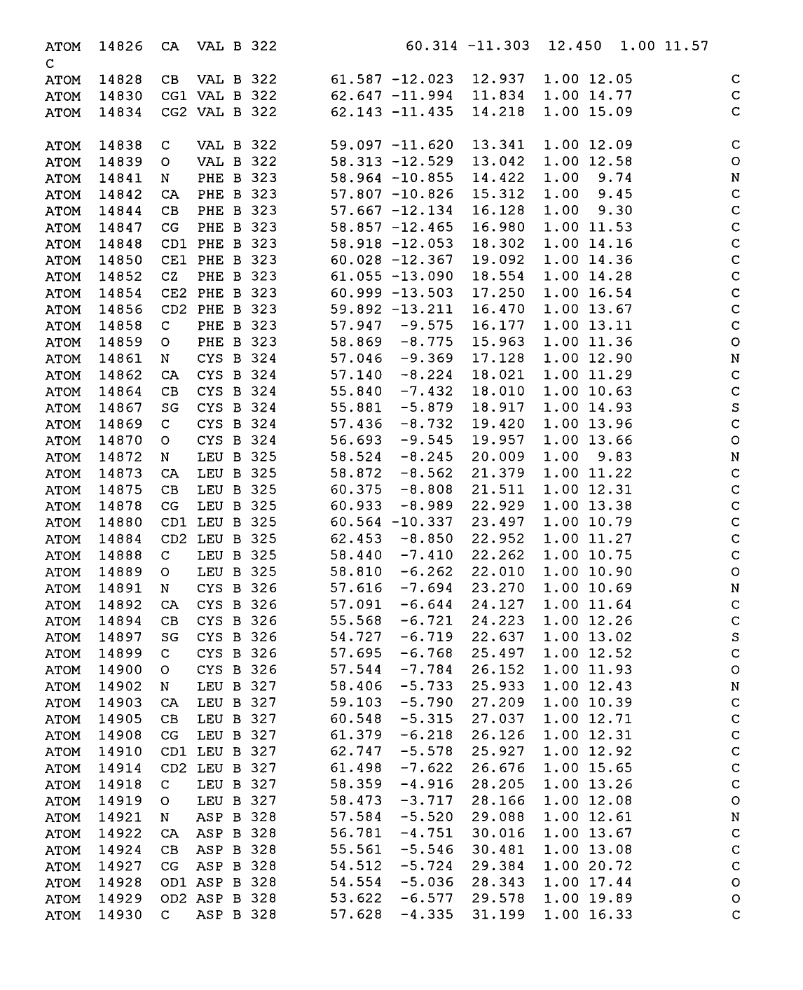

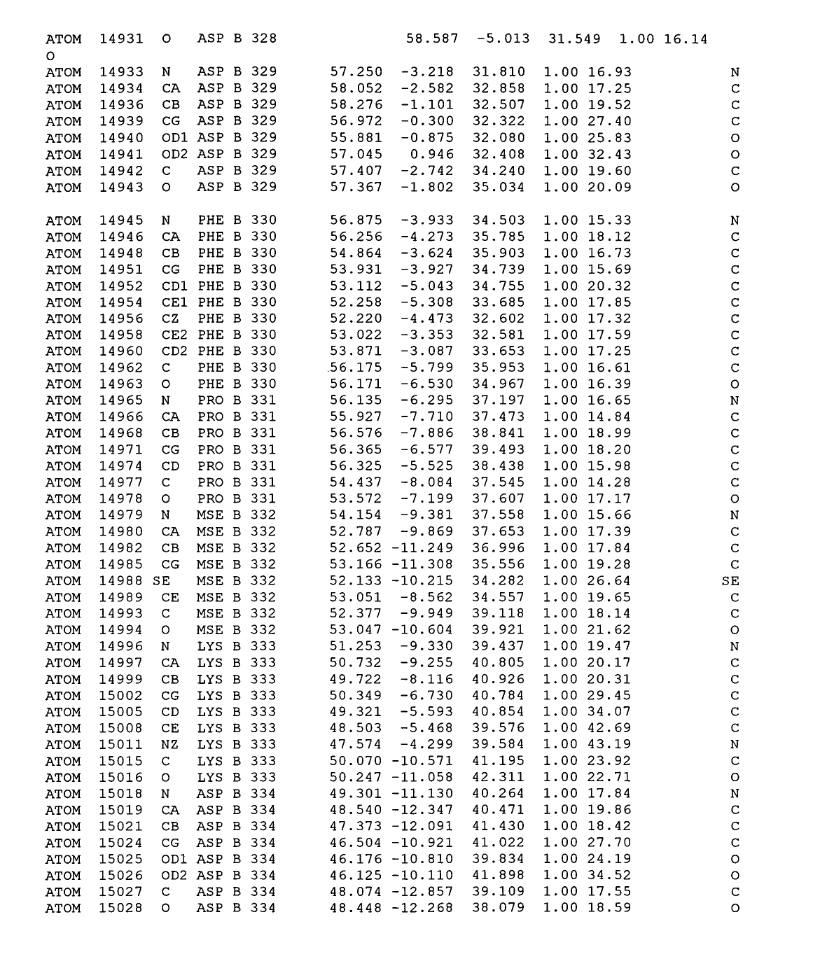

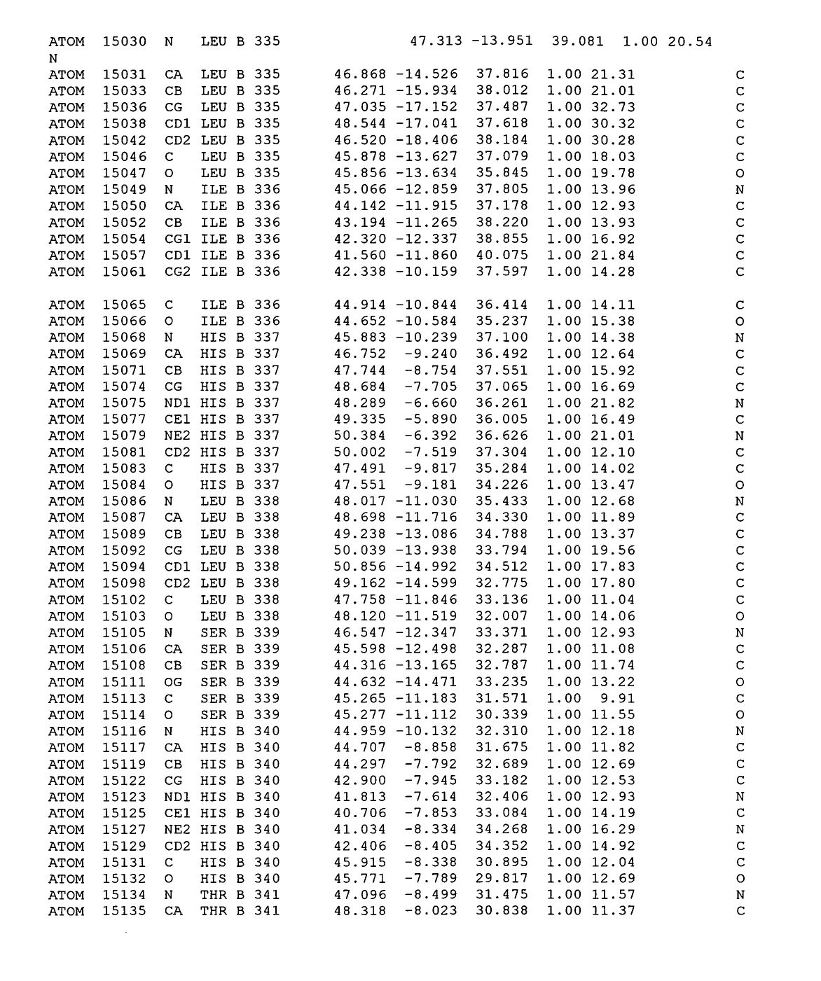

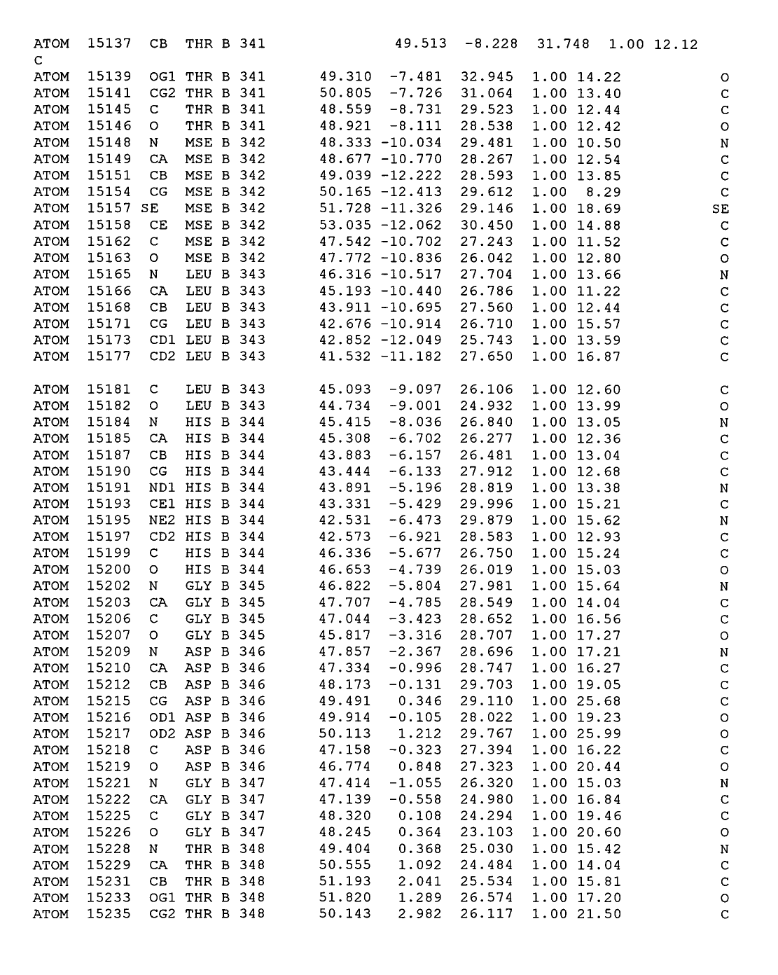

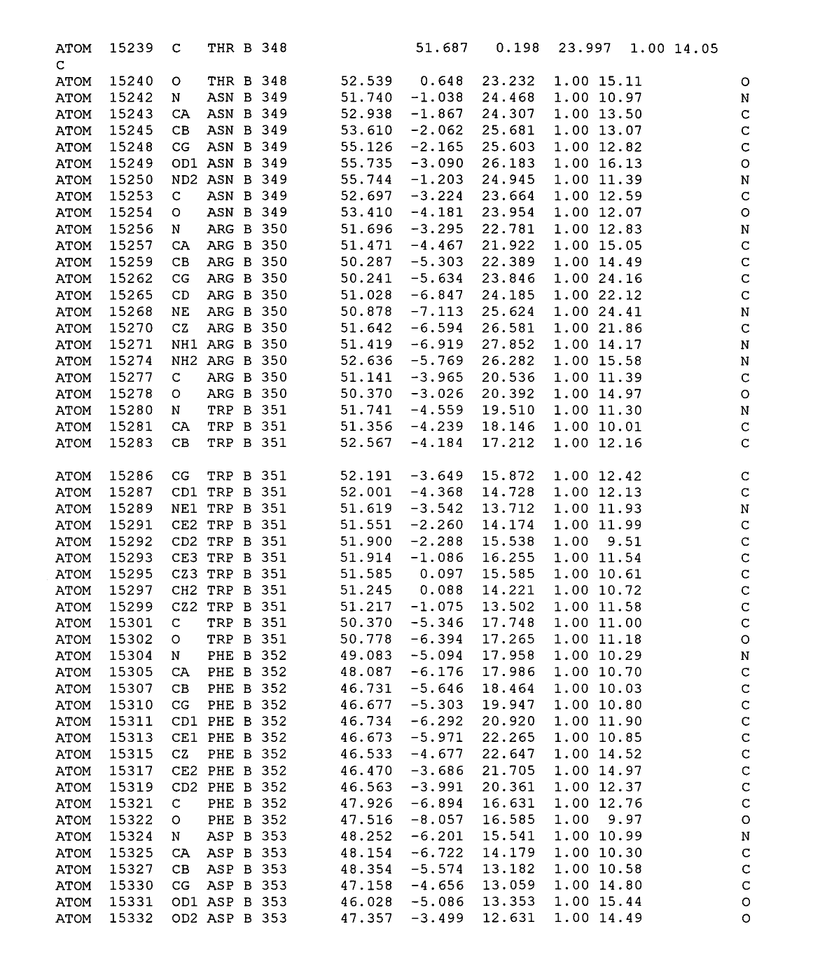

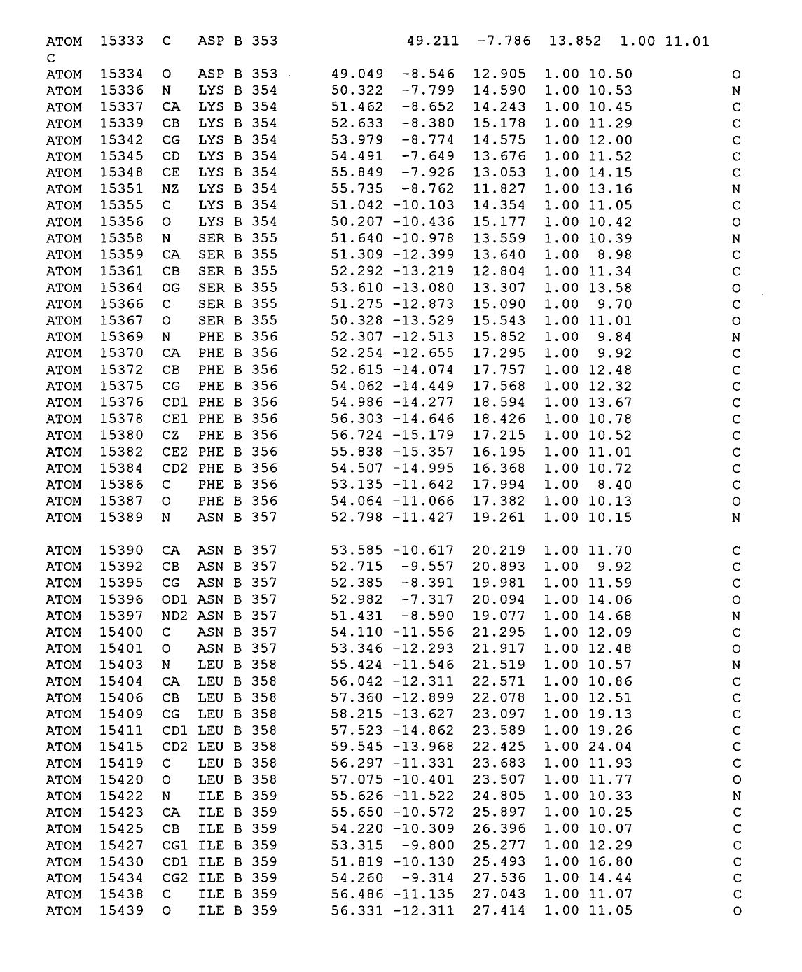

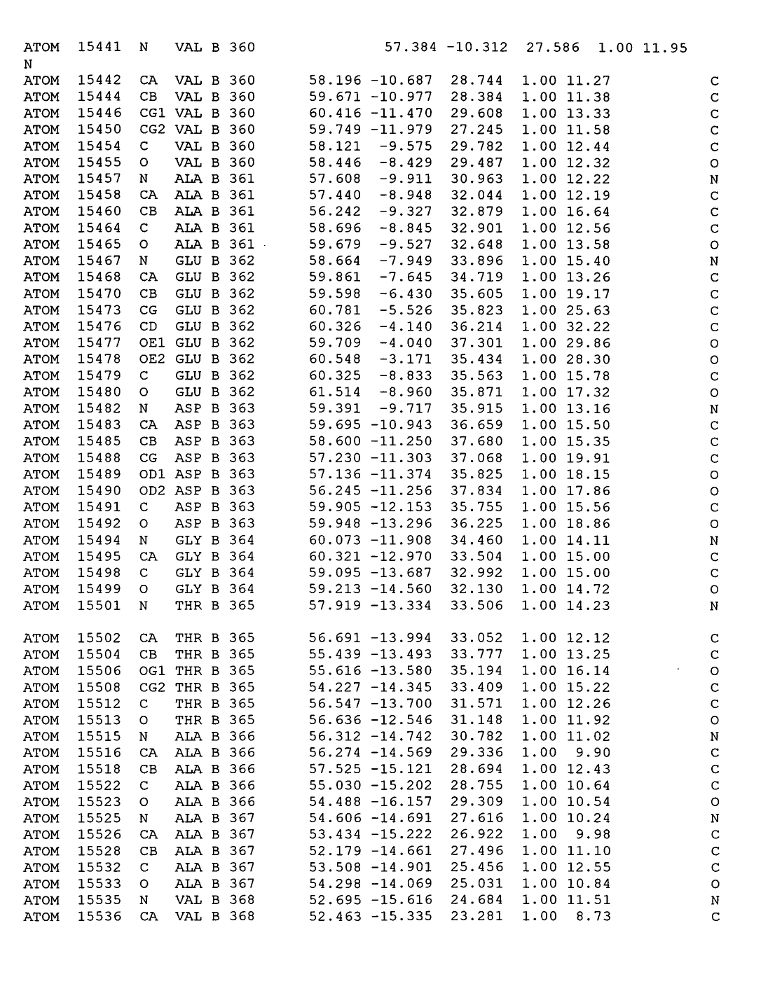

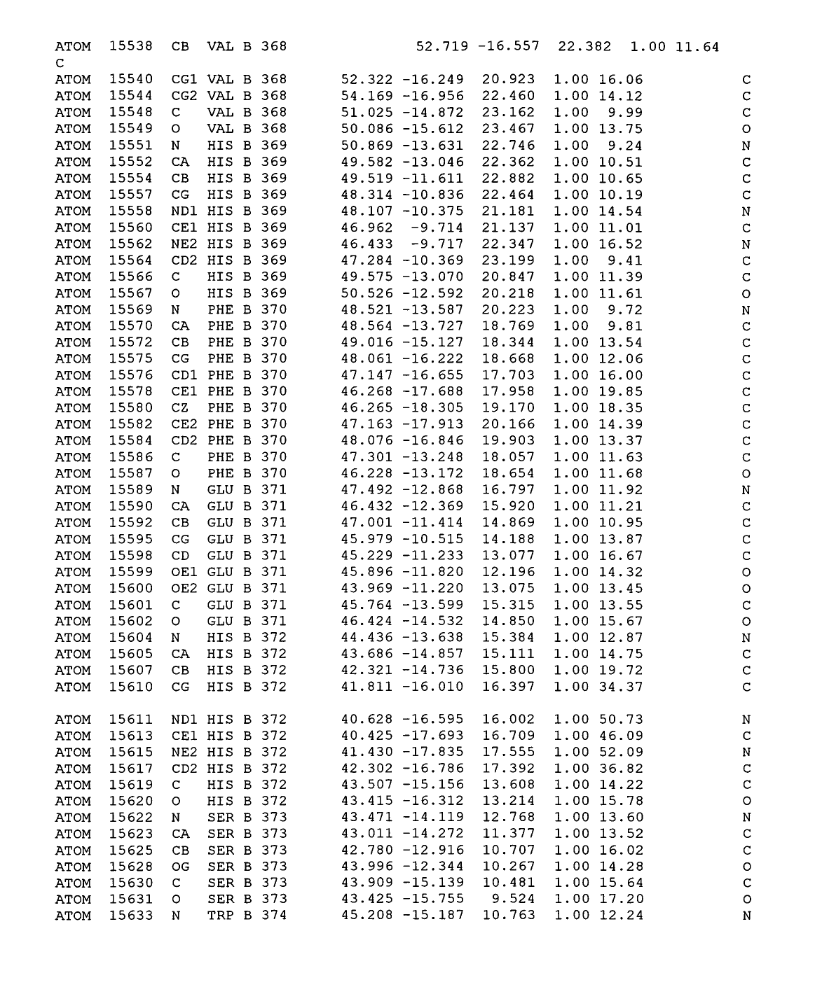

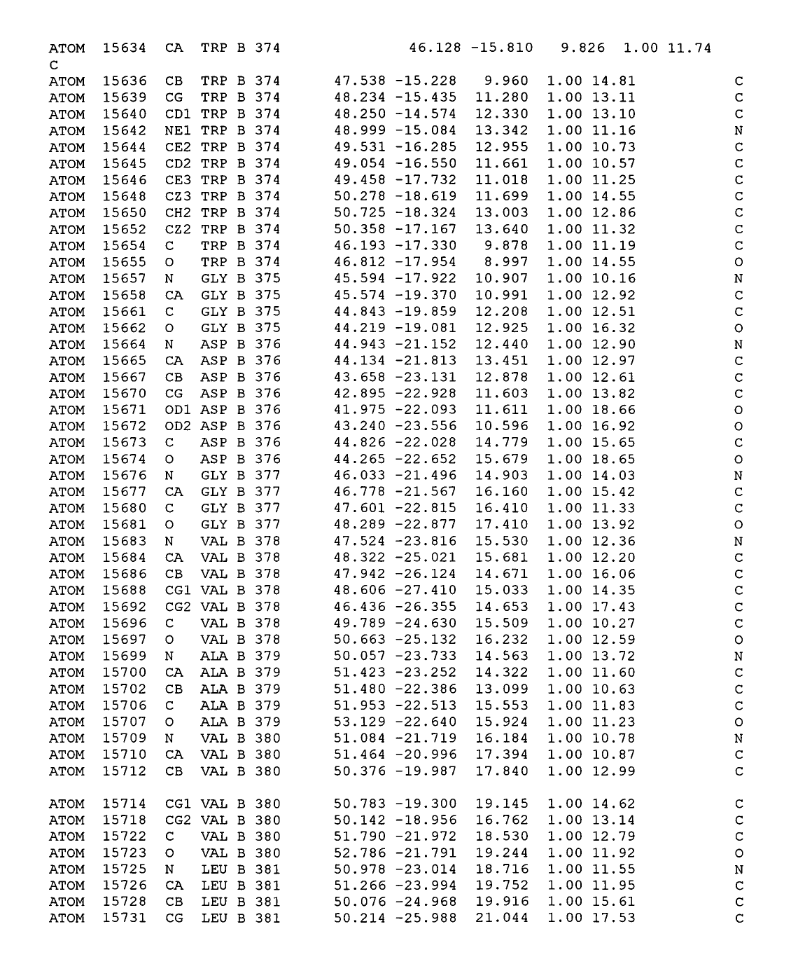

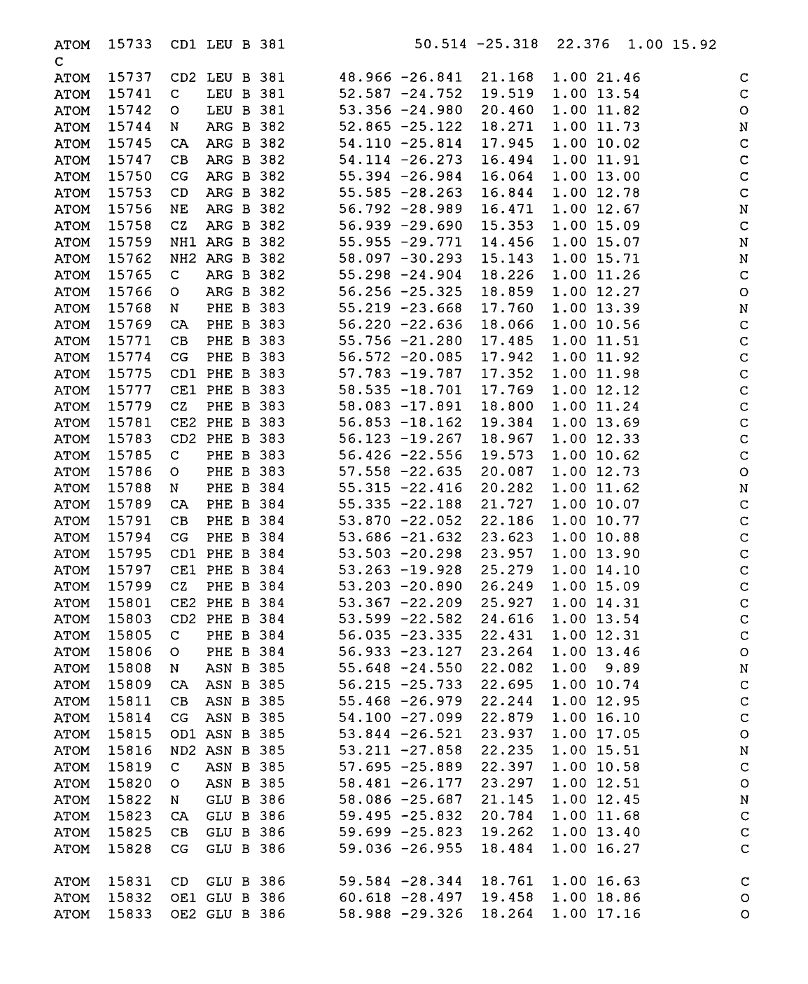

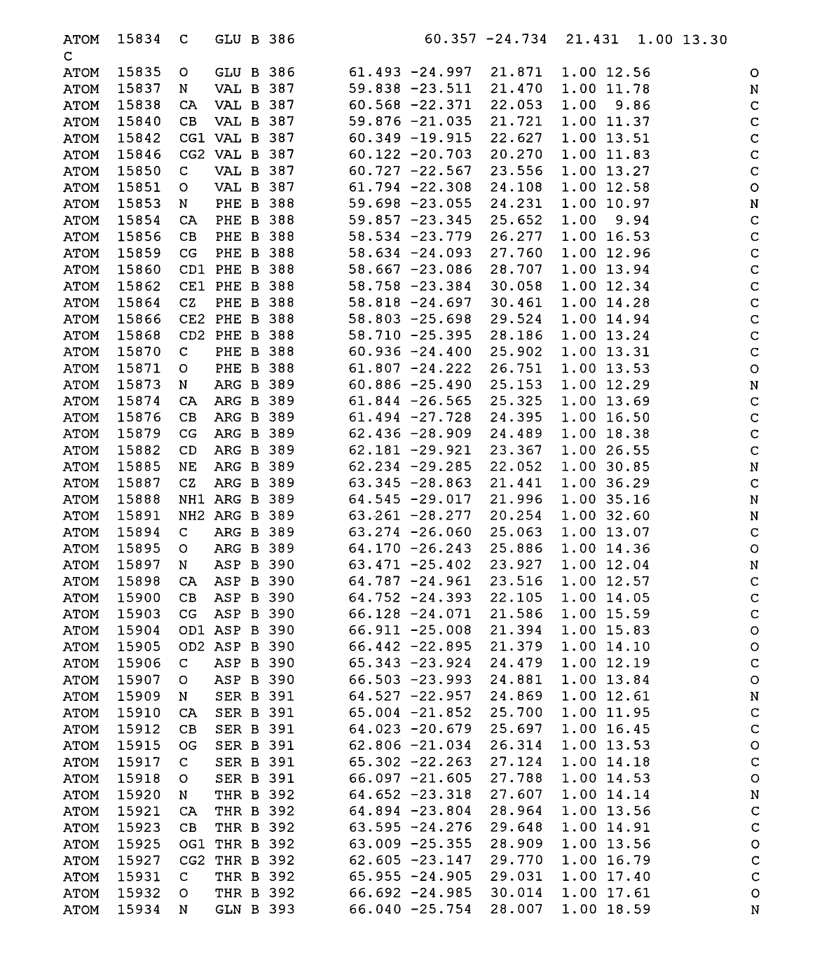

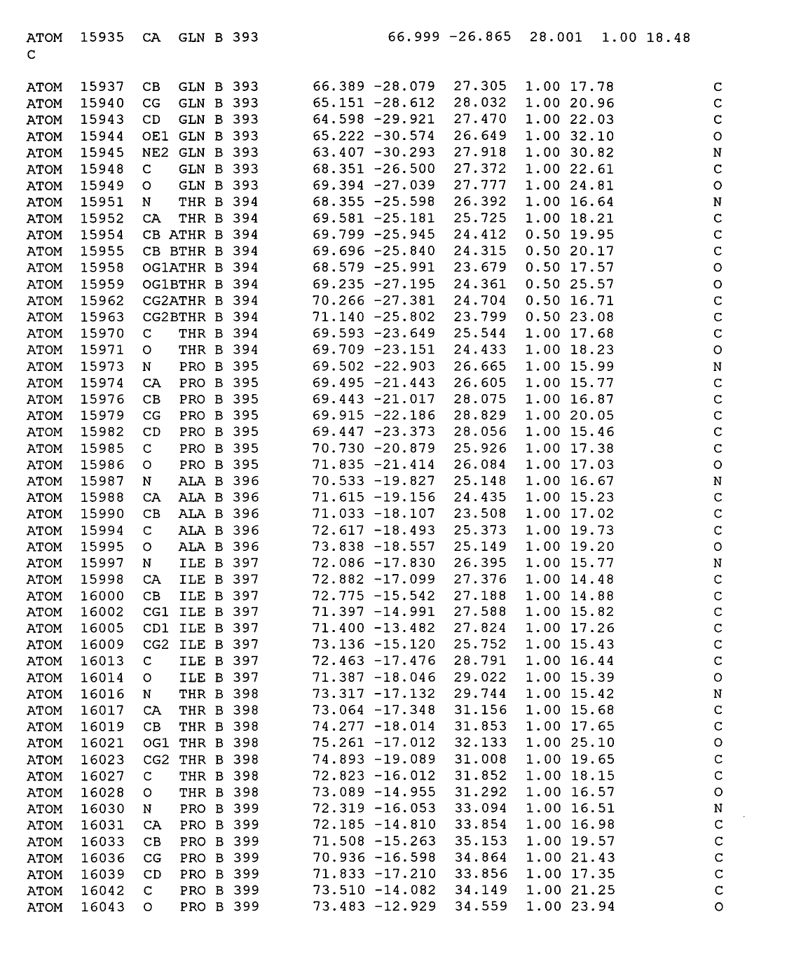

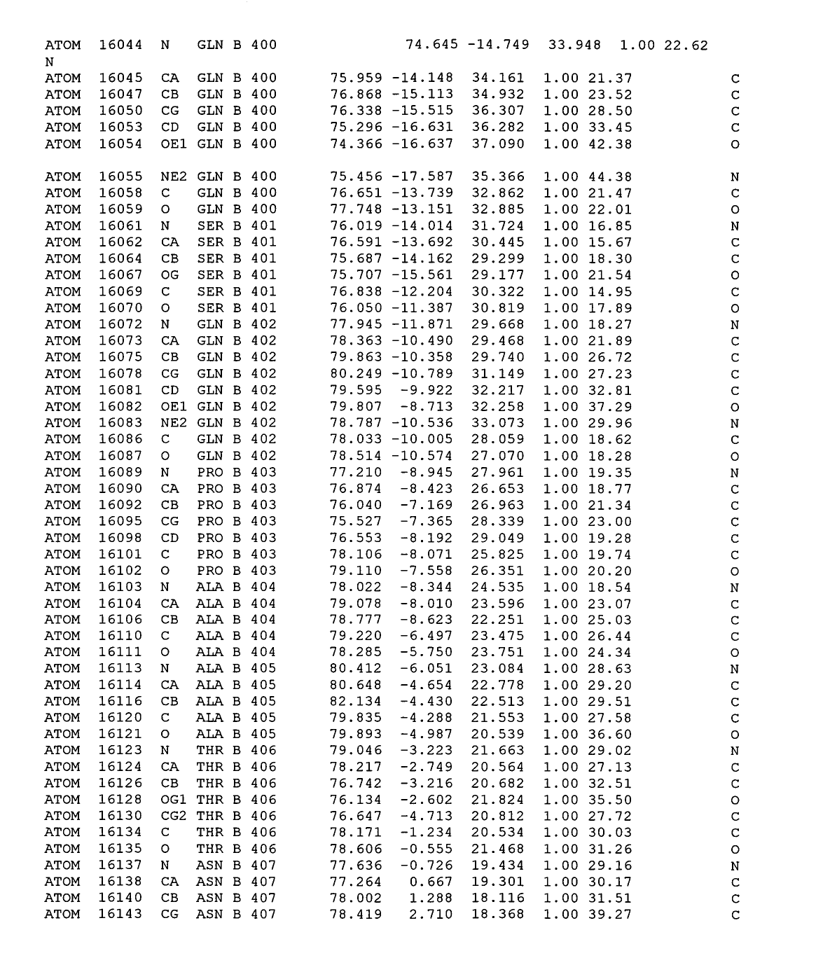

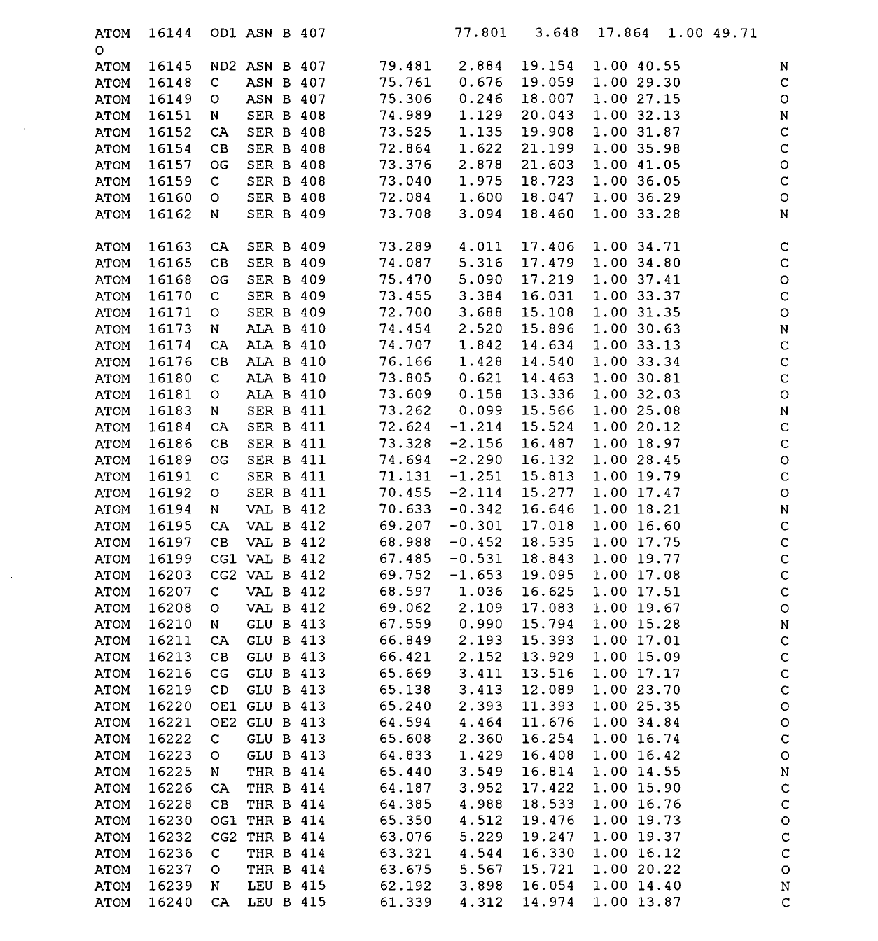

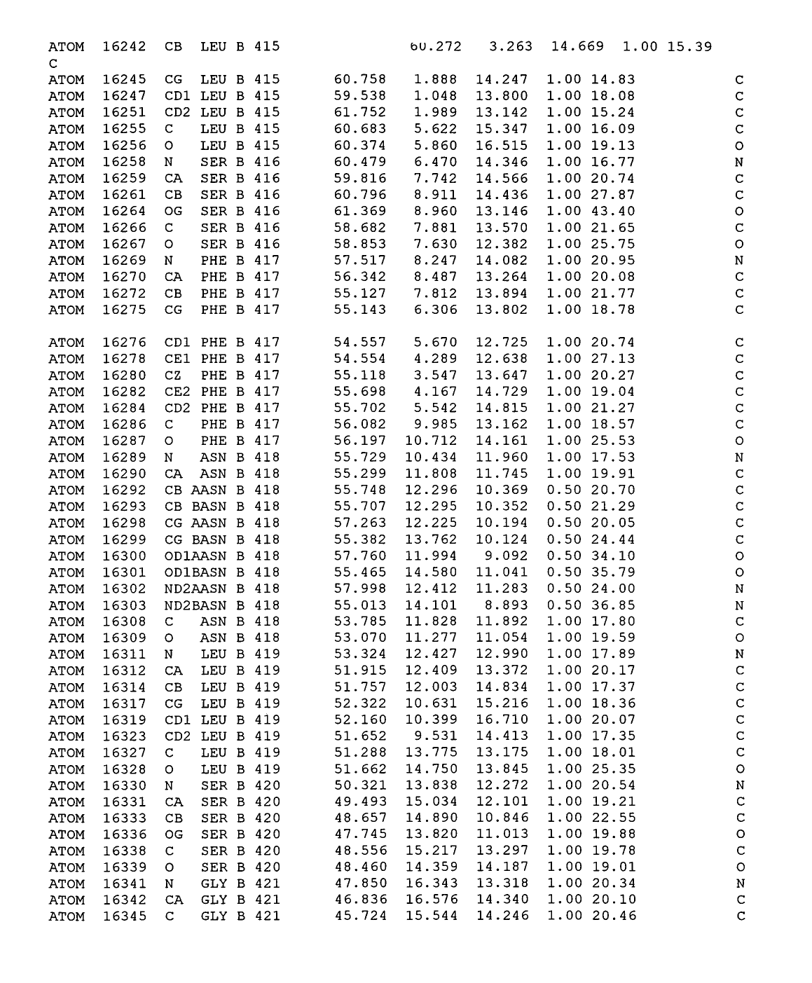

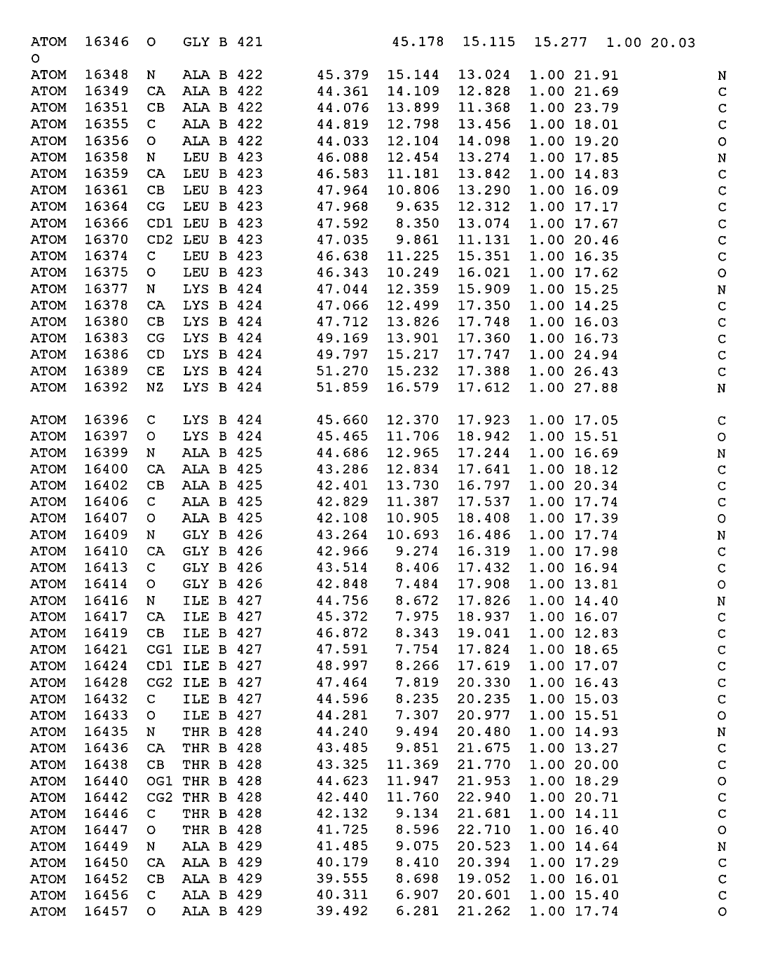

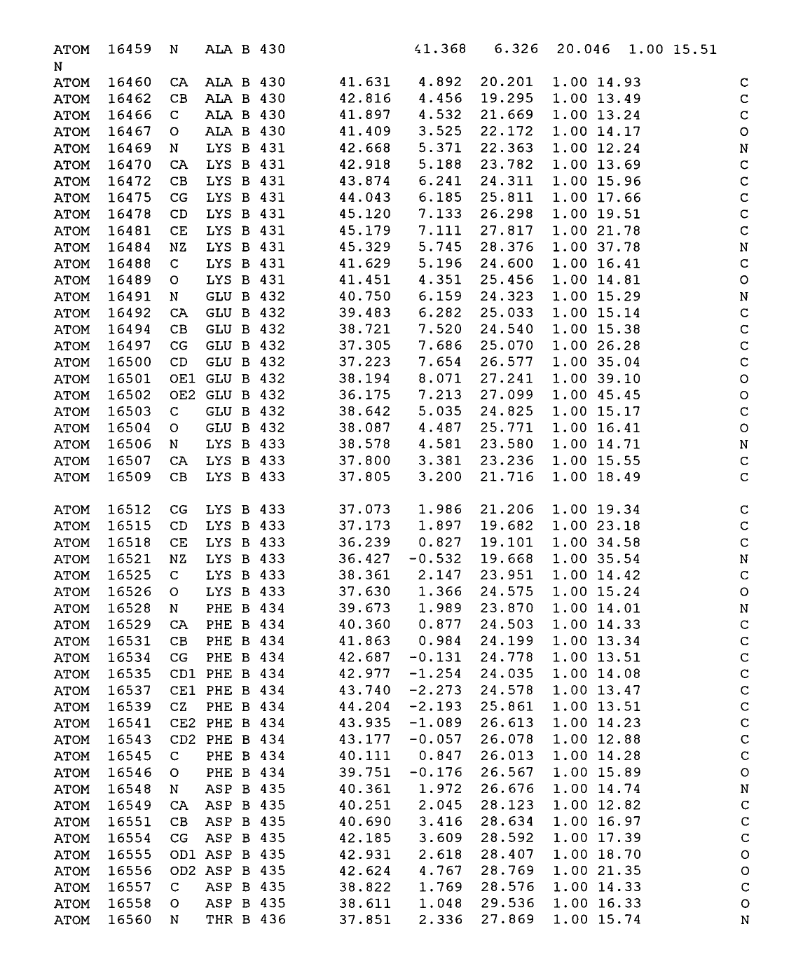

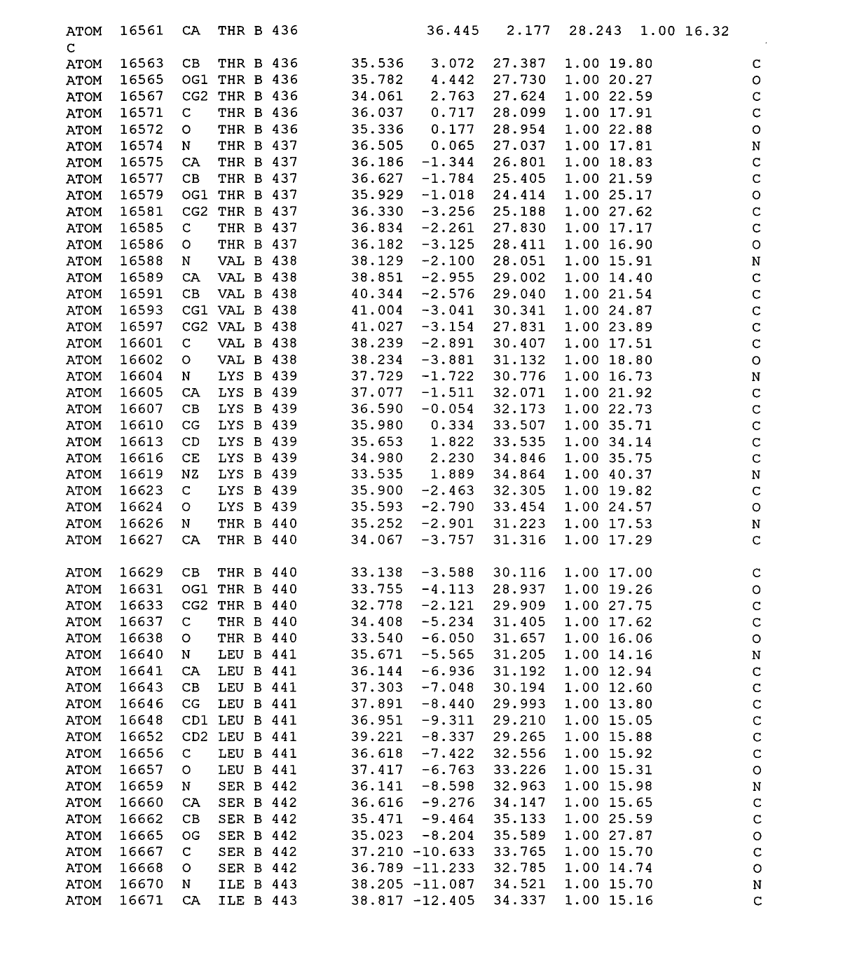

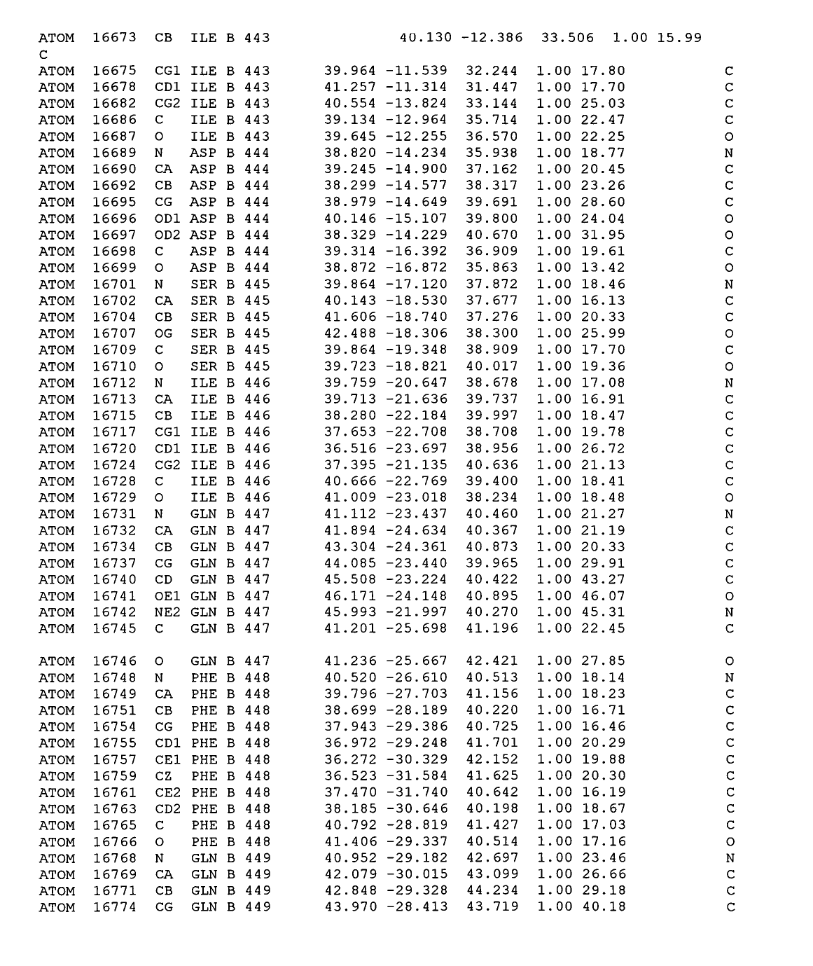

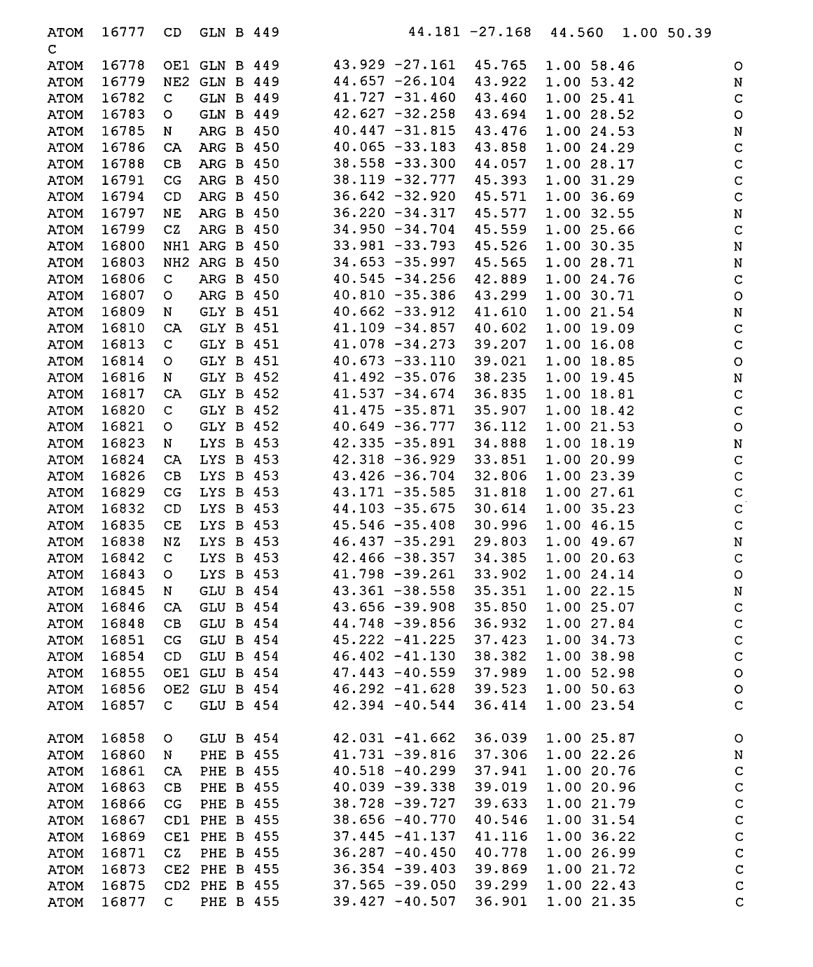

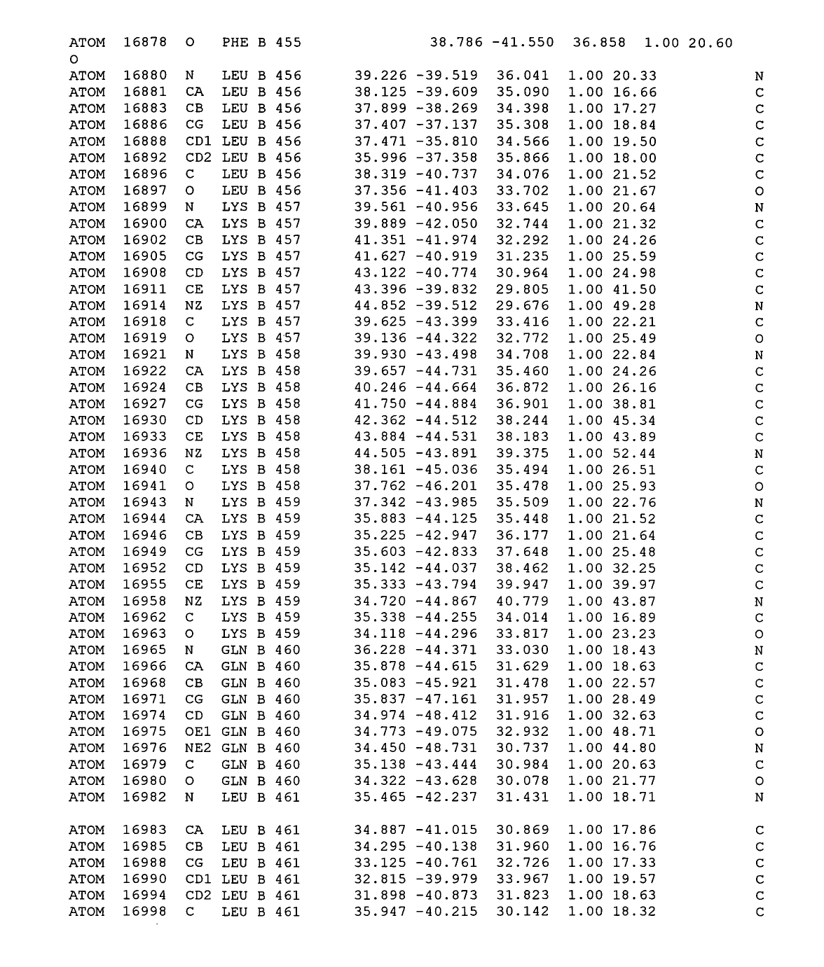

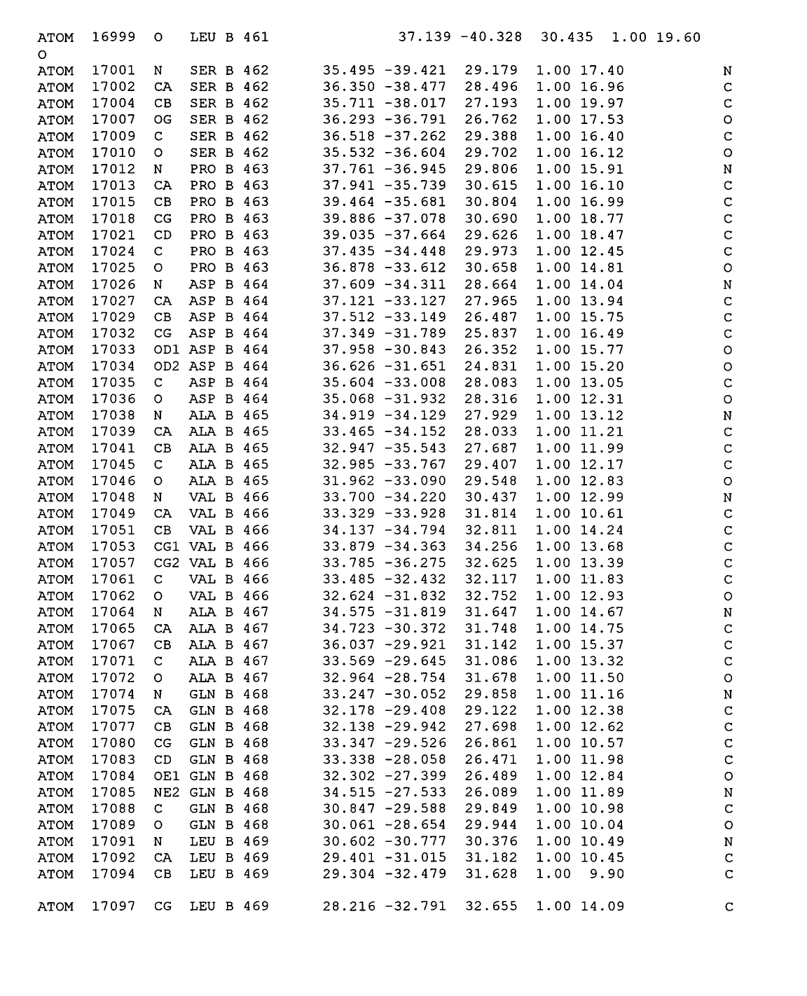

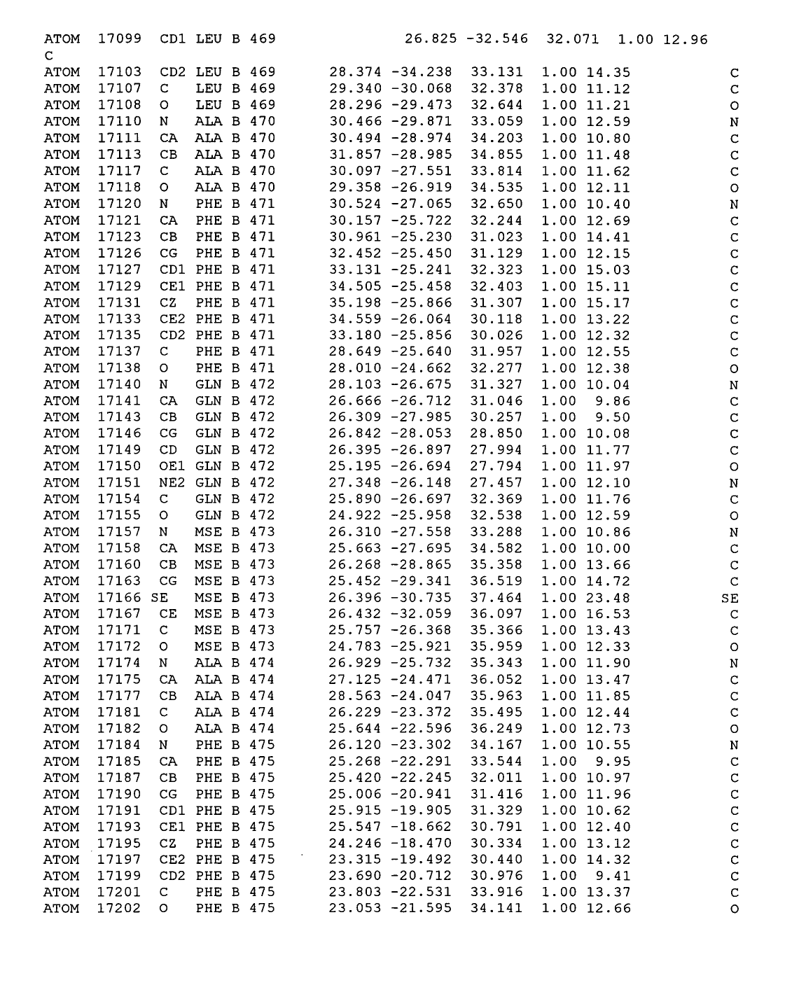

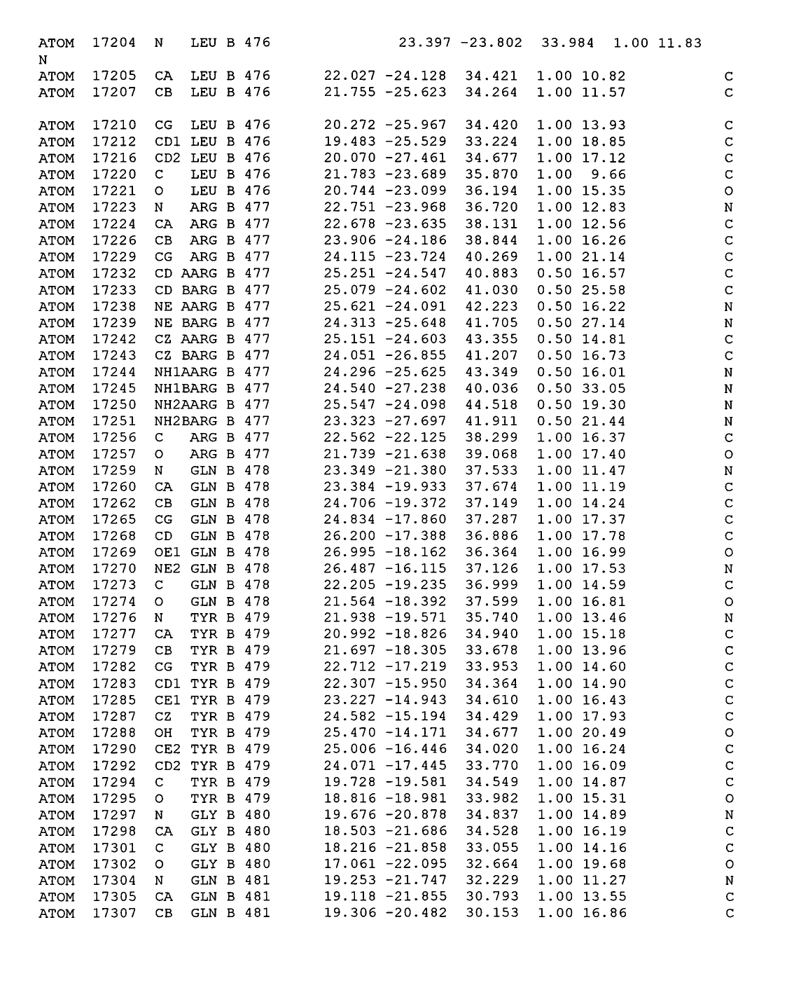

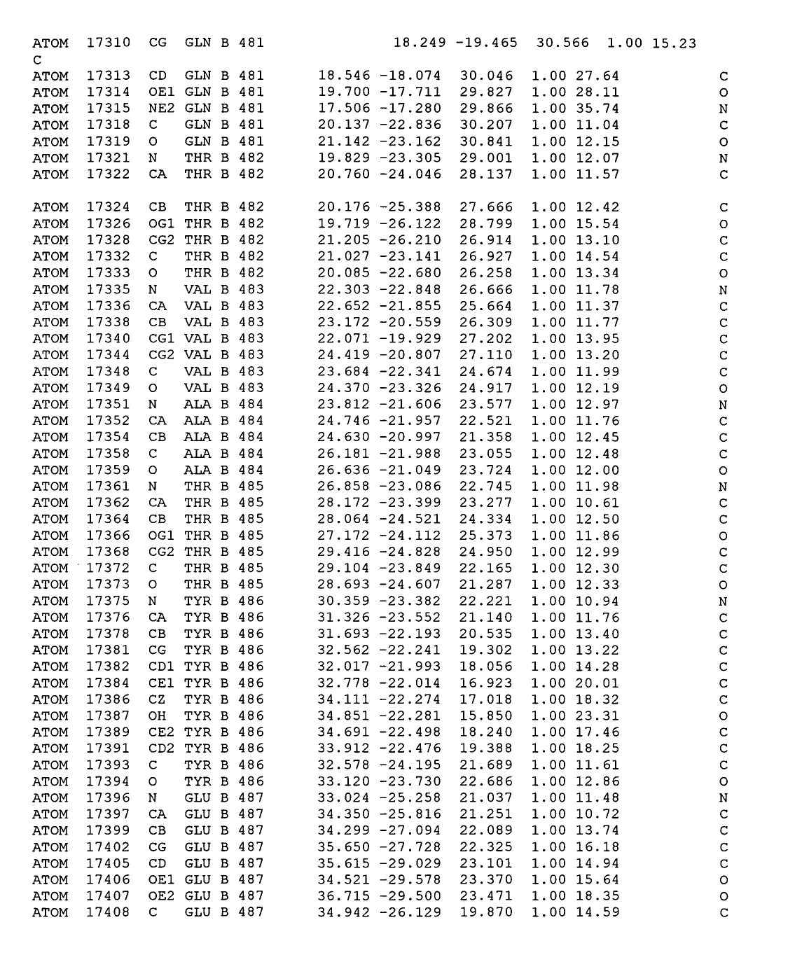

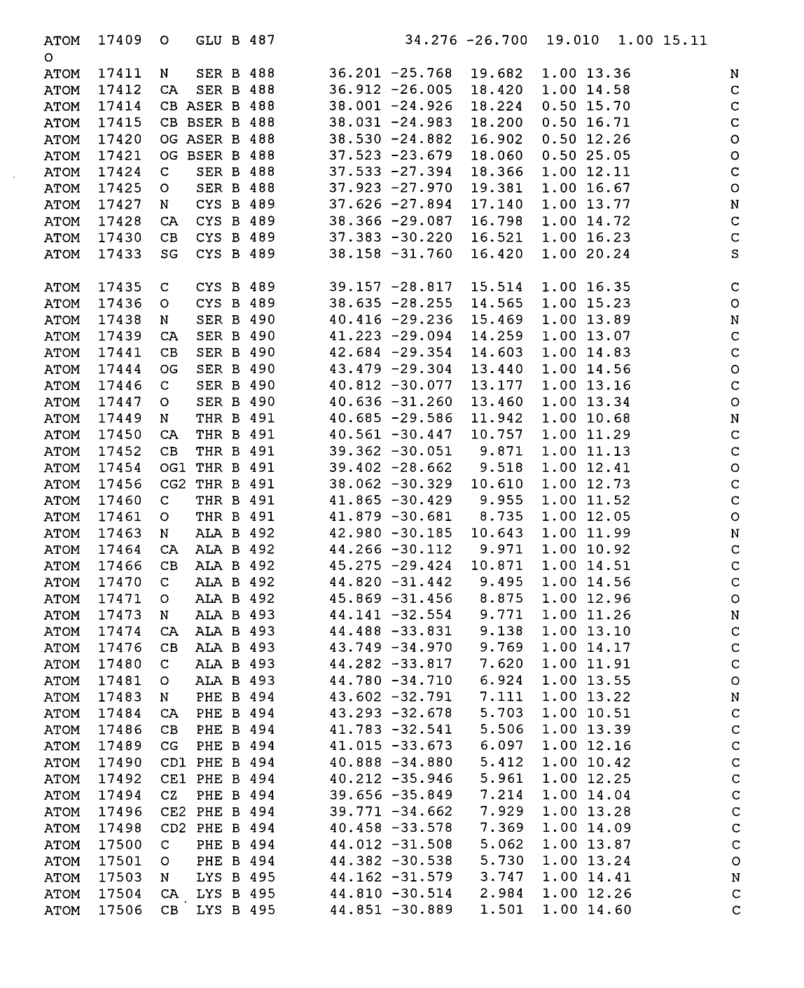

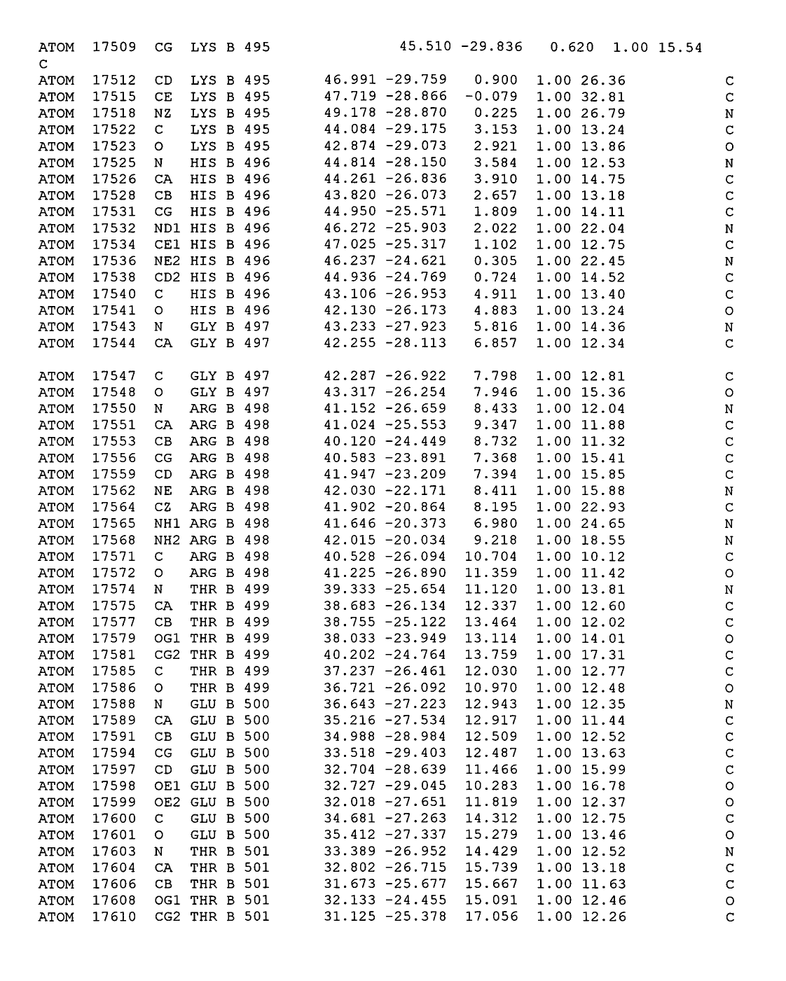

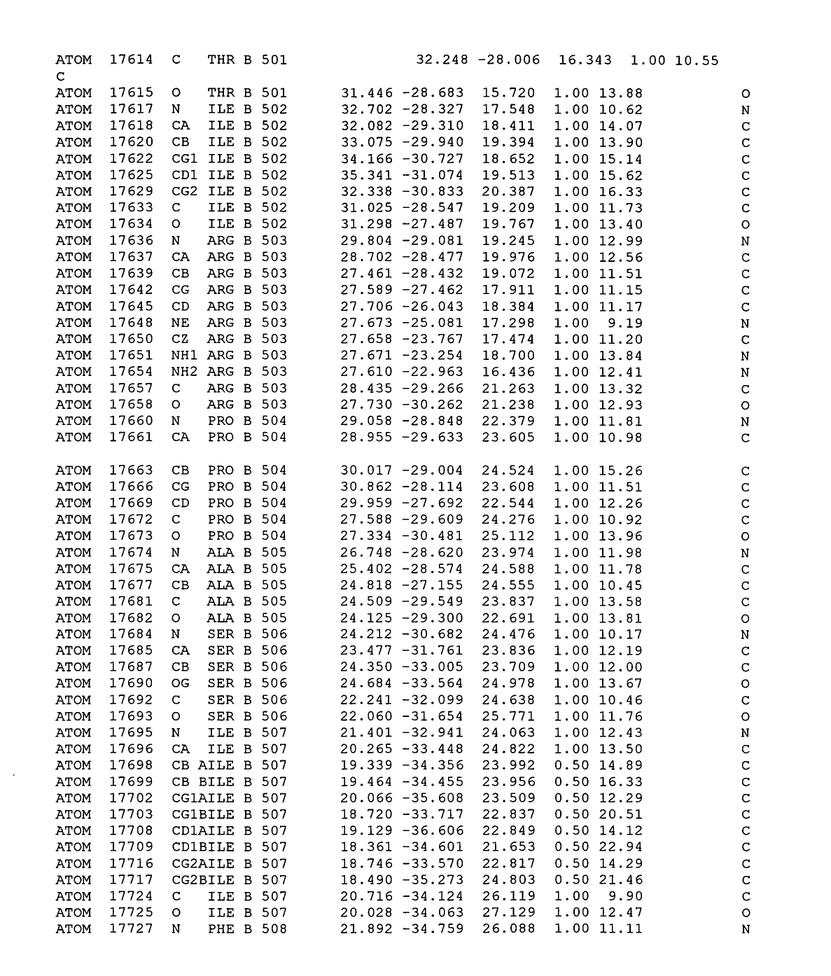

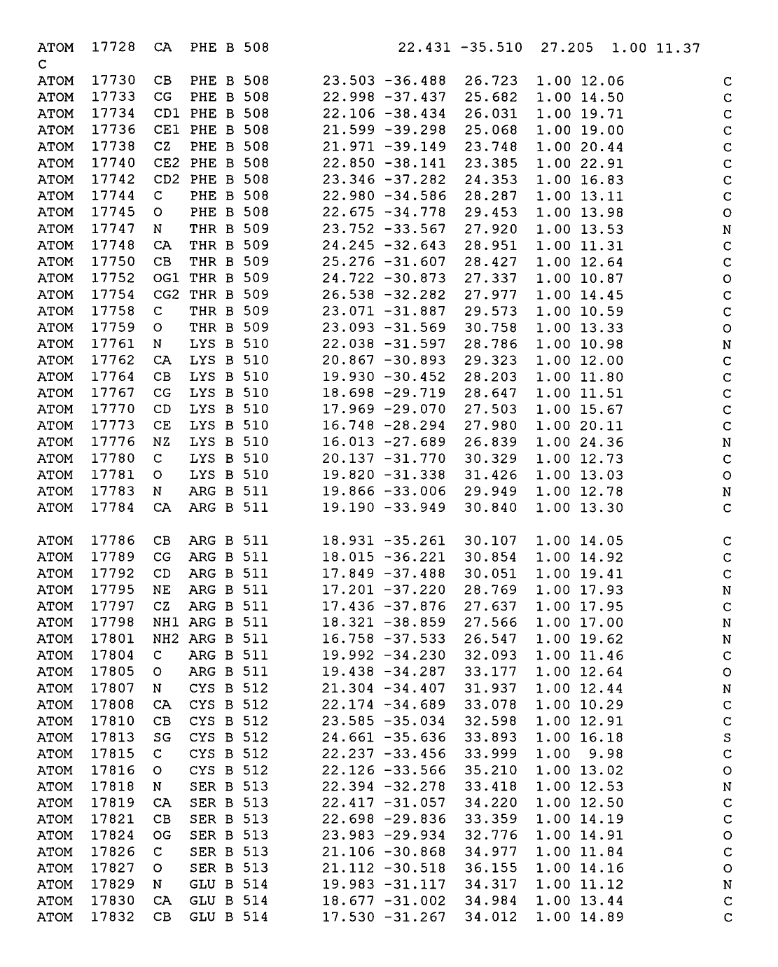

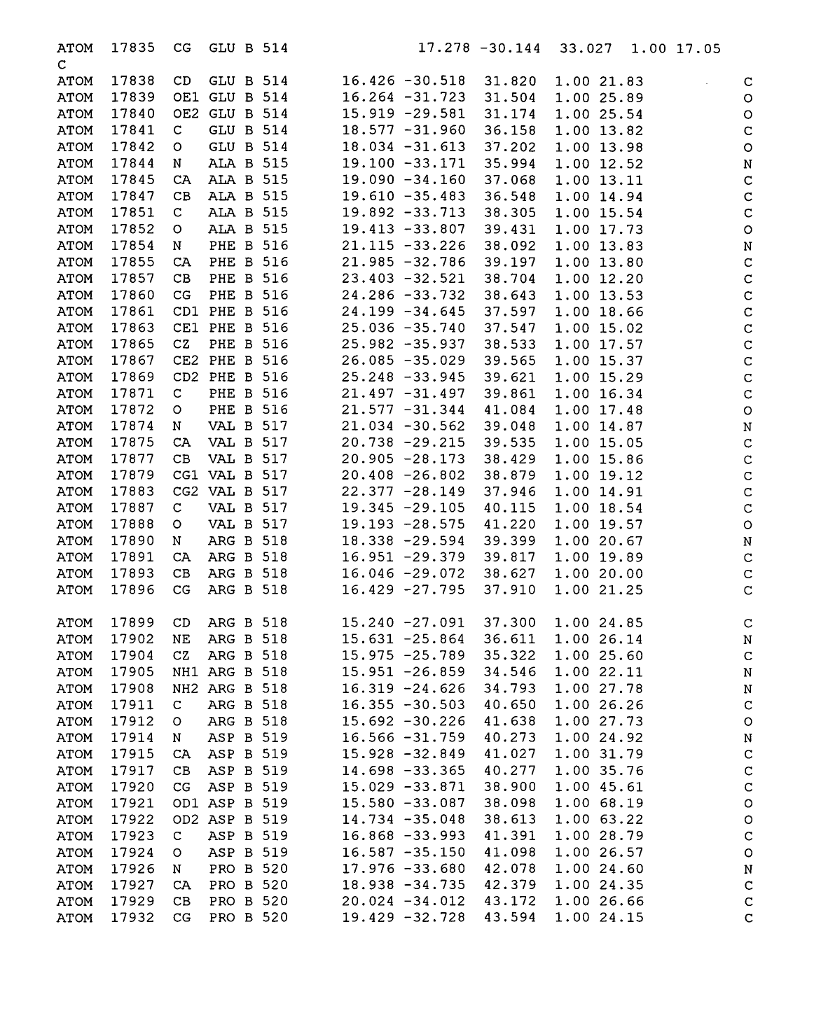

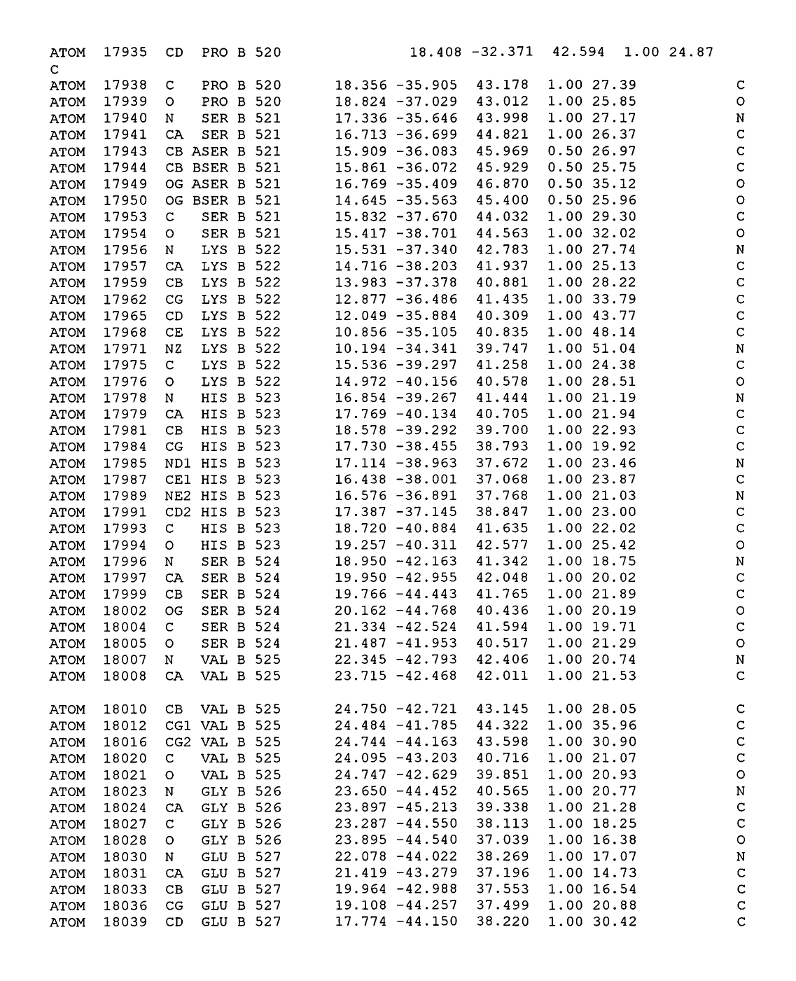

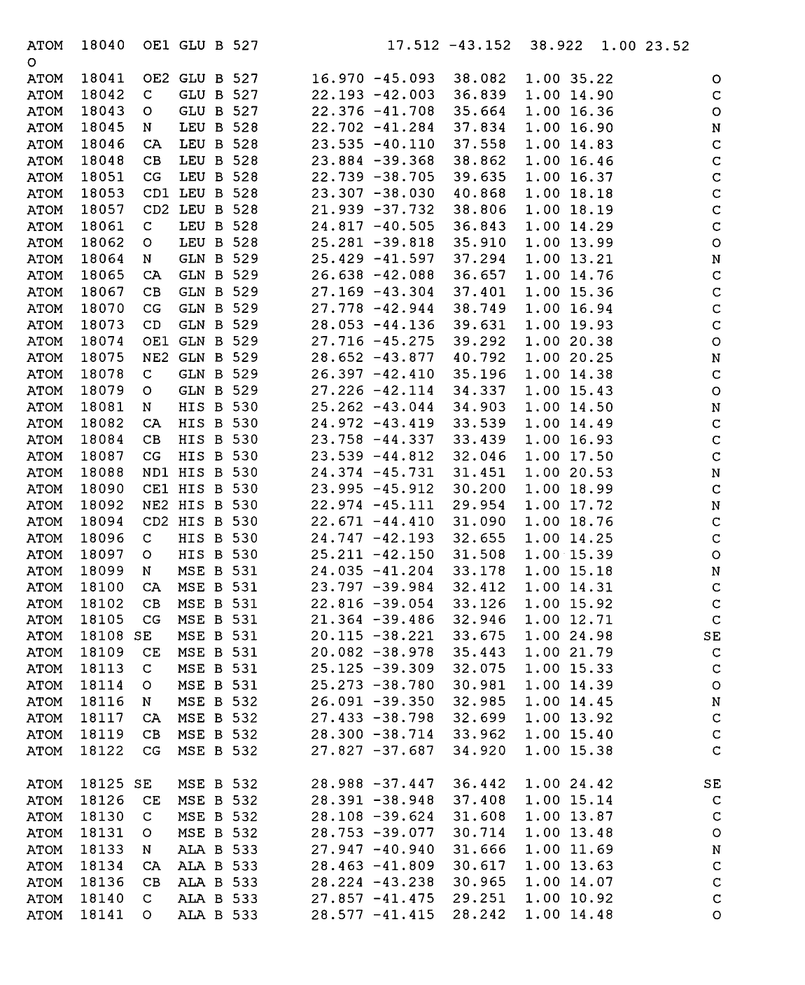

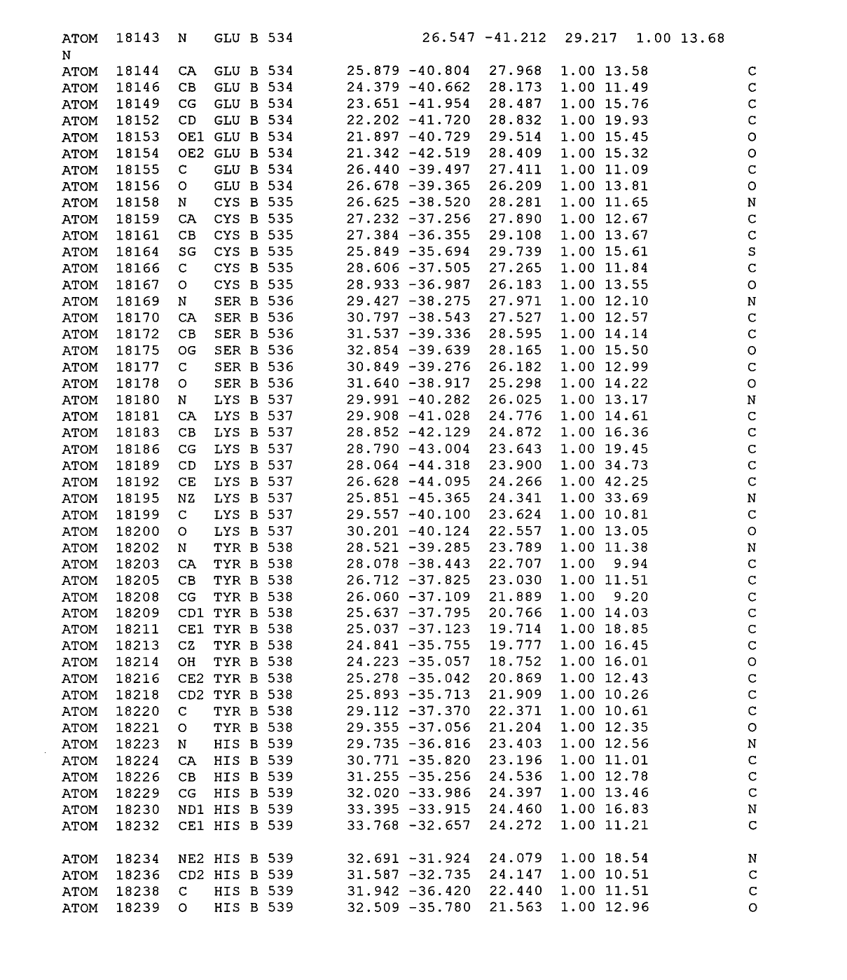

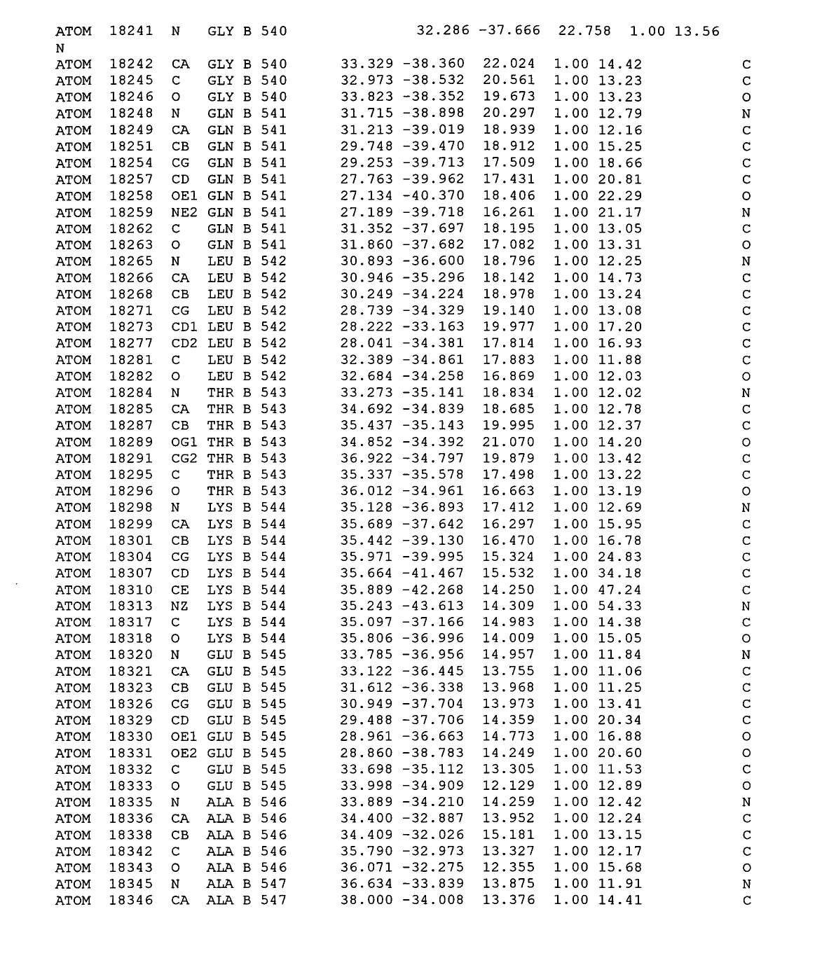

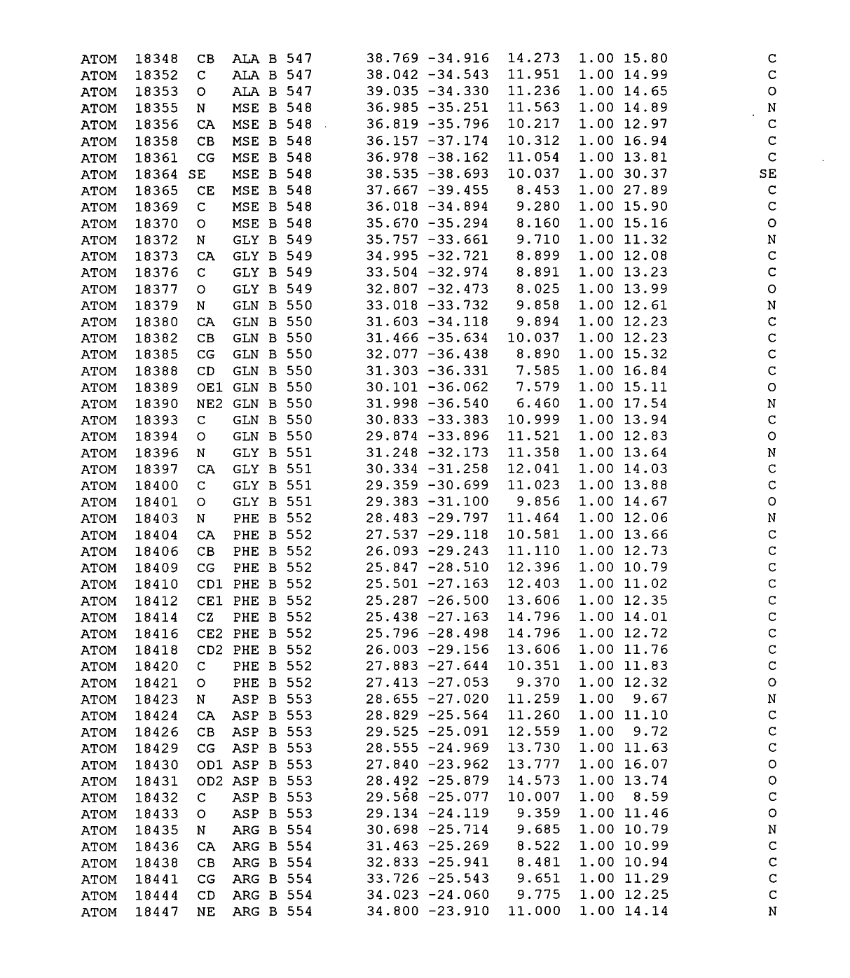

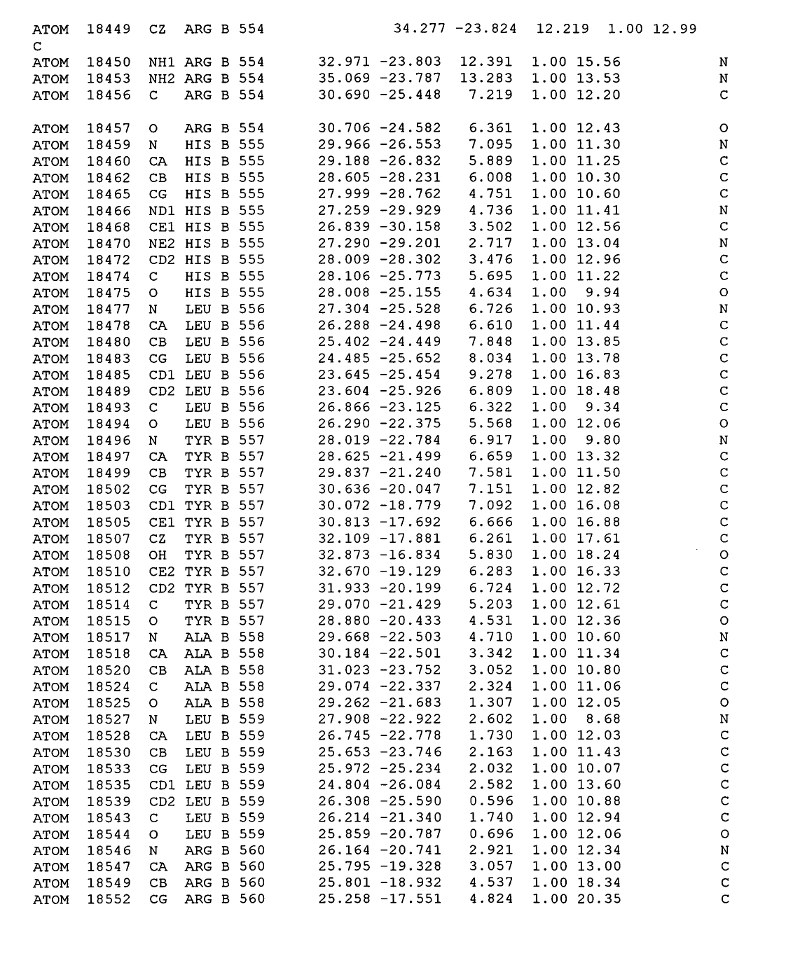

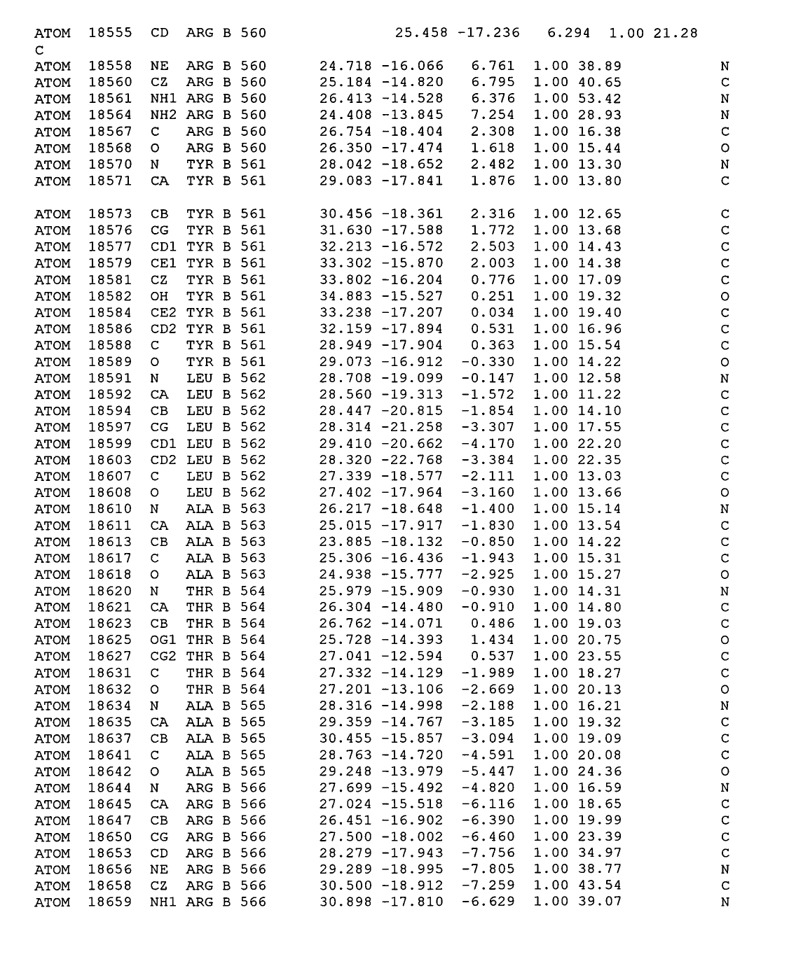

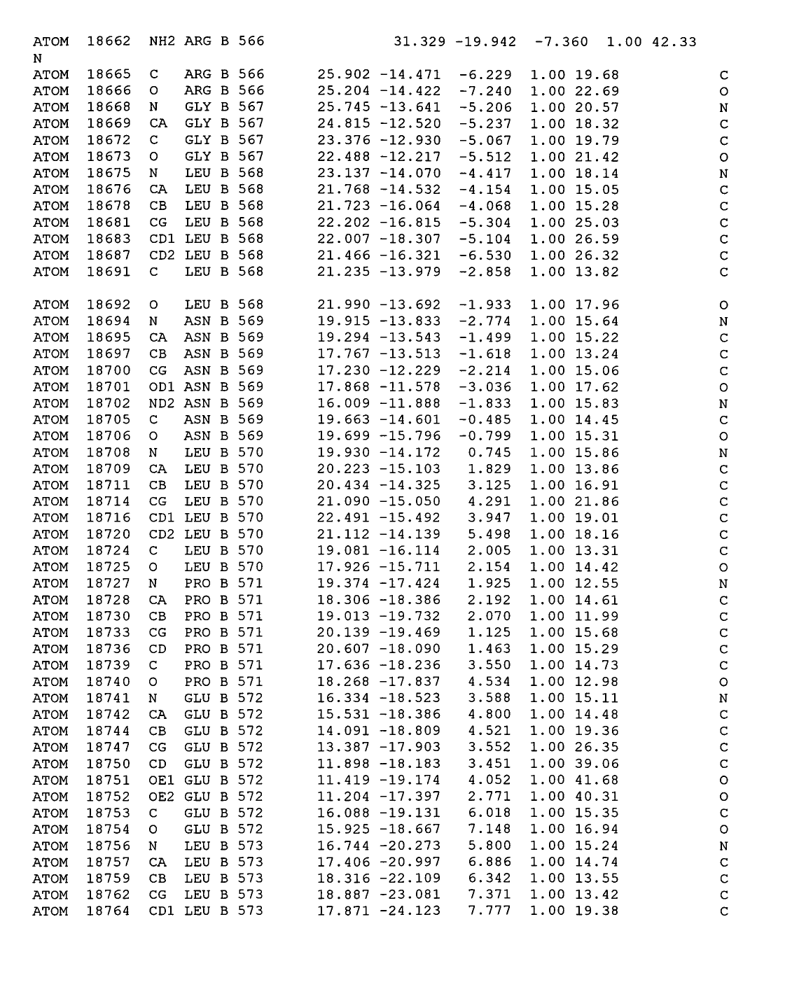

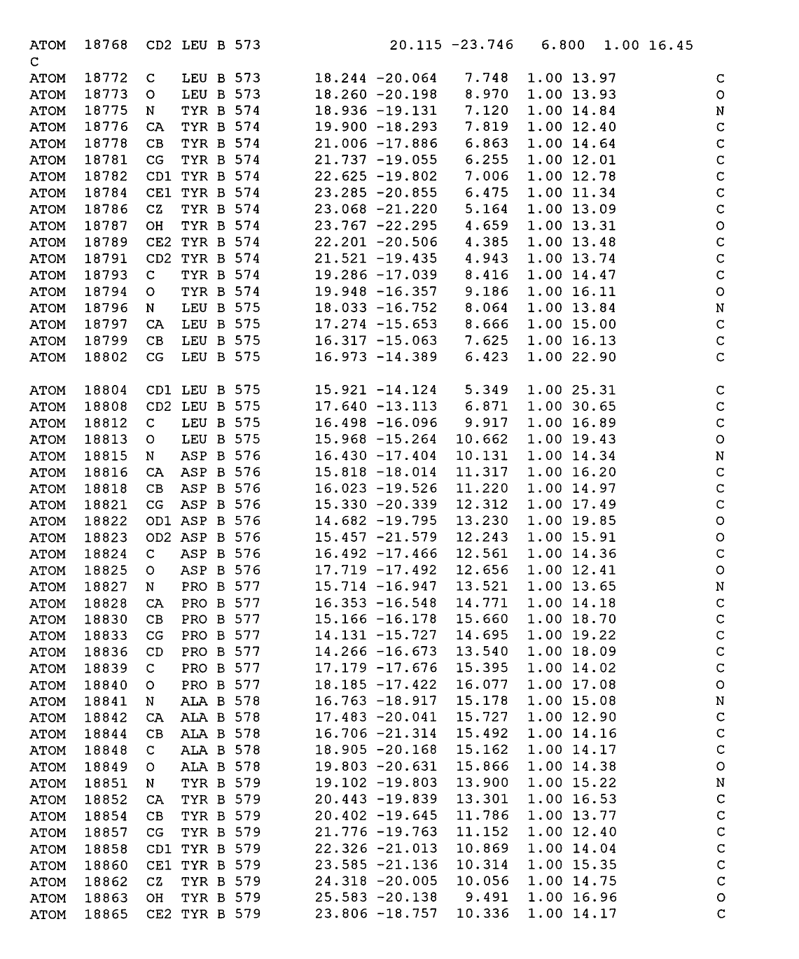

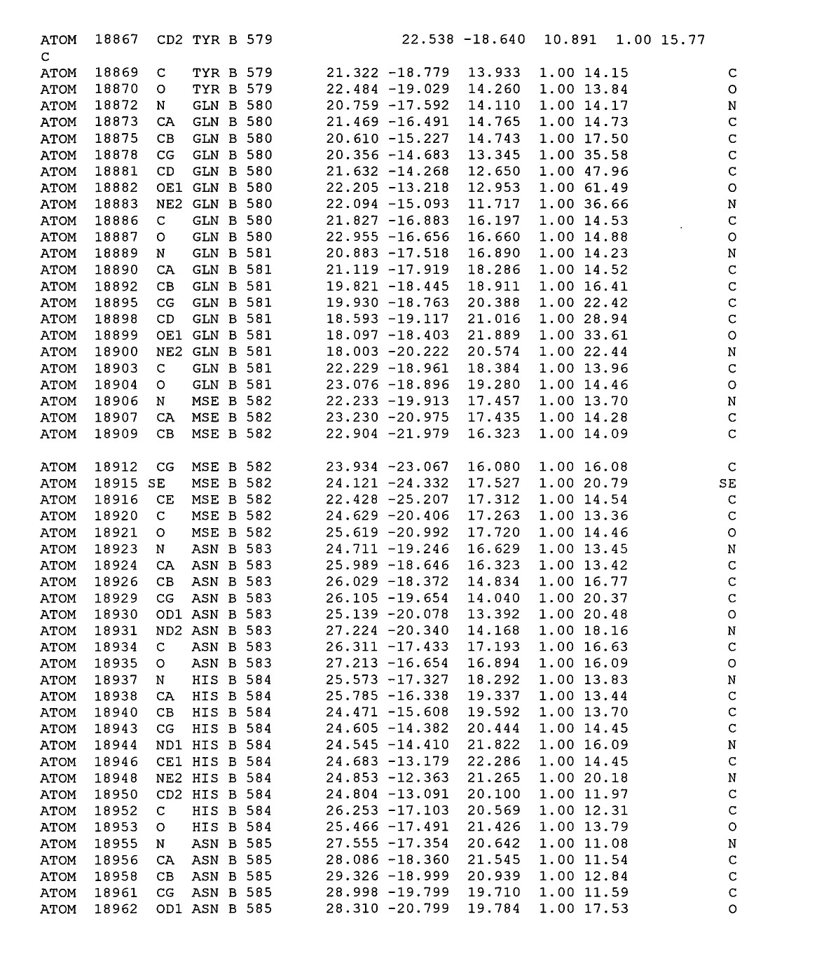

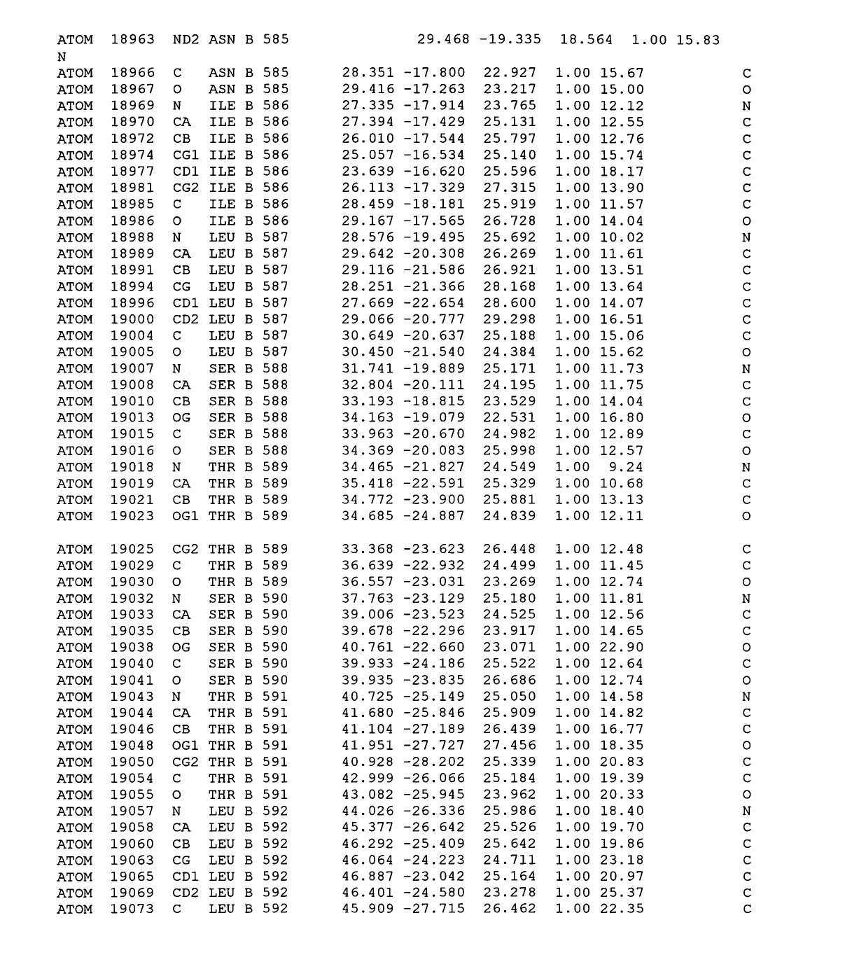

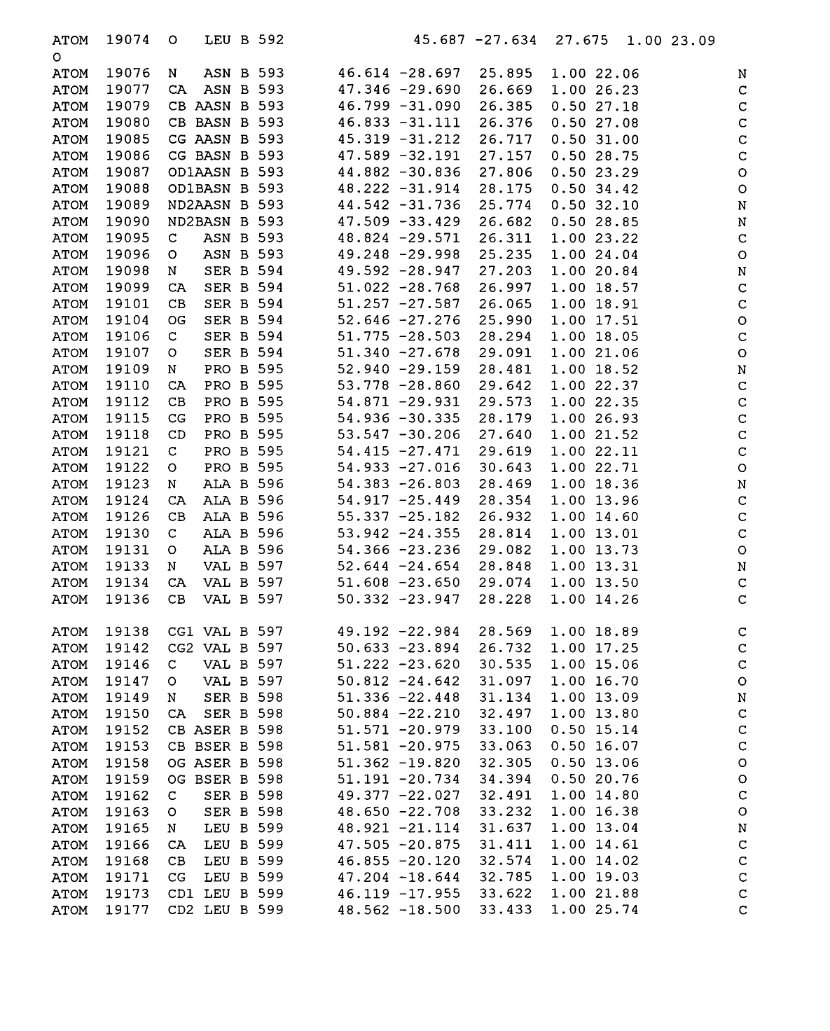

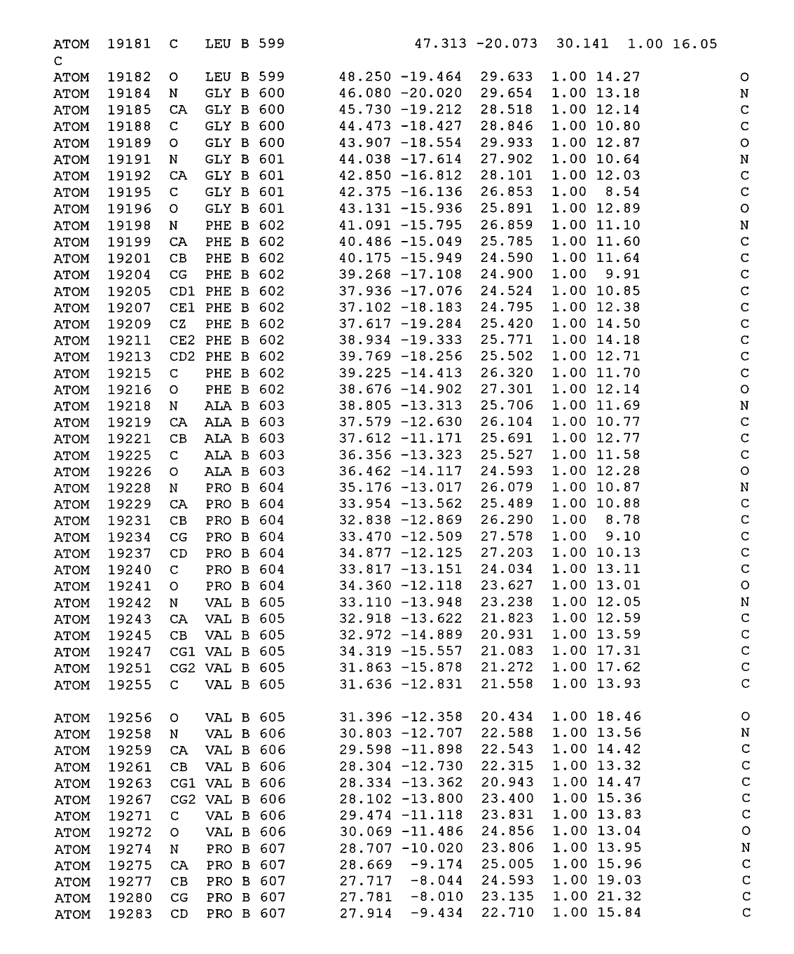

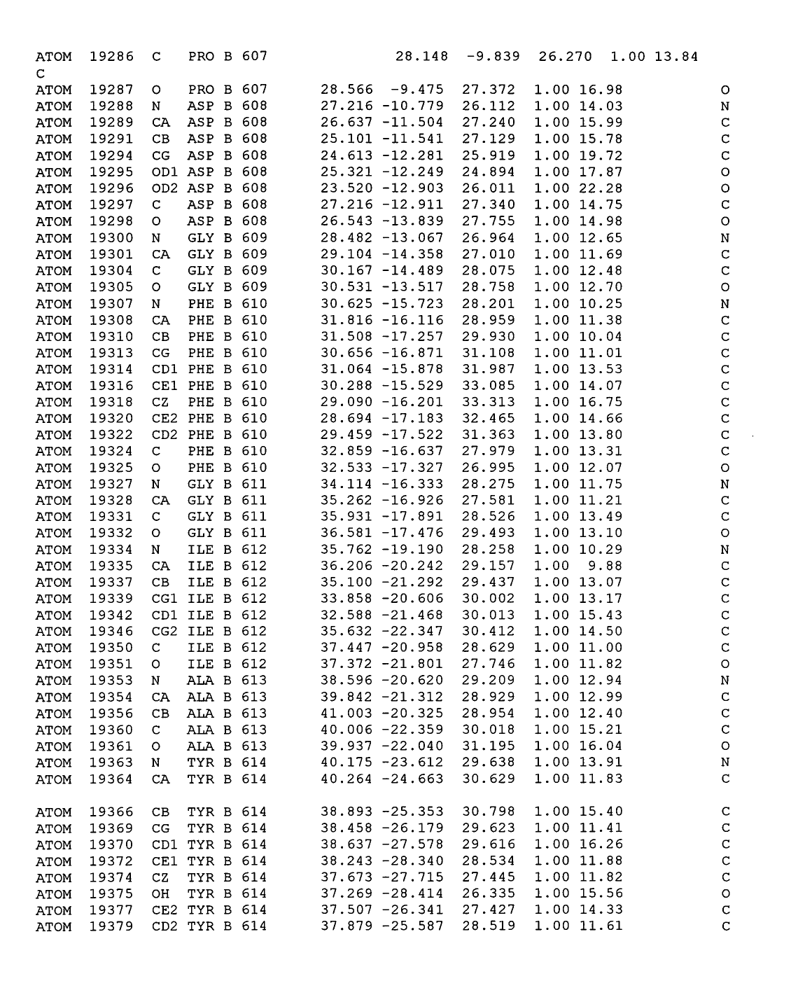

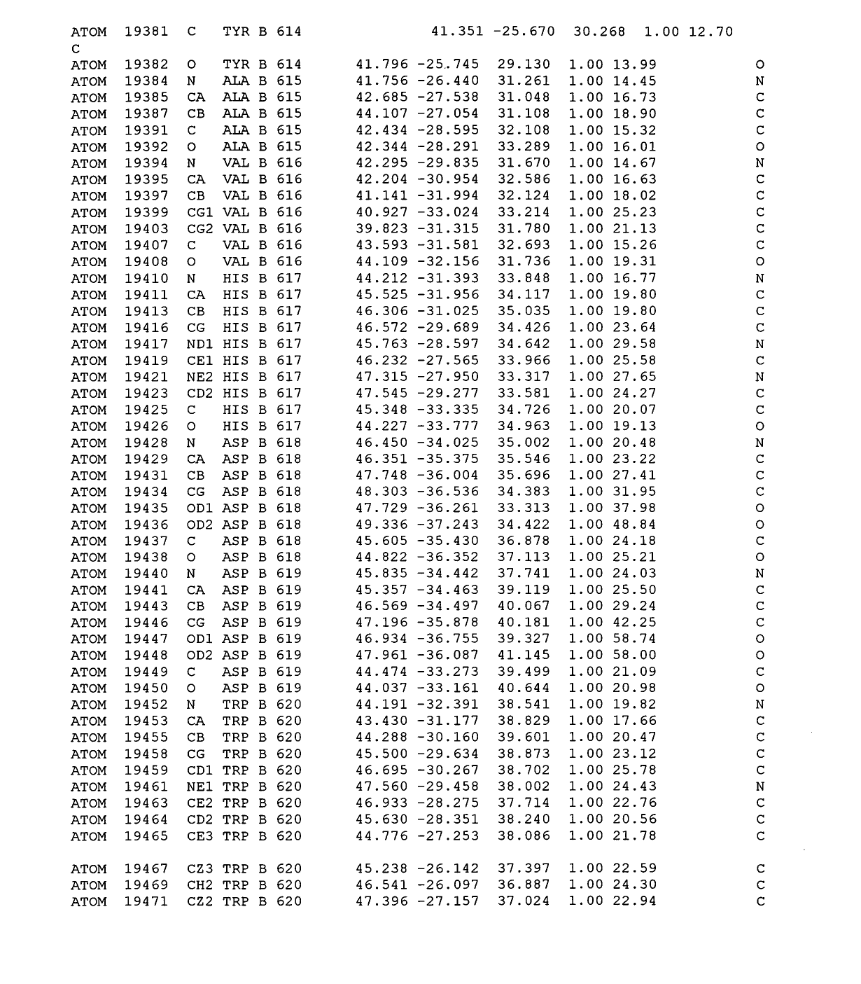

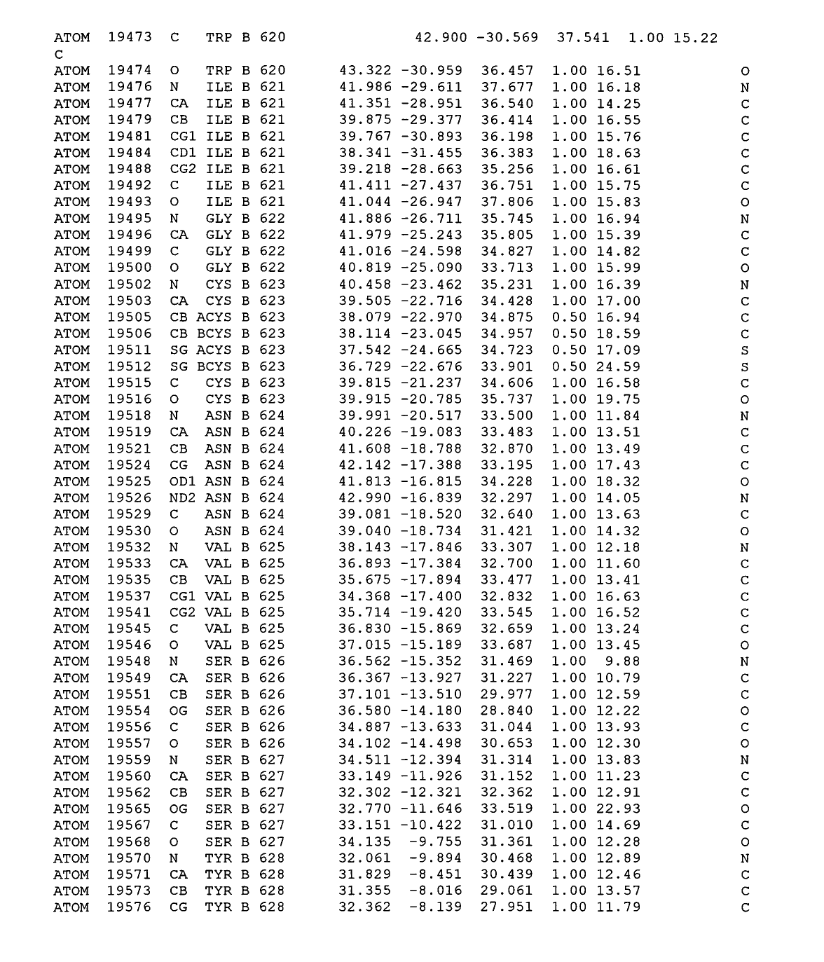

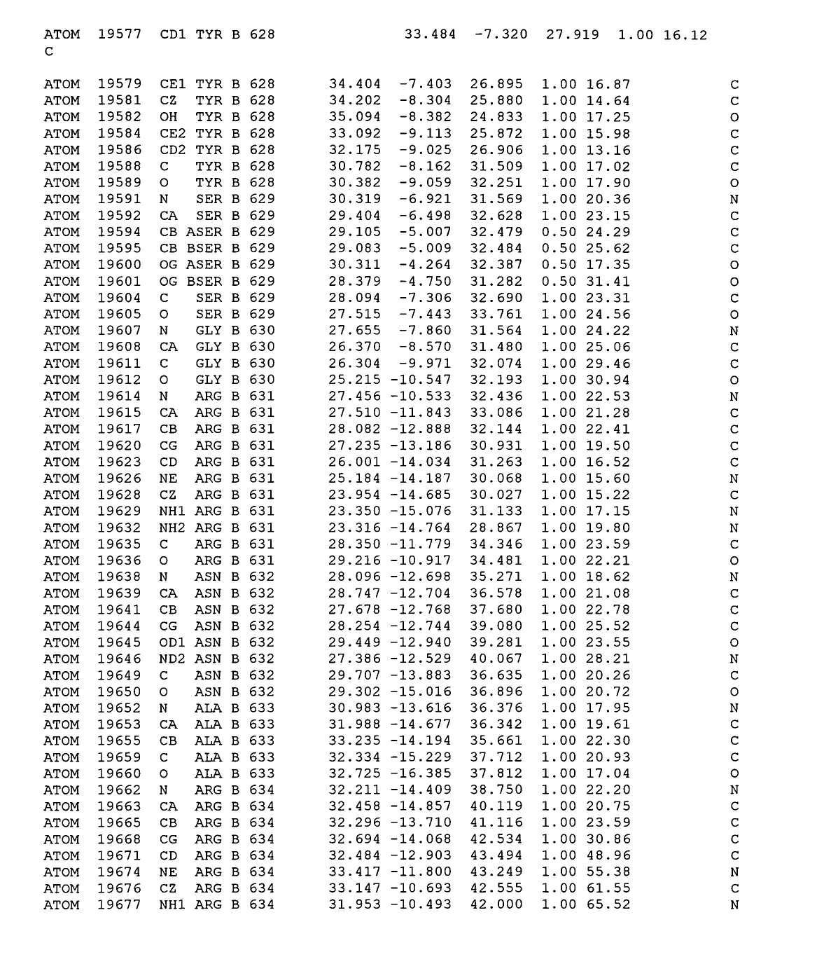

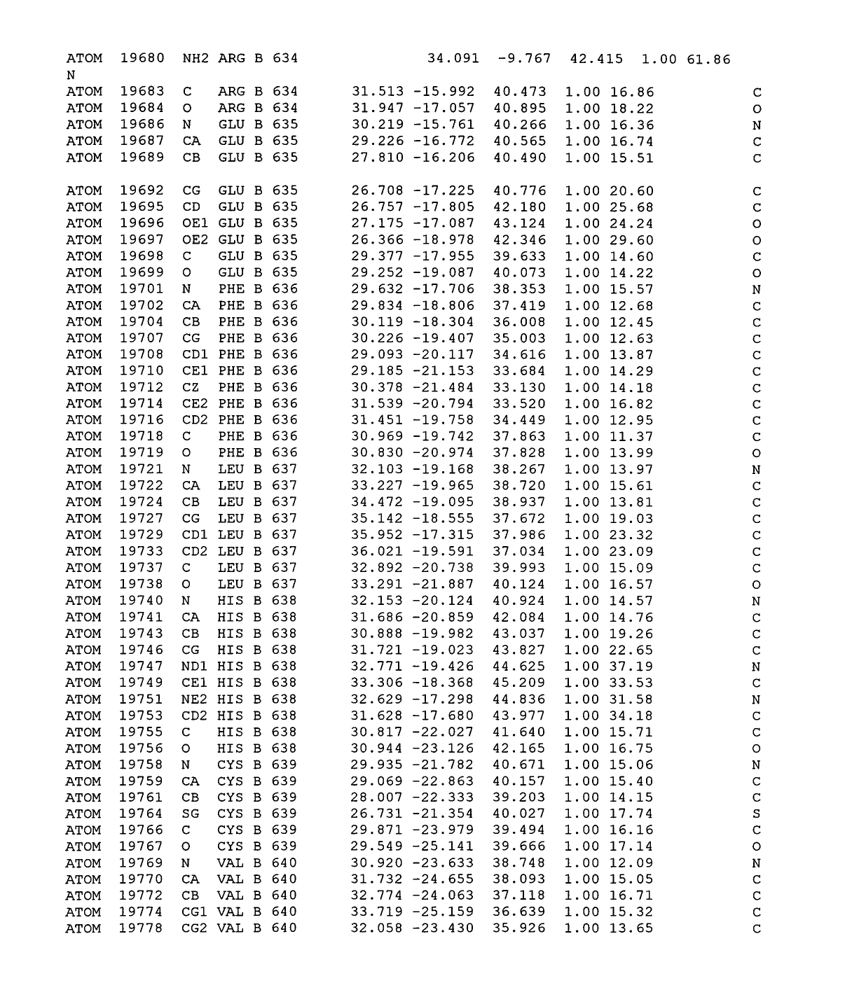

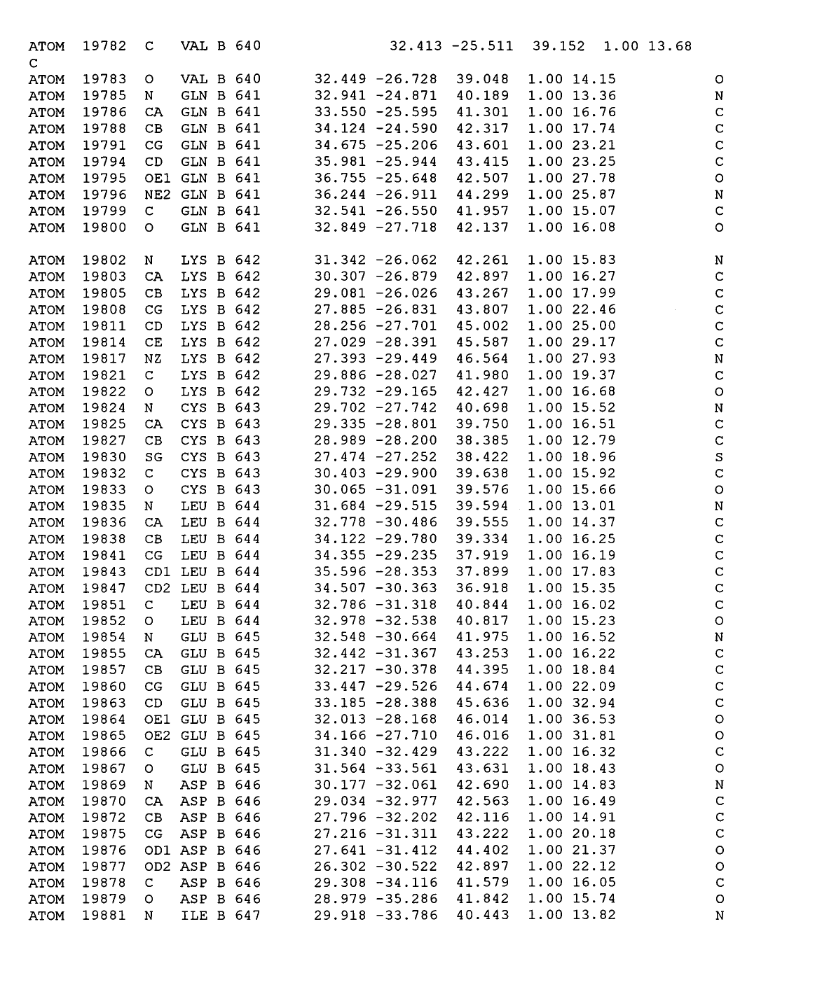

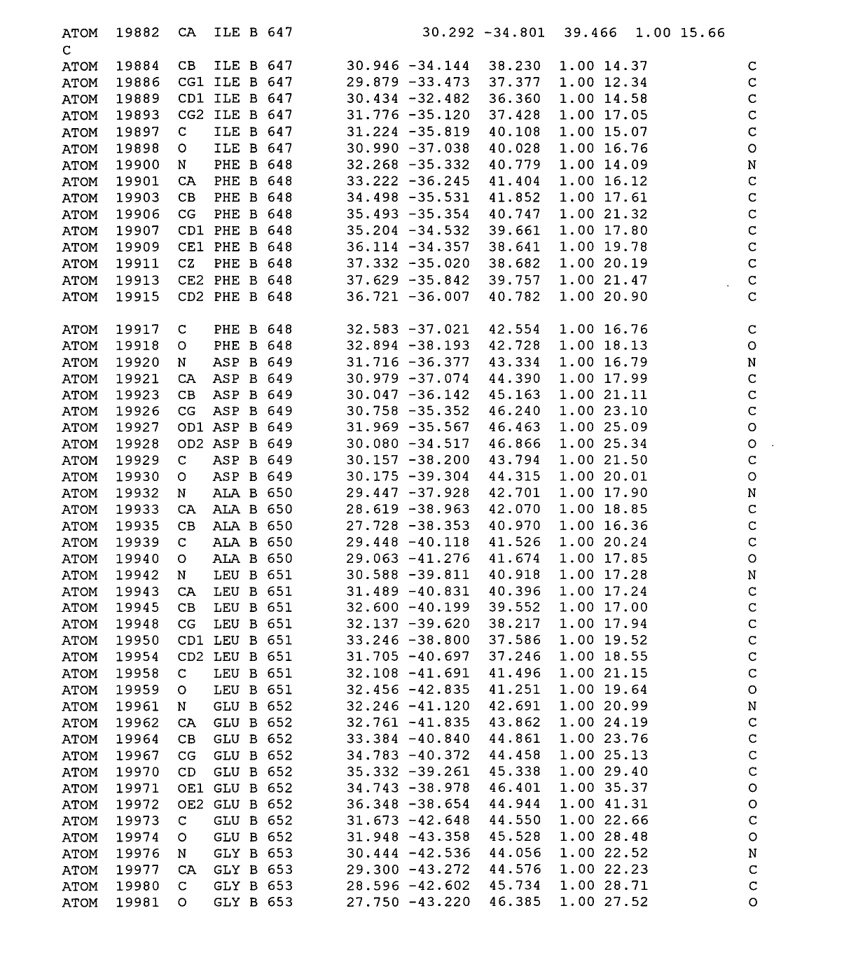

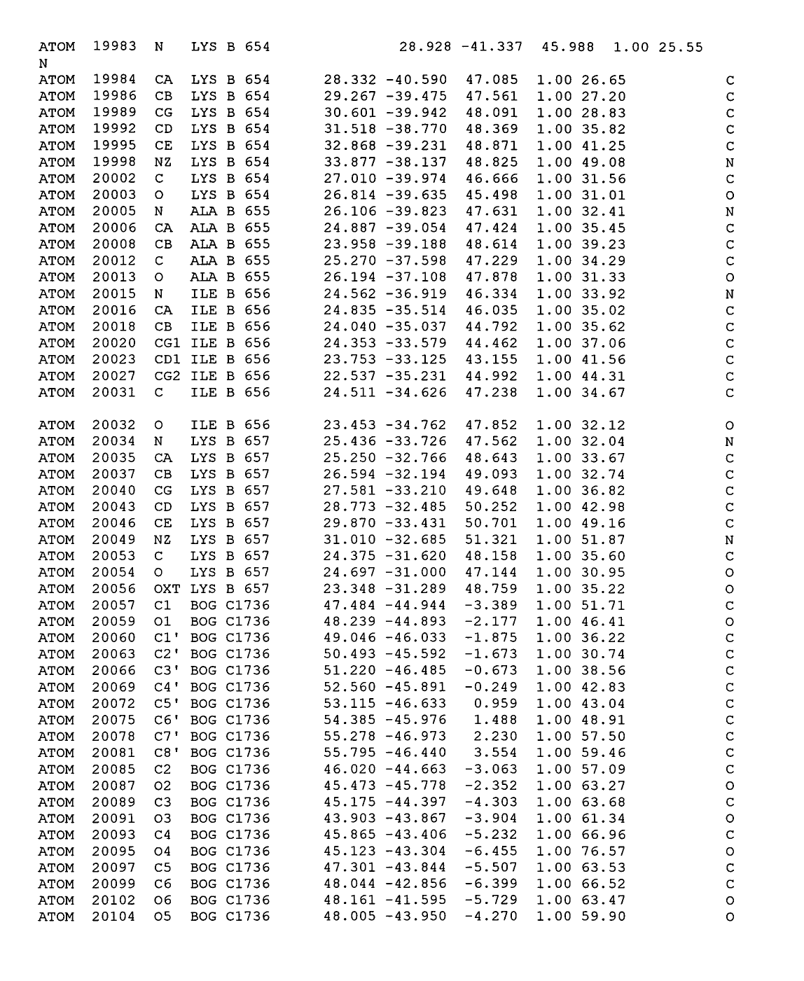

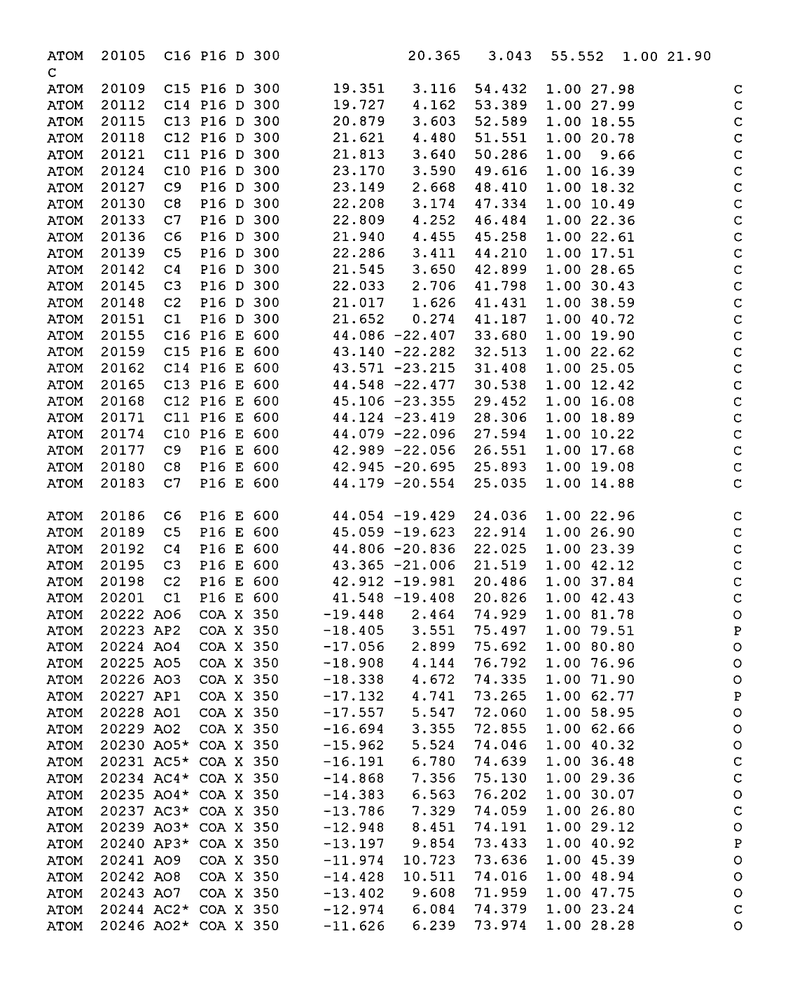

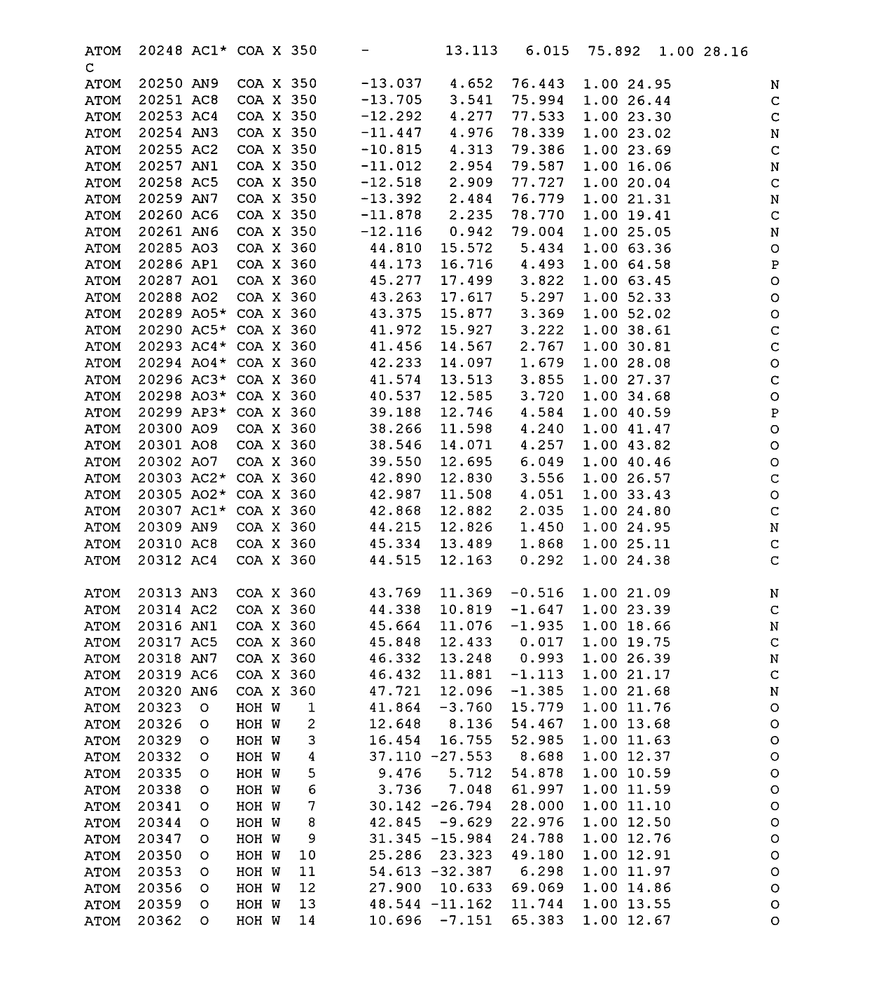

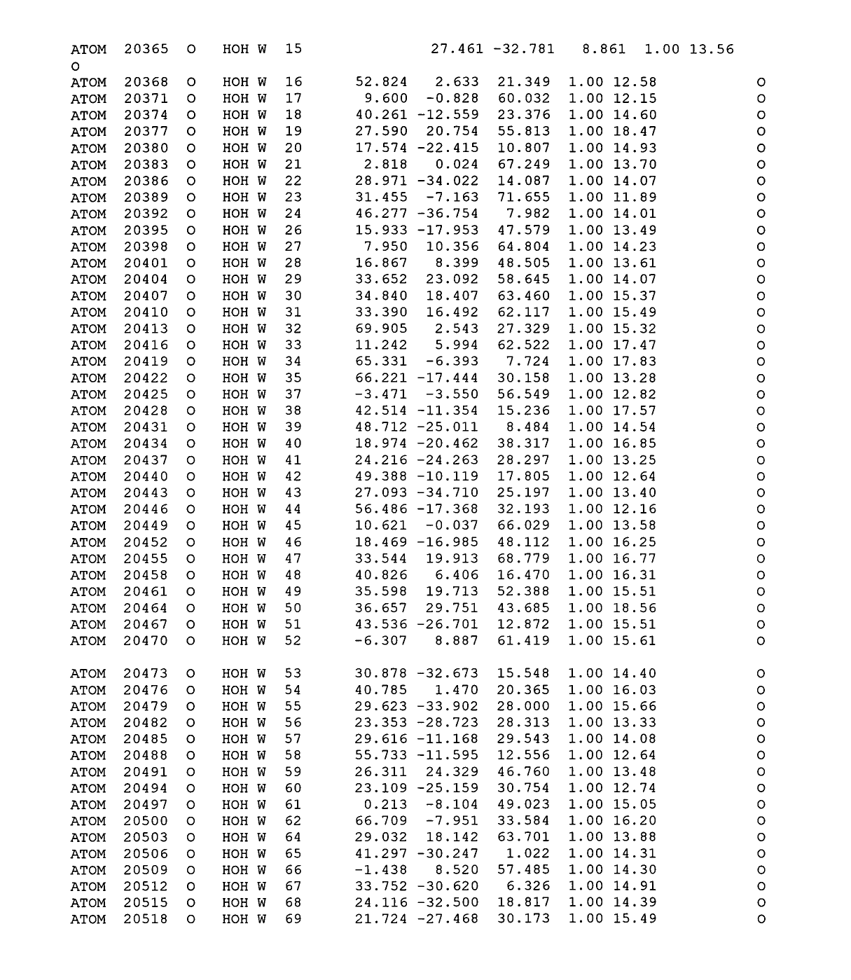

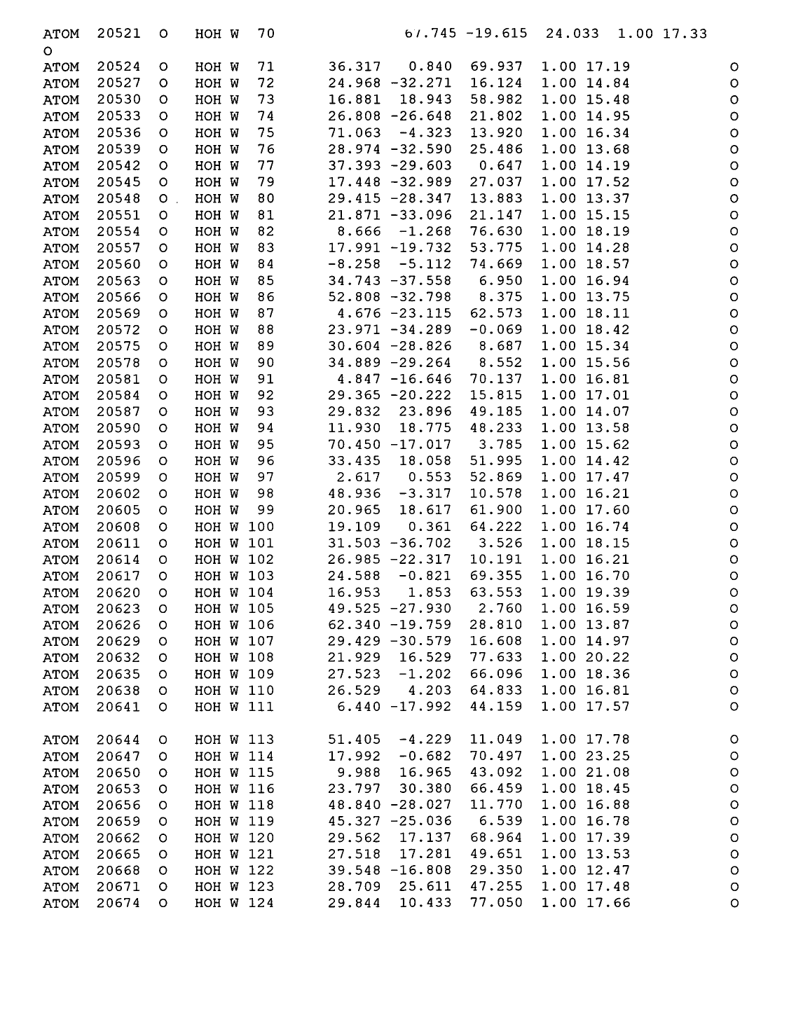

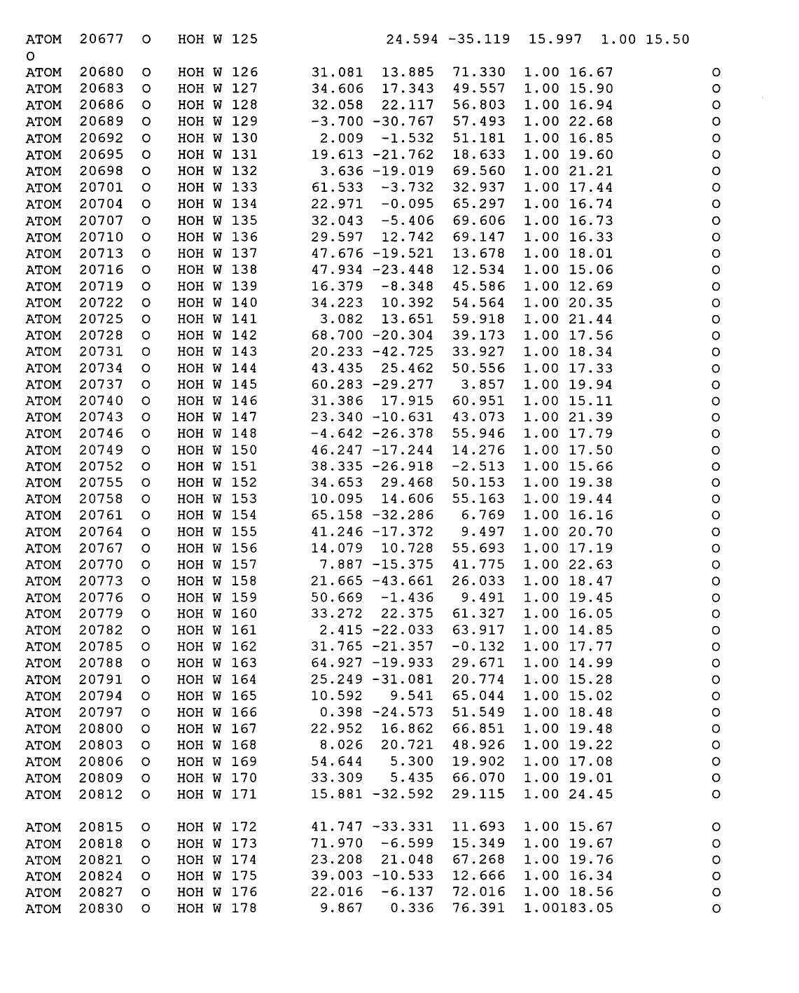

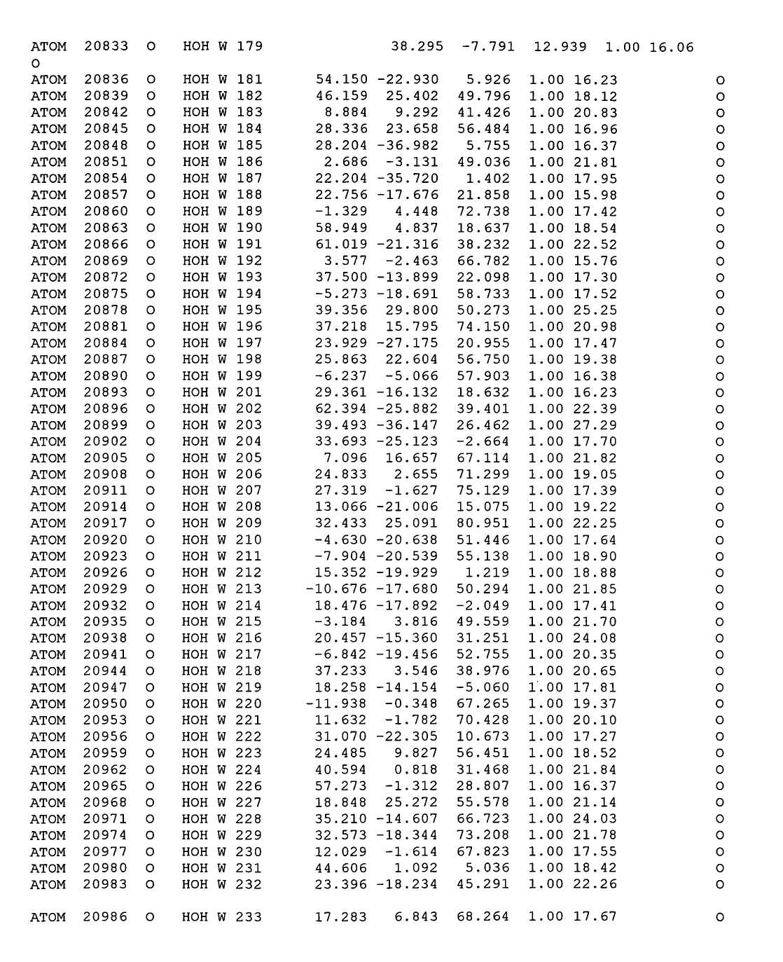

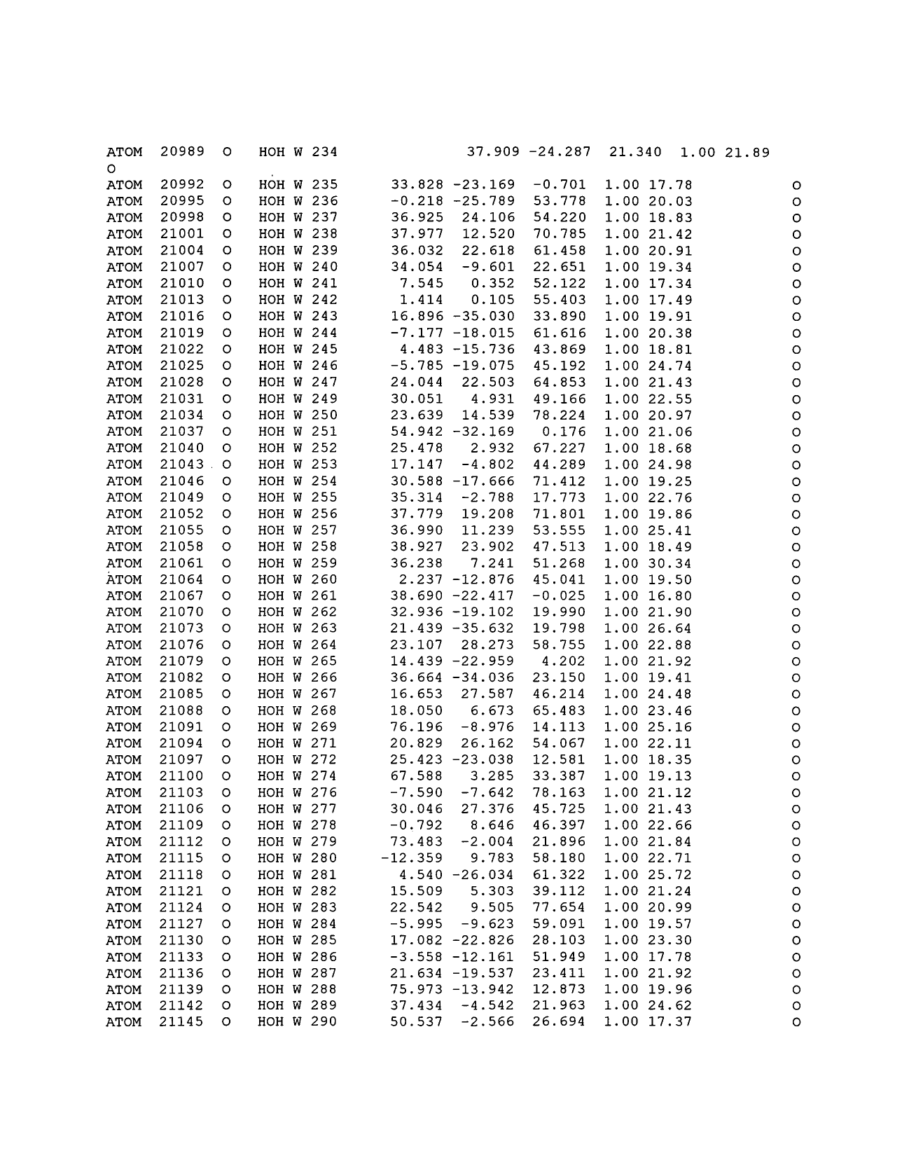

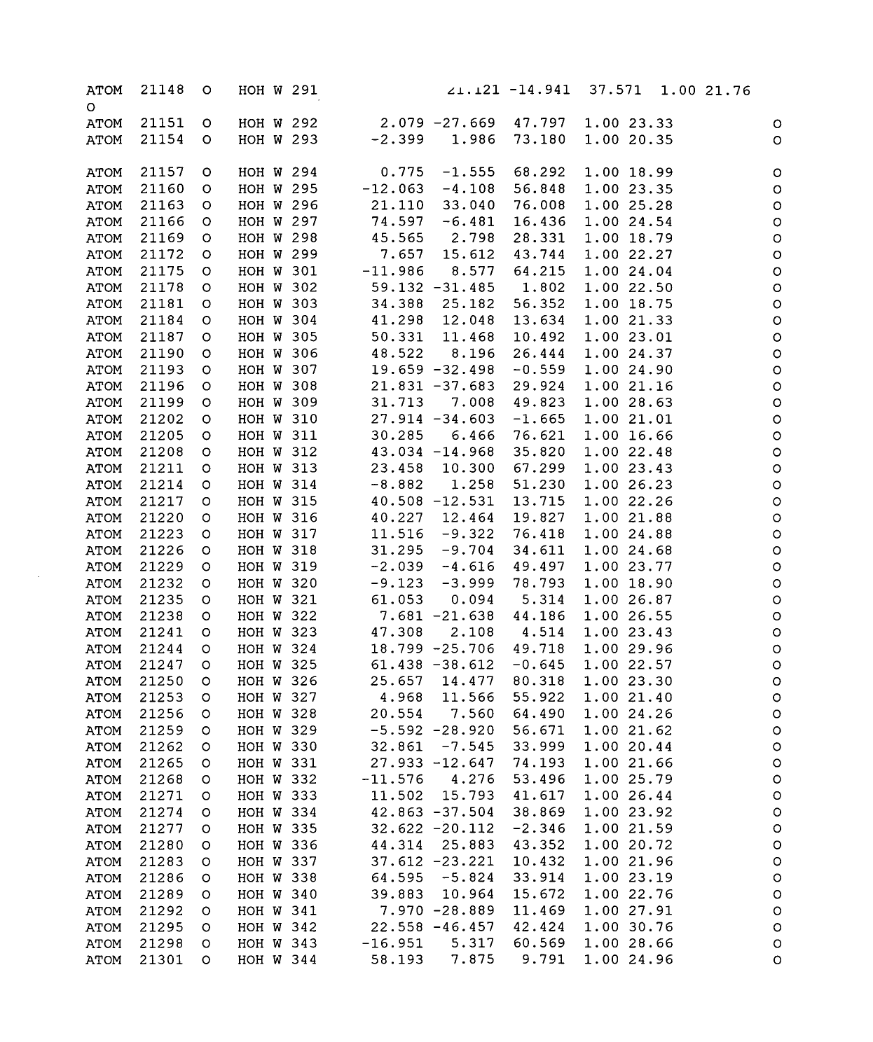

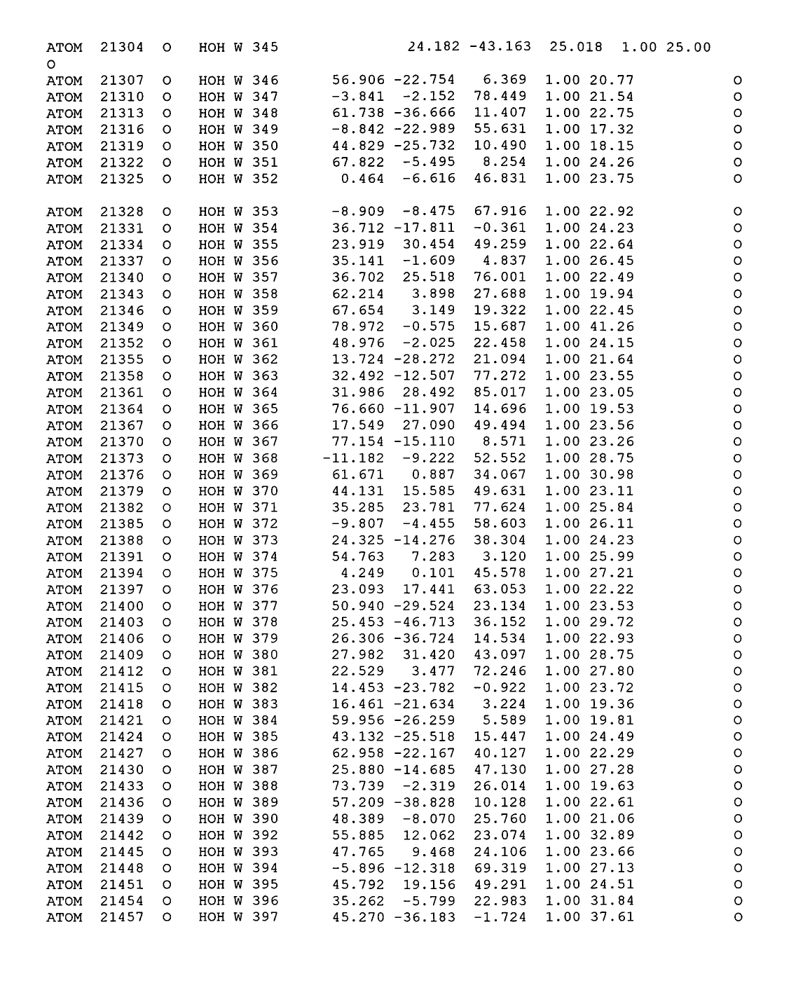

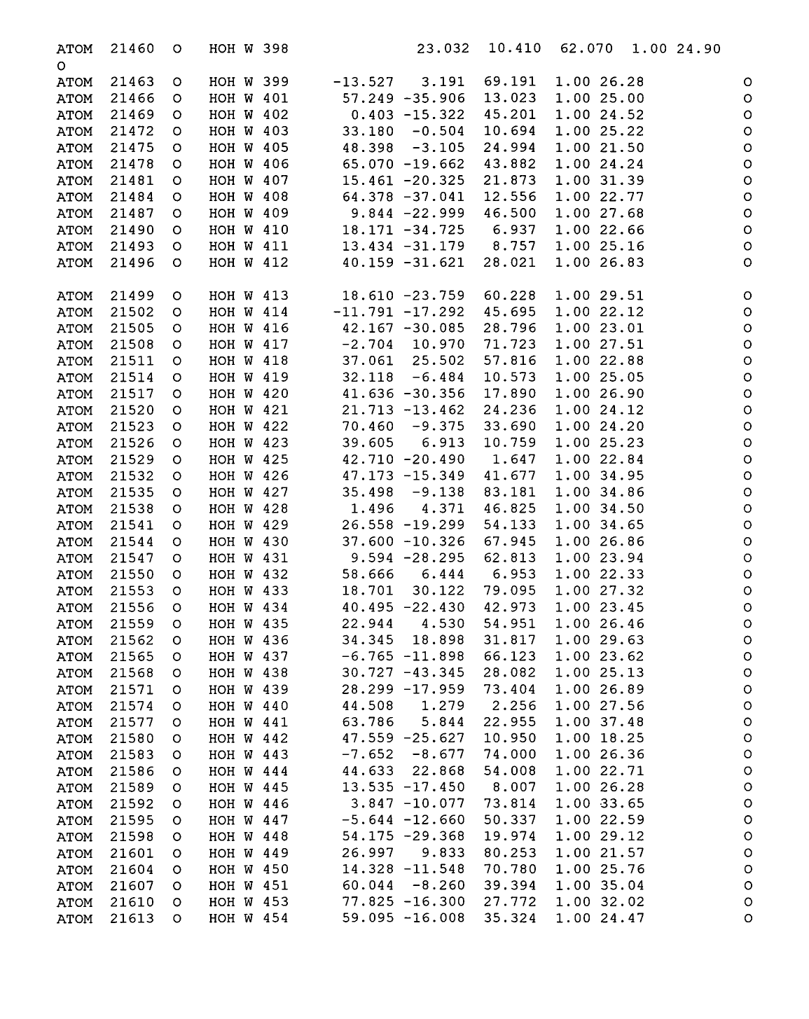

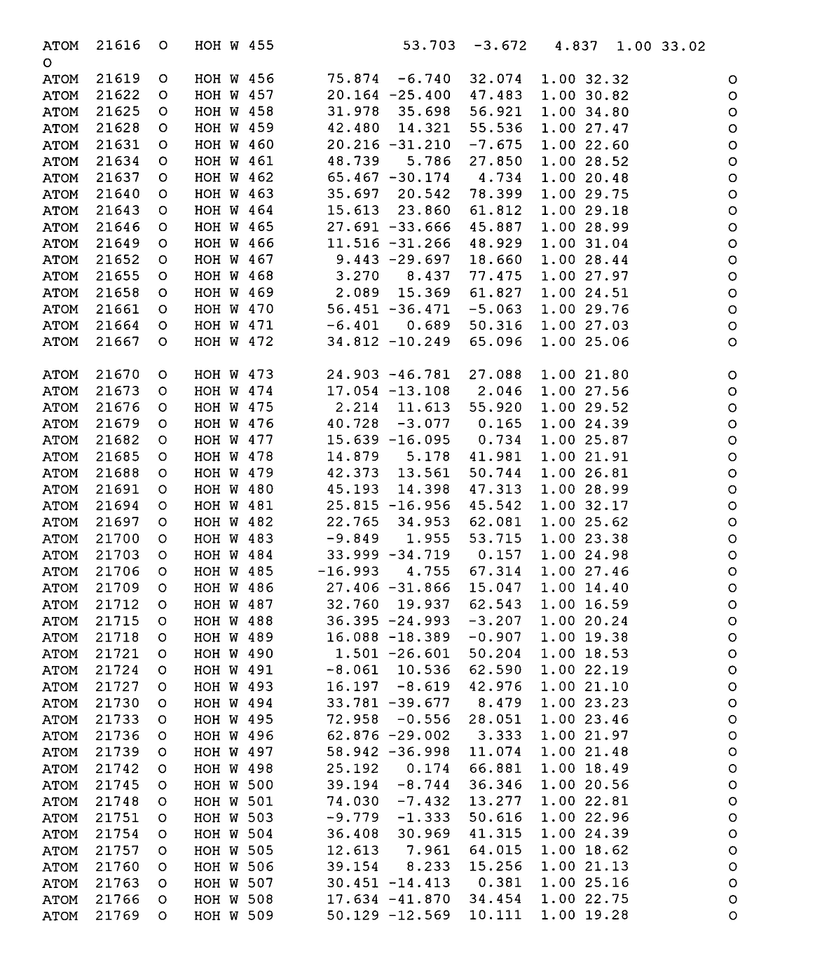

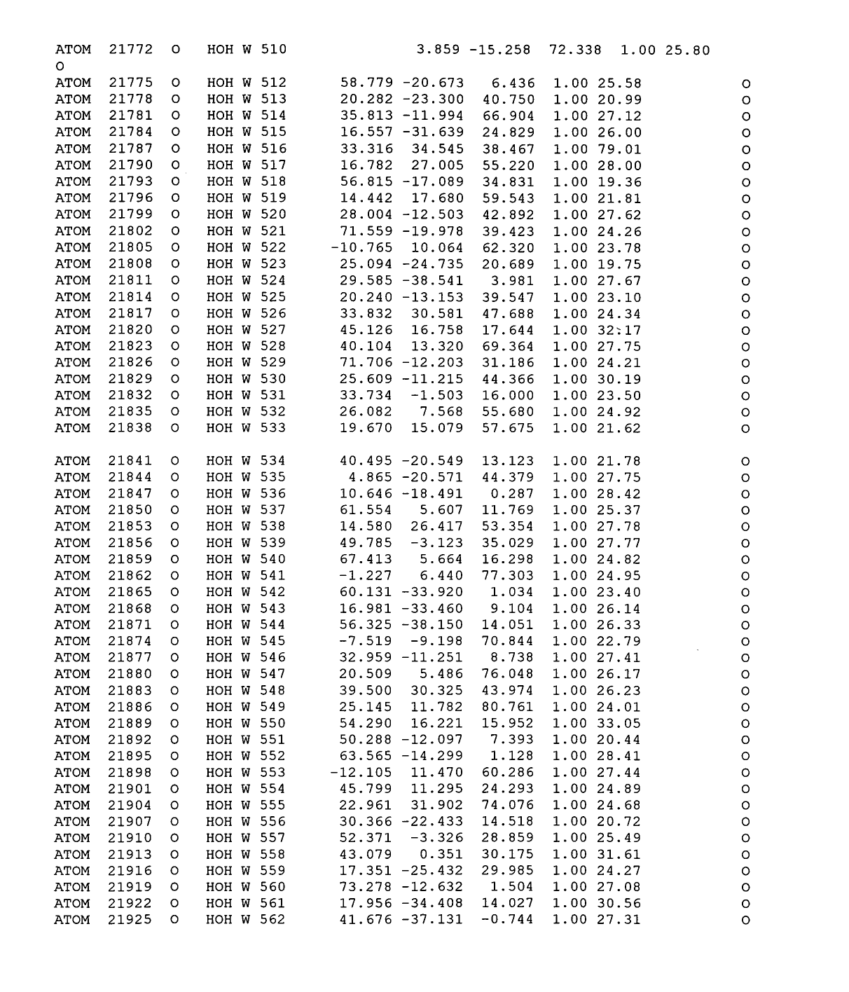

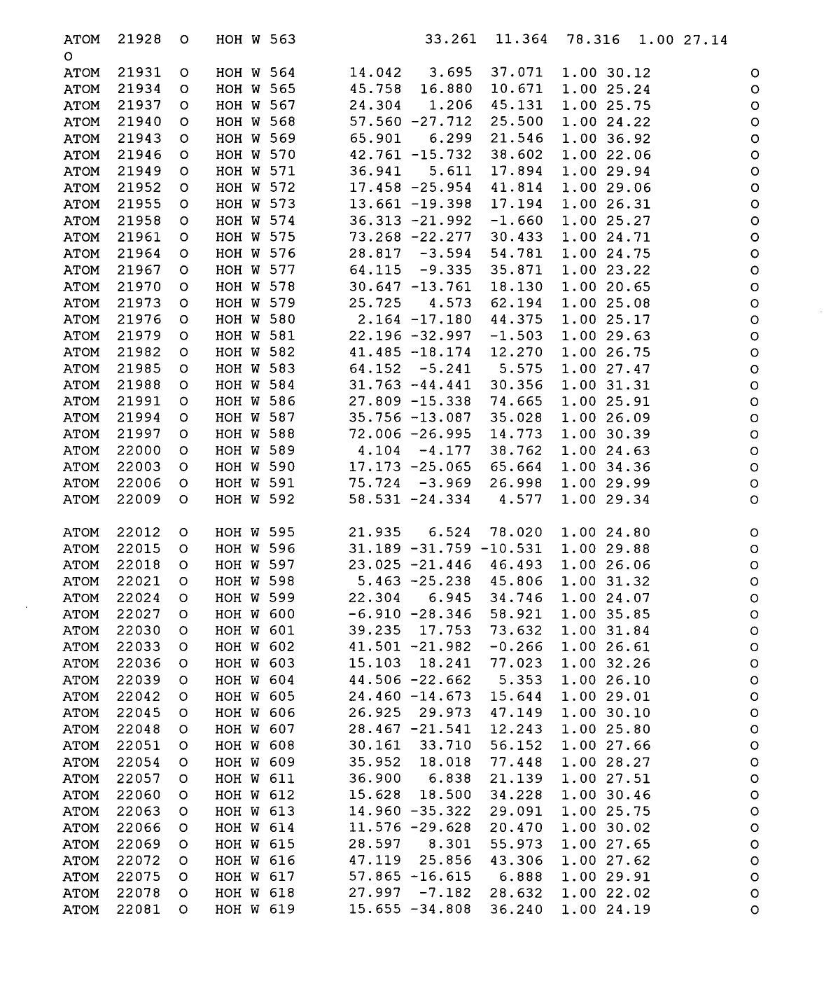

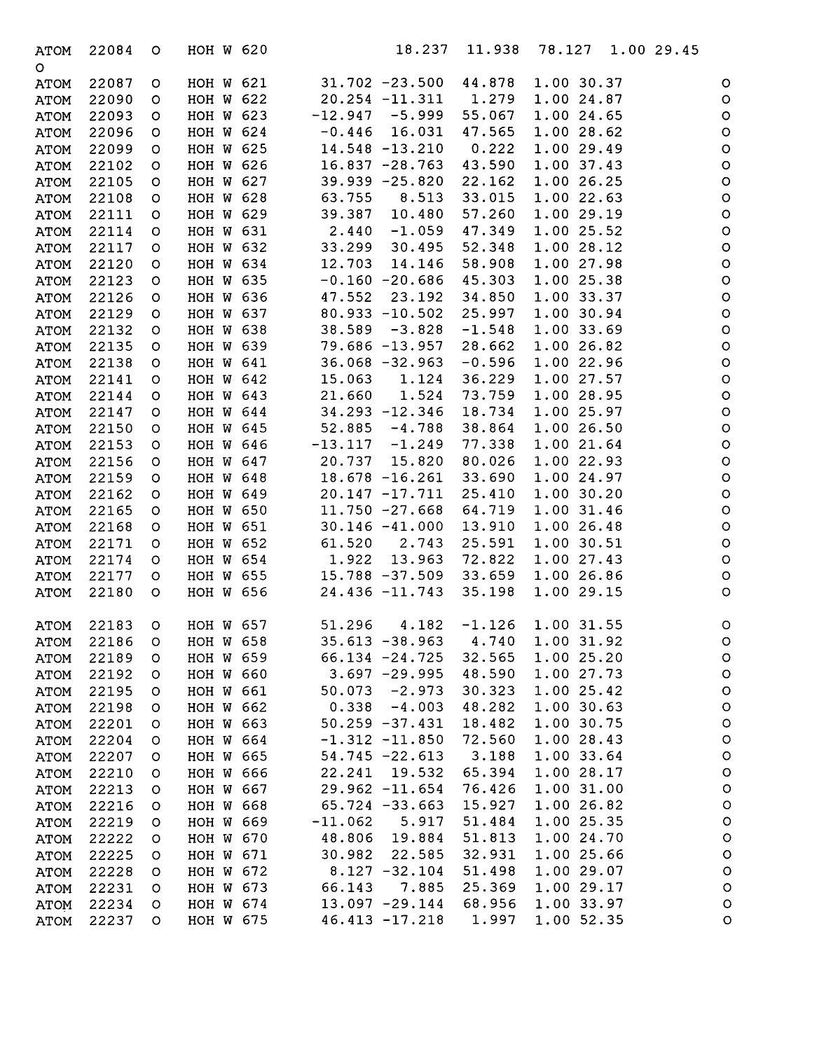

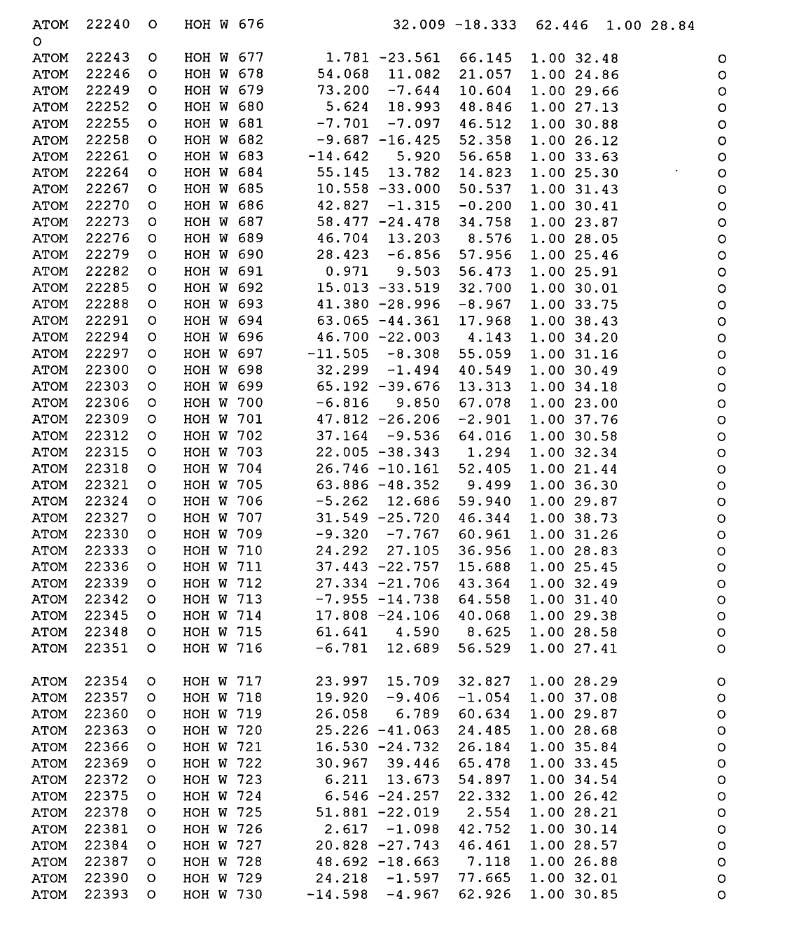

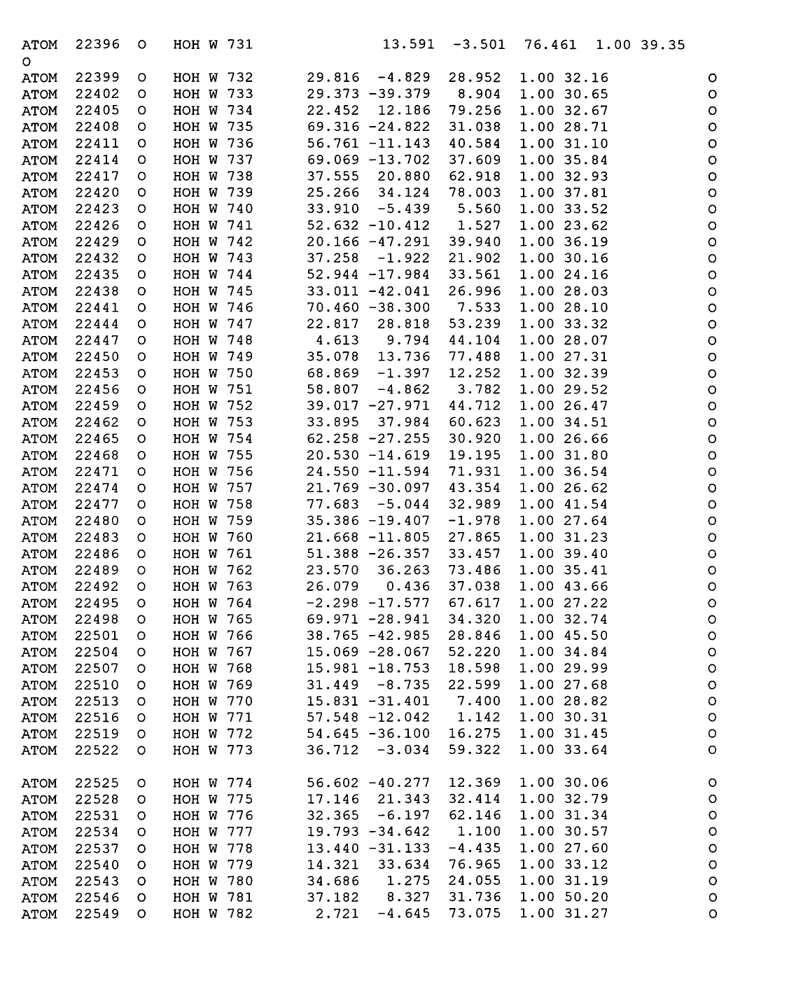

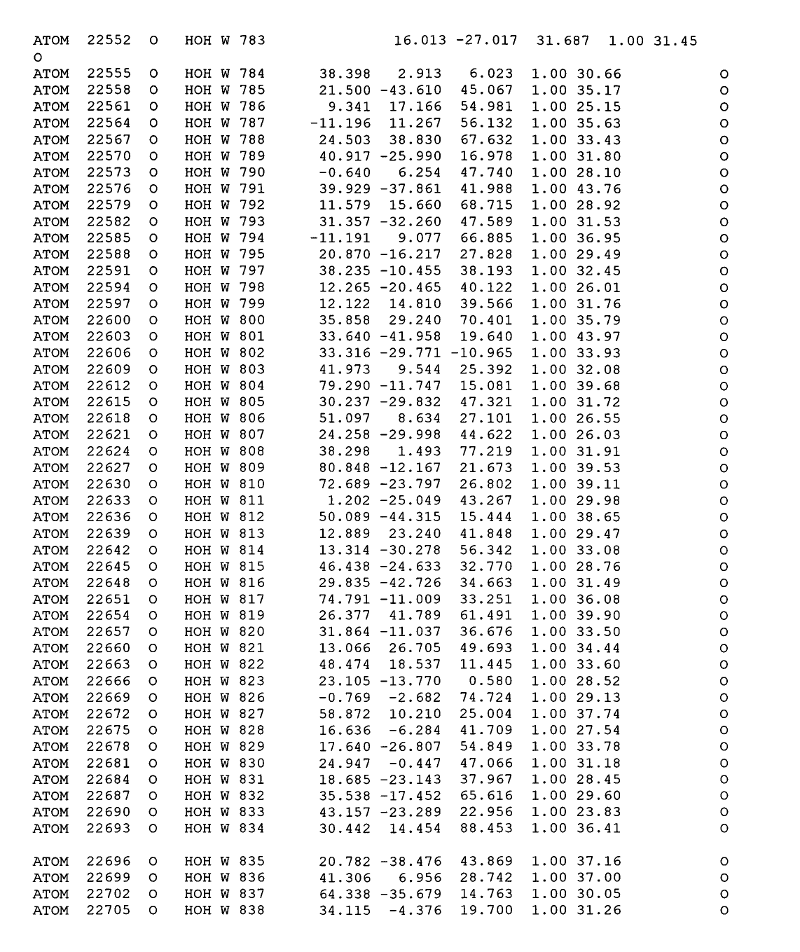

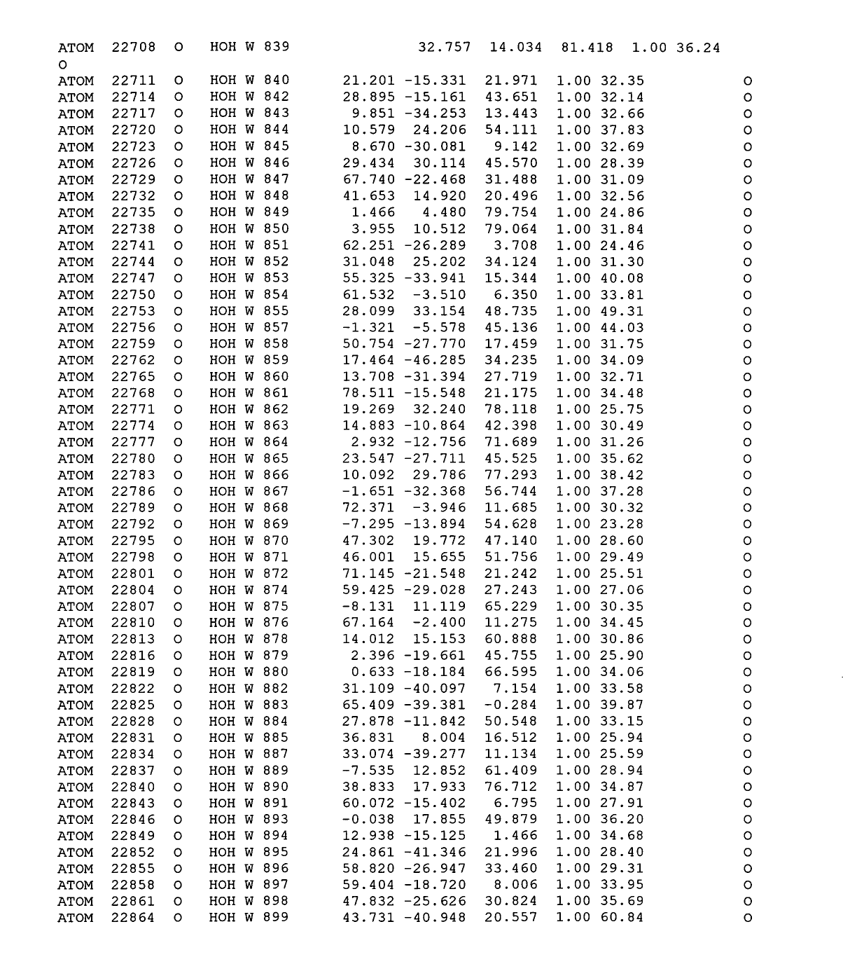

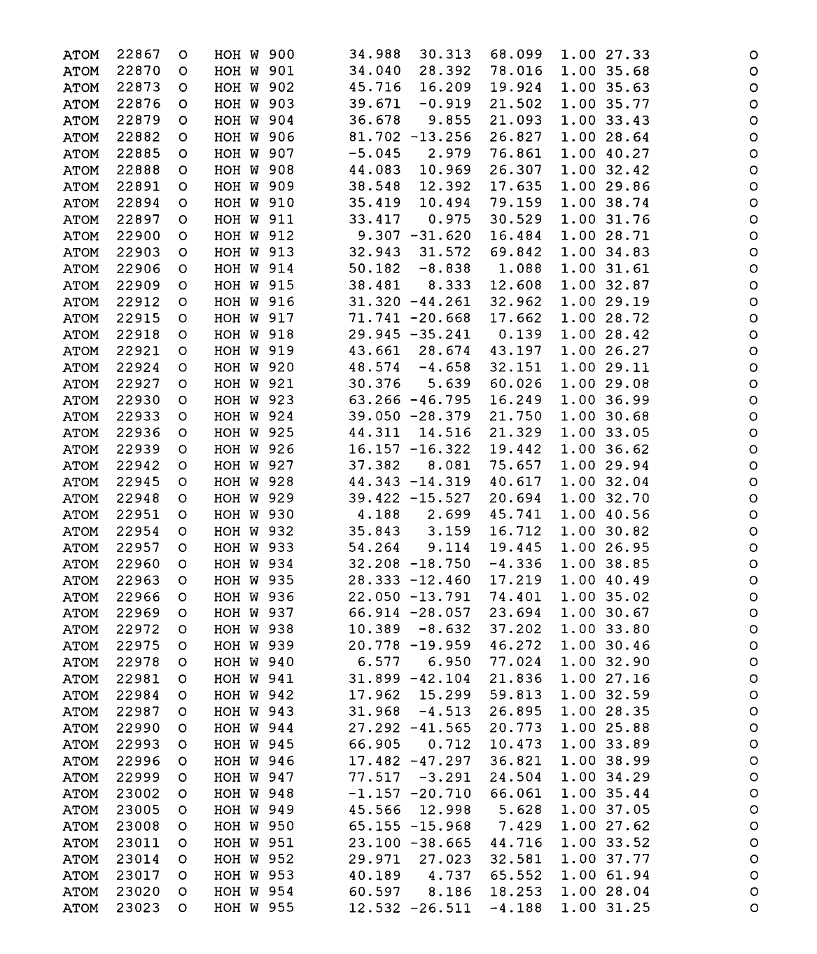

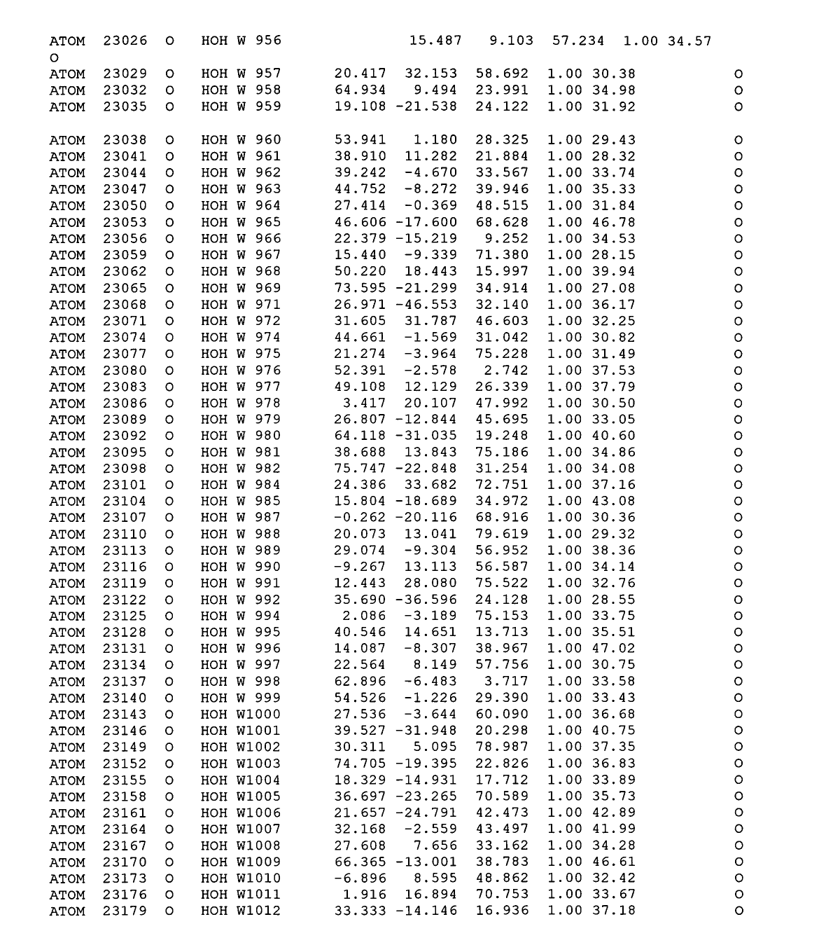

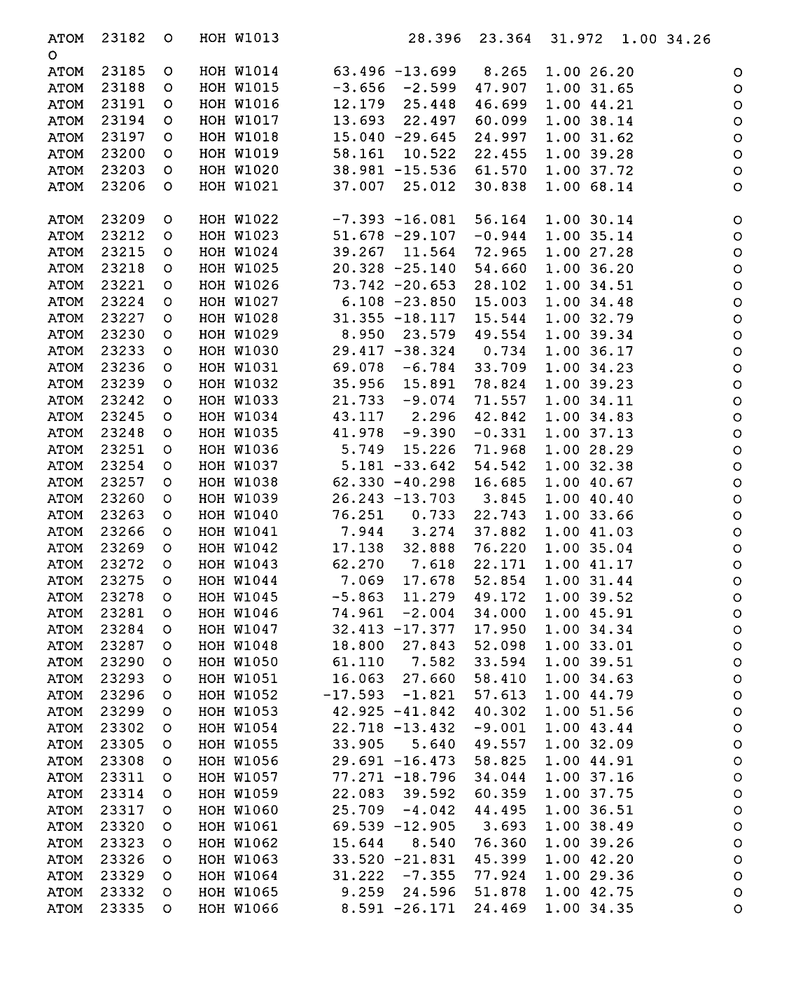

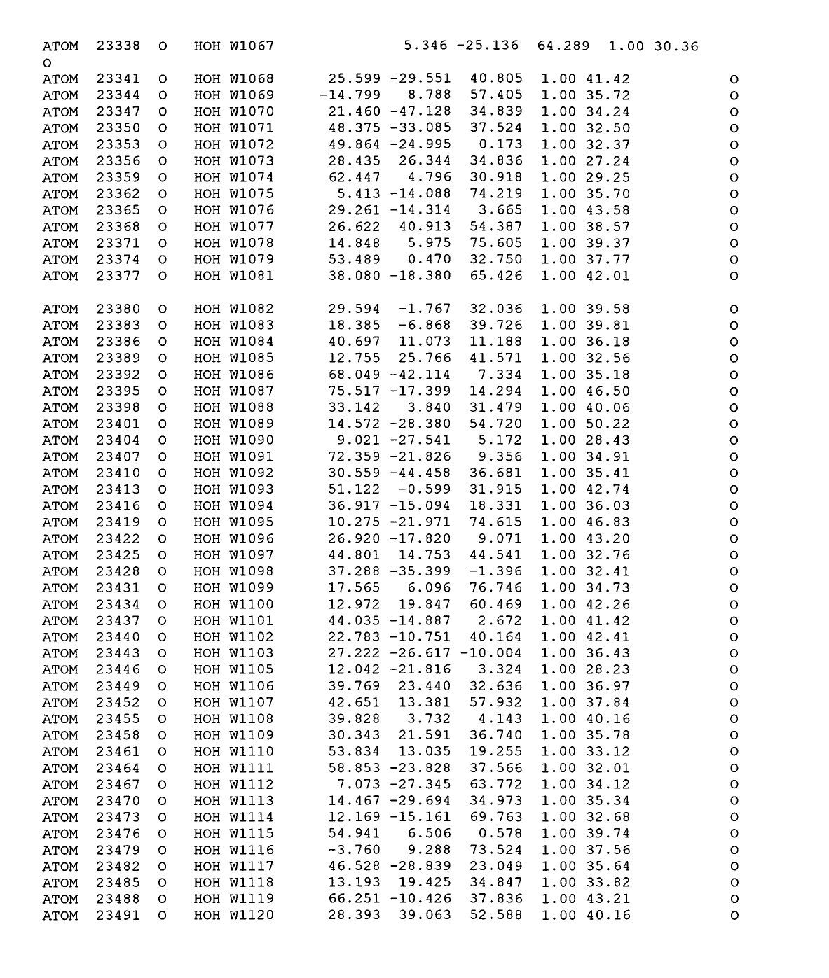

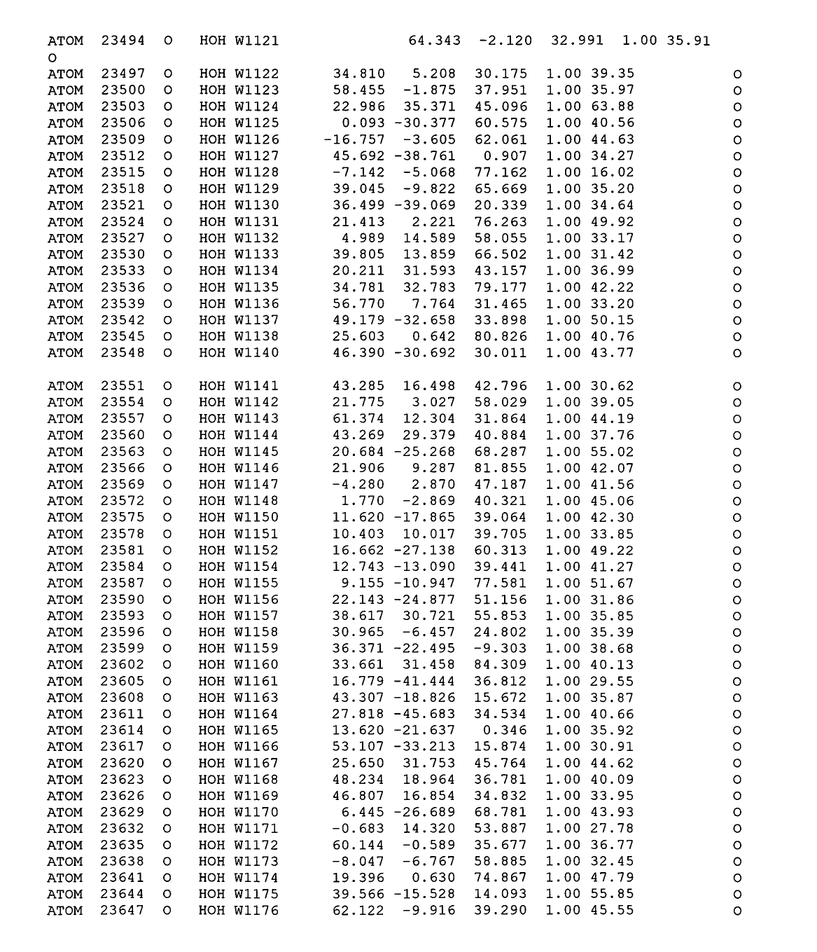

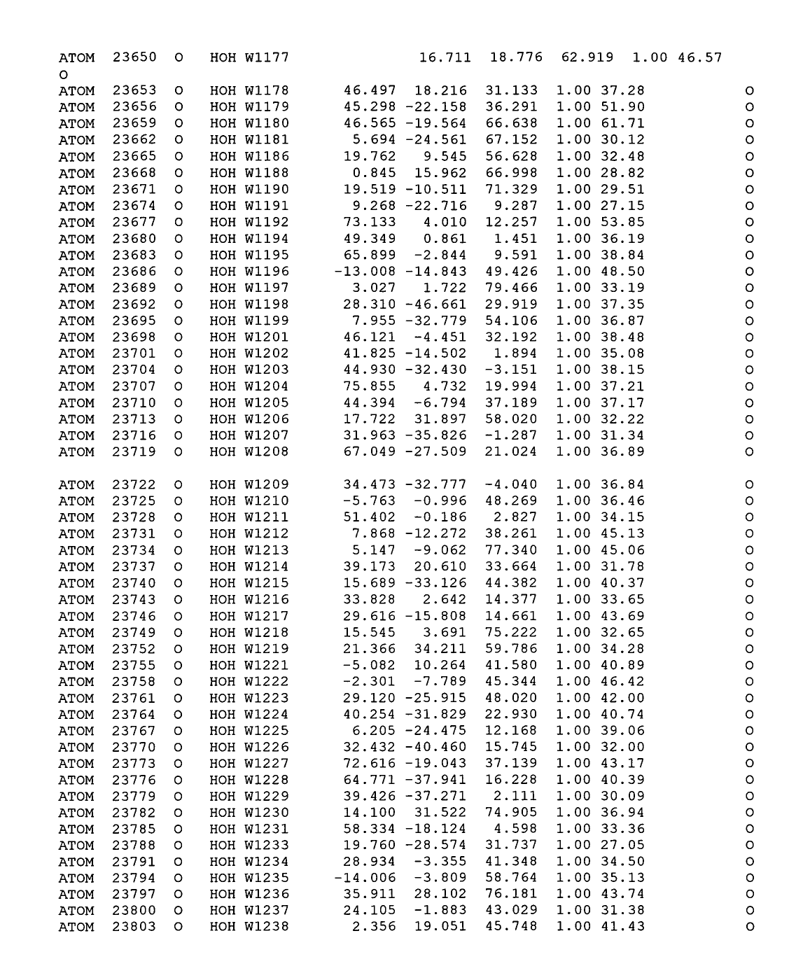

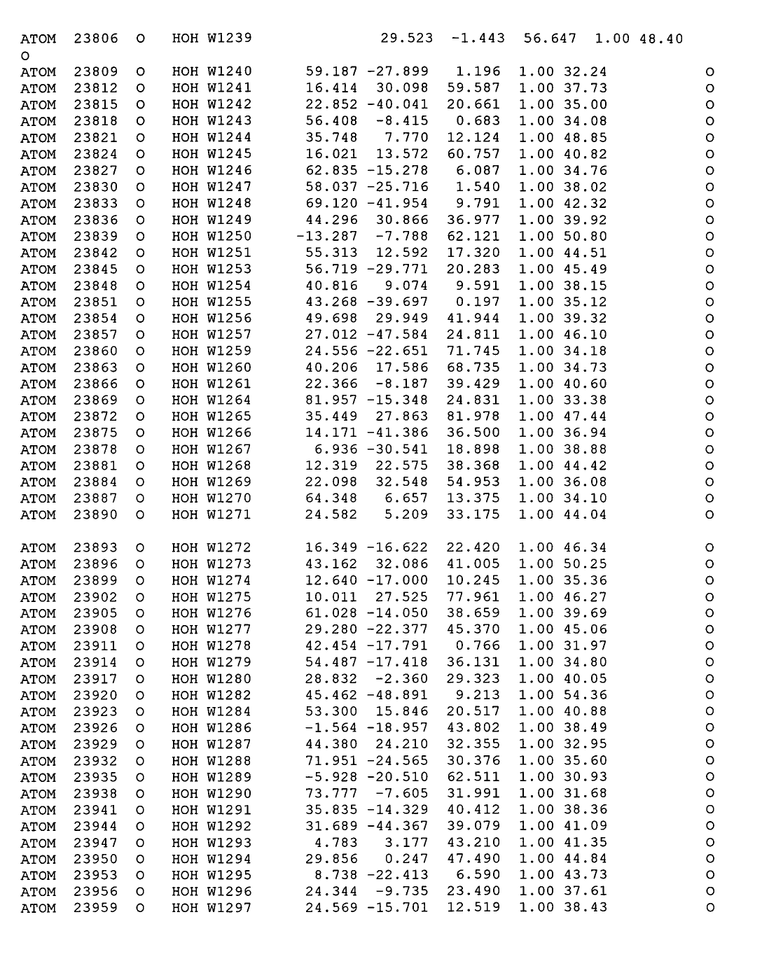

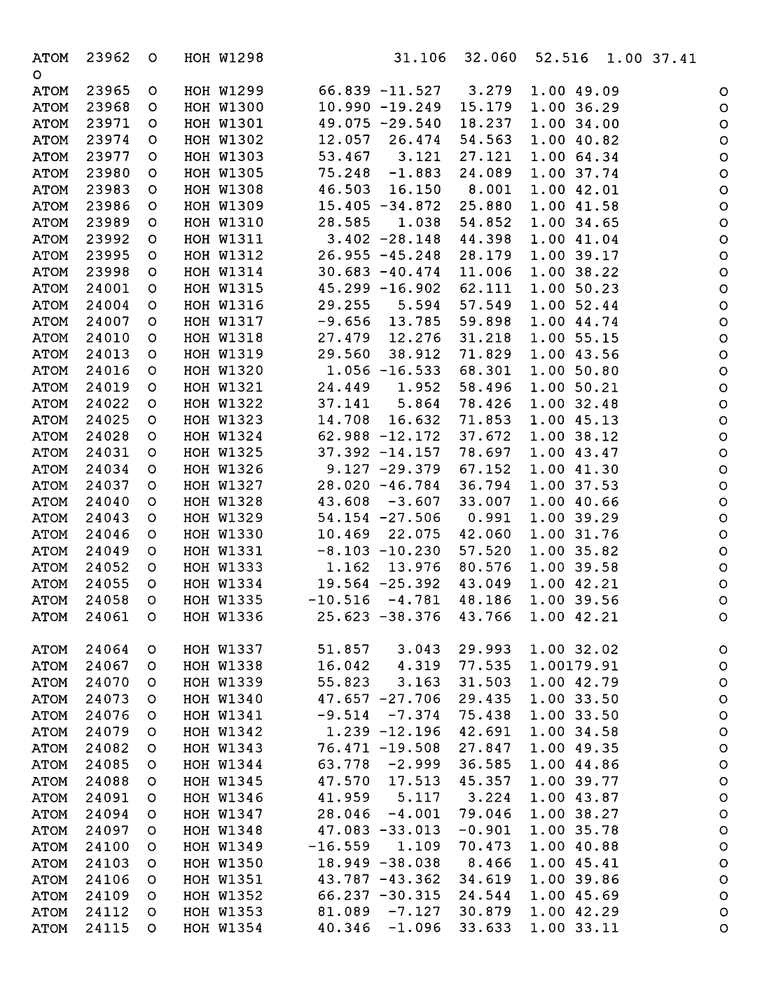

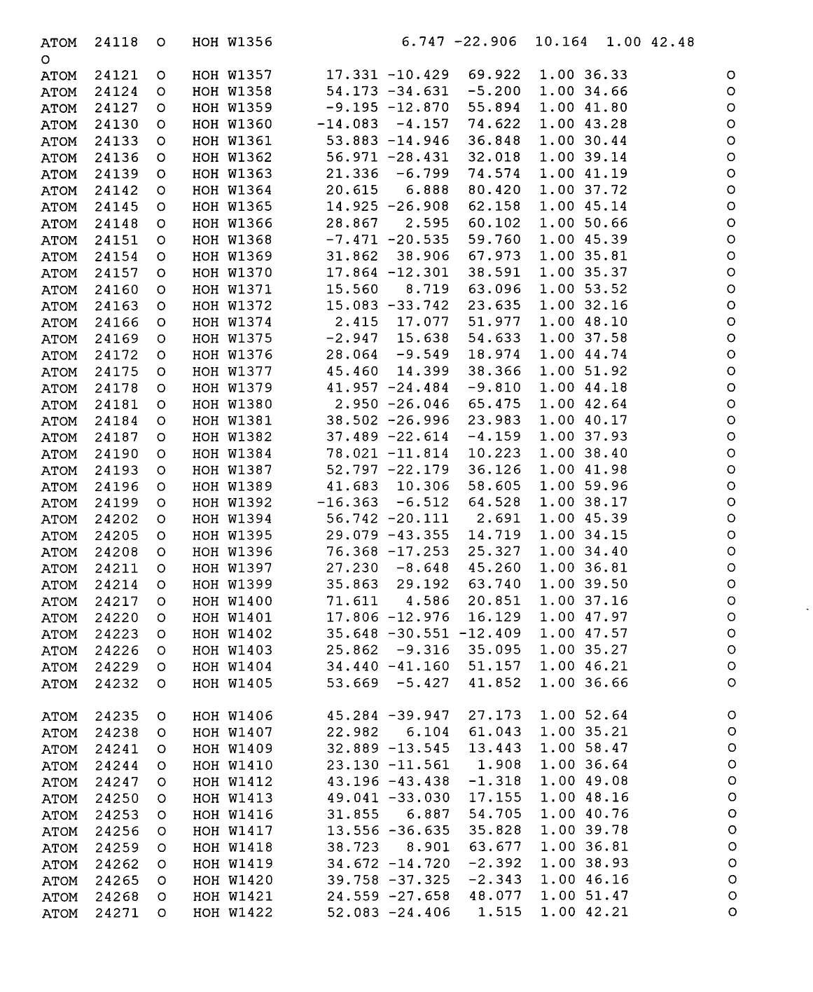

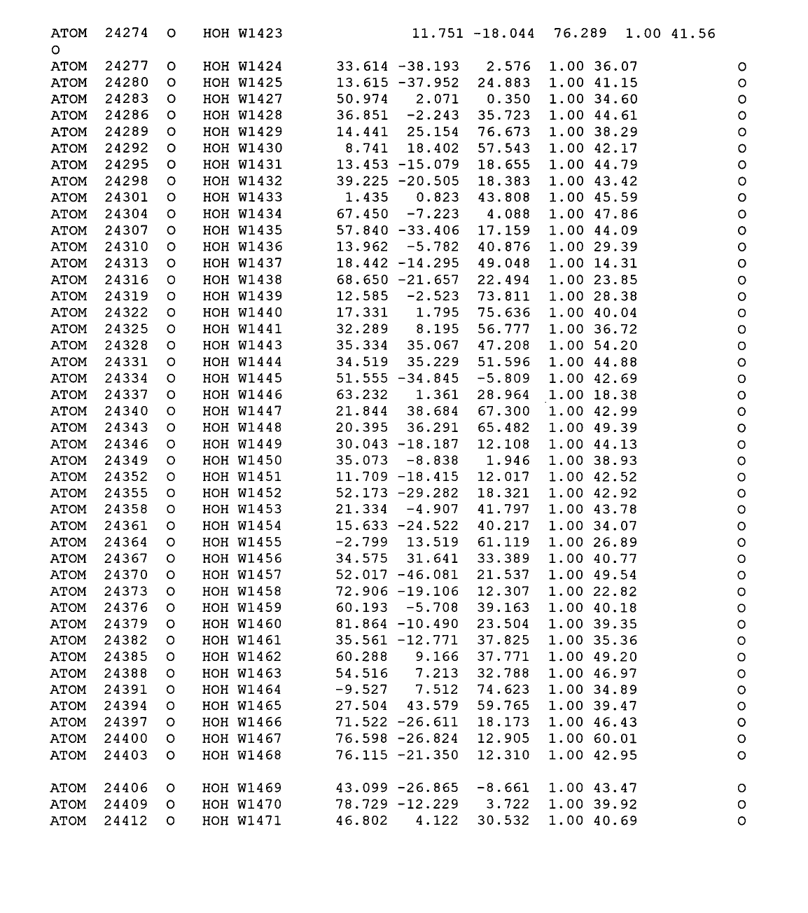

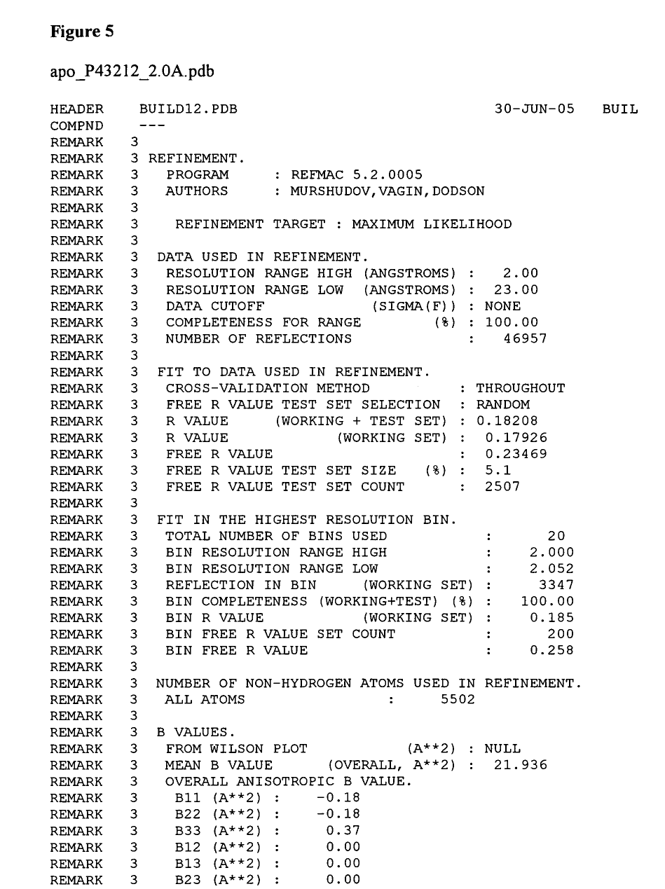

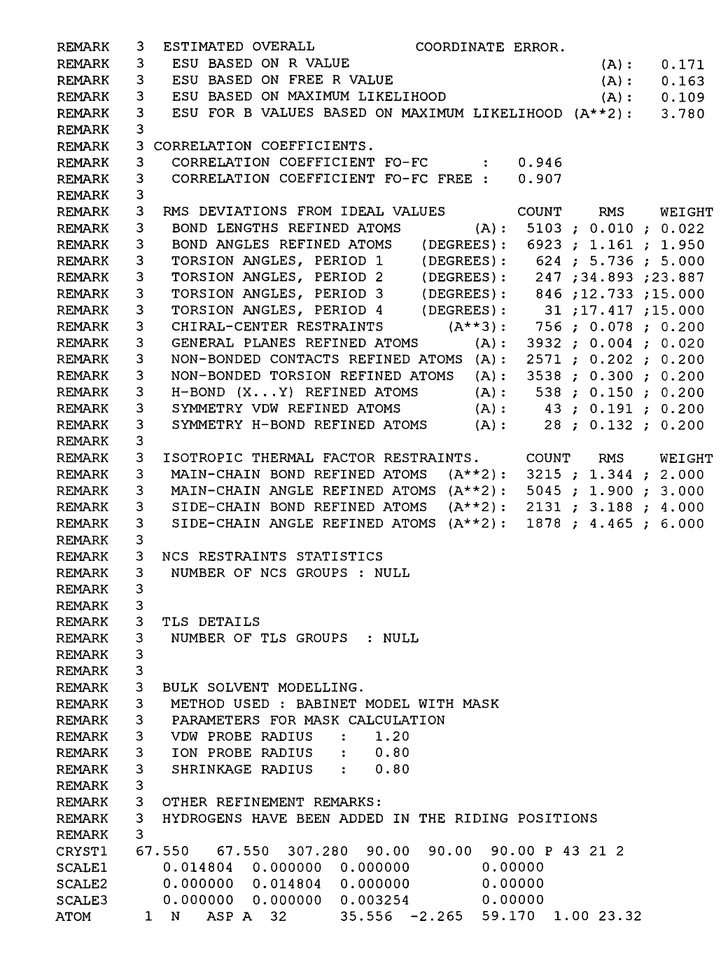

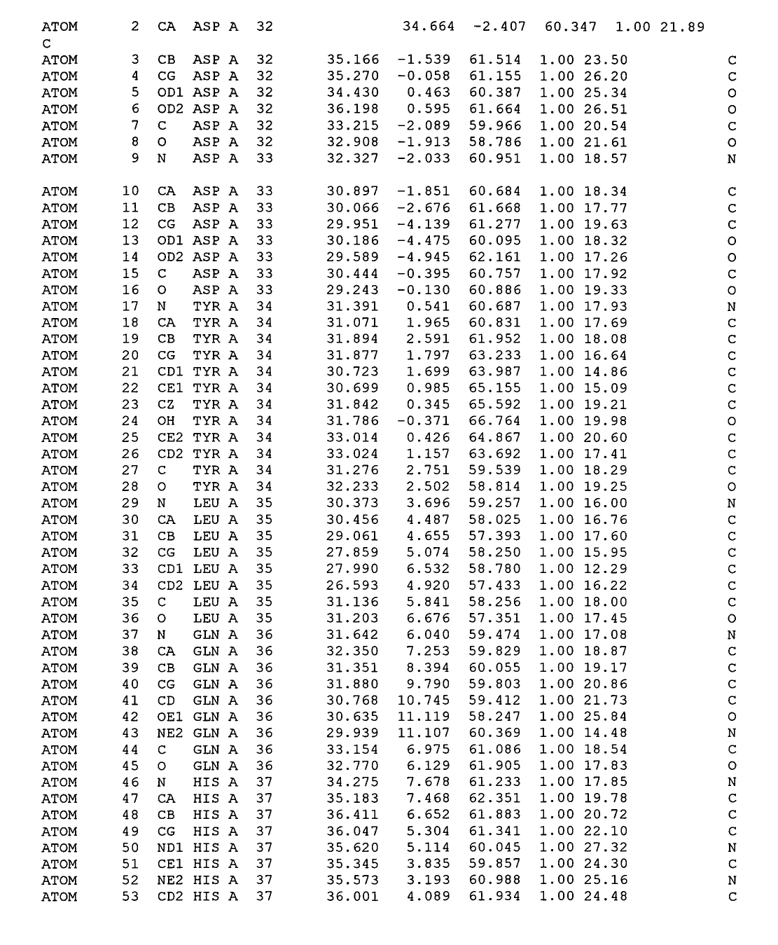

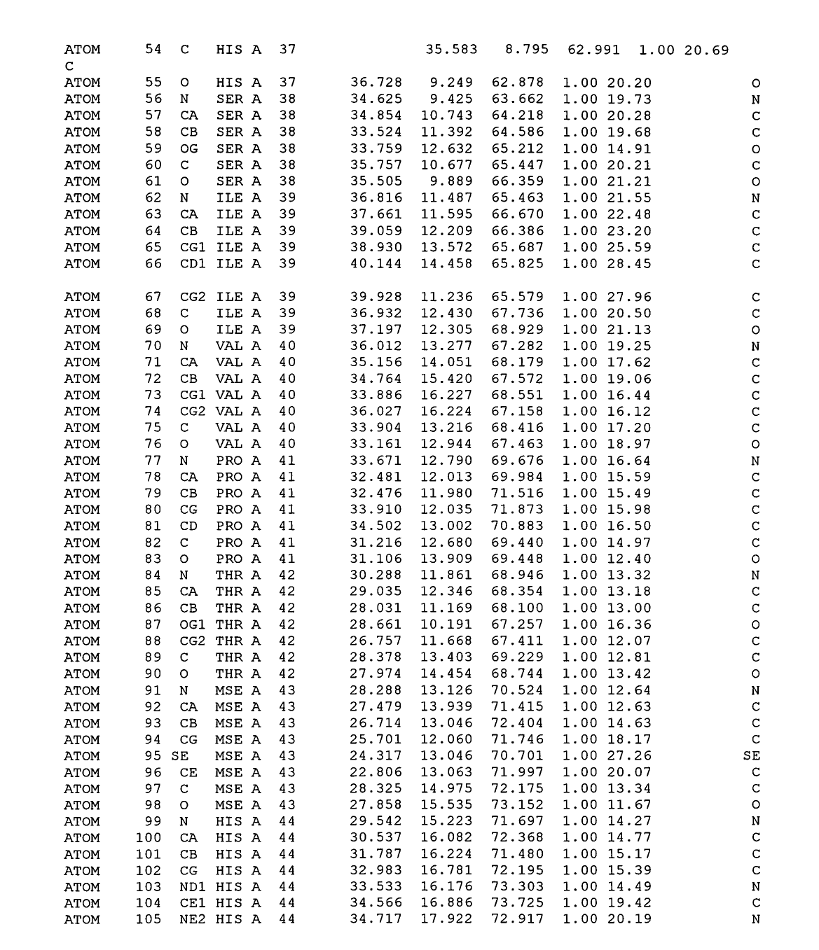

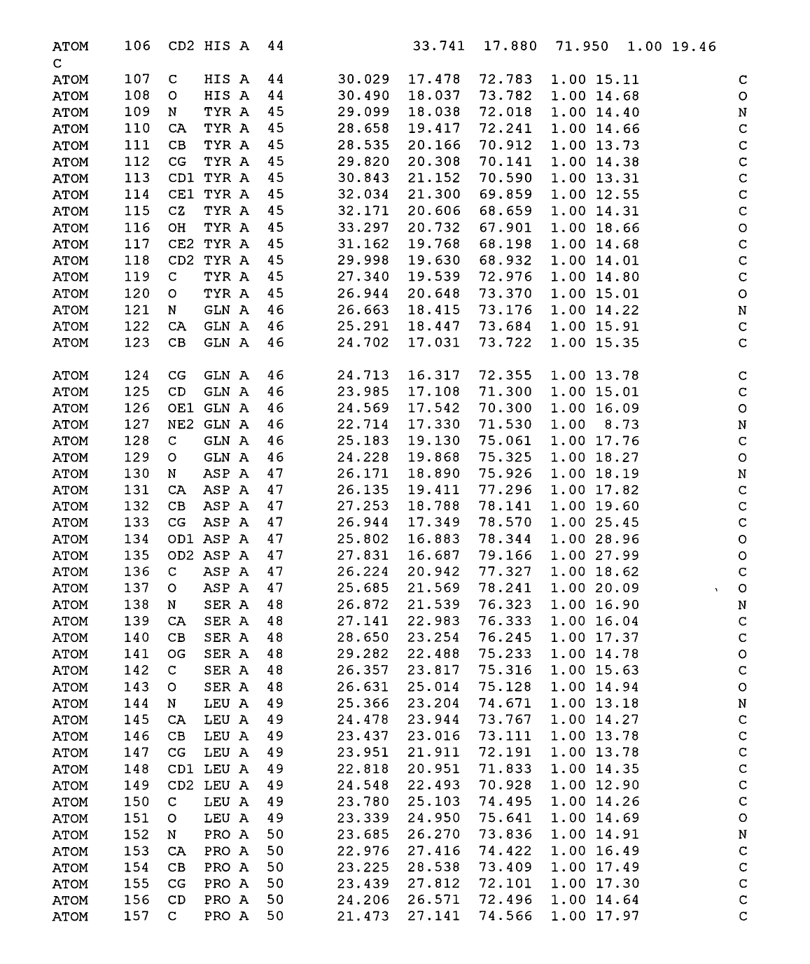

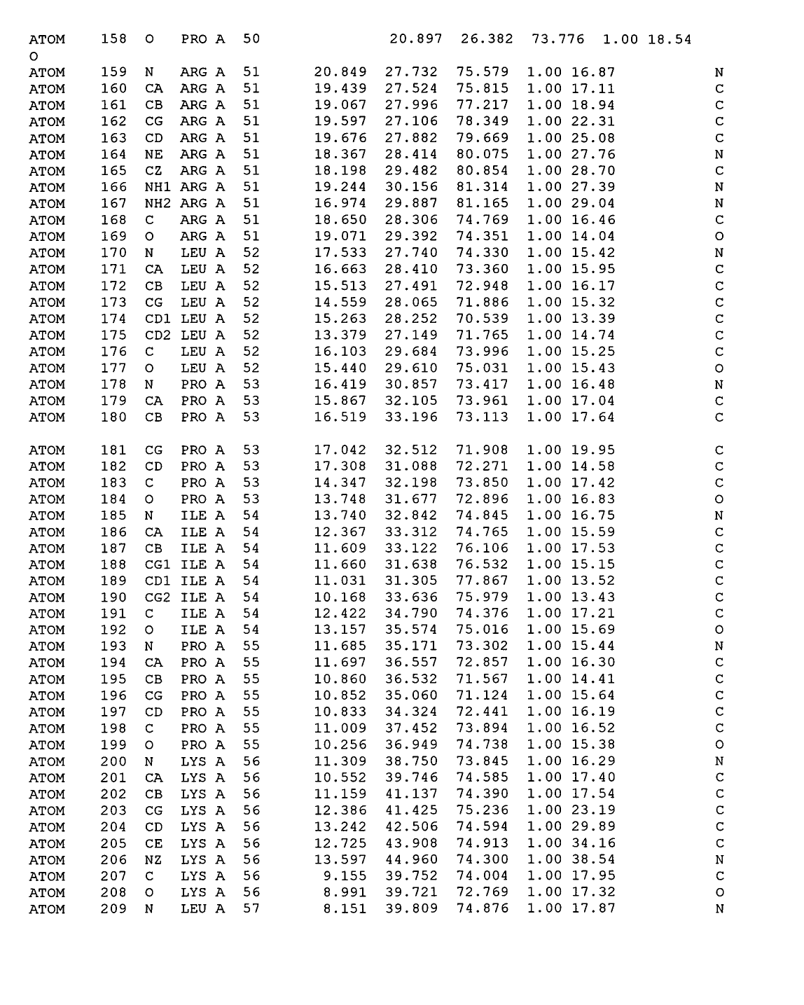

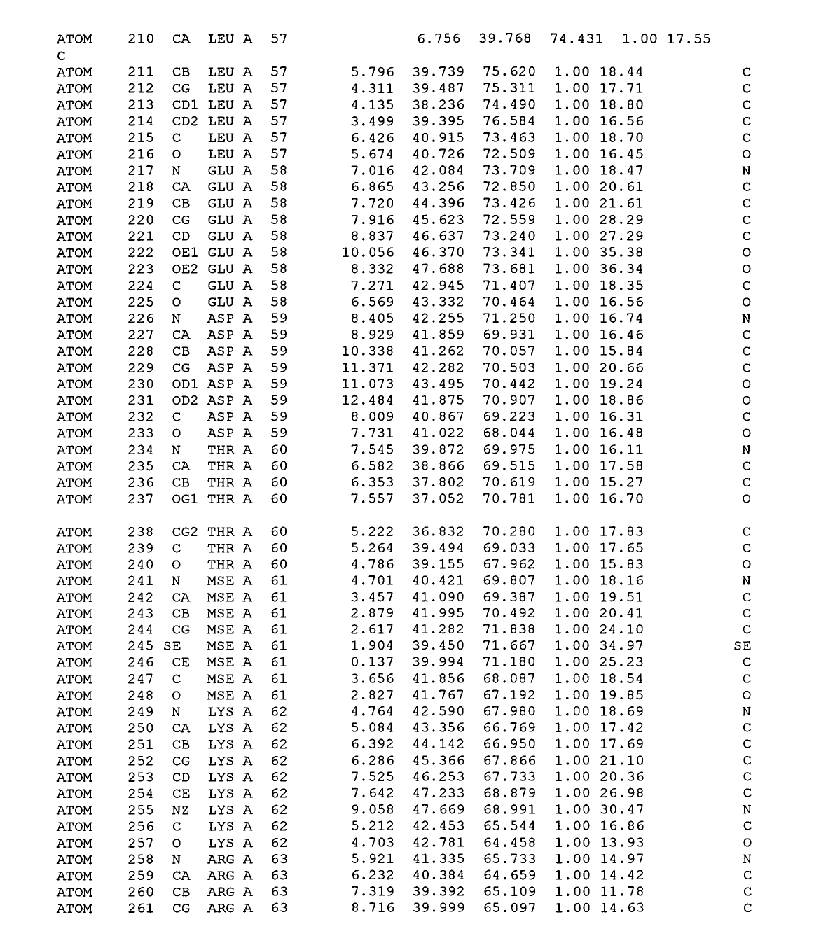

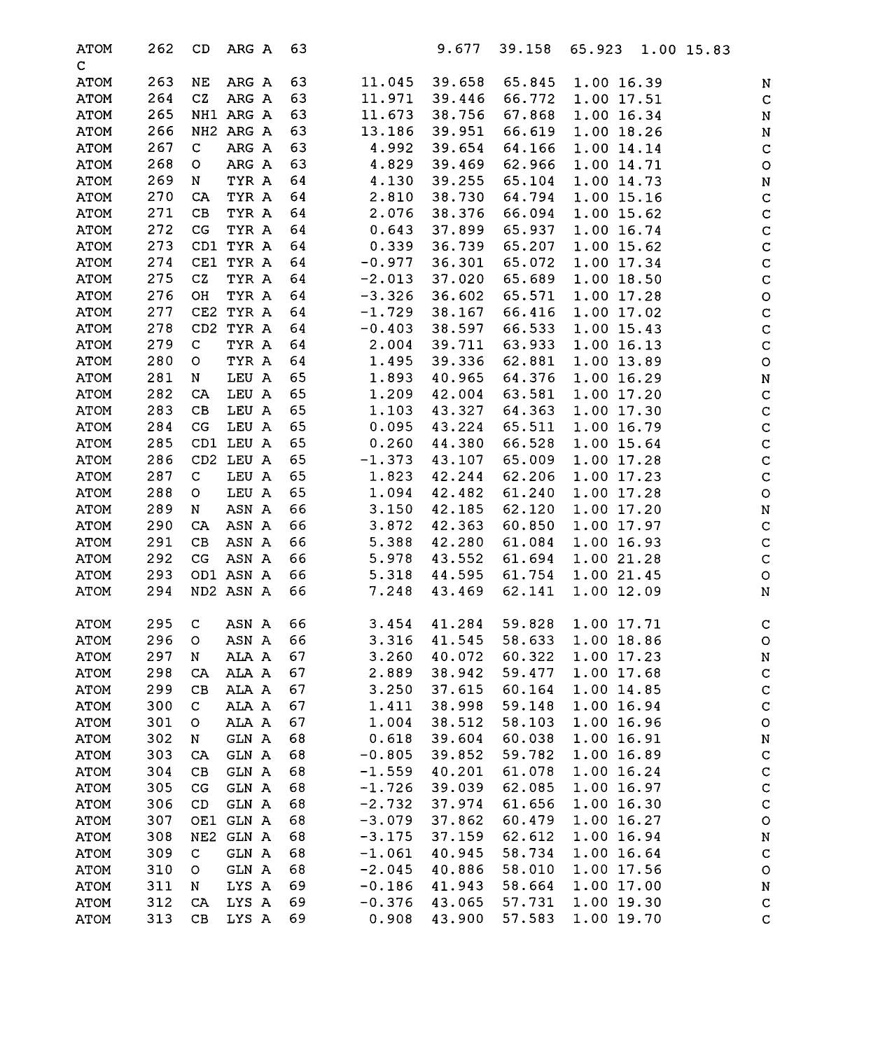

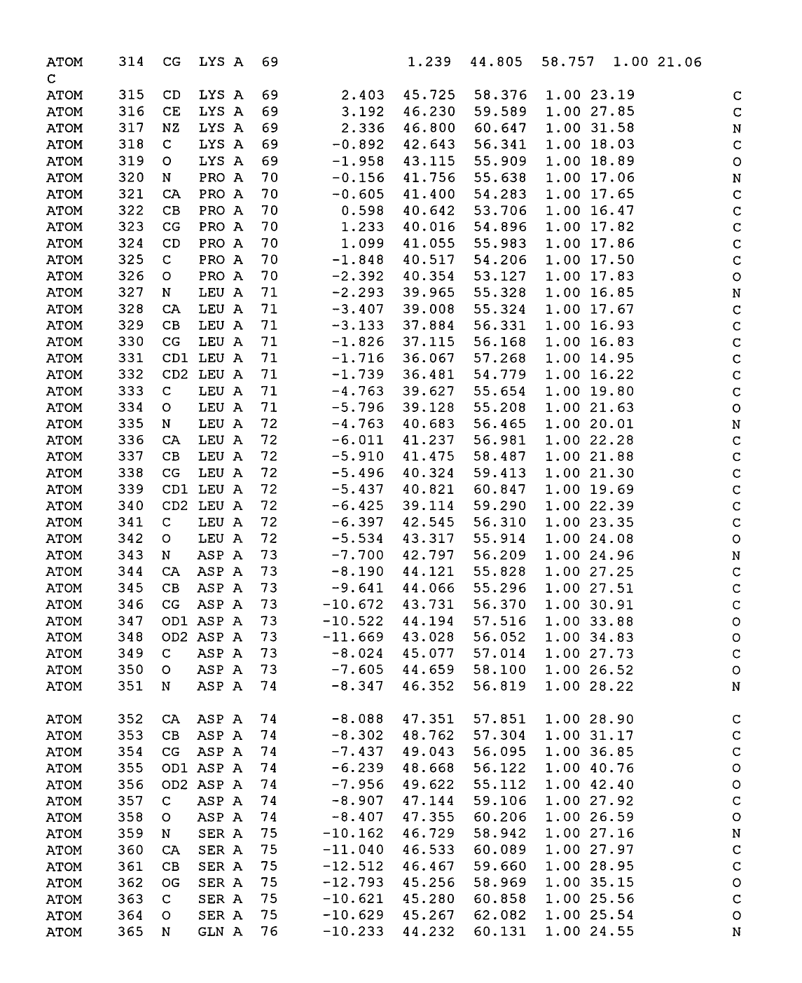

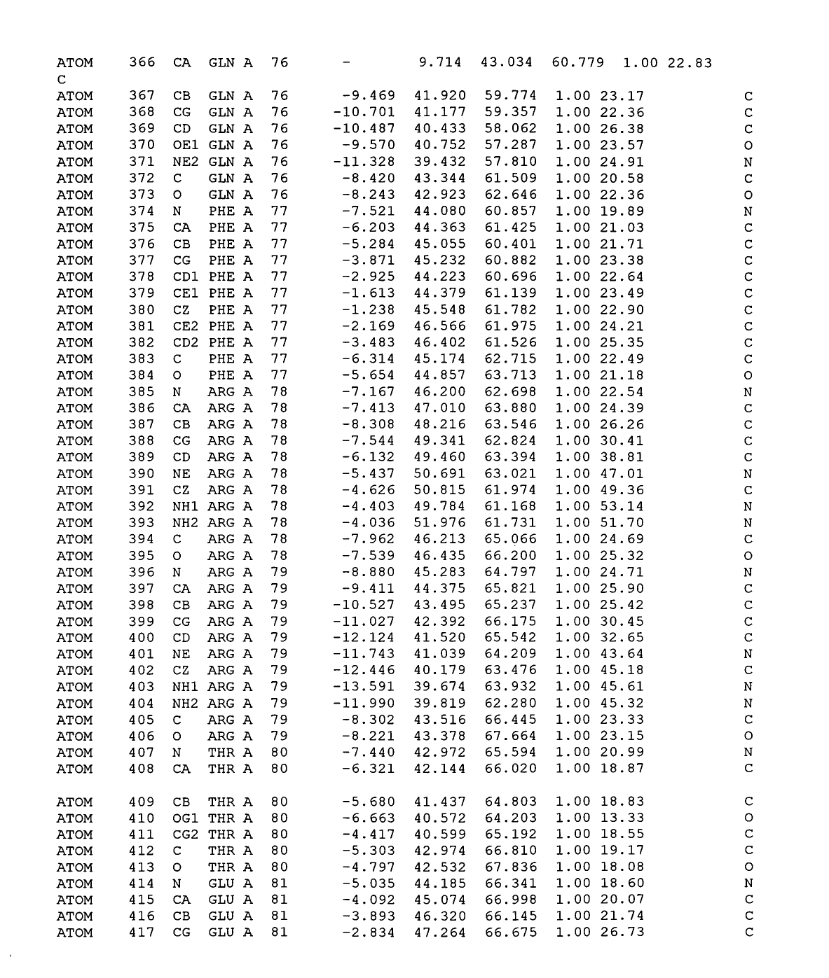

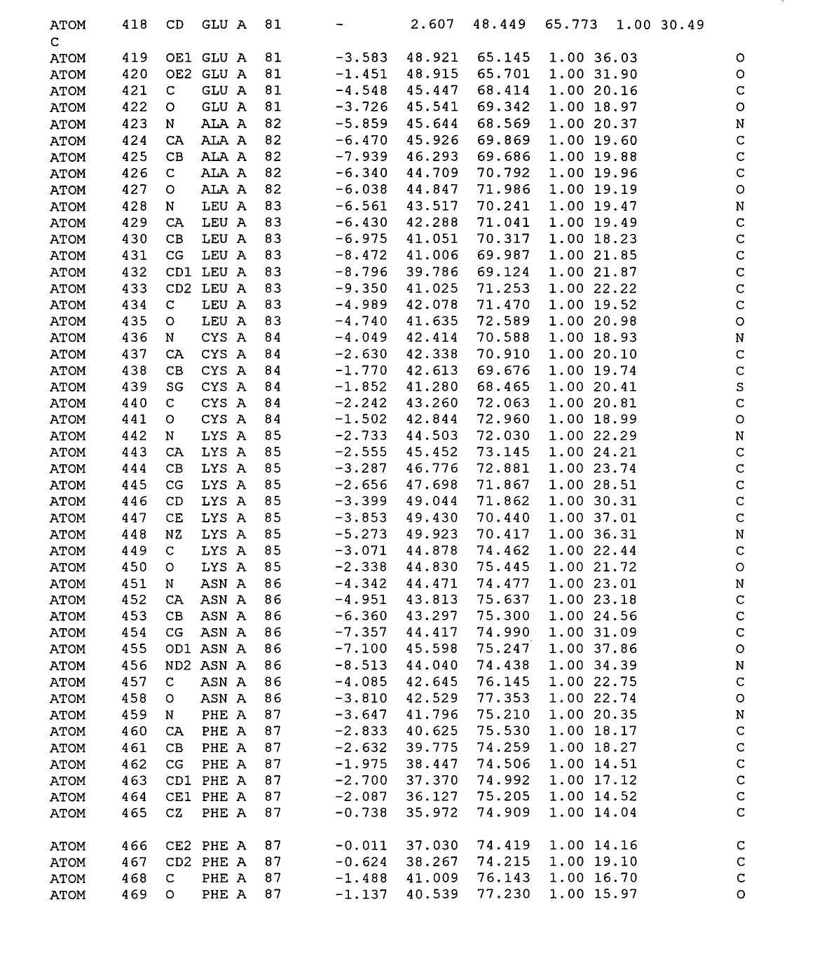

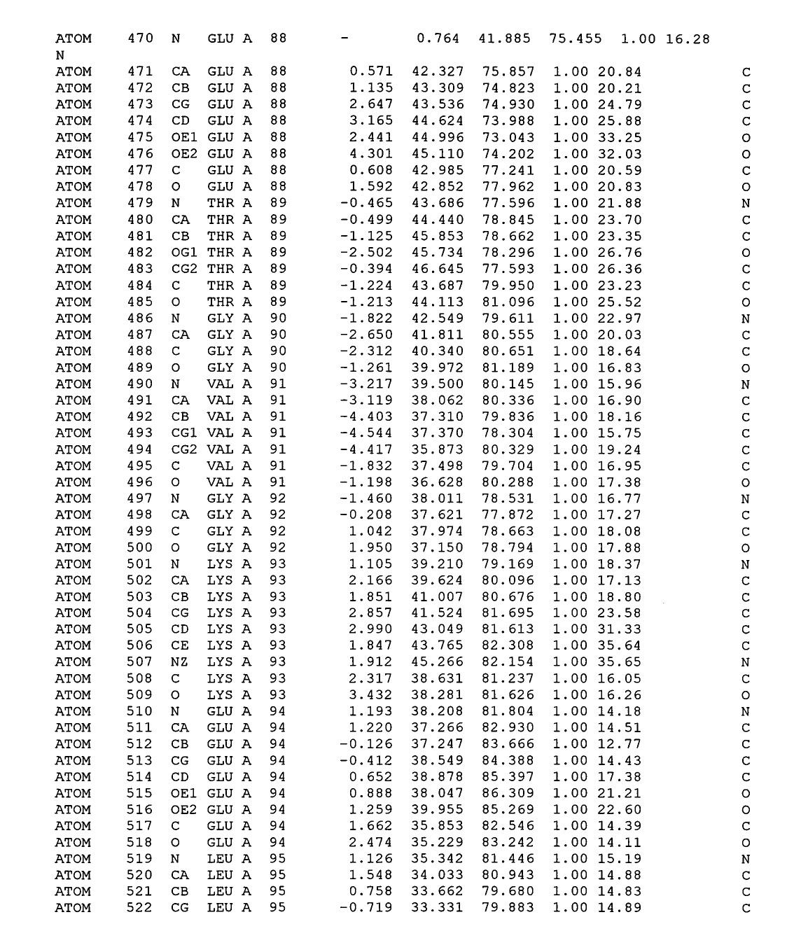

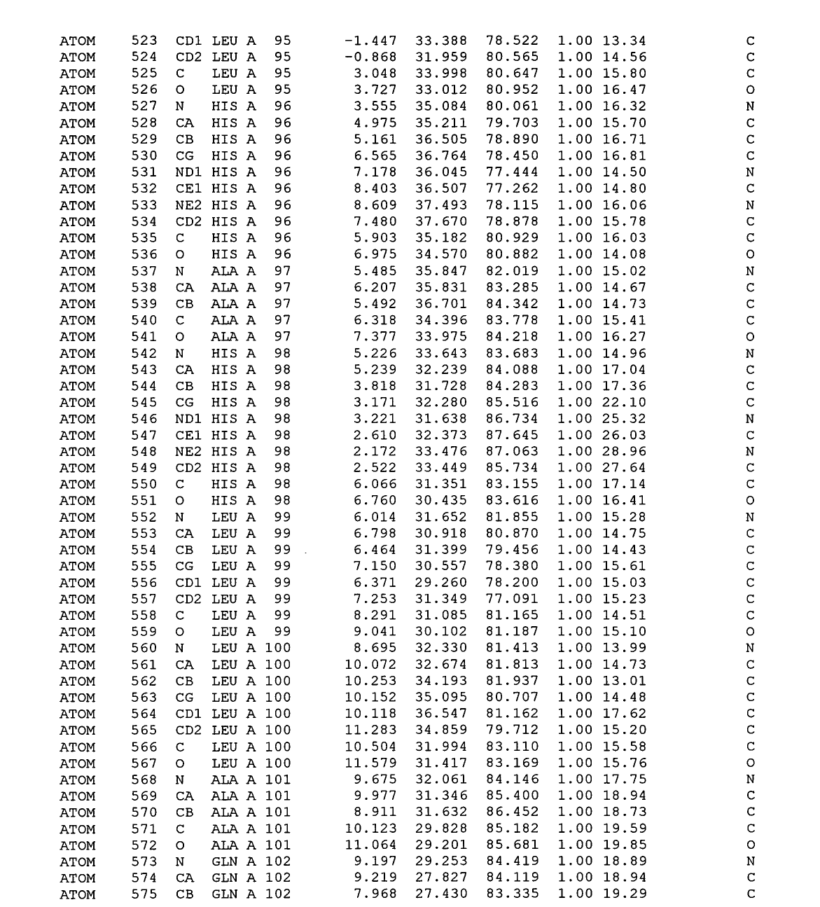

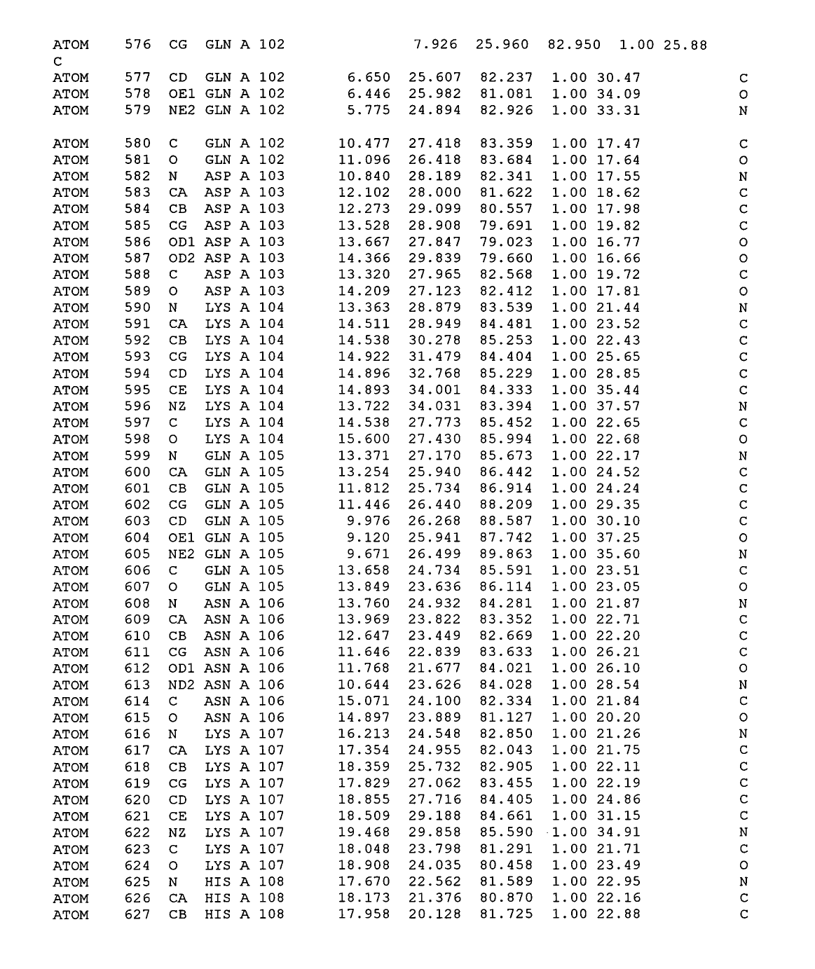

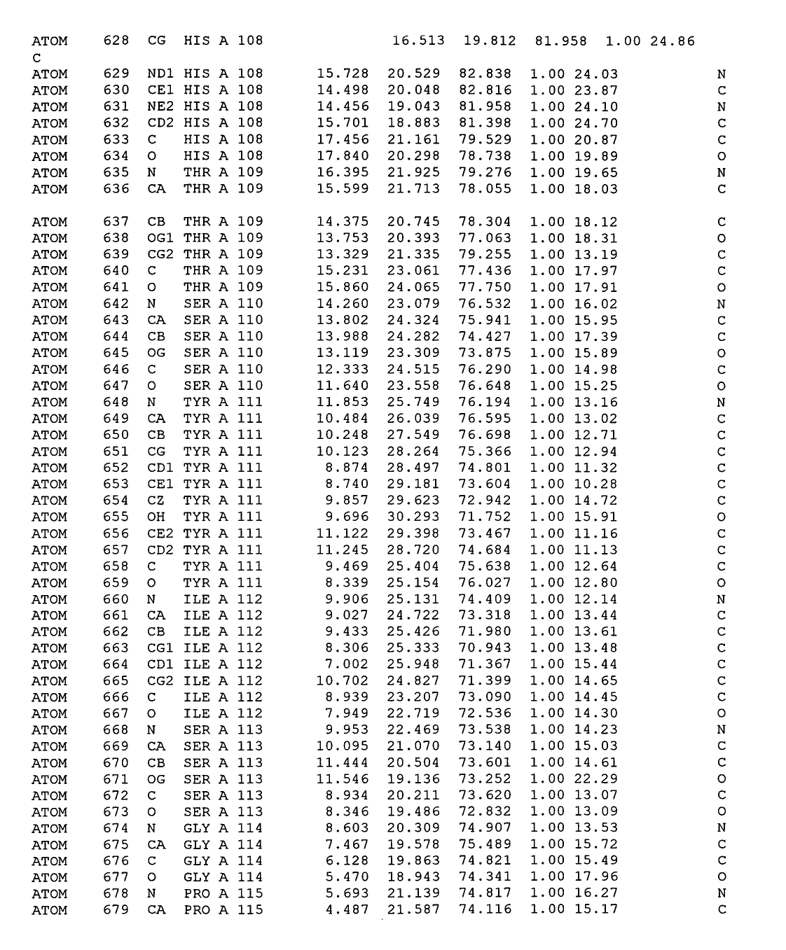

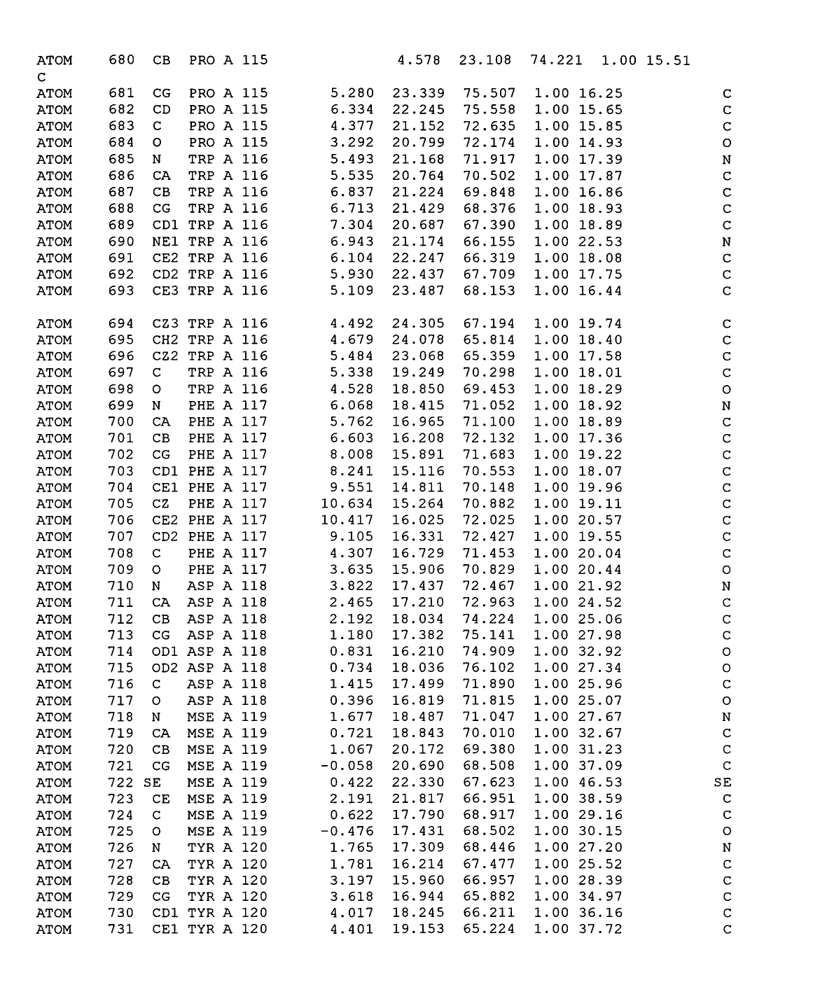

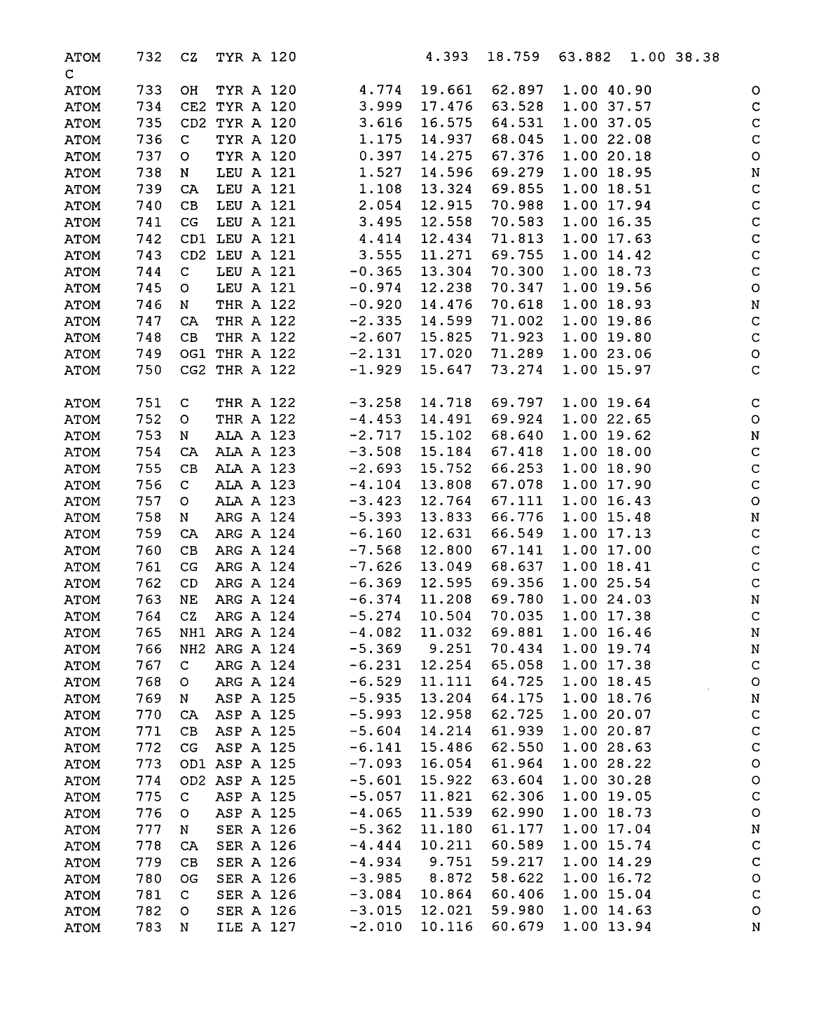

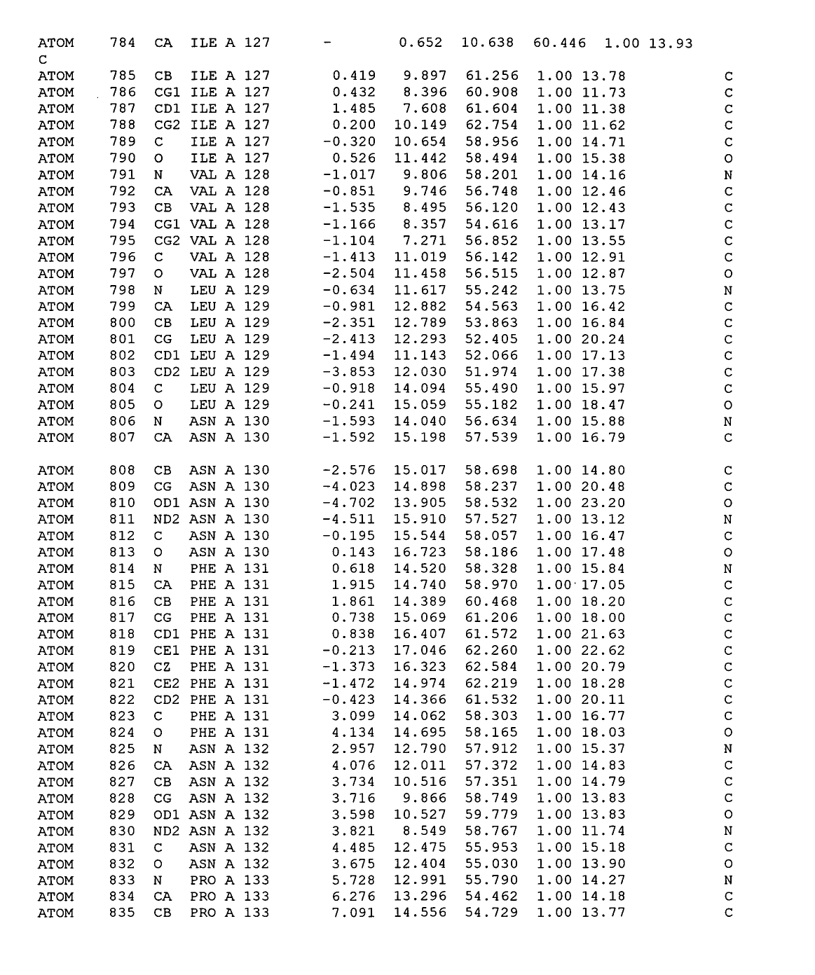

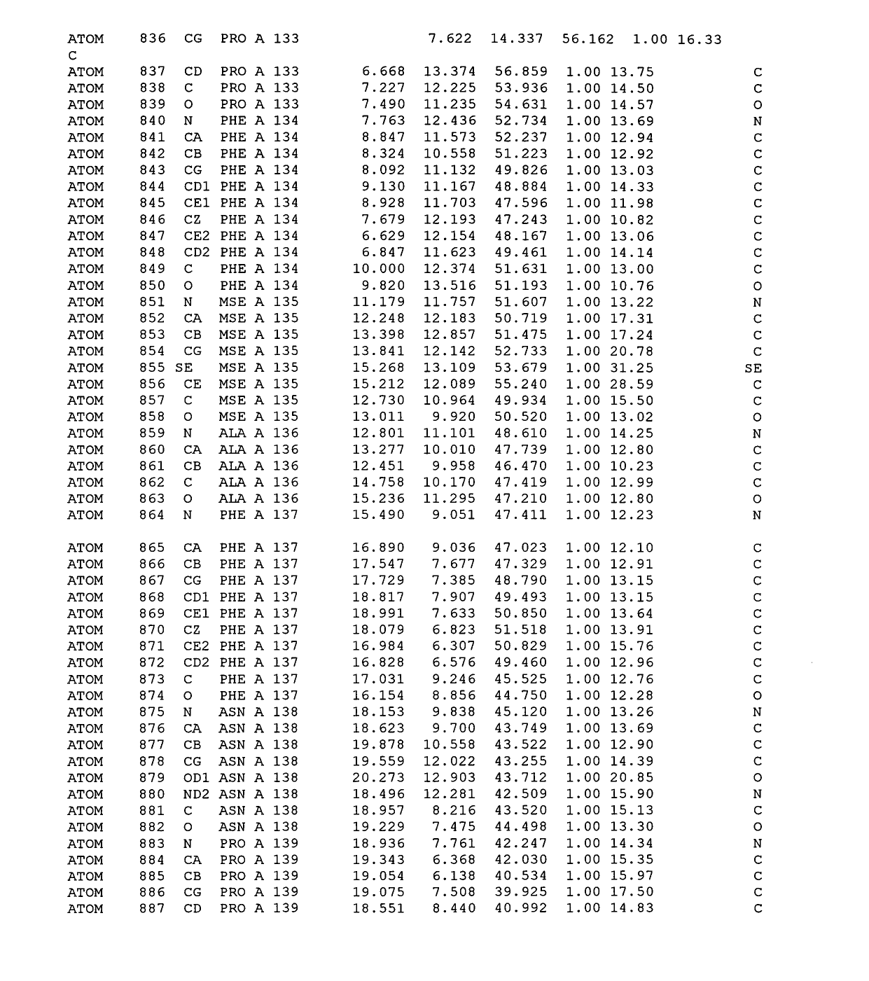

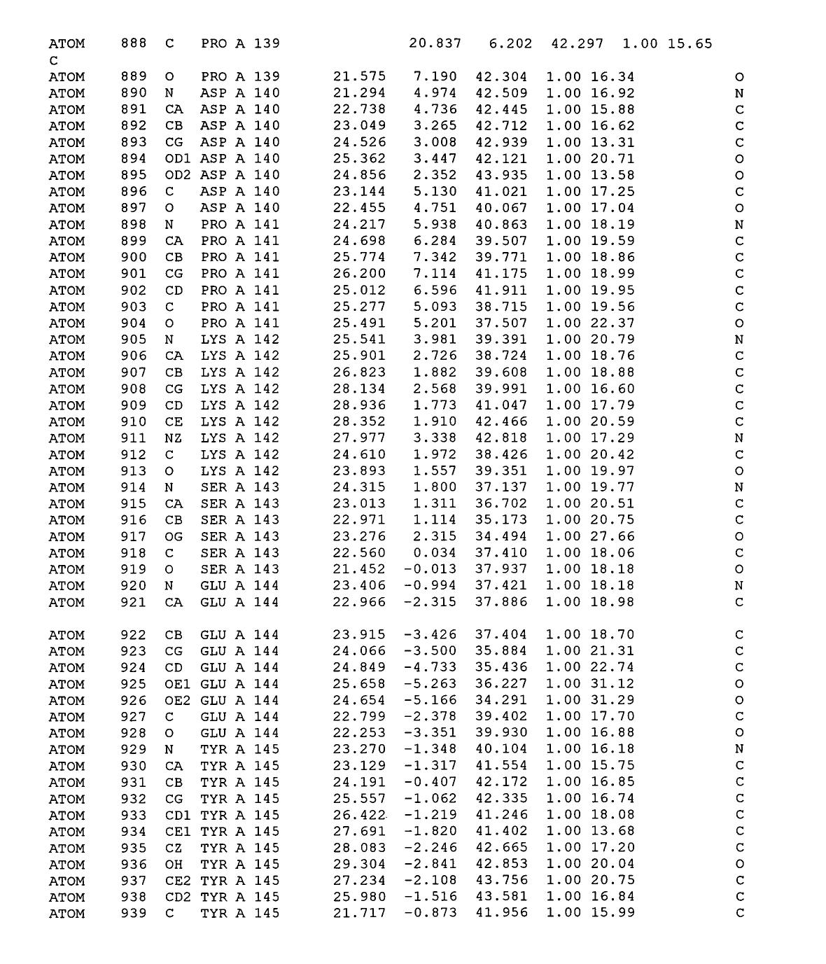

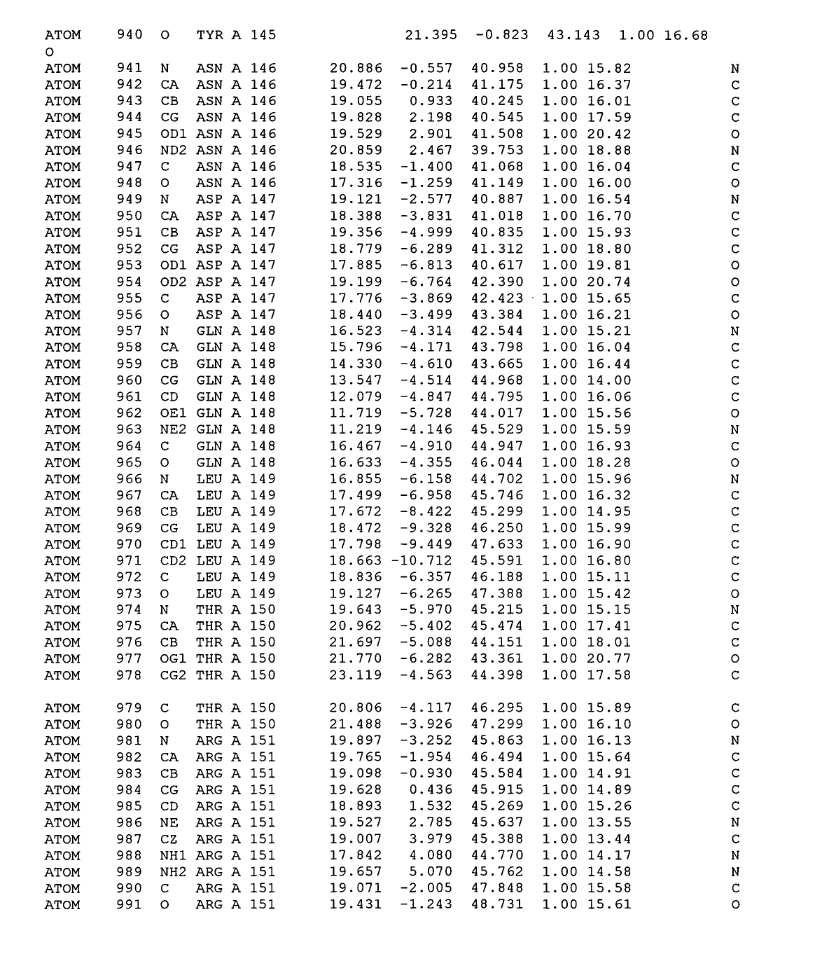

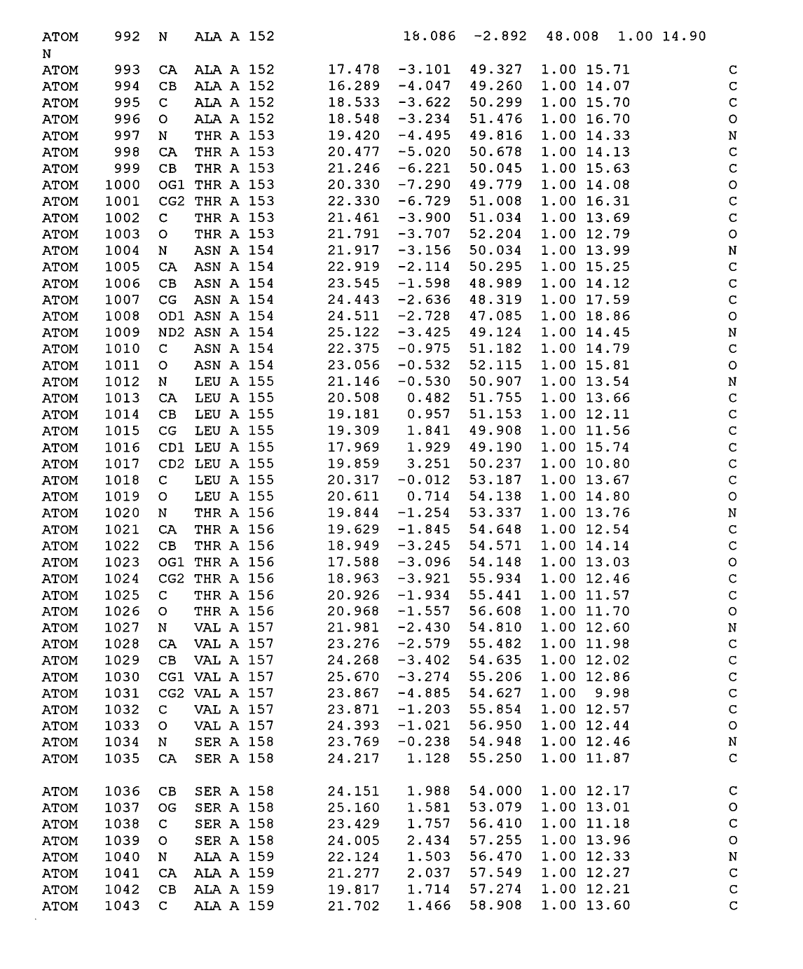

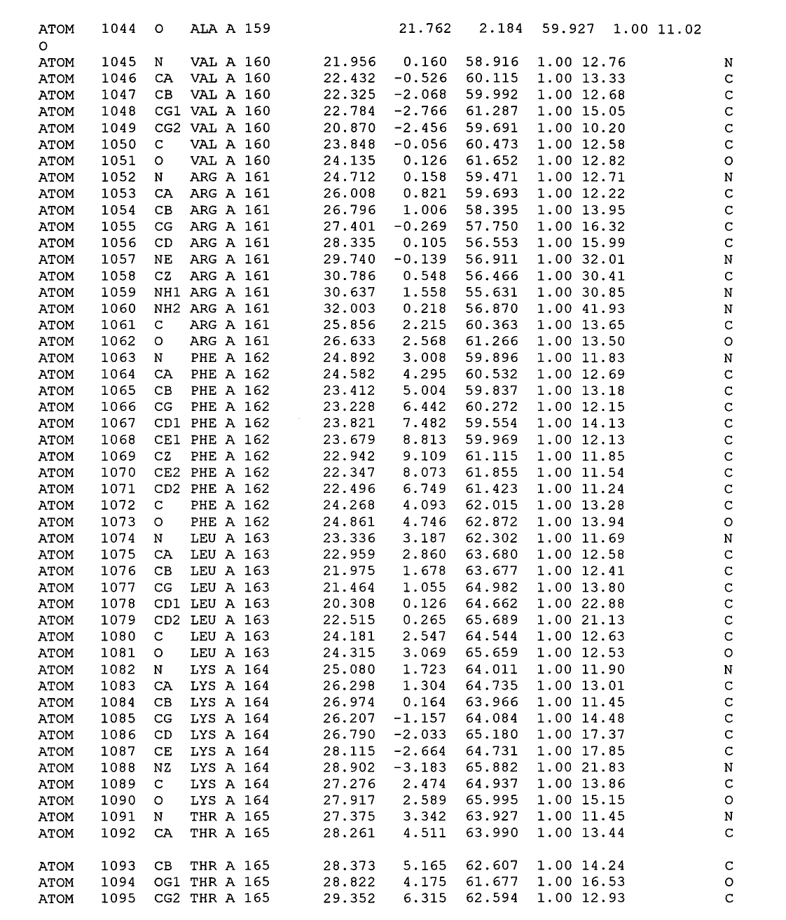

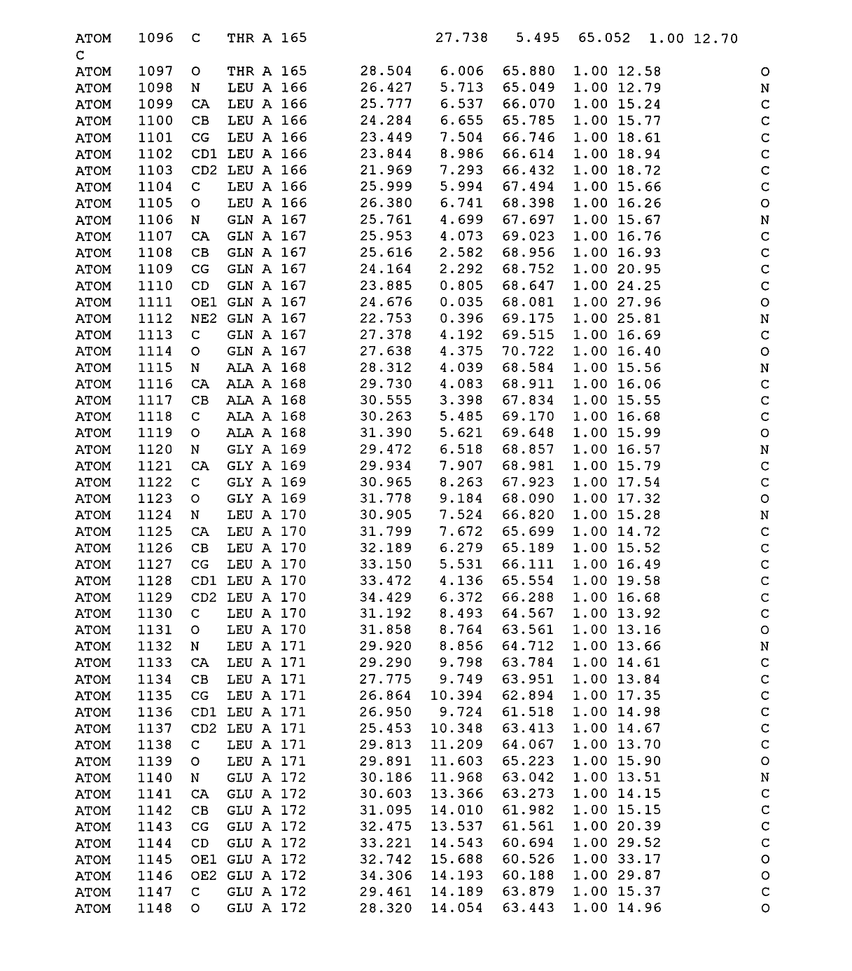

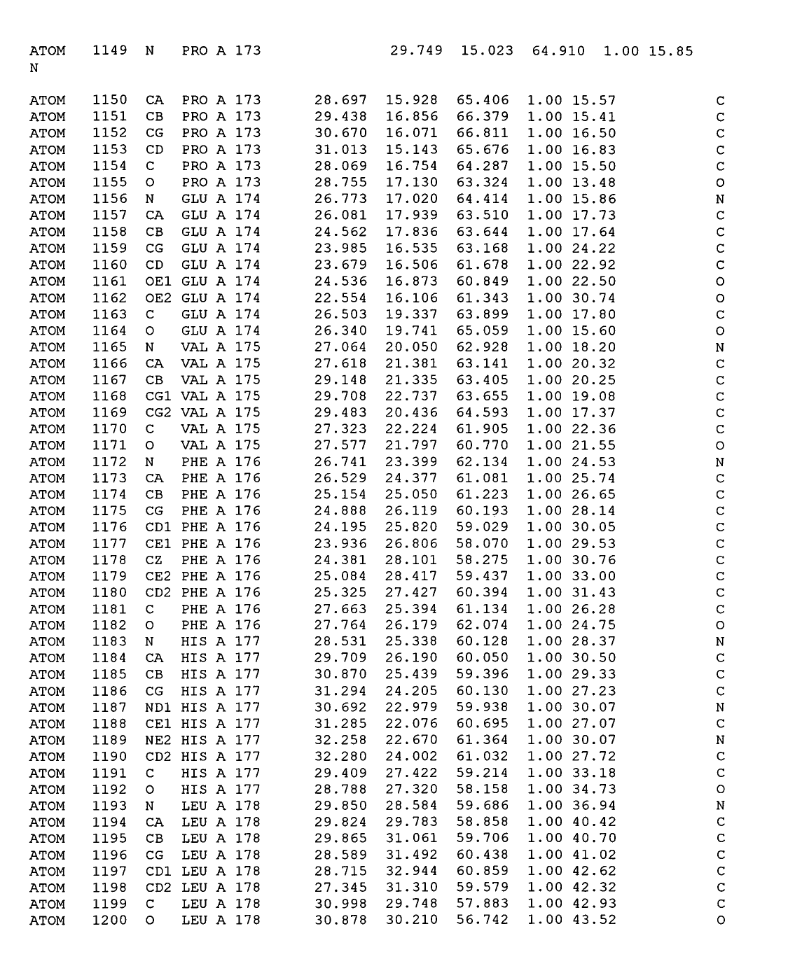

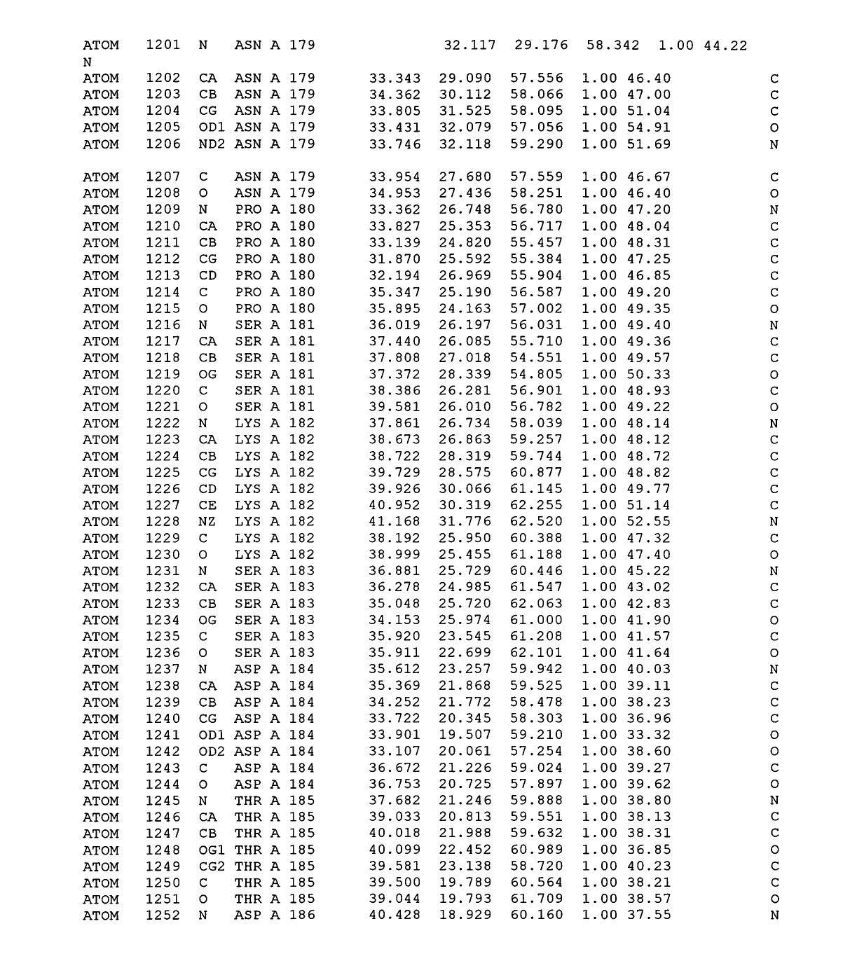

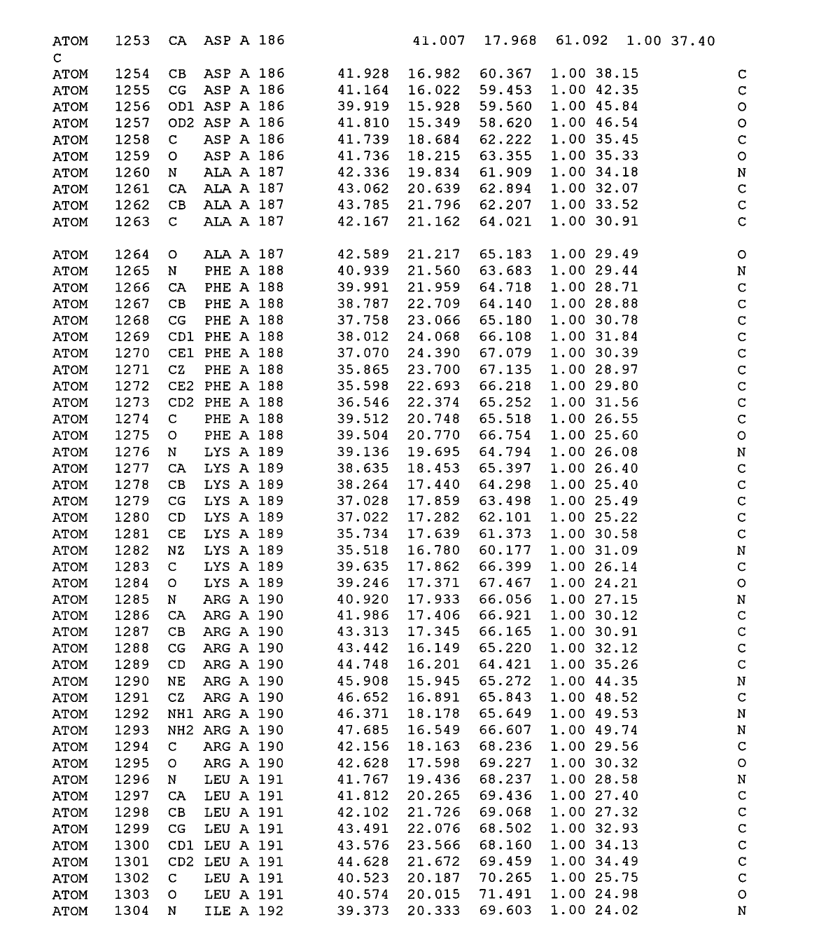

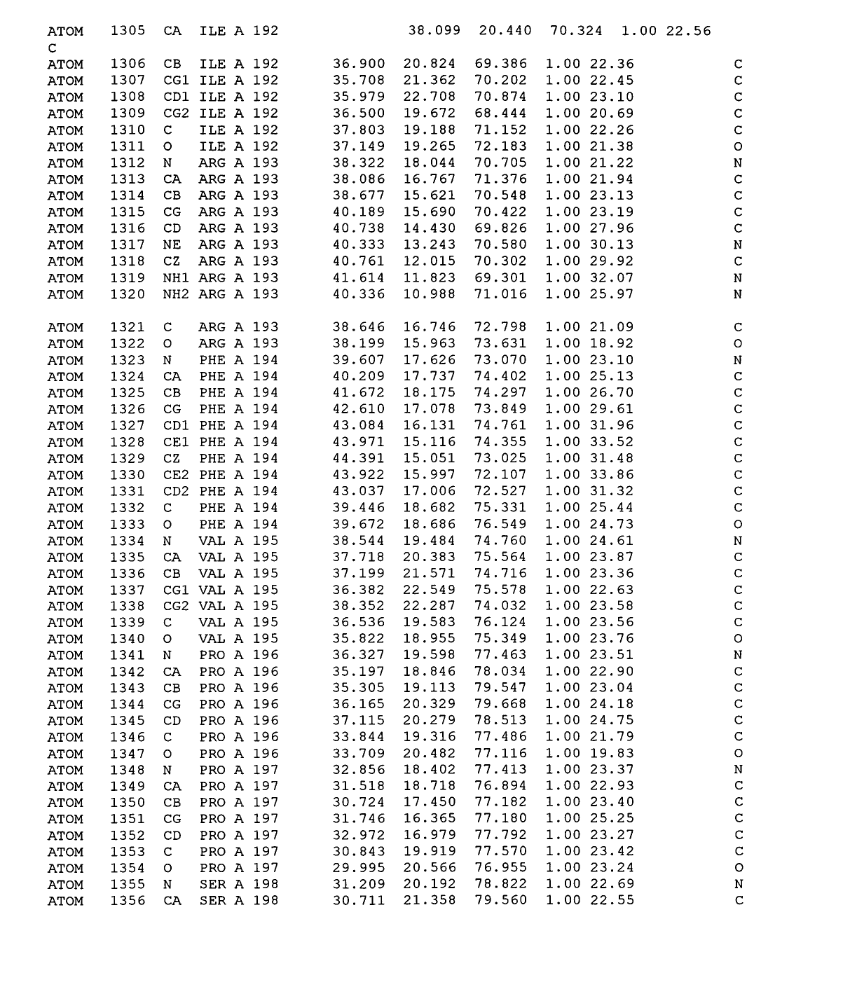

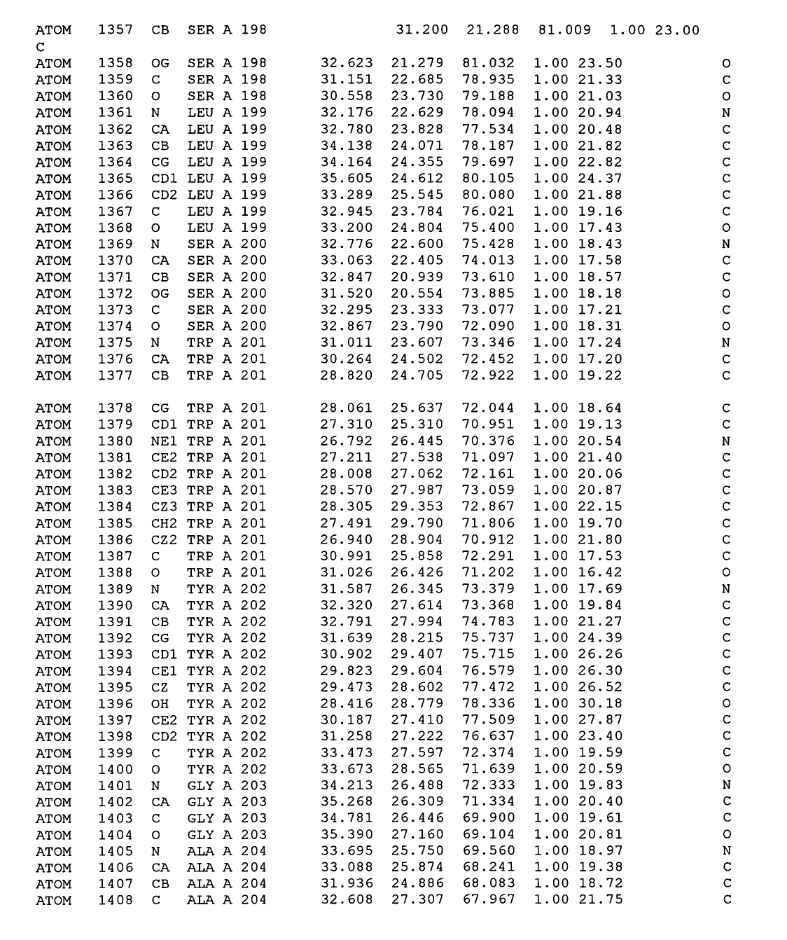

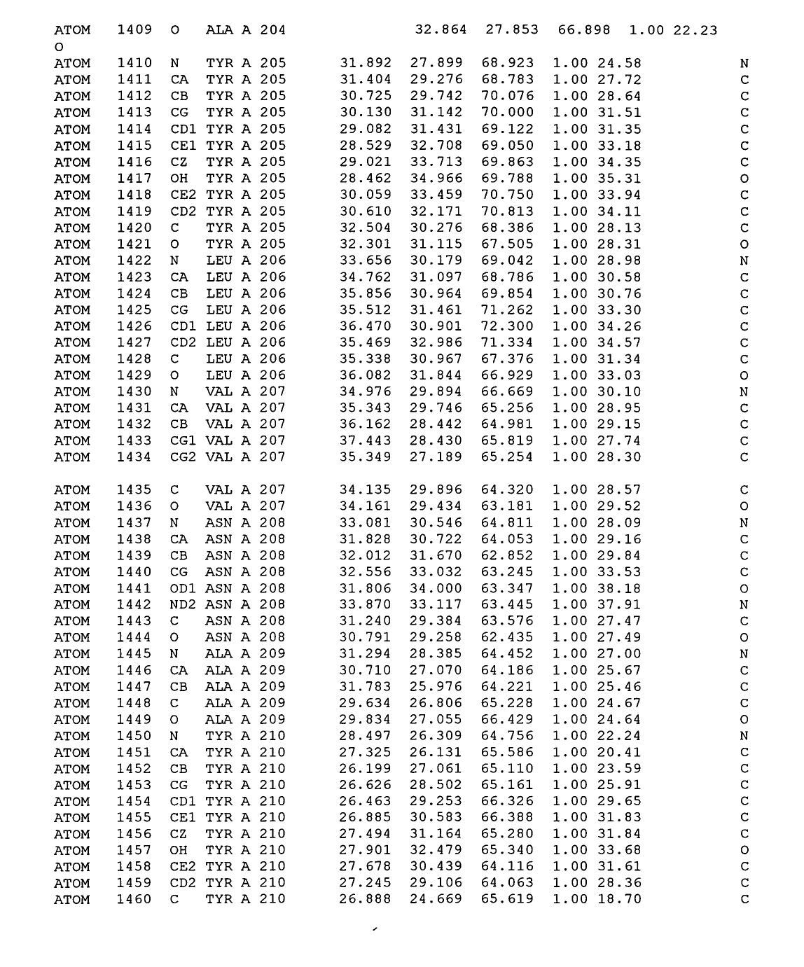

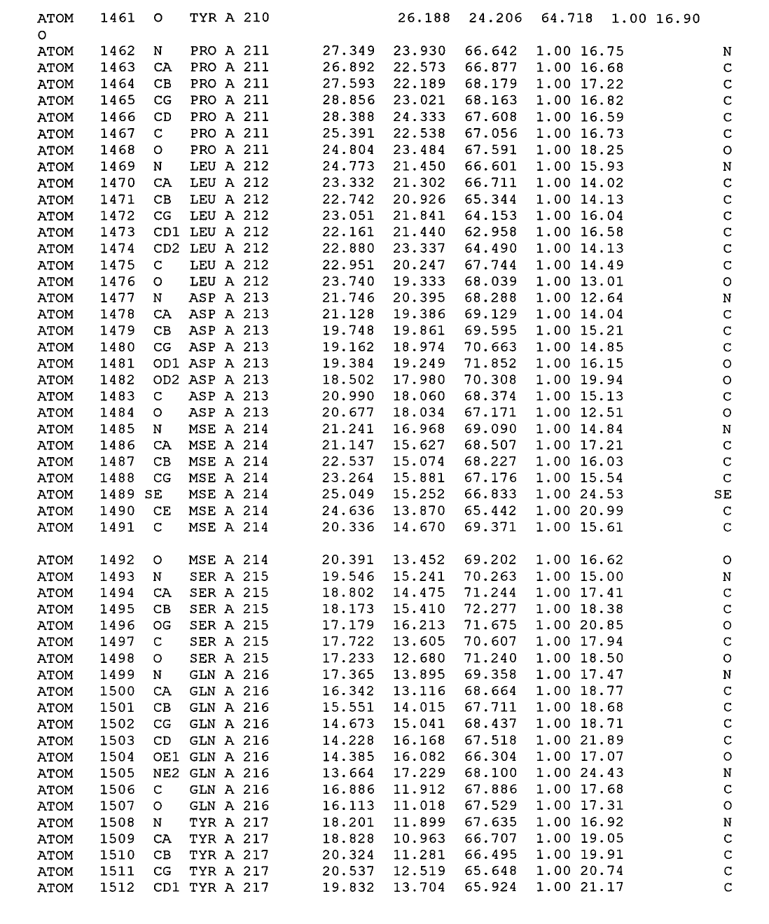

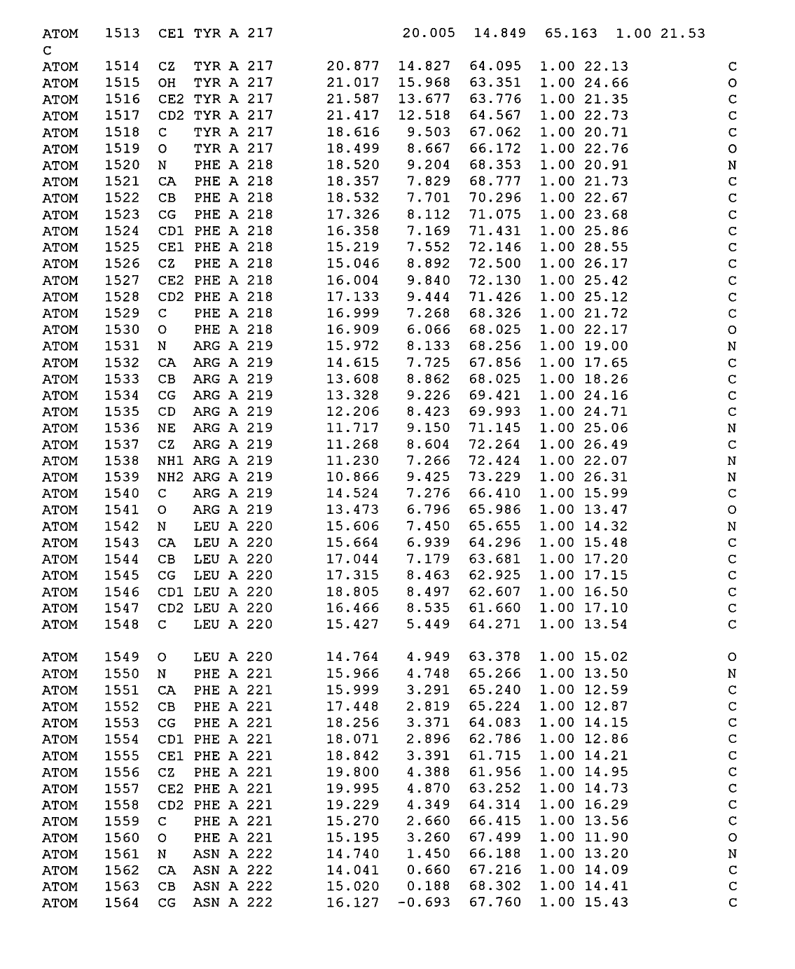

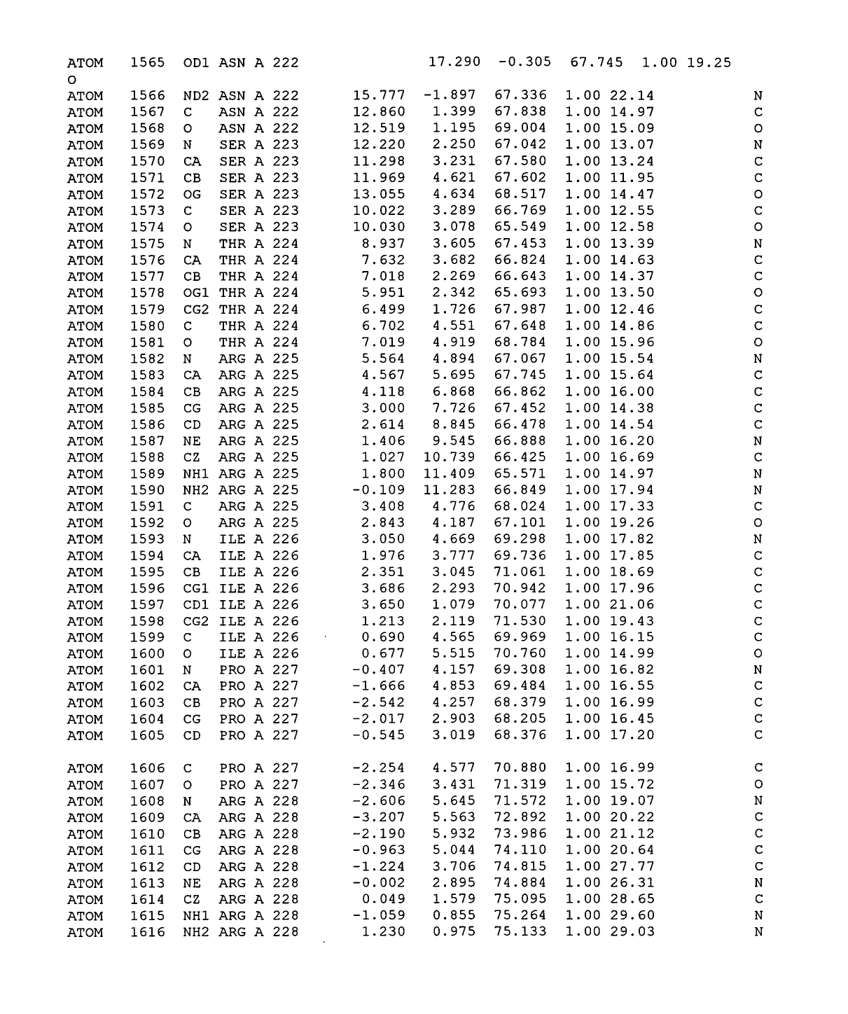

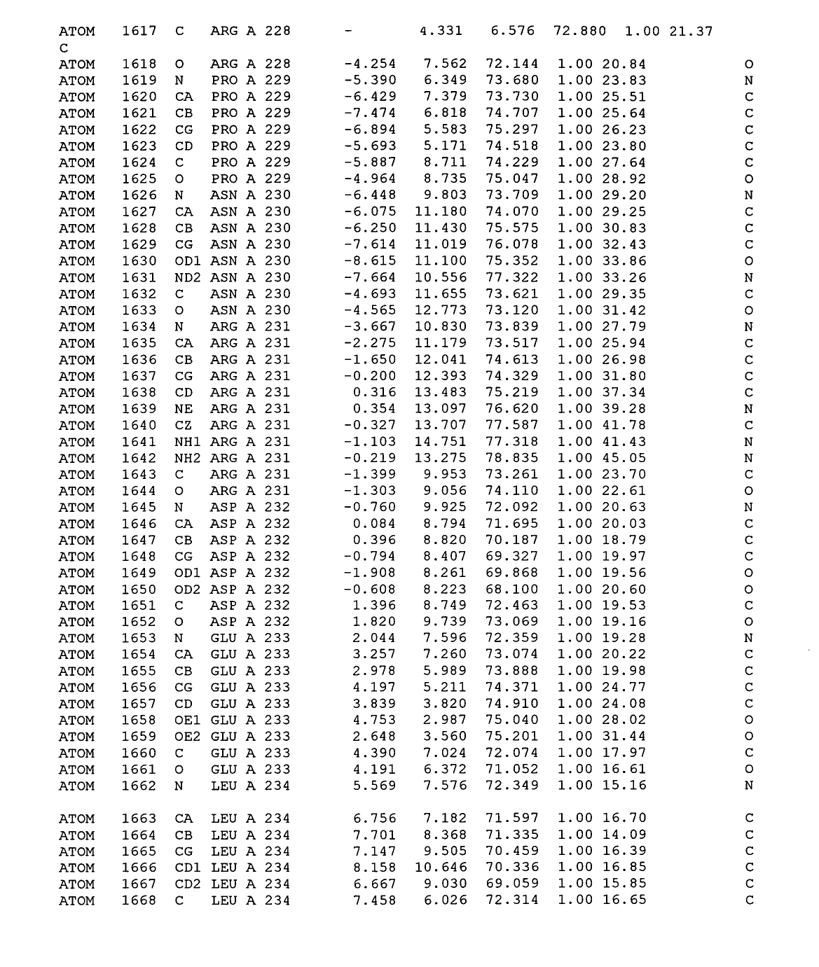

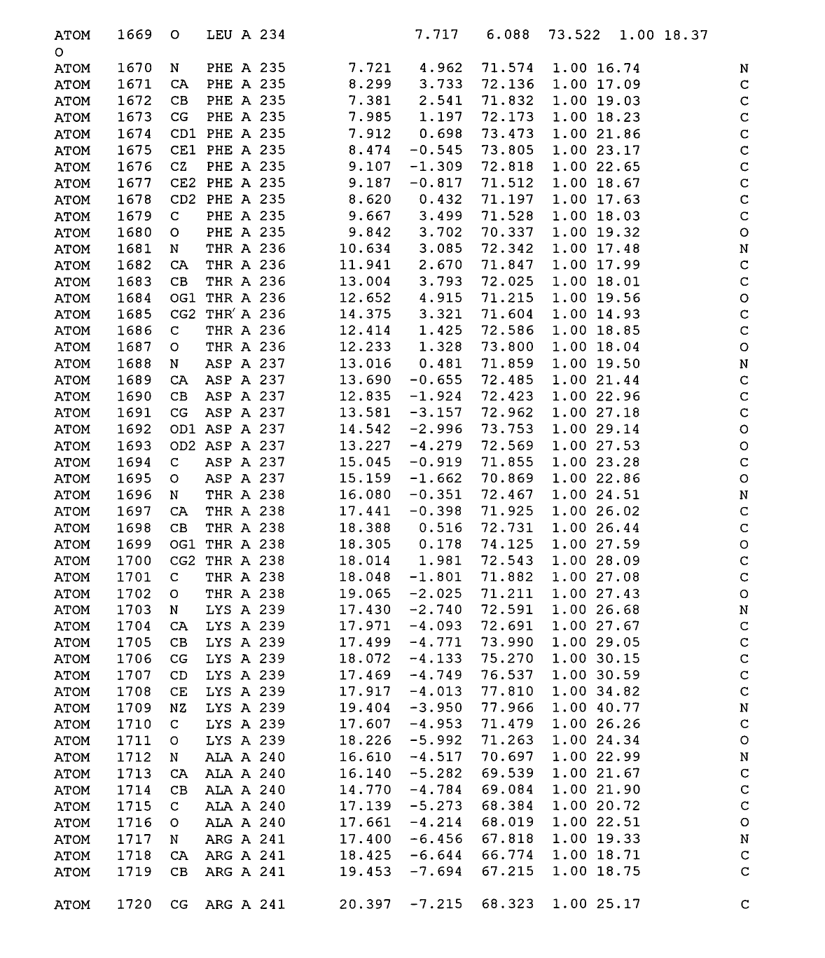

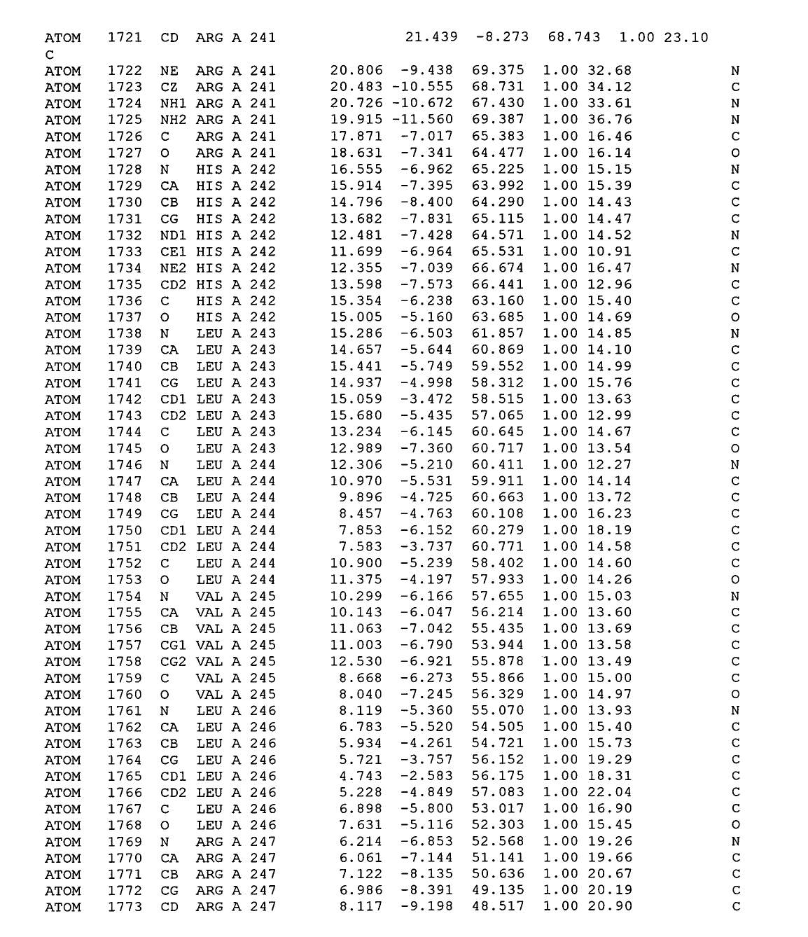

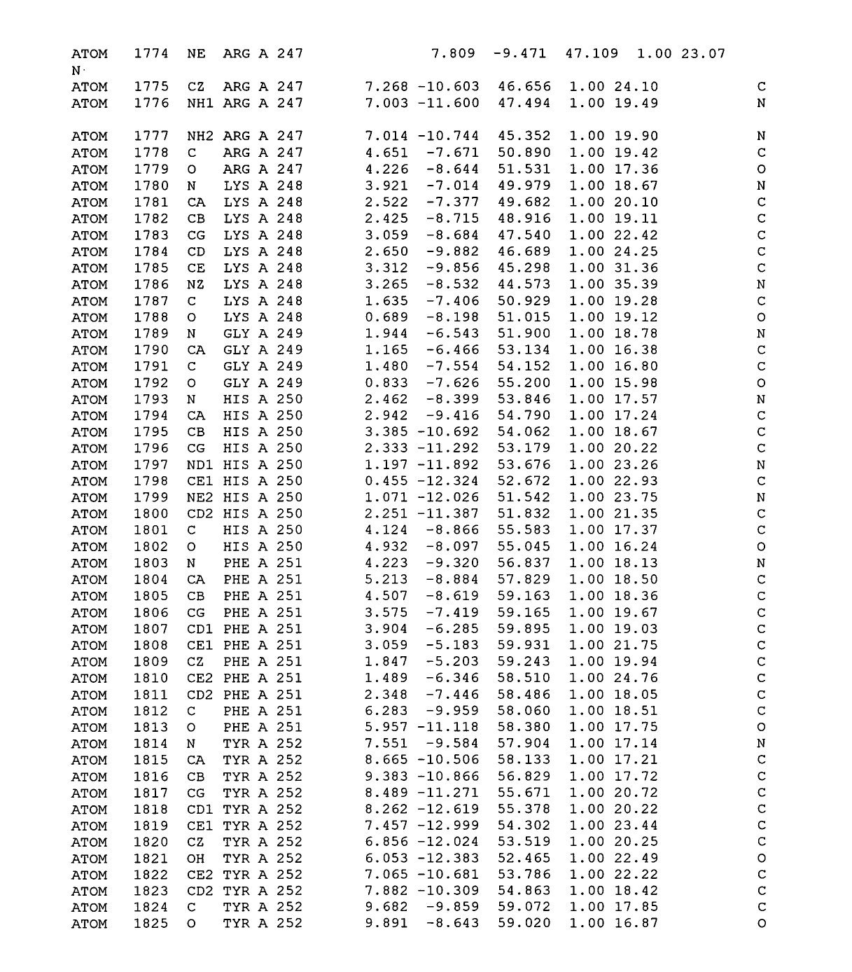

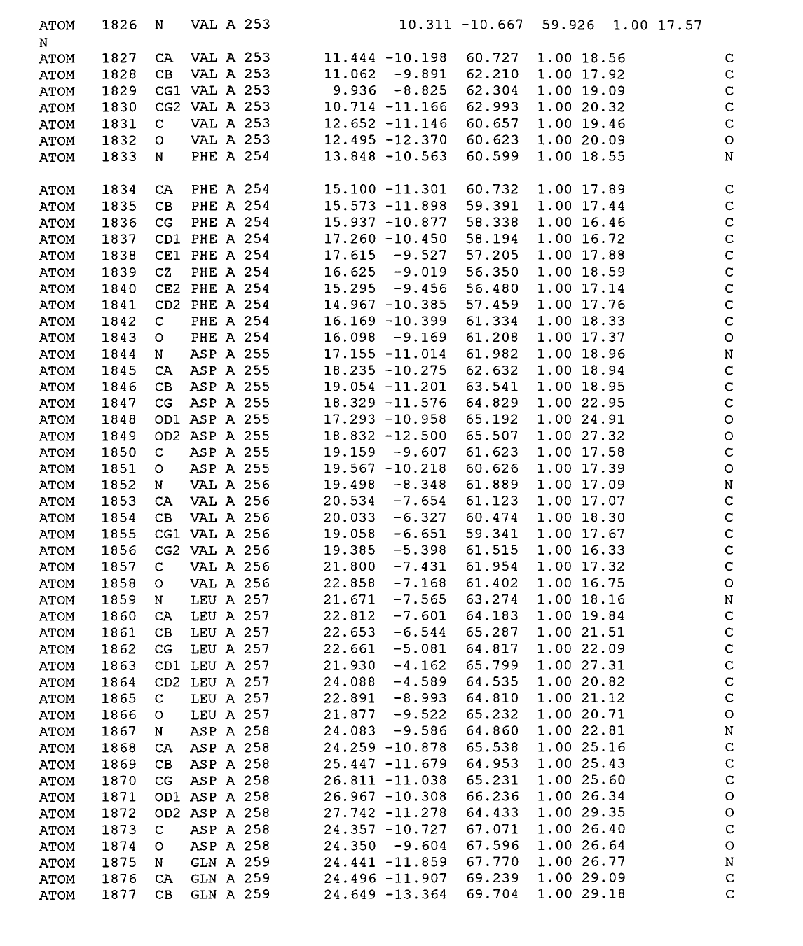

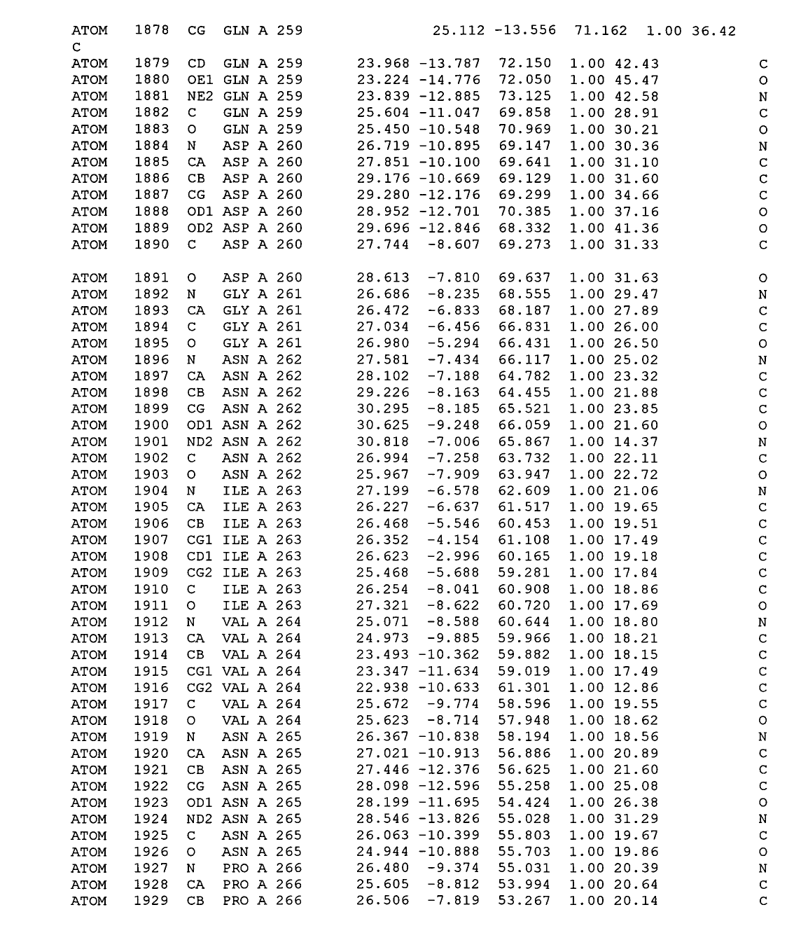

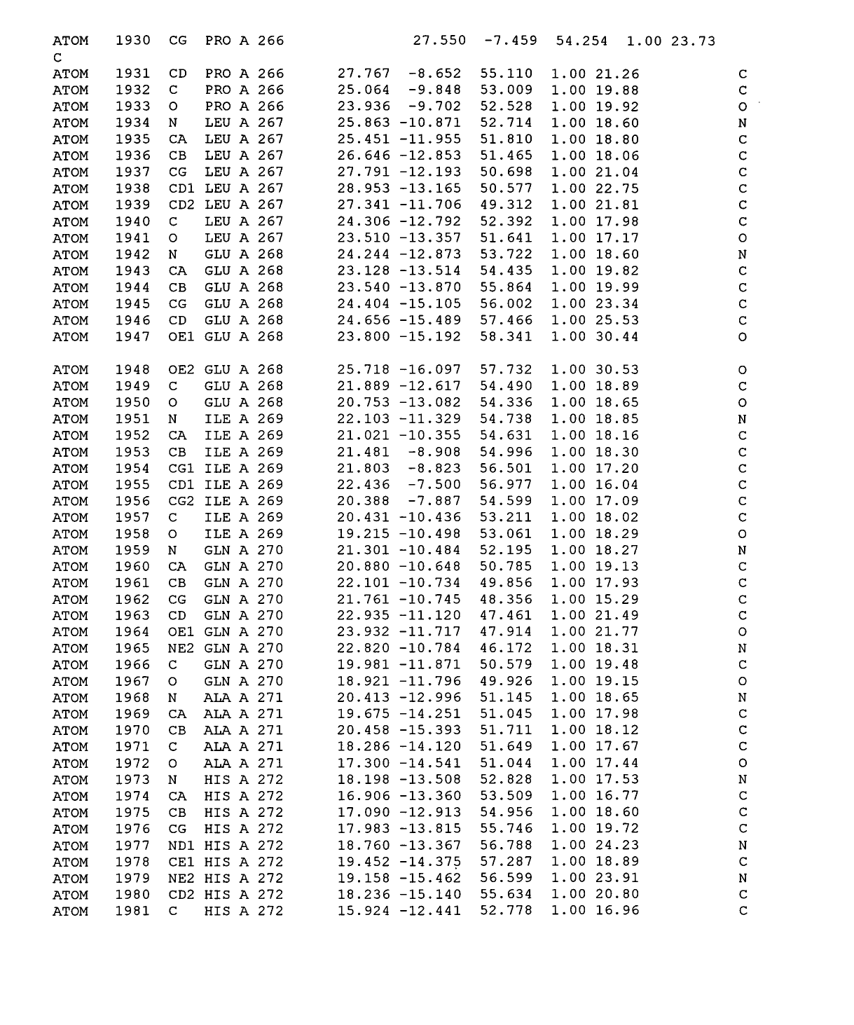

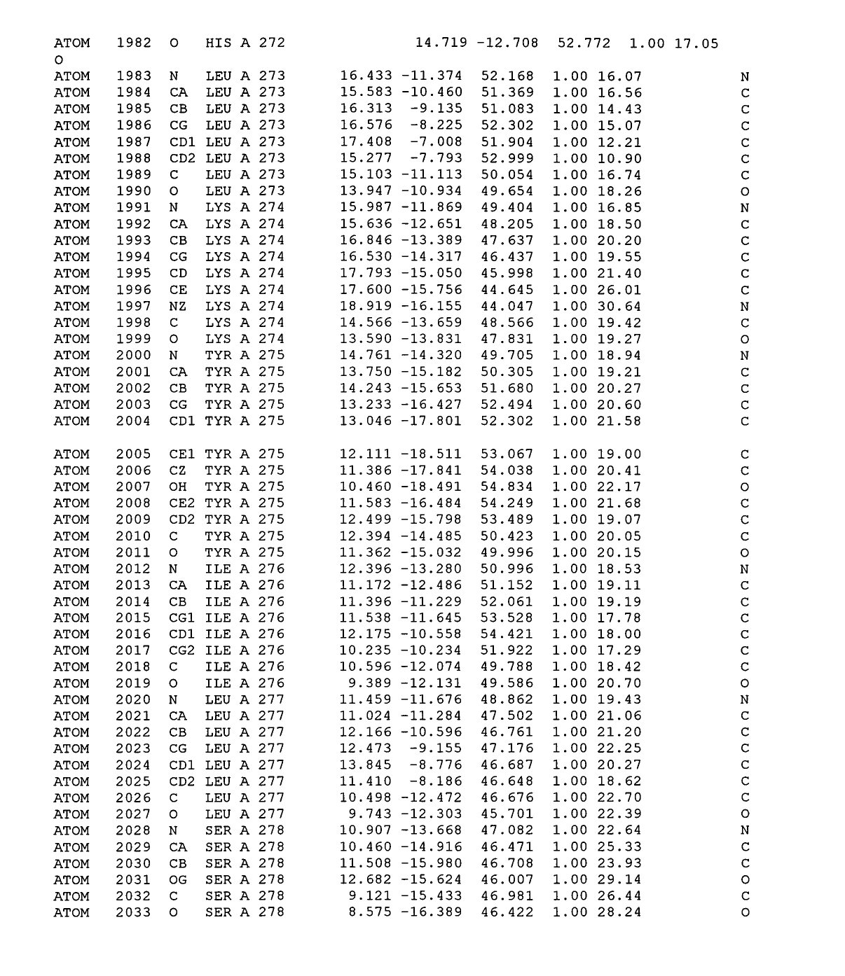

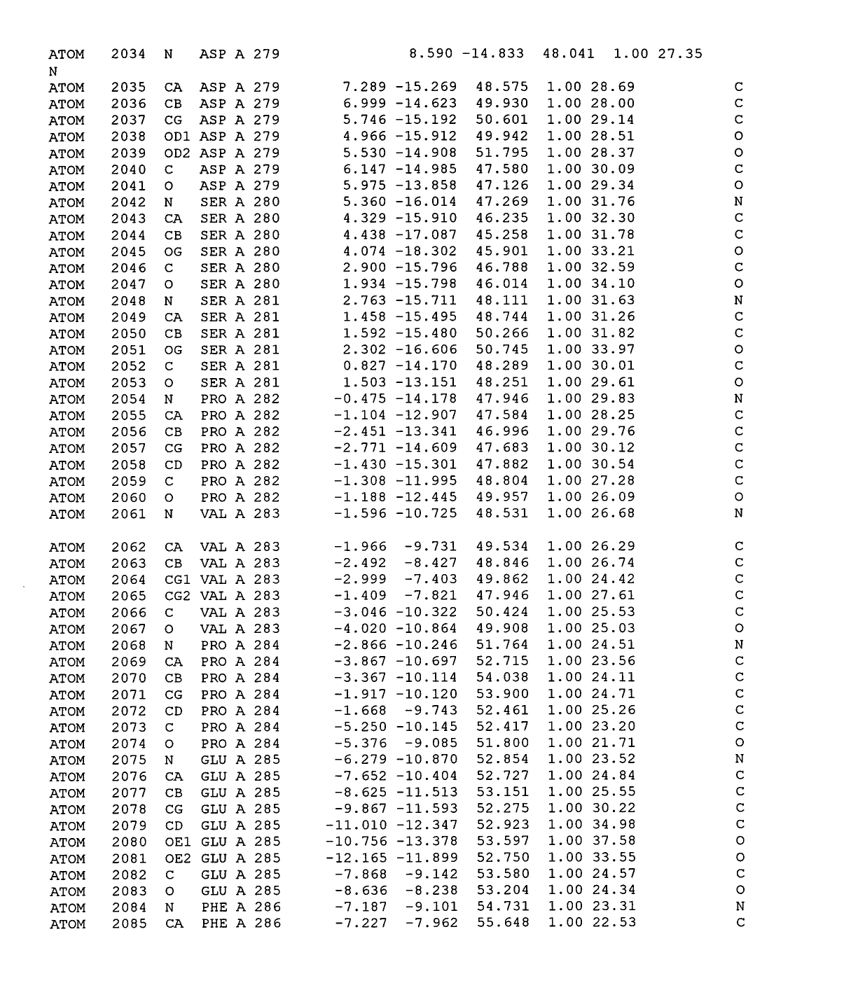

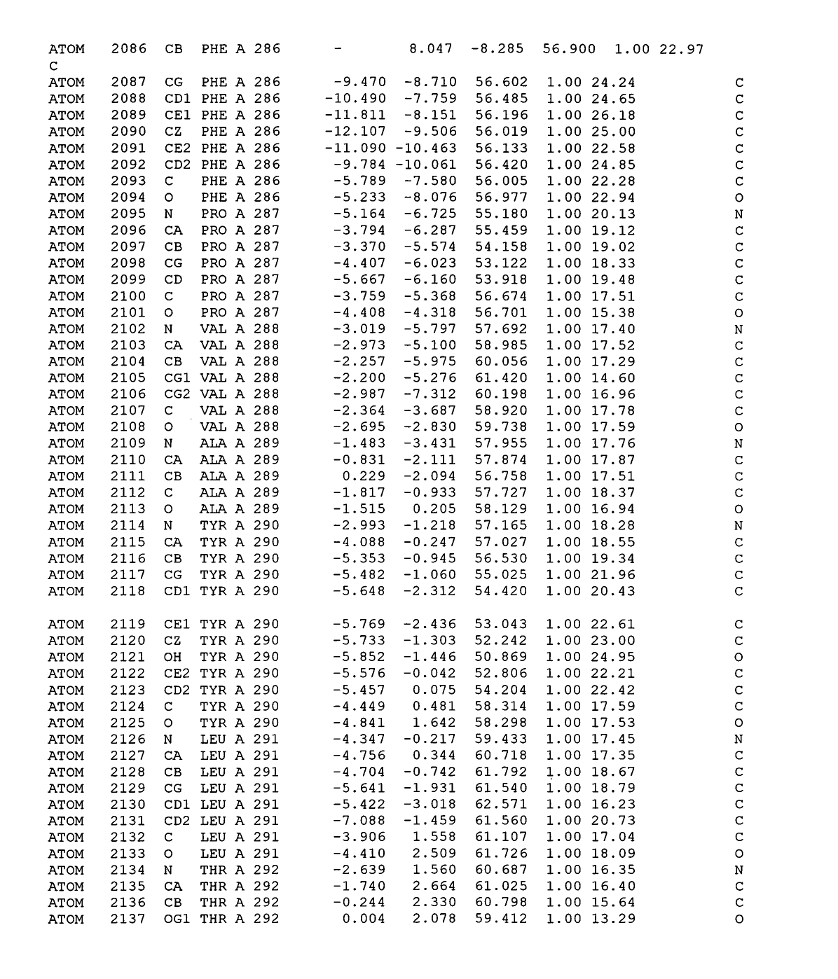

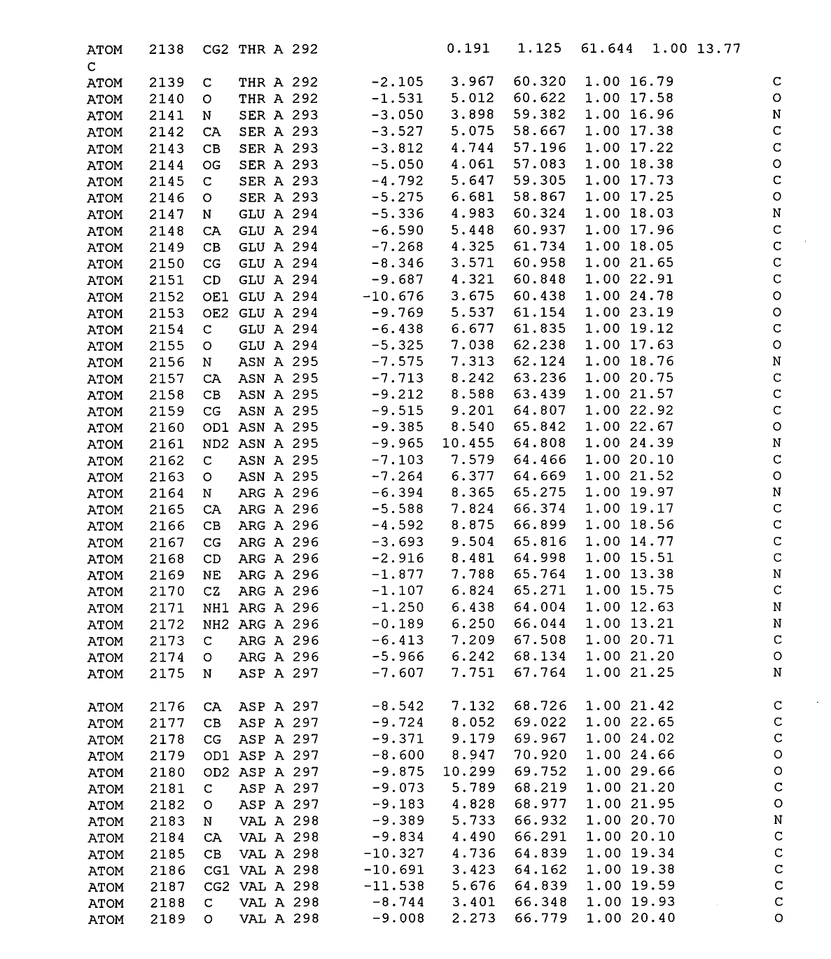

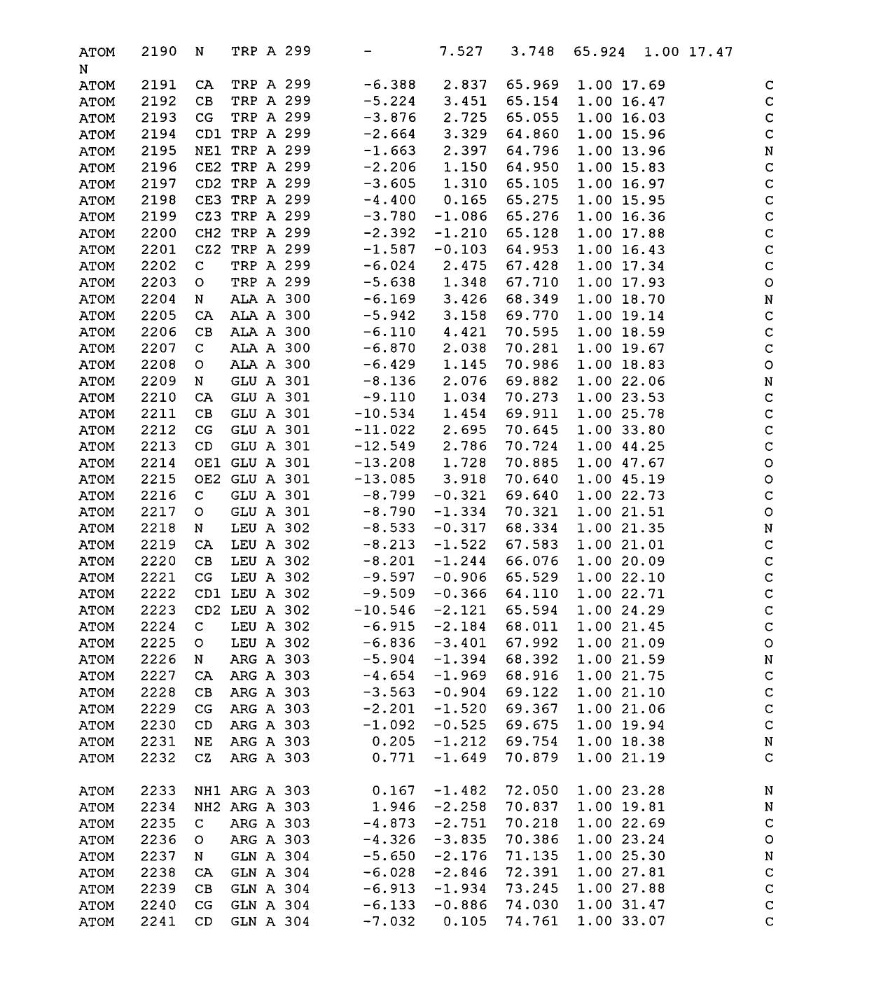

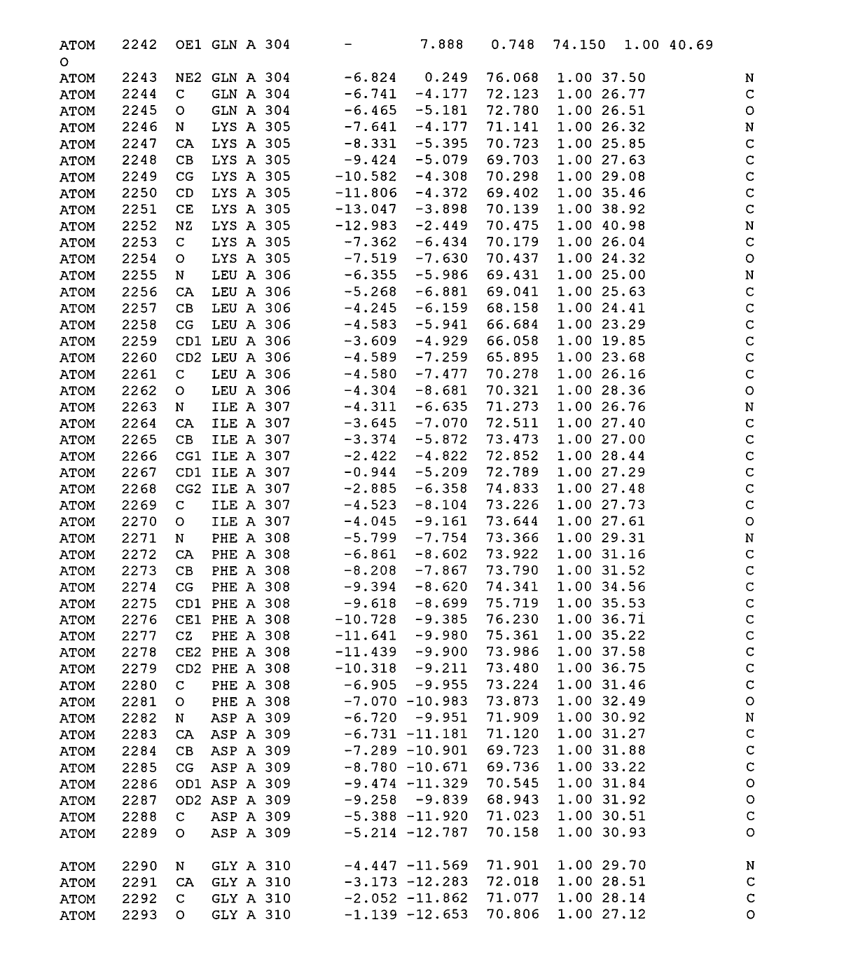

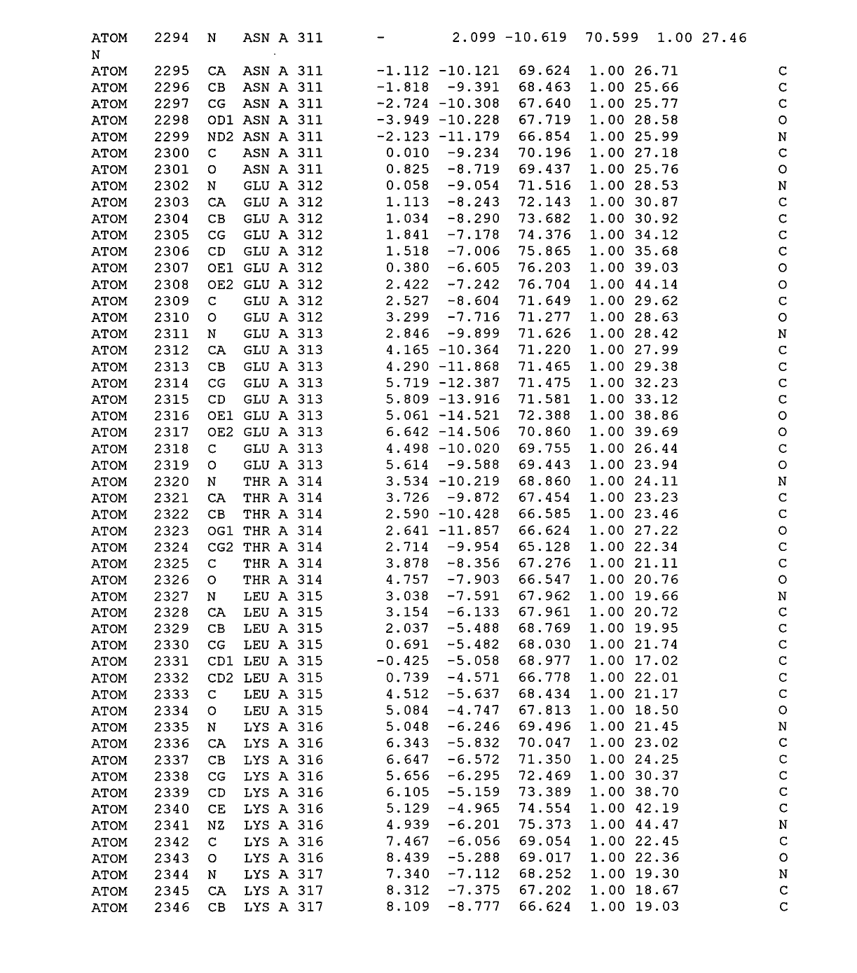

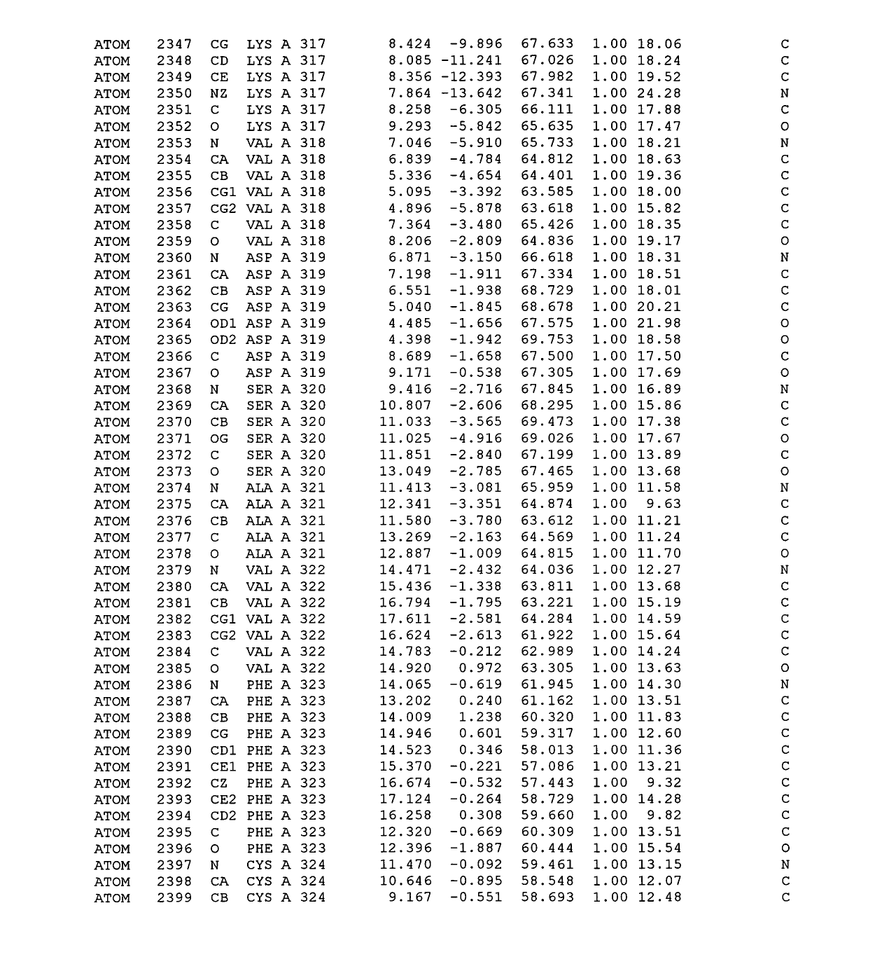

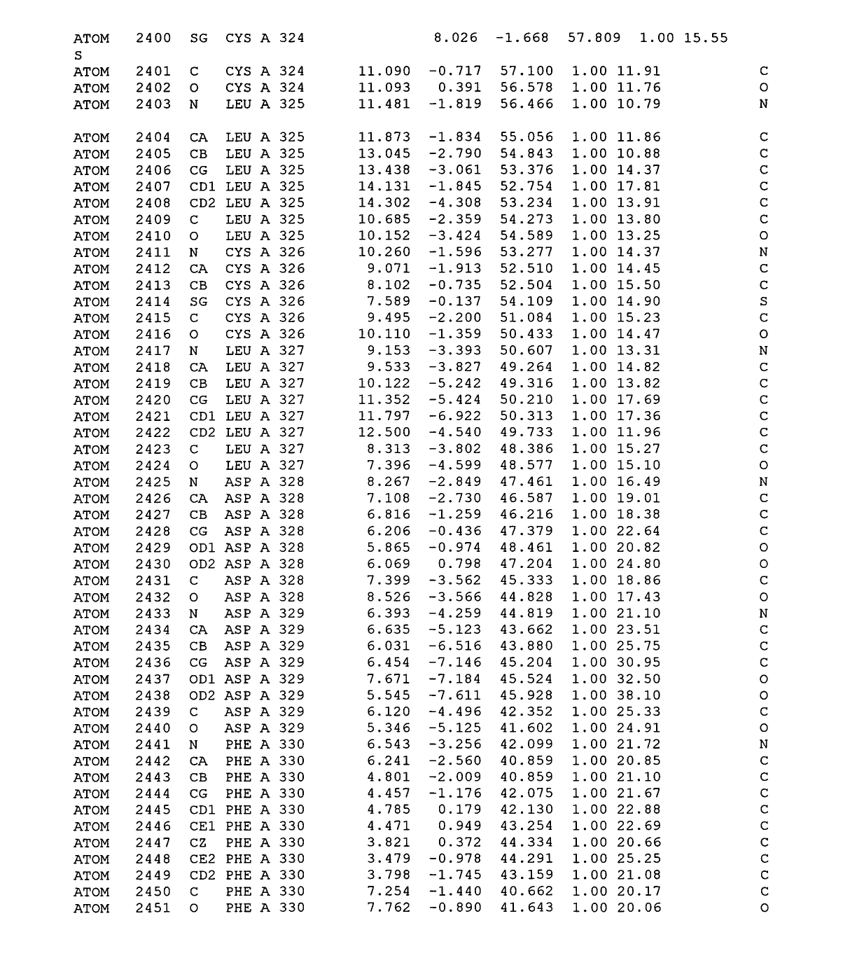

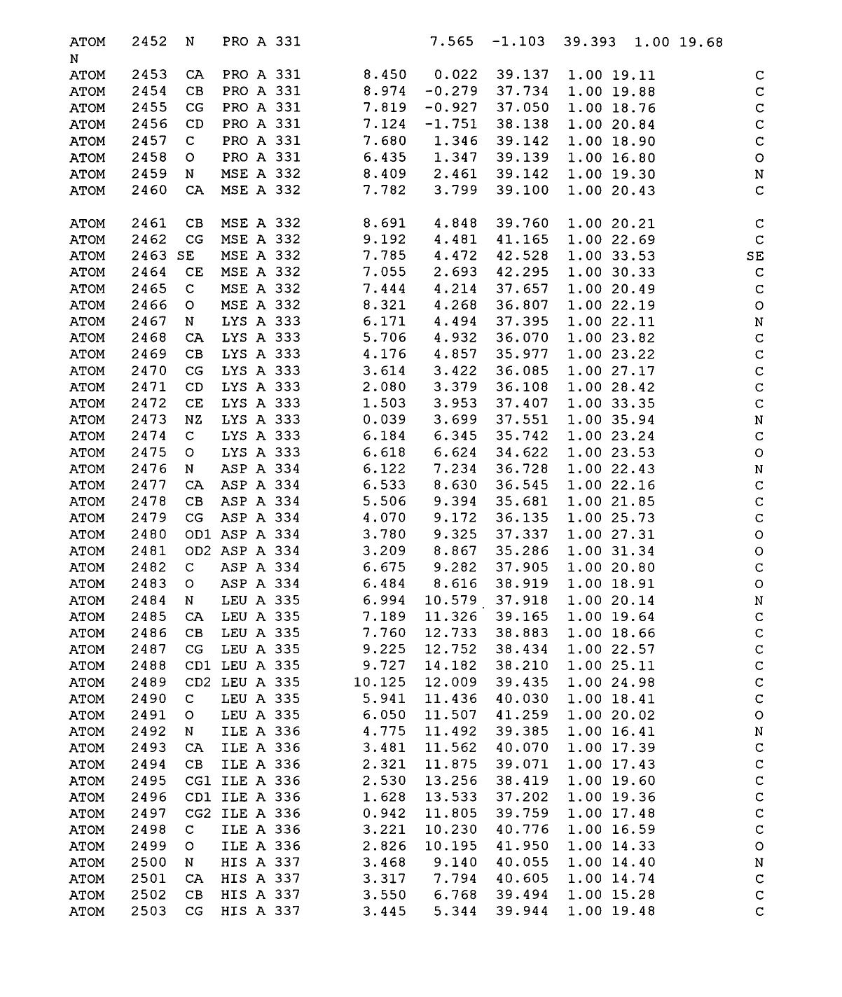

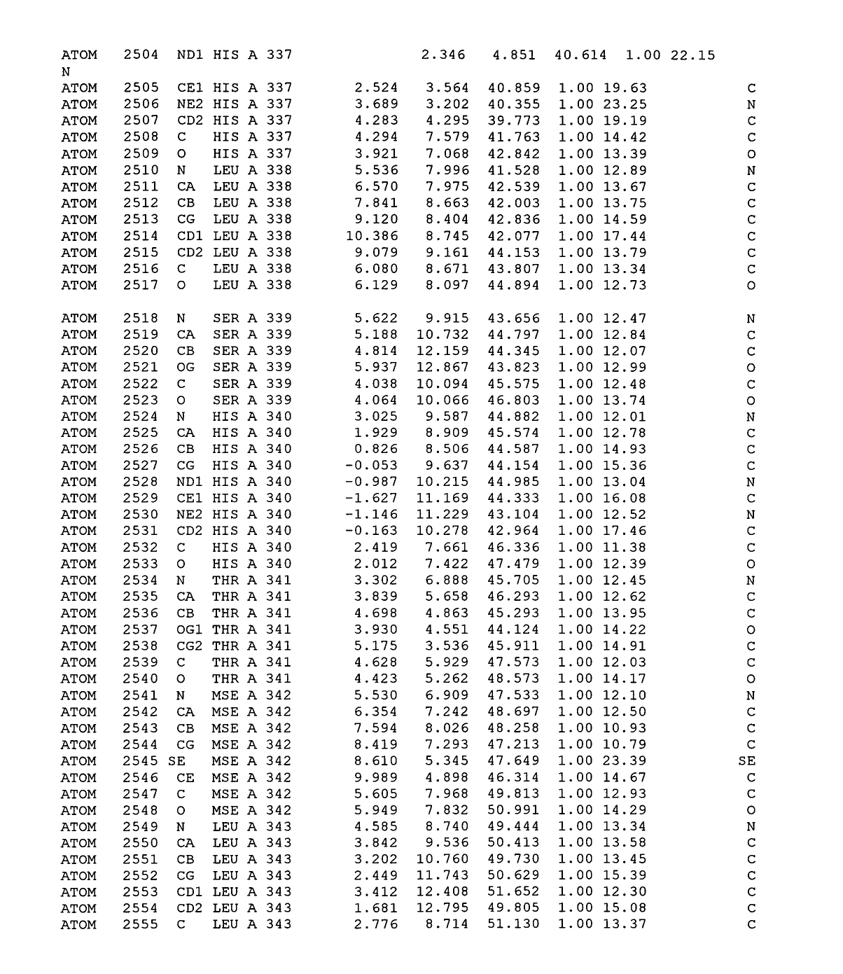

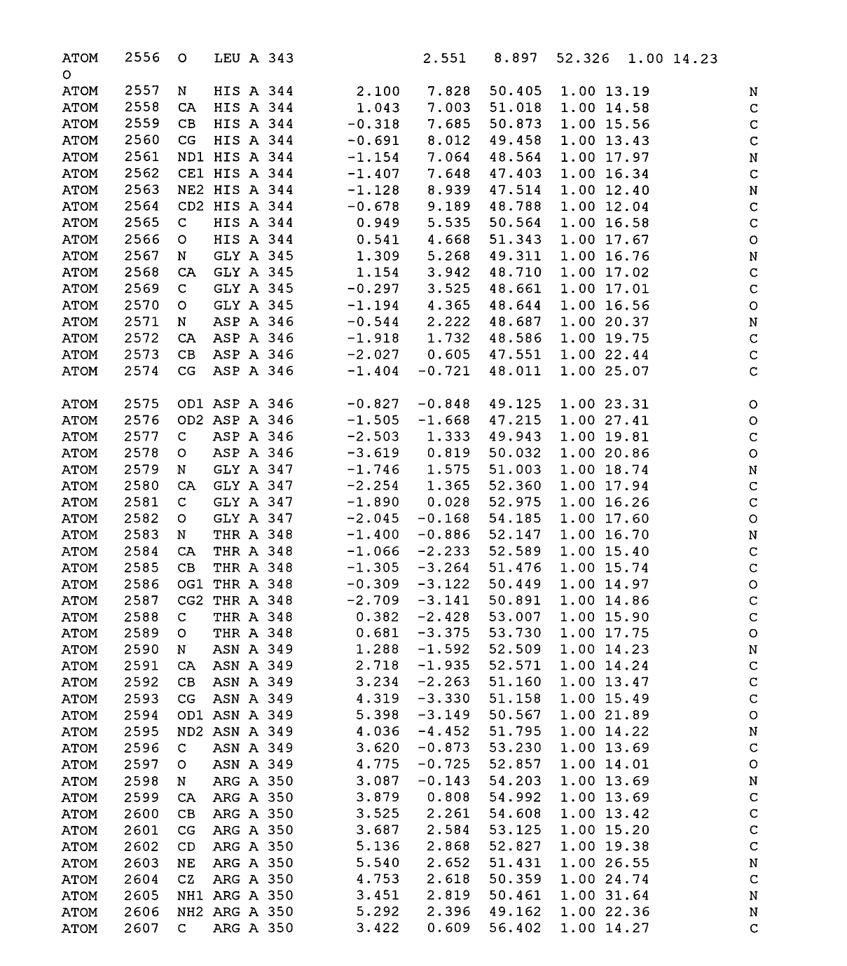

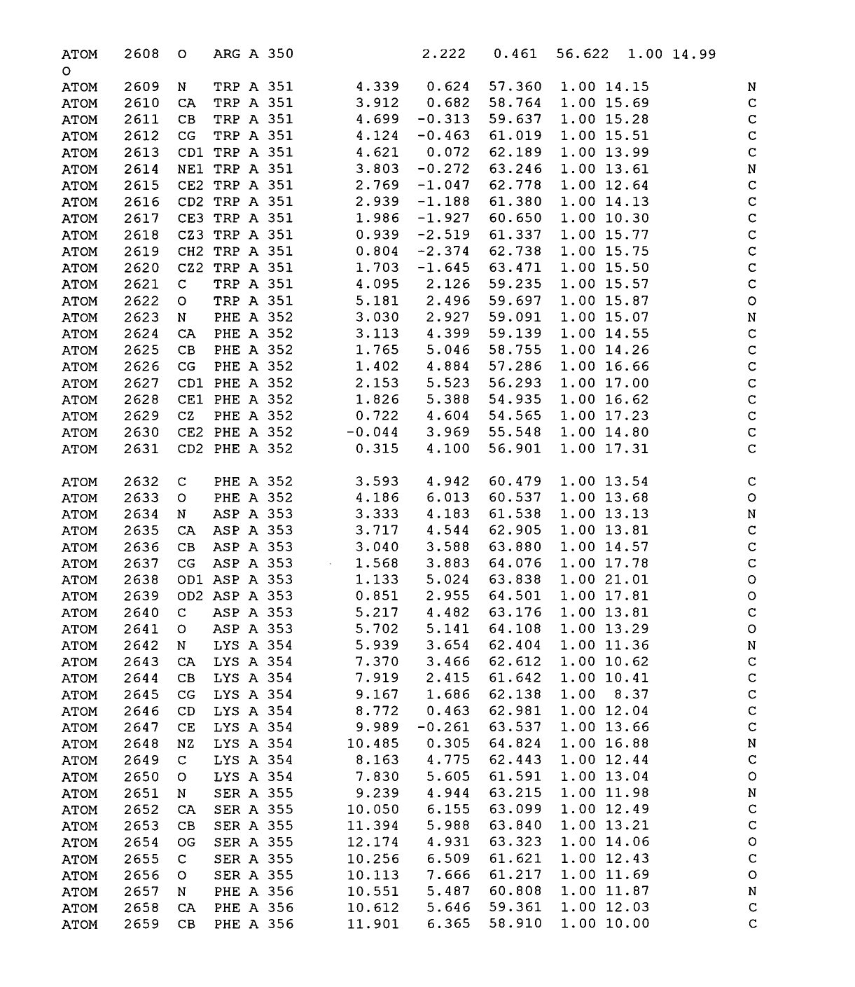

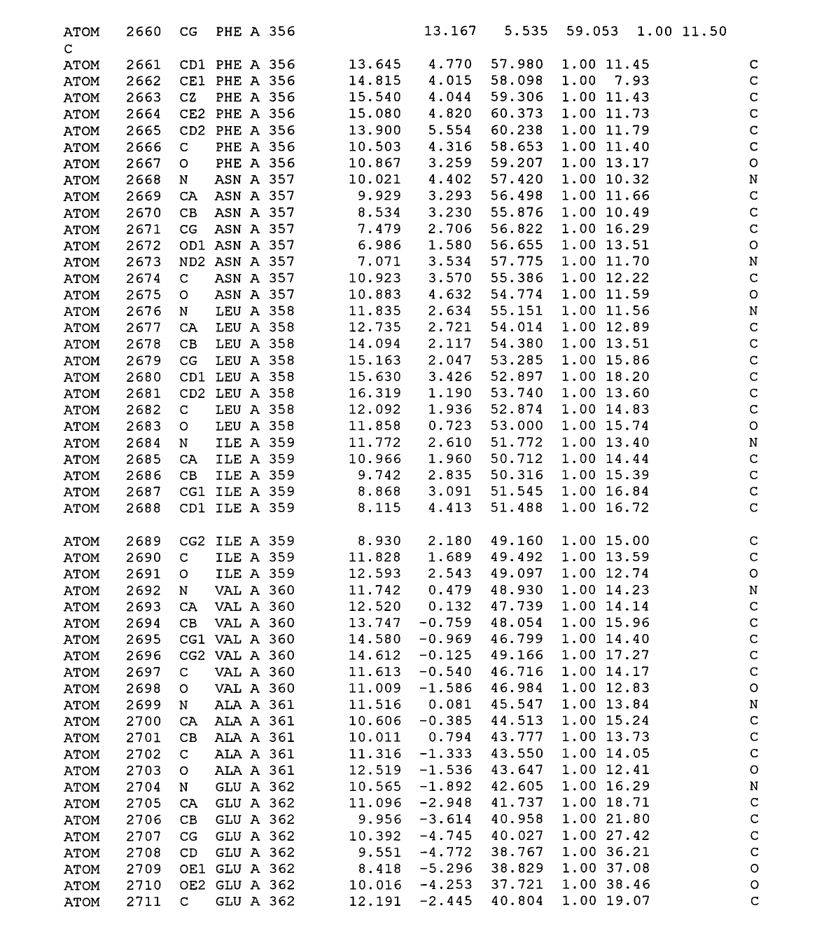

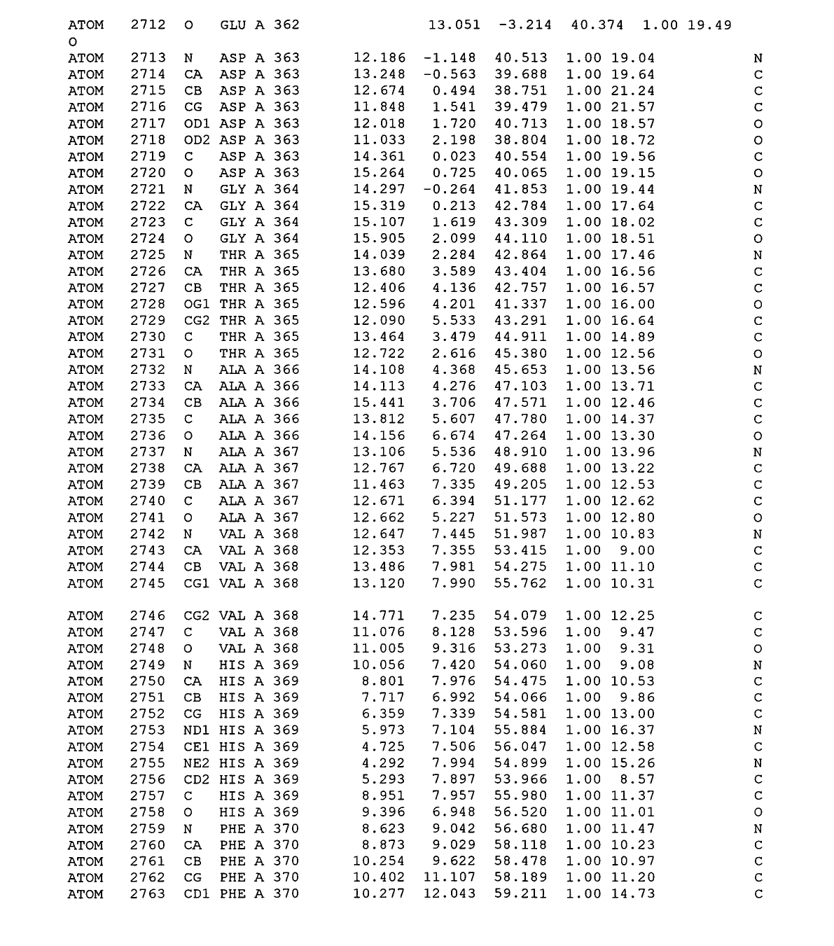

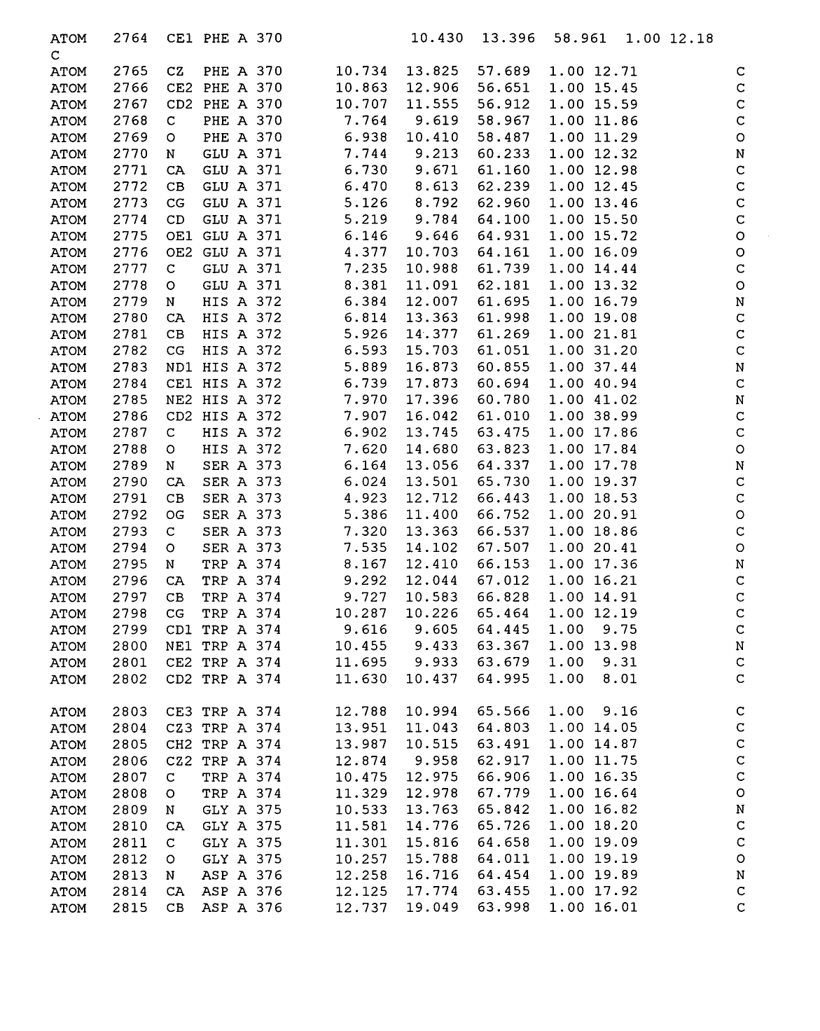

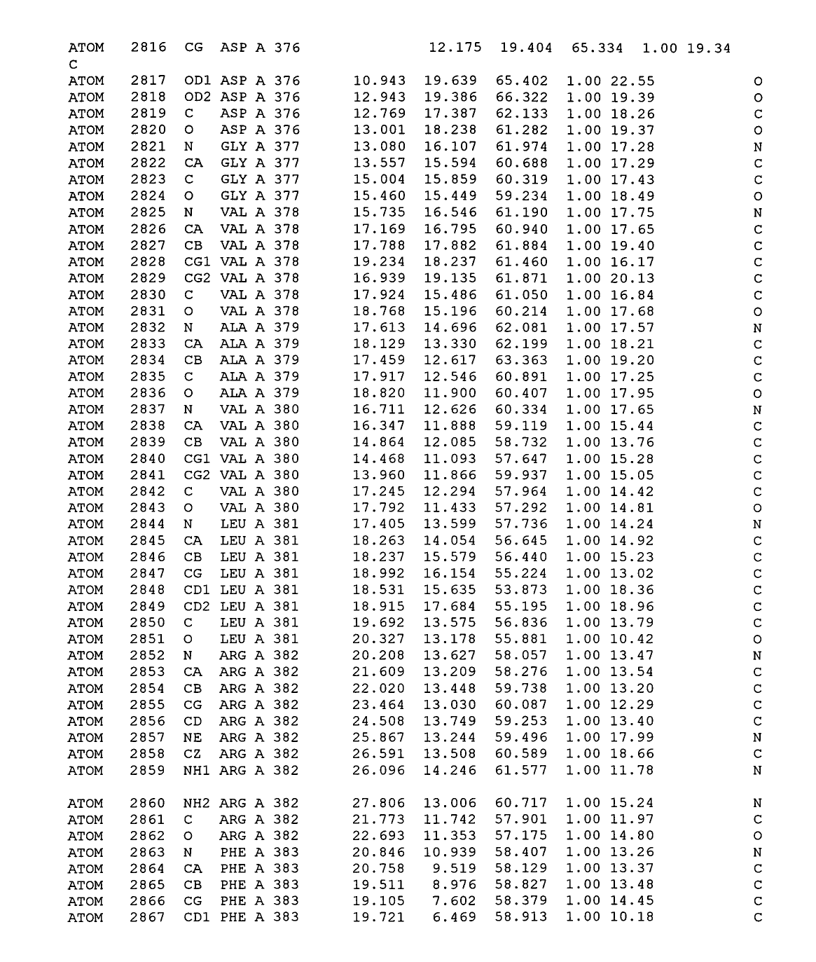

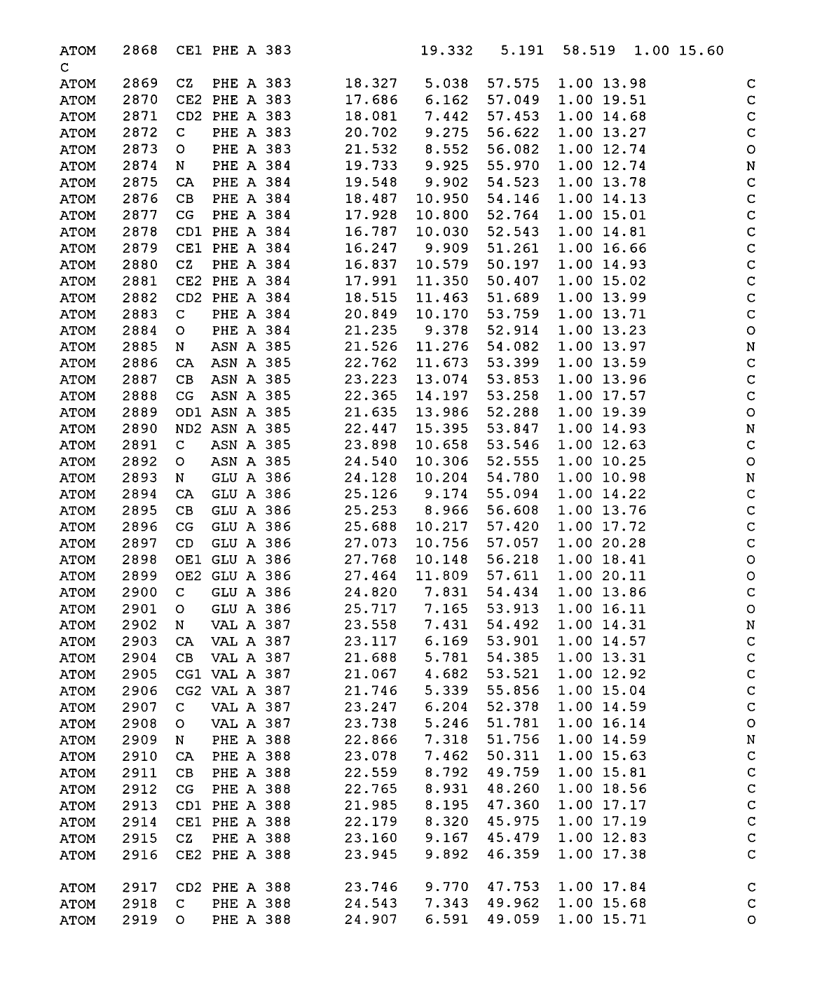

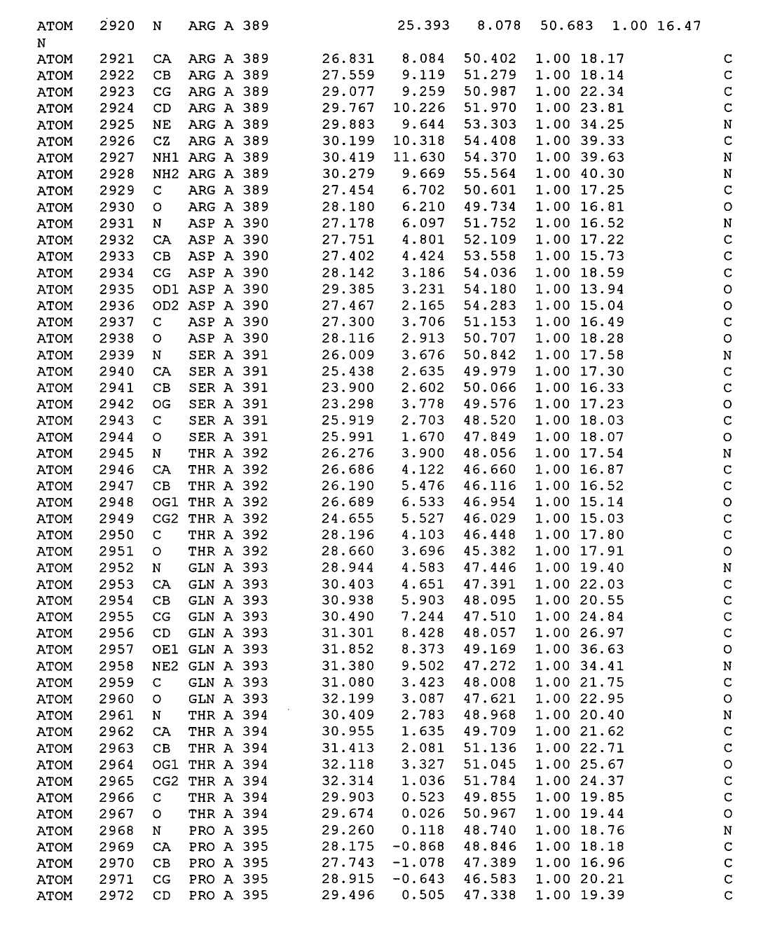

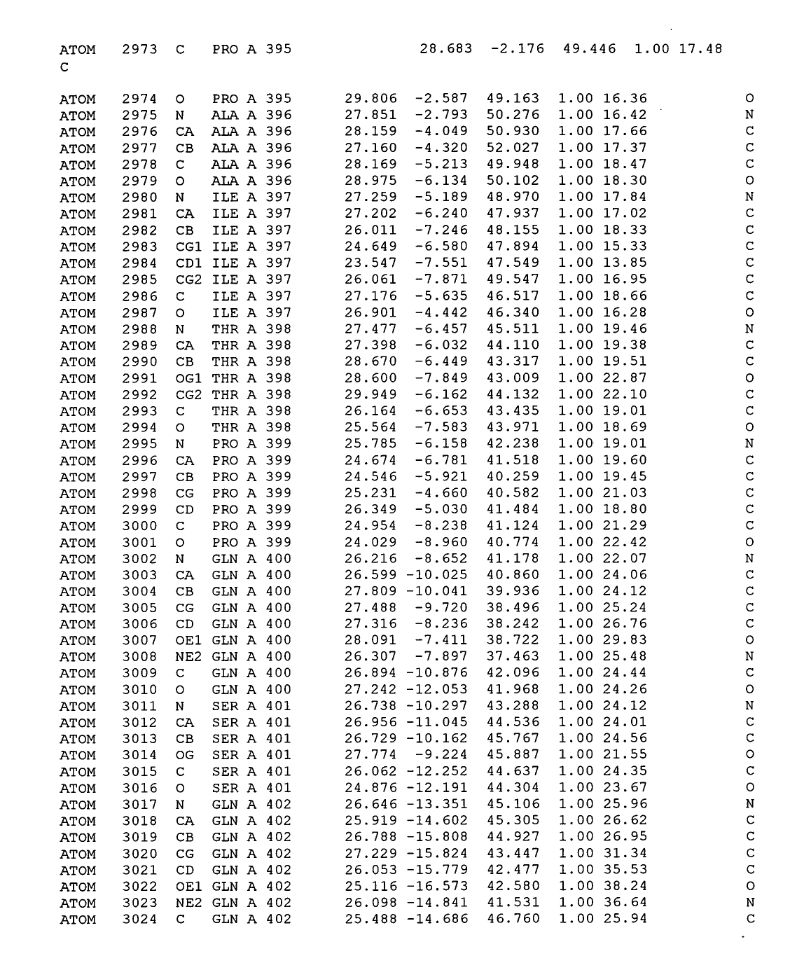

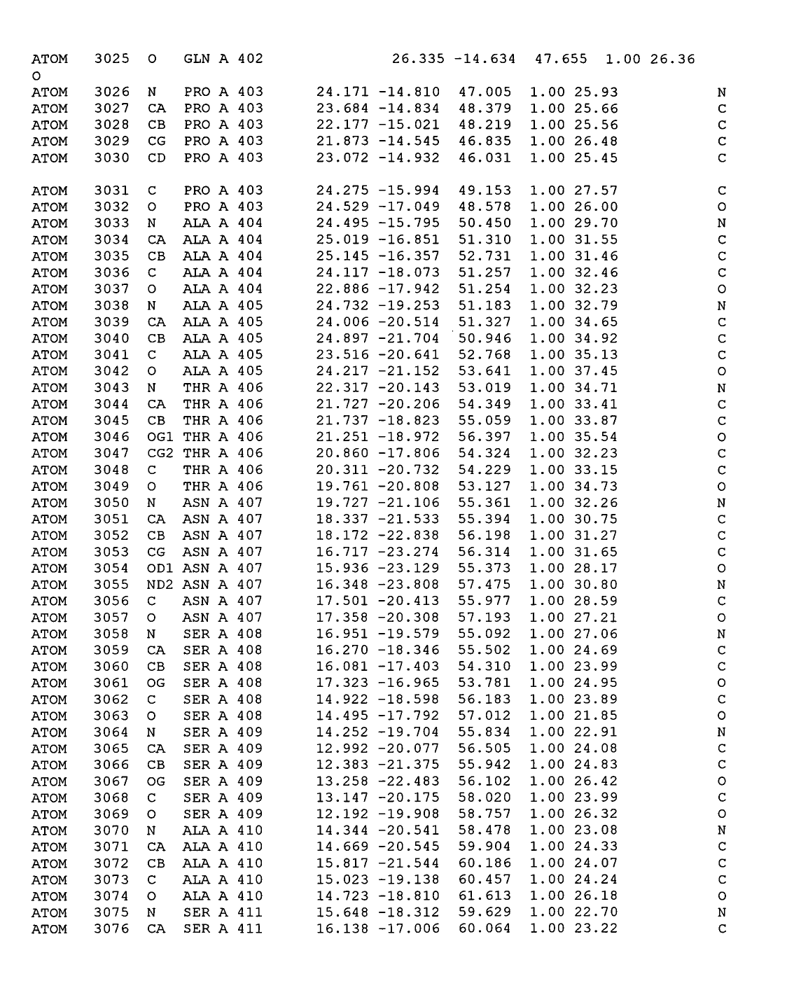

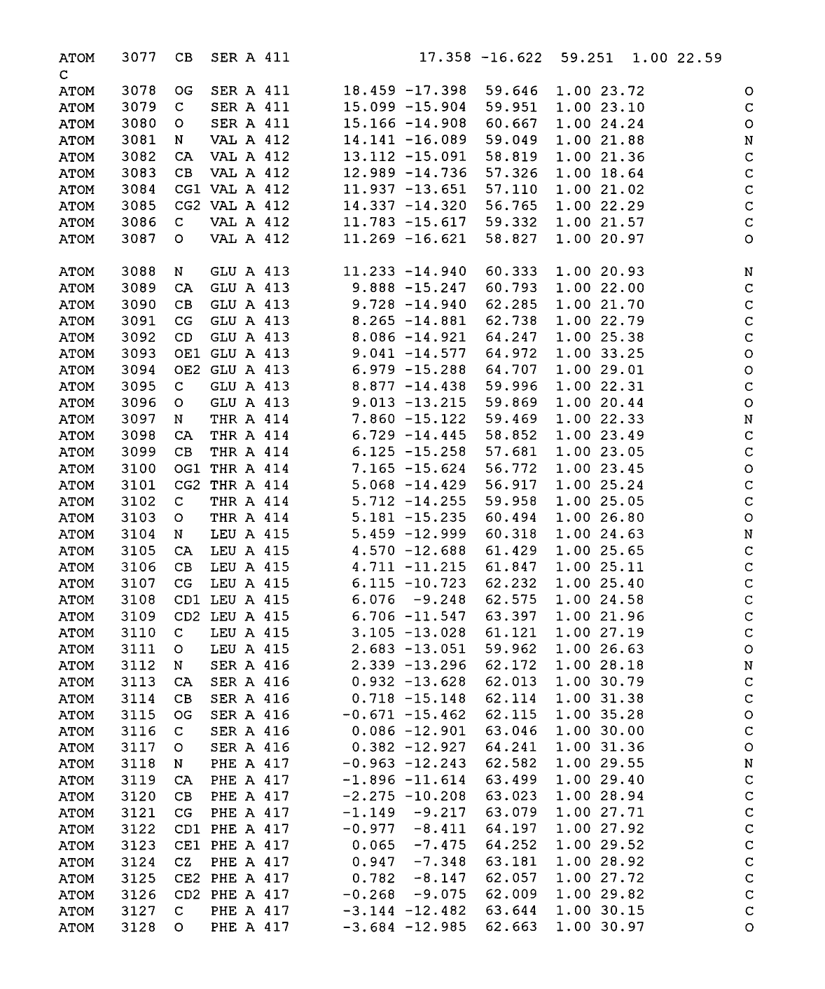

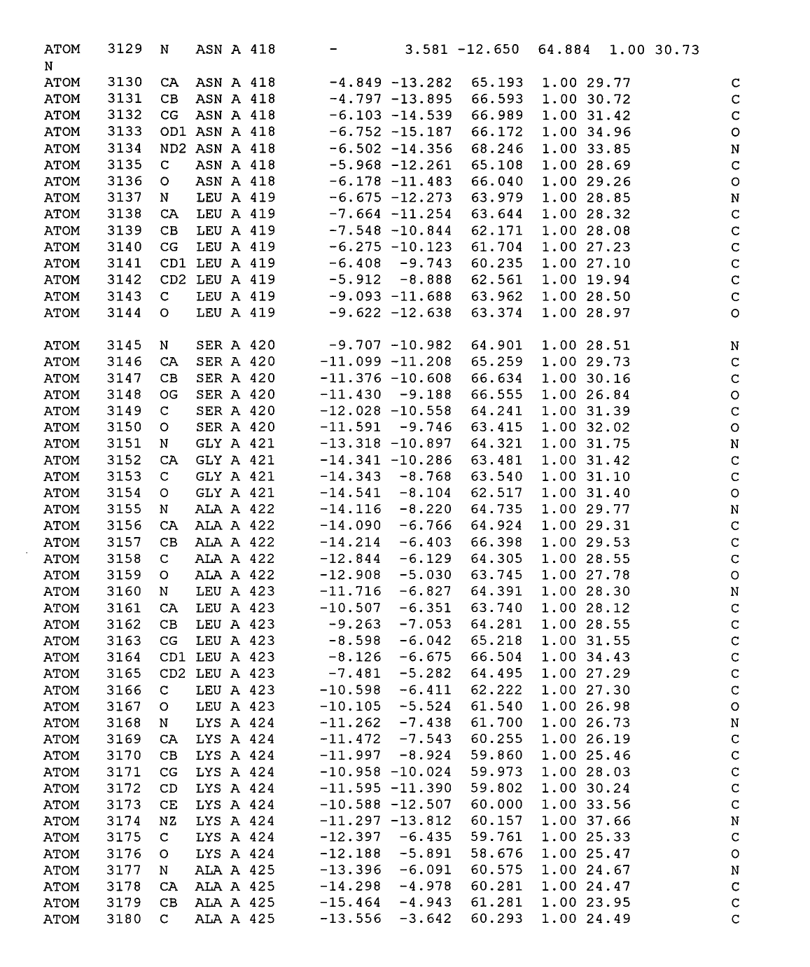

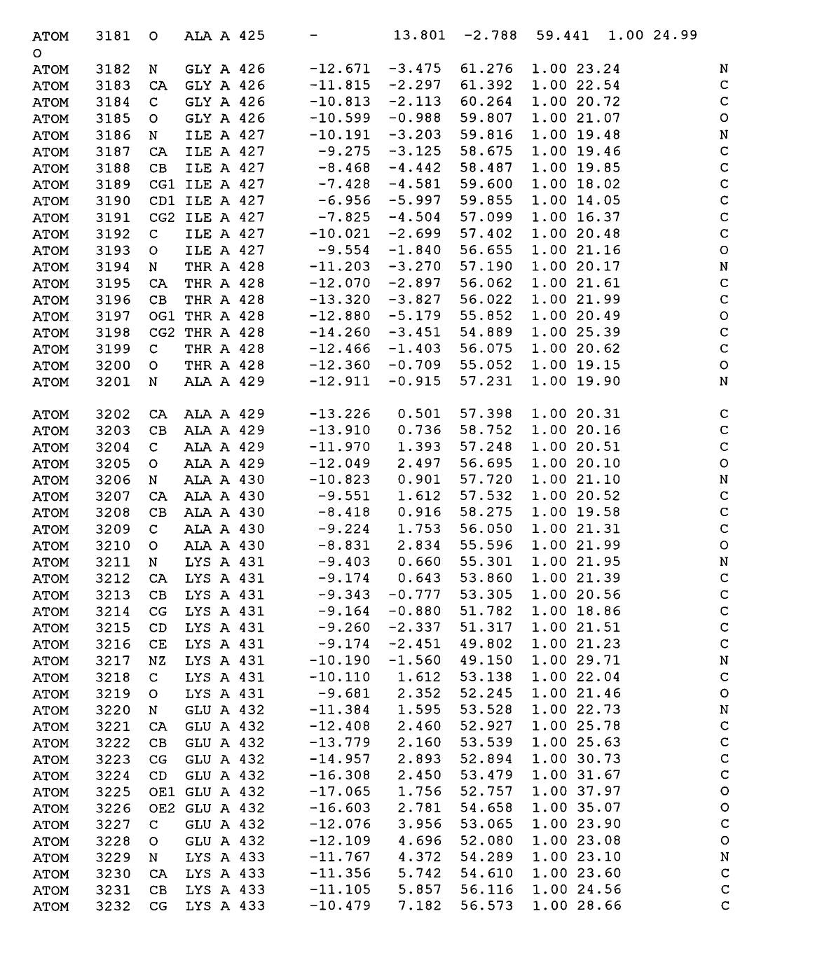

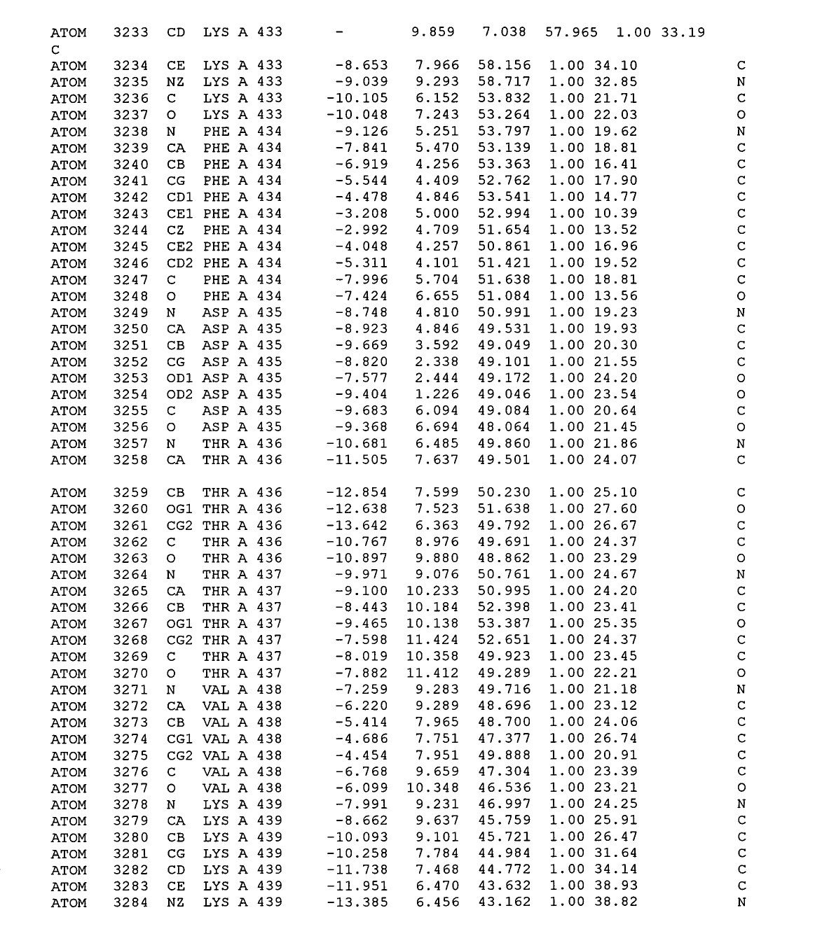

1. An isolated protein having the structure defined by the structural coordinates as set forth in FIG. 4.

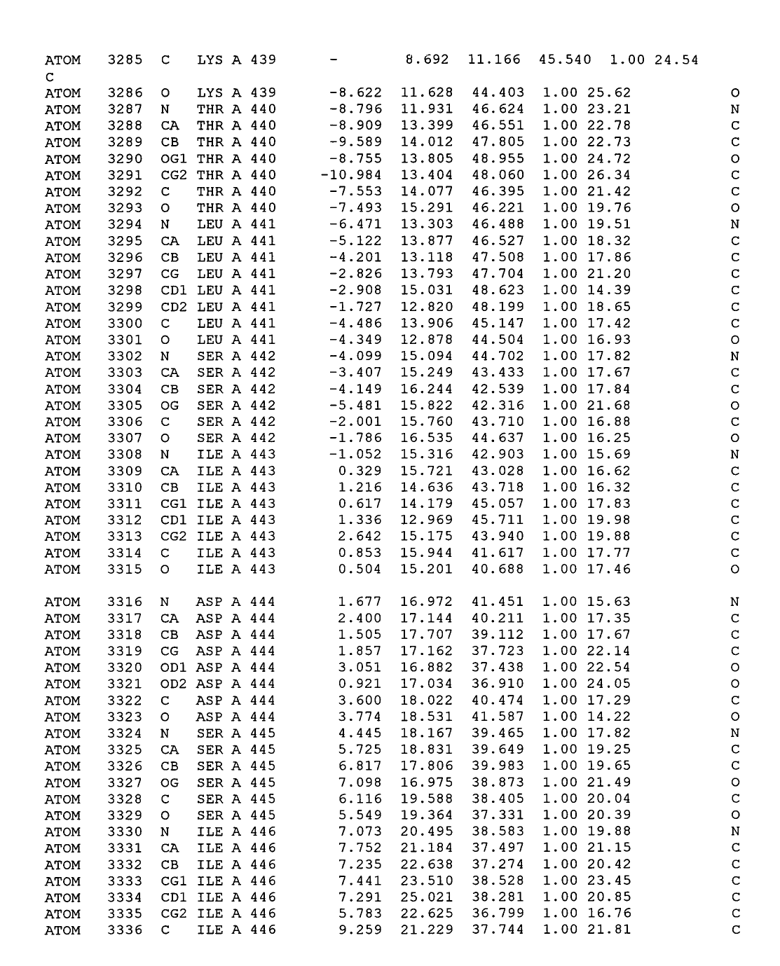

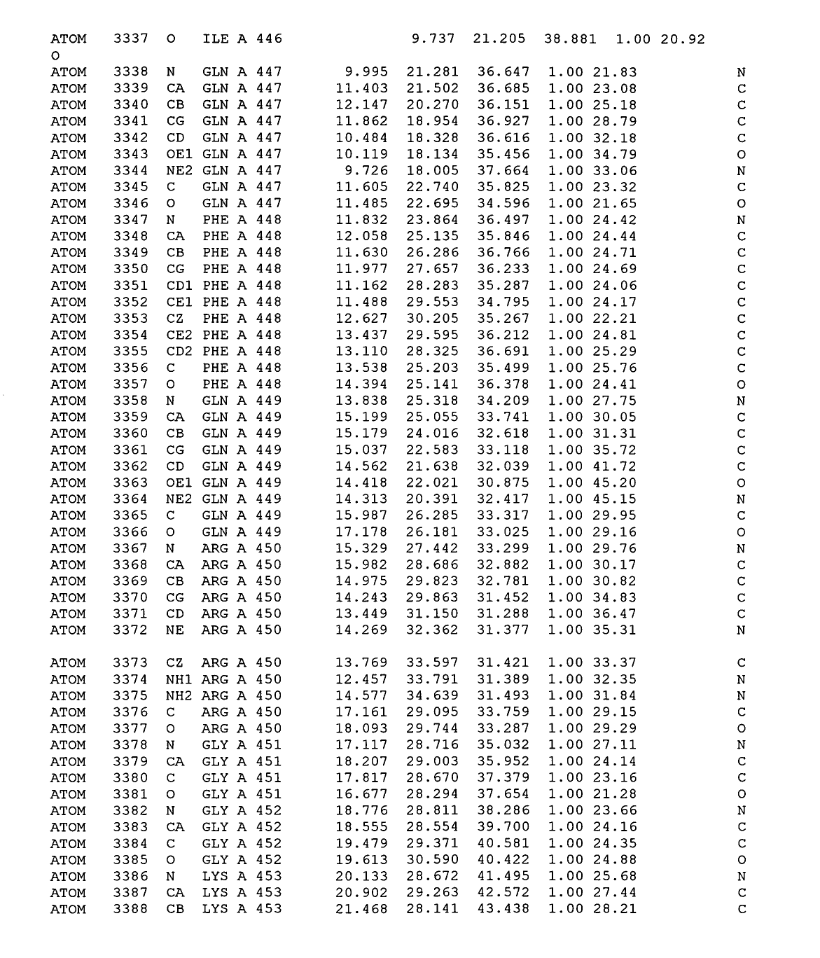

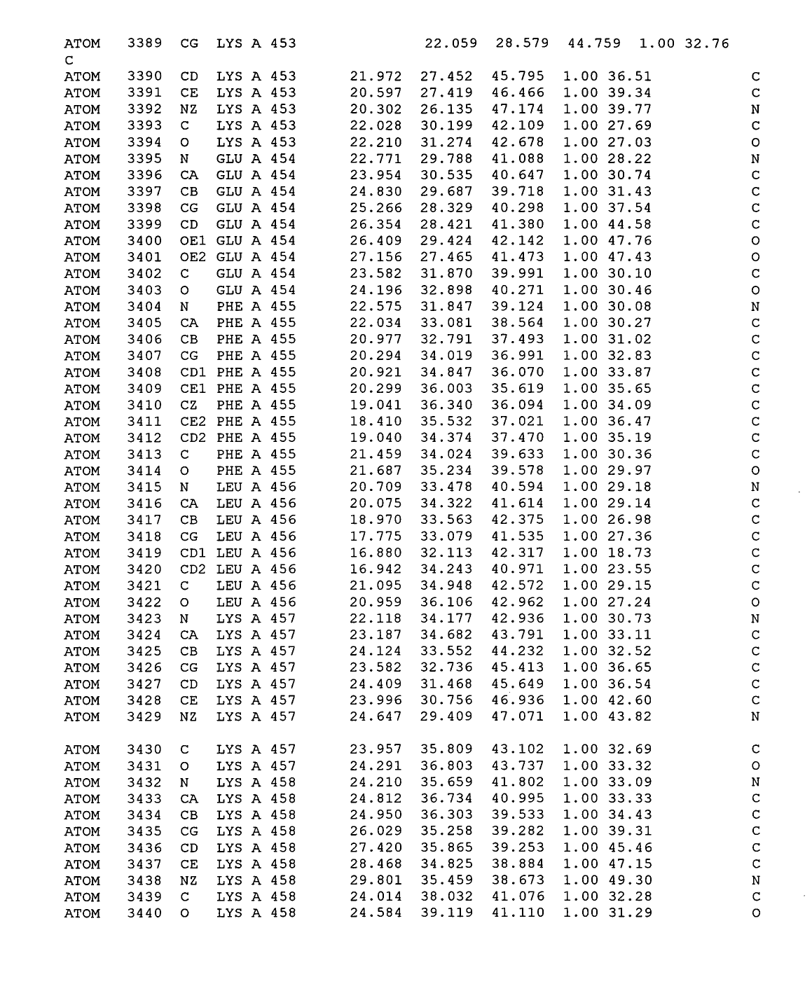

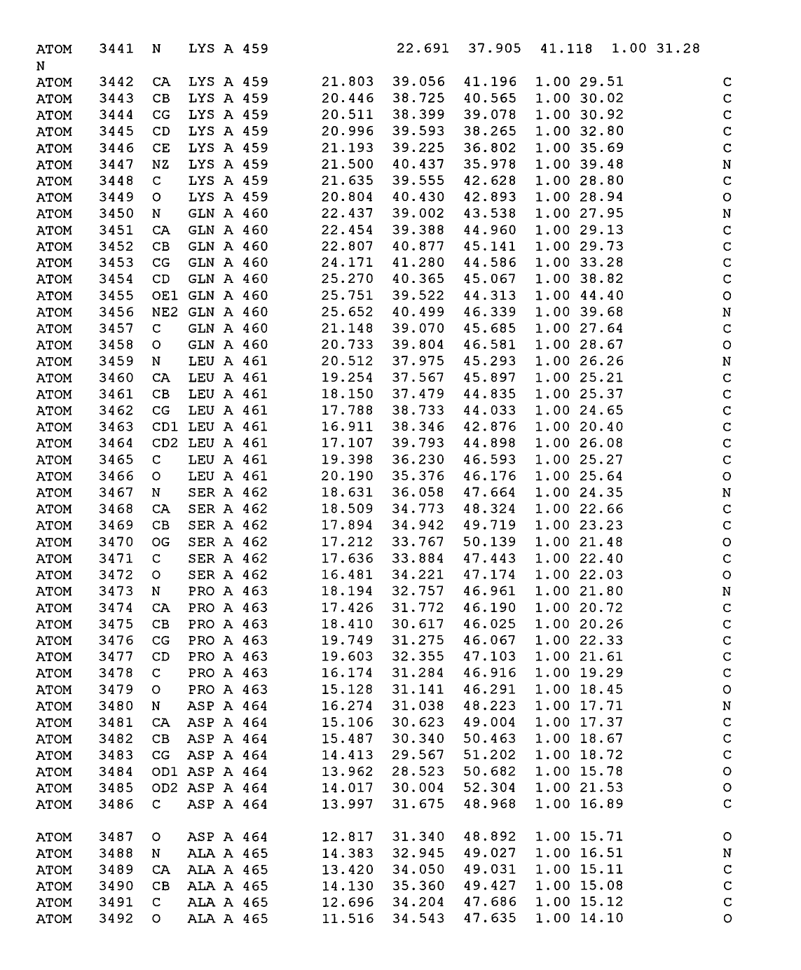

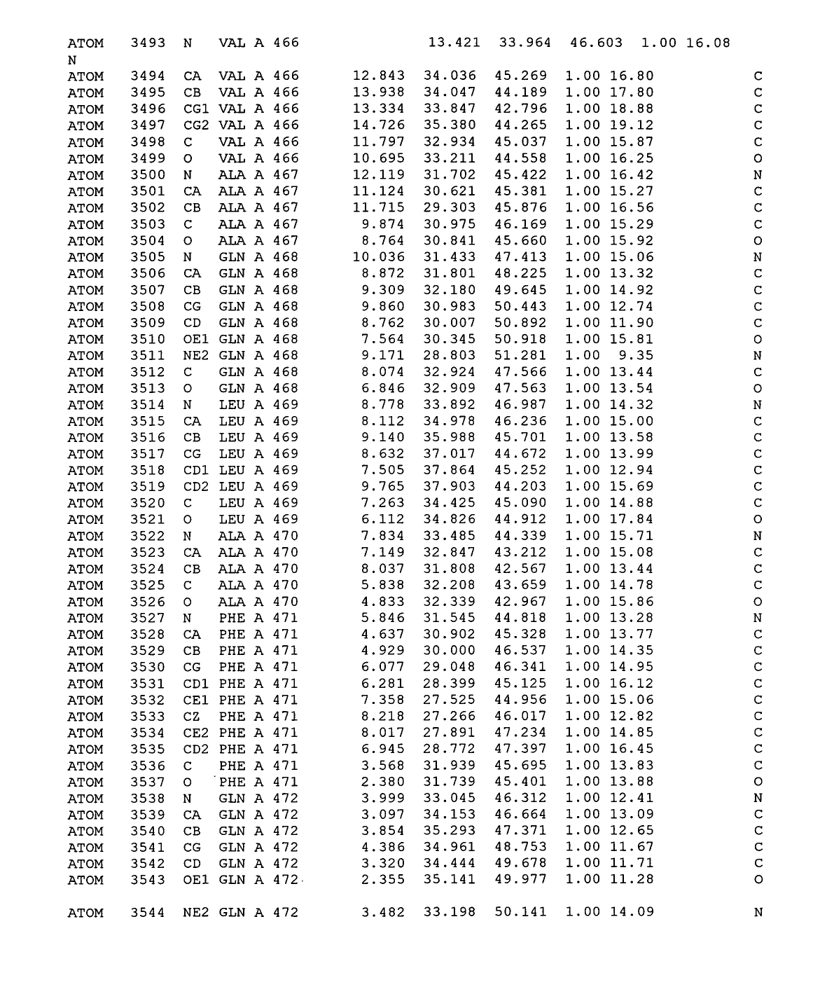

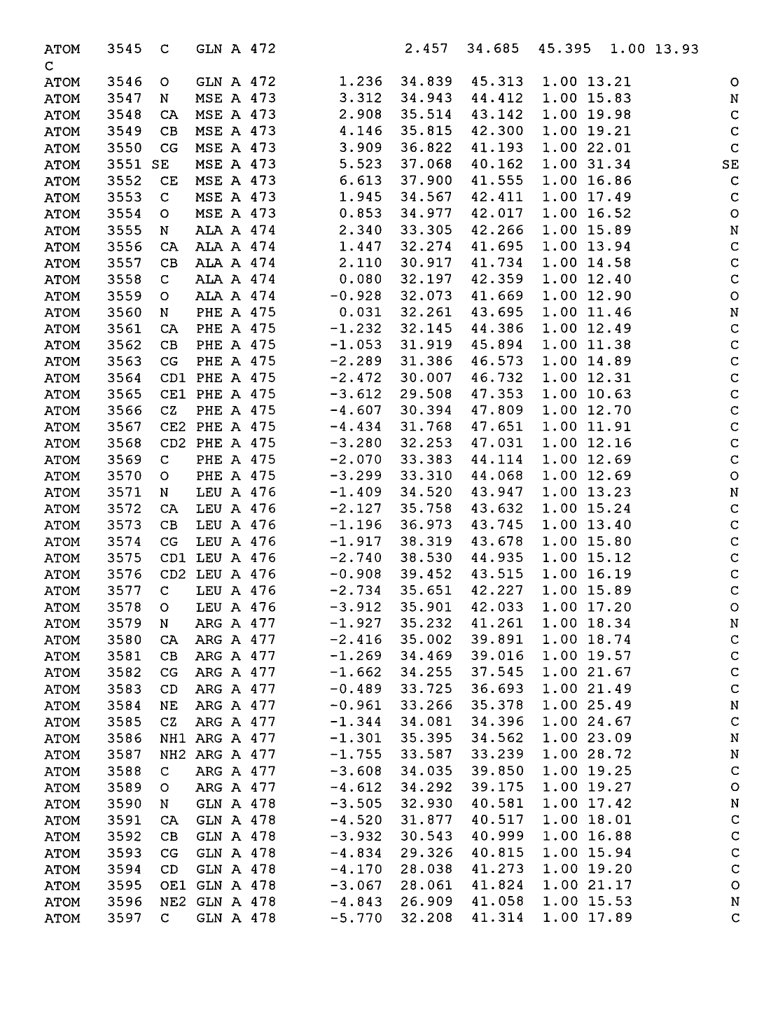

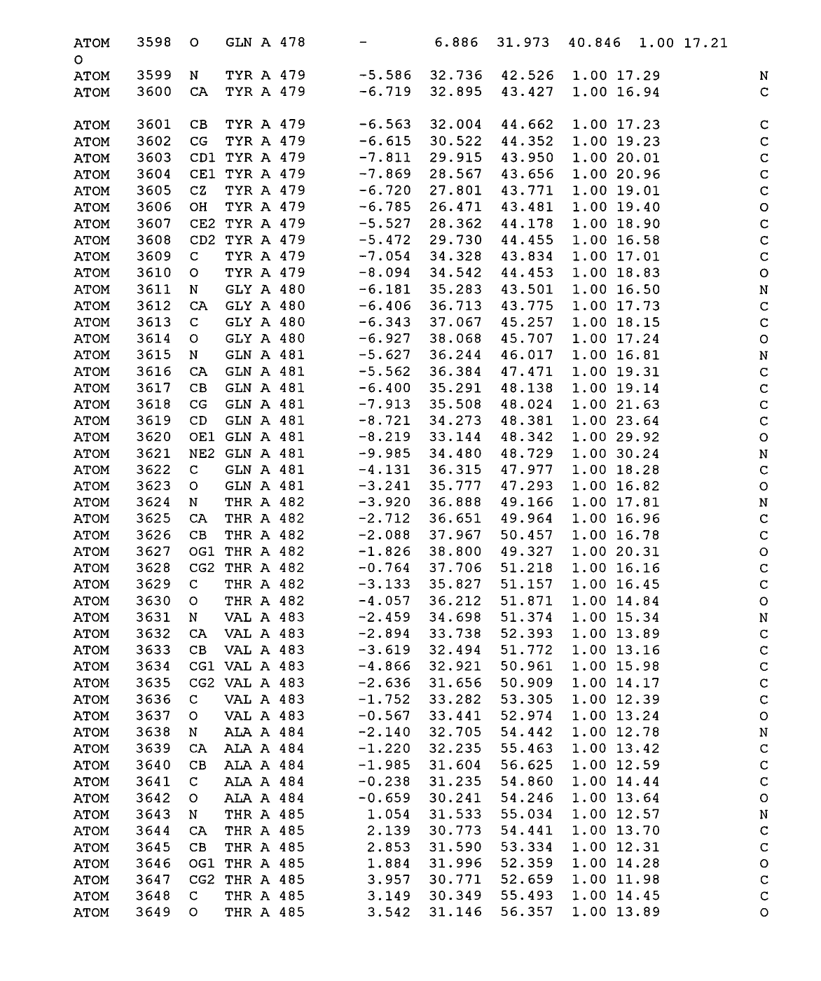

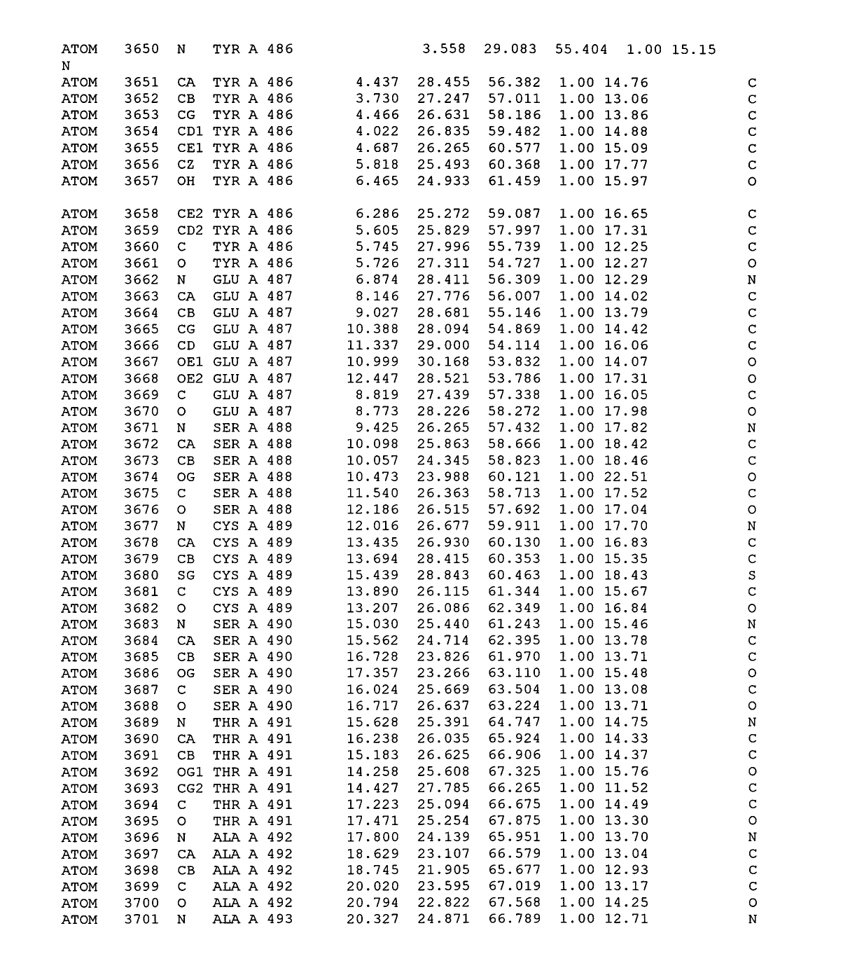

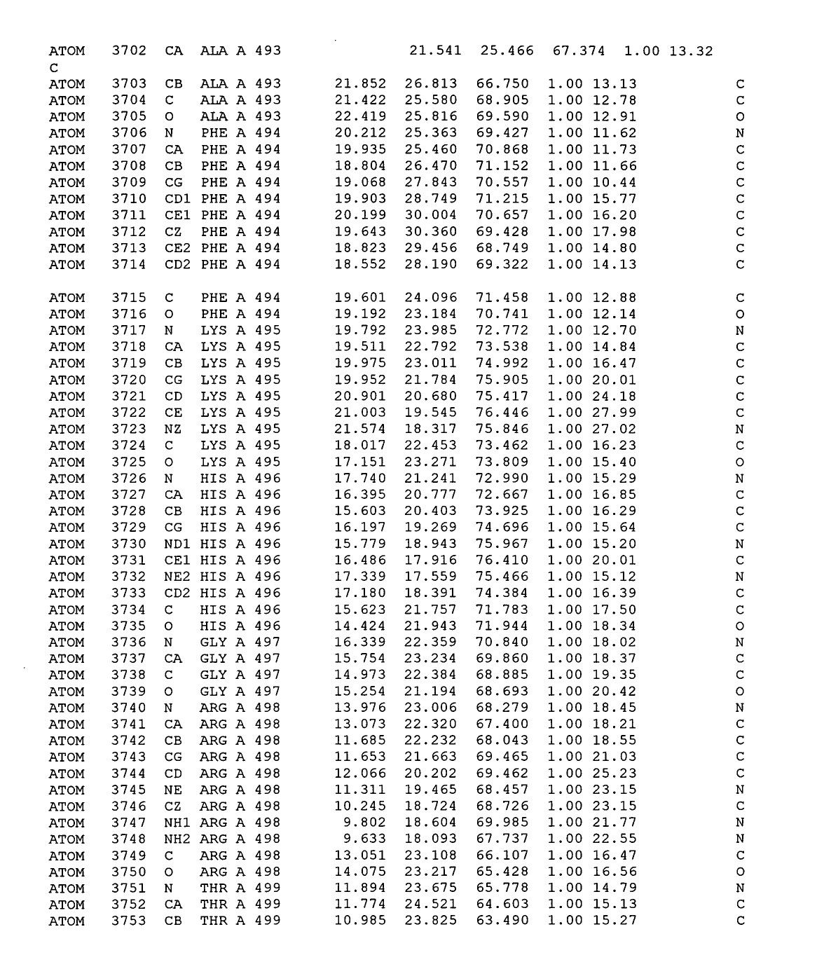

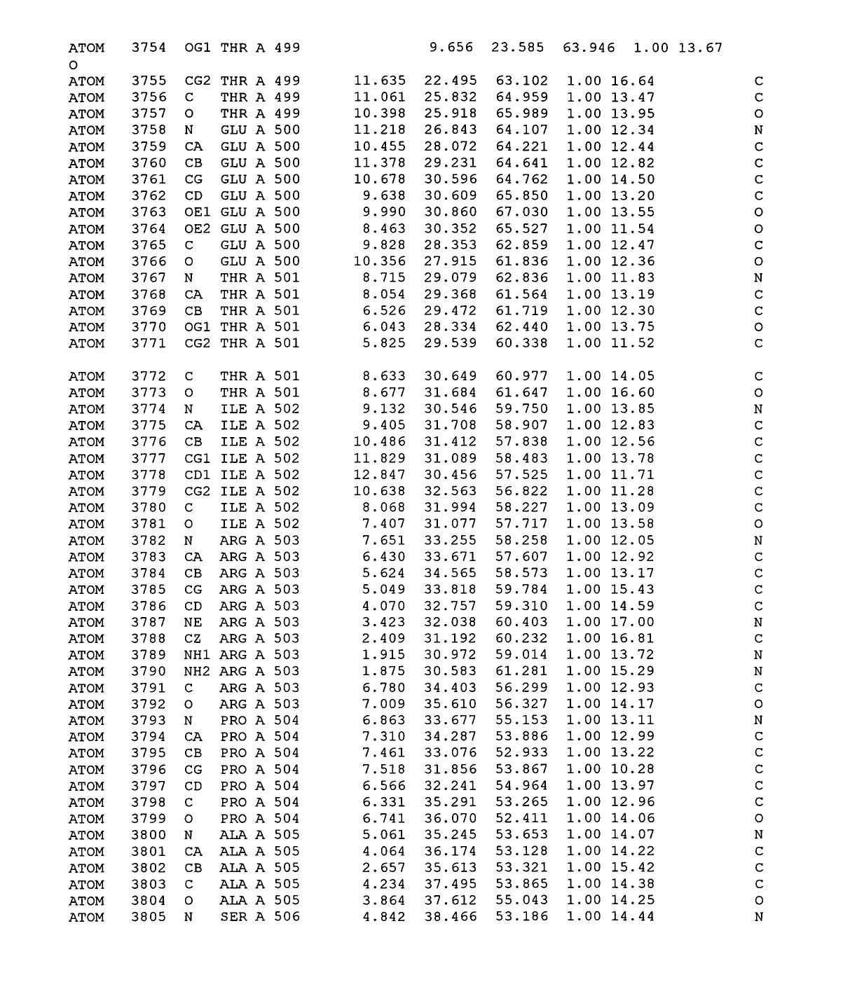

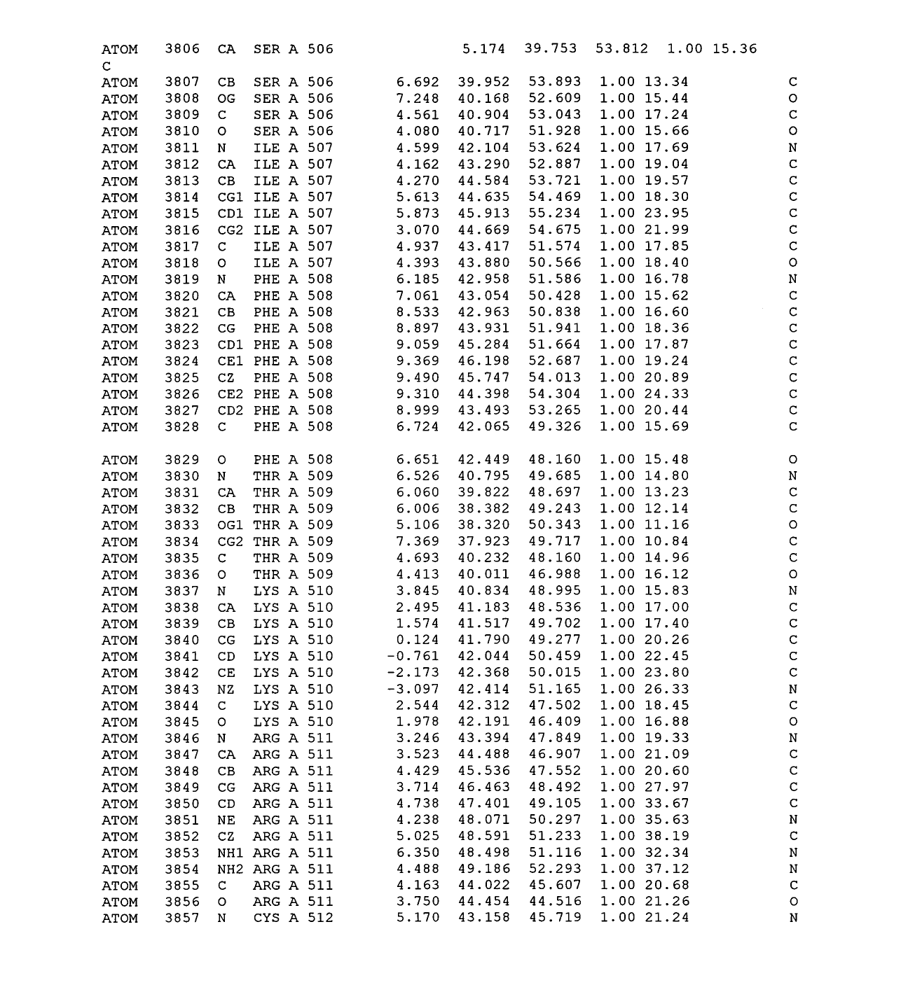

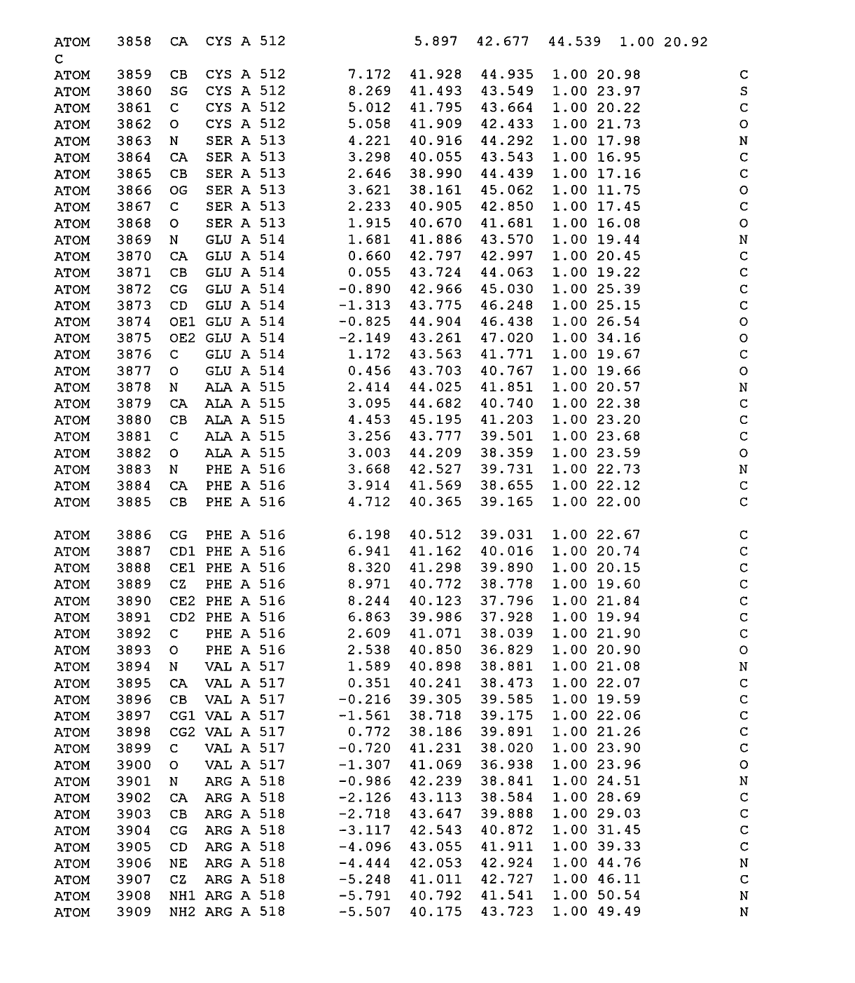

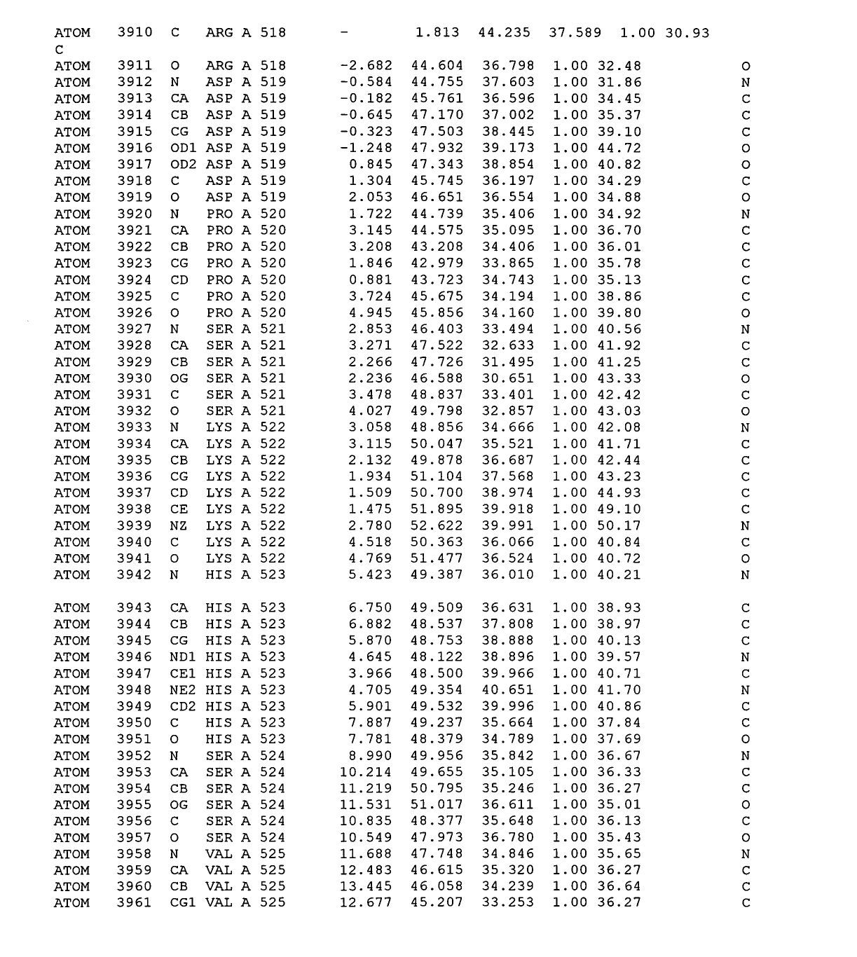

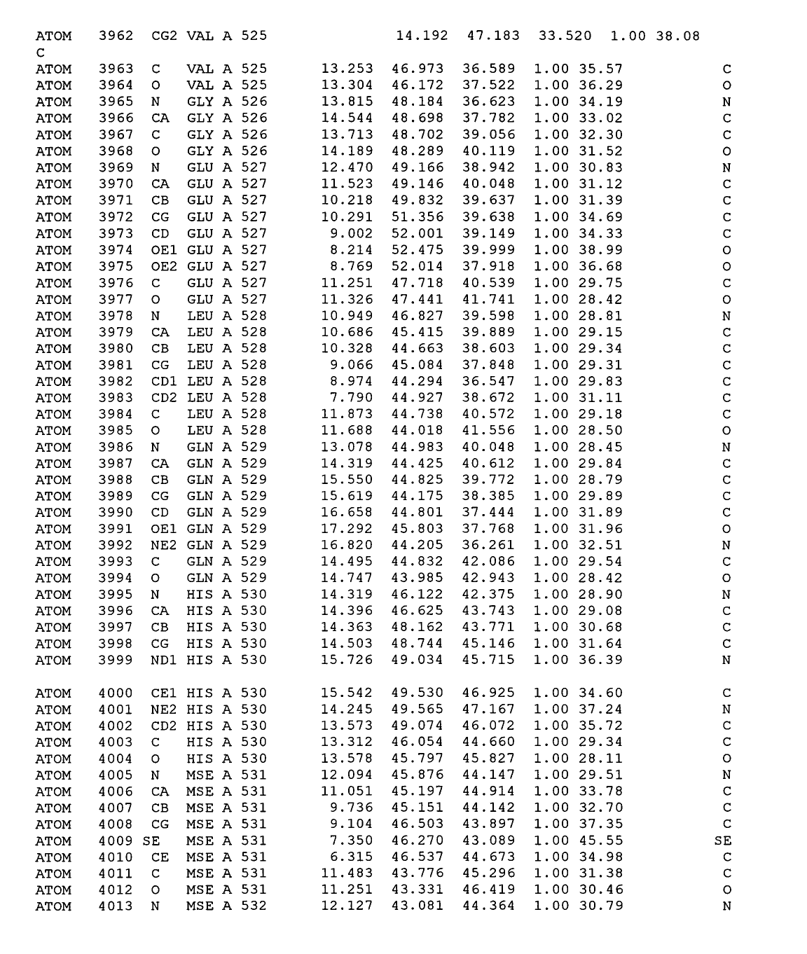

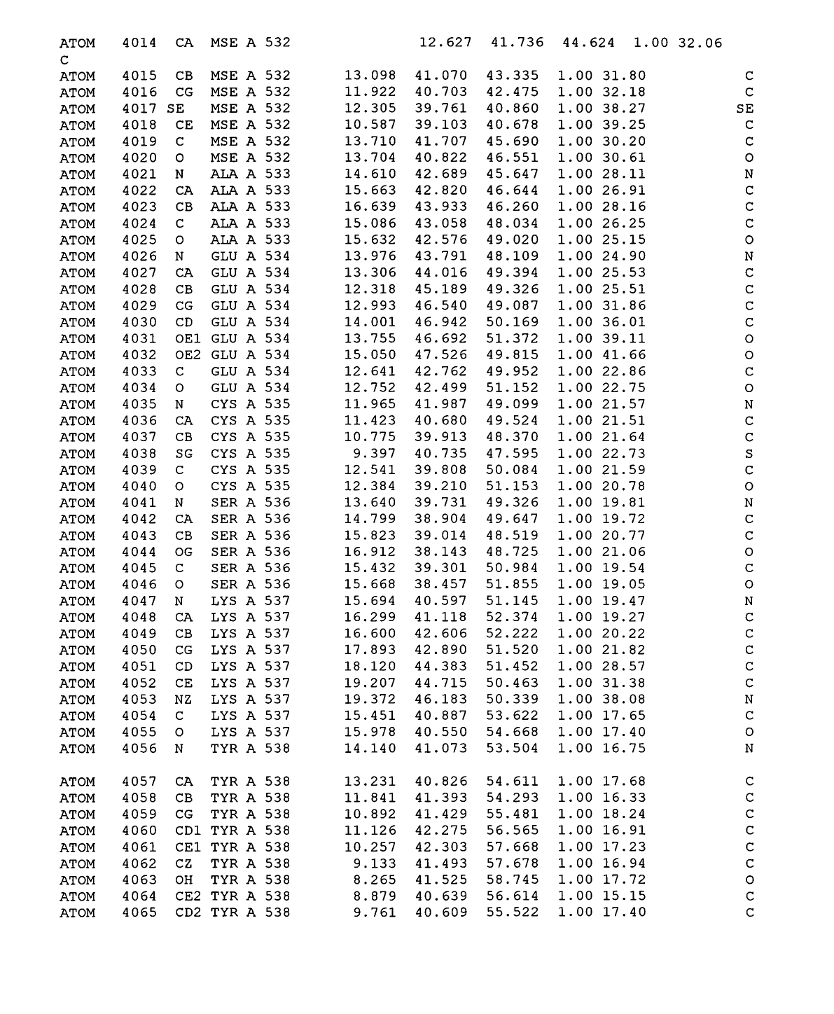

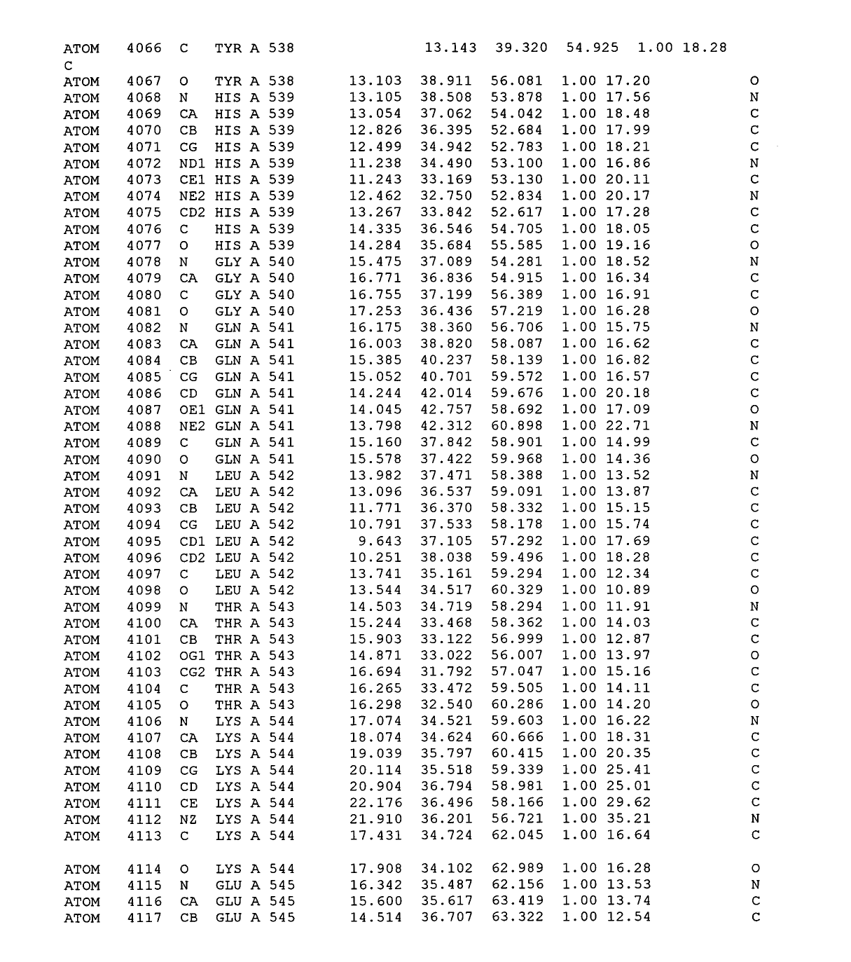

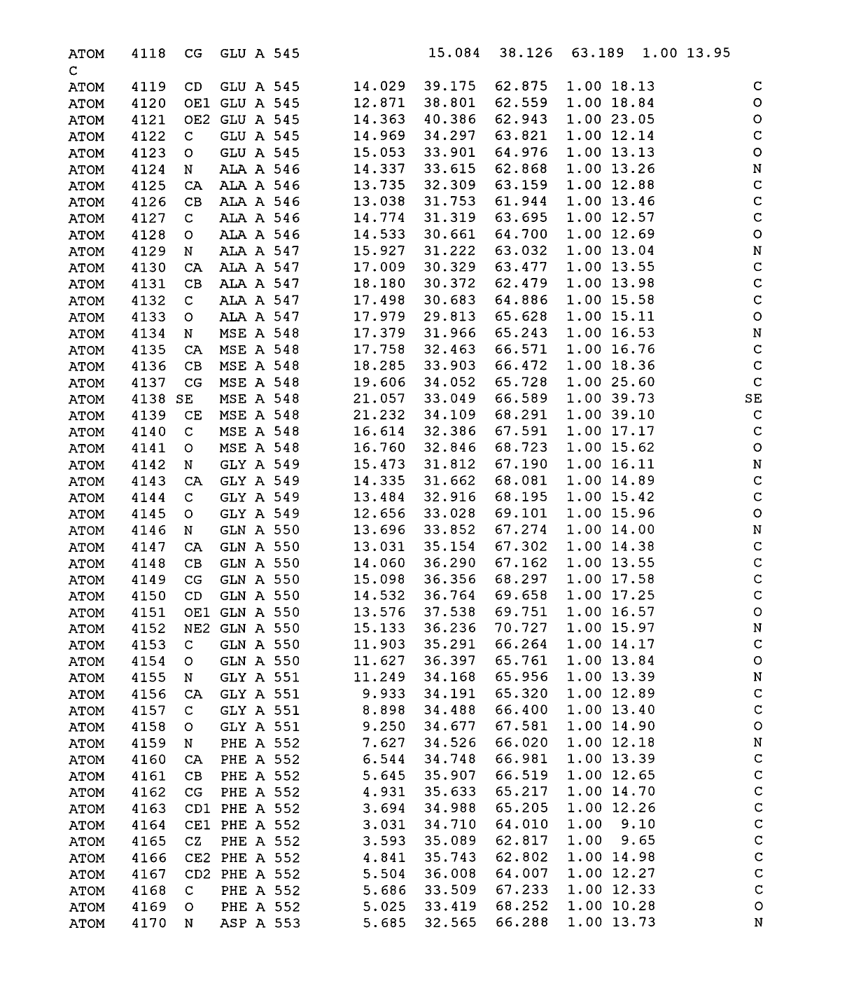

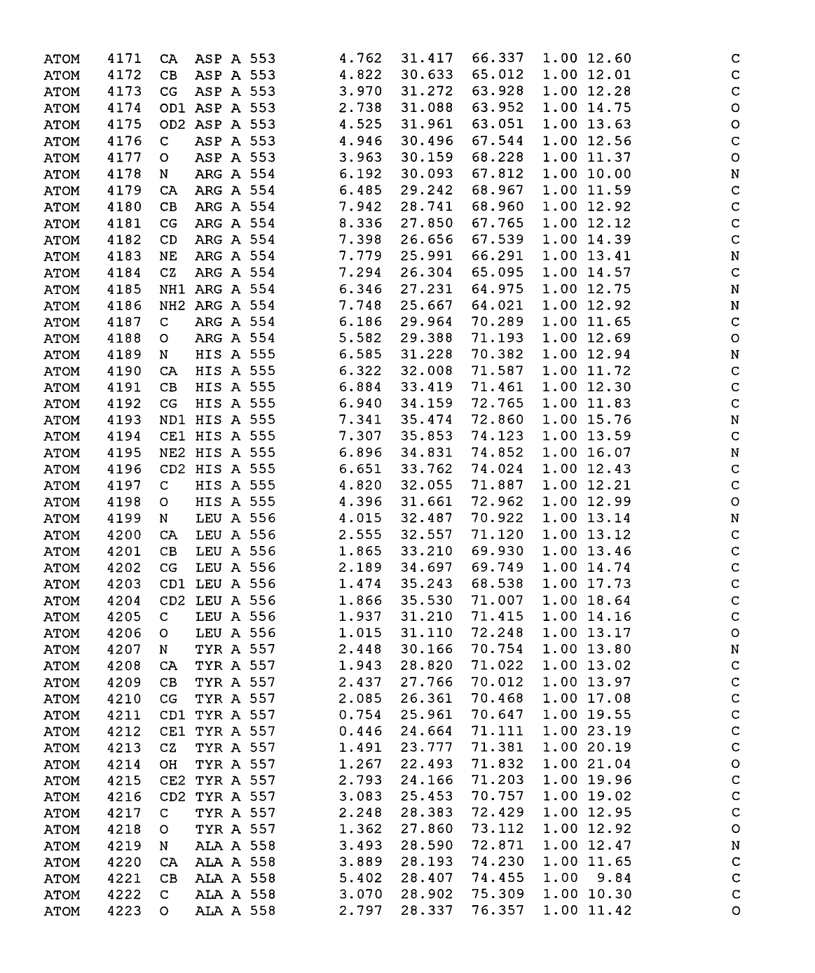

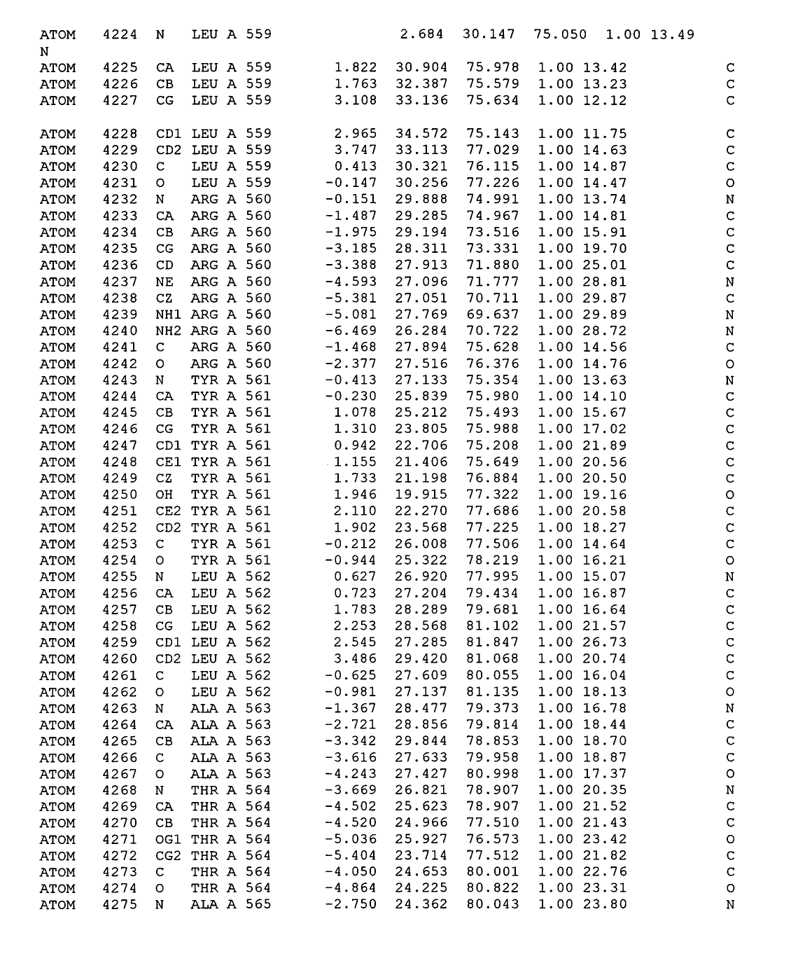

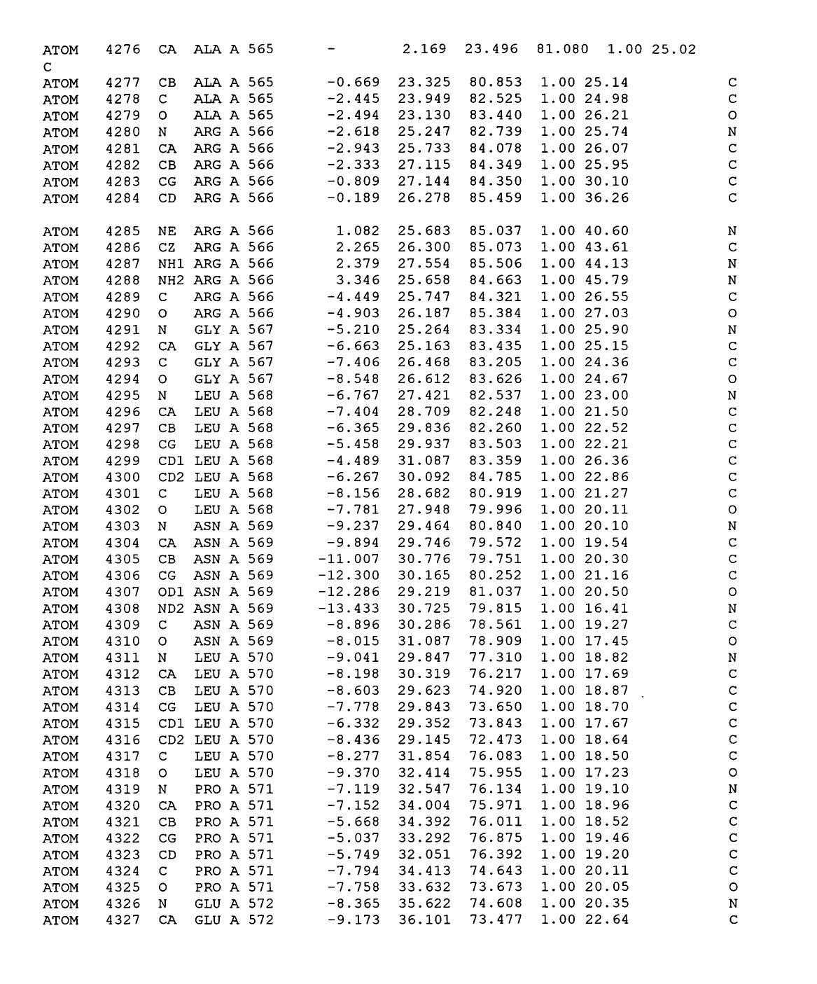

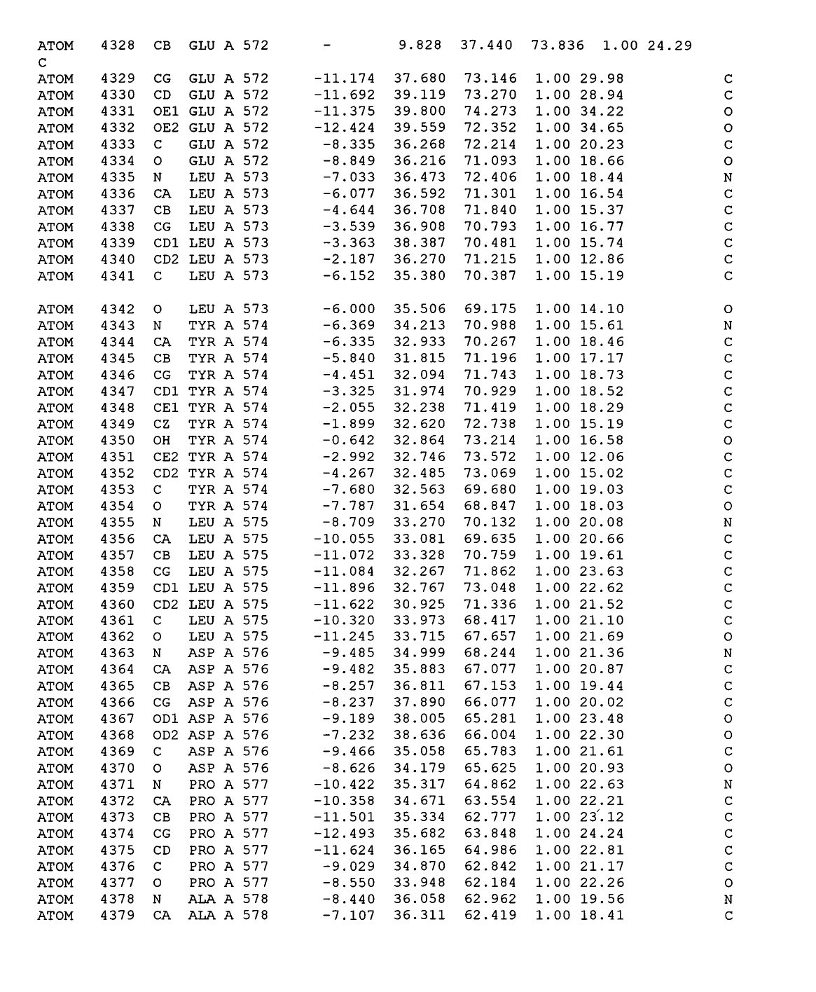

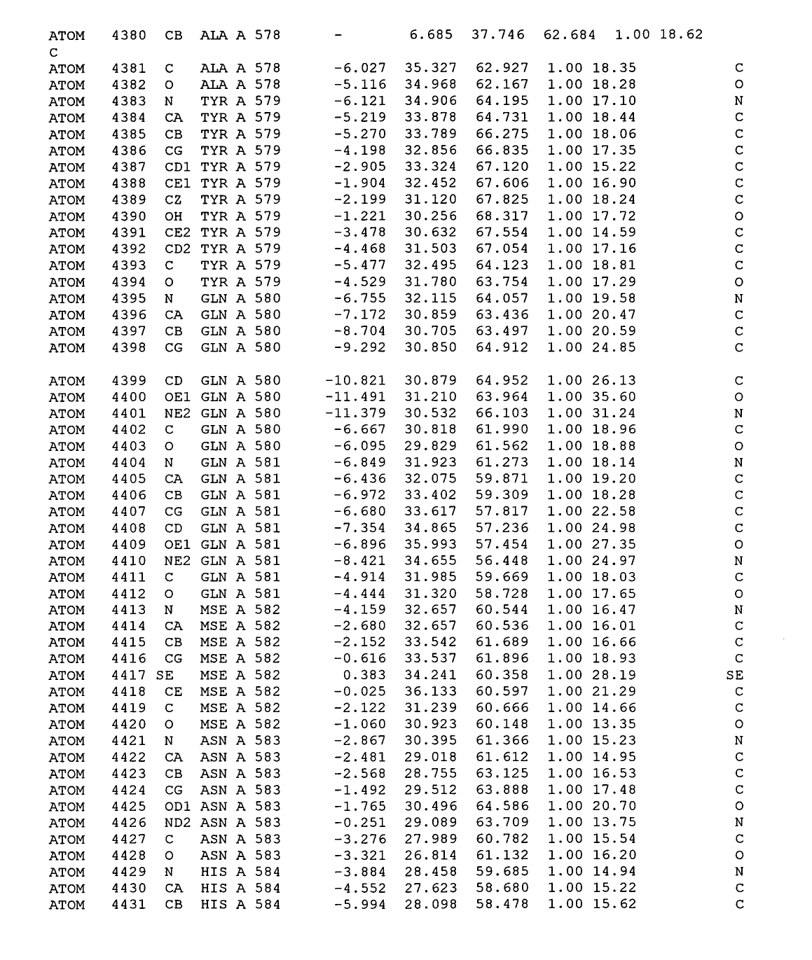

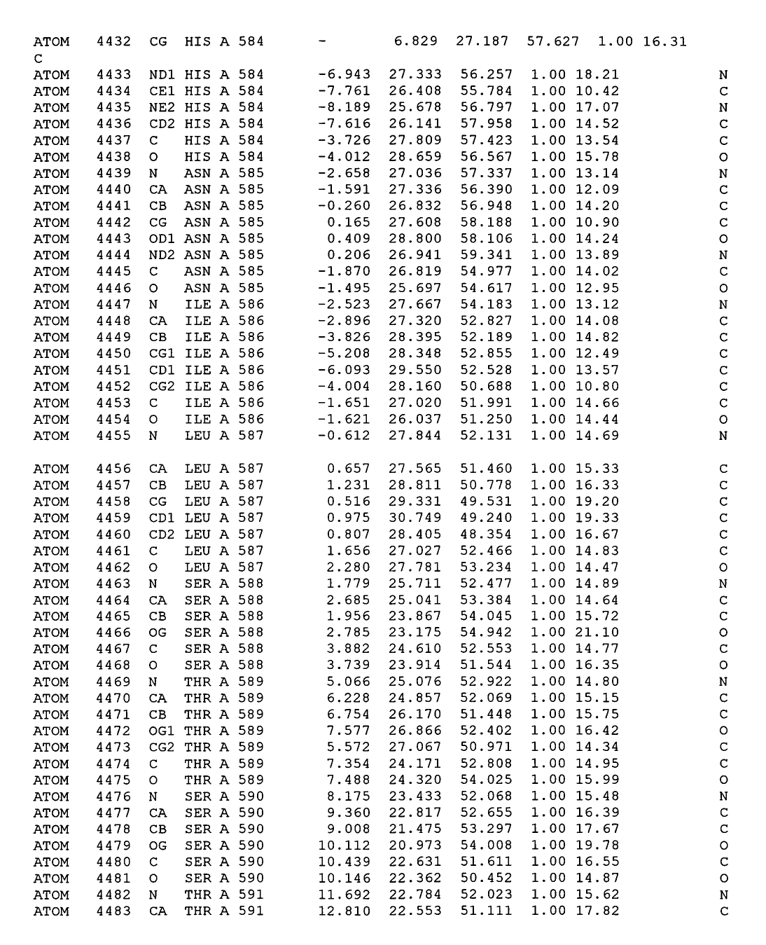

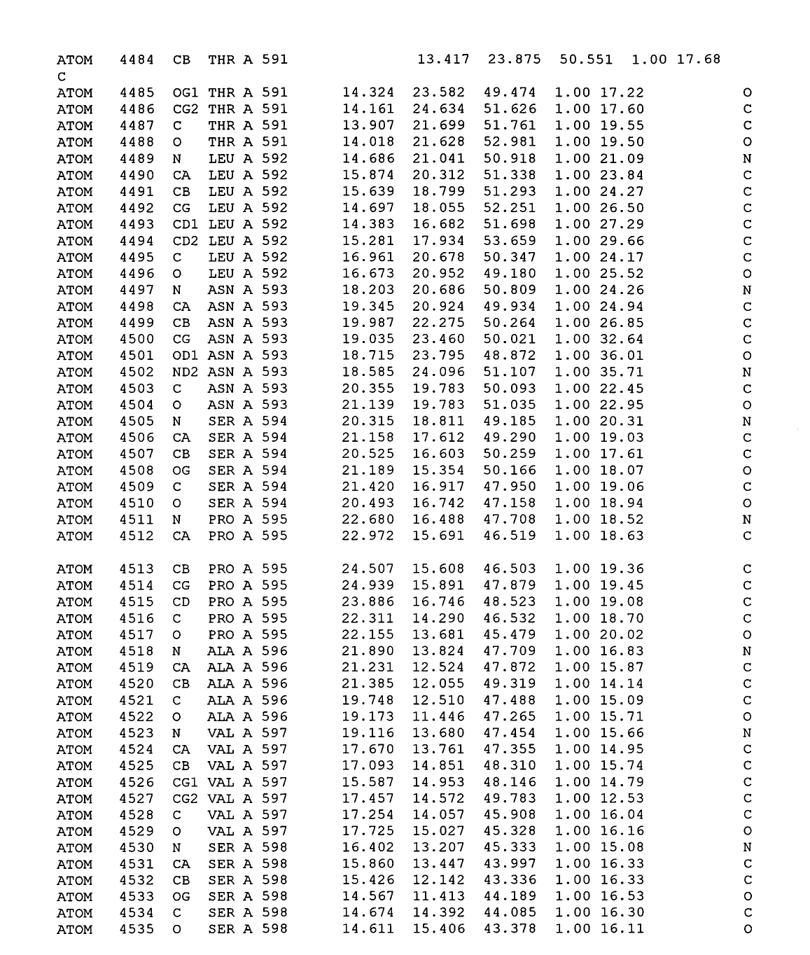

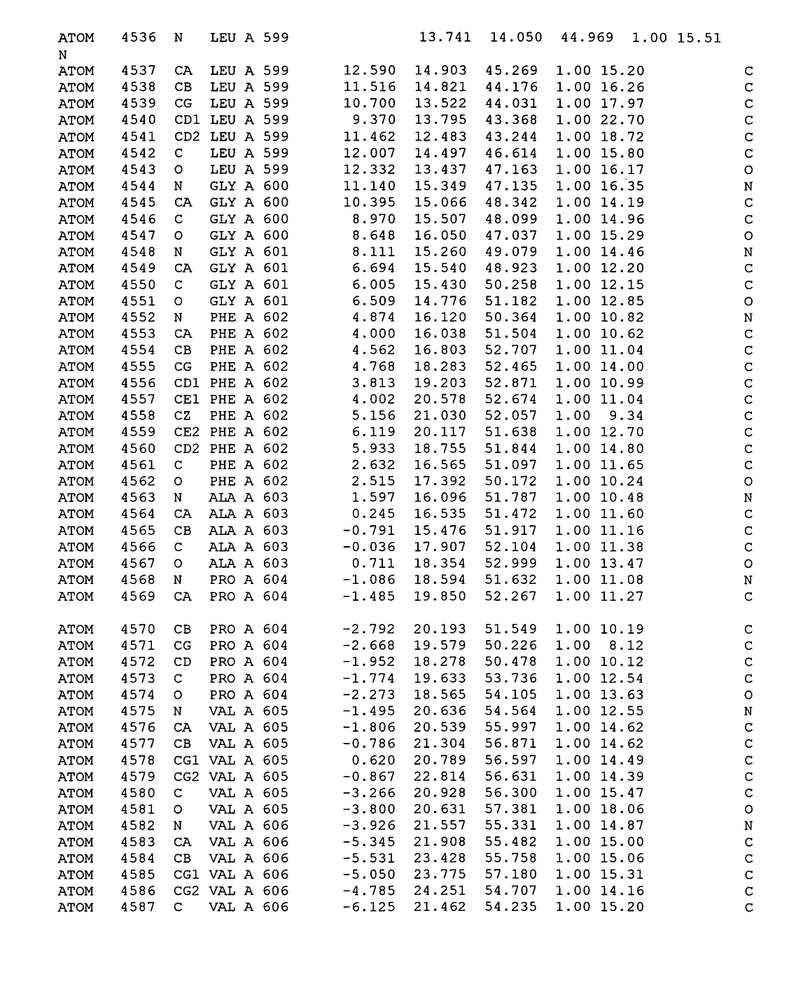

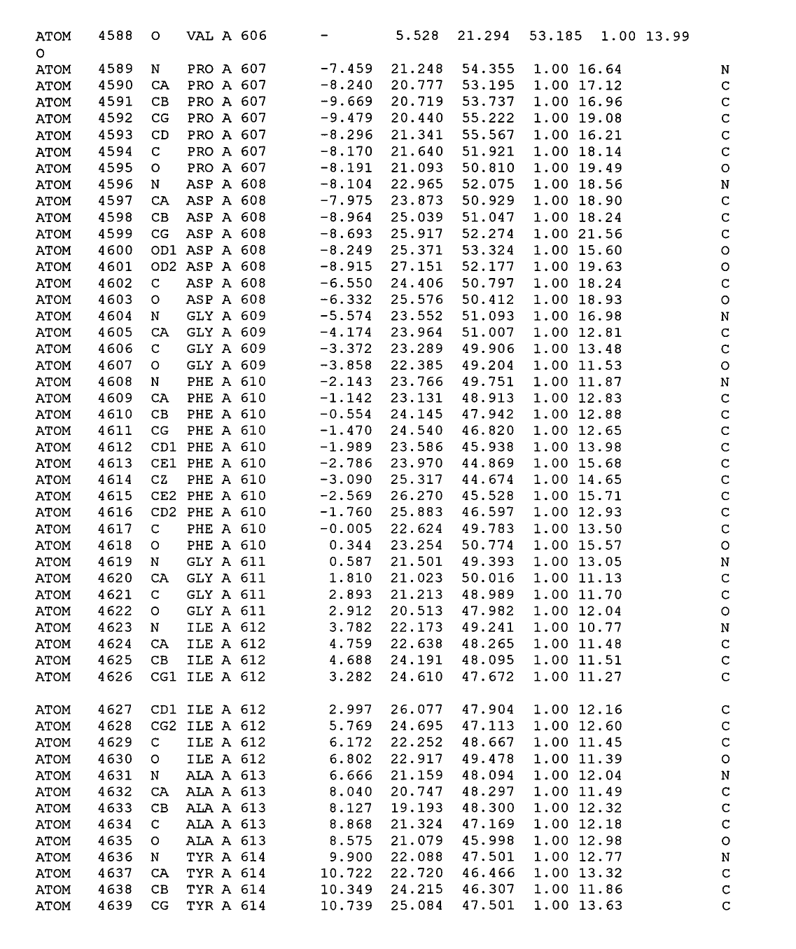

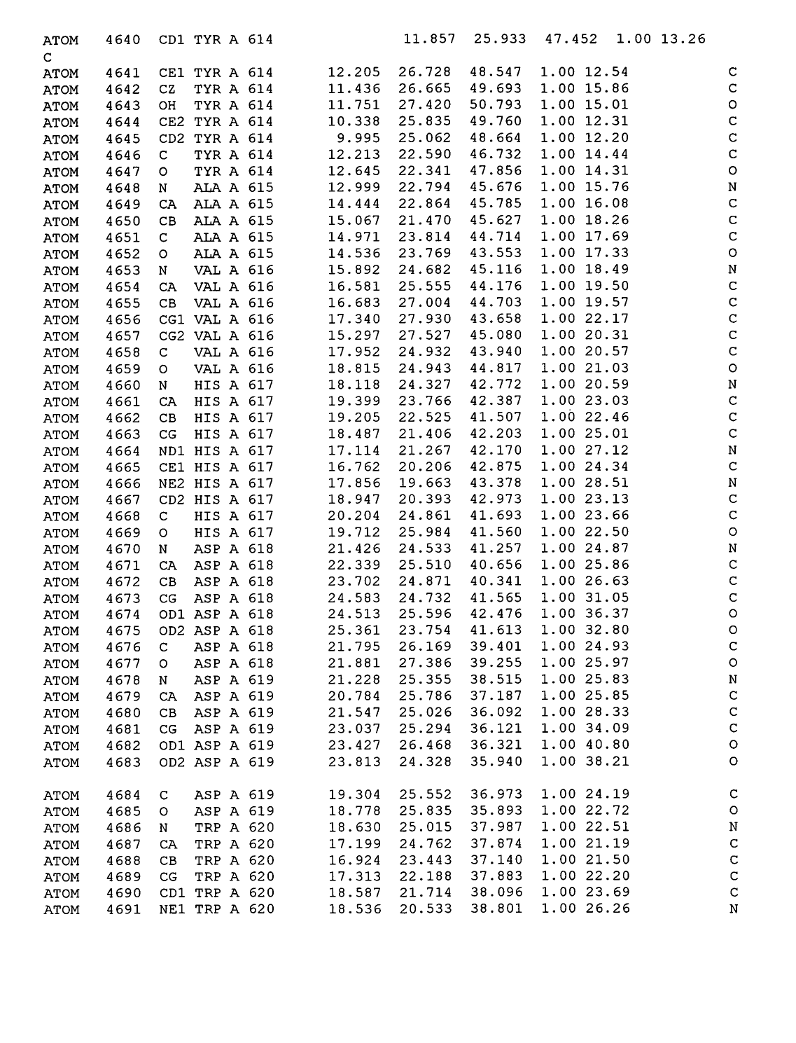

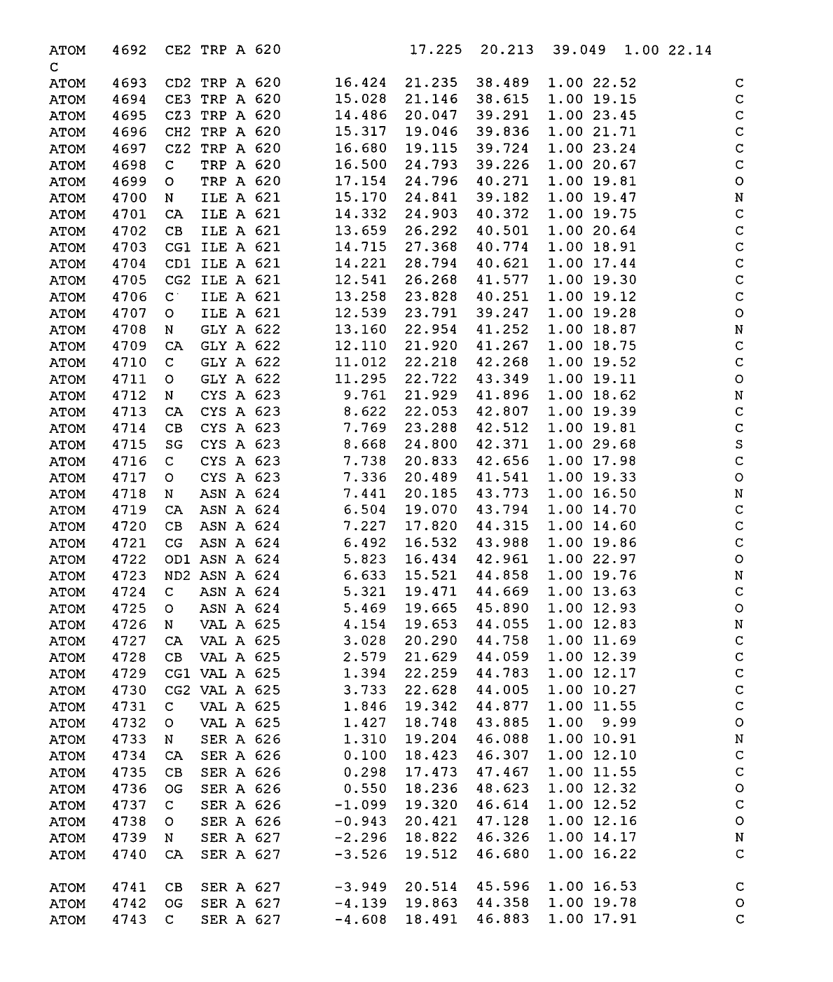

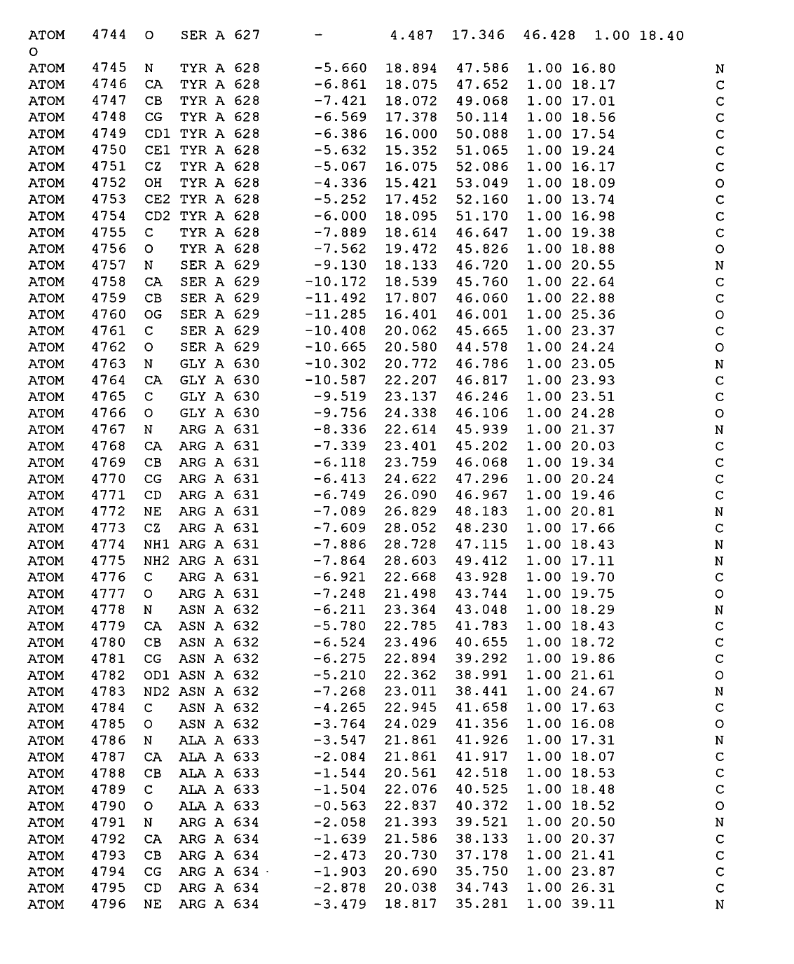

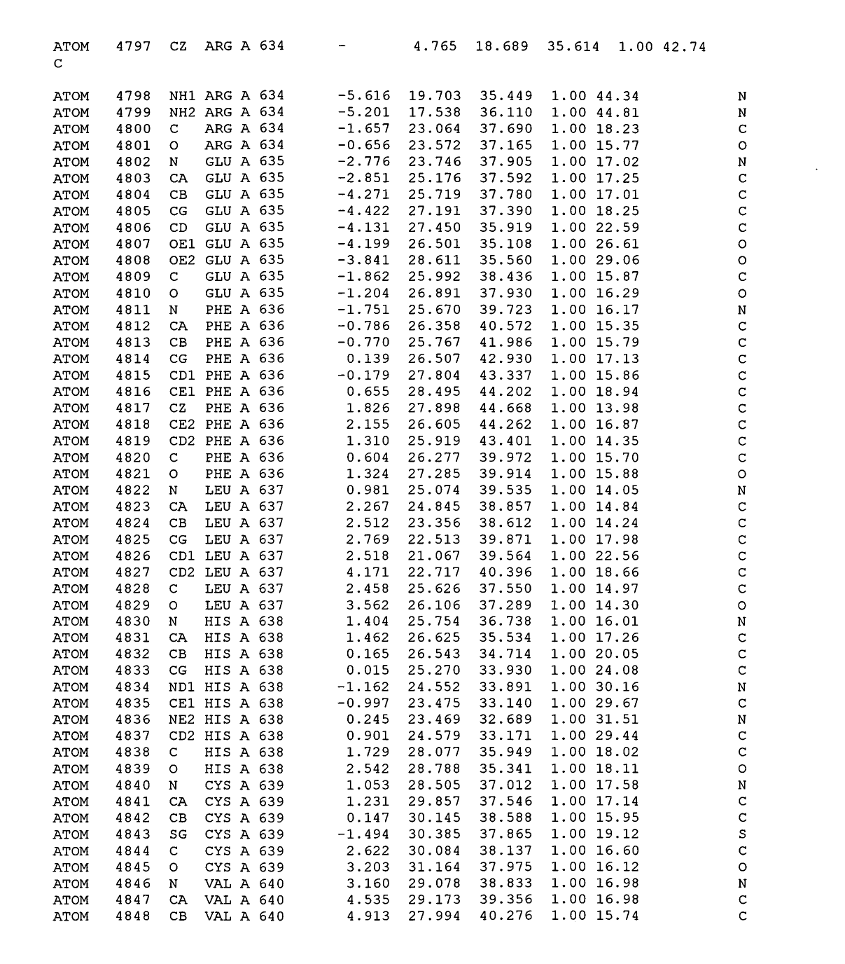

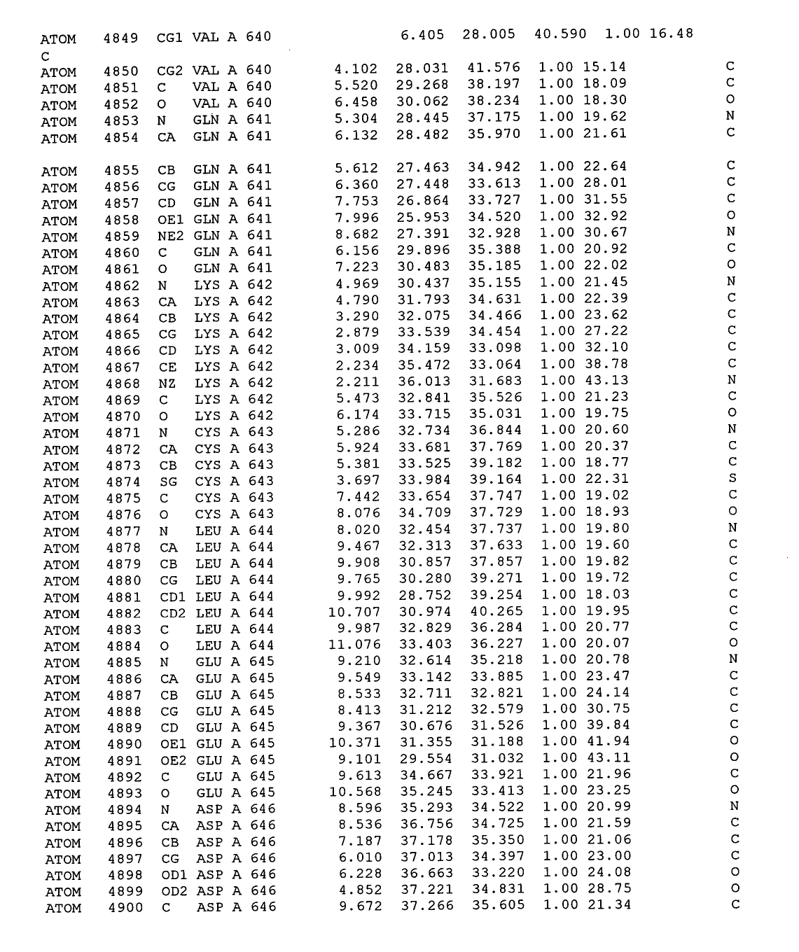

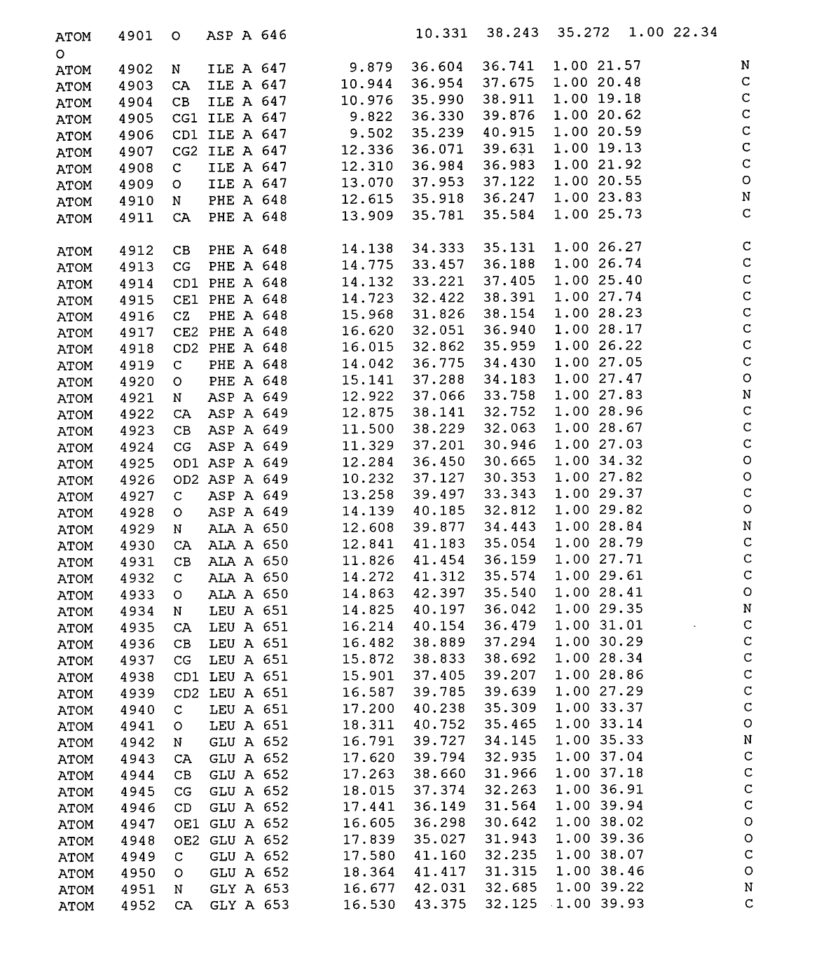

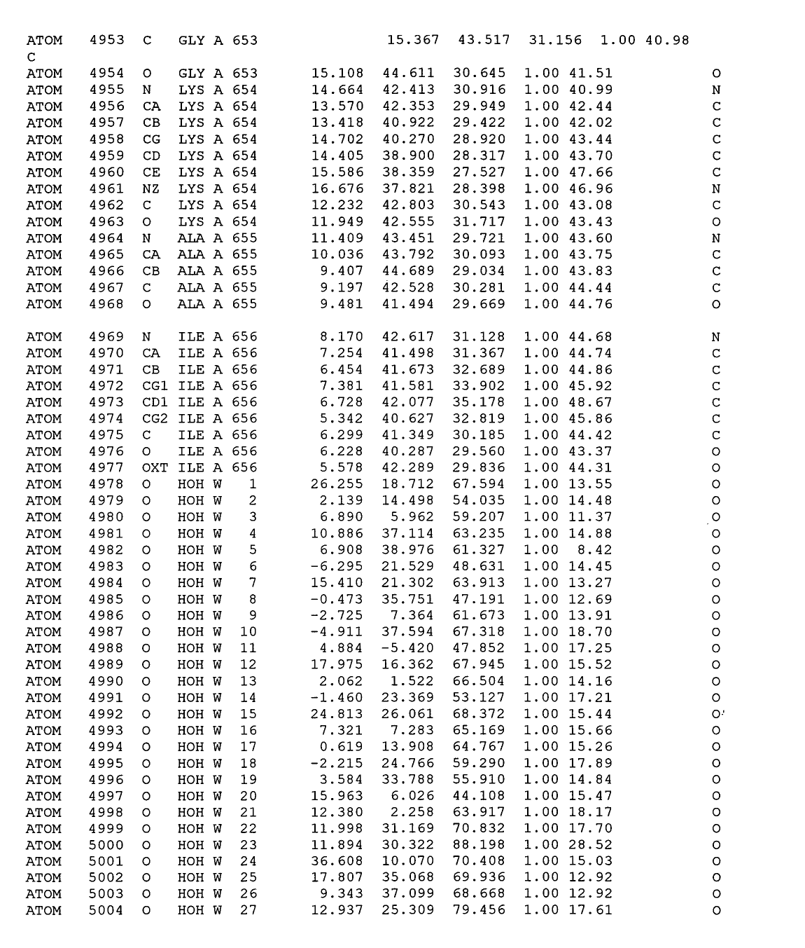

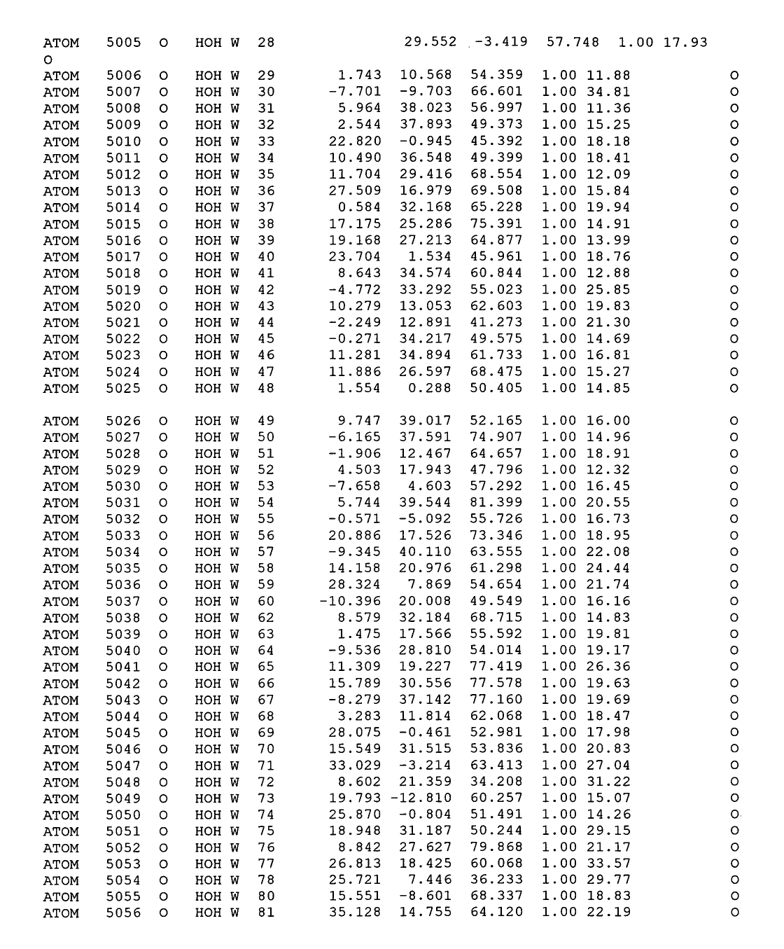

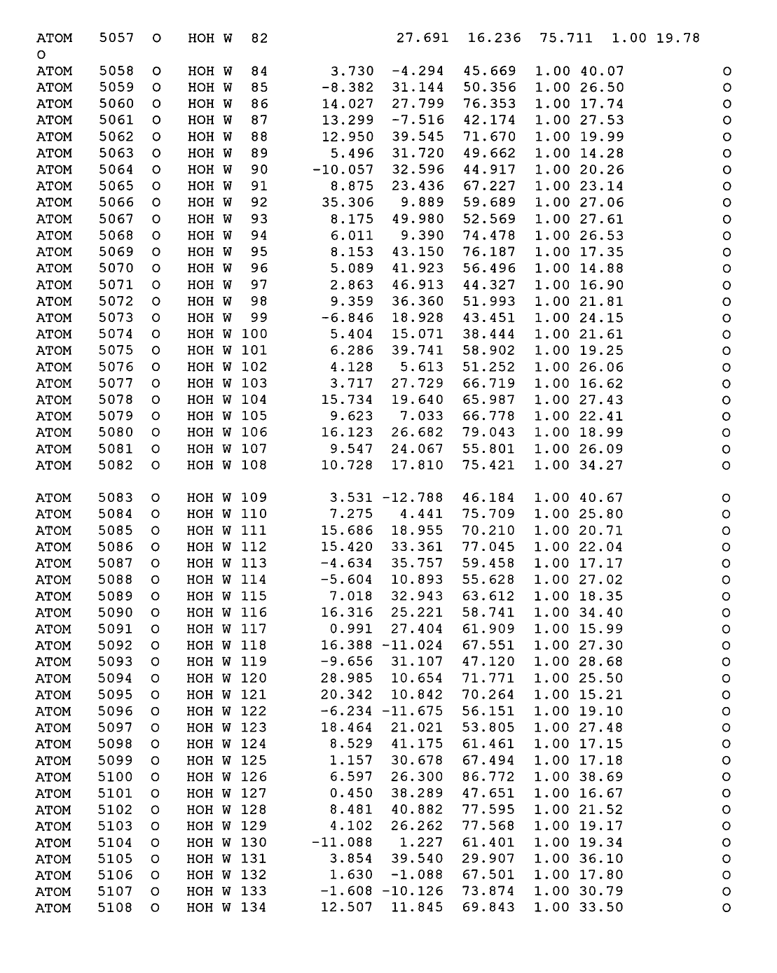

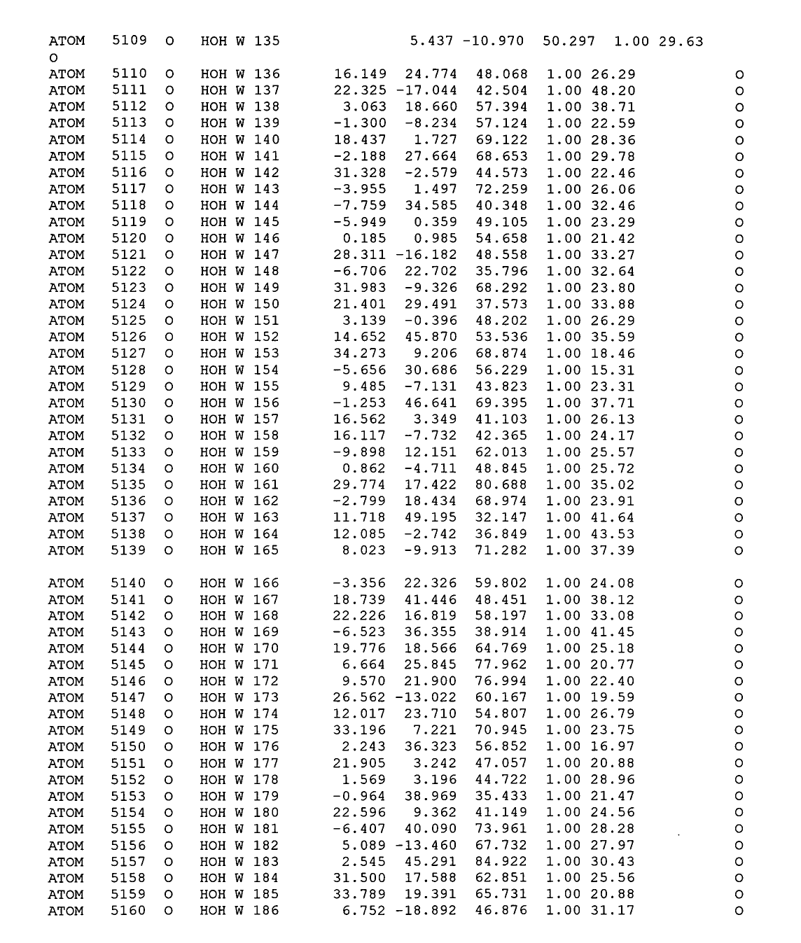

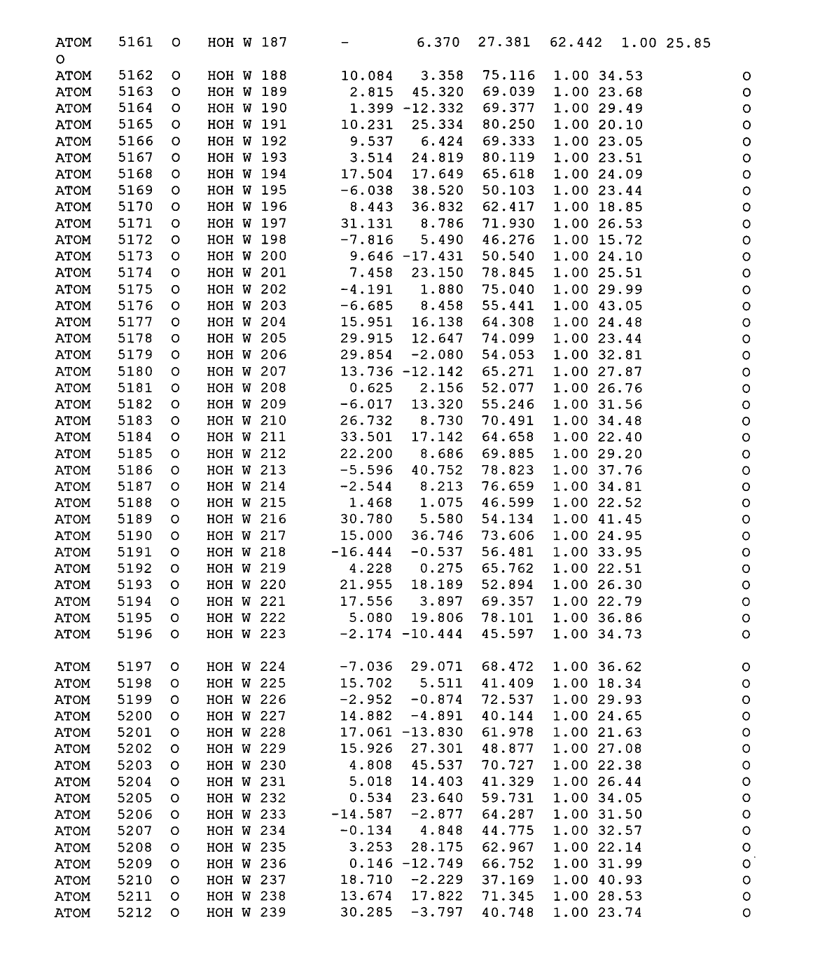

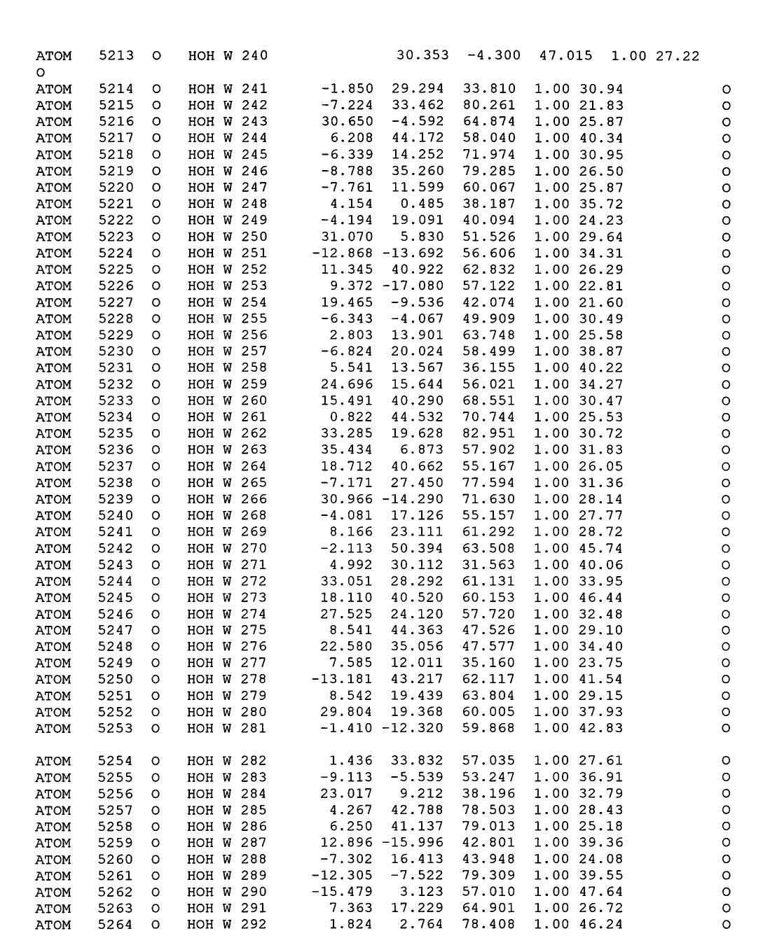

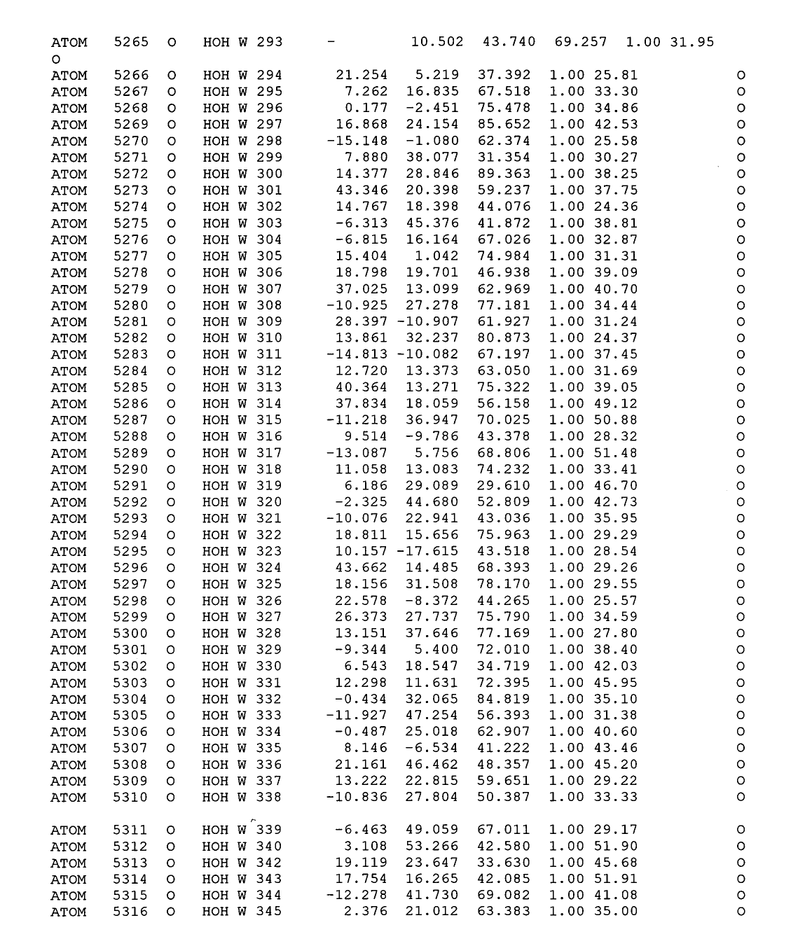

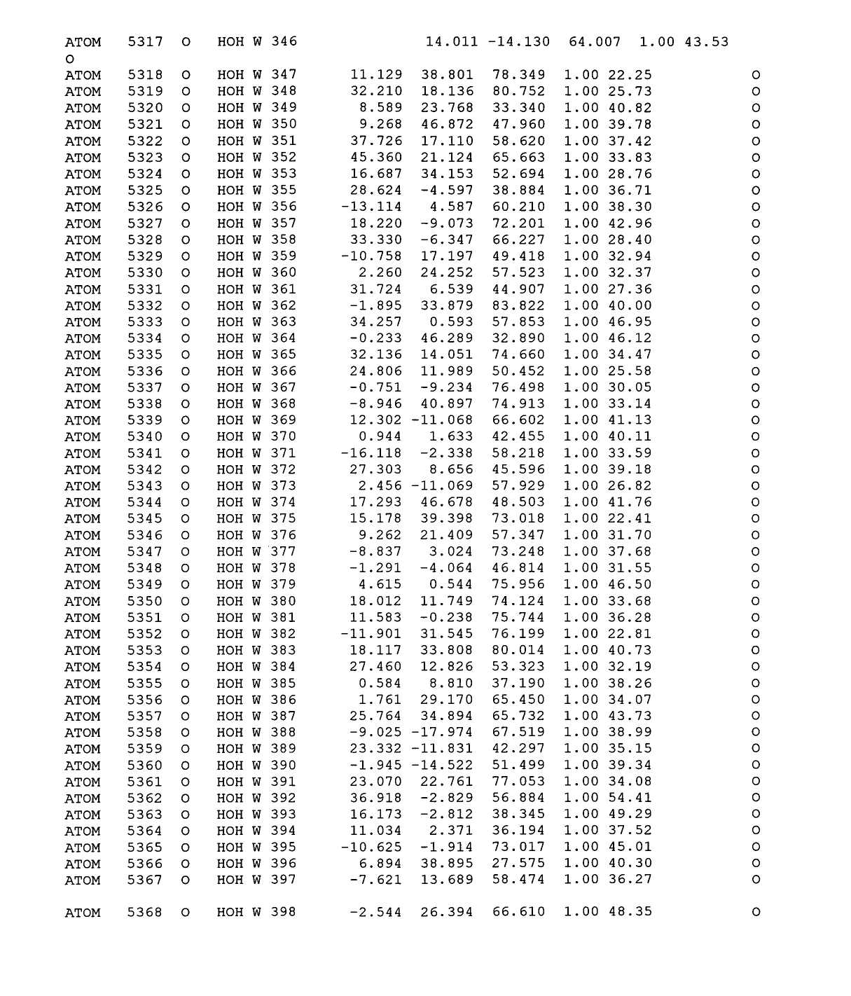

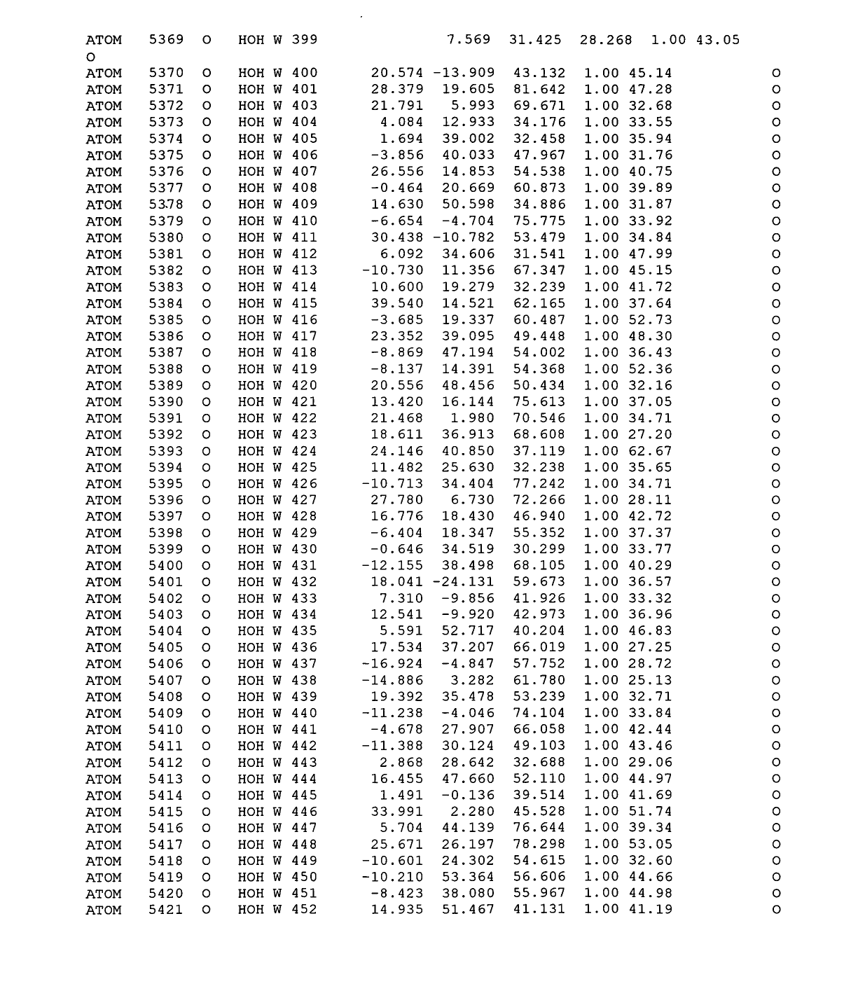

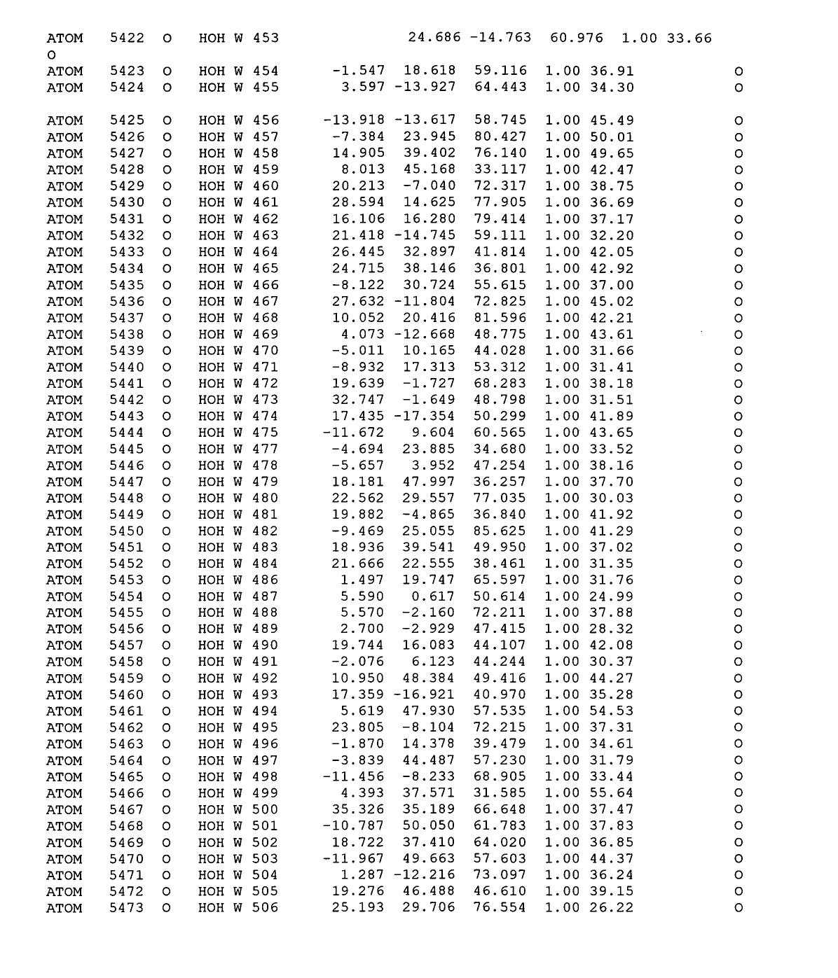

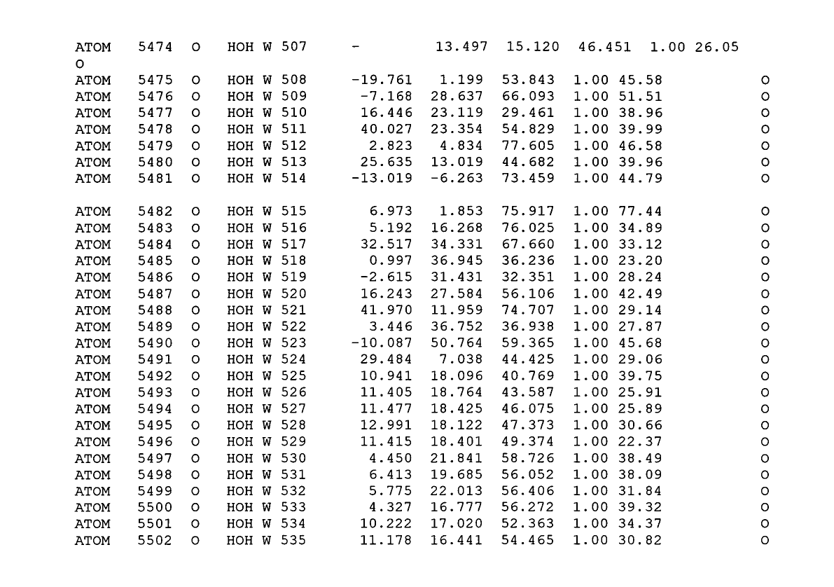

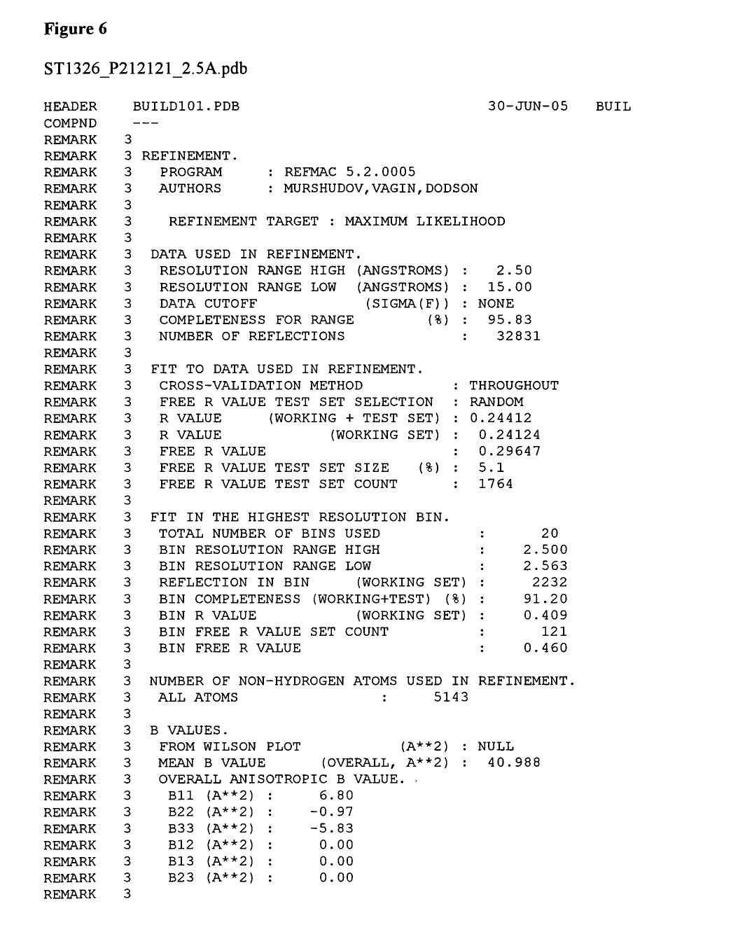

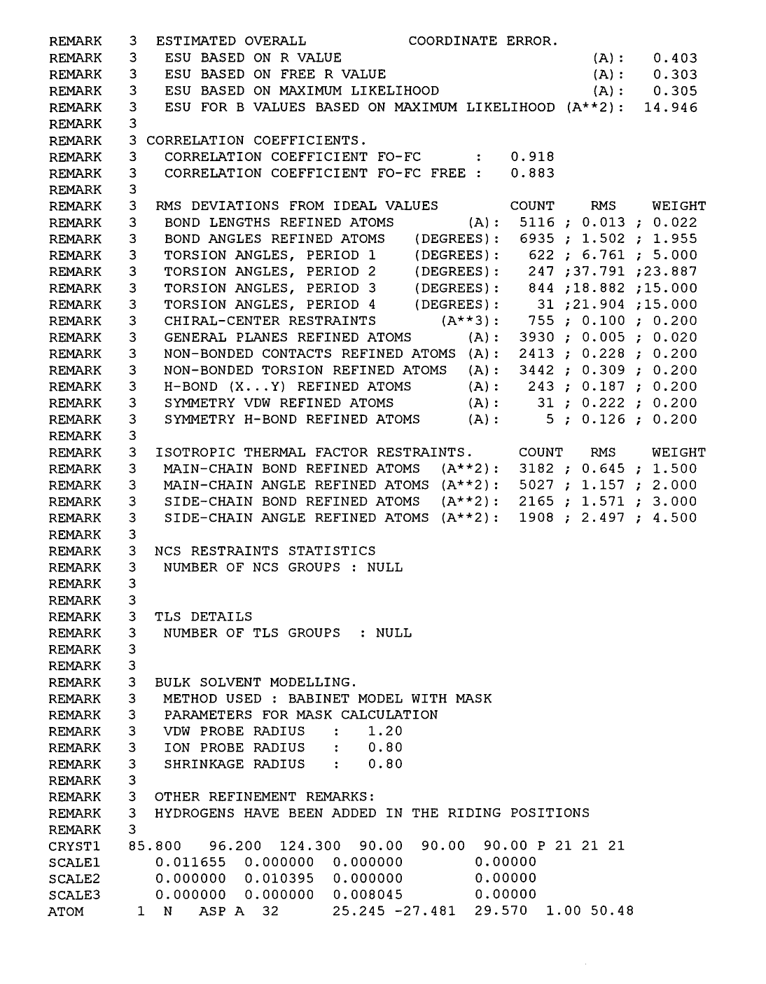

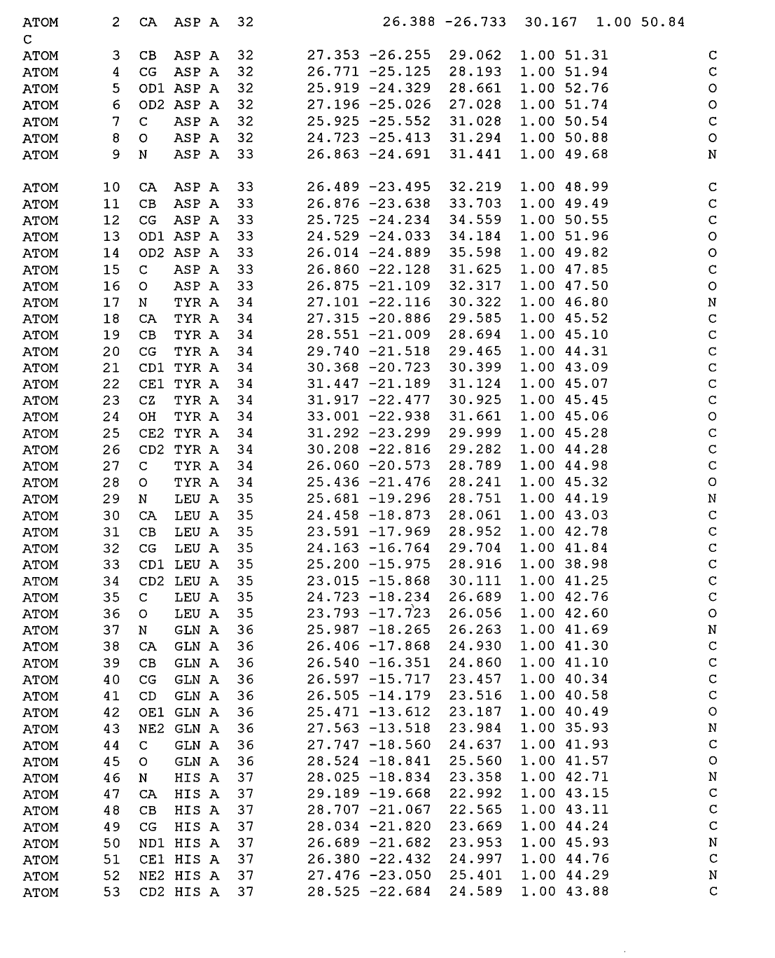

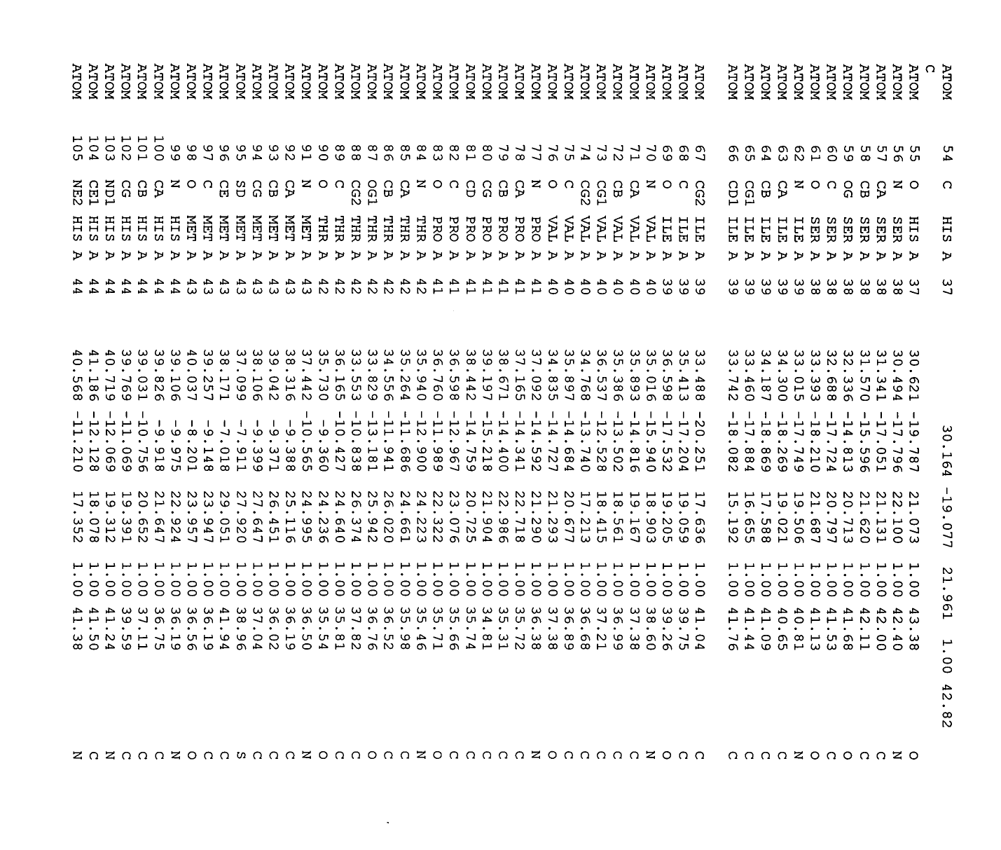

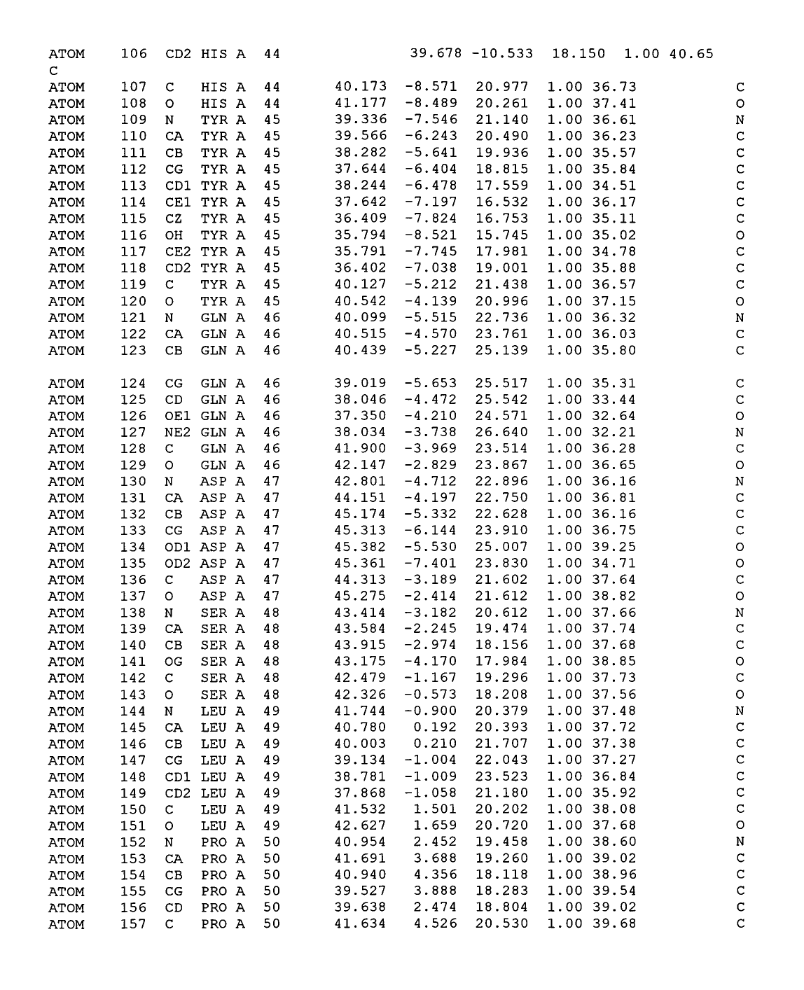

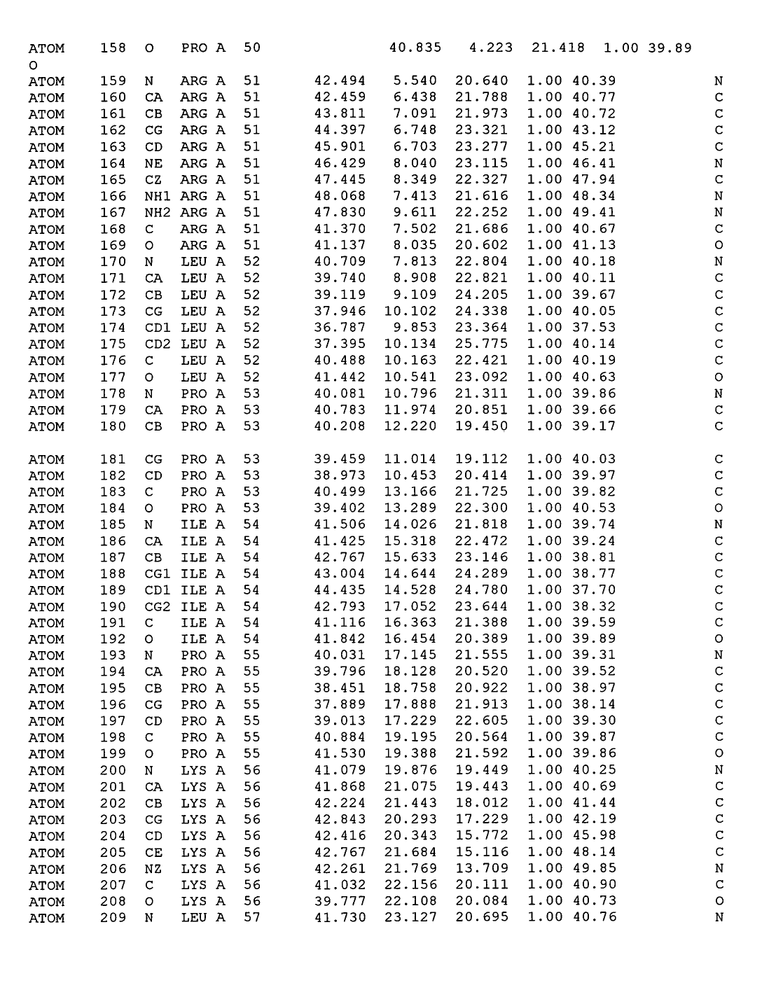

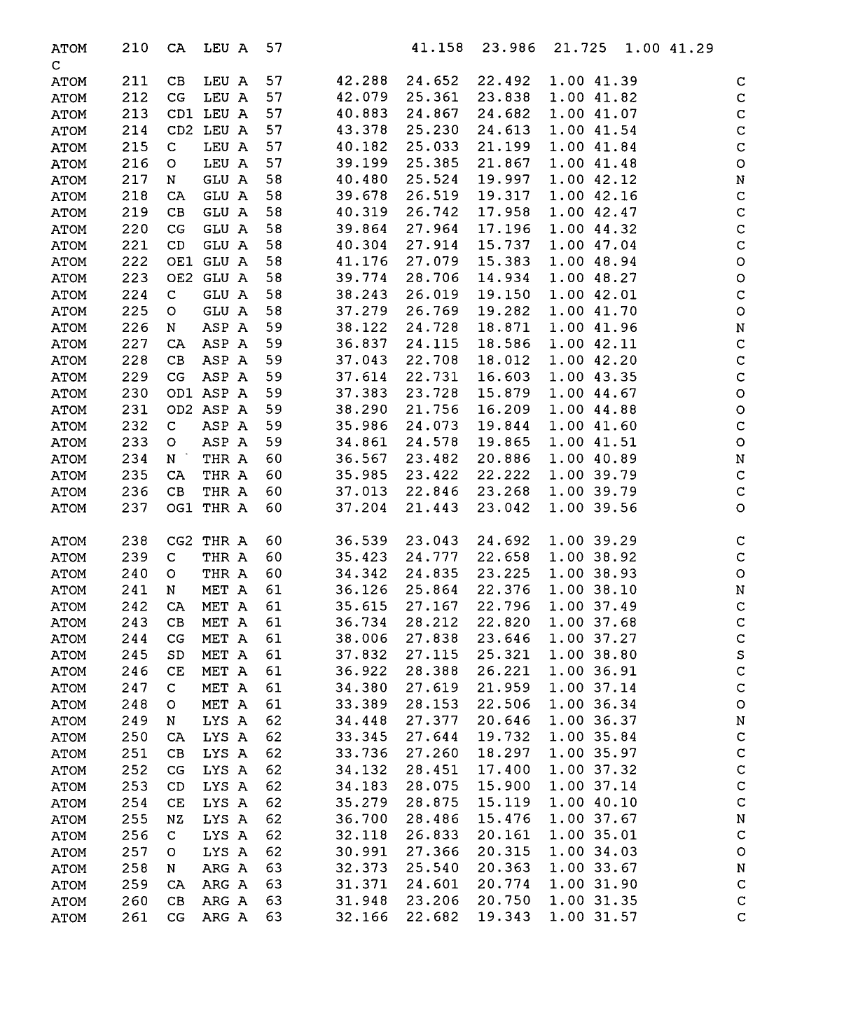

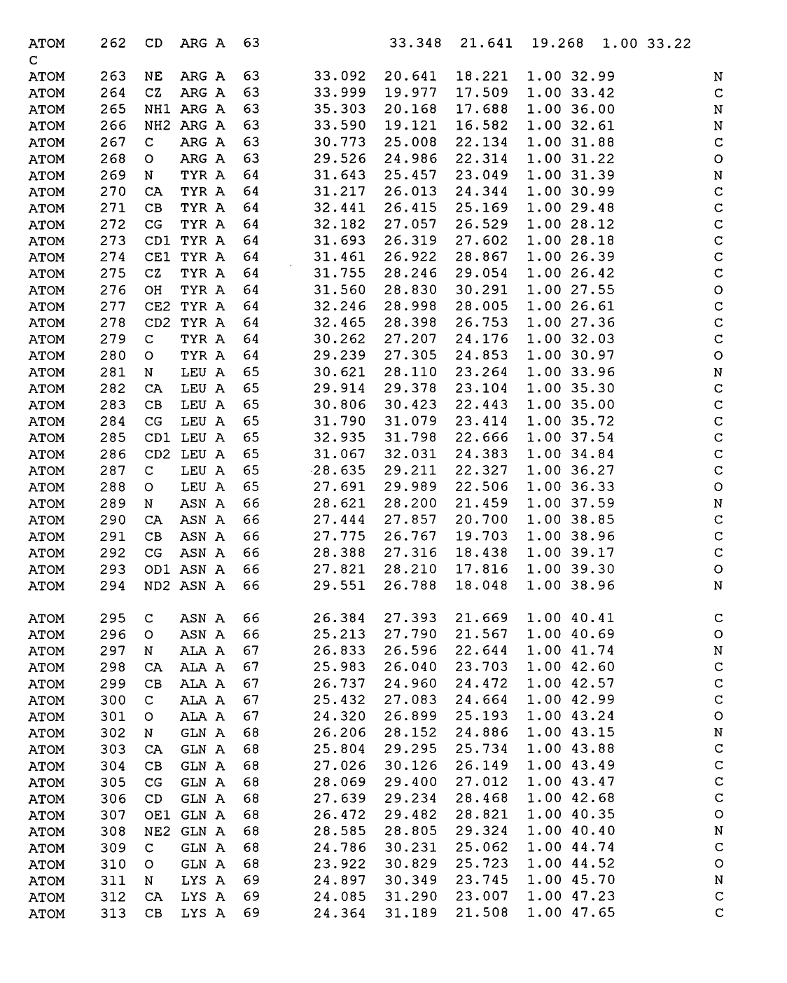

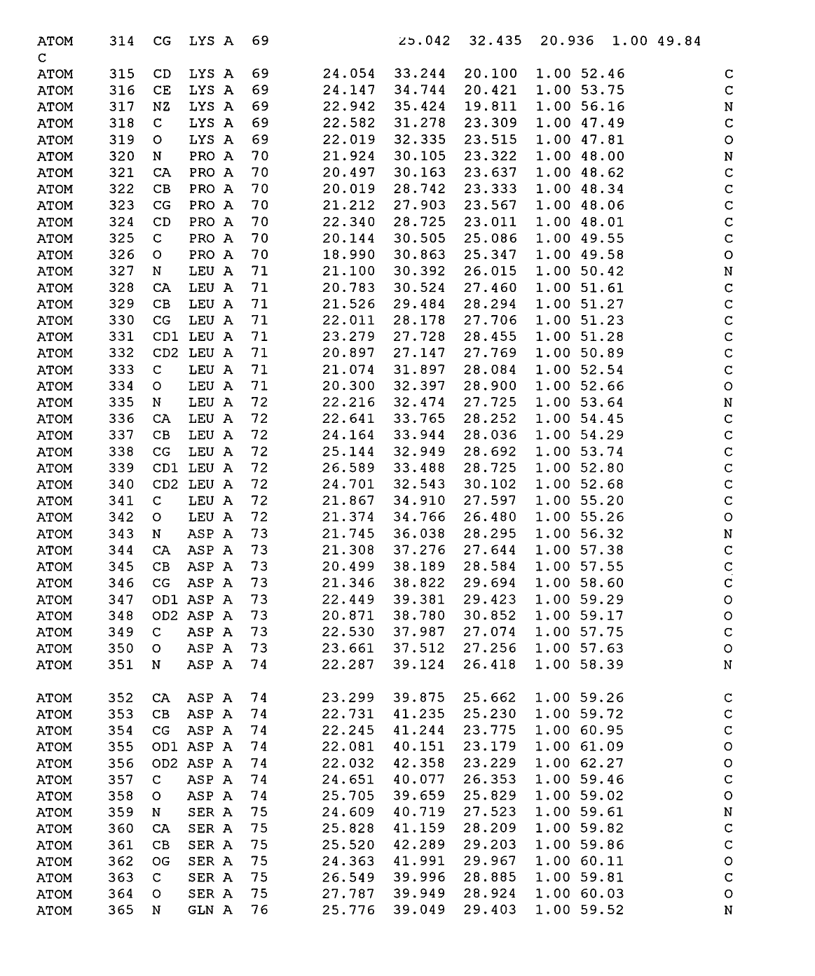

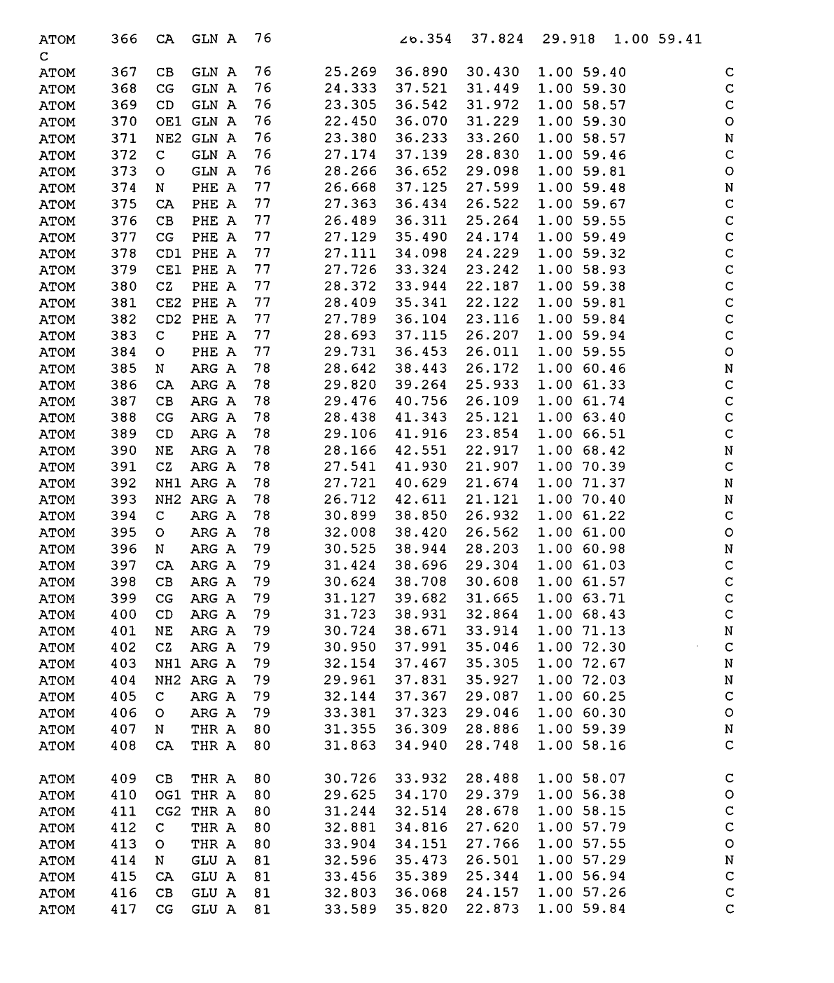

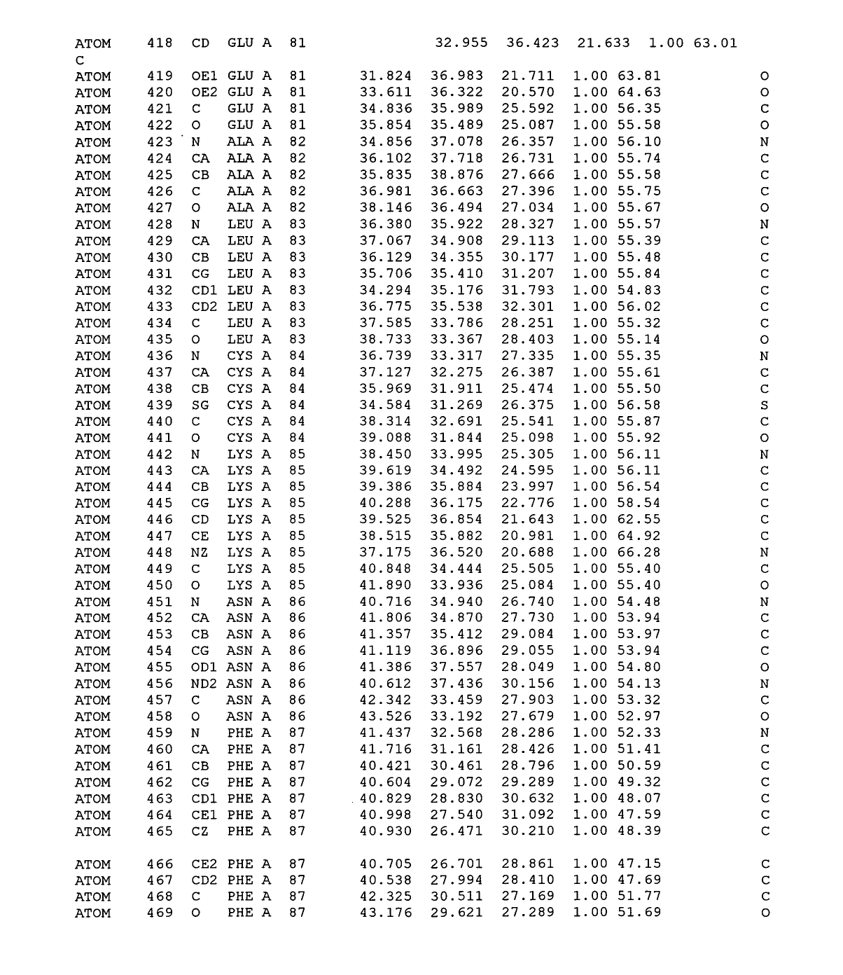

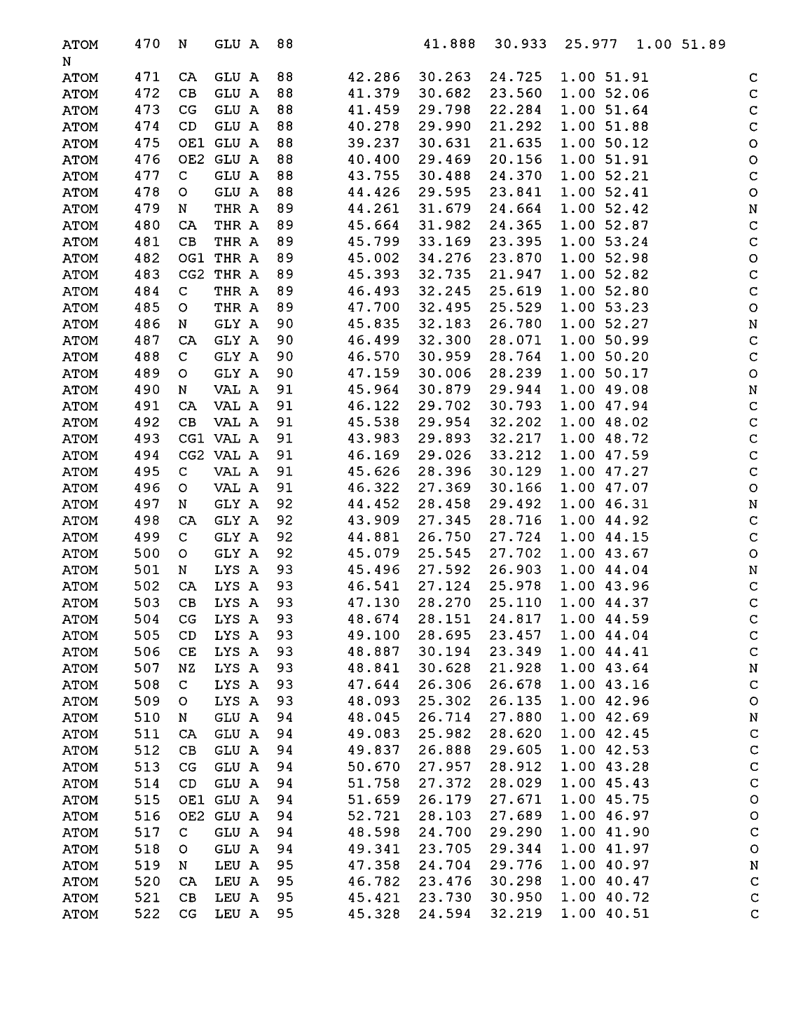

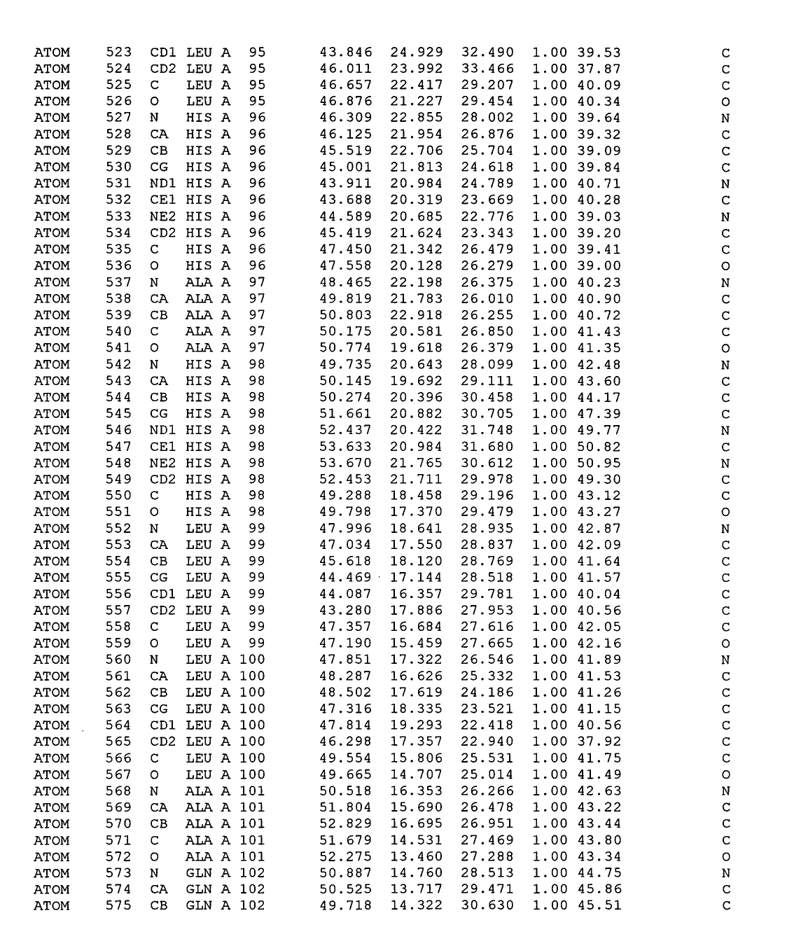

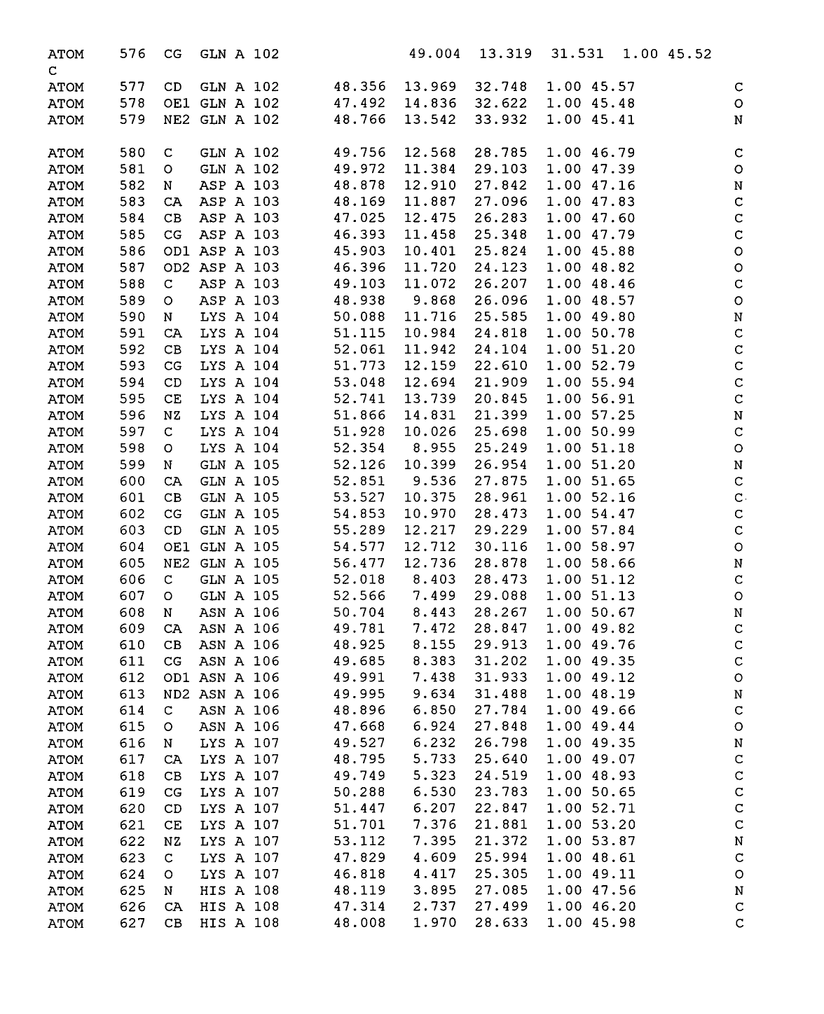

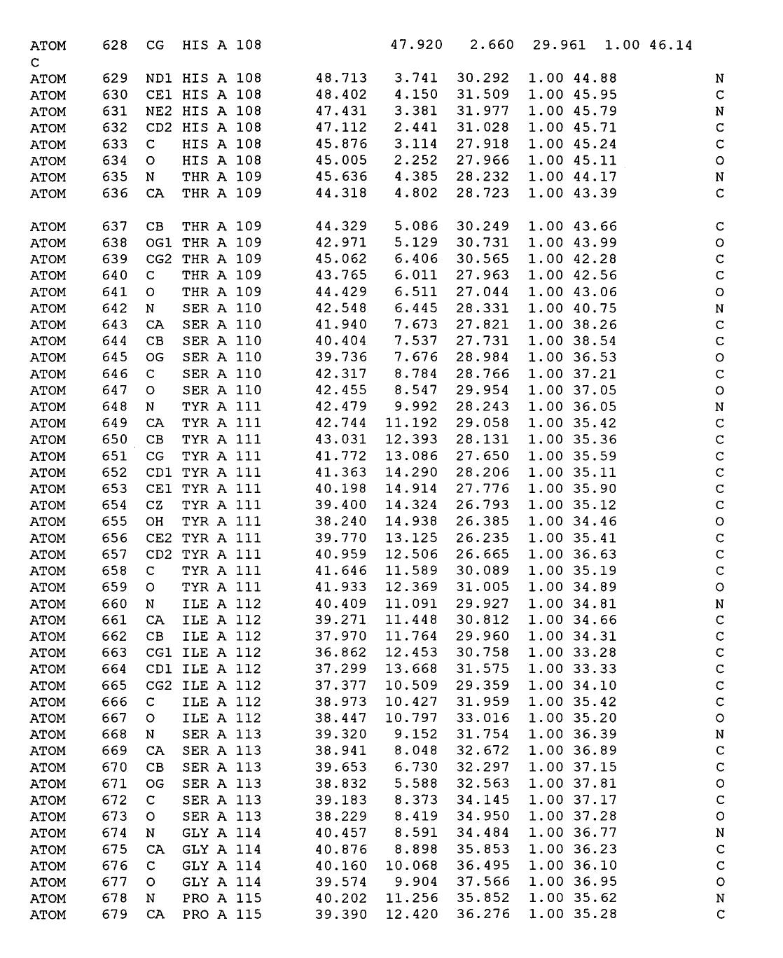

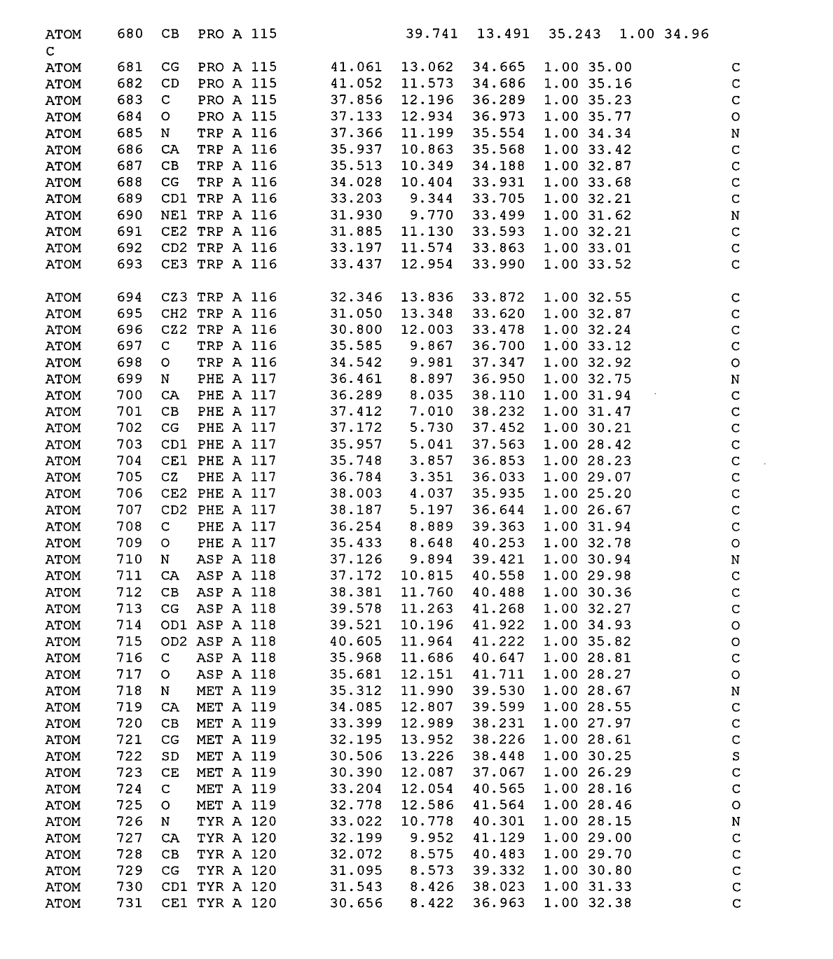

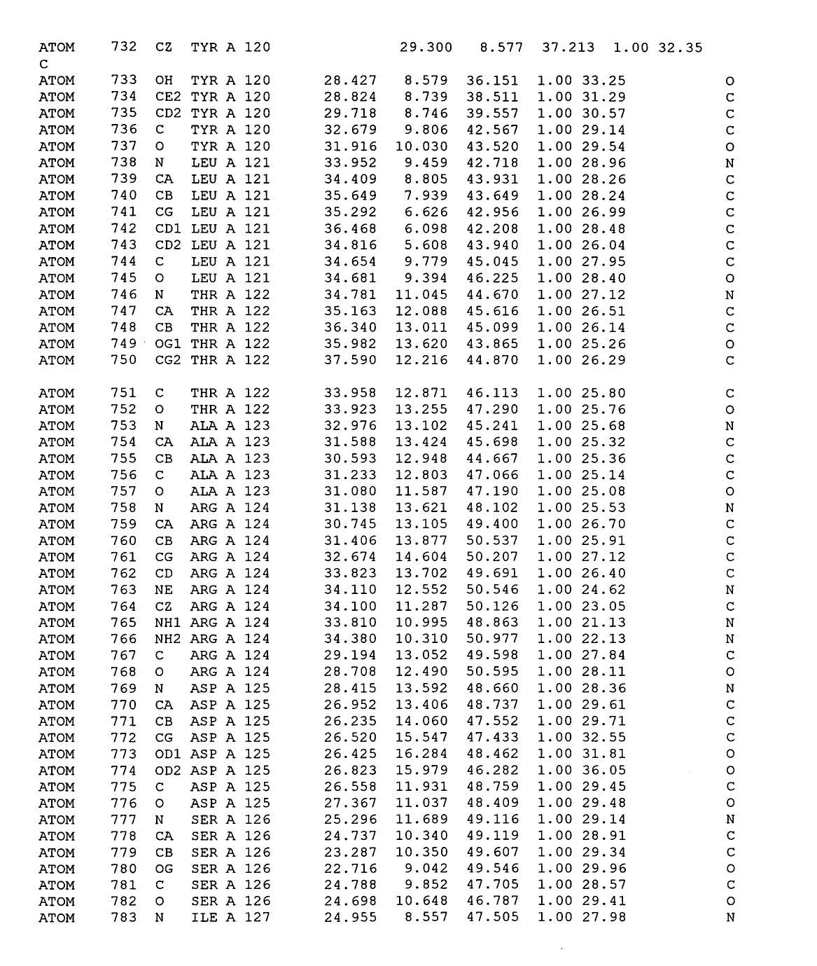

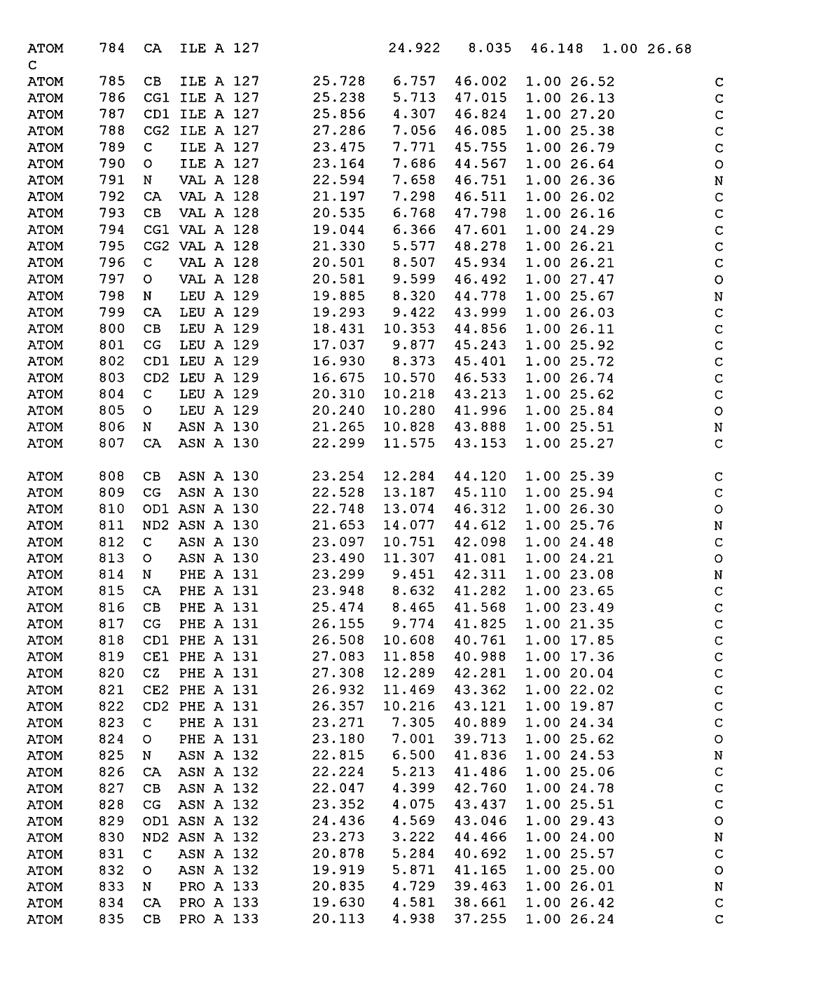

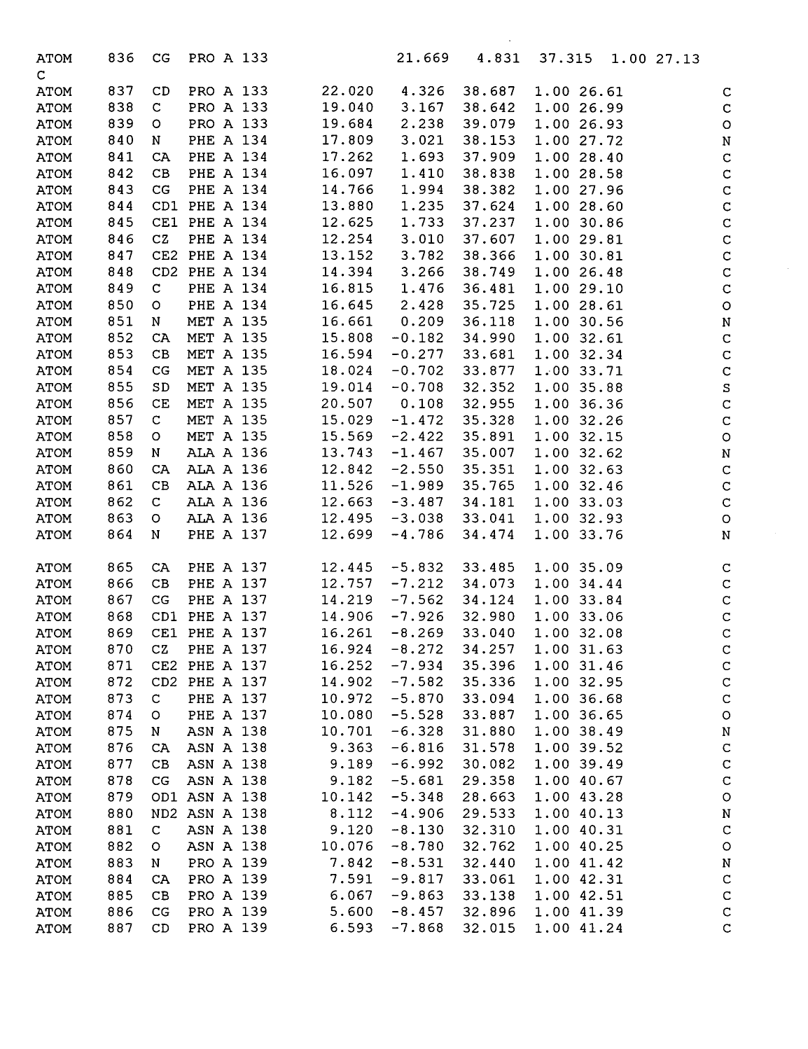

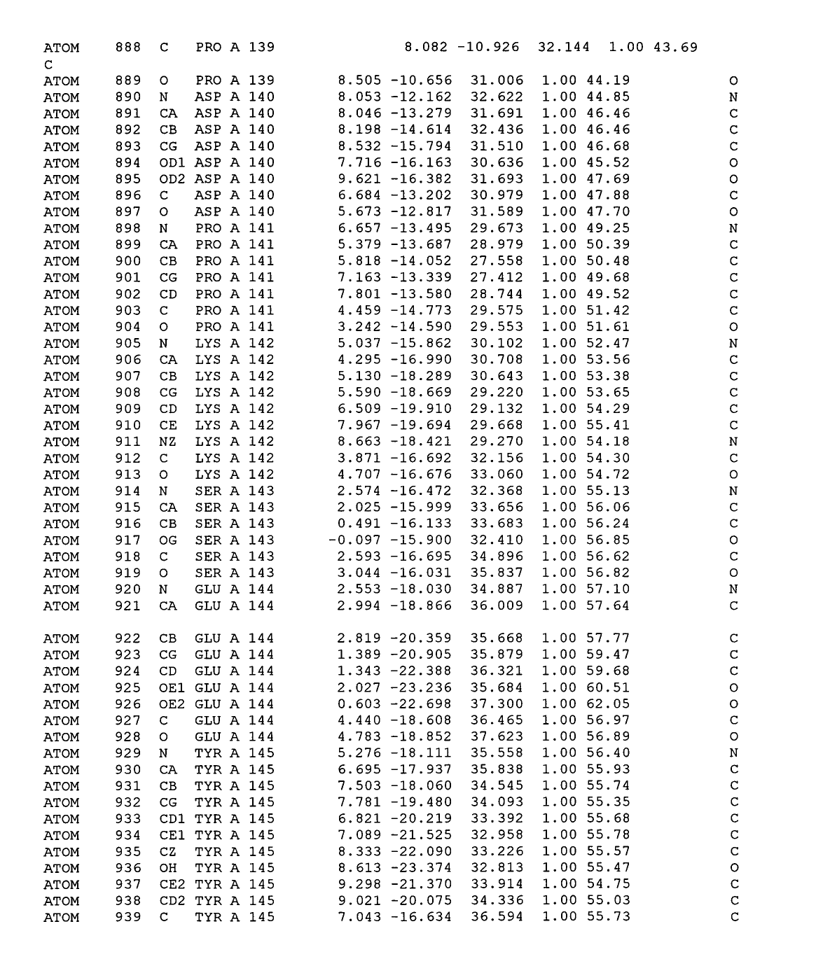

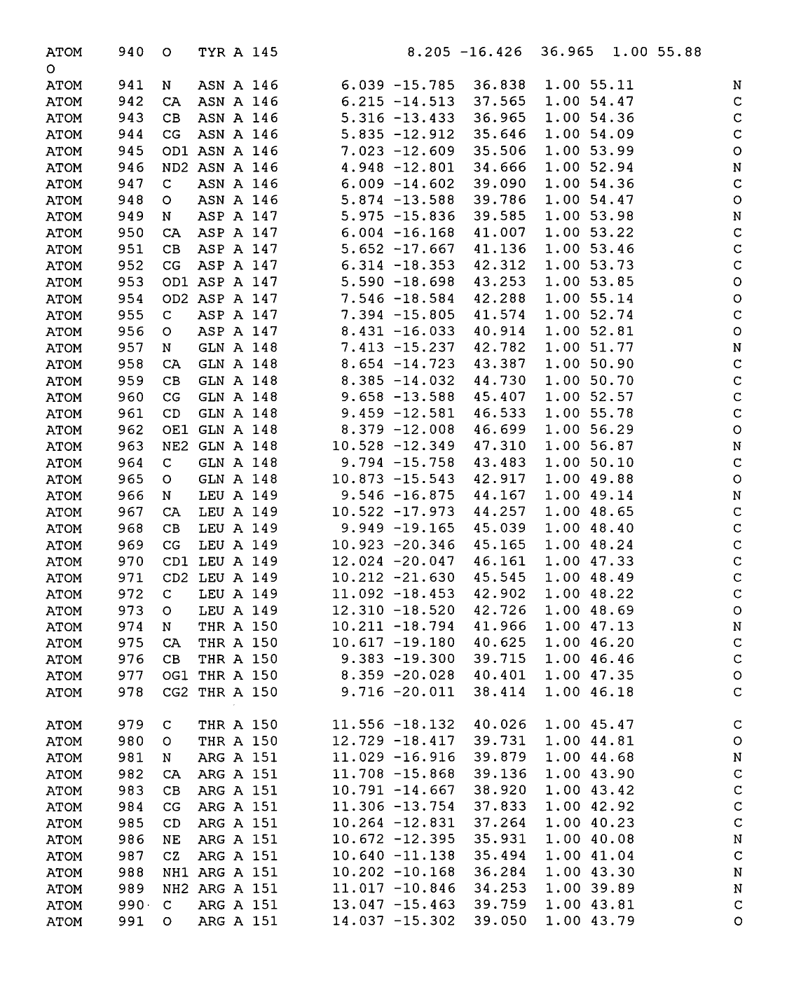

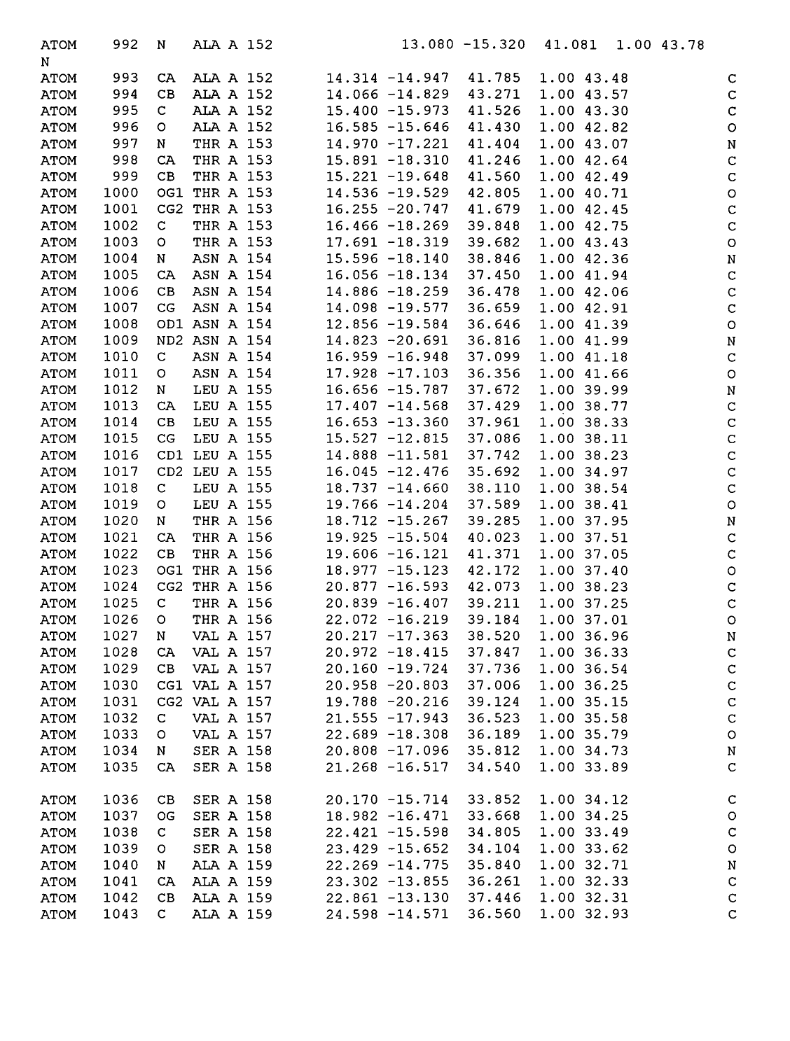

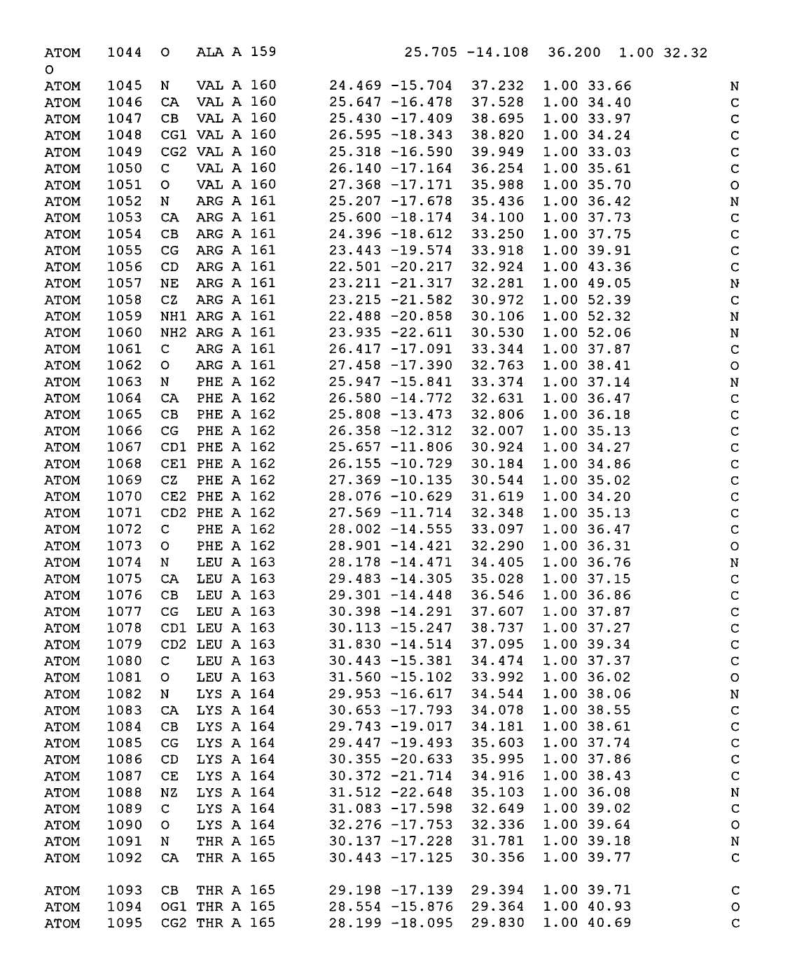

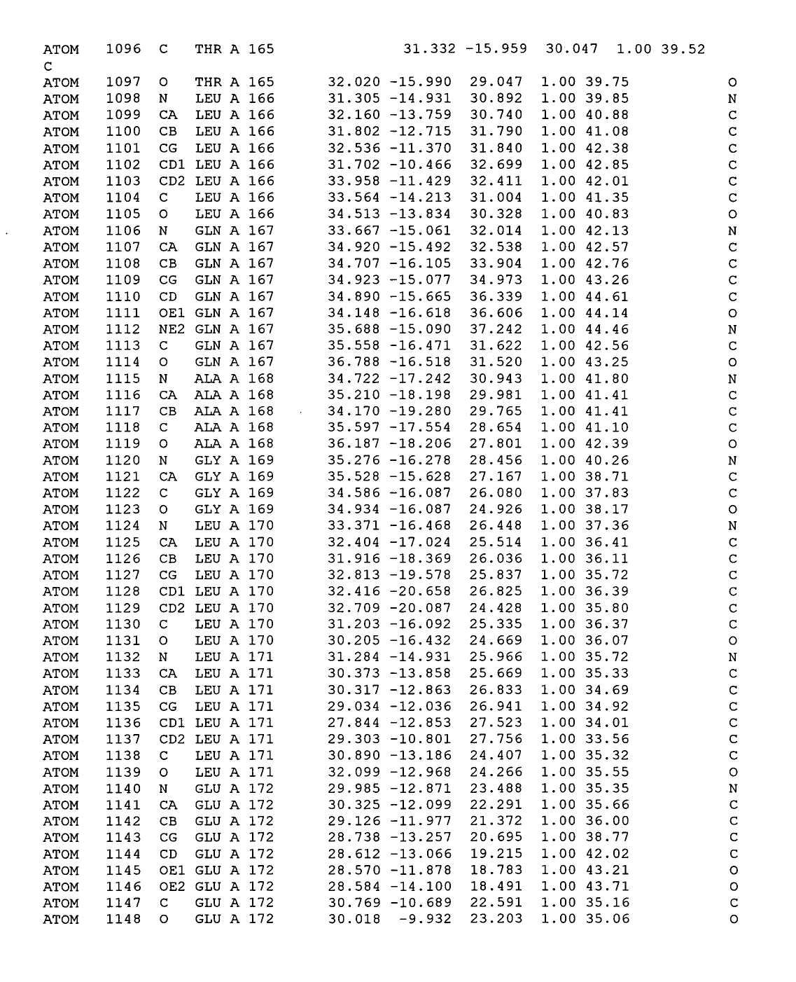

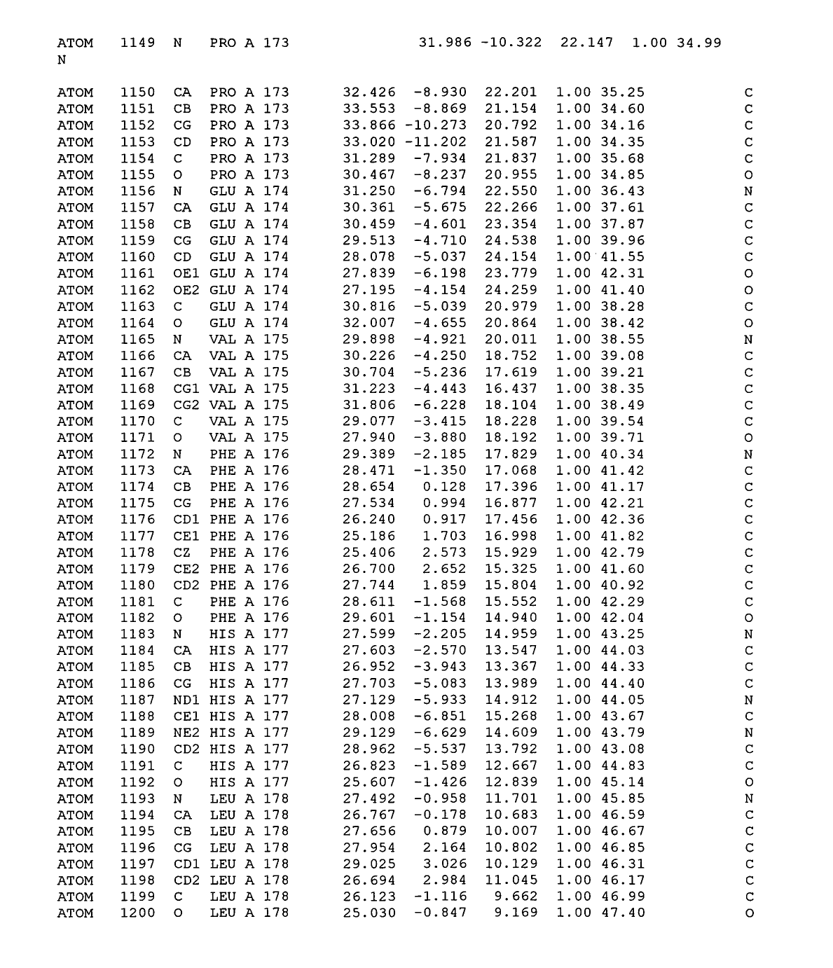

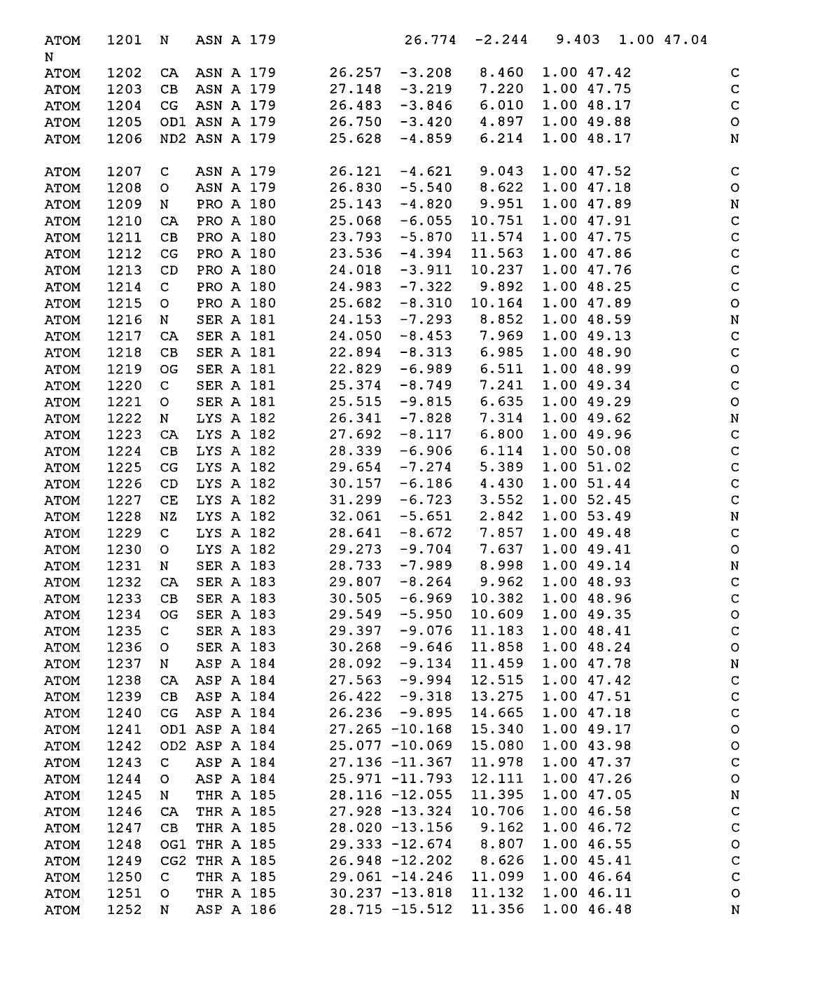

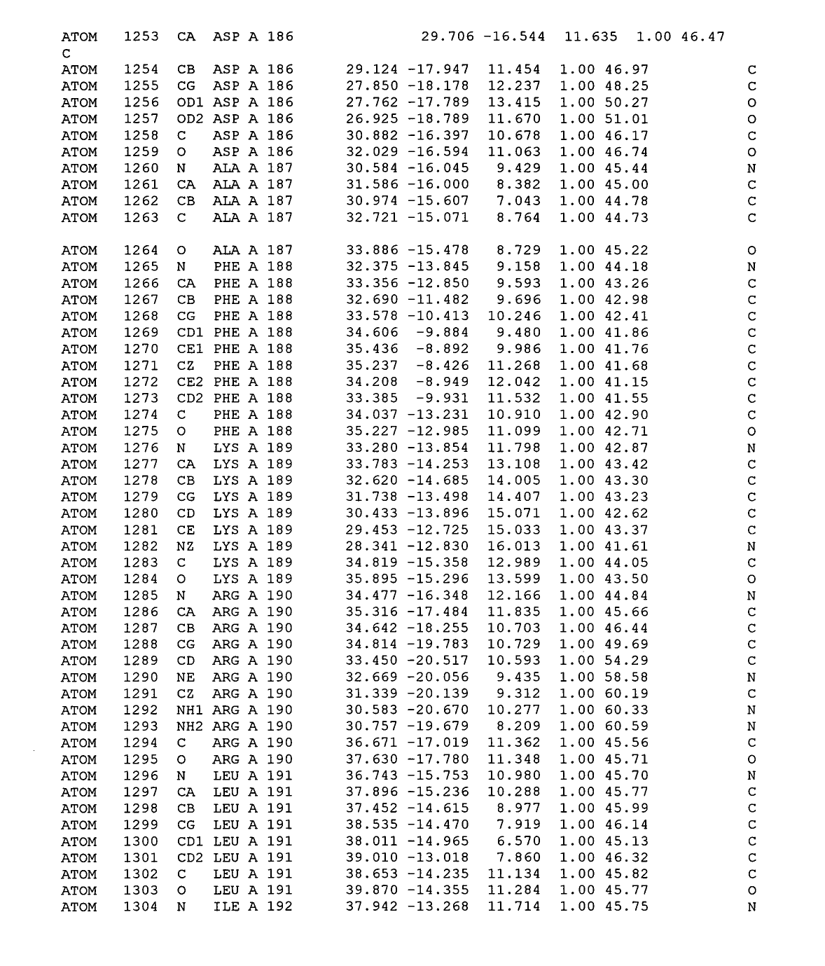

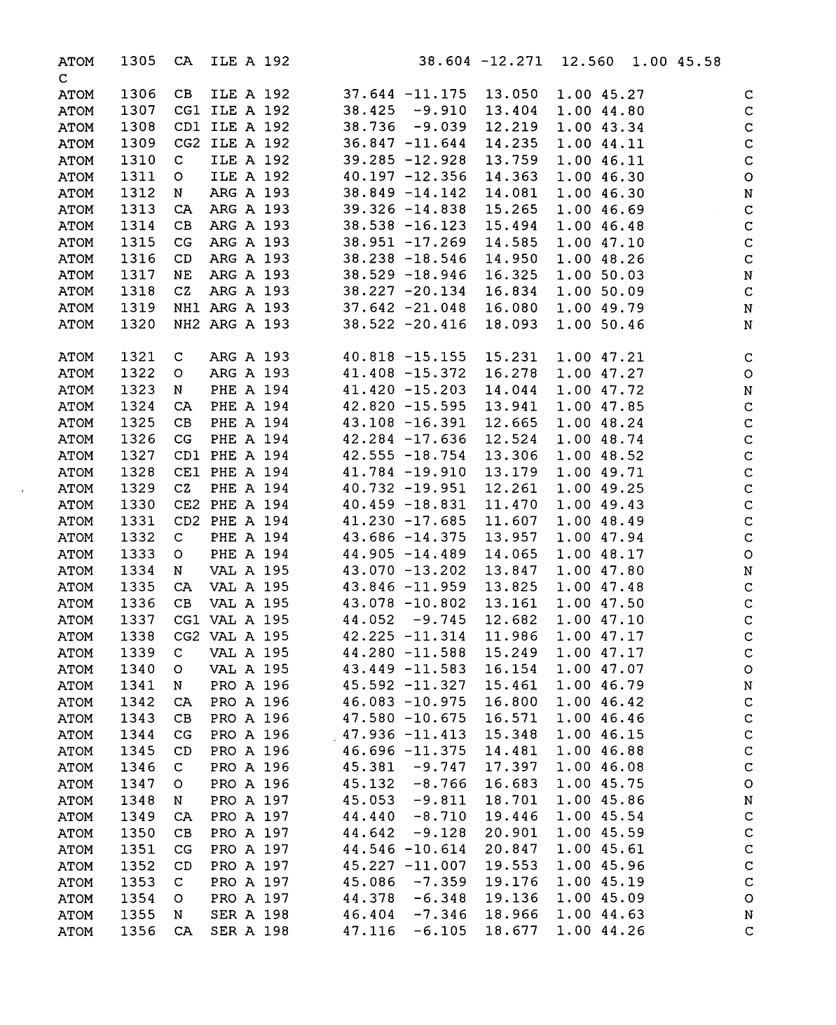

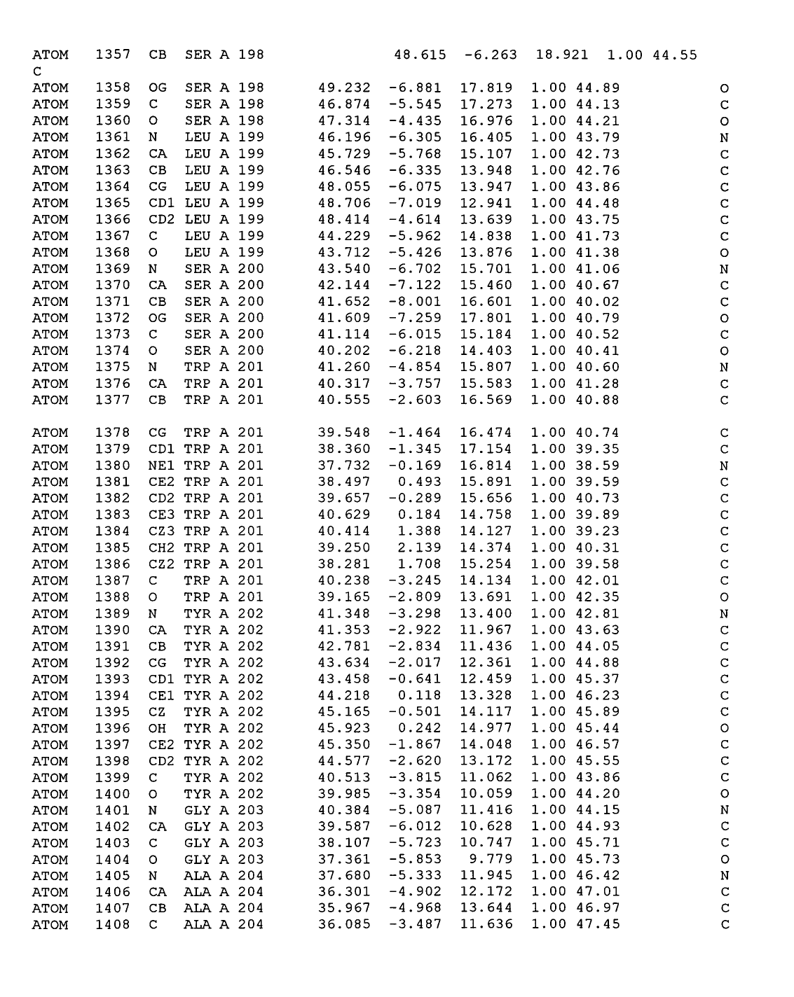

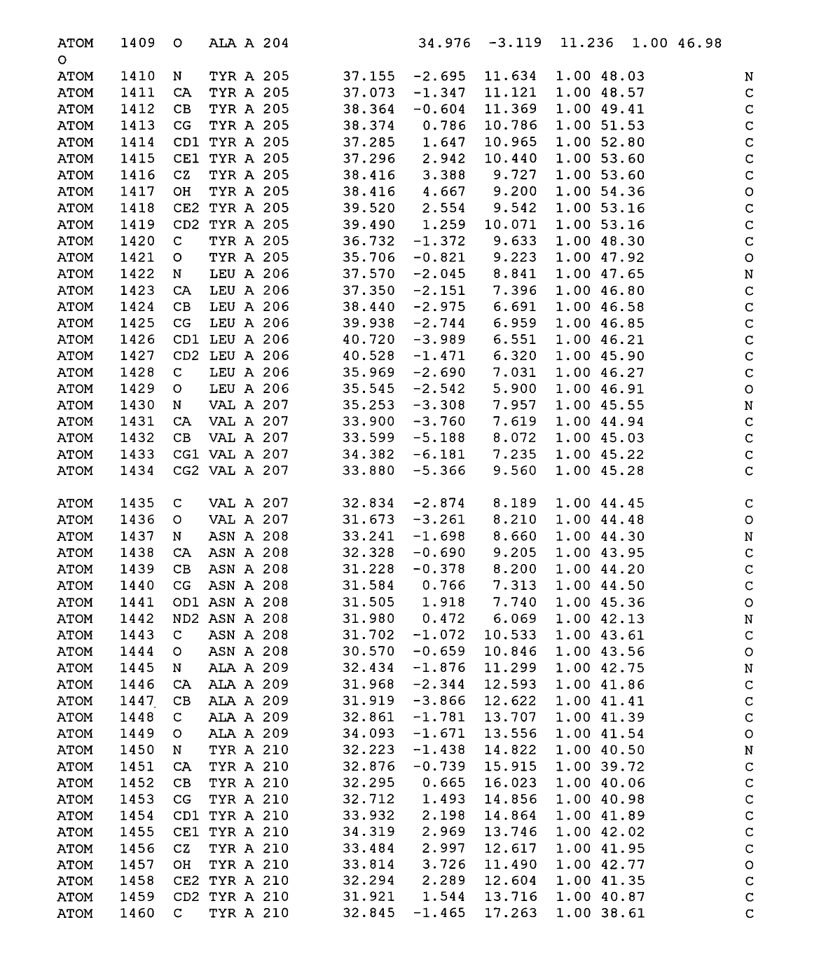

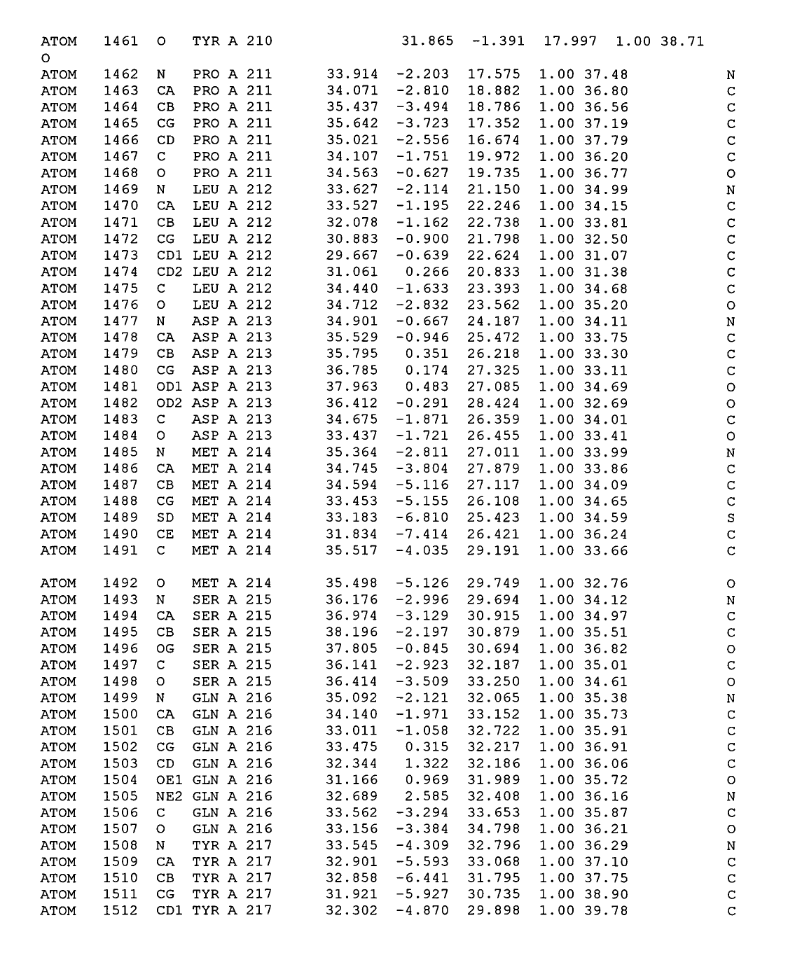

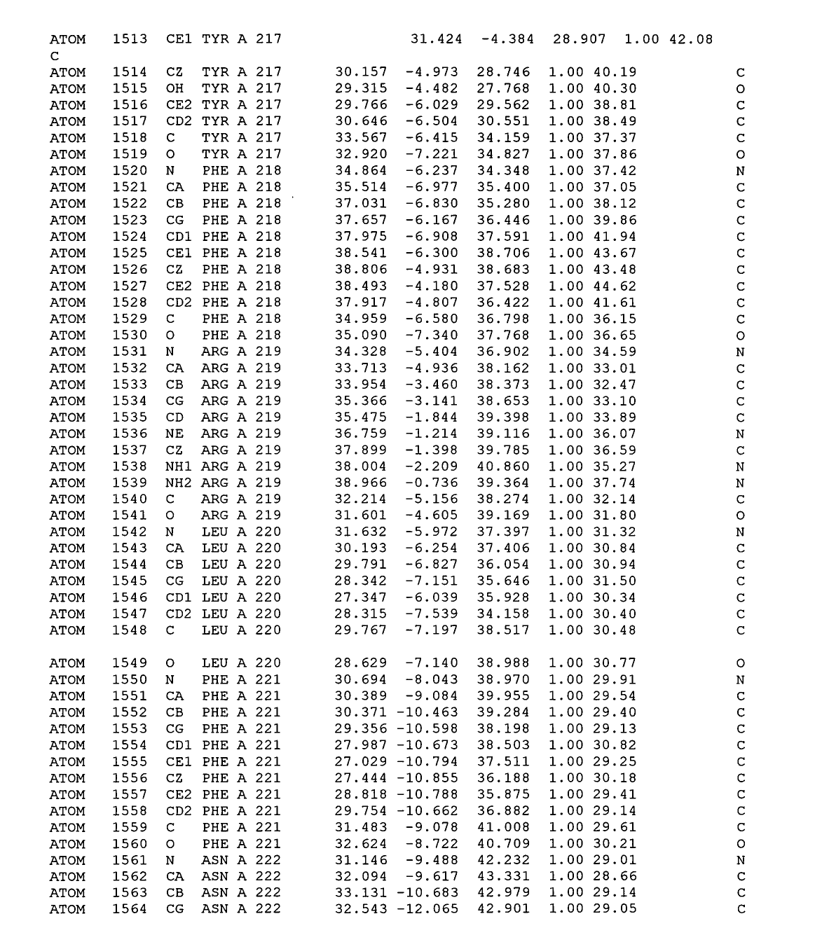

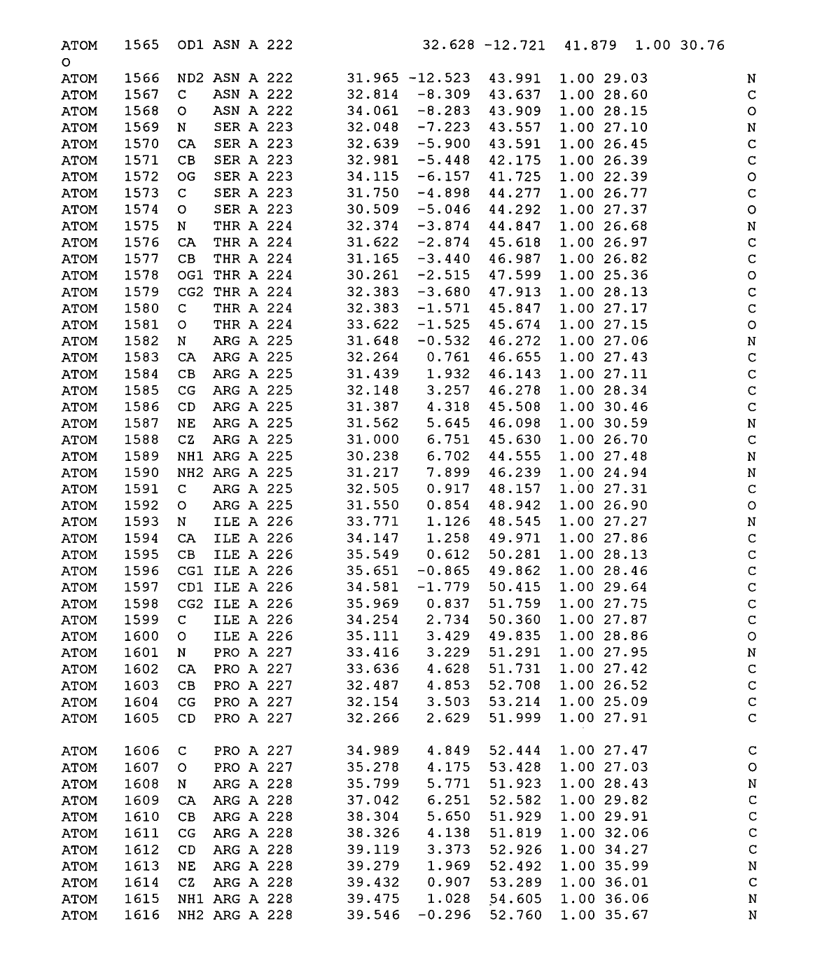

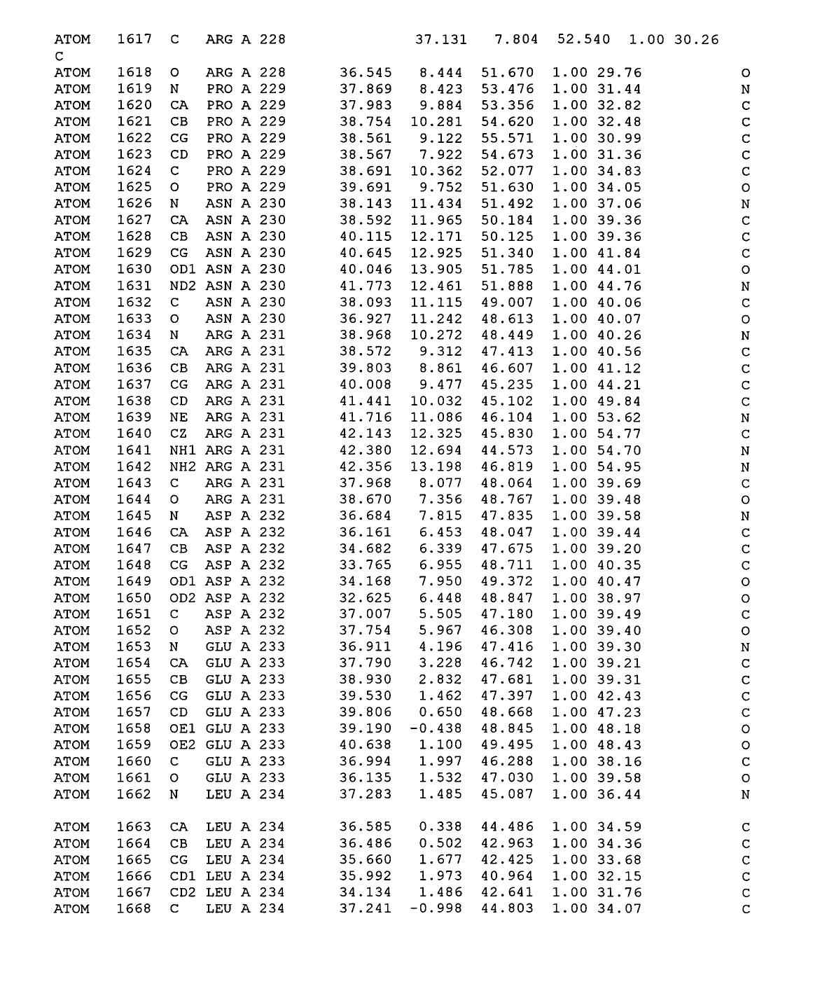

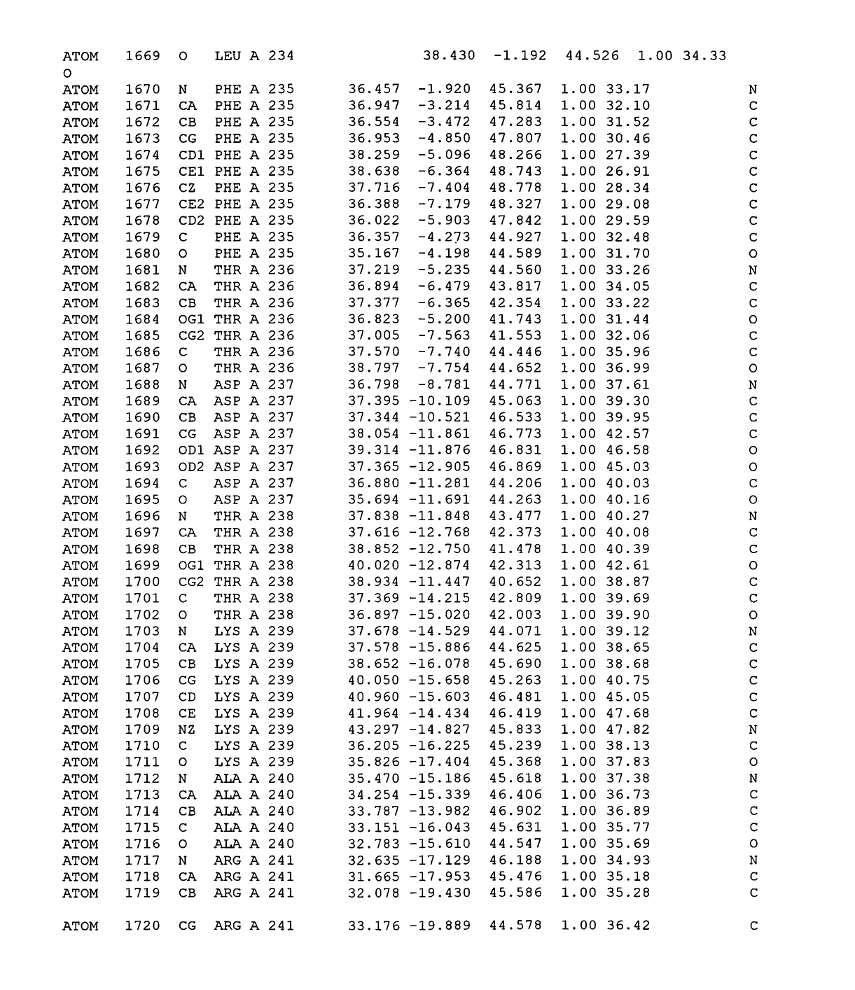

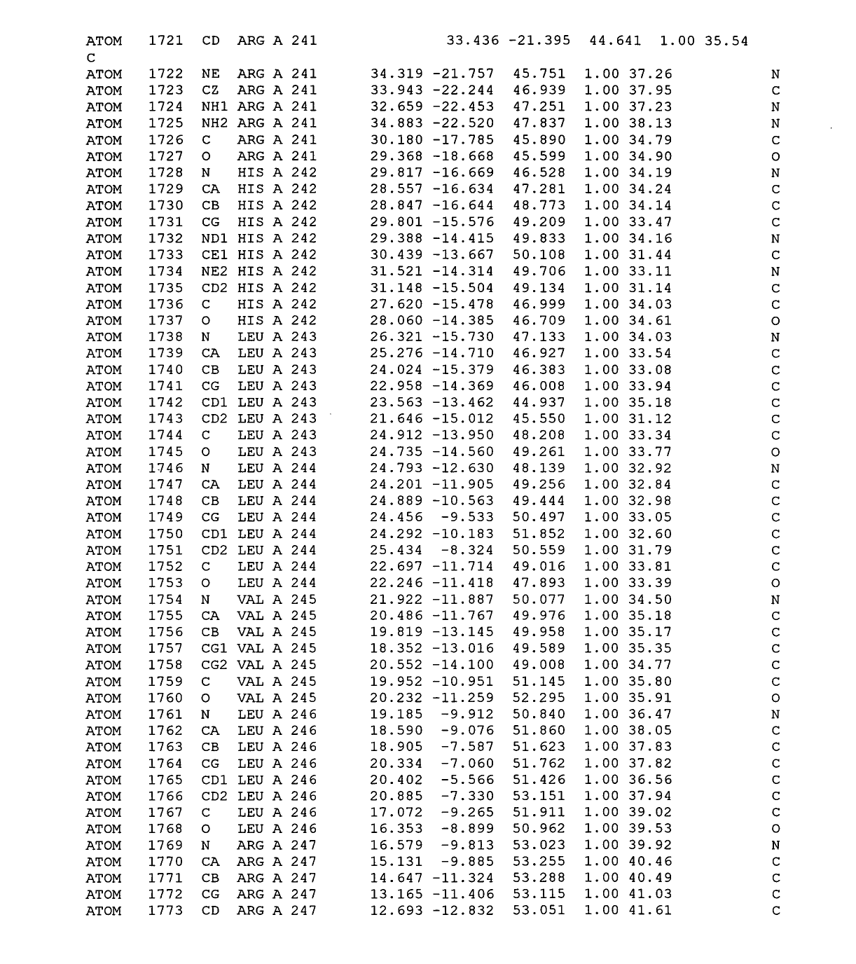

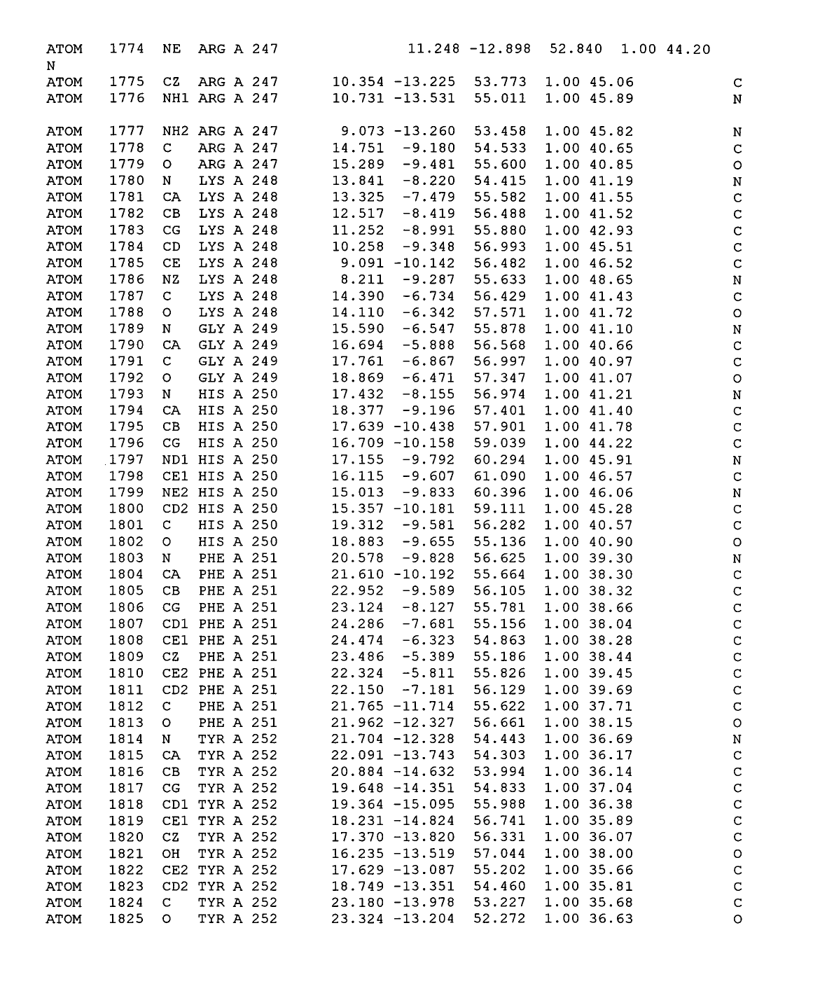

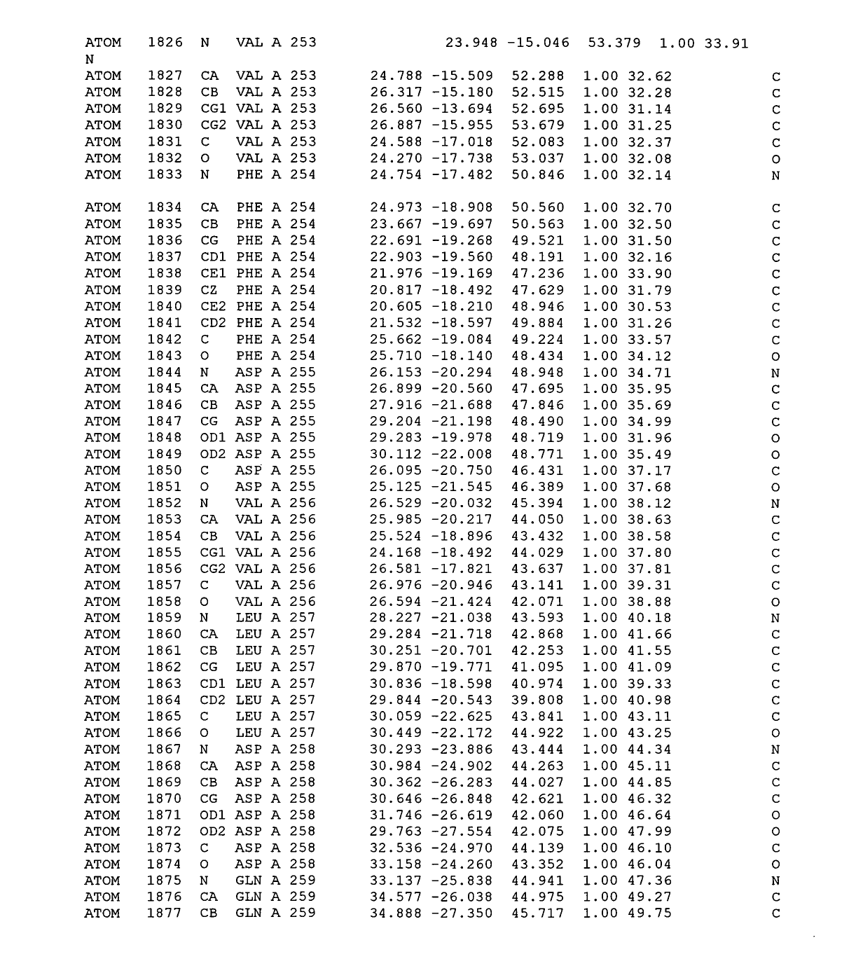

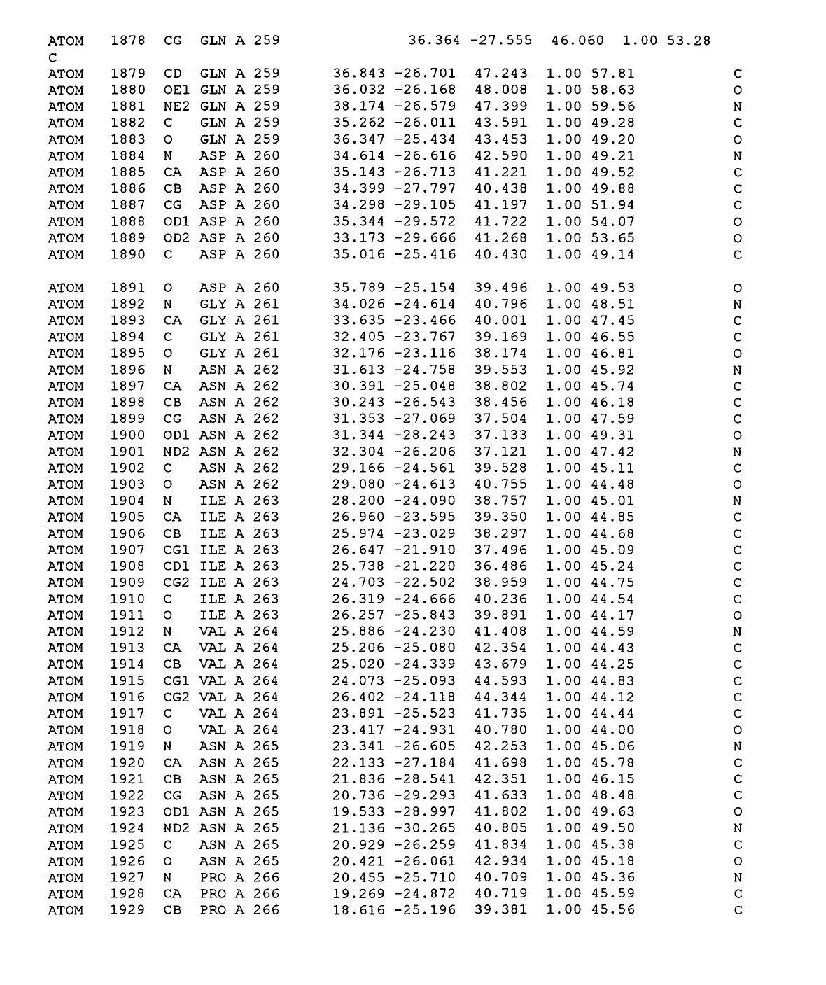

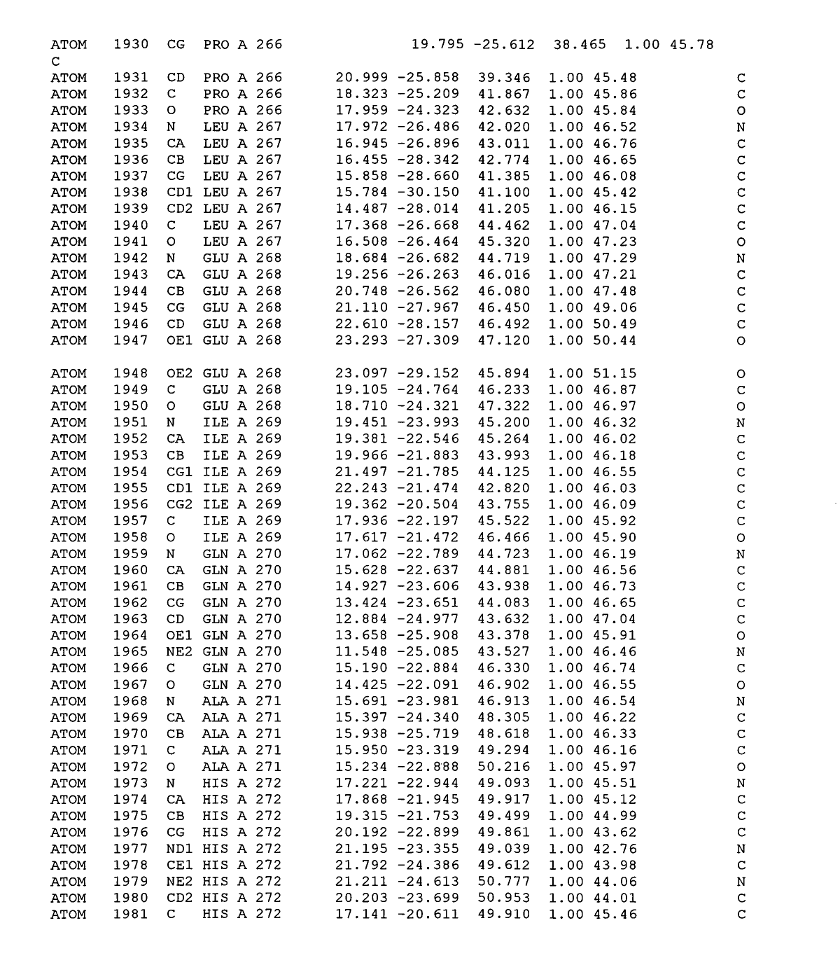

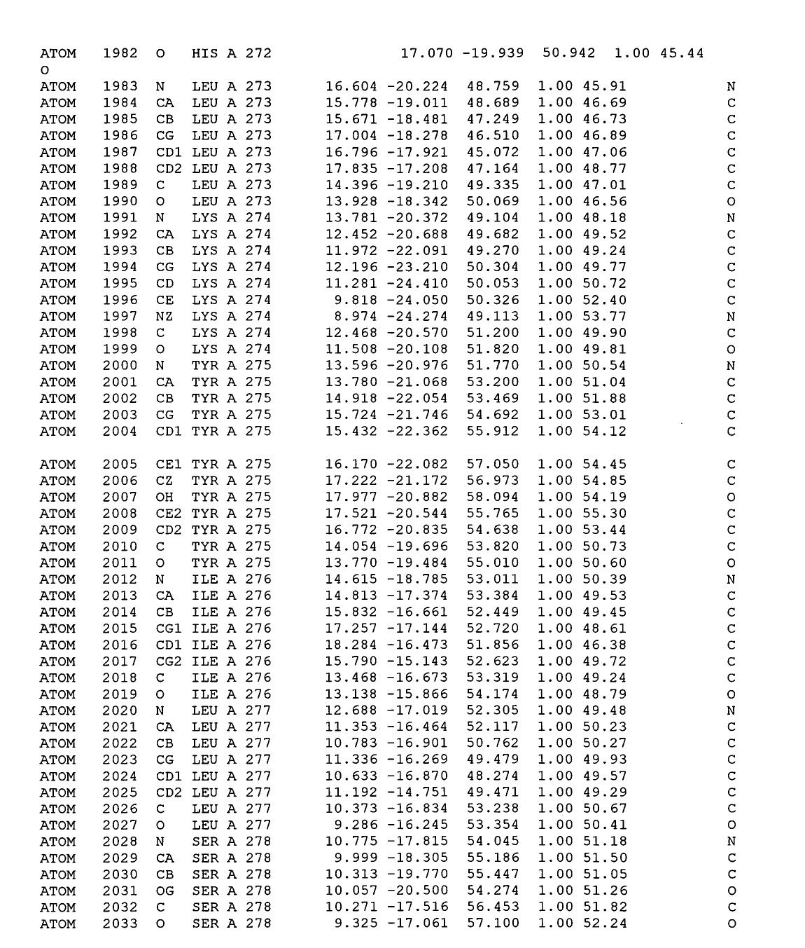

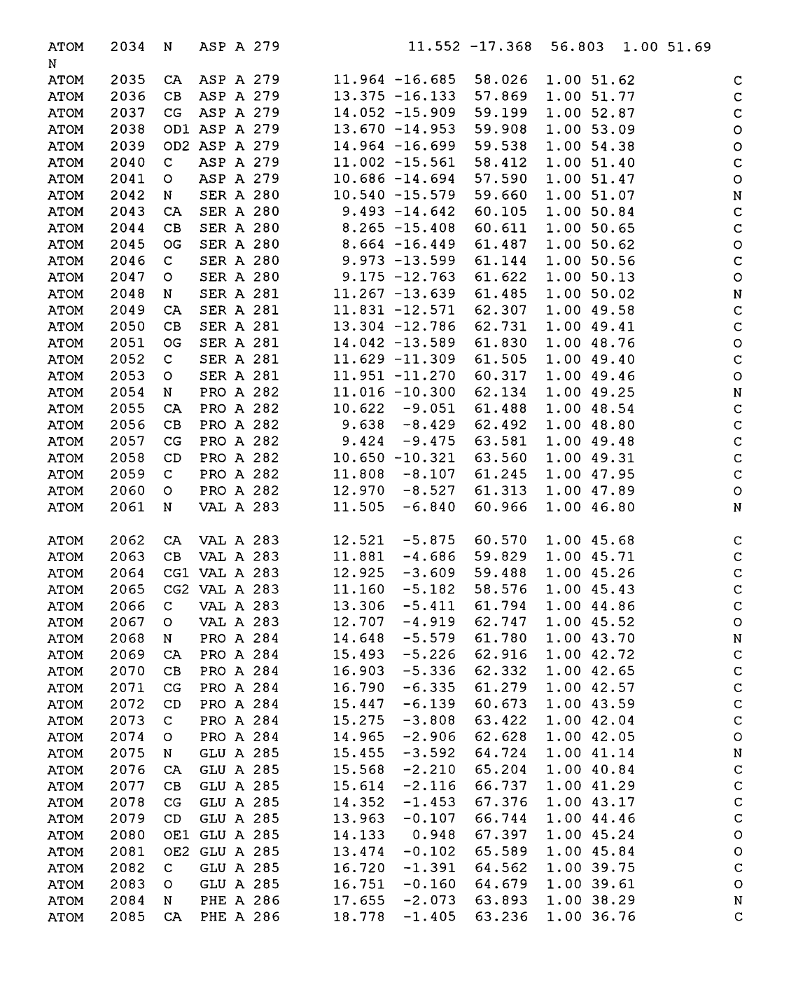

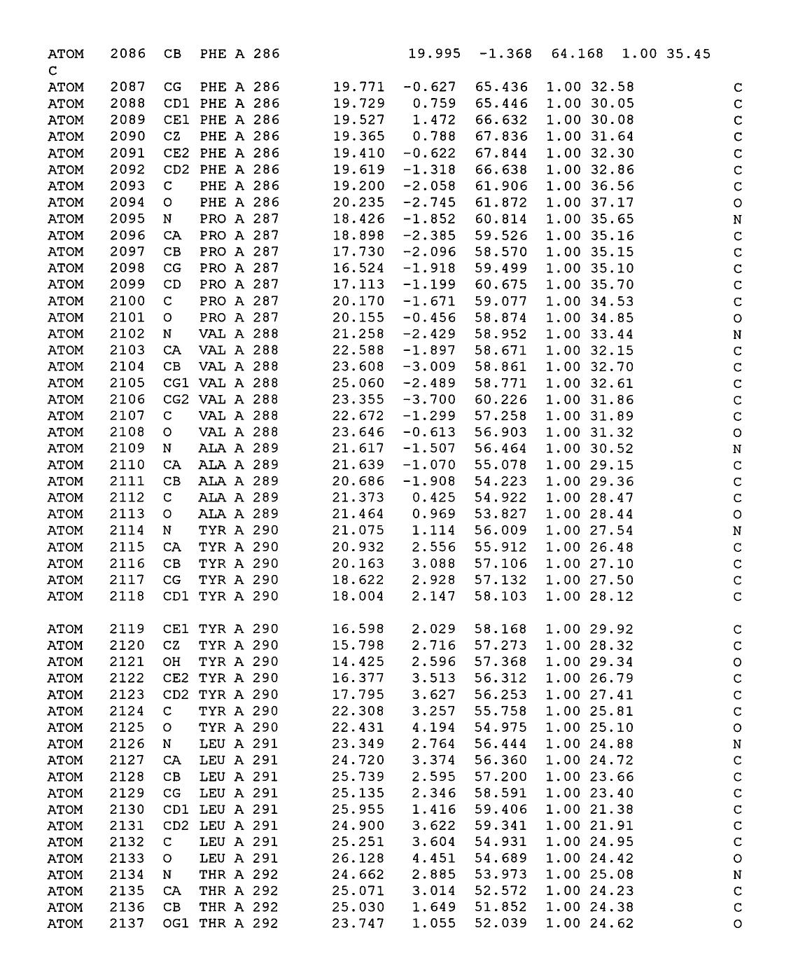

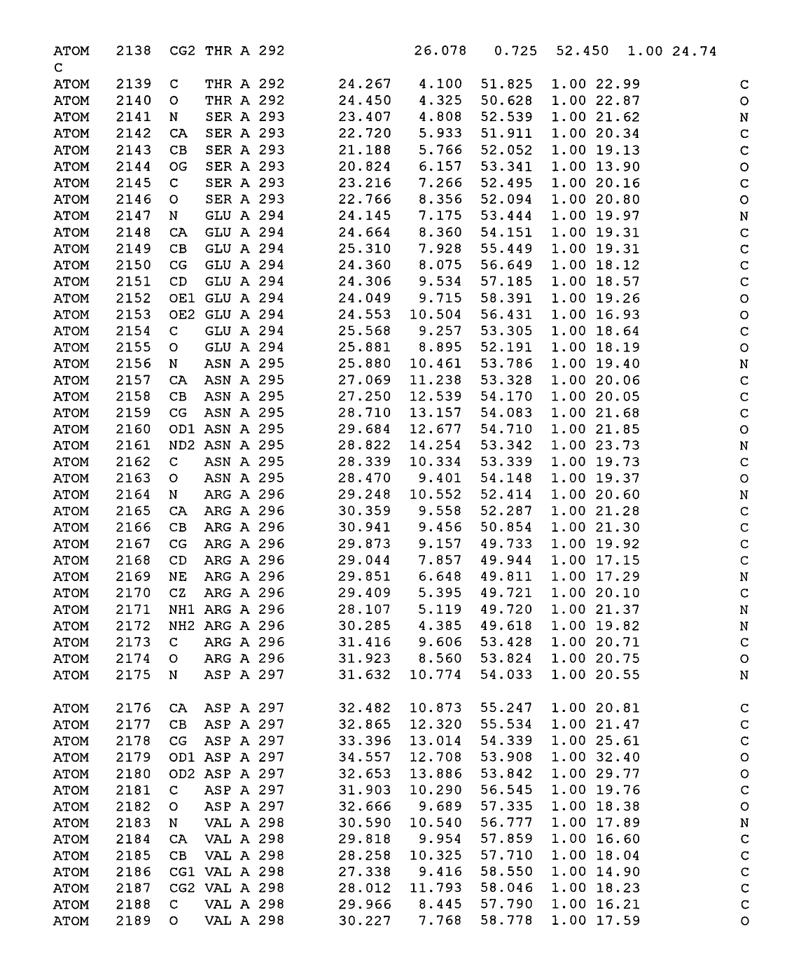

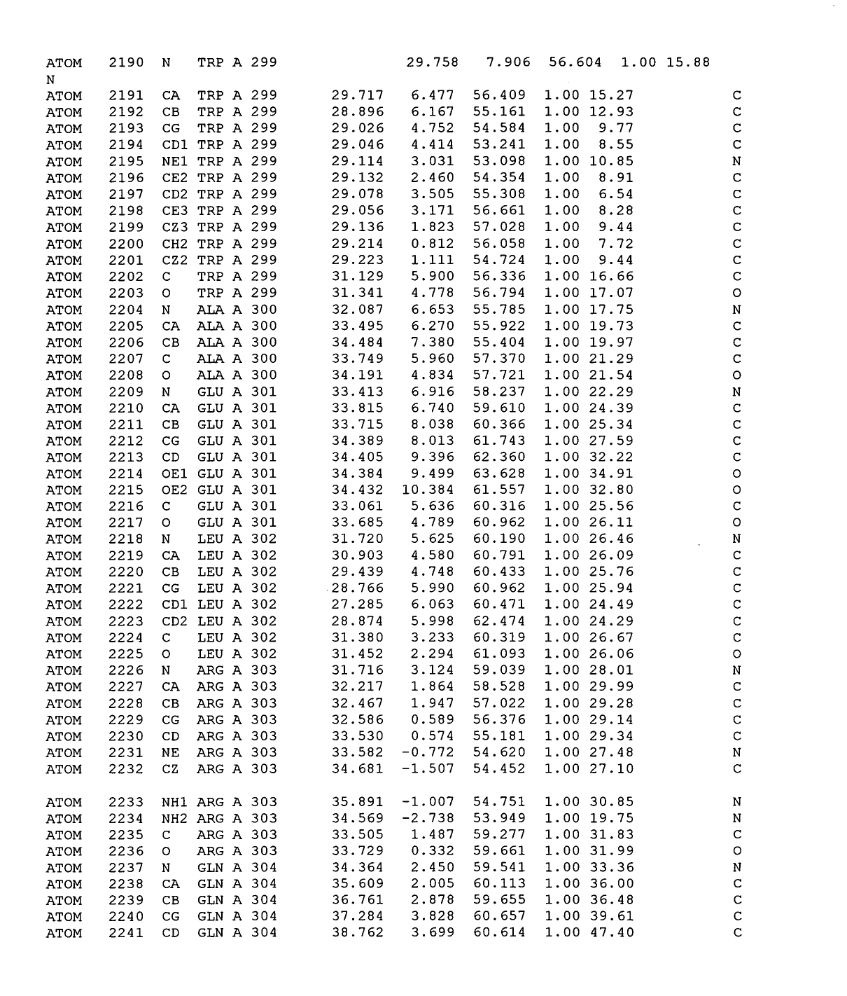

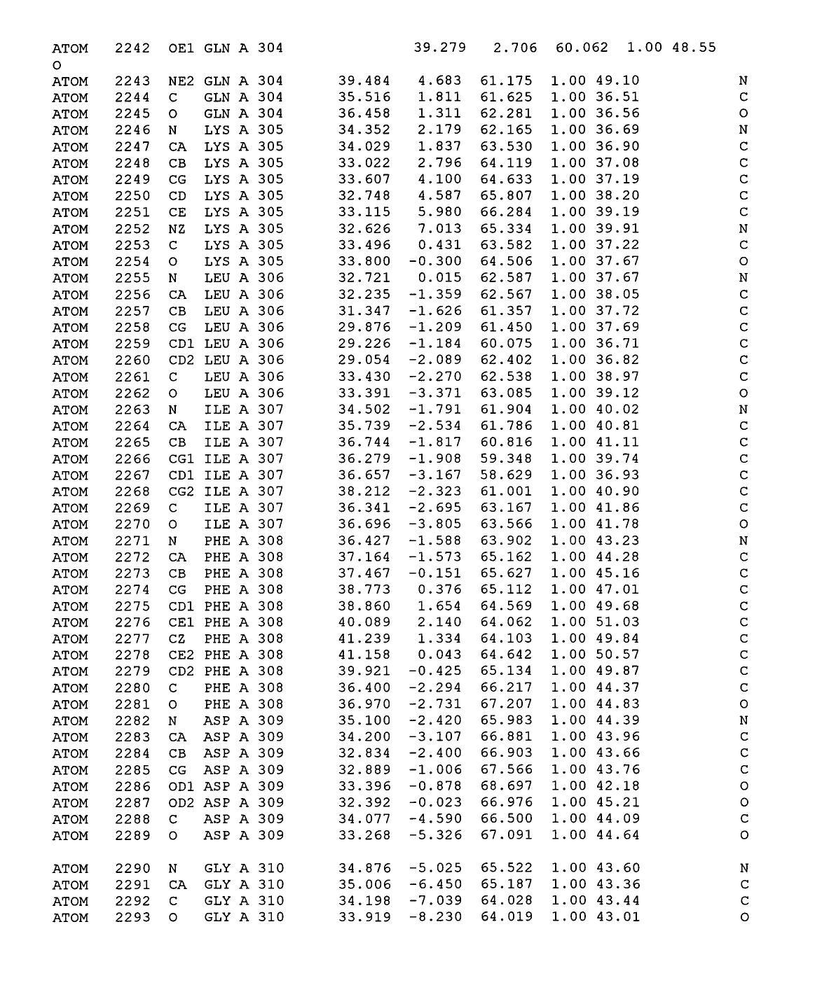

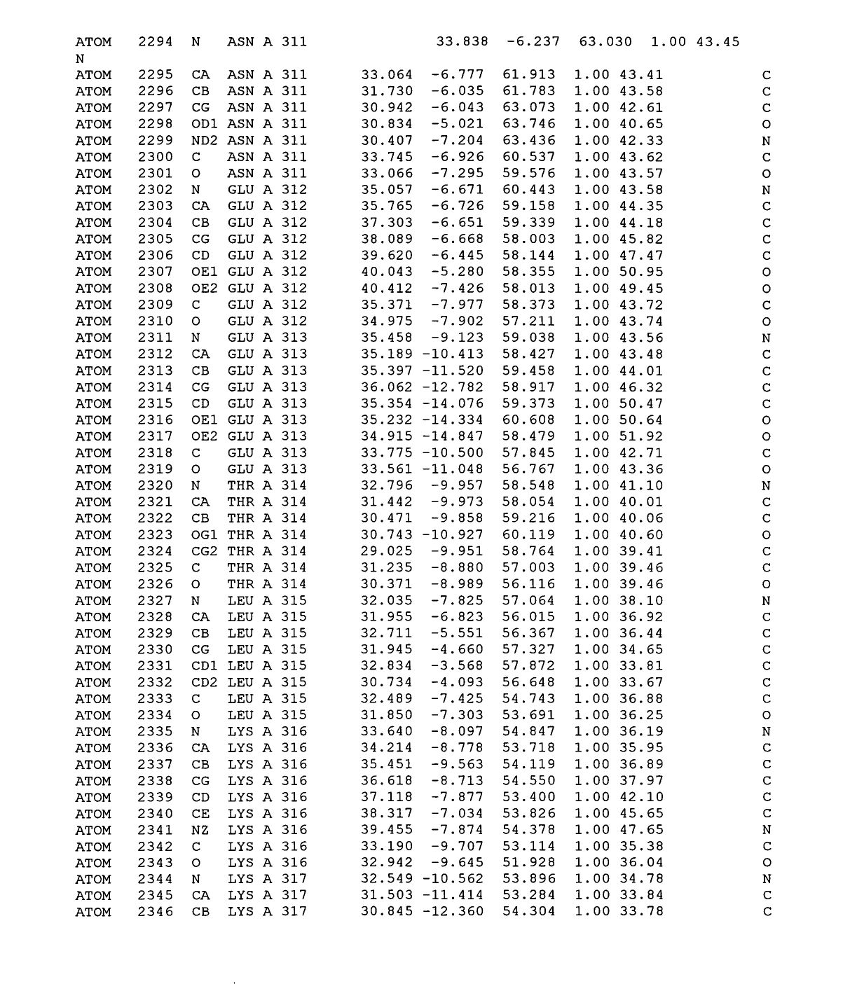

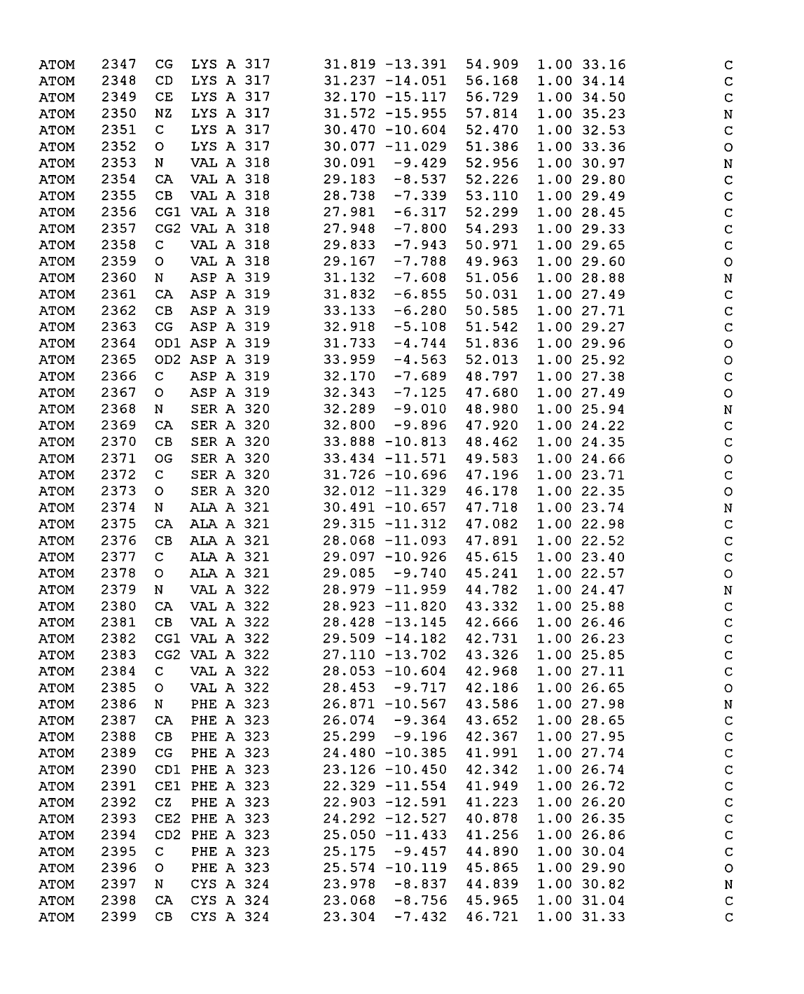

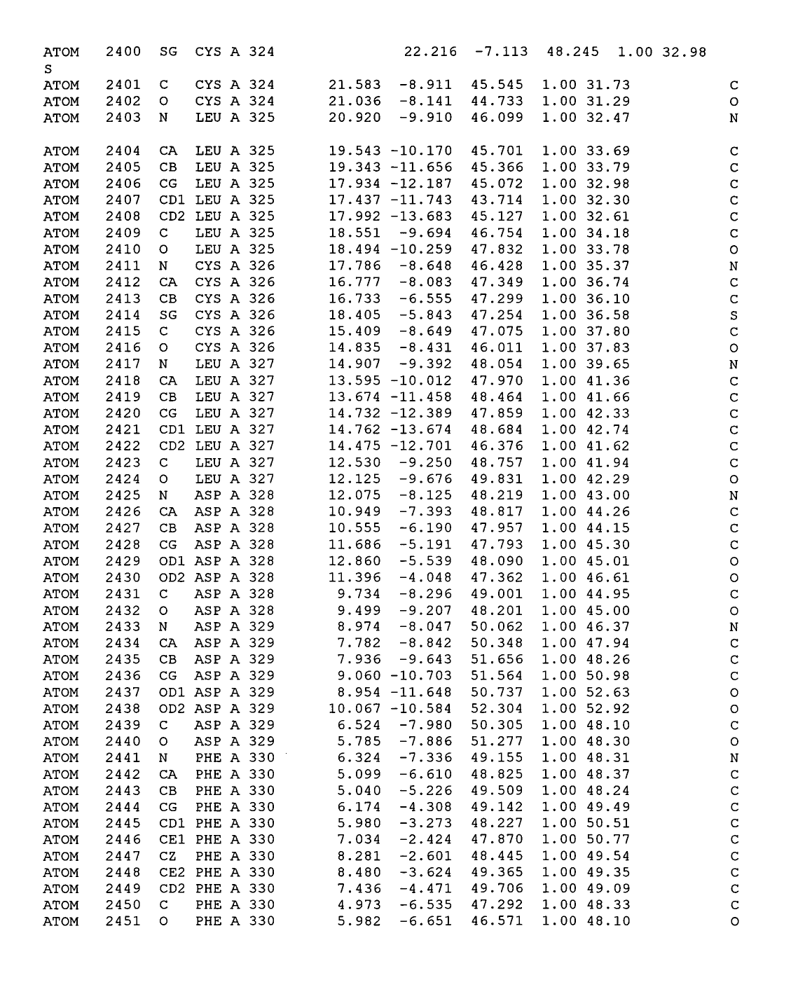

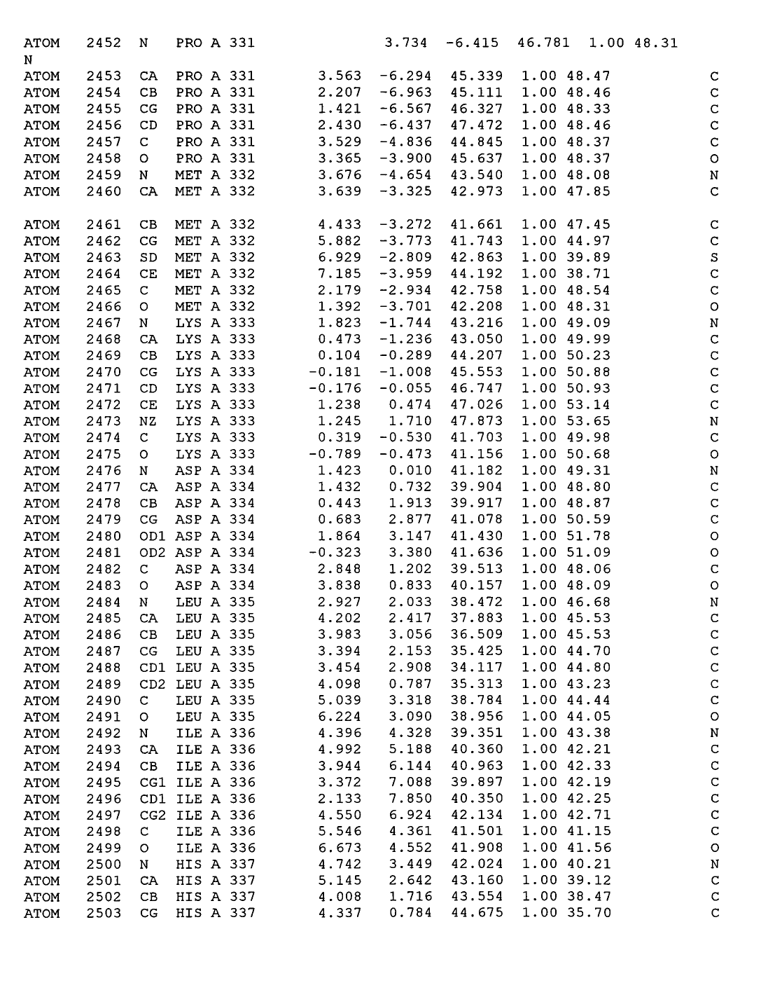

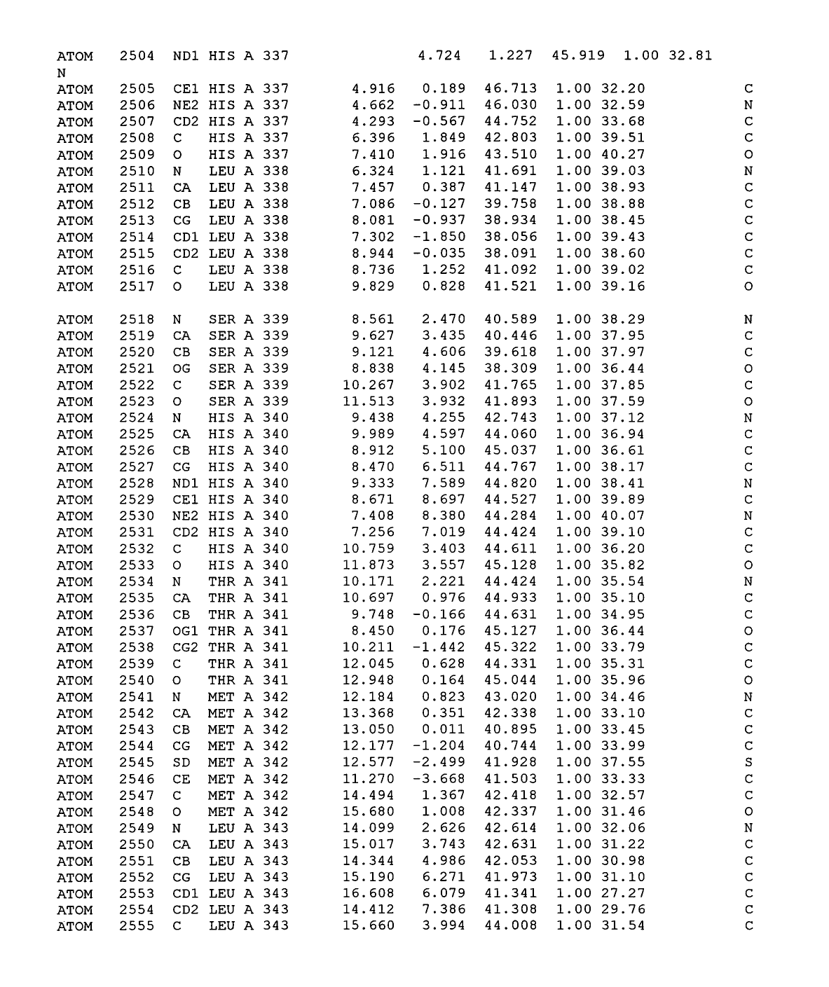

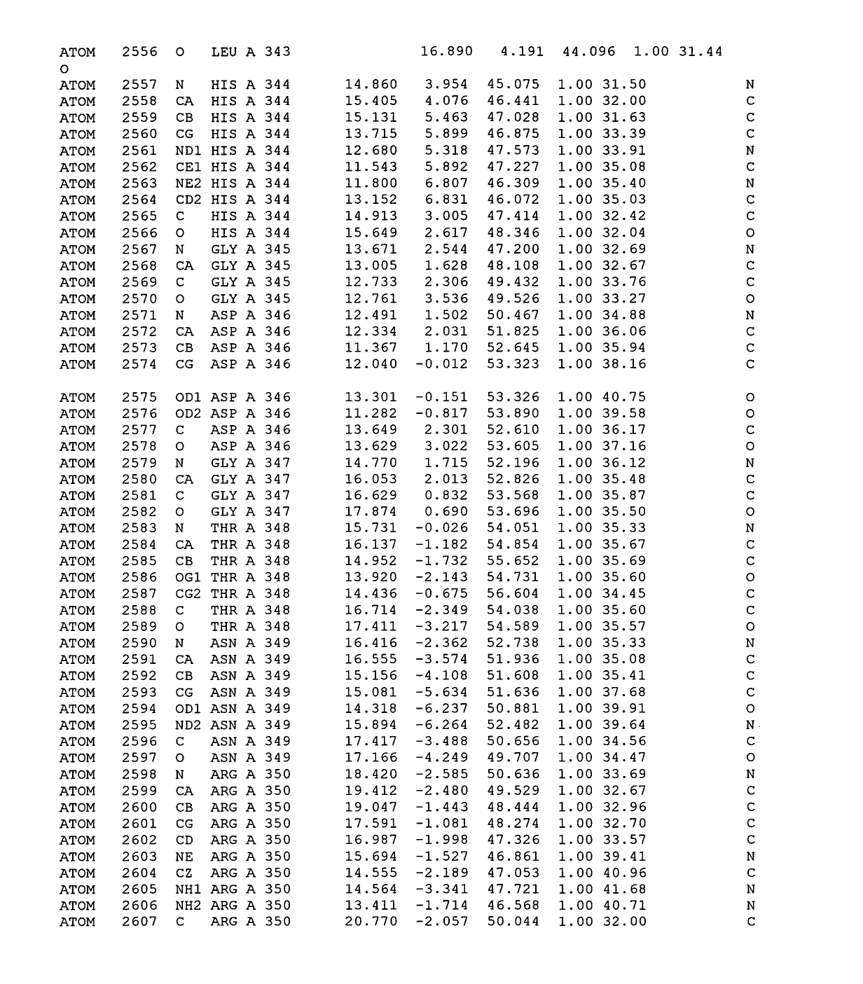

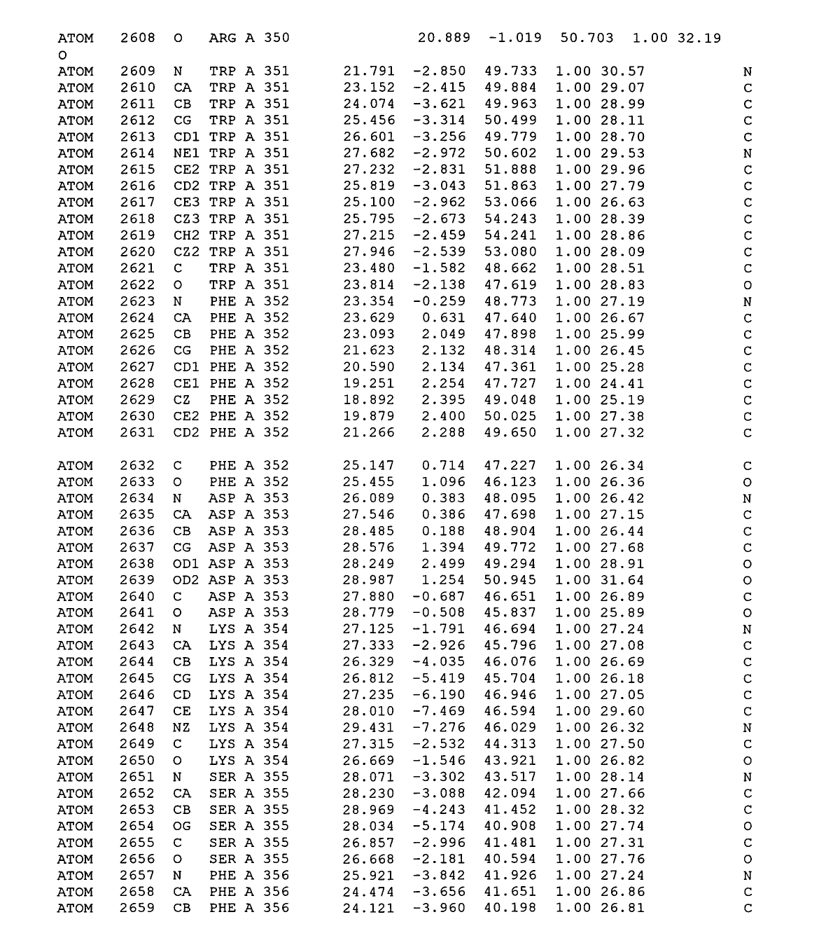

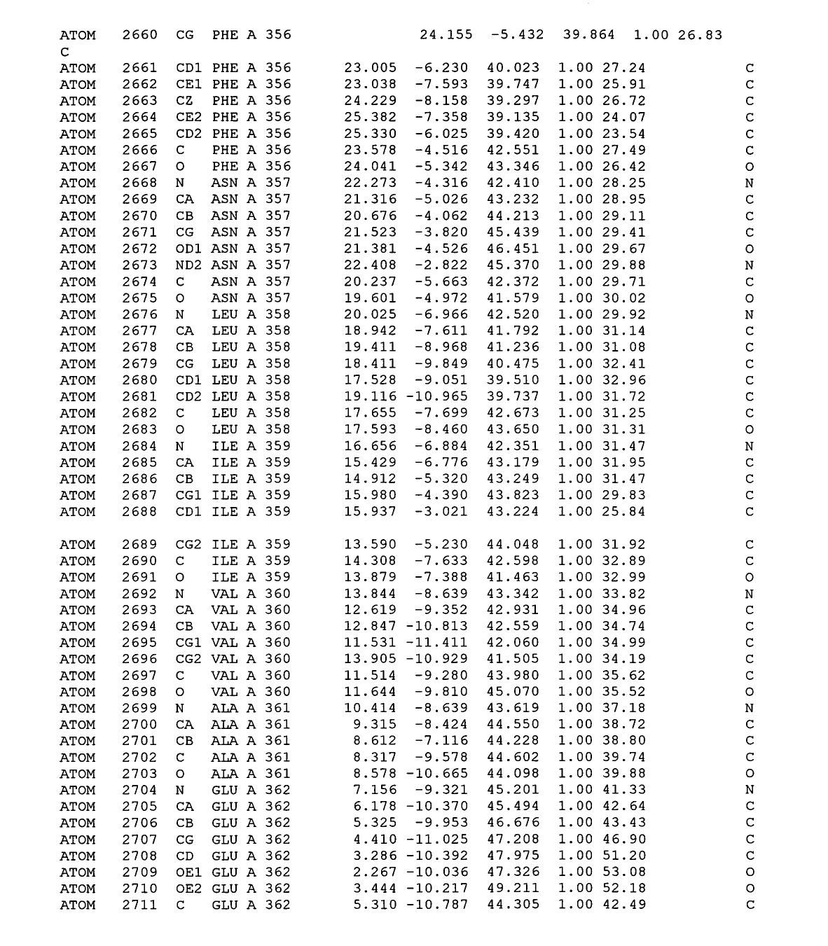

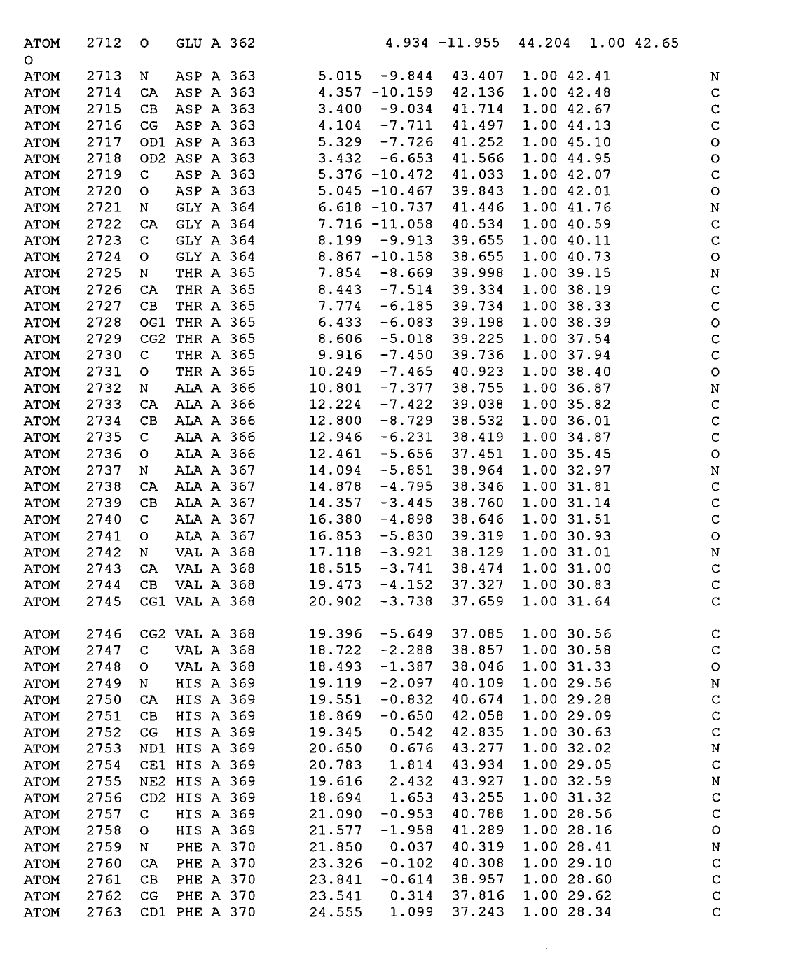

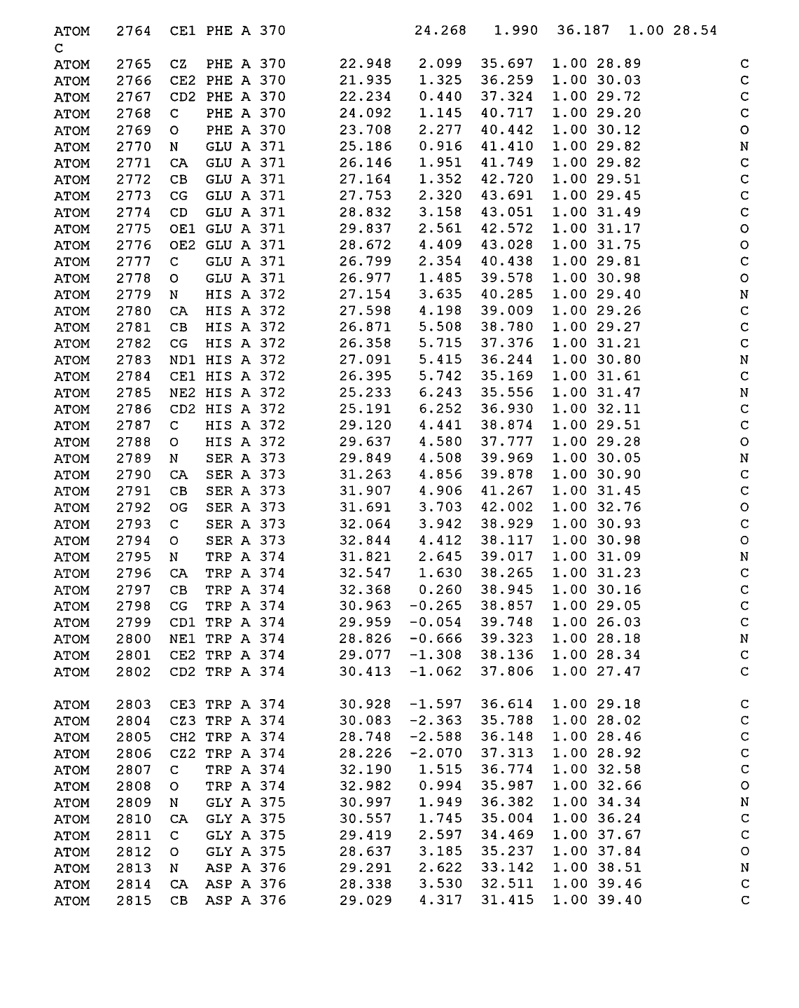

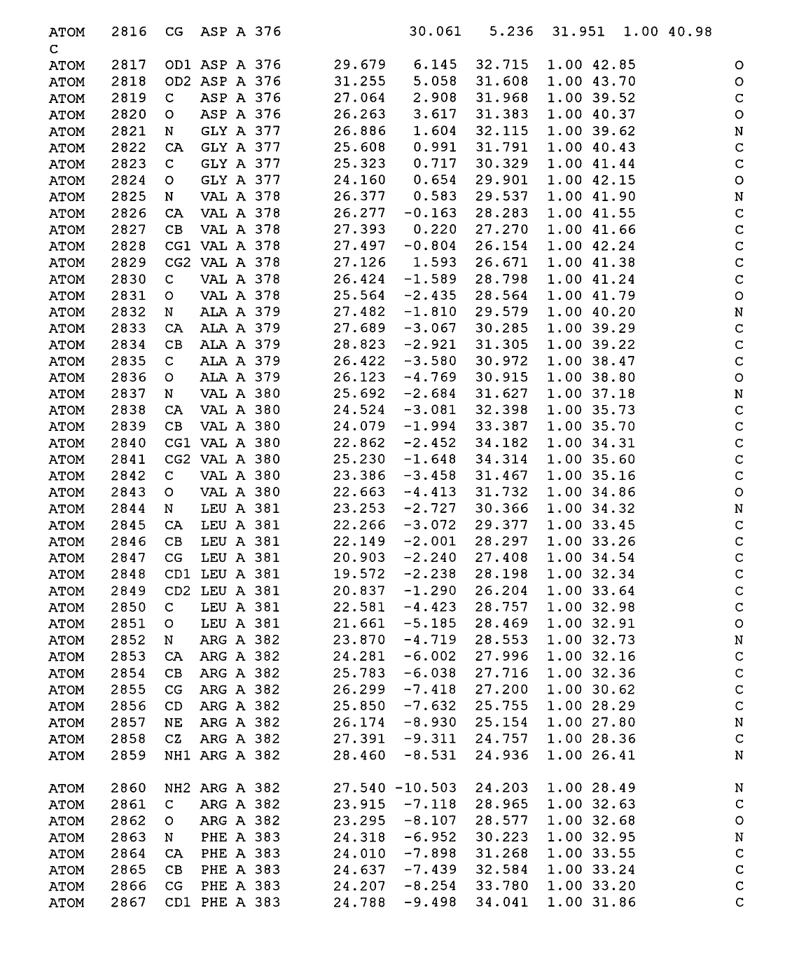

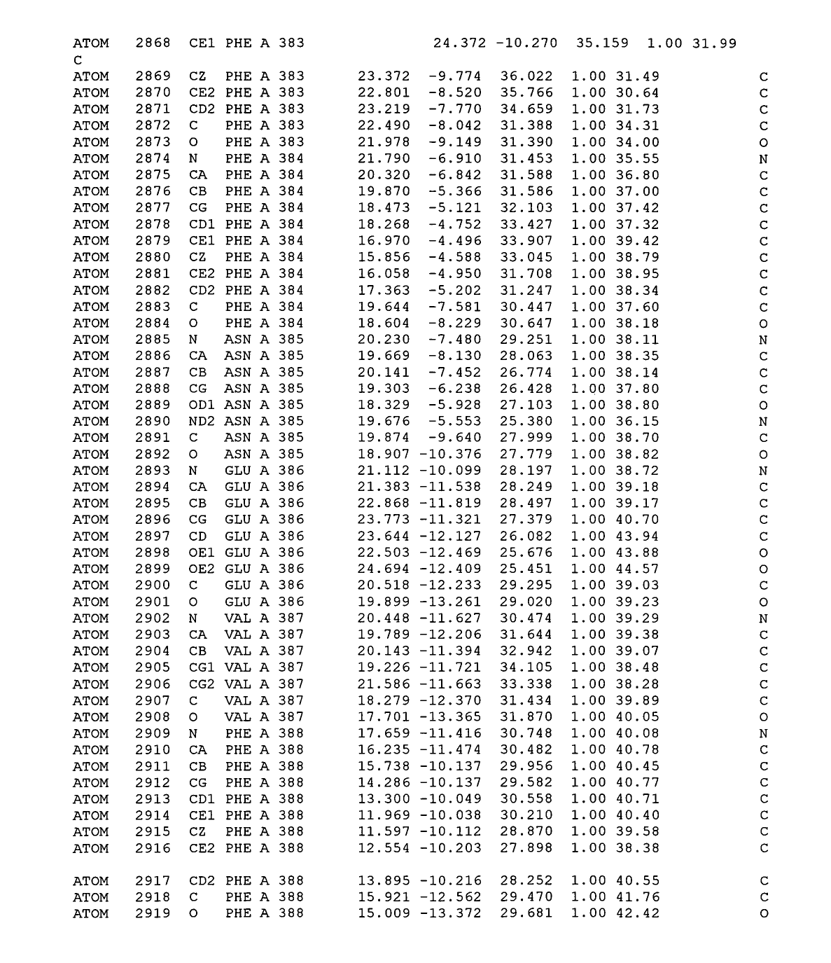

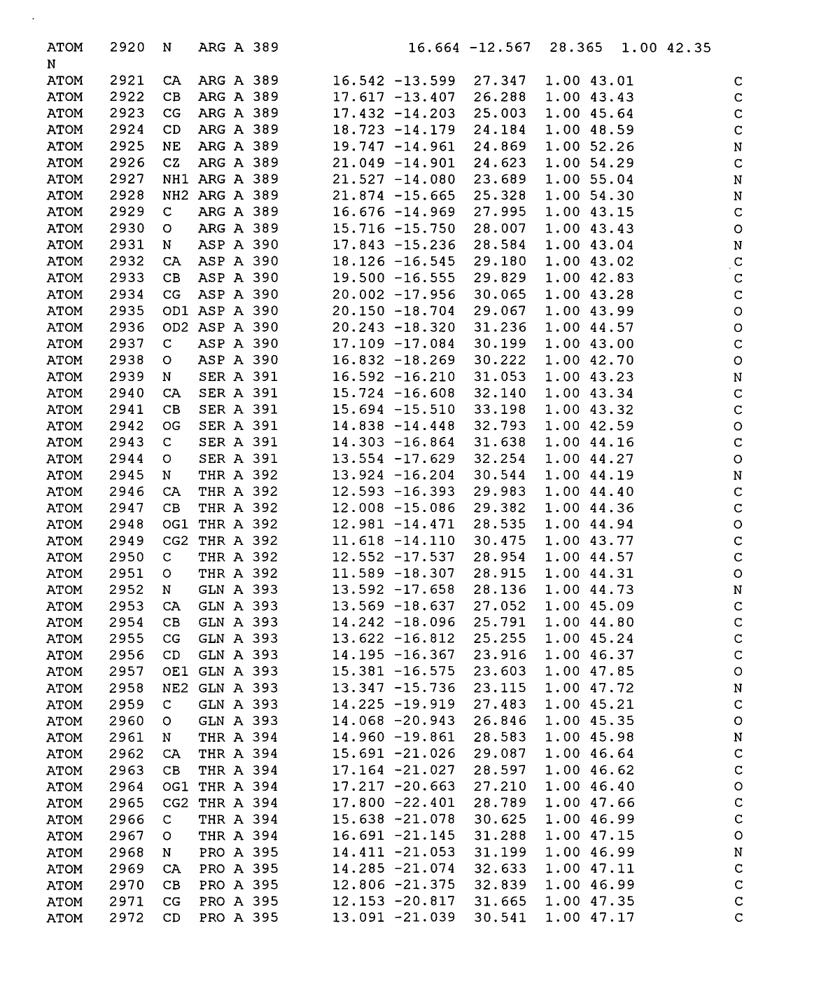

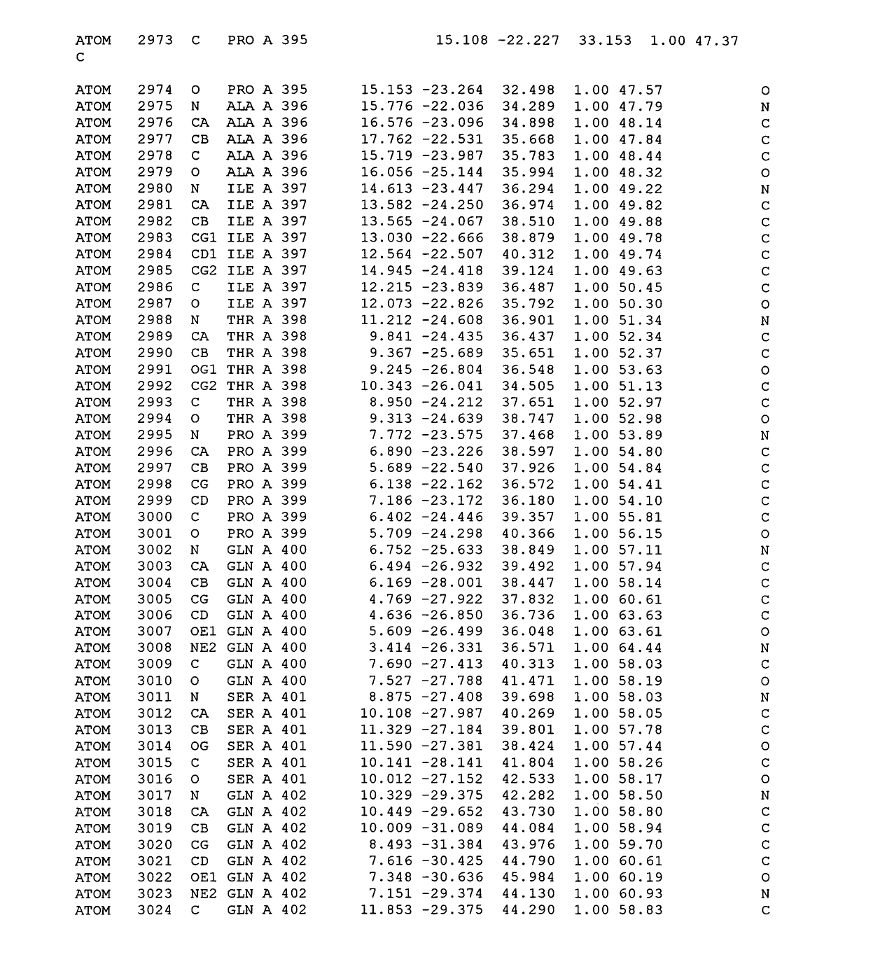

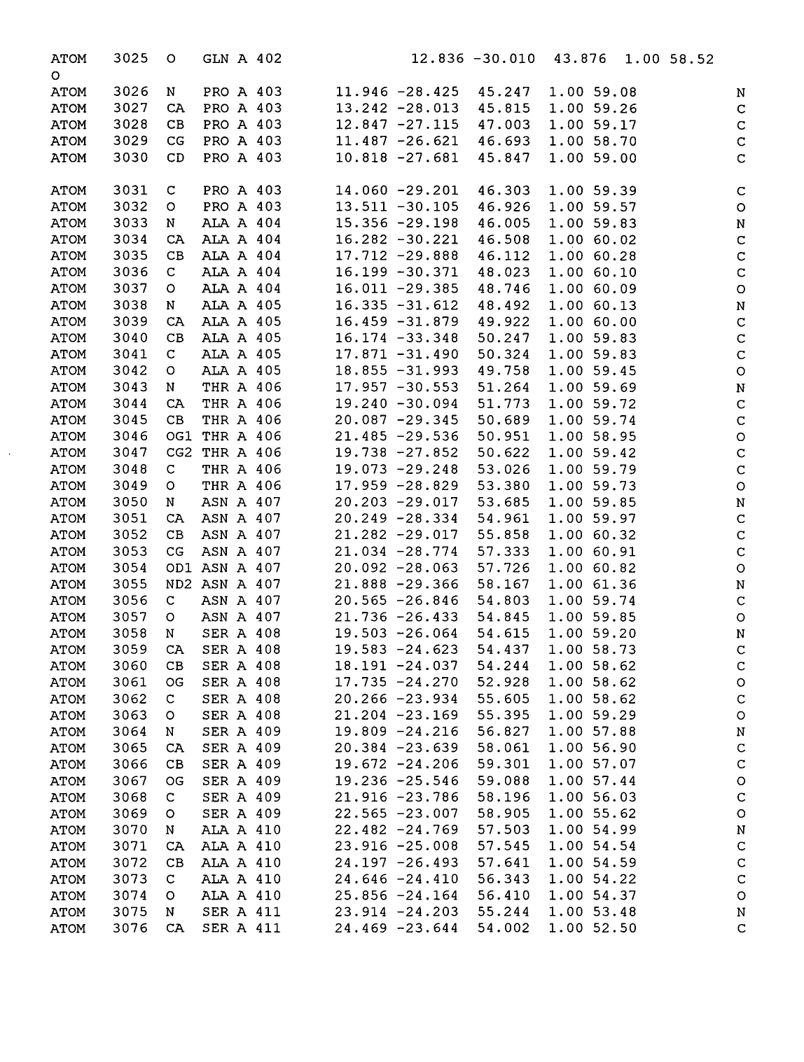

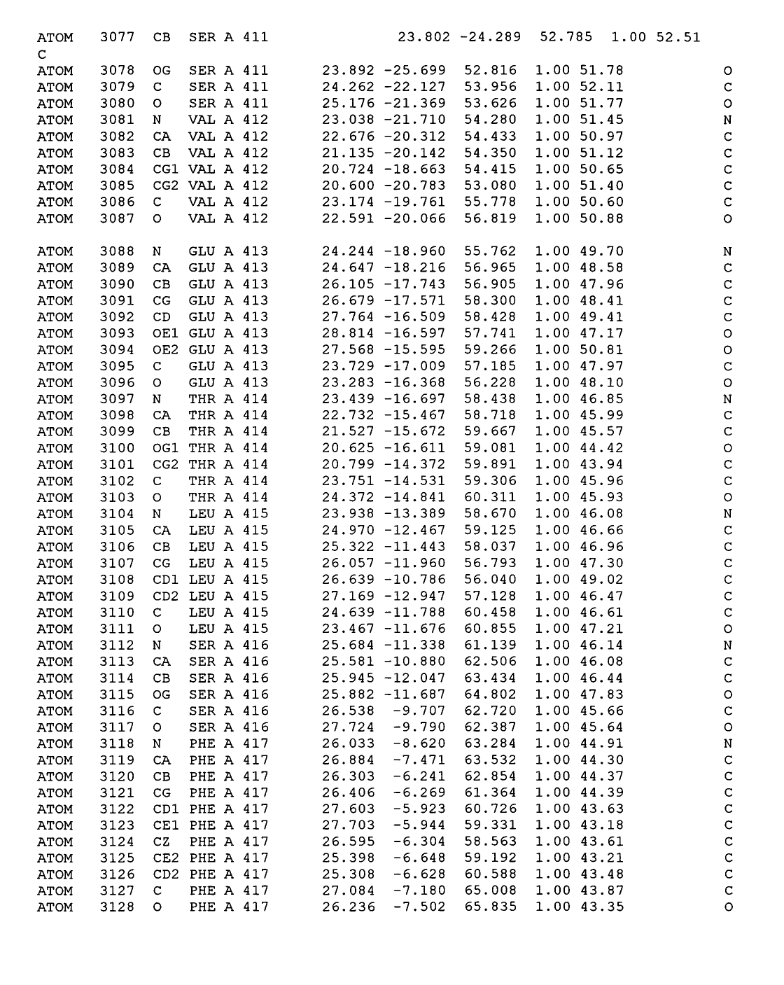

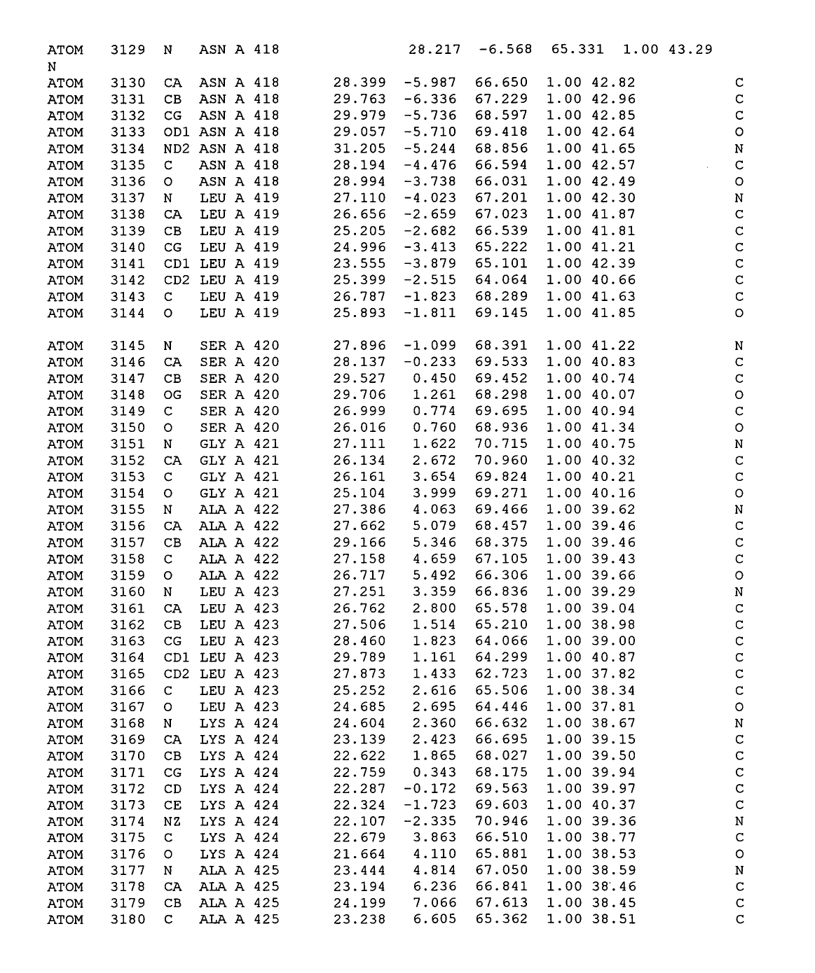

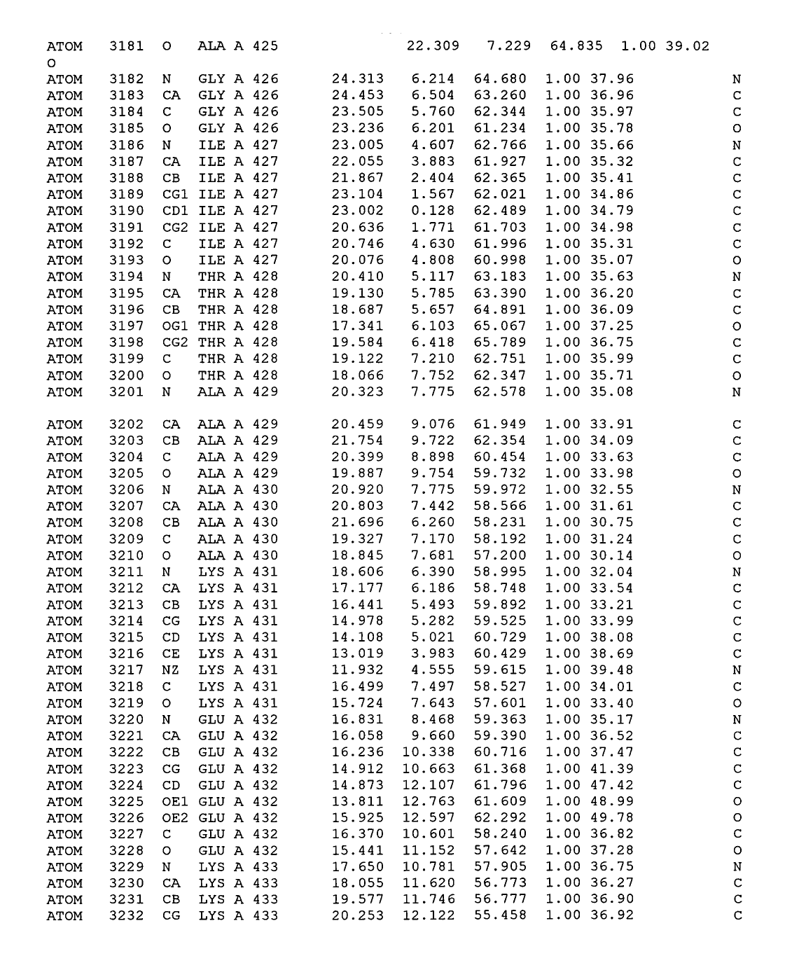

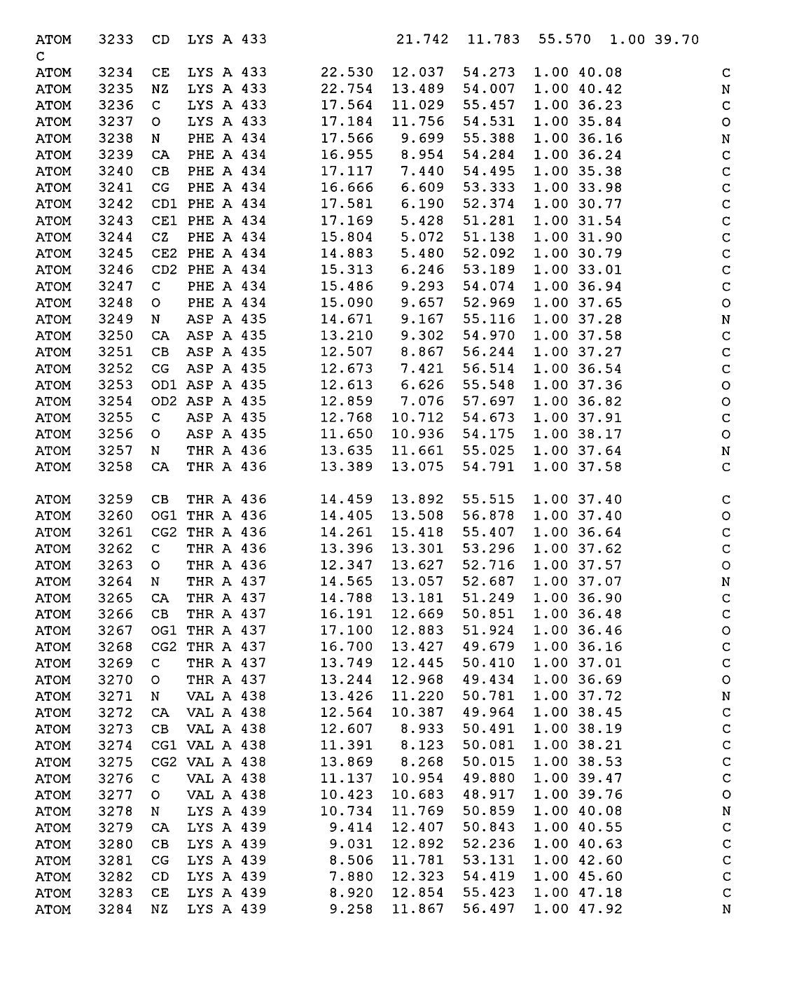

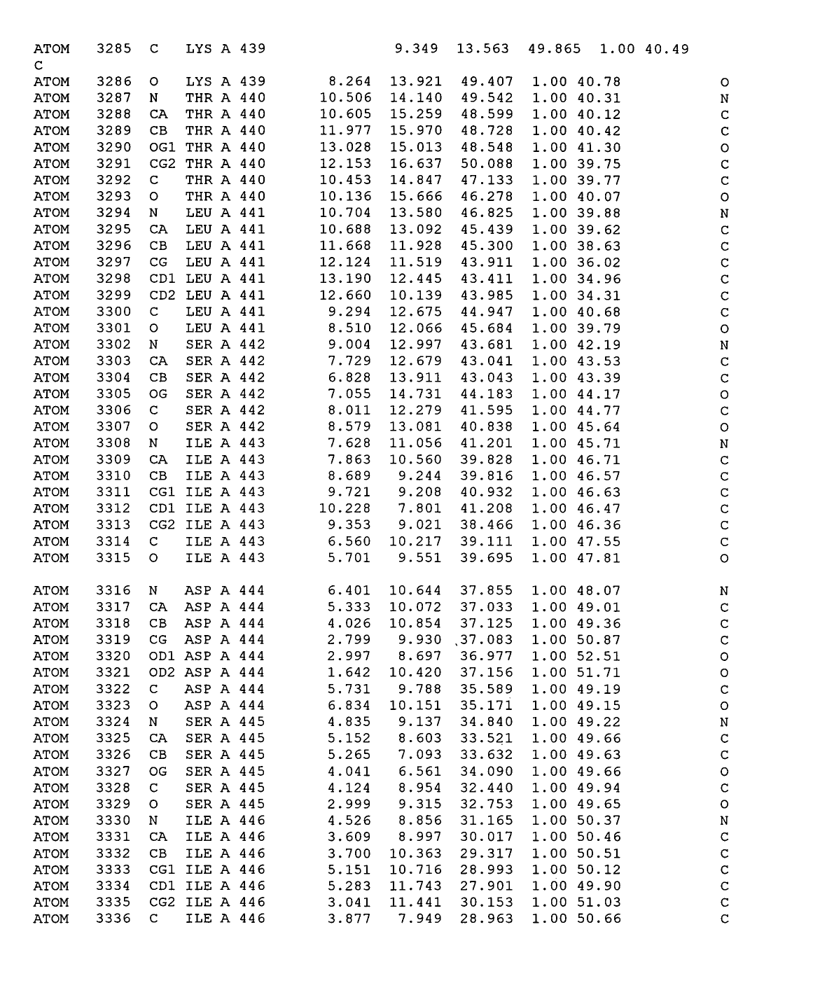

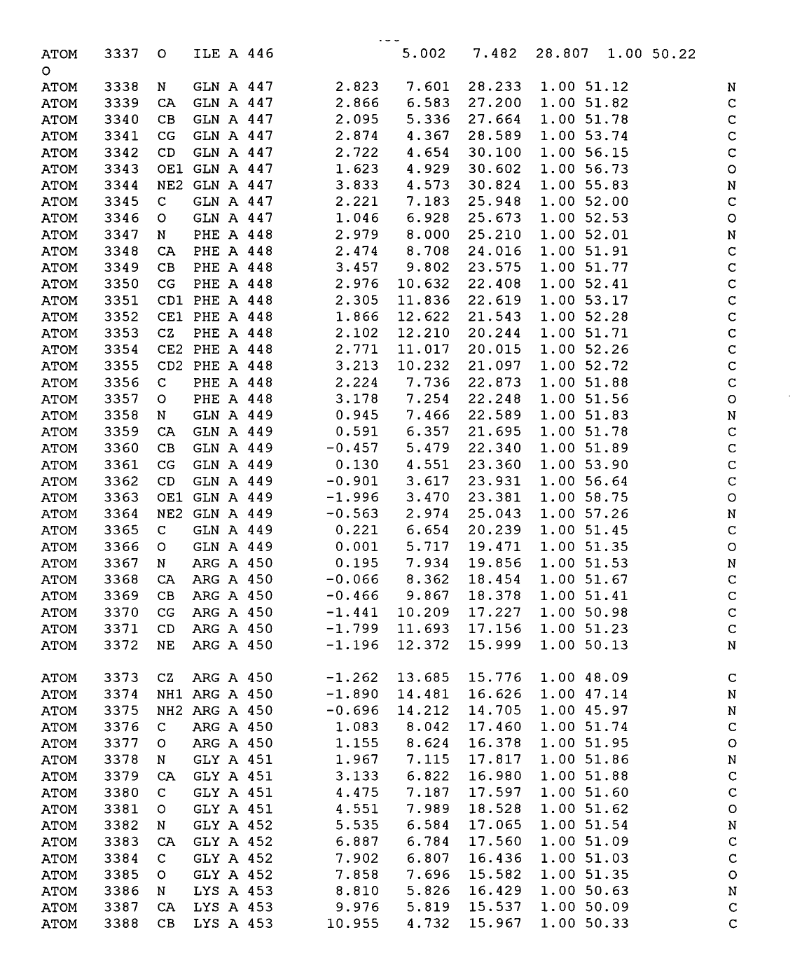

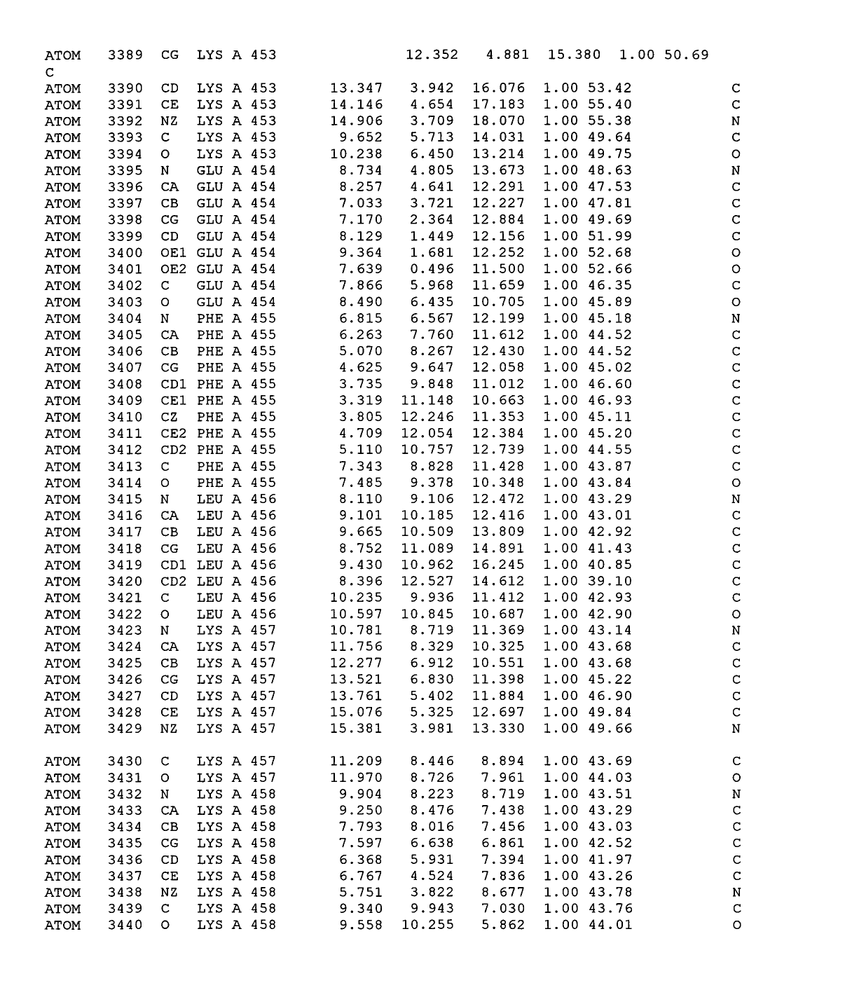

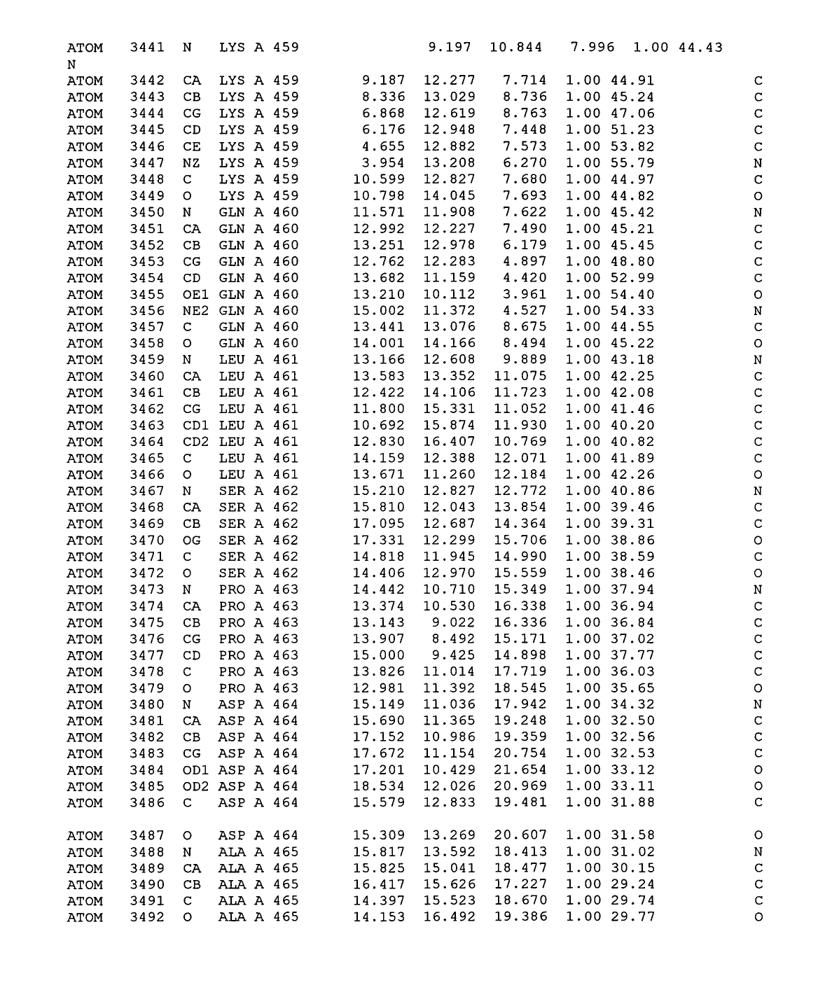

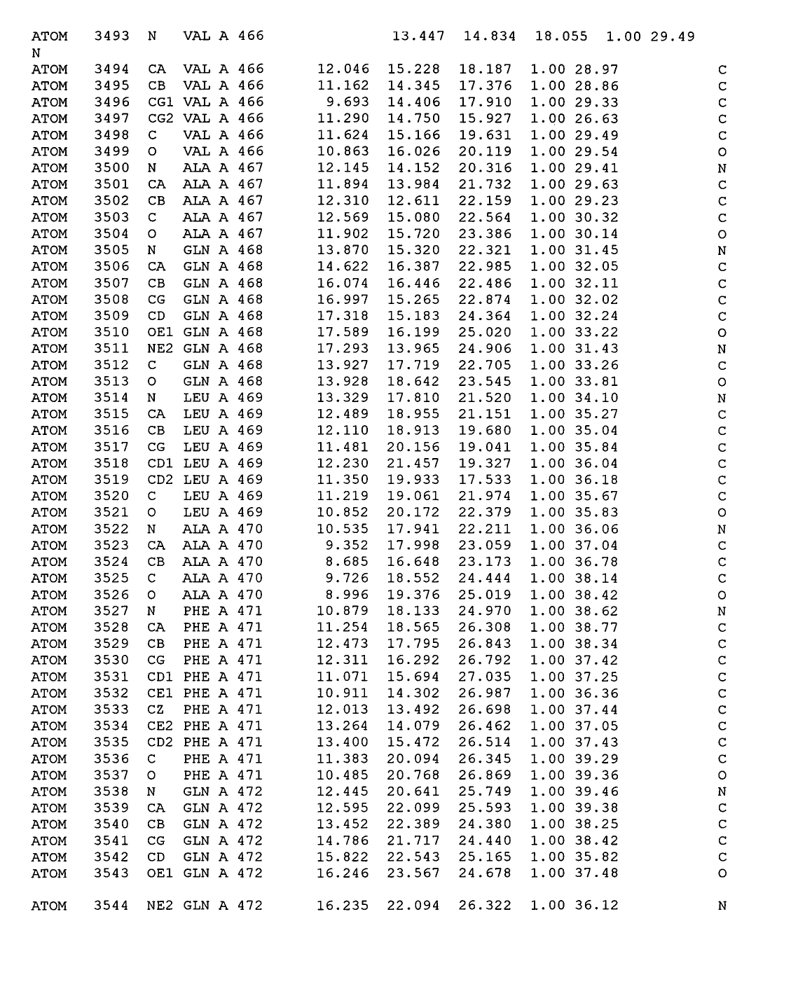

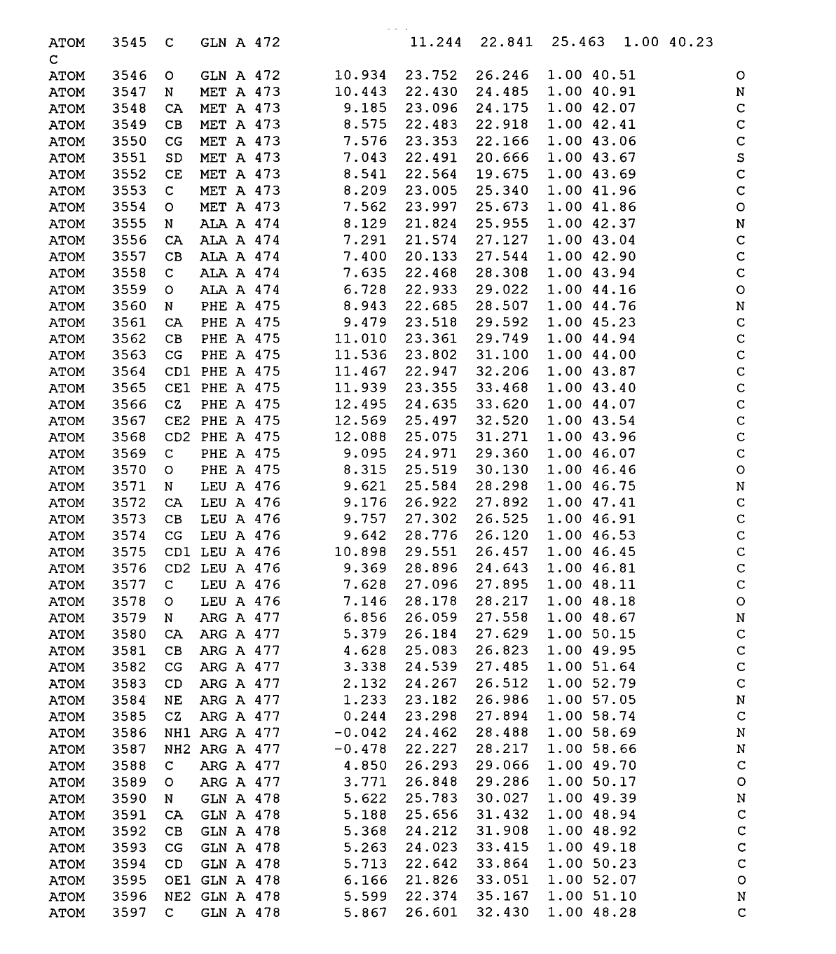

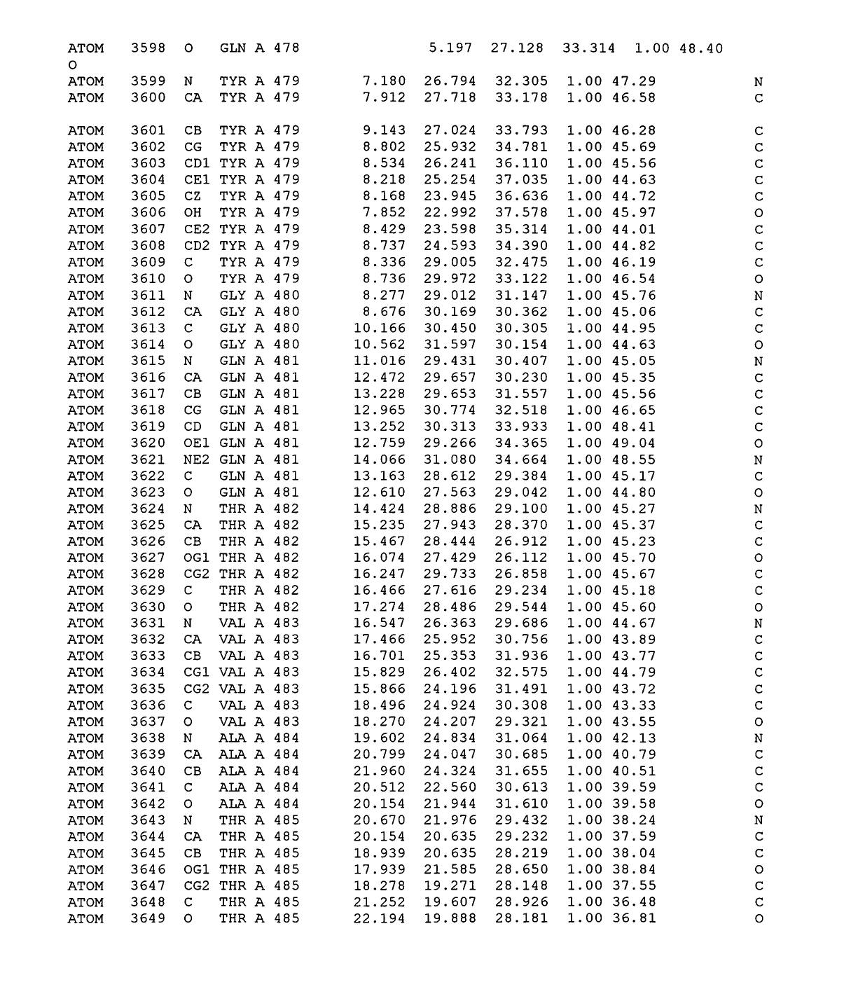

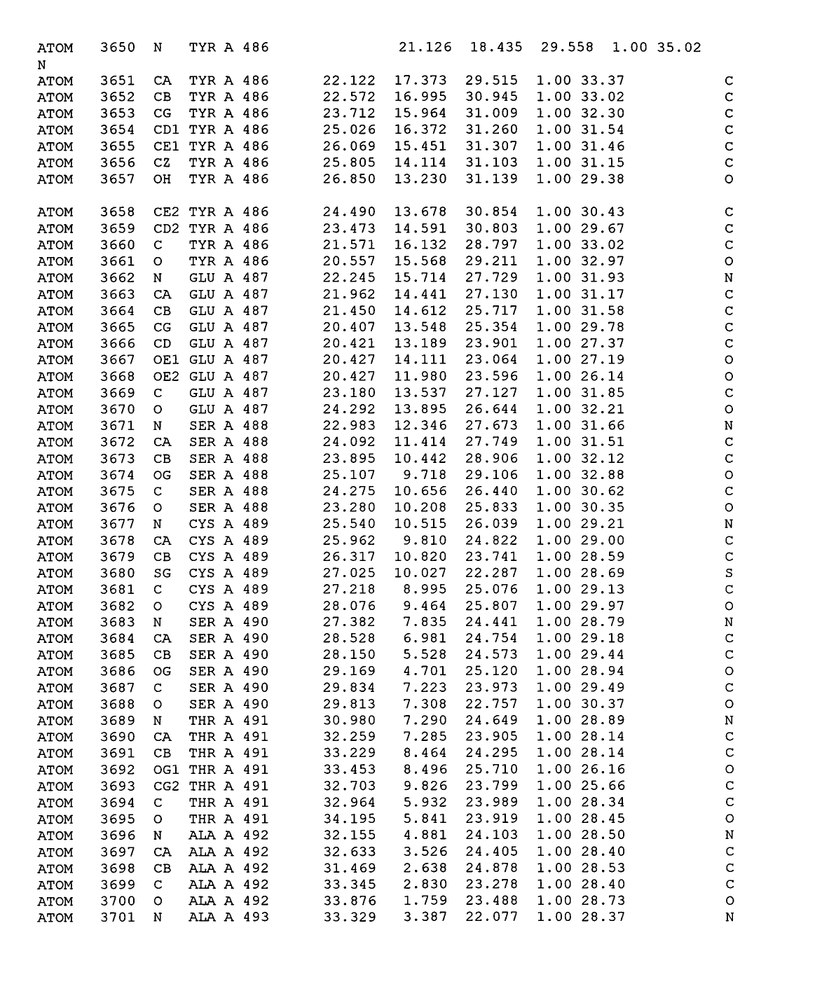

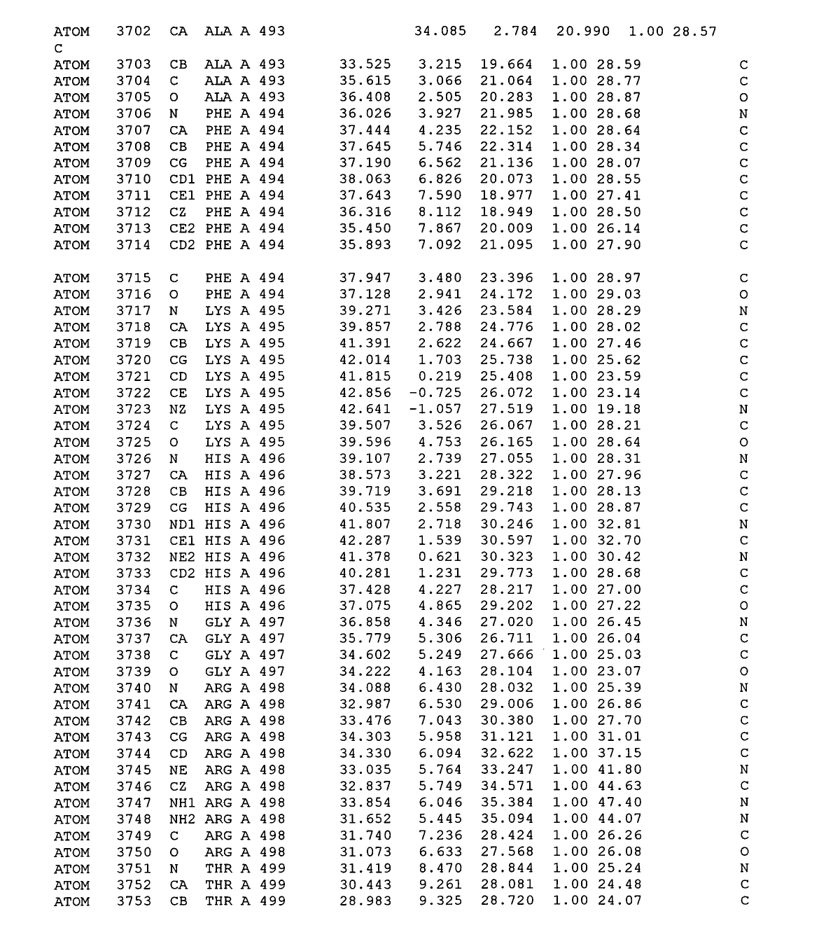

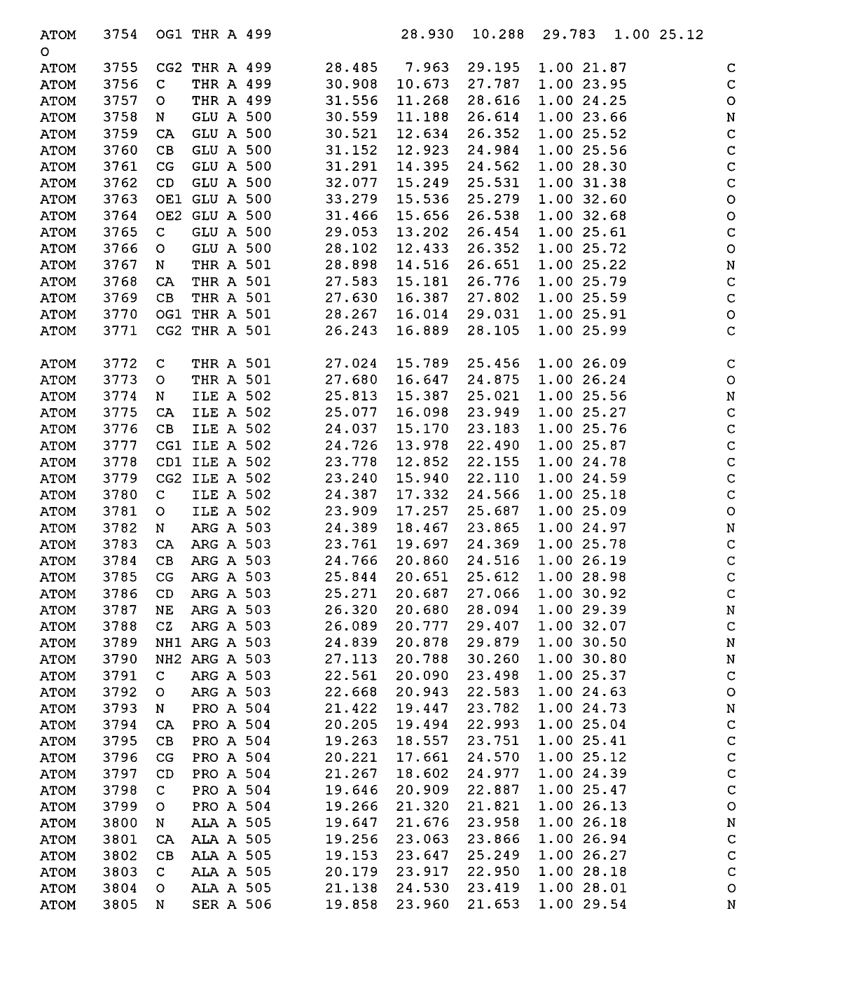

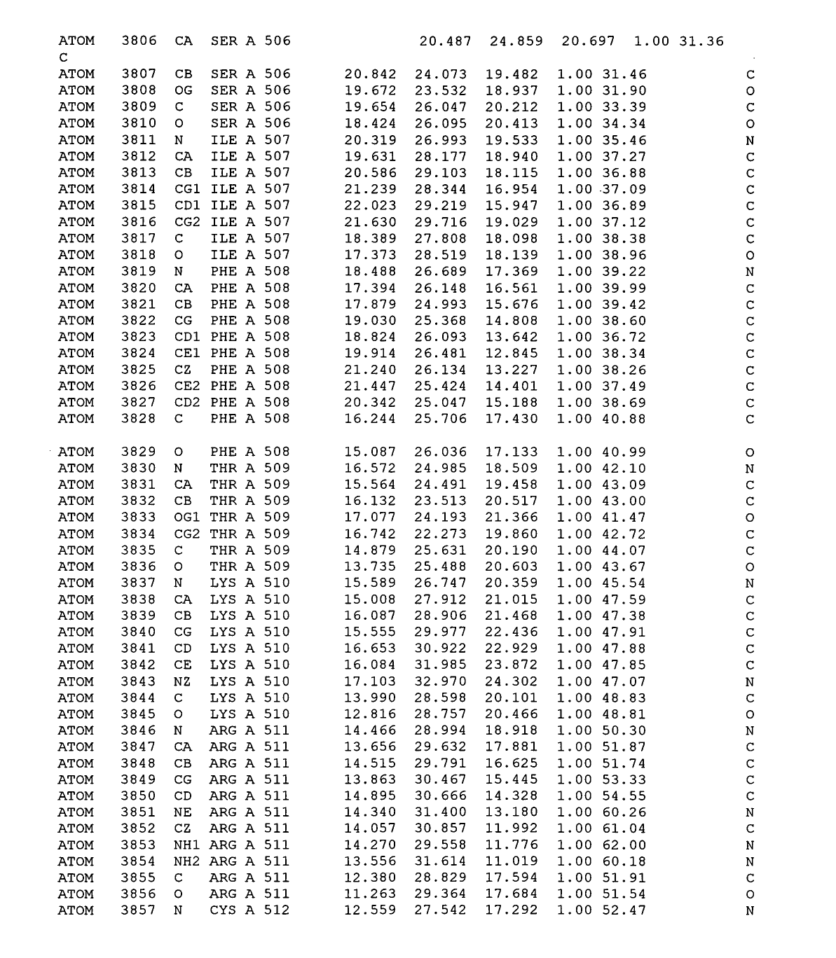

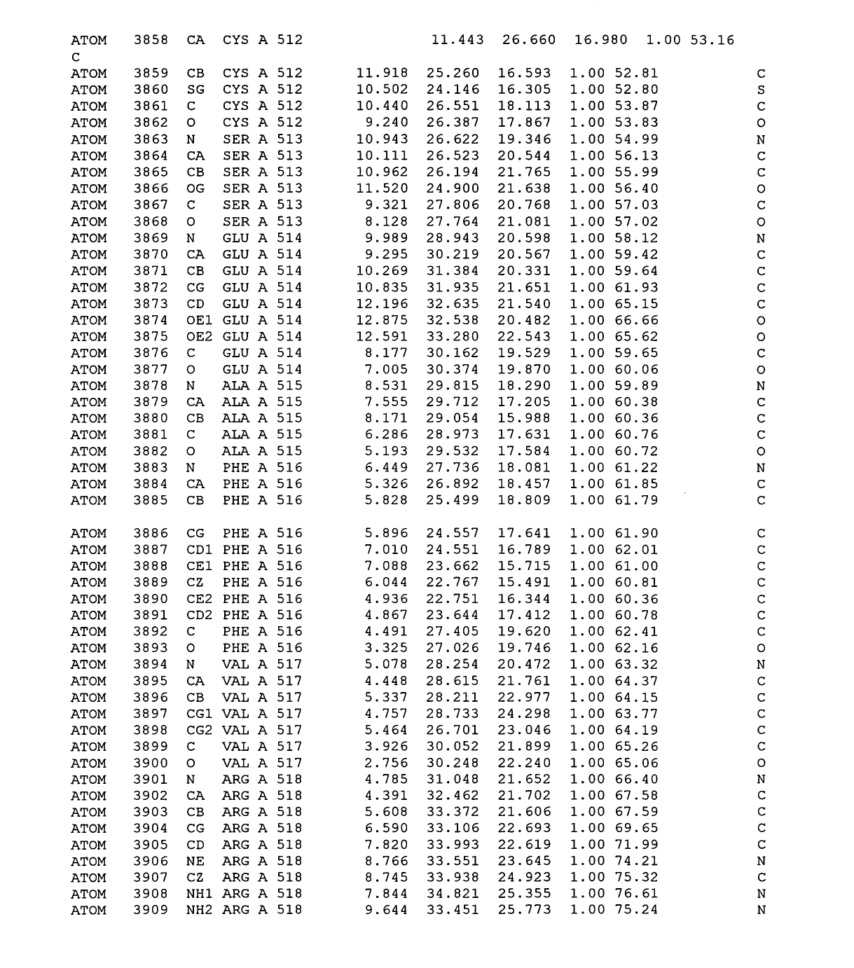

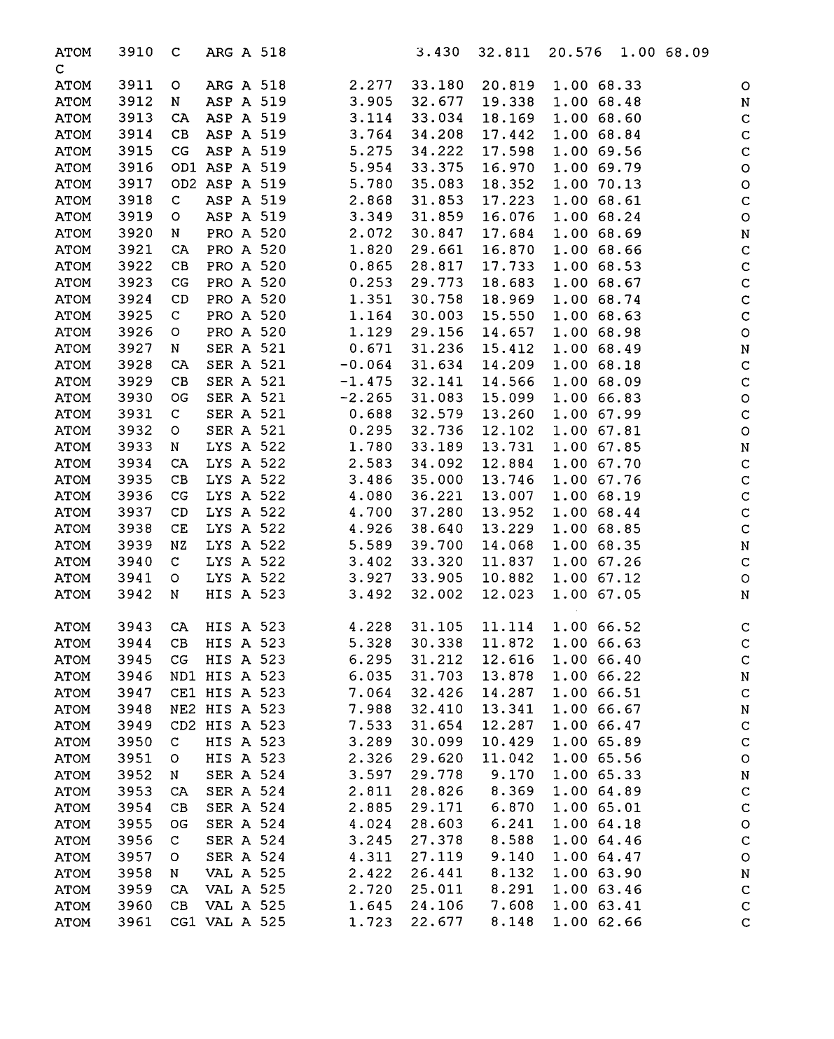

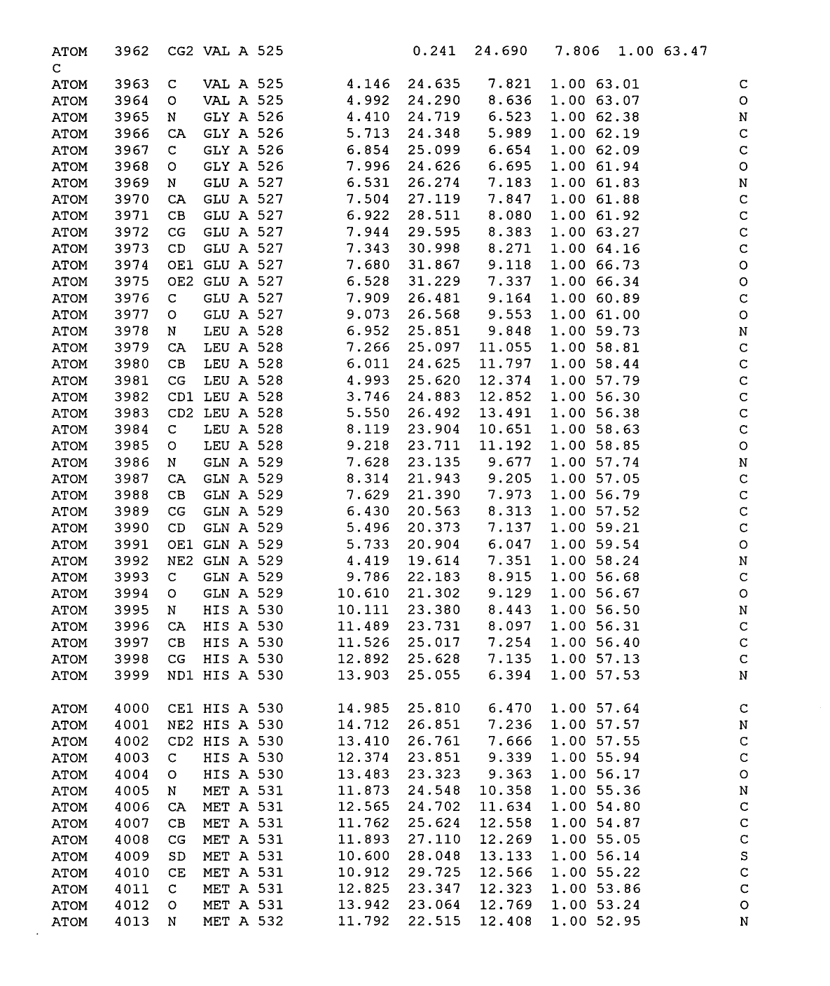

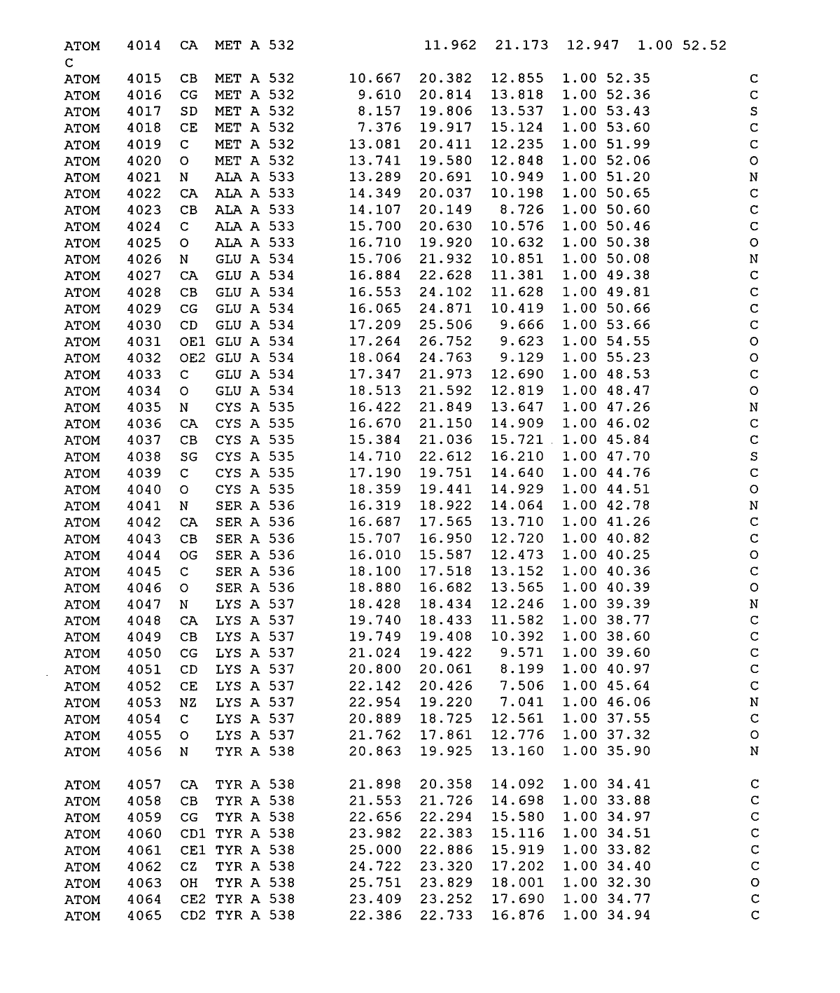

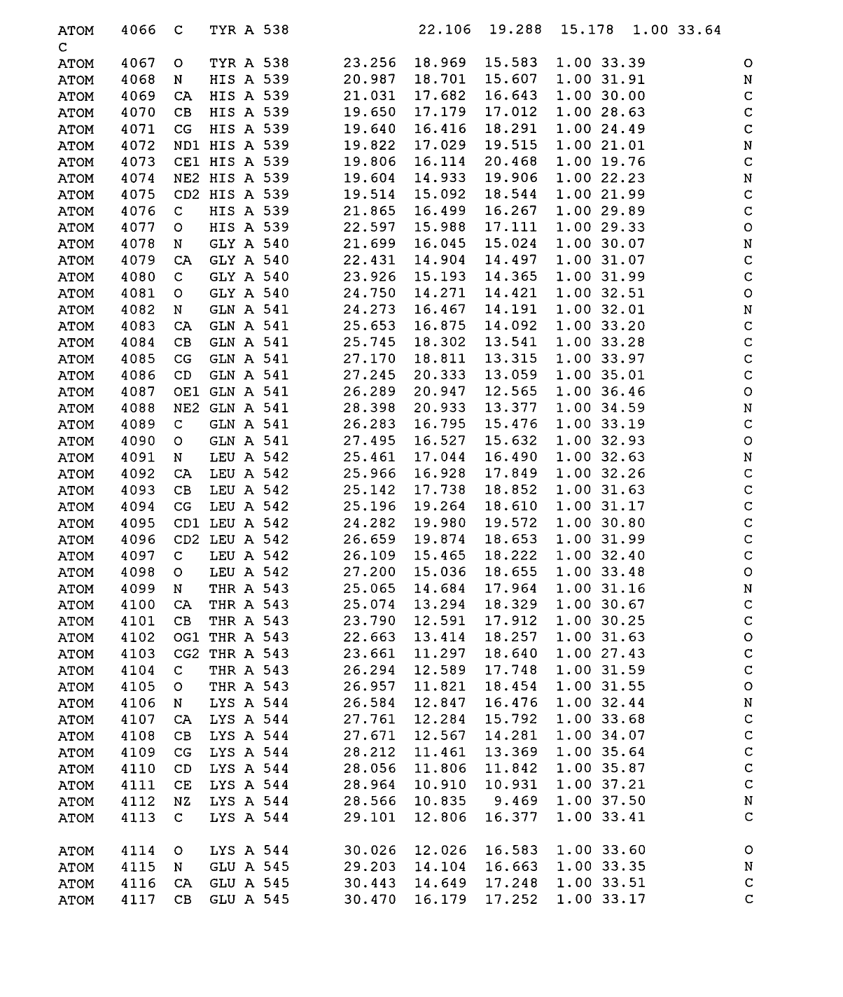

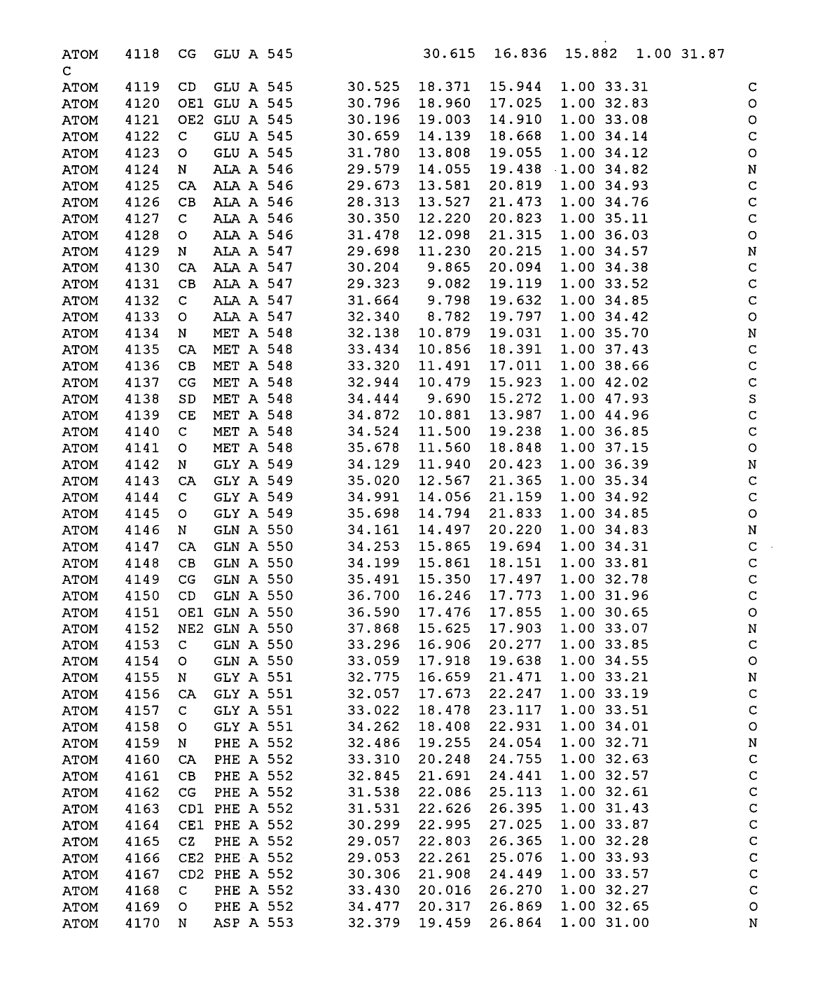

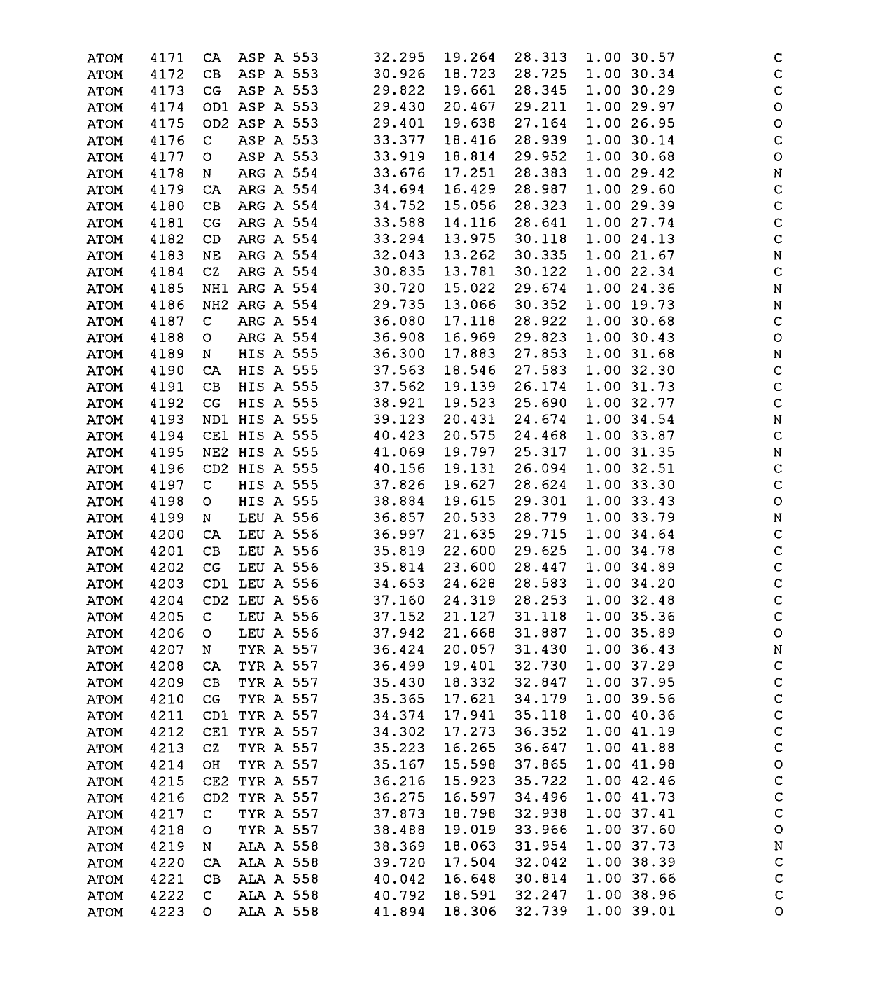

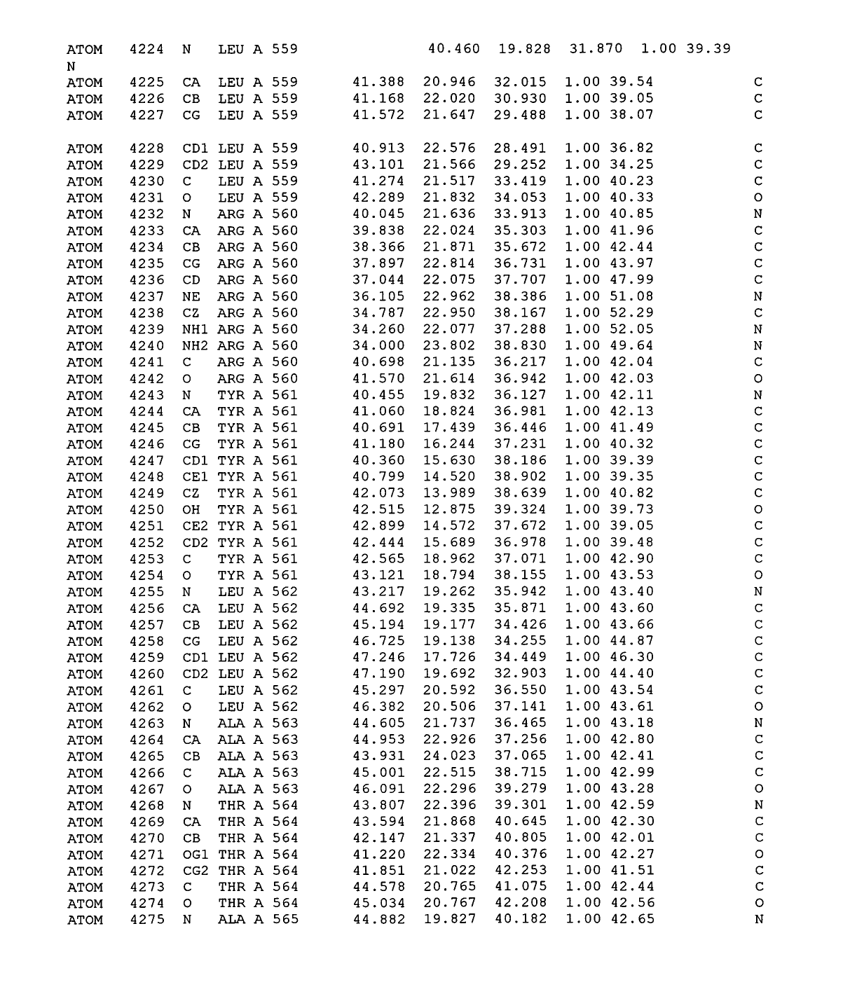

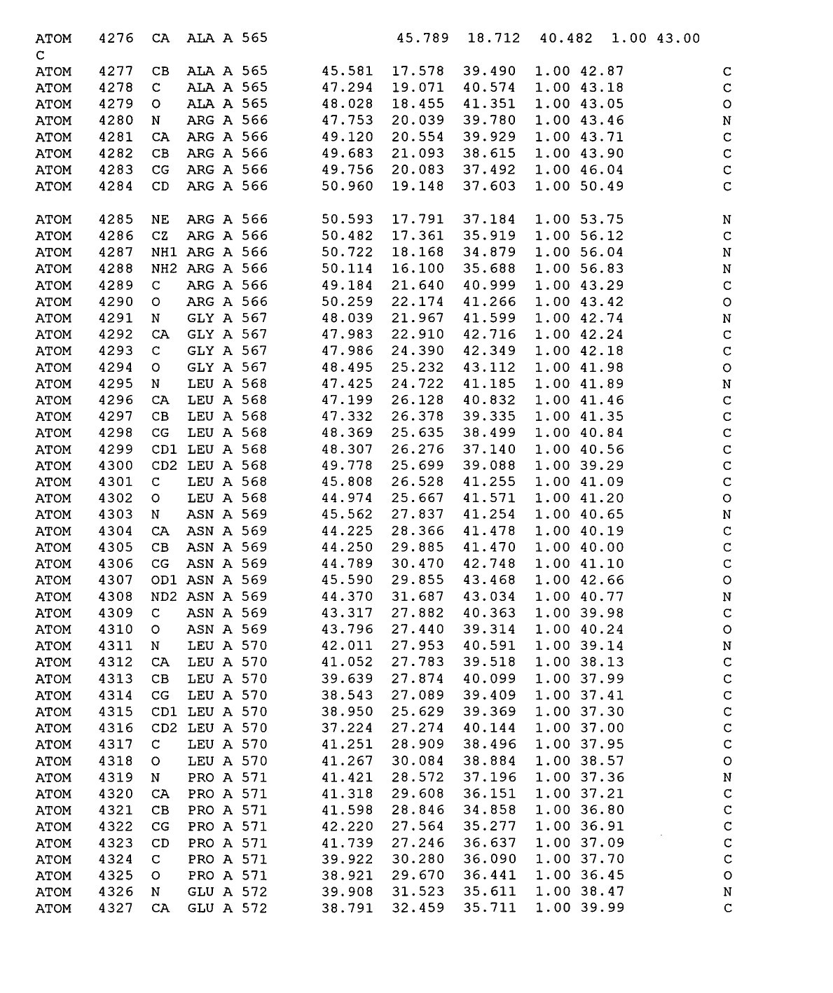

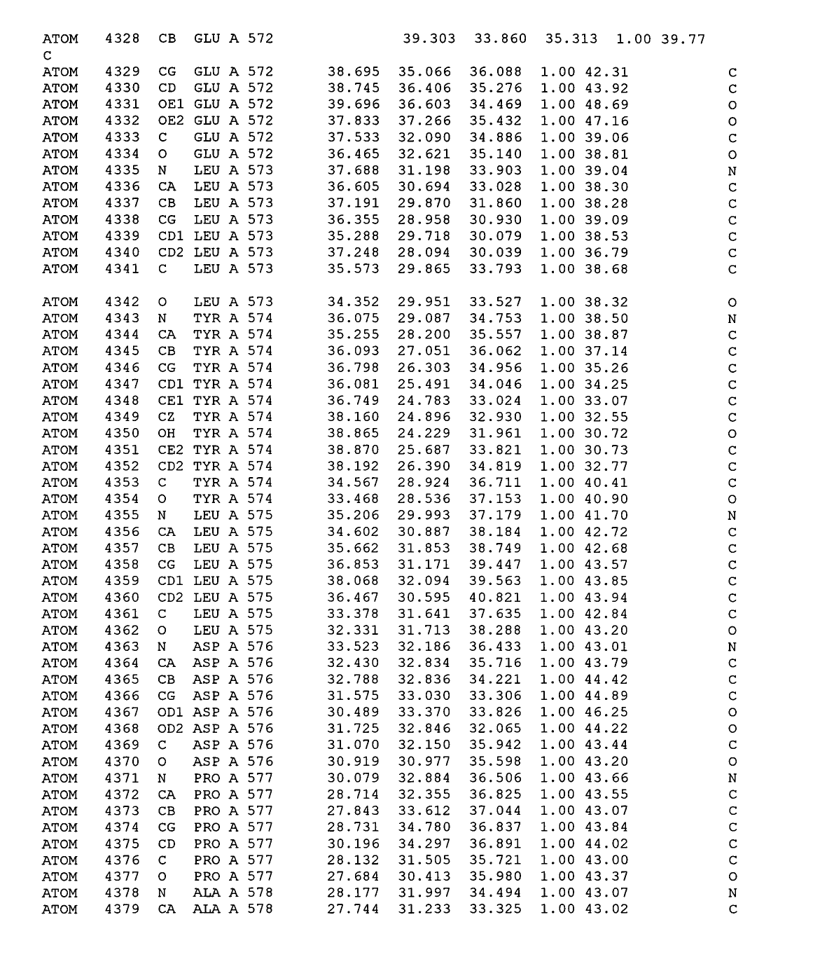

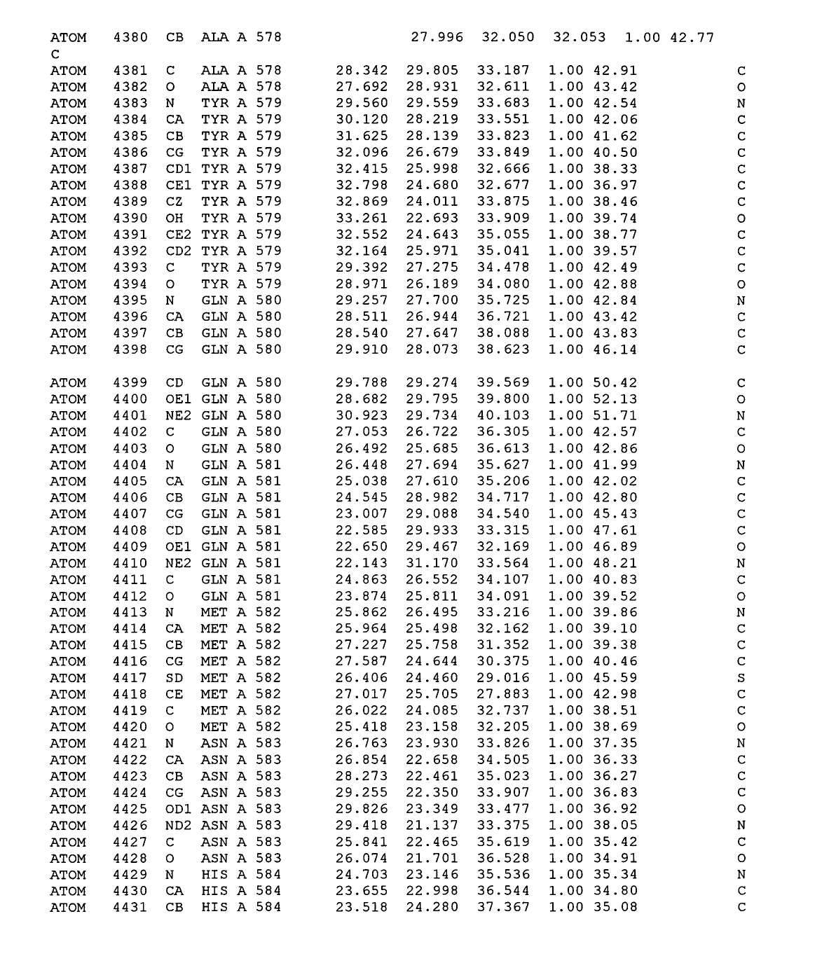

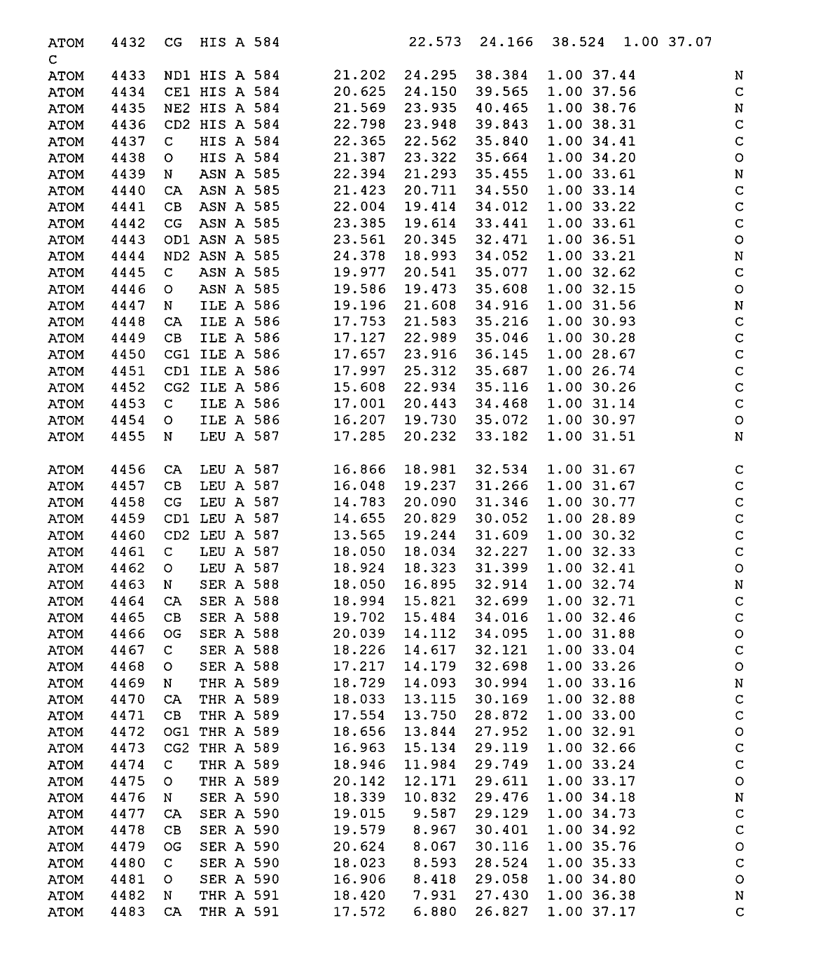

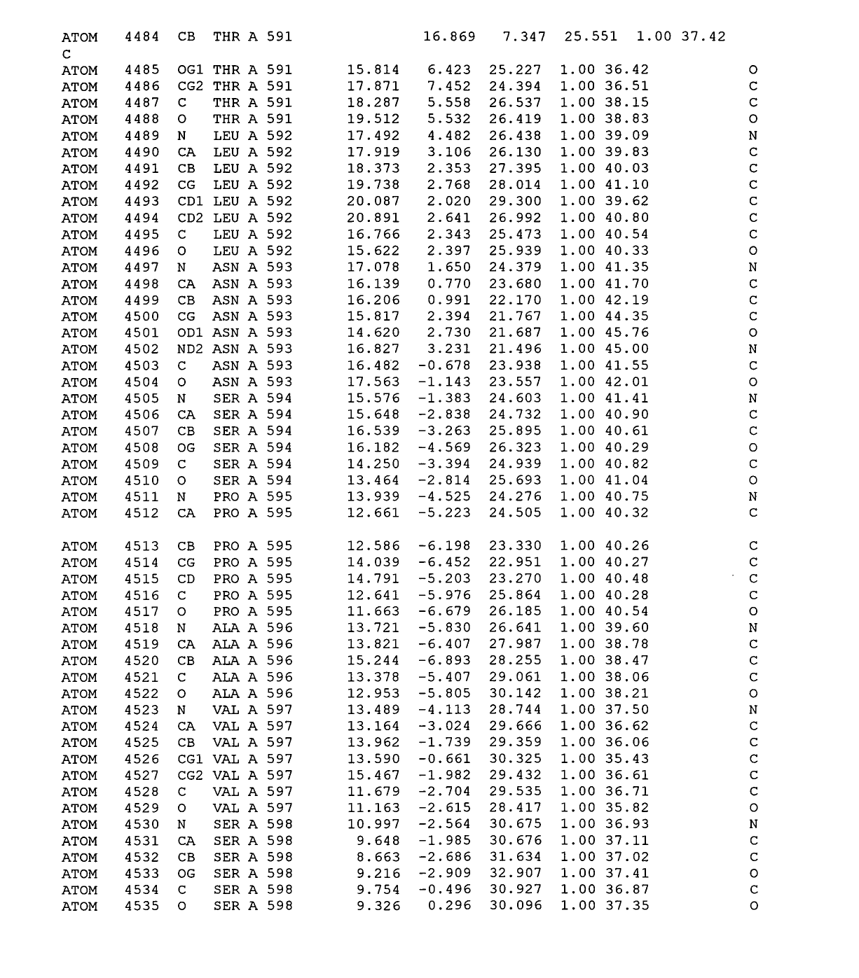

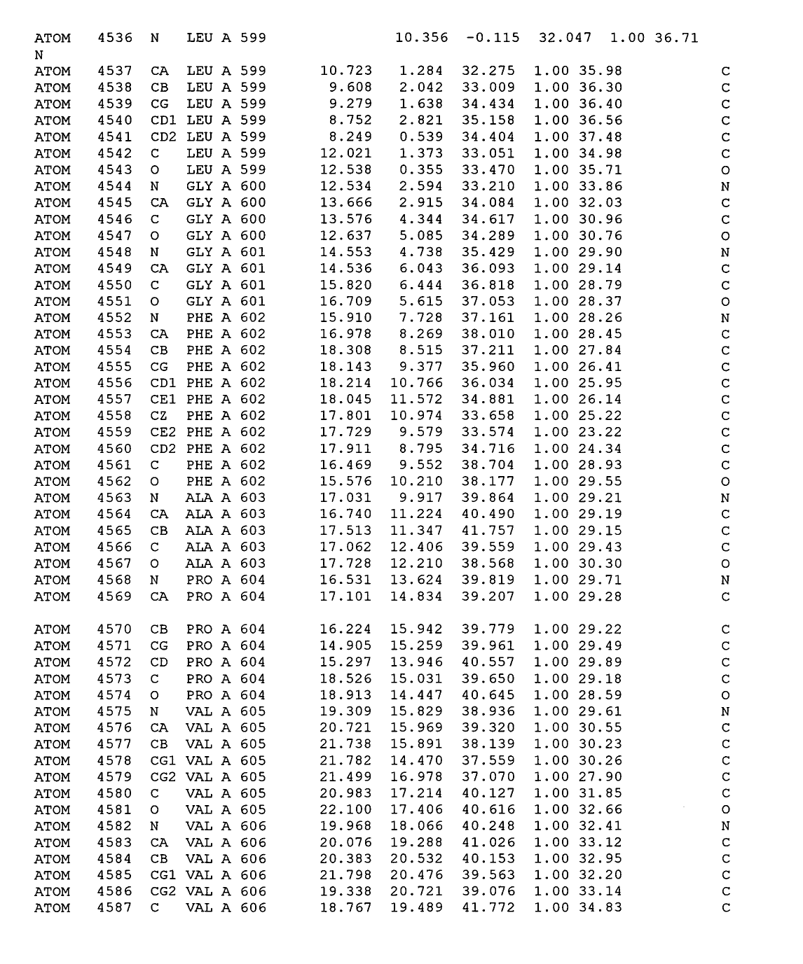

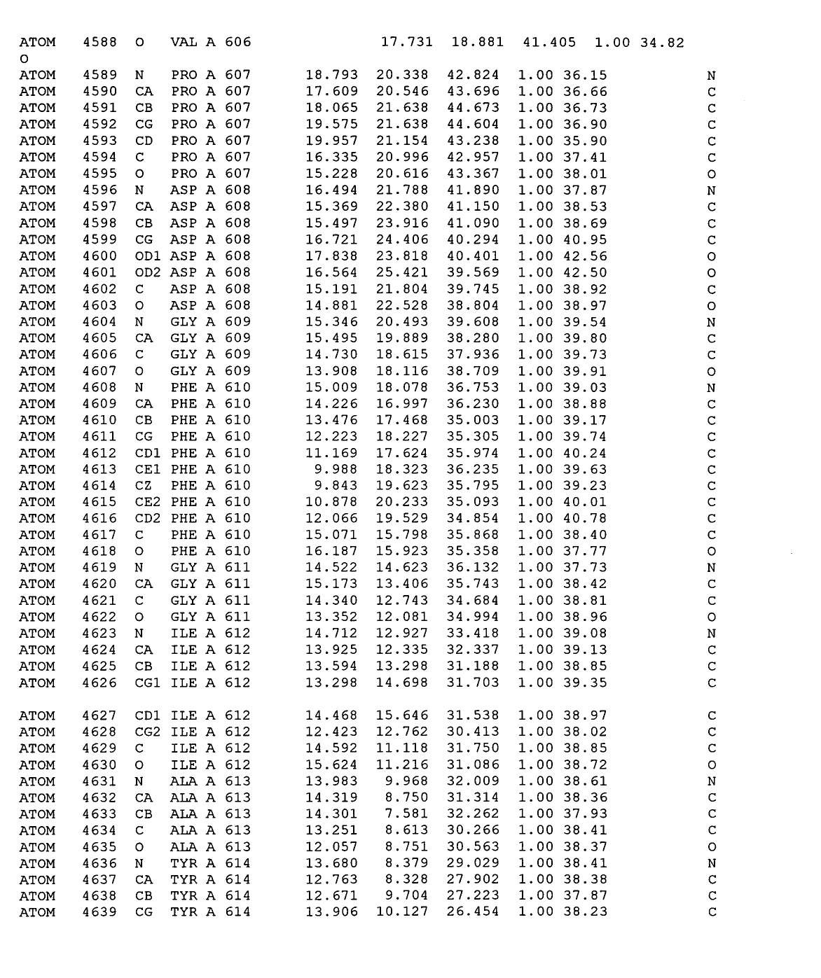

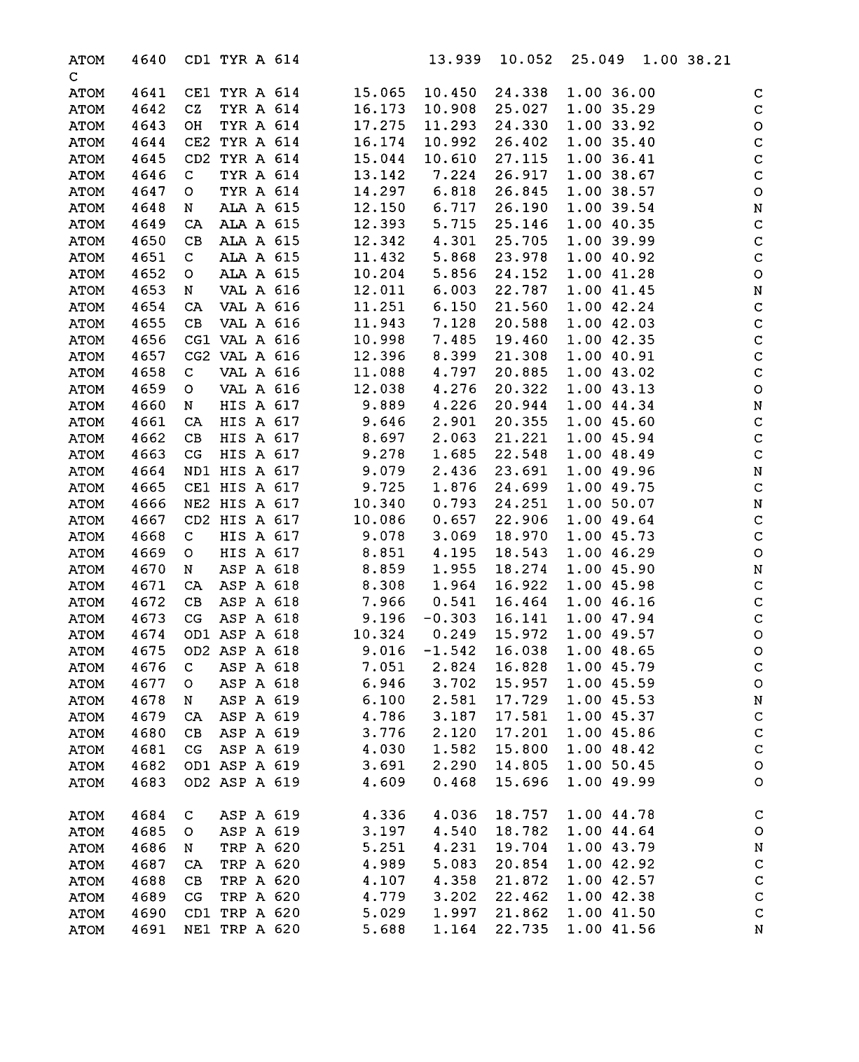

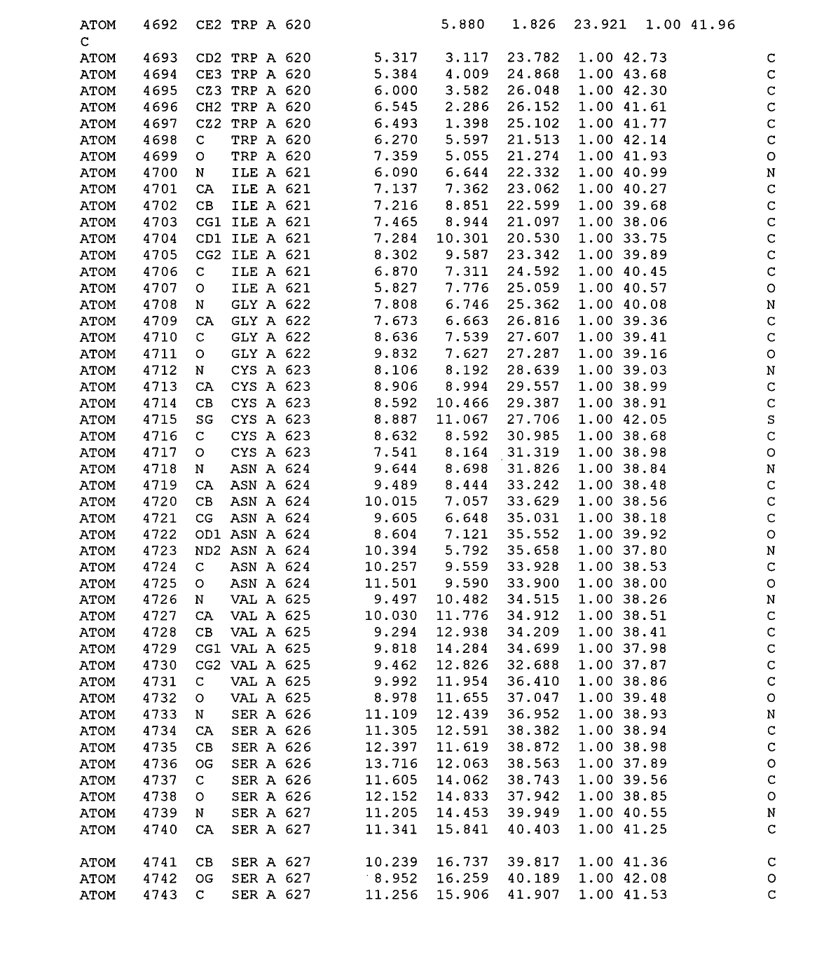

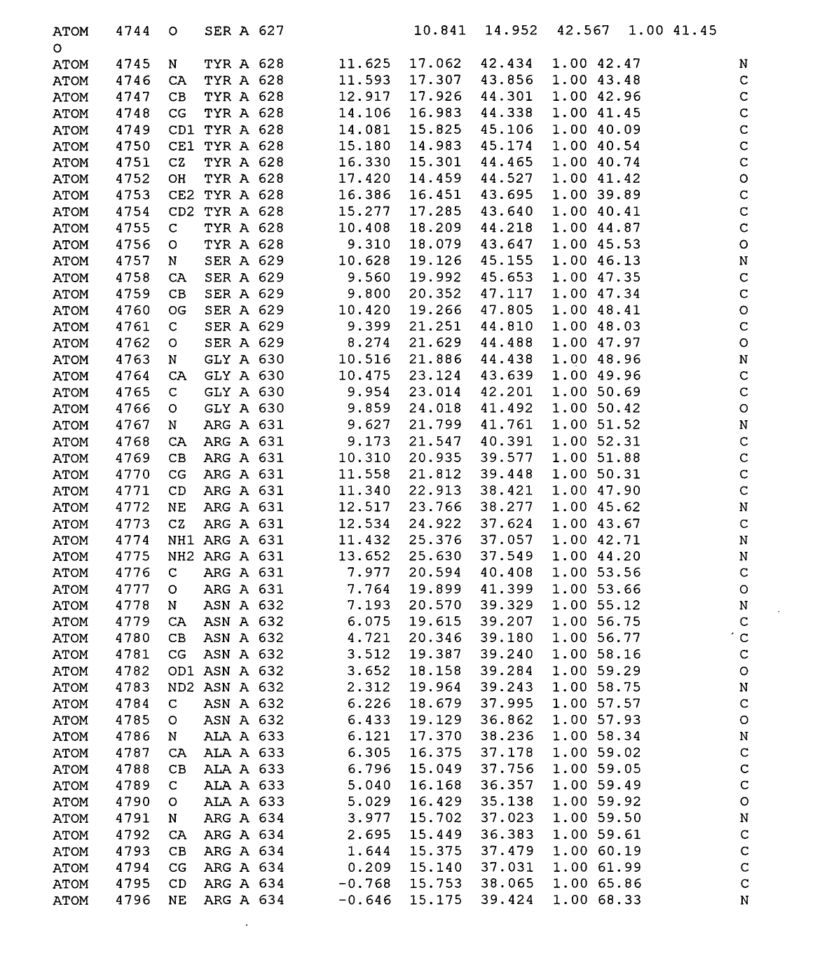

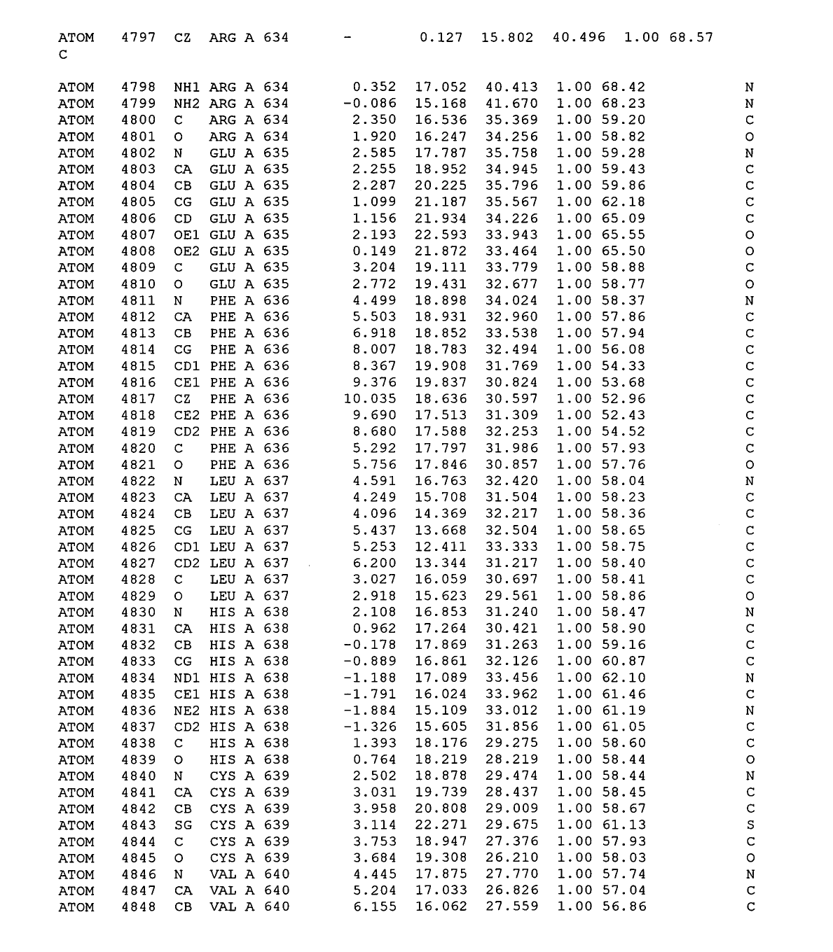

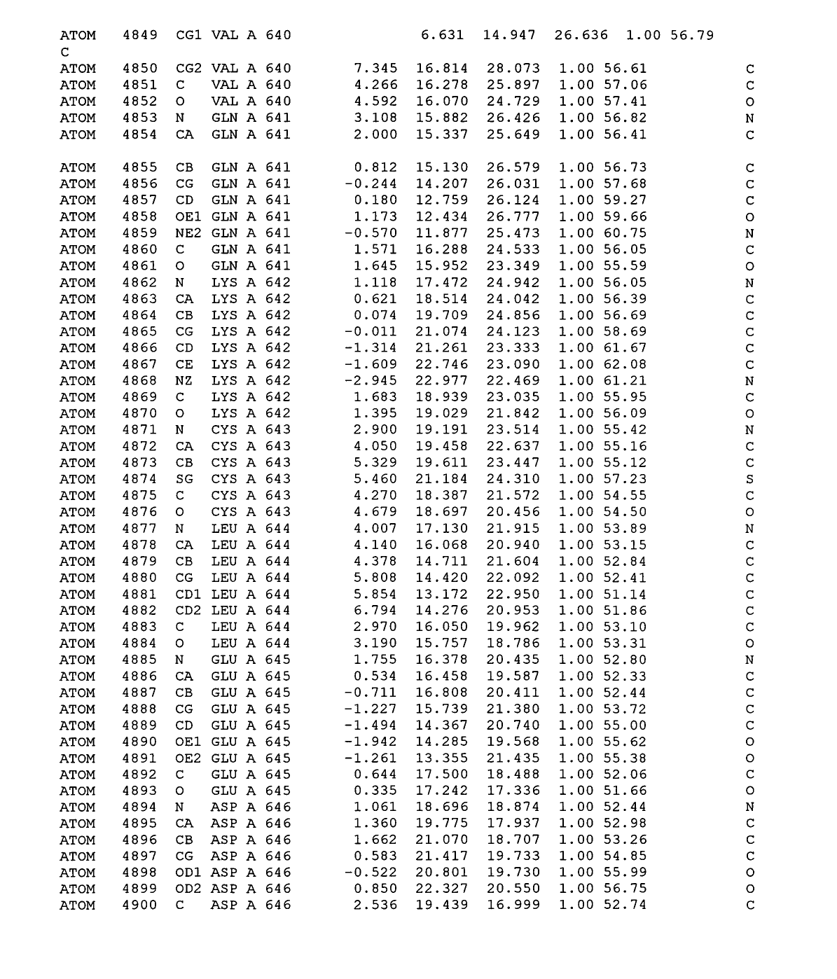

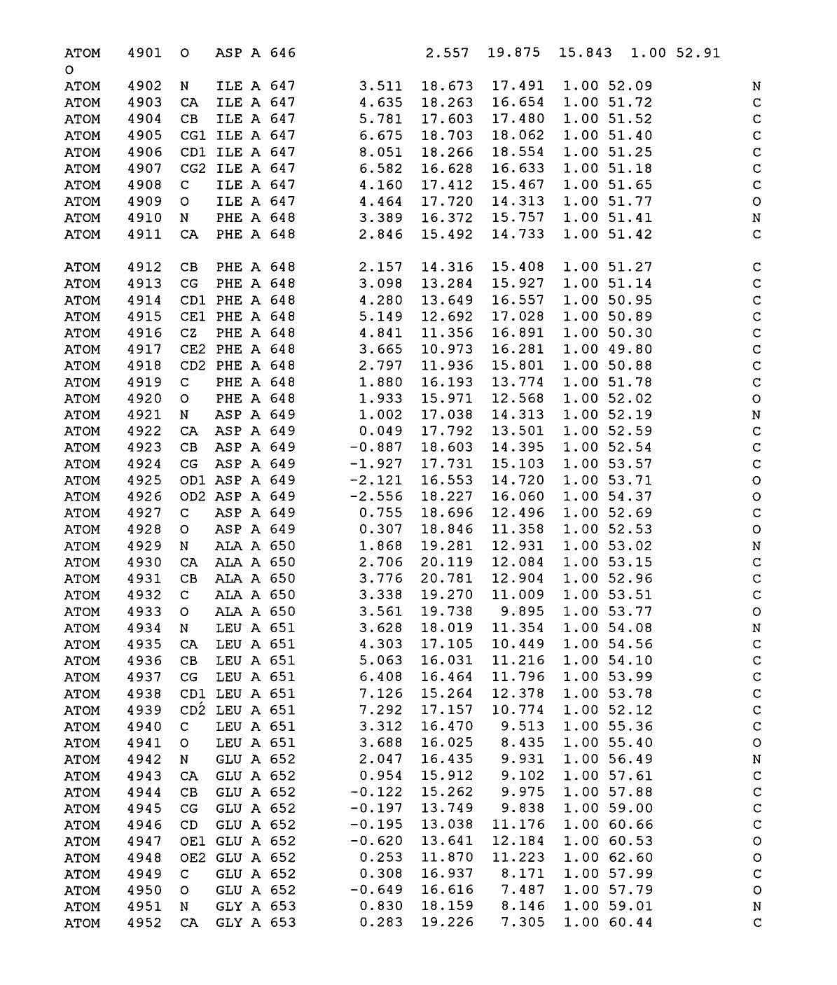

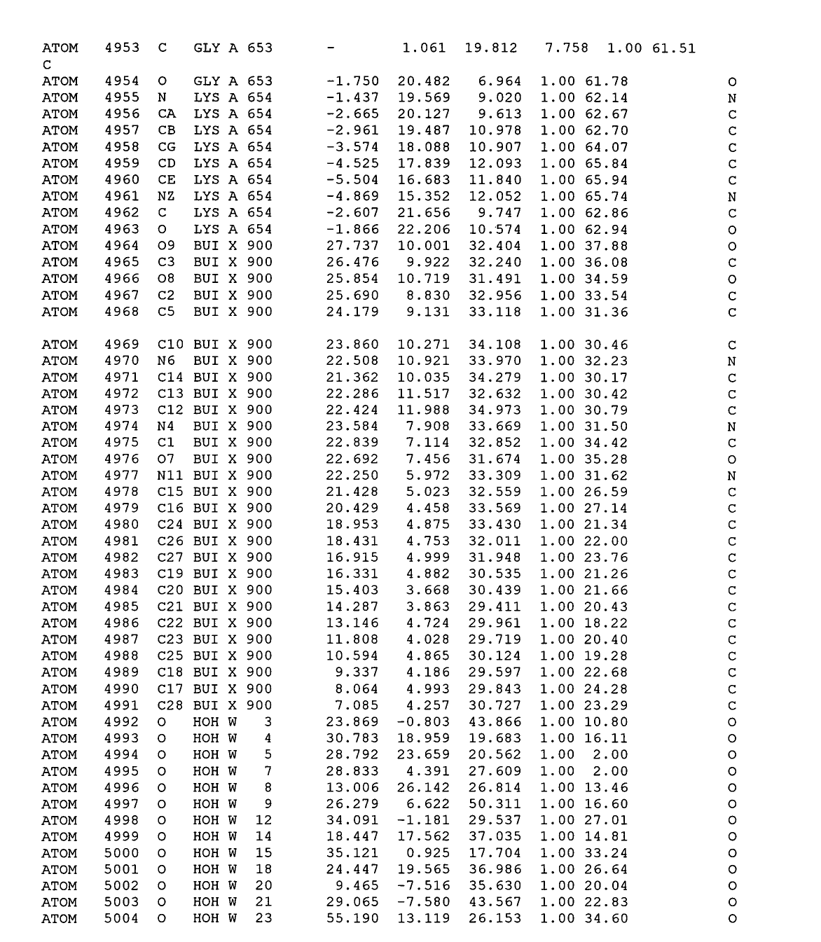

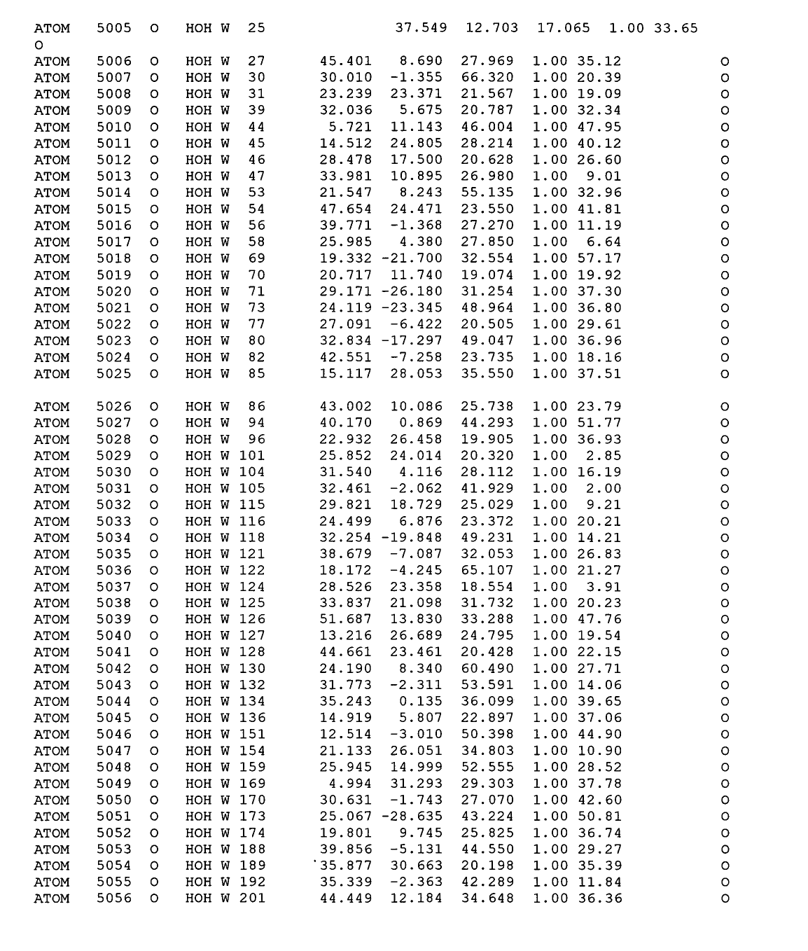

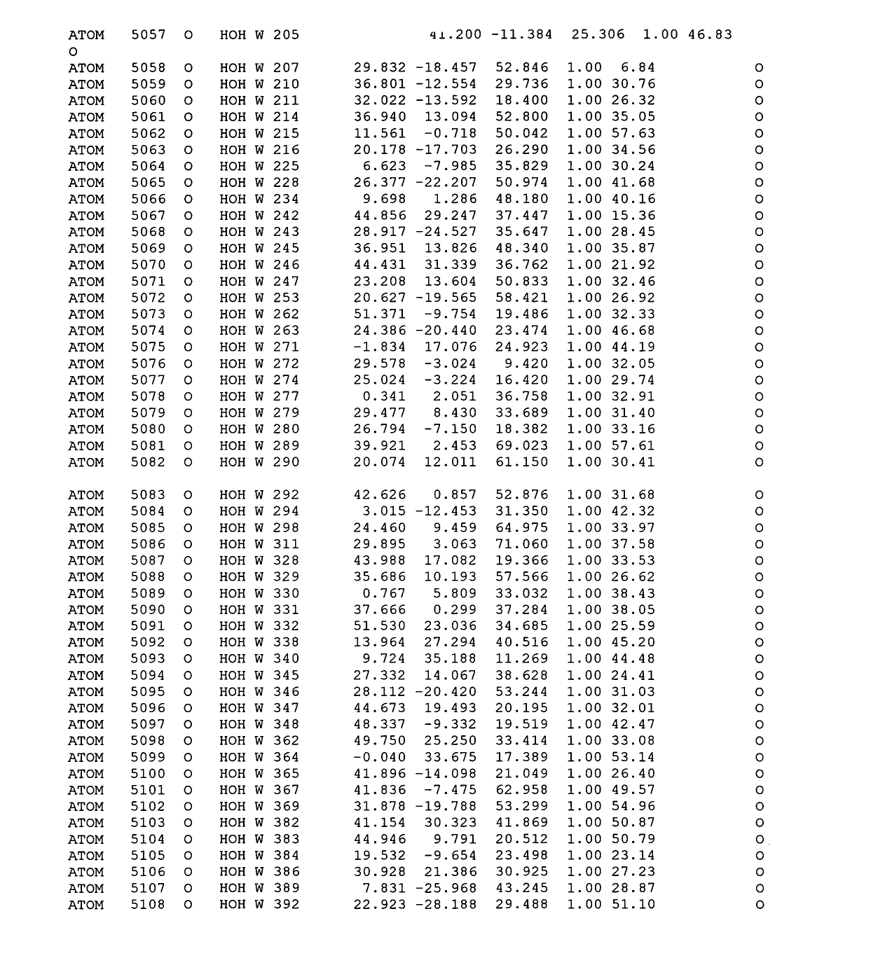

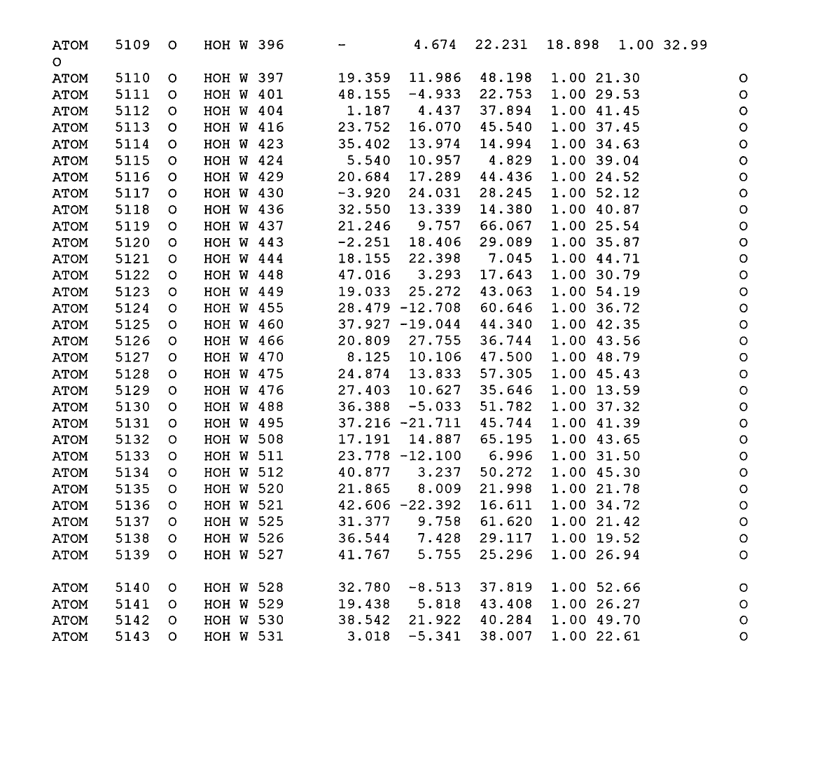

2. An isolated protein having the structure defined by the structural coordinates as set forth in FIG. 5

3. A crystal of protein CPT-2, wherein the crystal has unit cell dimensions of
a=b=66 Å to 70 Å, c=302 Å to 312 Å; α=β=γ=90°,
and the crystal has a P4₃2₁2 symmetry.

4. A crystal of protein CPT-2, wherein the crystal has unit cell dimensions of
a=93 Å to 97 Å, b=94 Å to 98 Å, c=304 Å to 312 Å; α=β=γ=90°,
and the crystal has a C222₁ symmetry.

5. A co-crystal of protein CPT-2 and a ligand bound to the active site of CPT-2, wherein the crystal has unit cell dimensions of
a=83 Å to 87 Å, b=94 Å to 98 Å, c from 121 Å to 127 Å; α=β=γ=90°,
and the crystal has a P2₁2₁2₁ symmetry.

6. A method of identifying compounds that can bind to protein CPT-2, comprising the steps of:
Applying a 3-dimensional molecular modeling algorithm to the atomic coordinates of protein CPT-2 shown in FIG. 4 to determine the spatial coordinates of the binding pocket of protein CPT-2; and
electronically screening the stored spatial coordinates of a set of candidate compounds against the spatial coordinates of the protein CPT-2 binding pocket to identify compound that can bind to protein CPT-2.

7. A method of identifying compounds that can bind to protein CPT-2, comprising the steps of:
Applying a 3-dimensional molecular modeling algorithm to the atomic coordinates of protein CPT-2 shown in FIG. 5 to determine the spatial coordinates of the binding pocket of protein CPT-2; and
electronically screening the stored spatial coordinates of a set of candidate compounds against the spatial coordinates of the protein CPT-2 binding pocket to identify compound that can bind to protein CPT-2.

8. A process for co-crystallizing CPT-2 with a ligand that binds to the active site, the process comprising
providing a buffered, aqueous solution of 3.75 to 50 mg/ml of CPT-2,
adding a molar excess of the ligand to the aqueous solution of CPT-2, and
growing crystals by vapor diffusion or microbatch using a buffered reservoir solution of 0% to 30% (w/v) PEG, wherein the PEG has an average molecular weight of 200 Da to 20,000 Da.

9. The process of claim 8, wherein the buffer reservoir additionally comprises 0 M to 2 M tri-ammonium citrate pH 7, 0 M to 1 M L-proline, 0 M to 1 M trimethylamine-N-oxide, 0 M to 1 M ammonium sulfate, 0 M to 1 M lithium sulfate, 0 M to 1 M ammonium acetate, 0 M to 1 M sodium or magnesium formate, and 0 M to 1 M D/L-malic acid pH 7.

10. A crystal produced by the process of claim 8.
